(19) United States
(12) Patent Application Publication
(10) Pub. No.: US 2024/0239790 A1
(43) Pub. Date: Jul. 18, 2024

(54) 7-Morpholino-1,6-Naphthyridin-5-yl Derivatives and Pharmaceutical Compositions Thereof Useful as DNA-PK Inhibitor

(71) Applicants: adMare Therapeutics Society, Vancouver (CA); LifeArc, London (GB); Provincial Health Services Authority, Vancouver (CA)

(72) Inventors: Stephen Paul Arns, North Vancouver (CA); Tom Han Hsiao Hsieh, Vancouver (CA); Fahimeh S. Shidmoossavee, North Vancouver (CA); Jason Samuel Tan, Richmond (CA); Leanna Yee, Vancouver (CA); Jay John Paquette, Surrey (CA); James Brian Jaquith, Cobourg (CA); Simon Andrew Osborne, London (GB); Ela Smiljanic-Hurley, London (GB); Callum Hamby, London (GB); Elliott Smyth, London (GB); Martin Ambler, London (GB); Andrew I. Minchinton, Vancouver (CA); Alastair H. Kyle, Vancouver (CA); Jennifer H.E. Baker, Vancouver (CA)

(21) Appl. No.: 18/279,379

(22) PCT Filed: Mar. 10, 2022
(86) PCT No.: PCT/CA2022/050356

§ 371 Date: Aug. 29, 2023

Related U.S. Application Data
(60) Provisional application No. 63/159,325, filed on Mar. 10, 2021.

(51) Int. Cl. C07D 471/04; A61K 31/5377; A61K 31/5386; A61K 31/541; A61K 31/55; A61K 45/06; A61K 48/00; A61P 35/00; C07D 519/00; C12N 9/22; C12N 15/11; C12N 15/63

(52) U.S. Cl. CPC C07D 471/04; A61K 31/5377; A61K 31/5386; A61K 31/541; A61K 31/55; A61K 45/06; A61K 48/005; A61P 35/00; C07D 519/00; C12N 9/22; C12N 15/111; C12N 15/63; C12N 2310/20

(57) ABSTRACT

The present disclosure provides compounds and methods for inhibiting DNA-dependent protein kinase (DNA-PK). Aspects of the present disclosure also include methods of using the compounds to treat diseases, including, but not limited to, cancer. In certain embodiments, the compounds inhibit DNA-PK and thus sensitize cancers to therapies such as chemotherapy and radiotherapy. Certain compounds of the present disclosure are in the form of prodrugs that release the DNA-PK inhibitor in hypoxic tissue such as is known to occur in cancers. Aspects of the present disclosure also include methods of using the compounds for repairing a DNA break in a target genomic region or for modifying expression of one or more genes or proteins. Compounds provided are of formula: (II)

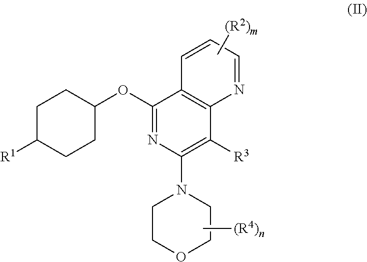

### CROSS-REFERENCE TO RELATED APPLICATIONS

This application claims the benefit to U.S. Provisional Application No. 63/159,325, filed Mar. 10, 2021, the disclosure of which is incorporated herein by reference.

### INTRODUCTION

Radiation therapy involves the exposure of a cancer to ionizing radiation (IR) at a dose that kill cells. Radiation therapy is administered as a beam of ionizing radiation or by implantation or temporary application of radioactive isotopes. Radiation therapy can be very effective, affording cure in a proportion of cases. Since it is not technically possible to selectively irradiate only the cancer cells, the dose-limiting factor associated with radiation therapy is the damage done to non-cancerous tissue. As a consequence, doses of radiation are prescribed which deliver the maximum dose of radiation to the tumor tissue, while exposing normal tissue to doses that produce tolerable side effects. IR causes a variety of cellular damage but it is the damage to the cell's DNA that is believed to be the primary cause of cell killing. The amount of DNA damage and the repair of that damage by DNA repair enzymes determines the extent of cell kill. Other forms of cancer therapy such as chemotherapy also cause DNA damage.

Cells have evolved pathways for the repair of genetic material caused either by endogenous metabolism or exogenous sources of ionizing radiation. The pathways that have evolved are often specific for the type of chemical lesions produced in DNA. IR produces a variety of lesions including base damage, single strand breaks, DNA-DNA and DNA-protein crosslinks and double strand breaks. However, the principle lethal event caused by IR used in radiotherapy is believed to be the induction of DNA double strand breaks (DSB). DSB's are repaired by several enzymatic pathways. One is non-homologous end-joining (NHEJ) that occurs in all phases of the cell cycle. DSB's can also be repaired by homologous recombination (HR) in cells where the repair machinery has access to a homologous strand of DNA from a sister chromatid. As a consequence, HR occurs primarily in late S and G2 phases of the cell cycle. Other mechanisms of end joining also occur.

Hypoxic cells (cells at lower than normal physiological oxygen tension) are commonly found in human tumors. They arise either because the cellular proliferation within tumors results in cells becoming located beyond the diffusion distance of oxygen from the nearest functioning blood vessel or as a result of temporary interruptions of blood flow. Hypoxic cells are resistant to ionizing radiation (IR) because molecular oxygen can react with the sites of initial molecule ionization making the damage more difficult to repair and because in the absence of oxygen spontaneous reductive reactions occur to restitute the original molecule. Thus, hypoxia reduces the effectiveness of radiotherapy. Clinical studies measuring oxygen tension in tumors and clinical trials of treatments which increase tumor oxygenation or drugs which act as oxygen mimetics have confirmed the role of hypoxic cells as an impediment to the effectiveness of radiation therapy. In addition, hypoxic tumors are also implicated as being a source of therapy resistance to chemotherapy.

DNA-PK (DNA-dependent protein kinase) is an enzyme involved in the repair of DNA DSBs. DNA-PK is a member of the PI3 kinase-like kinase (PIKK) family of atypical protein kinases. The important role of DNA-PK in cell survival following radiation therapy is well established. Small molecule DNA-PK inhibitors have demonstrated 2-fold or more radiosensitization of cells in vitro and have been shown to inhibit DSB repair. In addition, DNA-PK inhibition increases sensitivity to DNA damaging chemotherapy agents.

In addition, precise genome targeting technologies may be used to enable systematic engineering of genetic variations. The use of genome editing systems, such as Clustered Regularly Interspaced Short Palindromic Repeats (CRISPR)-endonuclease based genome editing technology has grown significantly over the past few years. The type II CRISPR-Cas9 bacterial innate immune system has been used as an effective genome editing tool for targeted modification of the human genome. Recently, CRISPR-Cpf genome editing systems have also been described. CRISPR-endonuclease based genome editing is dependent, in part, upon non-homologous end joining (NHEJ) and homology directed repair (HDR) pathways to repair DNA double strand breaks. DNA-PK inhibition has been demonstrated to increase the rate of HDR following Cas9-mediated DNA cleavage (Robert et al., Genome Medicine (2015) 7:93).

### SUMMARY

The present disclosure provides compounds and methods for inhibiting DNA-dependent protein kinase (DNA-PK). Aspects of the present disclosure also include methods of using the compounds to treat diseases, including, but not limited to, cancer. In certain embodiments, the compounds inhibit DNA-PK and thus sensitize cancers to therapies such as chemotherapy and radiotherapy. Certain compounds of the present disclosure are in the form of prodrugs that release the DNA-PK inhibitor in hypoxic tissue such as is known to occur in cancers. Aspects of the present disclosure also include methods of using the compounds for repairing a DNA break in a target genomic region or for modifying expression of one or more genes or proteins.

Aspects of the present disclosure include a compound of formula (II):

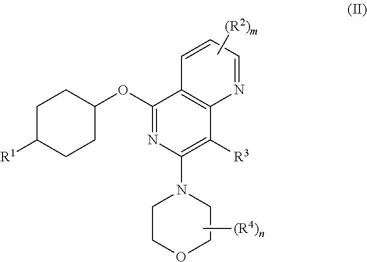

wherein:
R¹ is selected from 5- to 10-membered heteroaryl, NR⁶R⁷, C(O)R⁷, C(O)NR⁶R⁷, N═C(NR⁶R⁷)₂, wherein each heteroaryl is optionally substituted with from 1 to 5 R⁸ substituents; each R² is independently selected from C₂-C₆-alkynyl, halo, OR⁵, NR⁶R⁷, COOR⁶, C(O)NR⁶R⁷, C₃-C₈-cycloalkyl, 3- to 8-membered heterocycloalkyl, 5- to 10-membered aryl, 5- to 10-membered heteroaryl, S—C₁-C₆-alkyl, S(O)₂—C₁-C₆-alkyl, and S(O)₂—NR¹⁰R¹⁰, wherein each alkyl, alkynyl, cycloalkyl, heterocycloalkyl, aryl, heteroaryl is optionally substituted with from 1 to 5 R⁹ substituents;
R³ is selected from H, halo, C₁-C₆-alkyl and C₁-C₆-haloalkyl;
each R⁴ is independently selected from halo, C₁-C₆-alkyl and C₁-C₆-haloalkyl, wherein two R⁴ groups are optionally linked to form a 5- to 7-membered heterocycloalkyl;
each R⁵ is independently selected from H and C₁-C₆-alkyl, wherein each alkyl is optionally substituted with from 1 to 5 R⁹ substituents;
each R⁶ is independently selected from H, C₁-C₆-alkyl and COOR⁵;
each R⁷ is independently selected from H, OR⁵, cyano, C₁-C₆-alkyl, C₃-C₈-cycloalkyl, 3- to 10-membered heterocycloalkyl, 5- to 10-membered aryl, 5- to 10-membered heteroaryl, C(O)—C₁-C₆-alkyl, C(O)—(C₃-C₈-cycloalkyl), C(O)-(3- to 8-membered heterocycloalkyl), C(O)-(5- to 10-membered aryl), C(O)-(5- to 10-membered heteroaryl), C(O)—O—C₁-C₆-alkyl, S(O)₂—C₁-C₆-alkyl, S(O)₂—(C₃-C₈-cycloalkyl), S(O)₂-(3- to 8-membered heterocycloalkyl), S(O)₂-(5- to 10-membered aryl), S(O)₂-(5- to 10-membered heteroaryl), C(O)NR¹⁰R¹⁰, C(═NH)NH₂, and S(O)₂—NR¹⁰R¹⁰, wherein each alkyl, cycloalkyl, heterocycloalkyl, aryl and heteroaryl is optionally substituted with from 1 to 5 R¹¹ substituents;
each R⁸ is independently selected from C₁-C₆-alkyl, C₁-C₆-haloalkyl, and halo;
each R⁹ is independently selected from OH, oxo, C₁-C₆-alkyl, 3- to 8-membered heterocycloalkyl, and 5- to 10-membered aryl, wherein each alkyl, heterocycloalkyl and aryl is optionally substituted with from 1 to 5 R¹¹ substituents;
each R¹⁰ is independently selected from H, C₁-C₆-alkyl, C₁-C₆-alkoxy, 5- to 10-membered aryl, and S(O)₂—C₁-C₆-alkyl, wherein each alkyl and aryl is optionally substituted with from 1 to 5 R¹¹ substituents;
each R¹¹ is independently selected from OH, oxo, halo, cyano, C₁-C₆-alkyl, C₁-C₆-haloalkyl, C₁-C₆-alkoxy, C₃-C₈-cycloalkyl, 3- to 8-membered heterocycloalkyl, 5- to 10-membered aryl, 5- to 10-membered heteroaryl, NR⁶R⁷, C(O)NR⁶R⁷, C(O)OR⁷, and S(O)₂NR⁶R⁷, wherein each alkyl, haloalkyl, alkoxy, cycloalkyl, heterocycloalkyl, aryl and heteroaryl is optionally substituted with 1 to 5 R¹² substituents;
each R¹² is independently selected from halo, OH, oxo, cyano, C₁-C₆-alkyl, C₁-C₆-haloalkyl, C₁-C₆-alkoxy, 3- to 8-membered heterocycloalkyl, 5- to 10-membered heteroaryl, nitro, and NR¹⁰R¹⁰, wherein each alkyl, haloalkyl, alkoxy, heterocycloalkyl and heteroaryl is optionally substituted with 1 to 5 R¹³ substituents;
each R¹³ is independently selected from OH, halo, C₁-C₆-alkyl, and C₁-C₆-haloalkyl;
m is 0 or an integer selected from 1, 2 and 3; and
n is 0 or an integer selected from 1, 2, 3 and 4,
or a prodrug or a pharmaceutically acceptable salt thereof.

In some embodiments, m is 0.

In some embodiments, n is 0.

In some embodiments, R¹ is C(O)NR⁶R⁷. In some embodiments, R¹ is N═C(NR⁶R⁷)₂.

In some embodiments, R² is C₂-C₆-alkynyl. In some embodiments, R² is COOR⁶.

In some embodiments, R² is C(O)NR⁶R⁷. In some embodiments, R² is 5- to 10-membered aryl or 5- to 10-membered heteroaryl. In some embodiments, R² is 3- to 8-membered heterocycloalkyl. In some embodiments, R² is S—C₁-C₆-alkyl or S(O)₂—C₁-C₆-alkyl.

In some embodiments, R³ is H.

In some embodiments, R⁴ is halo.

In some embodiments, n is 2, and two R⁴ groups are linked to form a 5- to 7-membered heterocycloalkyl.

In some embodiments, the compound is of formula (IIa):

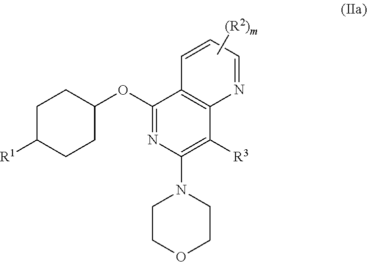

wherein:
R¹ is NR⁶R⁷;
R⁶ is H;
R⁷ is selected from C(O)—C₁-C₆-alkyl, C(O)—(C₃-C₈-cycloalkyl), C(O)-(3- to 8-membered heterocycloalkyl), C(O)-(5- to 10-membered aryl), C(O)-(5- to 10-membered heteroaryl), C(O)NR¹⁰R¹⁰, and C(═NH)NH₂, wherein each alkyl, cycloalkyl, heterocycloalkyl, aryl and heteroaryl is optionally substituted with from 1 to 5 R¹¹ substituents.

In some embodiments, the compound is of formula (IIb):

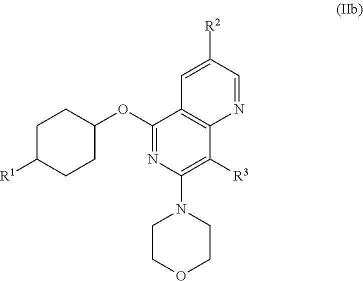

wherein:
R² is NR⁶R⁷;
R⁶ is H;
R⁷ is selected from C(O)NR¹⁰R¹⁰, S(O)₂—C₁-C₆-alkyl, S(O)₂—(C₃-C₈-cycloalkyl), S(O)₂-(3- to 8-membered heterocycloalkyl), S(O)₂-(5- to 10-membered aryl), S(O)₂-(5- to 10-membered heteroaryl), and S(O)₂—NR¹⁰R¹⁰, wherein each alkyl, cycloalkyl, heterocycloalkyl, aryl and heteroaryl is optionally substituted with from 1 to 5 R¹¹ substituents.

In some embodiments, the compound is of formula (IIc):

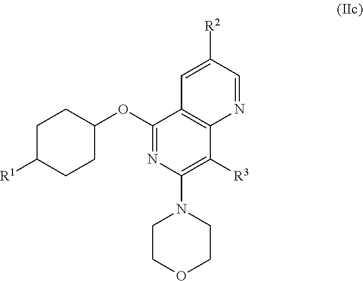

wherein:
R² is OR⁵;
R⁵ is C₁-C₆-alkyl substituted with from 1 to 5 R⁹ substituents.

In some embodiments, the compound is of formula (IId):

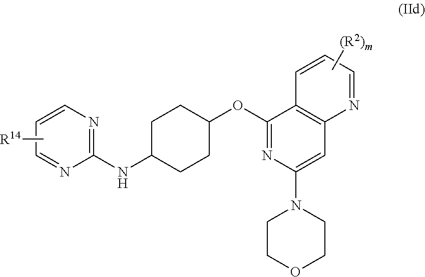

wherein:
R¹⁴ is C₁-C₆-alkoxy, wherein the alkoxy is substituted with 1 to 2 R¹⁵ substituents,
wherein when the alkoxy of R¹⁴ is substituted with one R¹⁵ substituent, R¹⁵ is selected from 3- to 8-membered heterocycloalkyl, 5- to 10-membered heteroaryl, and NR¹⁷R¹⁷, wherein each heterocycloalkyl and heteroaryl is optionally substituted with 1 to 2 R¹⁶ substituents;
wherein when the alkoxy of R¹⁴ is substituted with two R¹⁵ substituents, each R¹⁵ is independently selected from OH, oxo, 3- to 8-membered heterocycloalkyl, and NR¹⁷R¹⁷, wherein heterocycloalkyl is optionally substituted with 1 to 2 R¹⁶ substituents;
each R¹⁶ is independently selected from OH, halo, and C₁-C₆-alkyl;
each R¹⁷ is independently selected from H, C₁-C₆-alkyl, C₁-C₆-alkoxy, 5- to 10-membered aryl, and S(O)₂—C₁-C₆-alkyl, wherein each alkyl and aryl is optionally substituted with from 1 to 2 R¹⁸ substituents;
each R¹⁸ is independently selected from OH, oxo, halo, cyano, C₁-C₆-alkyl, C₁-C₆-haloalkyl, C₁-C₆-alkoxy, C₃-C₈-cycloalkyl, 3- to 8-membered heterocycloalkyl, 5- to 10-membered aryl, 5- to 10-membered heteroaryl, NR¹⁹R²⁰, C(O)NR¹⁹R²⁰, C(O)OR²⁰, and S(O)₂NR¹⁹R²⁰; and
each of R¹⁹ and R²⁰ is independently selected from H and C₁-C₆-alkyl.

In some embodiments, the compound of formula (II) has the following features:
R¹ is NR⁶R⁷;
R⁶ is H;
R⁷ is 5- to 10-membered heteroaryl, wherein heteroaryl is substituted with C₁-C₆-alkyl, wherein alkyl is substituted with oxo and NR²¹R²², wherein each or R²¹ and R²² is independently selected from H and C₁-C₆-alkyl.

In some embodiments, the compound of formula (II) has the following features:
R¹ is NR⁶R⁷;
R⁶ is H;
R⁷ is 5- to 10-membered heteroaryl, wherein heteroaryl is substituted with from 2 to 3 R¹¹ substituents;
wherein one R¹¹ substituent is oxo, and each additional R¹¹ substituent is independently selected from OH, halo, cyano, C₁-C₆-alkyl, C₁-C₆-haloalkyl, C₁-C₆-alkoxy, C₃-C₈-cycloalkyl, 3- to 8-membered heterocycloalkyl, 5- to 10-membered aryl, 5- to 10-membered heteroaryl, NR²³R²⁴, C(O)NR²³R²⁴, C(O)OR²⁴, and S(O)₂NR²³R²⁴, wherein each alkyl, haloalkyl, alkoxy, cycloalkyl, heterocycloalkyl, aryl and heteroaryl is optionally substituted with 1 to 5 R¹² substituents;
each of R²³ and R²⁴ is independently selected from H and C₁-C₆-alkyl.

In some embodiments, the compound is selected from:

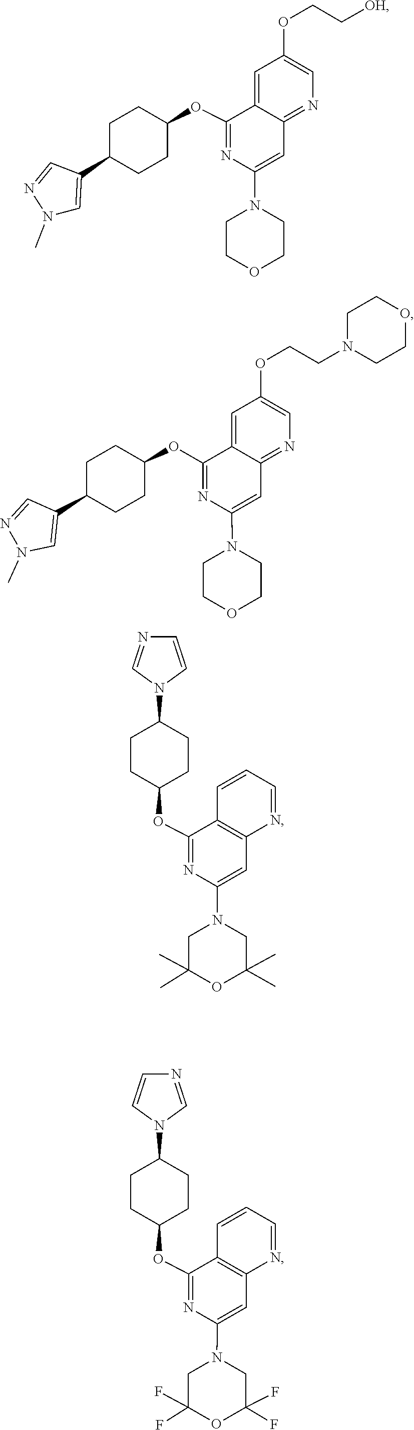 [SMILES: Cn1cc([C@H]2CC[C@@H](Oc3nc(N4CCOCC4)cc4ncc(OCCO)cc34)CC2)cn1 ; Cn1cc([C@H]2CC[C@@H](Oc3nc(N4CCOCC4)cc4ncc(OCCN5CCOCC5)cc34)CC2)cn1 ; CC1(C)CN(c2cc3ncccc3c(O[C@H]3CC[C@@H](n4ccnc4)CC3)n2)CC(C)(C)O1 ; FC1(F)CN(c2cc3ncccc3c(O[C@H]3CC[C@@H](n4ccnc4)CC3)n2)CC(F)(F)O1]

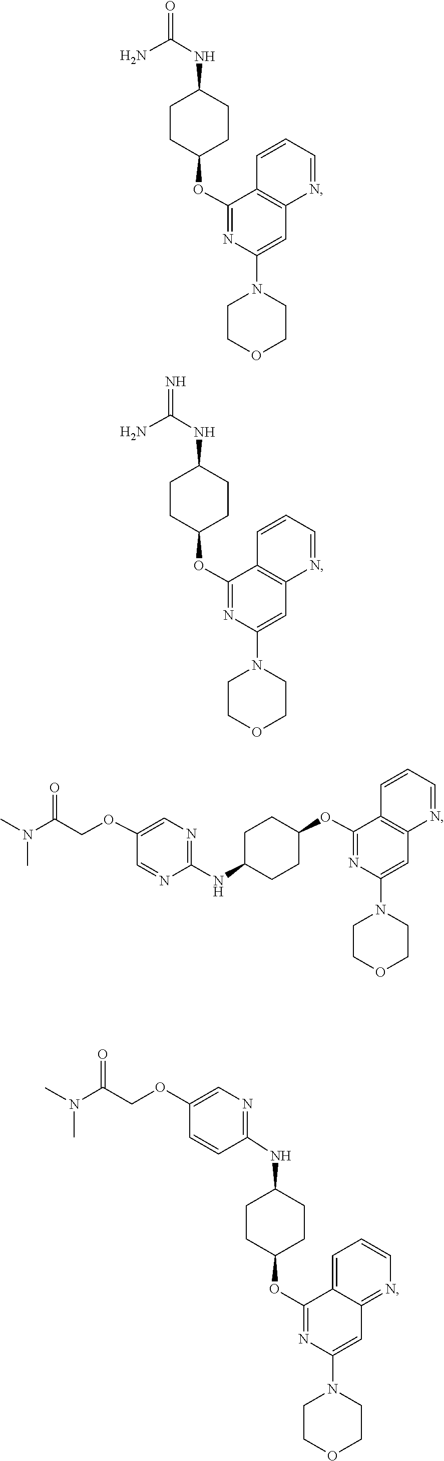 [SMILES: NC(=O)N[C@H]1CC[C@@H](Oc2nc(N3CCOCC3)cc3ncccc23)CC1 ; N=C(N)N[C@H]1CC[C@@H](Oc2nc(N3CCOCC3)cc3ncccc23)CC1 ; CN(C)C(=O)COc1cnc(N[C@H]2CC[C@@H](Oc3nc(N4CCOCC4)cc4ncccc34)CC2)nc1 ; CN(C)C(=O)COc1ccc(N[C@H]2CC[C@@H](Oc3nc(N4CCOCC4)cc4ncccc34)CC2)nc1]

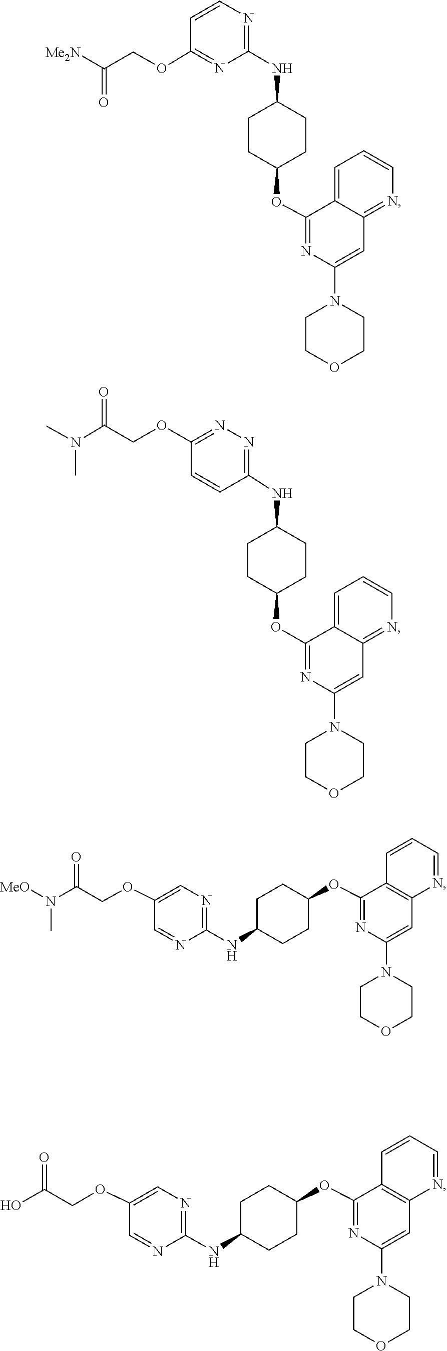 [SMILES: CN(C)C(=O)COc1ccnc(N[C@H]2CC[C@@H](Oc3nc(N4CCOCC4)cc4ncccc34)CC2)n1 ; CN(C)C(=O)COc1ccc(N[C@H]2CC[C@@H](Oc3nc(N4CCOCC4)cc4ncccc34)CC2)nn1 ; CON(C)C(=O)COc1cnc(N[C@H]2CC[C@@H](Oc3nc(N4CCOCC4)cc4ncccc34)CC2)nc1 ; O=C(O)COc1cnc(N[C@H]2CC[C@@H](Oc3nc(N4CCOCC4)cc4ncccc34)CC2)nc1]

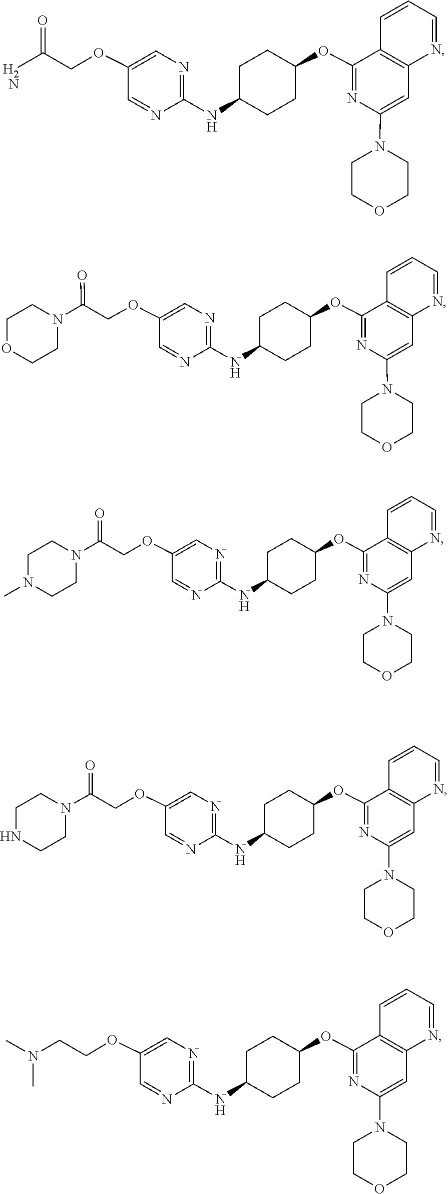 [SMILES: NC(=O)COc1cnc(N[C@H]2CC[C@@H](Oc3nc(N4CCOCC4)cc4ncccc34)CC2)nc1 ; O=C(COc1cnc(N[C@H]2CC[C@@H](Oc3nc(N4CCOCC4)cc4ncccc34)CC2)nc1)N1CCOCC1 ; CN1CCN(C(=O)COc2cnc(N[C@H]3CC[C@@H](Oc4nc(N5CCOCC5)cc5ncccc45)CC3)nc2)CC1 ; O=C(COc1cnc(N[C@H]2CC[C@@H](Oc3nc(N4CCOCC4)cc4ncccc34)CC2)nc1)N1CCNCC1 ; CN(C)CCOc1cnc(N[C@H]2CC[C@@H](Oc3nc(N4CCOCC4)cc4ncccc34)CC2)nc1]

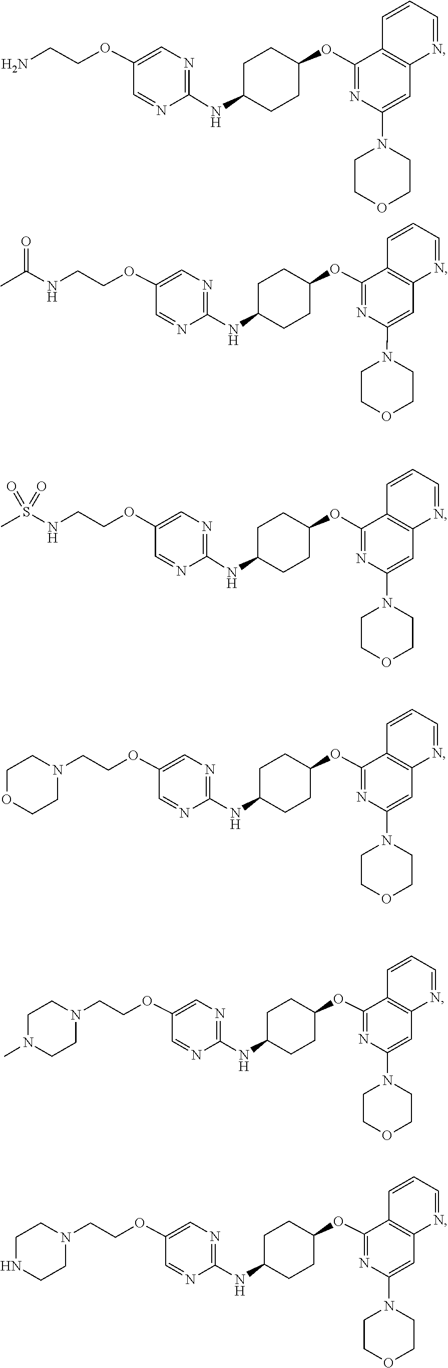

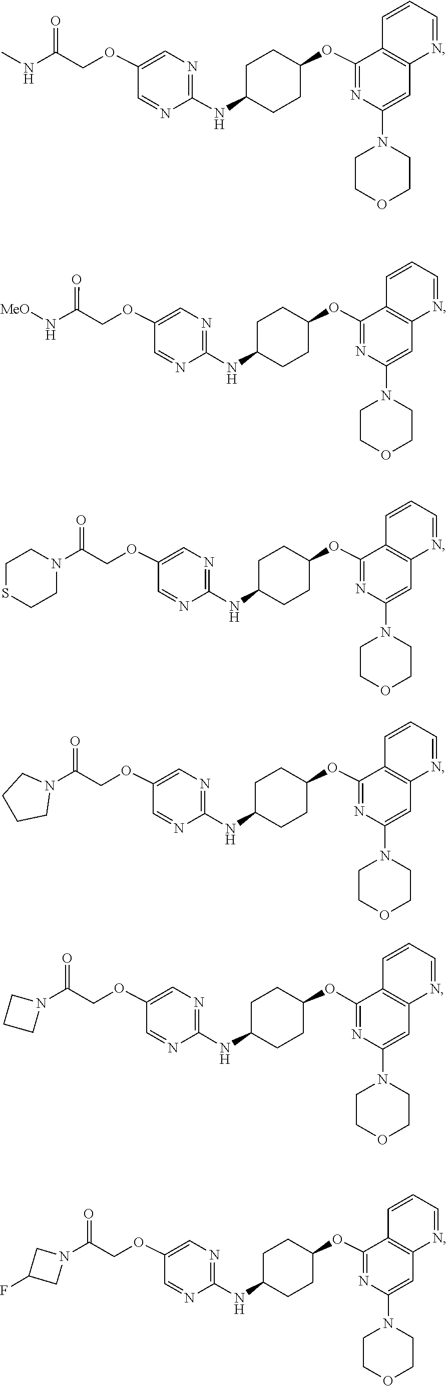

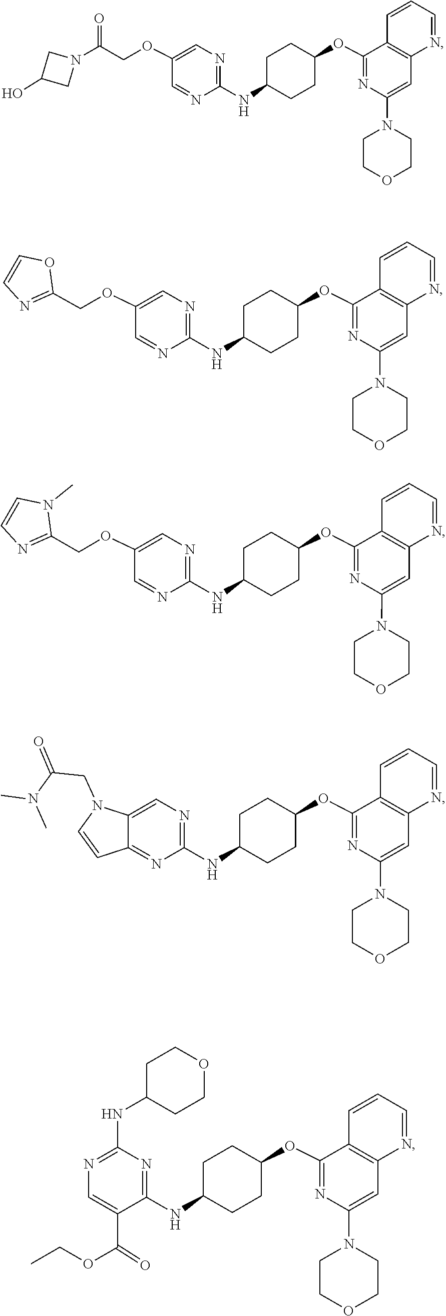 [SMILES: O=C(COc1cnc(N[C@H]2CC[C@@H](Oc3nc(N4CCOCC4)cc4ncccc34)CC2)nc1)N1CC(O)C1 ; c1cnc2cc(N3CCOCC3)nc(O[C@H]3CC[C@@H](Nc4ncc(OCc5ncco5)cn4)CC3)c2c1 ; Cn1ccnc1COc1cnc(N[C@H]2CC[C@@H](Oc3nc(N4CCOCC4)cc4ncccc34)CC2)nc1 ; CN(C)C(=O)Cn1ccc2nc(N[C@H]3CC[C@@H](Oc4nc(N5CCOCC5)cc5ncccc45)CC3)ncc21 ; CCOC(=O)c1cnc(NC2CCOCC2)nc1N[C@H]1CC[C@@H](Oc2nc(N3CCOCC3)cc3ncccc23)CC1]

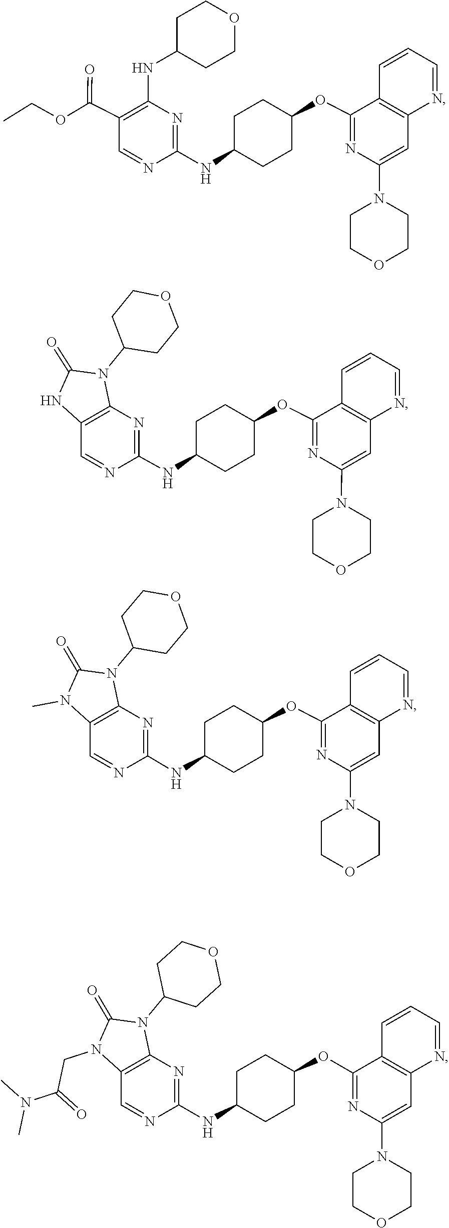 [SMILES: CCOC(=O)c1cnc(N[C@H]2CC[C@@H](Oc3nc(N4CCOCC4)cc4ncccc34)CC2)nc1NC1CCOCC1 ; O=c1[nH]c2cnc(N[C@H]3CC[C@@H](Oc4nc(N5CCOCC5)cc5ncccc45)CC3)nc2n1C1CCOCC1 ; Cn1c(=O)n(C2CCOCC2)c2nc(N[C@H]3CC[C@@H](Oc4nc(N5CCOCC5)cc5ncccc45)CC3)ncc21 ; CN(C)C(=O)Cn1c(=O)n(C2CCOCC2)c2nc(N[C@H]3CC[C@@H](Oc4nc(N5CCOCC5)cc5ncccc45)CC3)ncc21]

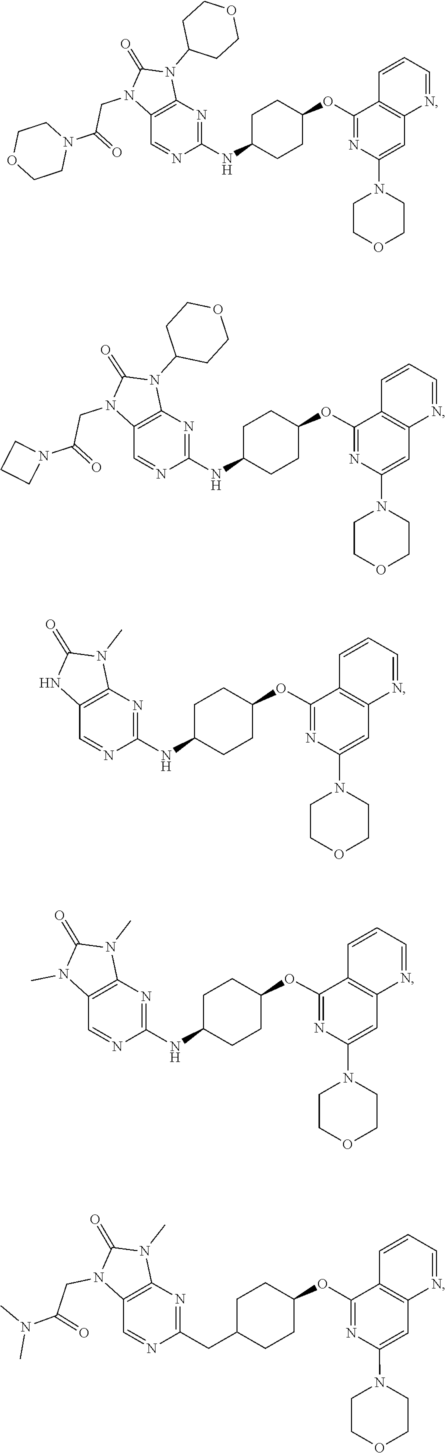

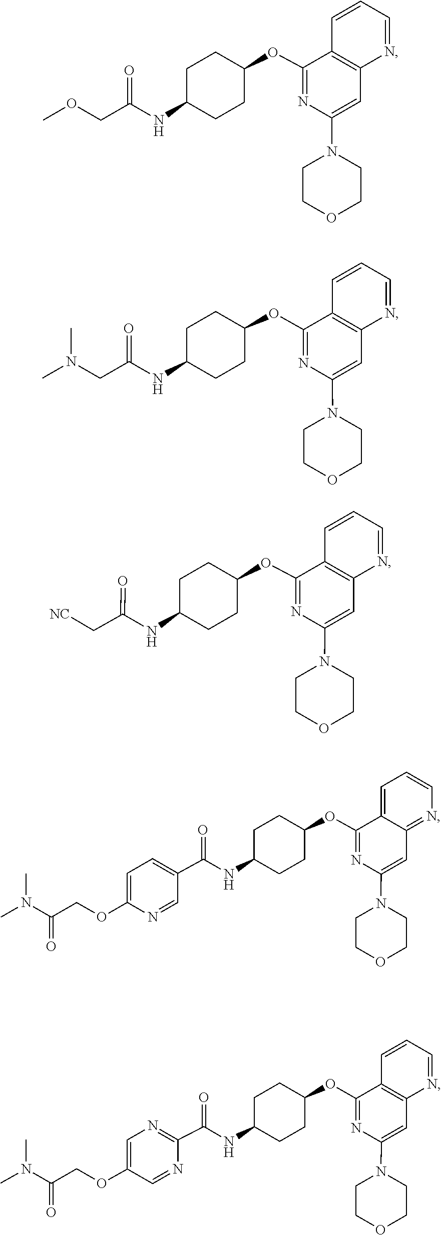

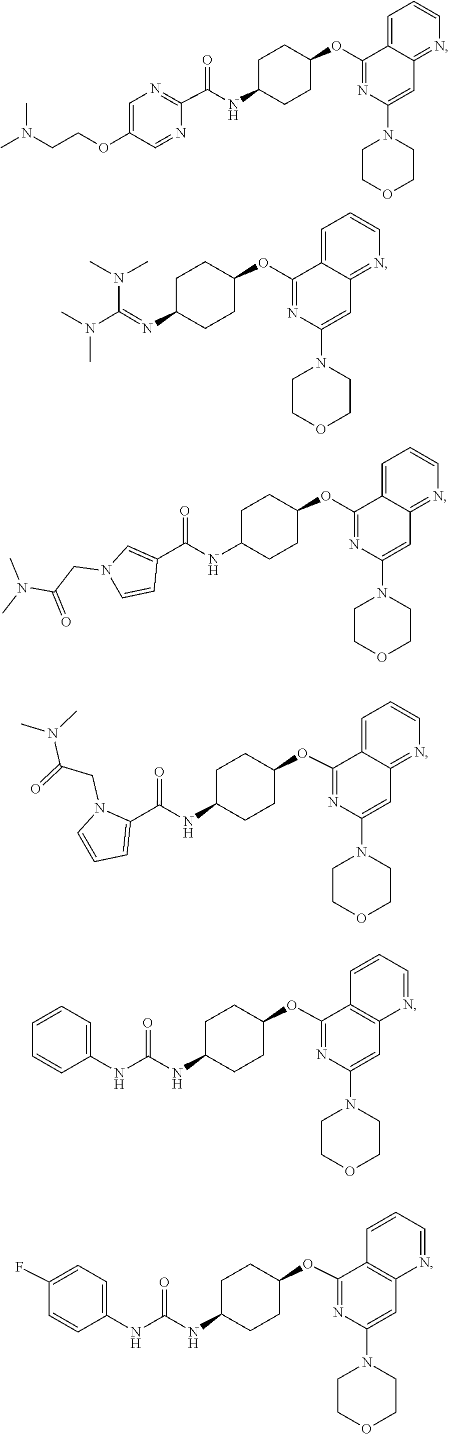

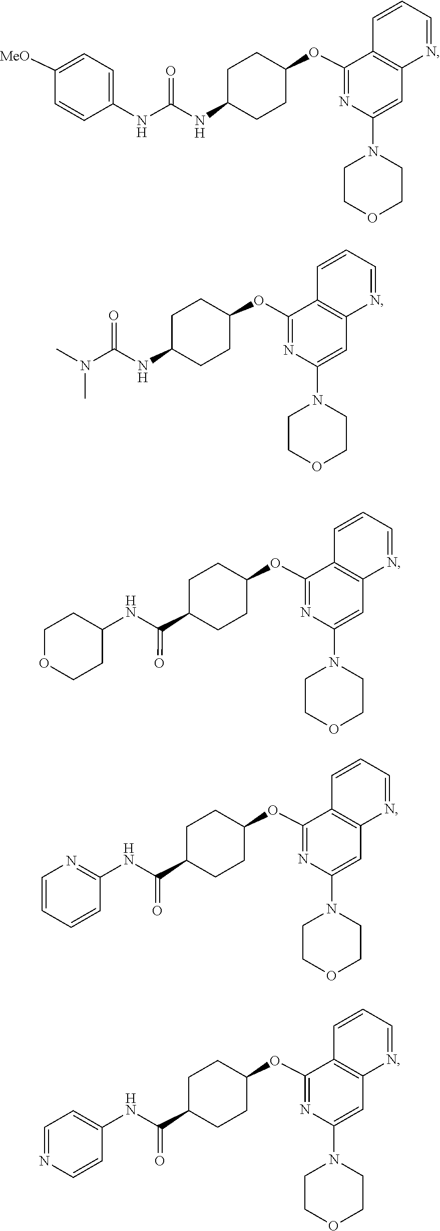

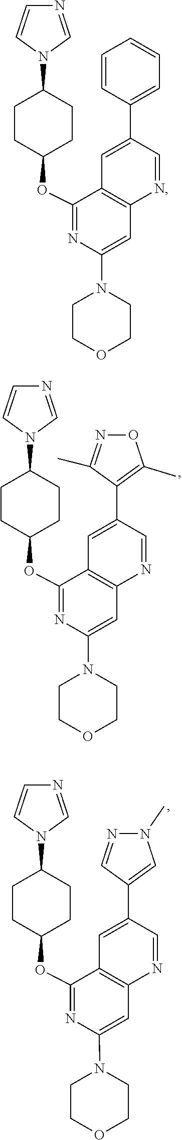 [SMILES: c1ccc(-c2cnc3cc(N4CCOCC4)nc(O[C@H]4CC[C@@H](n5ccnc5)CC4)c3c2)cc1 ; Cc1noc(C)c1-c1cnc2cc(N3CCOCC3)nc(O[C@H]3CC[C@@H](n4ccnc4)CC3)c2c1 ; Cn1cc(-c2cnc3cc(N4CCOCC4)nc(O[C@H]4CC[C@@H](n5ccnc5)CC4)c3c2)cn1]

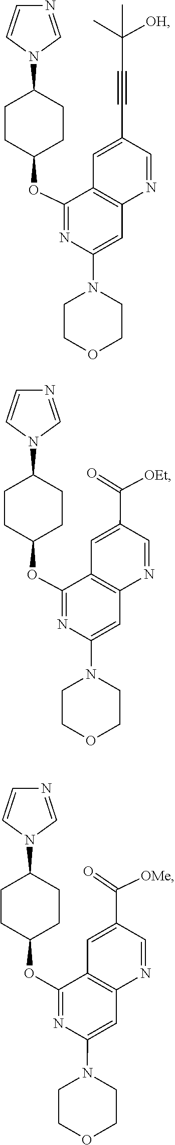 [SMILES: CC(C)(O)C#Cc1cnc2cc(N3CCOCC3)nc(O[C@H]3CC[C@@H](n4ccnc4)CC3)c2c1 ; CCOC(=O)c1cnc2cc(N3CCOCC3)nc(O[C@H]3CC[C@@H](n4ccnc4)CC3)c2c1 ; COC(=O)c1cnc2cc(N3CCOCC3)nc(O[C@H]3CC[C@@H](n4ccnc4)CC3)c2c1]

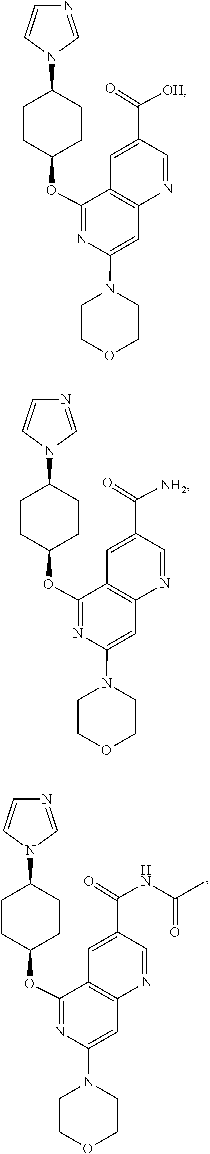 [SMILES: O=C(O)c1cnc2cc(N3CCOCC3)nc(O[C@H]3CC[C@@H](n4ccnc4)CC3)c2c1 ; NC(=O)c1cnc2cc(N3CCOCC3)nc(O[C@H]3CC[C@@H](n4ccnc4)CC3)c2c1 ; CC(=O)NC(=O)c1cnc2cc(N3CCOCC3)nc(O[C@H]3CC[C@@H](n4ccnc4)CC3)c2c1]

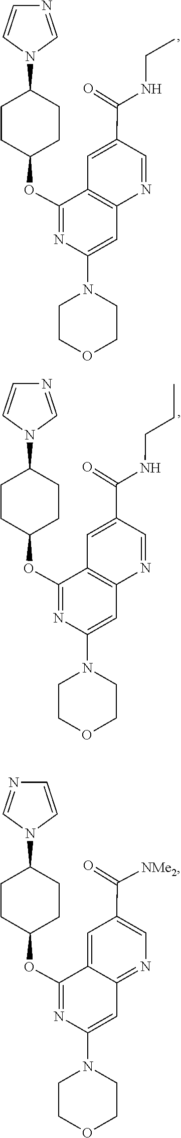

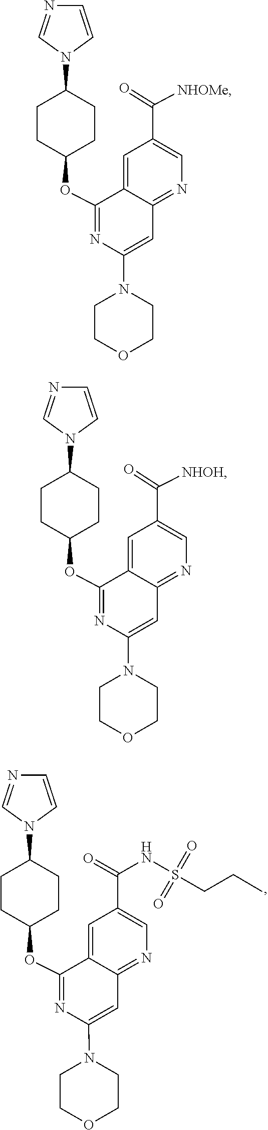

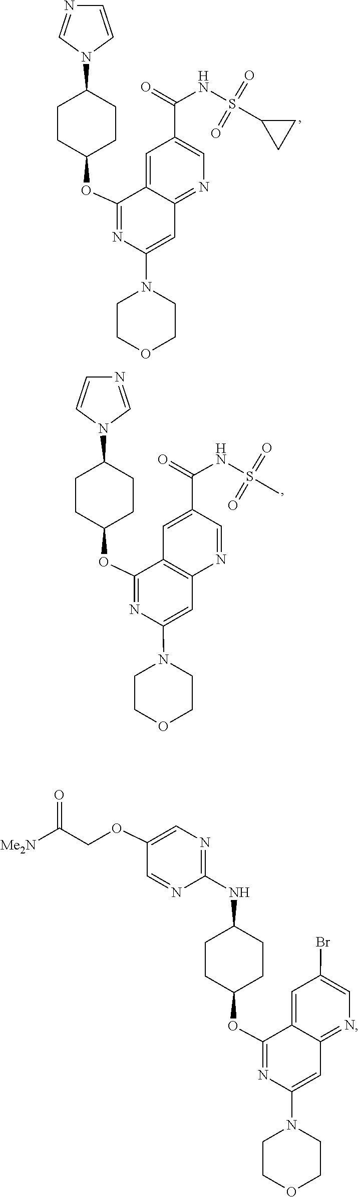

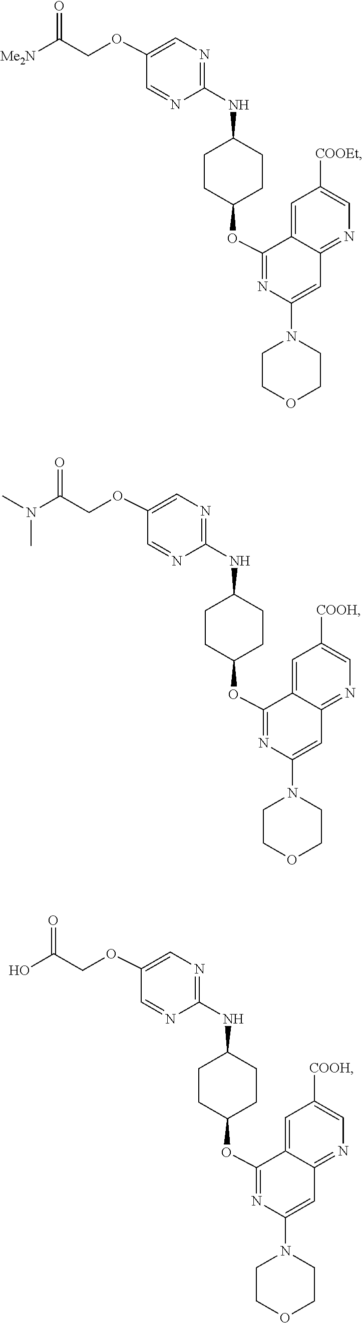 [SMILES: CCOC(=O)c1cnc2cc(N3CCOCC3)nc(O[C@H]3CC[C@@H](Nc4ncc(OCC(=O)N(C)C)cn4)CC3)c2c1 ; CN(C)C(=O)COc1cnc(N[C@H]2CC[C@@H](Oc3nc(N4CCOCC4)cc4ncc(C(=O)O)cc34)CC2)nc1 ; O=C(O)COc1cnc(N[C@H]2CC[C@@H](Oc3nc(N4CCOCC4)cc4ncc(C(=O)O)cc34)CC2)nc1]

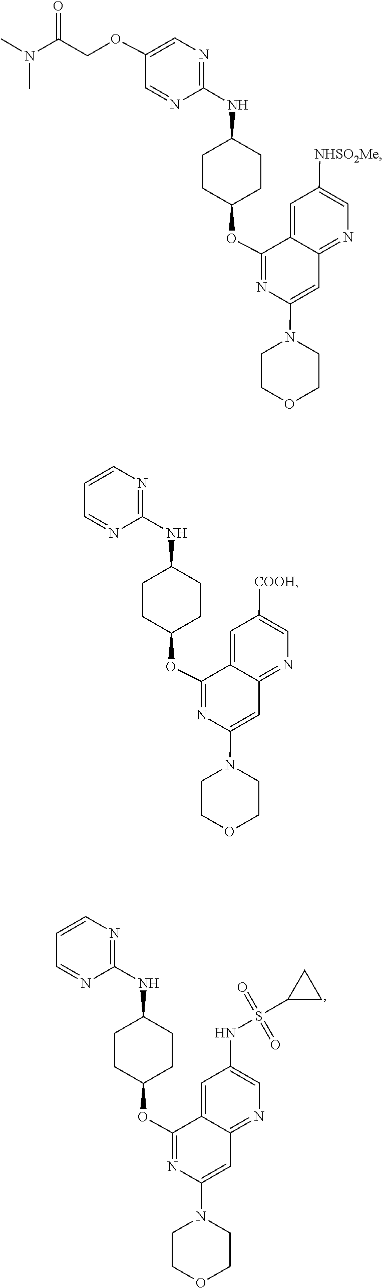

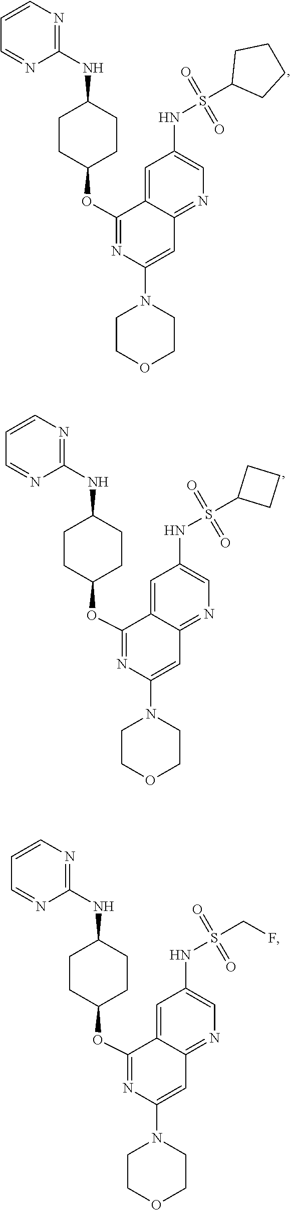 [SMILES: O=S(=O)(Nc1cnc2cc(N3CCOCC3)nc(O[C@H]3CC[C@@H](Nc4ncccn4)CC3)c2c1)C1CCCC1 ; O=S(=O)(Nc1cnc2cc(N3CCOCC3)nc(O[C@H]3CC[C@@H](Nc4ncccn4)CC3)c2c1)C1CCC1 ; O=S(=O)(CF)Nc1cnc2cc(N3CCOCC3)nc(O[C@H]3CC[C@@H](Nc4ncccn4)CC3)c2c1]

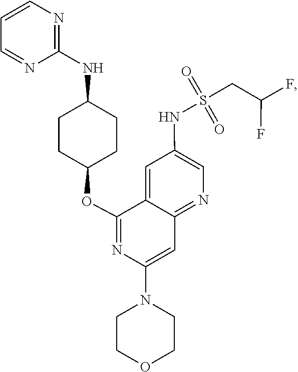 [SMILES: O=S(=O)(CC(F)F)Nc1cnc2cc(N3CCOCC3)nc(O[C@H]3CC[C@@H](Nc4ncccn4)CC3)c2c1]

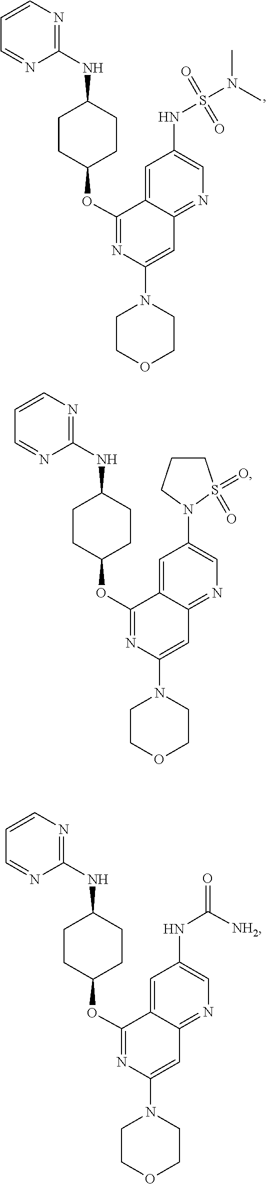 [SMILES: CN(C)S(=O)(=O)Nc1cnc2cc(N3CCOCC3)nc(O[C@H]3CC[C@@H](Nc4ncccn4)CC3)c2c1 ; O=S1(=O)CCCN1c1cnc2cc(N3CCOCC3)nc(O[C@H]3CC[C@@H](Nc4ncccn4)CC3)c2c1 ; NC(=O)Nc1cnc2cc(N3CCOCC3)nc(O[C@H]3CC[C@@H](Nc4ncccn4)CC3)c2c1]

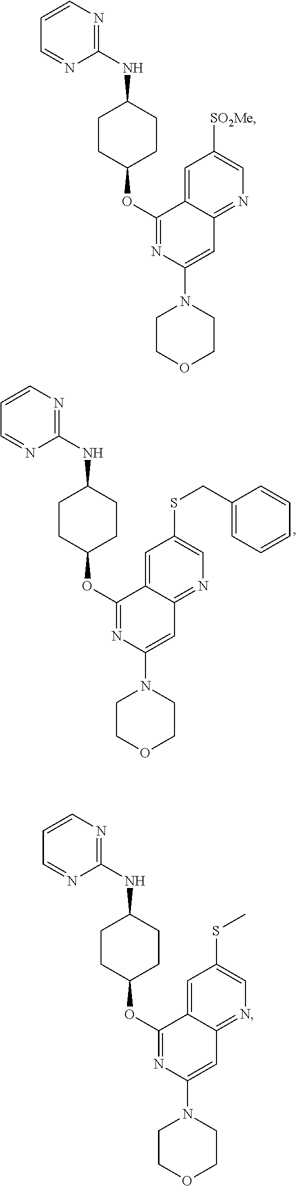 [SMILES: CS(=O)(=O)c1cnc2cc(N3CCOCC3)nc(O[C@H]3CC[C@@H](Nc4ncccn4)CC3)c2c1 ; c1ccc(CSc2cnc3cc(N4CCOCC4)nc(O[C@H]4CC[C@@H](Nc5ncccn5)CC4)c3c2)cc1 ; CSc1cnc2cc(N3CCOCC3)nc(O[C@H]3CC[C@@H](Nc4ncccn4)CC3)c2c1]

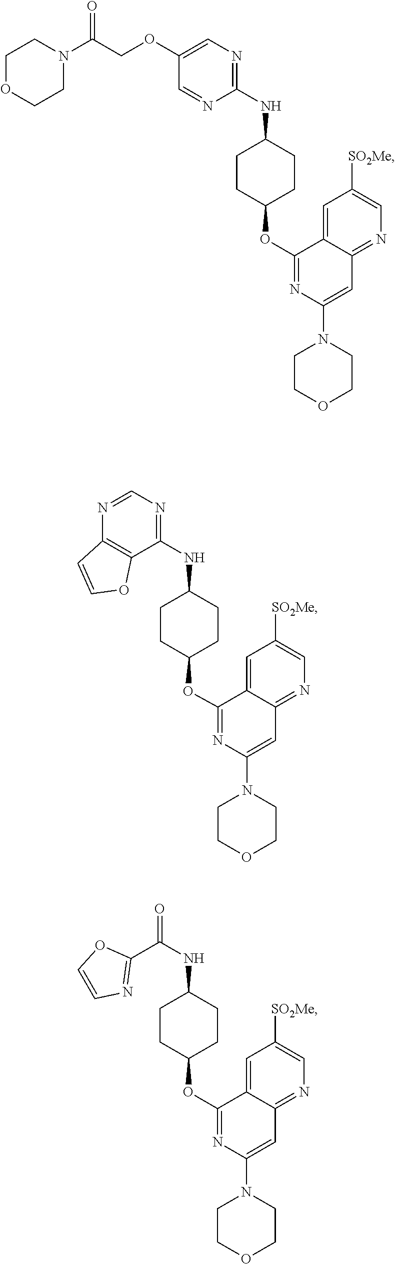 [SMILES: CS(=O)(=O)c1cnc2cc(N3CCOCC3)nc(O[C@H]3CC[C@@H](Nc4ncc(OCC(=O)N5CCOCC5)cn4)CC3)c2c1 ; CS(=O)(=O)c1cnc2cc(N3CCOCC3)nc(O[C@H]3CC[C@@H](Nc4ncnc5ccoc45)CC3)c2c1 ; CS(=O)(=O)c1cnc2cc(N3CCOCC3)nc(O[C@H]3CC[C@@H](NC(=O)c4ncco4)CC3)c2c1]

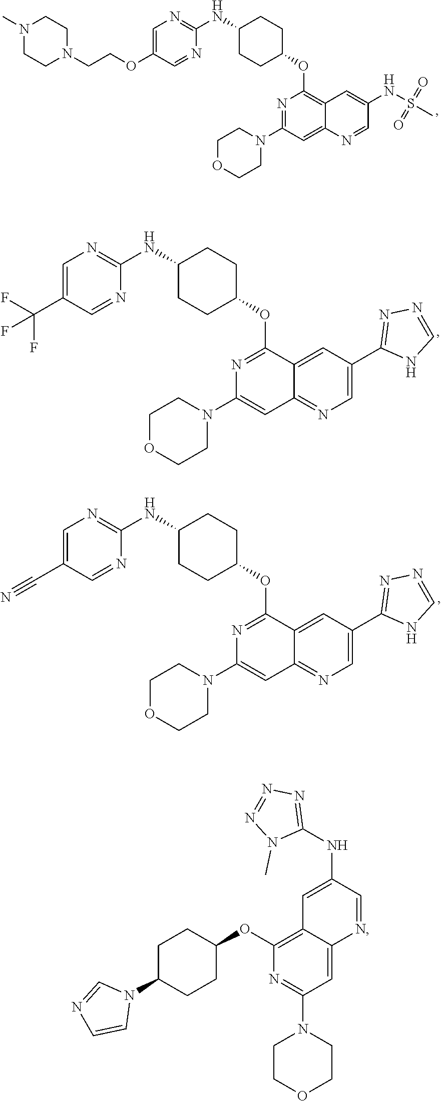

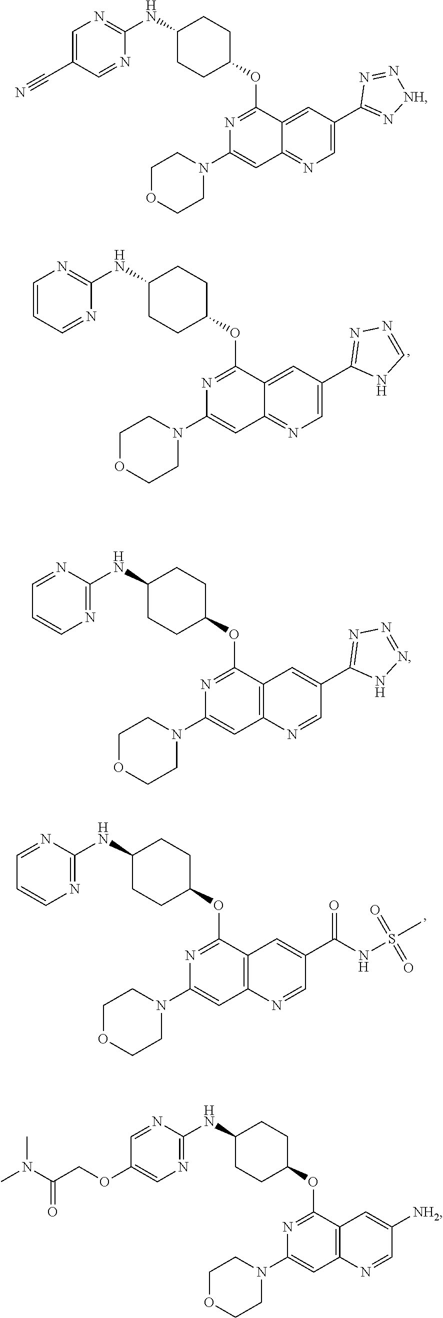

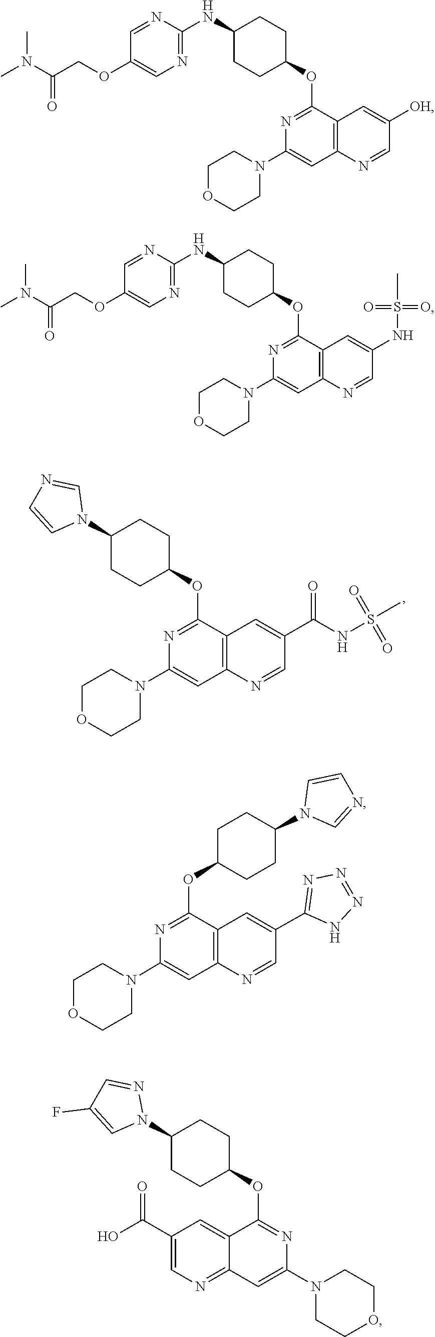

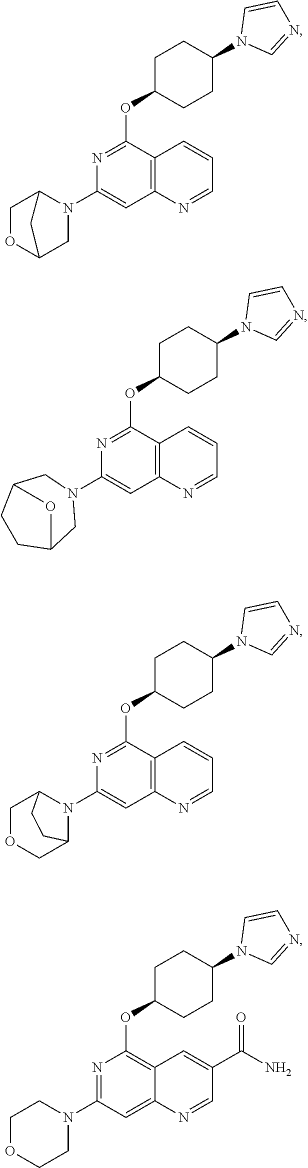 [SMILES: c1cnc2cc(N3CC4CC3CO4)nc(O[C@H]3CC[C@@H](n4ccnc4)CC3)c2c1 ; c1cnc2cc(N3CC4CCC(C3)O4)nc(O[C@H]3CC[C@@H](n4ccnc4)CC3)c2c1 ; c1cnc2cc(N3C4CCC3COC4)nc(O[C@H]3CC[C@@H](n4ccnc4)CC3)c2c1 ; NC(=O)c1cnc2cc(N3CCOCC3)nc(O[C@H]3CC[C@@H](n4ccnc4)CC3)c2c1]

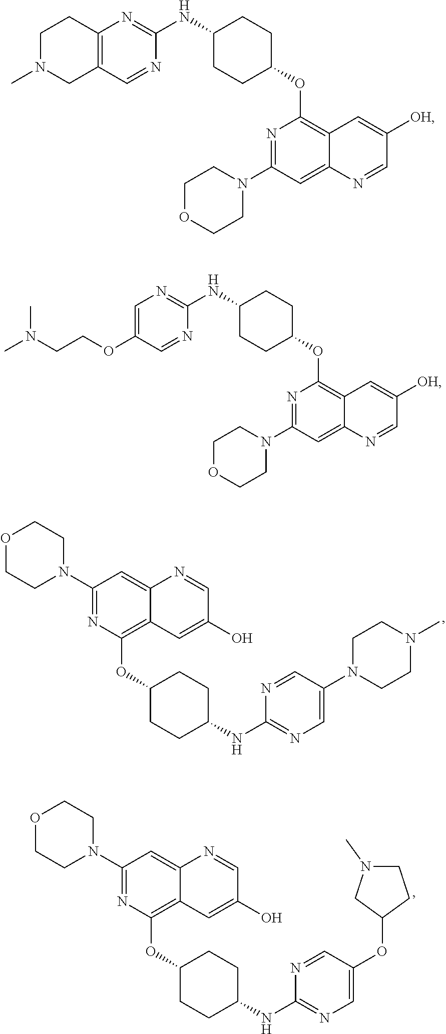 [SMILES: CN1CCc2nc(N[C@H]3CC[C@@H](Oc4nc(N5CCOCC5)cc5ncc(O)cc45)CC3)ncc2C1 ; CN(C)CCOc1cnc(N[C@H]2CC[C@@H](Oc3nc(N4CCOCC4)cc4ncc(O)cc34)CC2)nc1 ; CN1CCN(c2cnc(N[C@H]3CC[C@@H](Oc4nc(N5CCOCC5)cc5ncc(O)cc45)CC3)nc2)CC1 ; CN1CCC(Oc2cnc(N[C@H]3CC[C@@H](Oc4nc(N5CCOCC5)cc5ncc(O)cc45)CC3)nc2)C1]

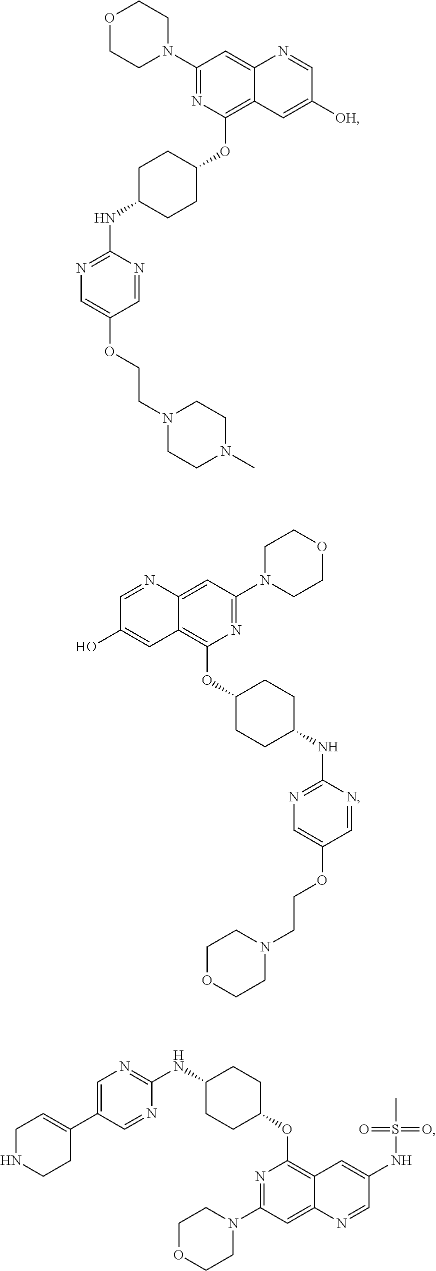 [SMILES: CN1CCN(CCOc2cnc(N[C@H]3CC[C@@H](Oc4nc(N5CCOCC5)cc5ncc(O)cc45)CC3)nc2)CC1 ; Oc1cnc2cc(N3CCOCC3)nc(O[C@H]3CC[C@@H](Nc4ncc(OCCN5CCOCC5)cn4)CC3)c2c1 ; CS(=O)(=O)Nc1cnc2cc(N3CCOCC3)nc(O[C@H]3CC[C@@H](Nc4ncc(C5=CCNCC5)cn4)CC3)c2c1]

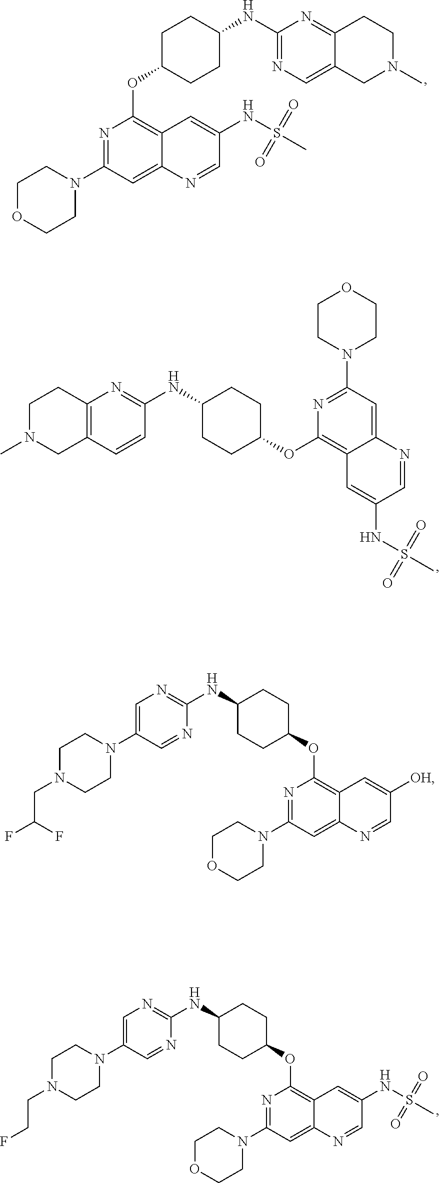

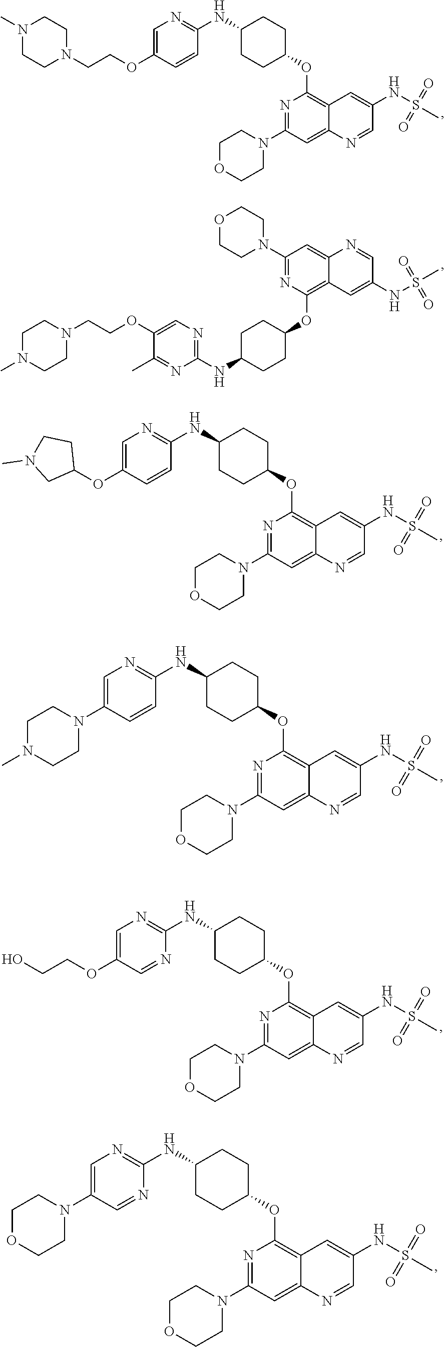

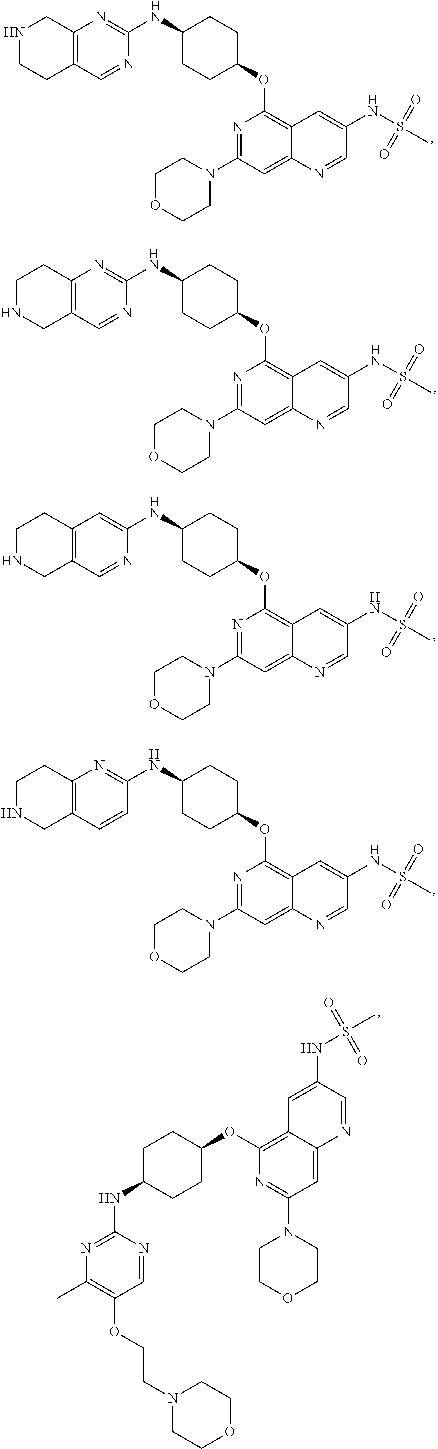

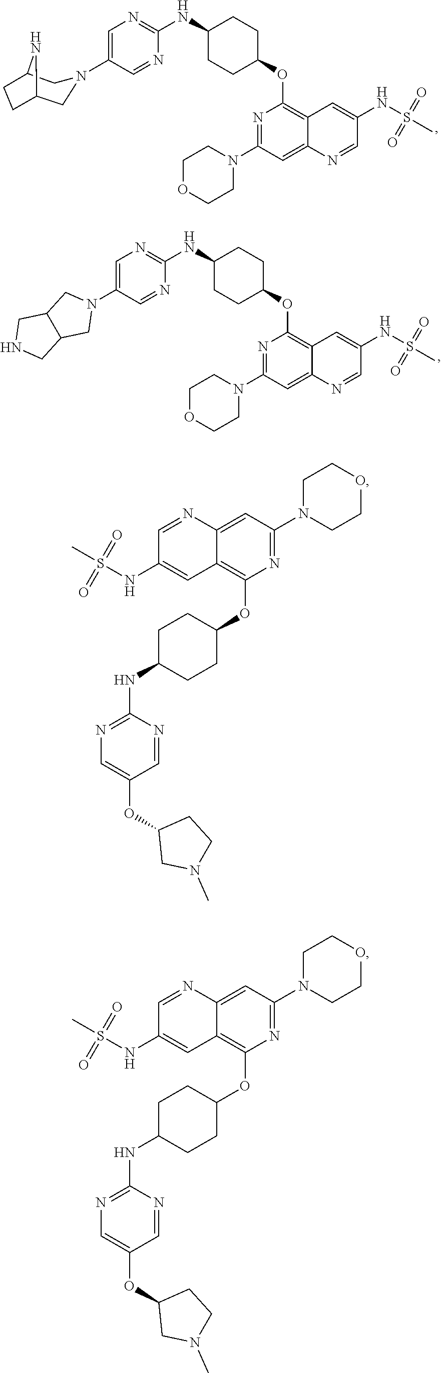

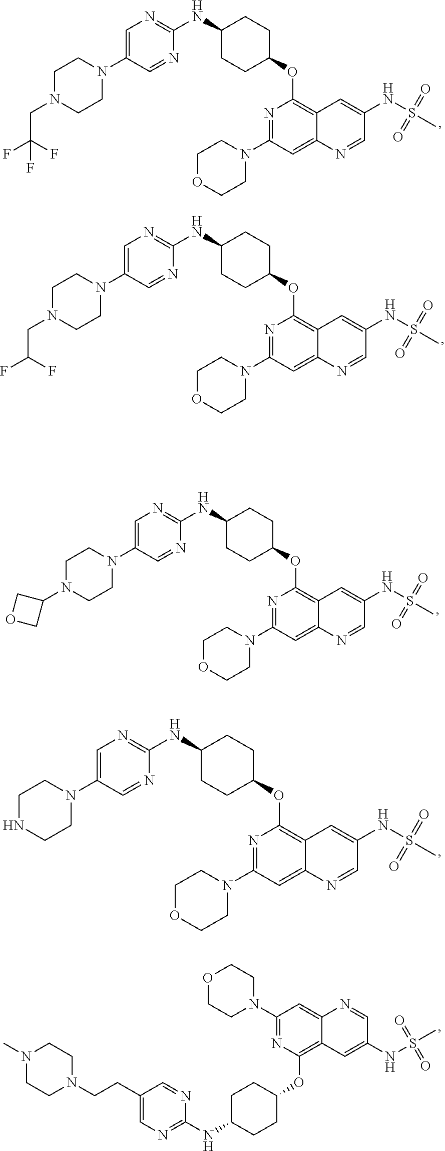

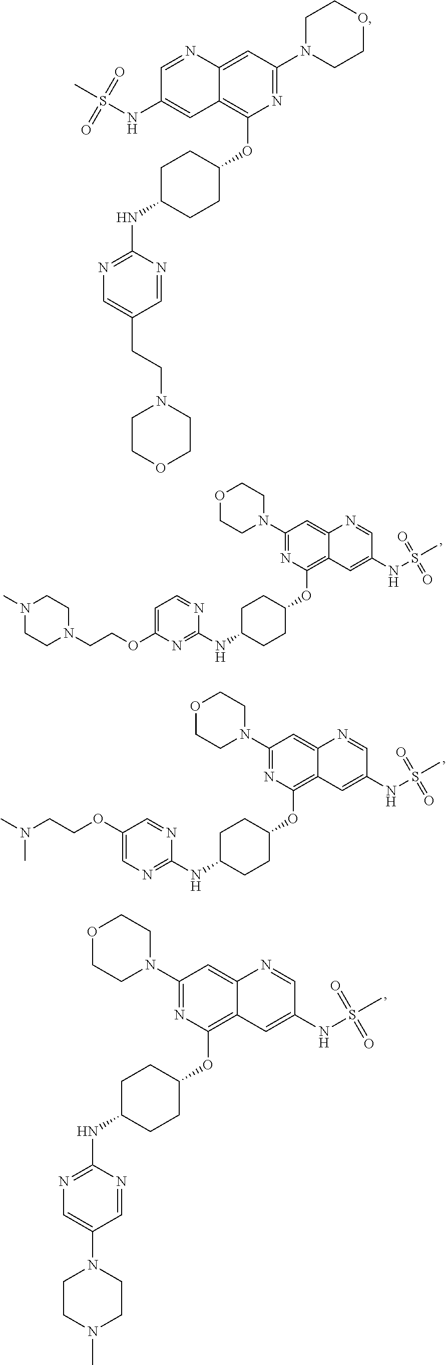

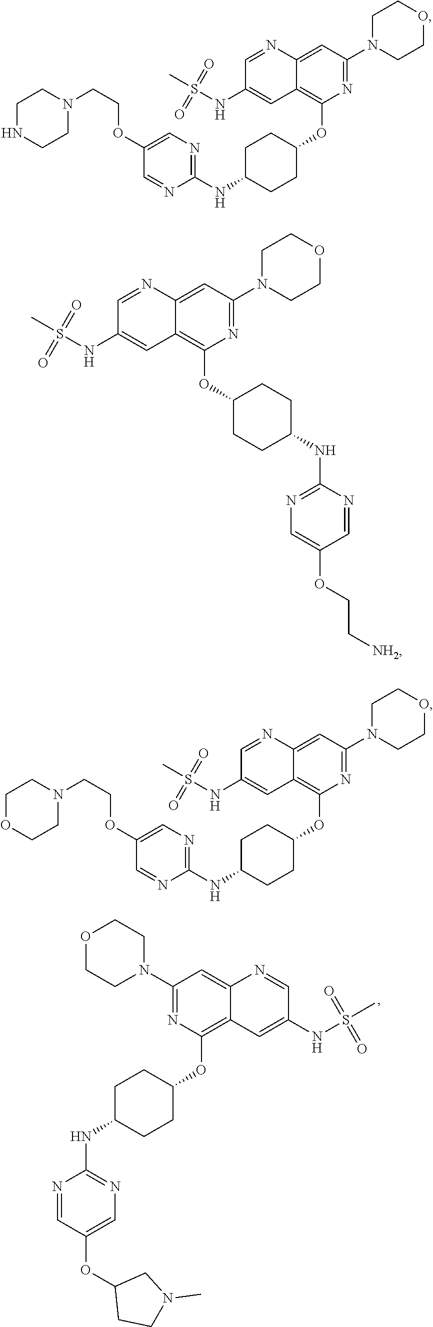 [SMILES: CS(=O)(=O)Nc1cnc2cc(N3CCOCC3)nc(O[C@H]3CC[C@@H](Nc4ncc(OCCN5CCNCC5)cn4)CC3)c2c1 ; CS(=O)(=O)Nc1cnc2cc(N3CCOCC3)nc(O[C@H]3CC[C@@H](Nc4ncc(OCCN)cn4)CC3)c2c1 ; CS(=O)(=O)Nc1cnc2cc(N3CCOCC3)nc(O[C@H]3CC[C@@H](Nc4ncc(OCCN5CCOCC5)cn4)CC3)c2c1 ; CN1CCC(Oc2cnc(N[C@H]3CC[C@@H](Oc4nc(N5CCOCC5)cc5ncc(NS(C)(=O)=O)cc45)CC3)nc2)C1]

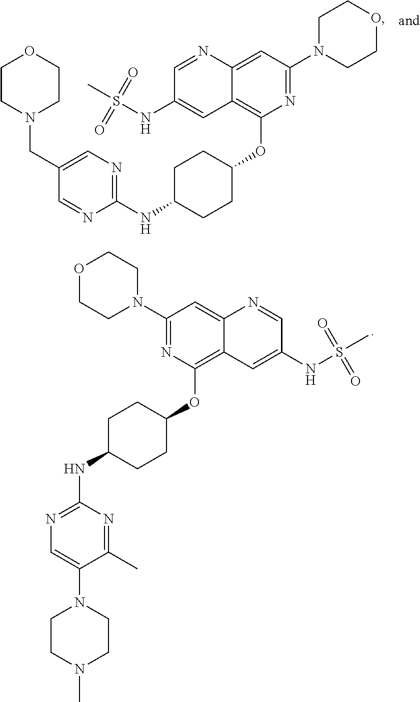

In some embodiments, the compound is selected from:

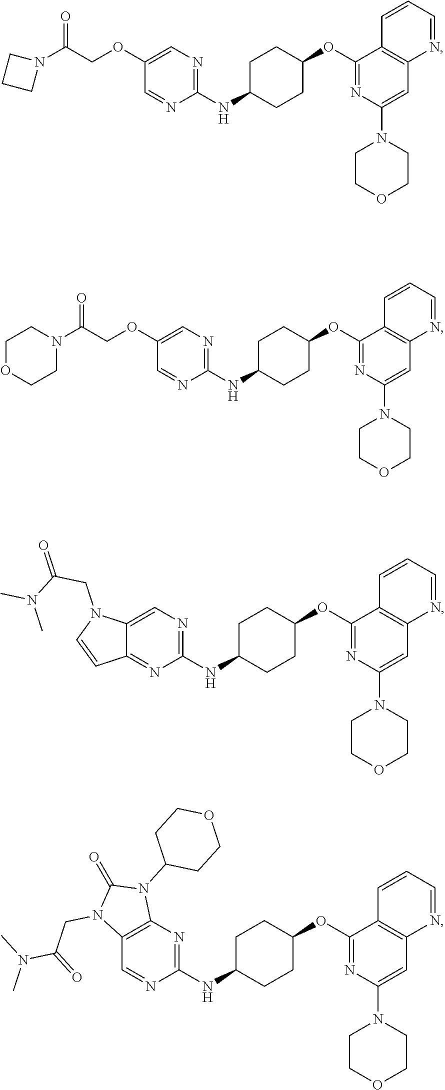 [SMILES: O=C(COc1cnc(N[C@H]2CC[C@@H](Oc3nc(N4CCOCC4)cc4ncccc34)CC2)nc1)N1CCC1 ; O=C(COc1cnc(N[C@H]2CC[C@@H](Oc3nc(N4CCOCC4)cc4ncccc34)CC2)nc1)N1CCOCC1 ; CN(C)C(=O)Cn1ccc2nc(N[C@H]3CC[C@@H](Oc4nc(N5CCOCC5)cc5ncccc45)CC3)ncc21 ; CN(C)C(=O)Cn1c(=O)n(C2CCOCC2)c2nc(N[C@H]3CC[C@@H](Oc4nc(N5CCOCC5)cc5ncccc45)CC3)ncc21]

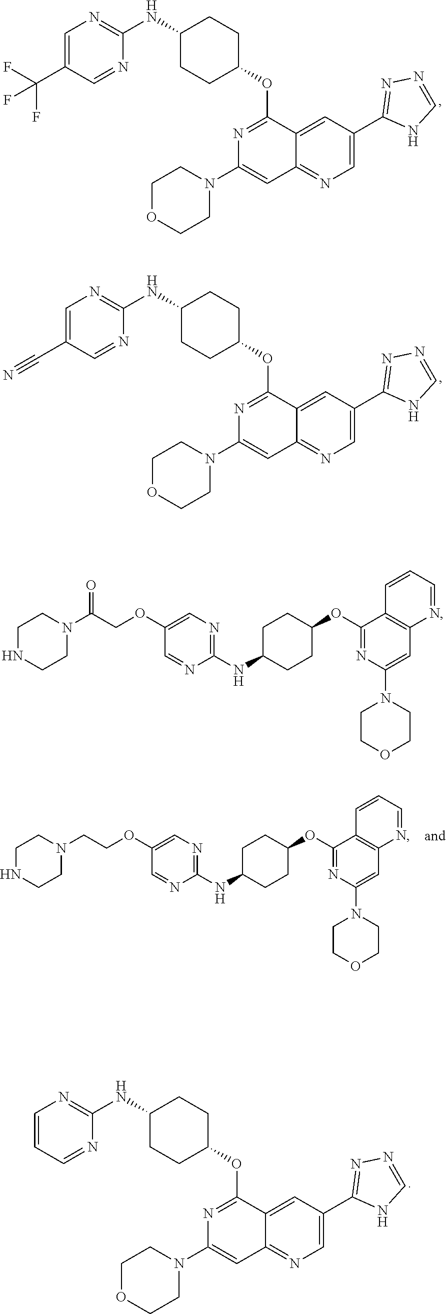

In some embodiments, the compound is selected from:

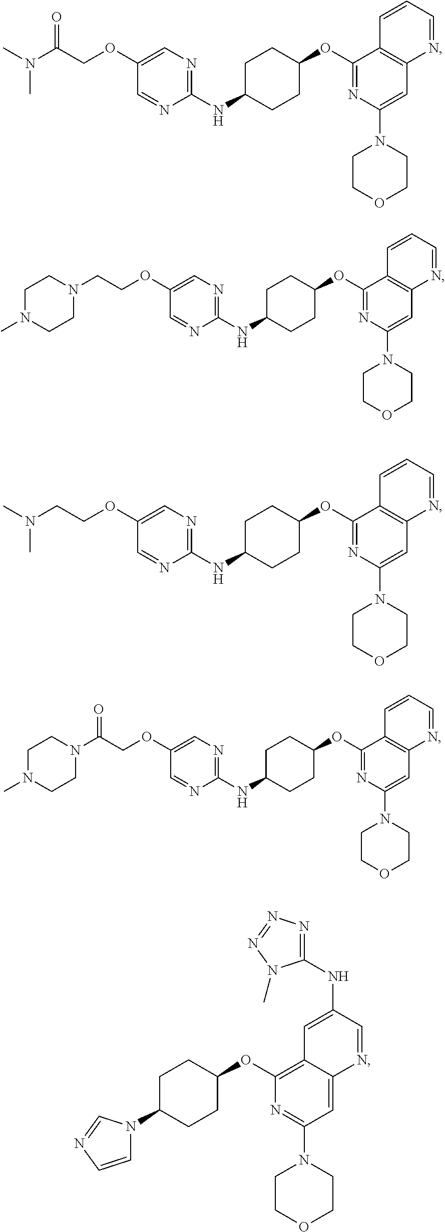 [SMILES: CN(C)C(=O)COc1cnc(N[C@H]2CC[C@@H](Oc3nc(N4CCOCC4)cc4ncccc34)CC2)nc1 ; CN1CCN(CCOc2cnc(N[C@H]3CC[C@@H](Oc4nc(N5CCOCC5)cc5ncccc45)CC3)nc2)CC1 ; CN(C)CCOc1cnc(N[C@H]2CC[C@@H](Oc3nc(N4CCOCC4)cc4ncccc34)CC2)nc1 ; CN1CCN(C(=O)COc2cnc(N[C@H]3CC[C@@H](Oc4nc(N5CCOCC5)cc5ncccc45)CC3)nc2)CC1 ; Cn1nnnc1Nc1cnc2cc(N3CCOCC3)nc(O[C@H]3CC[C@@H](n4ccnc4)CC3)c2c1]

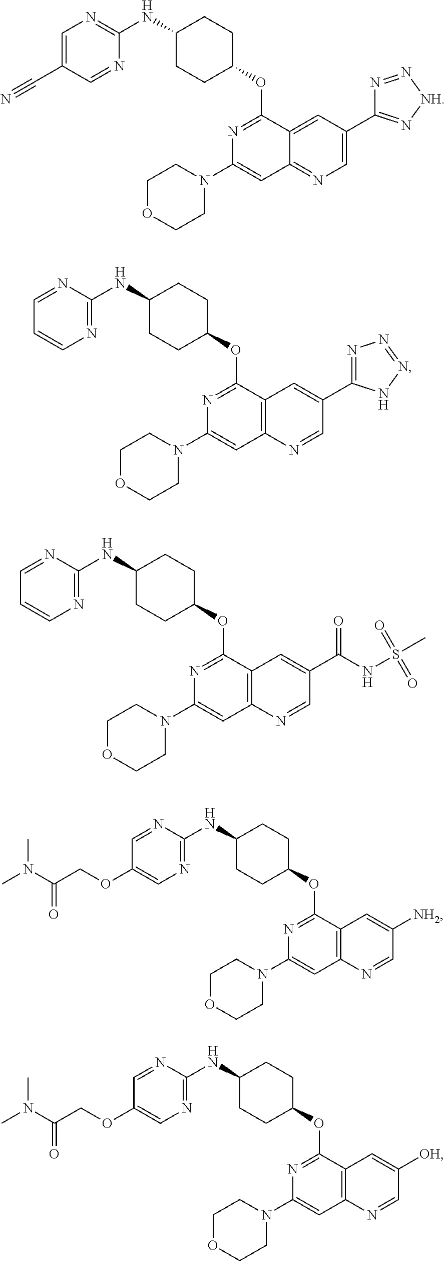

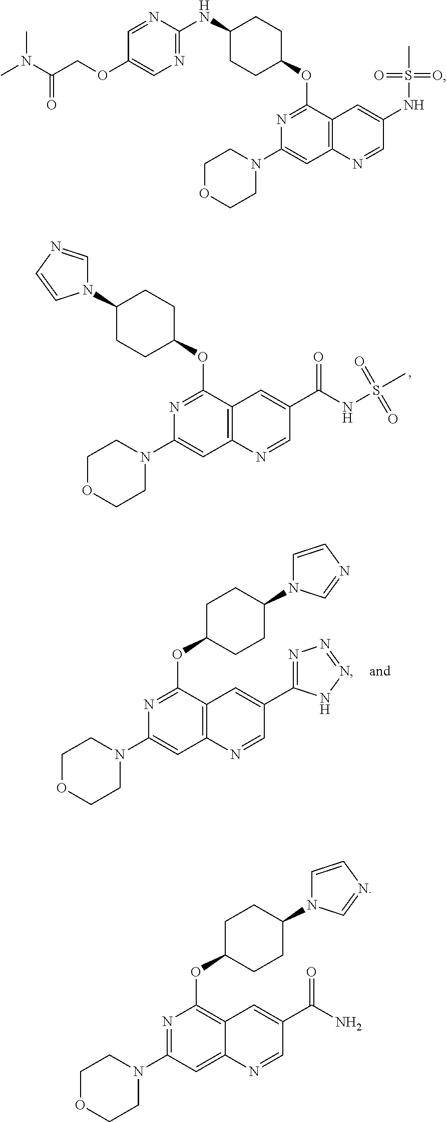 [SMILES: CN(C)C(=O)COc1cnc(N[C@H]2CC[C@@H](Oc3nc(N4CCOCC4)cc4ncc(NS(C)(=O)=O)cc34)CC2)nc1 ; CS(=O)(=O)NC(=O)c1cnc2cc(N3CCOCC3)nc(O[C@H]3CC[C@@H](n4ccnc4)CC3)c2c1 ; c1cn([C@H]2CC[C@@H](Oc3nc(N4CCOCC4)cc4ncc(-c5nnn[nH]5)cc34)CC2)cn1 ; NC(=O)c1cnc2cc(N3CCOCC3)nc(O[C@H]3CC[C@@H](n4ccnc4)CC3)c2c1]

In some embodiments, the compound is a prodrug of a compound of formula (II) or a pharmaceutically acceptable salt thereof.

In some embodiments, the prodrug comprises a trigger moiety that releases the compound of formula (II) under reductive conditions.

In some embodiments, the trigger moiety has a structure selected from:

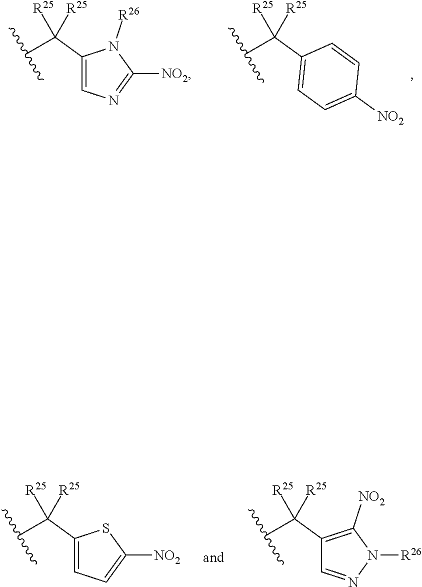

wherein:
each R²⁵ is independently selected from H and C₁-C₆-alkyl; and
R²⁶ is selected from C₁-C₃-alkyl and C₃-C₅-cycloalkyl.

In some embodiments, the compound is selected from:

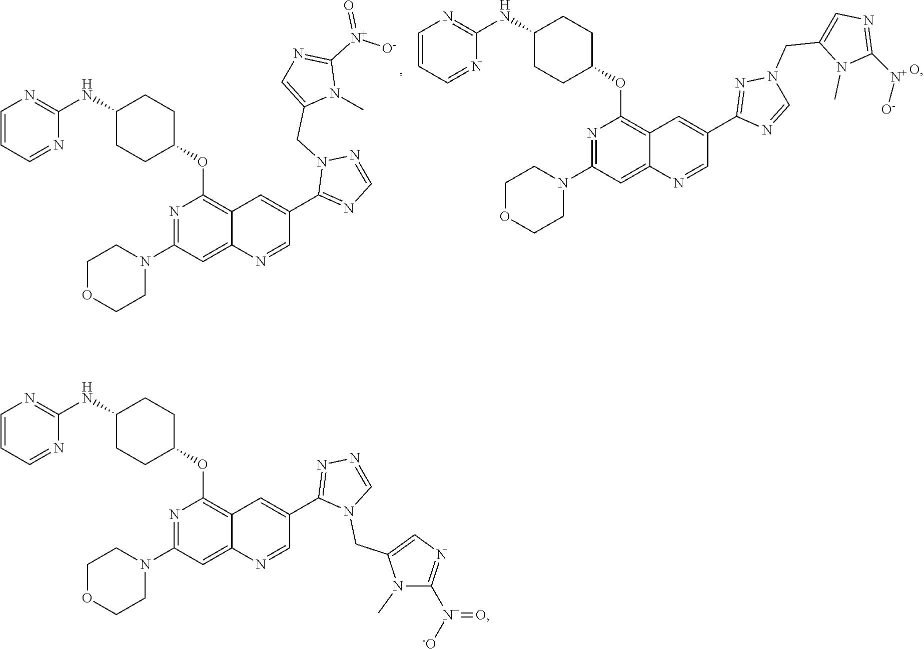

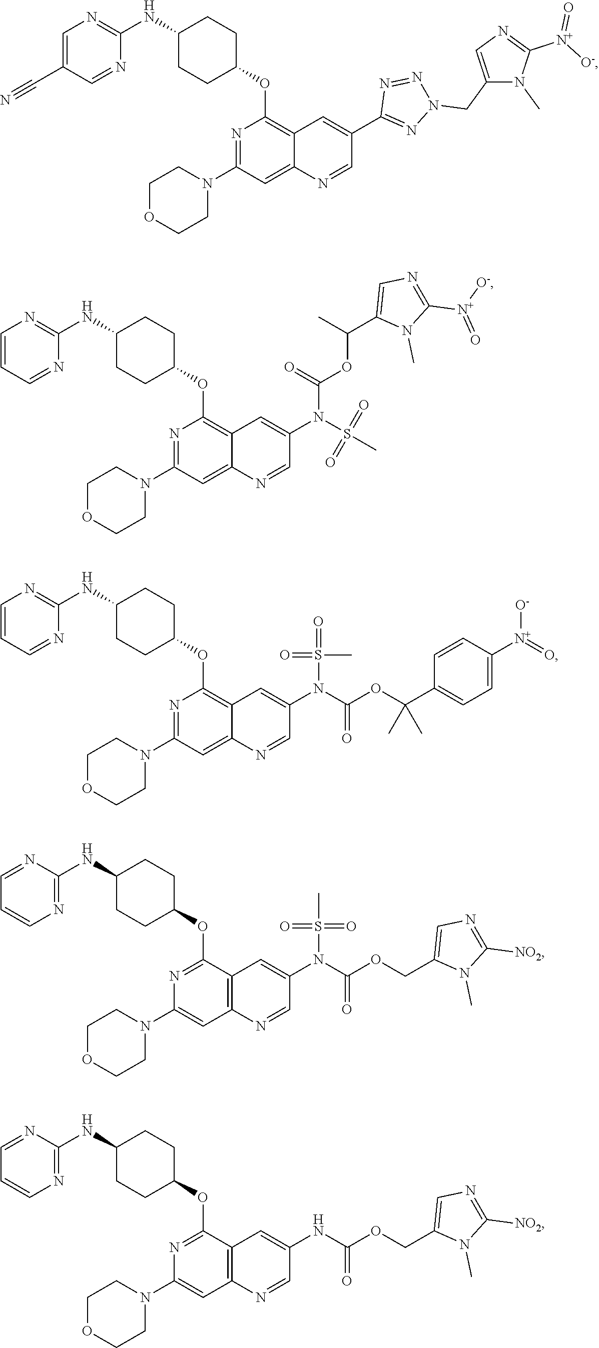

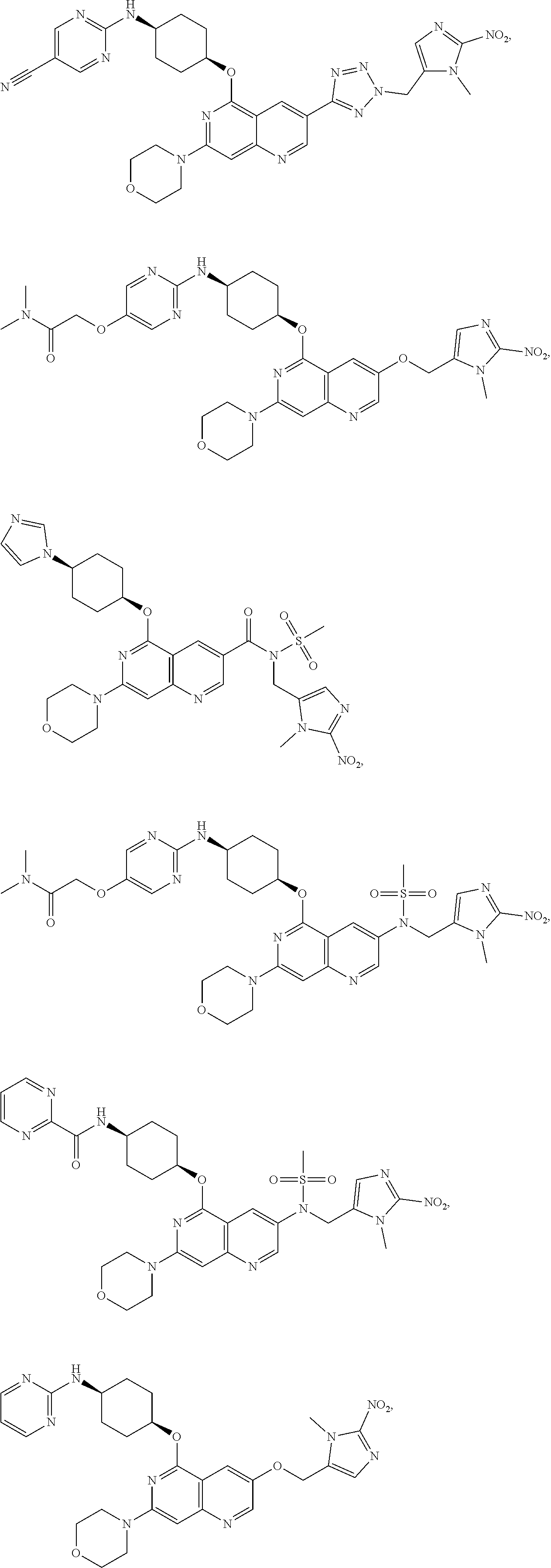

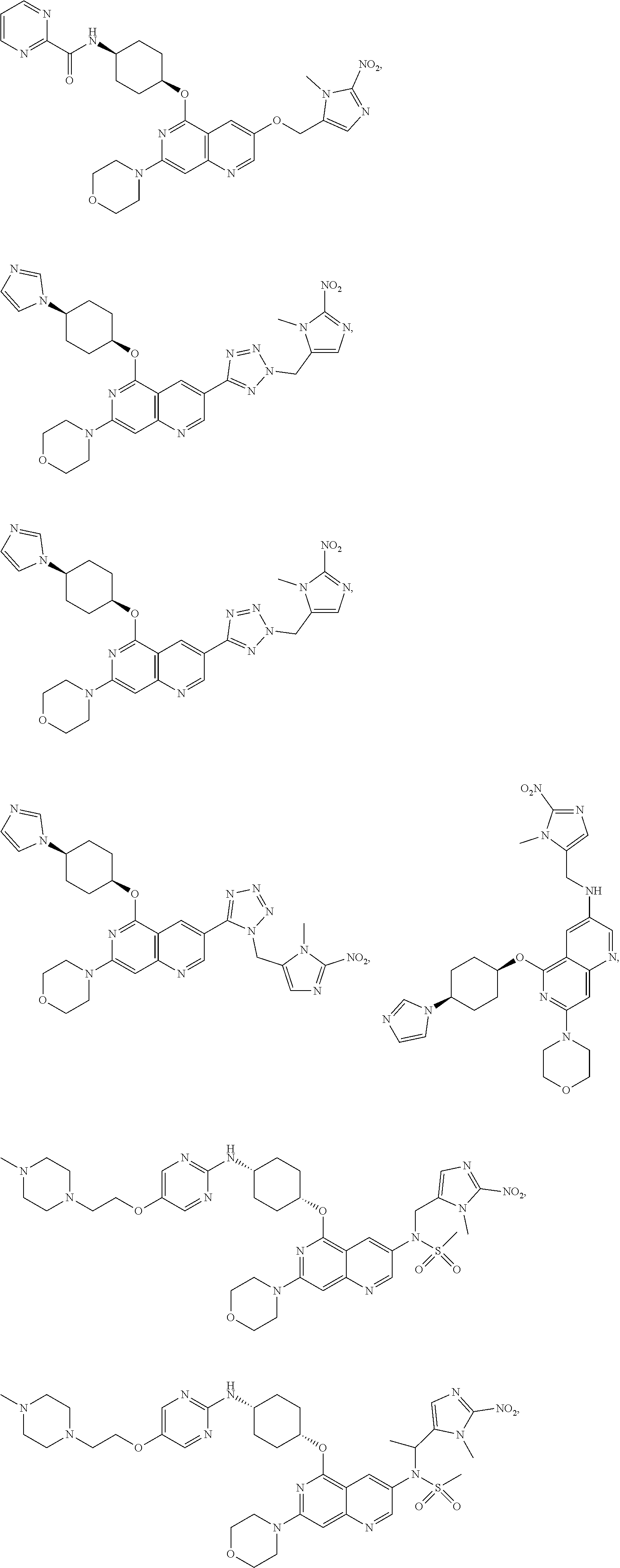

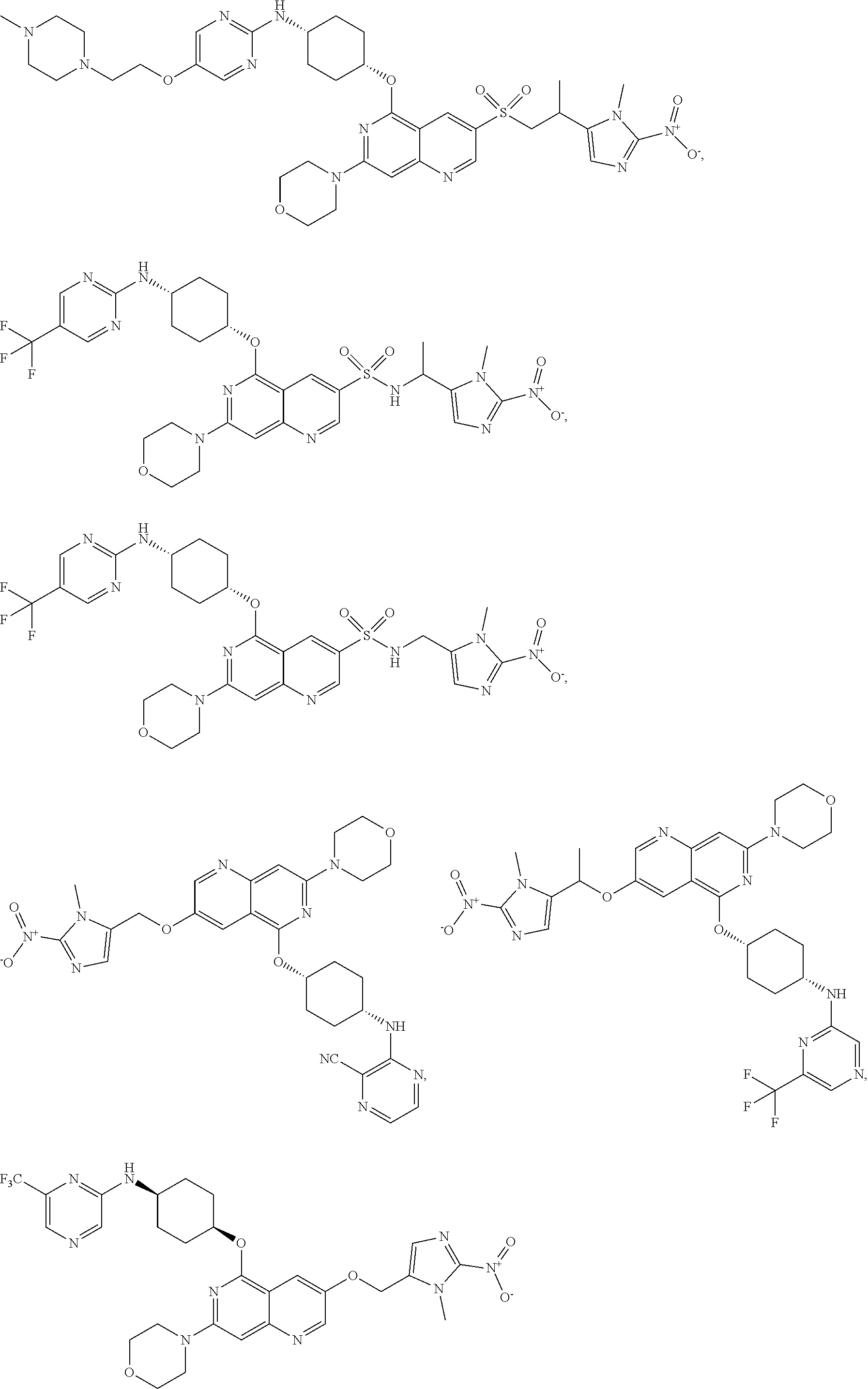

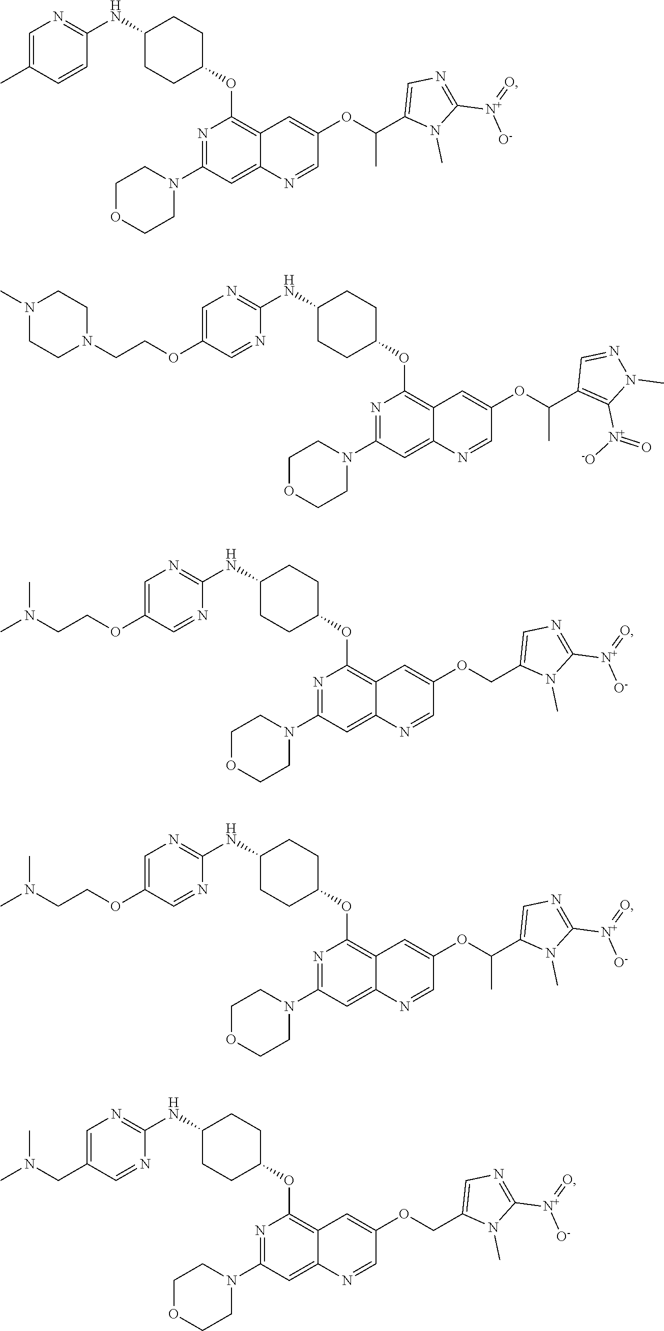

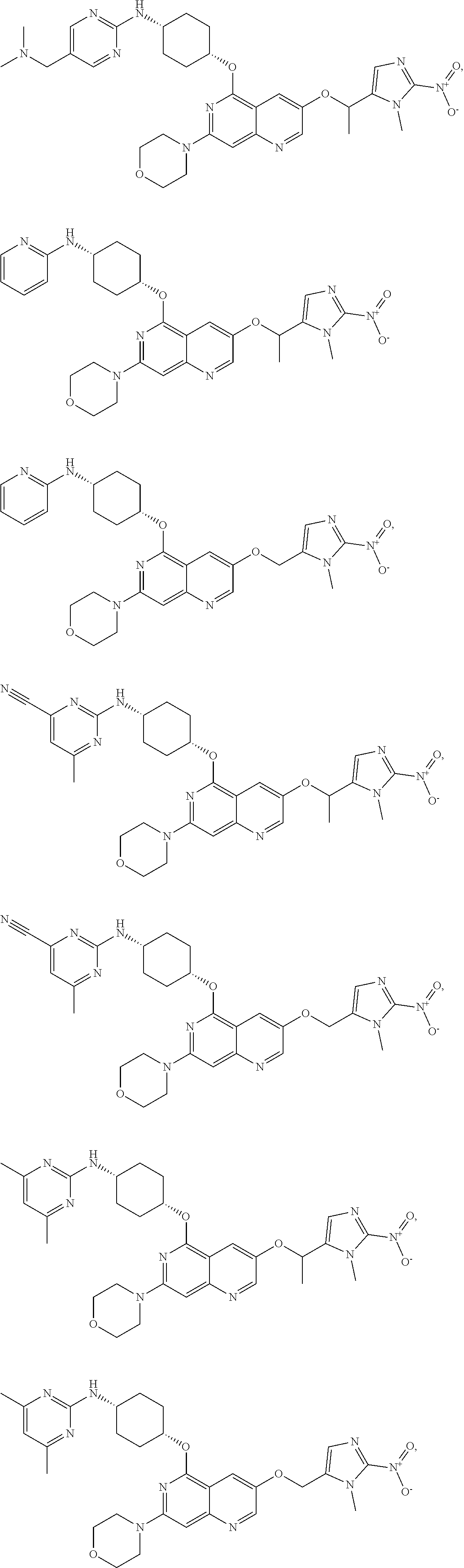

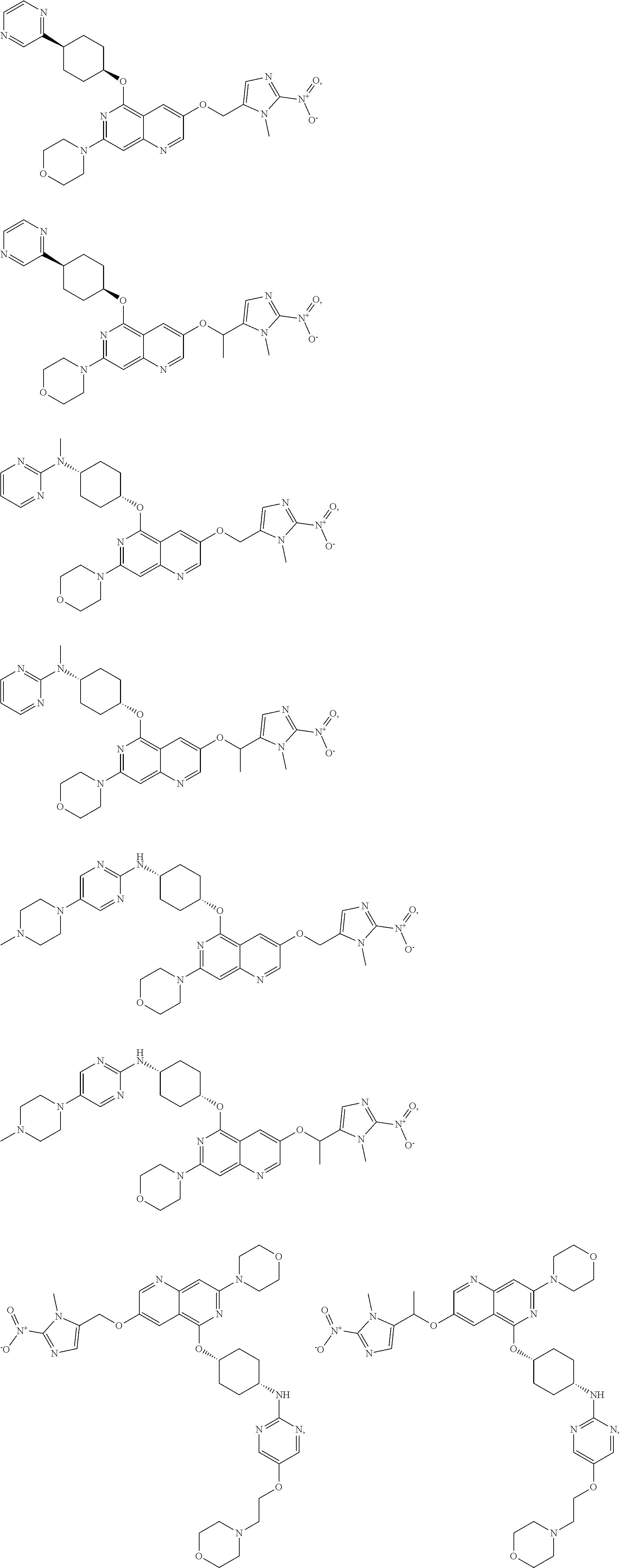

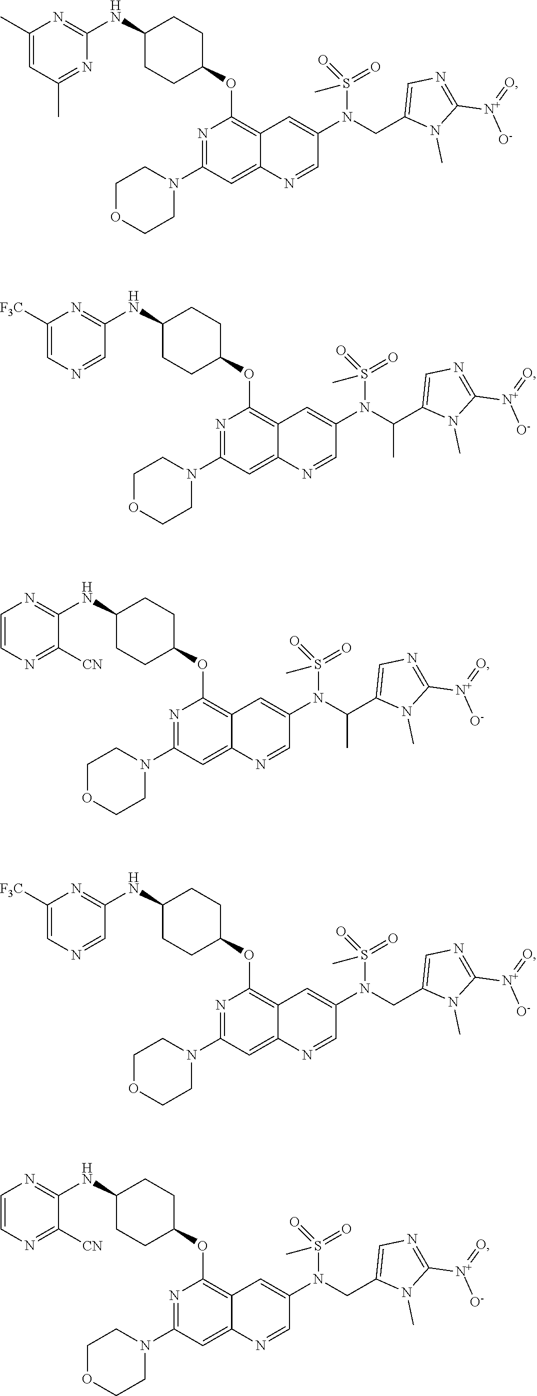

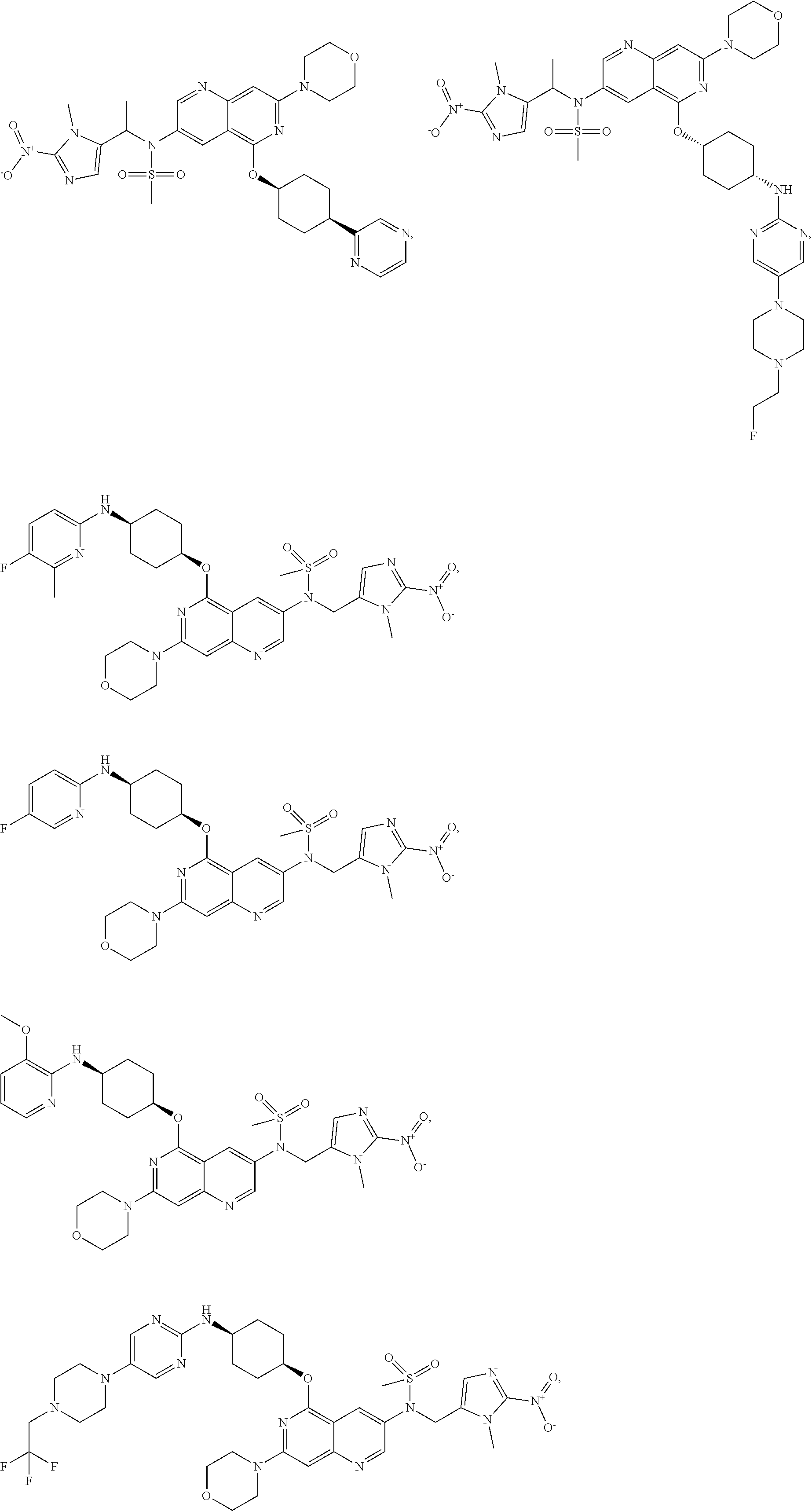

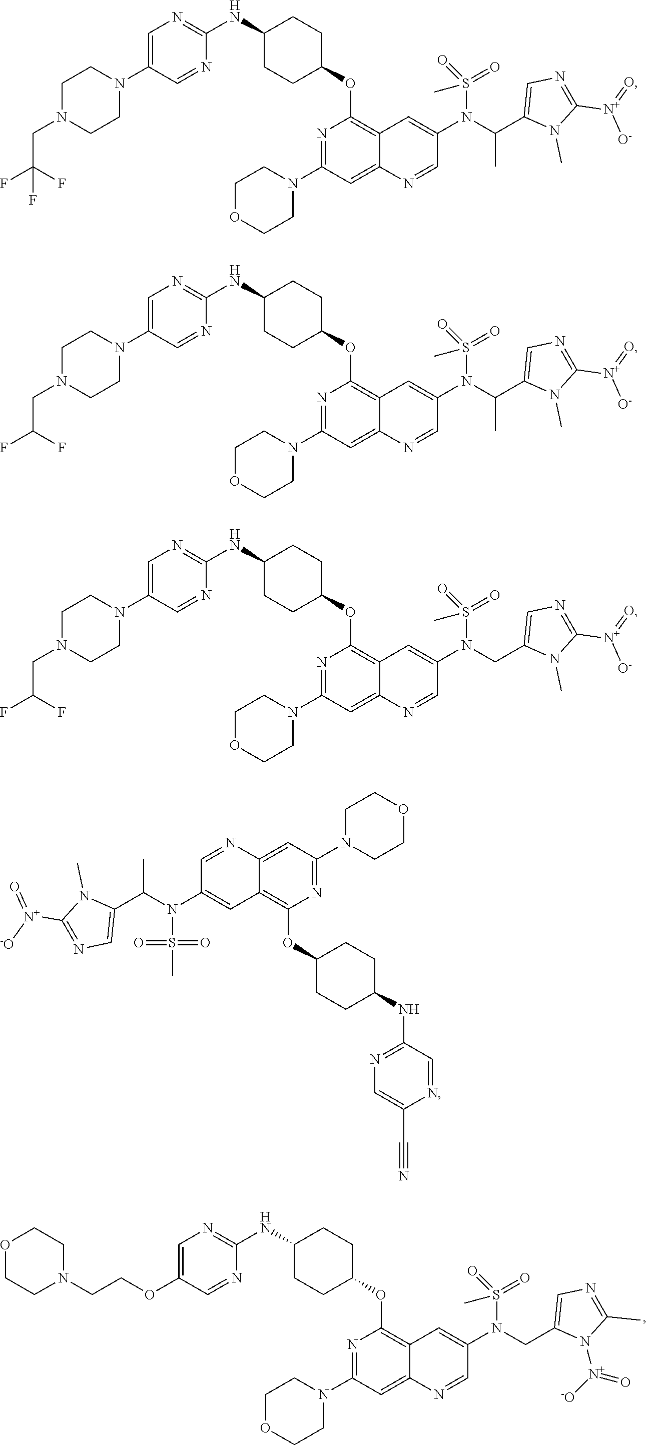

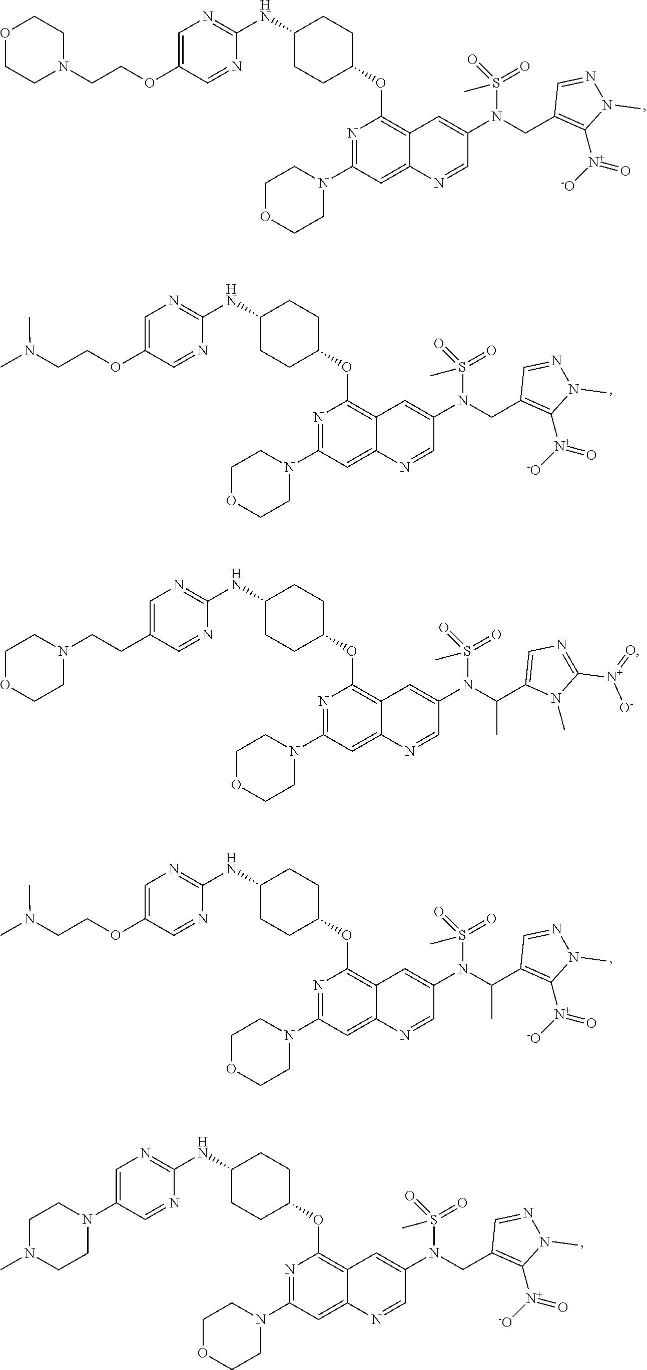

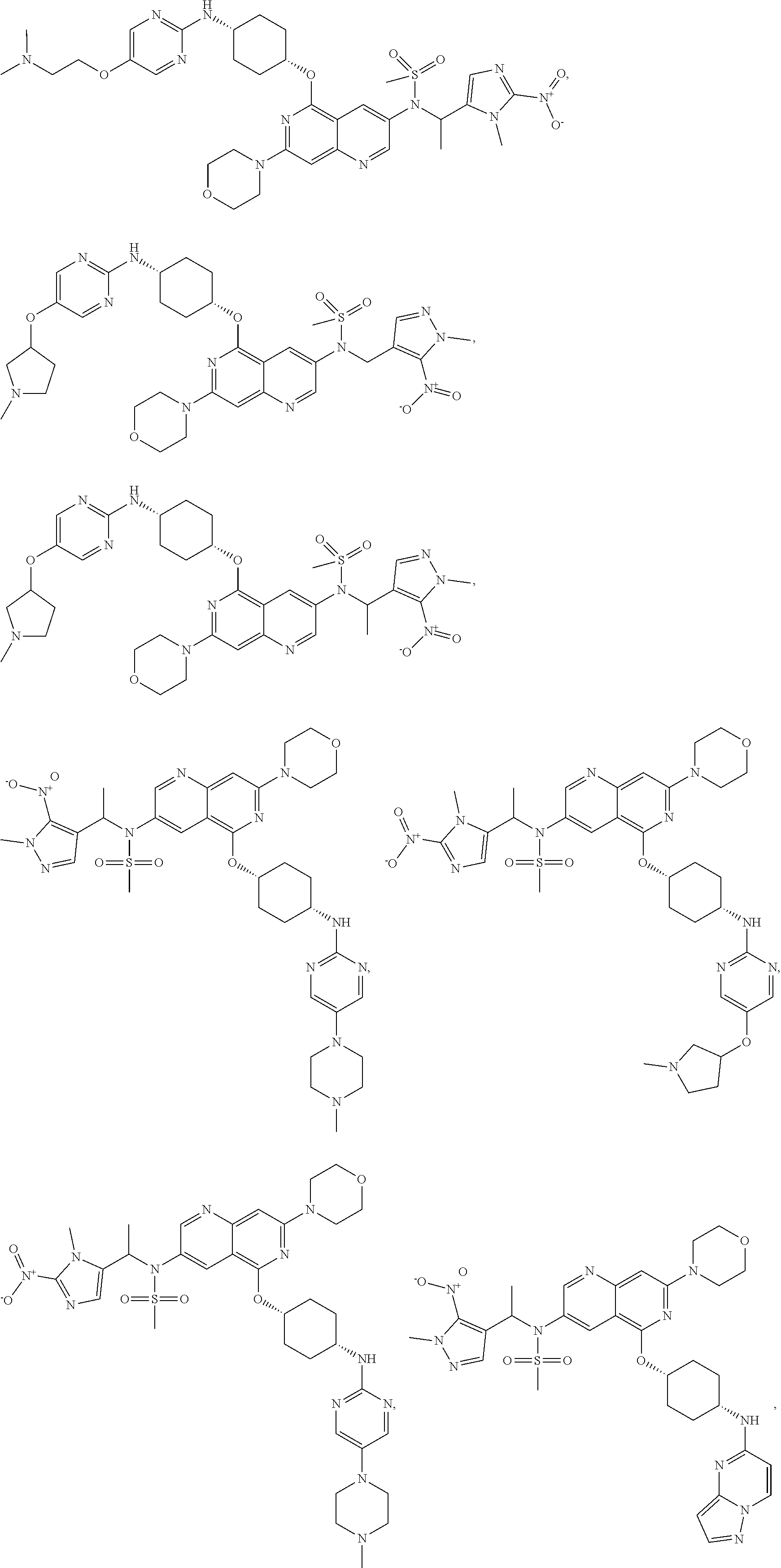

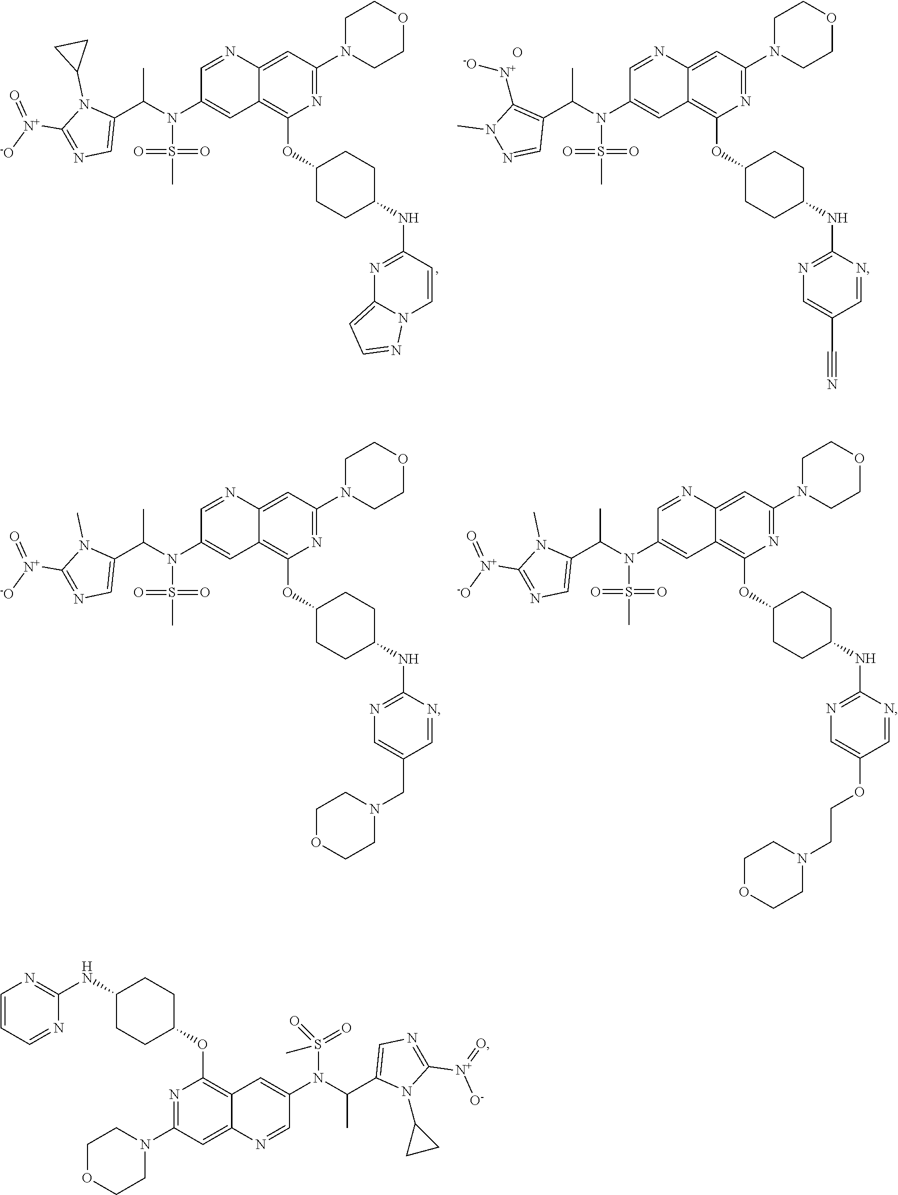

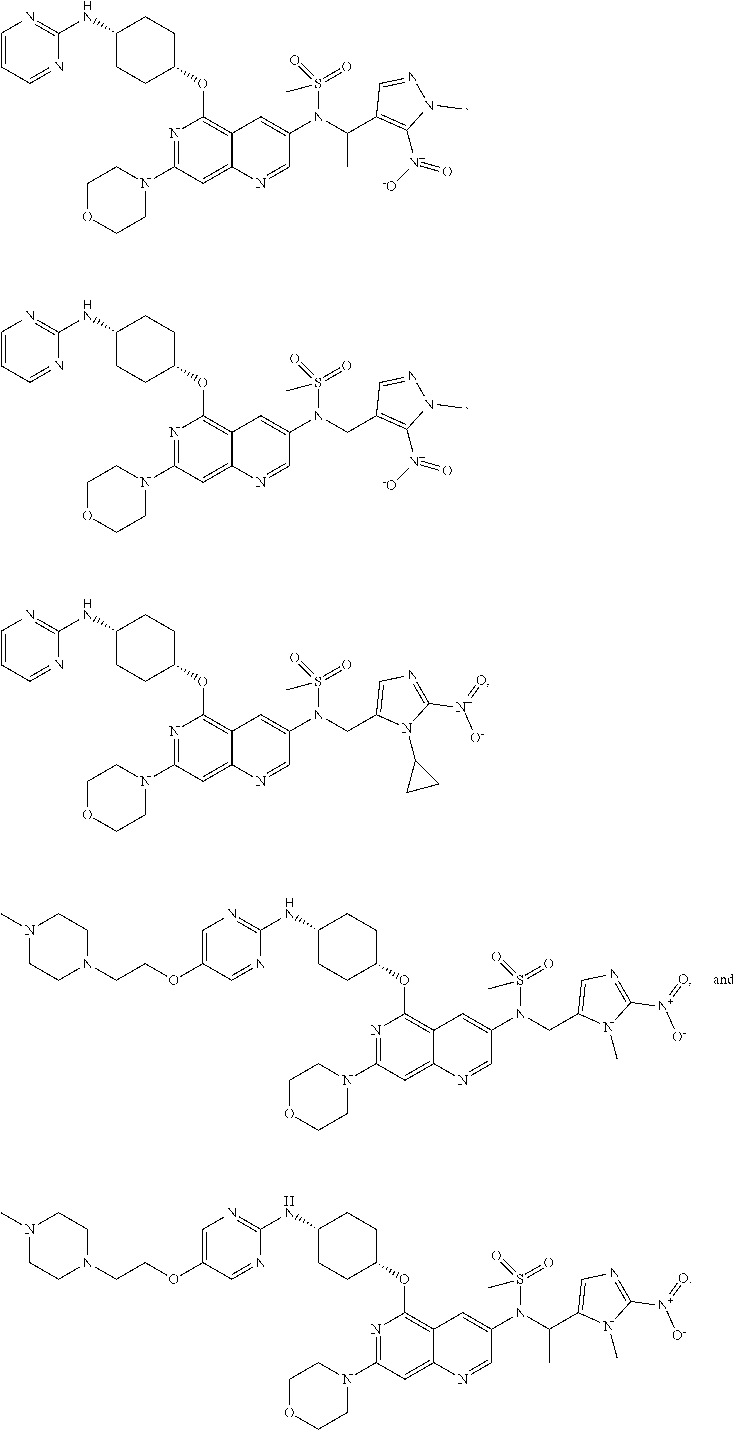

In some embodiments, the compound is selected from:

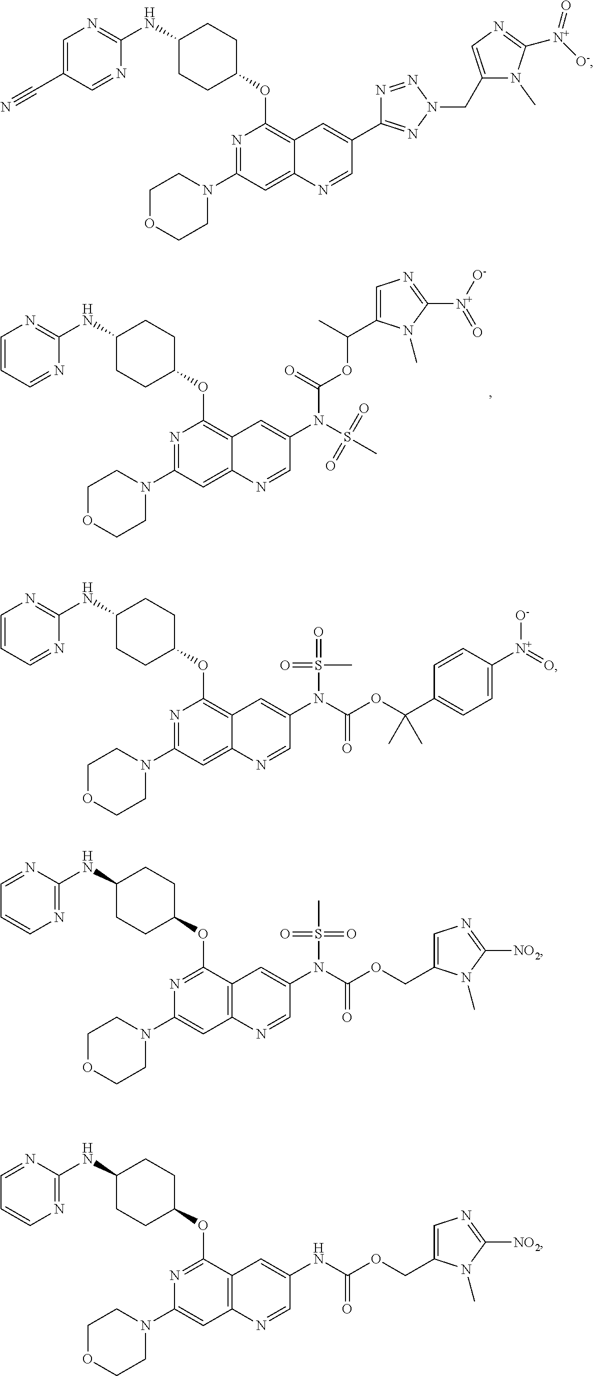

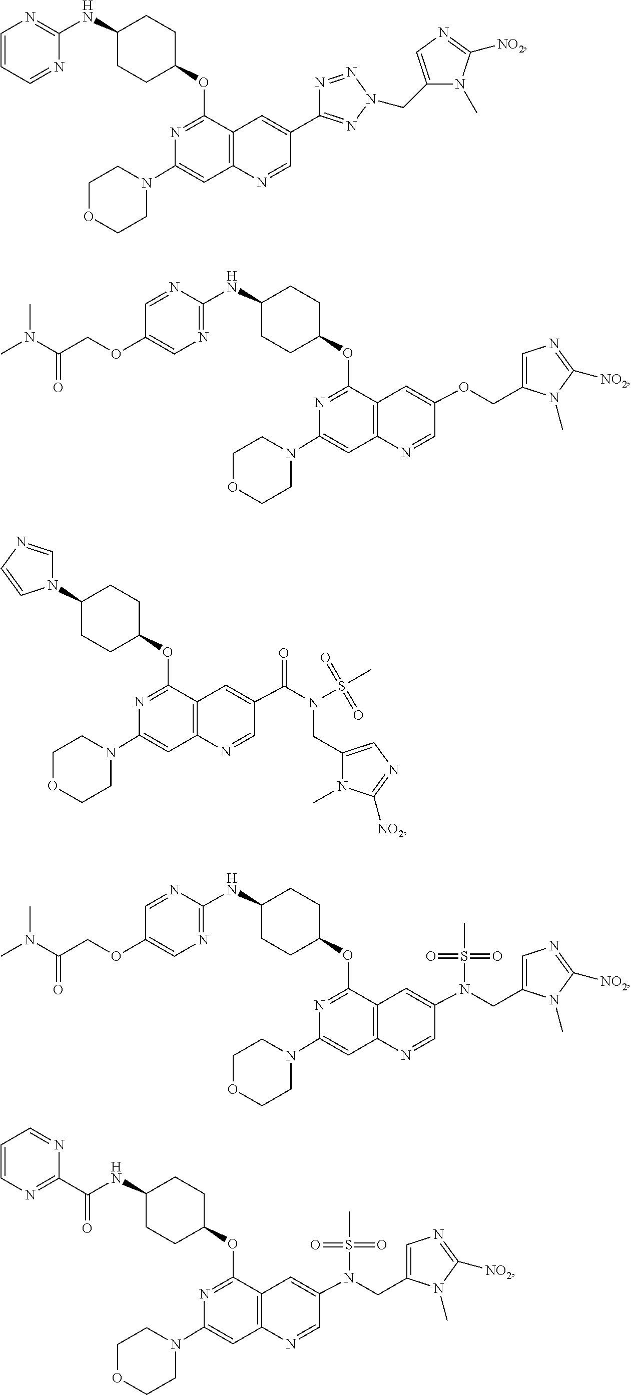 [SMILES: Cn1c(Cn2nnc(-c3cnc4cc(N5CCOCC5)nc(O[C@H]5CC[C@@H](Nc6ncccn6)CC5)c4c3)n2)cnc1[N+](=O)[O-] ; CN(C)C(=O)COc1cnc(N[C@H]2CC[C@@H](Oc3nc(N4CCOCC4)cc4ncc(OCc5cnc([N+](=O)[O-])n5C)cc34)CC2)nc1 ; Cn1c(CN(C(=O)c2cnc3cc(N4CCOCC4)nc(O[C@H]4CC[C@@H](n5ccnc5)CC4)c3c2)S(C)(=O)=O)cnc1[N+](=O)[O-] ; CN(C)C(=O)COc1cnc(N[C@H]2CC[C@@H](Oc3nc(N4CCOCC4)cc4ncc(N(Cc5cnc([N+](=O)[O-])n5C)S(C)(=O)=O)cc34)CC2)nc1 ; Cn1c(CN(c2cnc3cc(N4CCOCC4)nc(O[C@H]4CC[C@@H](NC(=O)c5ncccn5)CC4)c3c2)S(C)(=O)=O)cnc1[N+](=O)[O-]]

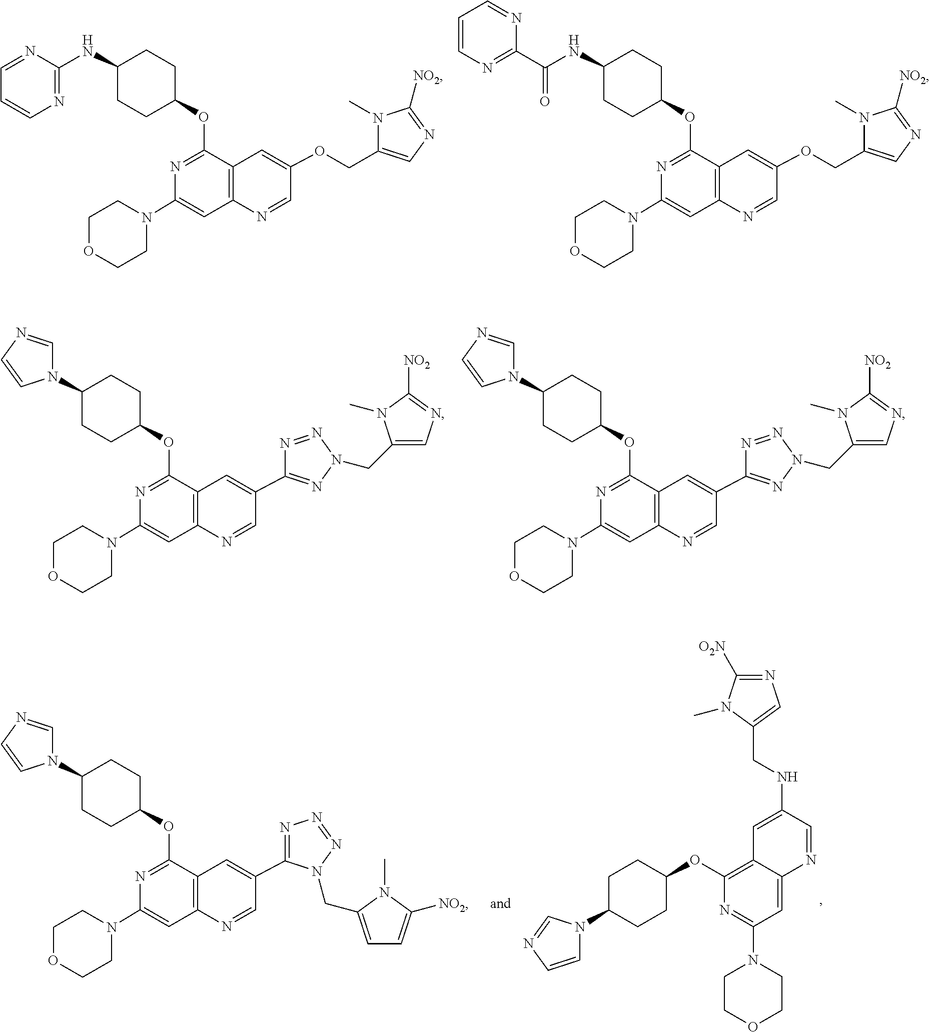

Aspects of the present disclosure include a pharmaceutical composition comprising a compound according to the present disclosure, and a pharmaceutically-acceptable excipient.

Aspects of the present disclosure include a method of inhibiting DNA-PK activity comprising contacting DNA-PK with an effective amount of a compound according to the present disclosure.

Aspects of the present disclosure include a method comprising administering to a subject an effective amount of a compound according to the present disclosure.

Aspects of the present disclosure include a method of treating cancer comprising administering to a subject a therapeutically effective amount of a compound according to the present disclosure.

In some embodiments, the method further comprises treating the subject with radiotherapy and/or a DNA damaging chemotherapeutic agent.

Aspects of the present disclosure include a method of repairing a DNA break in one or more target genomic regions via a homology directed repair (HDR) pathway. In some cases, the method includes administering to one or more cells that comprise one or more target genomic regions, a genome editing system, and a compound according to the present disclosure, wherein the genome editing system interacts with a nucleic acid of the one or more target genomic regions, resulting in a DNA break, and wherein the DNA break is repaired at least in part via a HDR pathway.

Aspects of the present disclosure include a method of modifying expression of one or more genes or proteins. In some cases, the method includes administering to one or more cells that comprise one or more target genomic regions, a genome editing system, and a compound according to the present disclosure, wherein the genome editing system interacts with a nucleic acid of the one or more target genomic regions of a target gene, resulting in editing the one or more target genomic regions, and wherein the edit modifies expression of a downstream gene and/or protein associated with the target gene.

In some embodiments, the efficacy of the repair of the DNA break at the one or more target genomic regions via a HDR pathway is increased as compared to a cell in the absence of the compound.

In some embodiments, the efficacy editing the one or more target genomic regions is increased as compared to a cell in the absence of the compound.

In some embodiments, the genome editing system is selected from a meganuclease based system, a zinc finger nuclease (ZFN) based system, a Transcription Activator-Like Effector-based Nuclease (TALEN) system, a CRISPR-based system, and a NgAgo-based system.

In some embodiments, the genome editing system is a CRISPR-based system.

In some embodiments, the CRISPR-based system is a CRISPR-Cas system or a CRISPR-Cpf system.

### Definitions

The following terms have the following meanings unless otherwise indicated. Any undefined terms have their art recognized meanings.

The term Cr-Cy refers to a group with x to y carbon atoms.

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain (except the C₁ carbon atom) have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein Ra and R^{b} may be the same or different and are chosen from hydrogen, optionally substituted alkyl, cycloalkyl, alkenyl, cycloalkenyl, alkynyl, aryl, heteroaryl and heterocyclic.

The term “haloalkyl” refers to a hydrocarbon chain substituted with at least one halogen atom independently chosen at each occurrence, for example fluorine, chlorine, bromine and iodine. The halogen atom may be present at any position on the hydrocarbon chain. For example, C₁-C₆-haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl.

The term “heteroalkyl” refers to an alkyl group as defined herein wherein one or more carbon atoms in the alkyl chain (except the C₁ carbon atom) have been replaced with a heteroatom such as —O—, —N—, —S—, —S(O)ₙ— (where n is 0 to 2), or —NR— (where R is hydrogen or alkyl).

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like, where R¹⁰ is chosen from chosen from hydrogen, optionally substituted alkyl, cycloalkyl, alkenyl, cycloalkenyl, alkynyl, aryl, heteroaryl and heterocyclic. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NR″— where R′ is alkyl group as defined herein and R″ is alkylene, alkenylene or alkynylene group as defined herein.

The term “alkaryl” or “aralkyl” refers to the groups-alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, heterocycloalkyl-O—, cycloalkenyl-O—, and alkynyl-O—, where alkenyl, cycloalkyl, heterocycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group (e.g., fluorine, chlorine, bromine, iodine). Examples of such haloalkyl groups include, but are not limited to, chloromethyl, fluoromethyl, trifluoromethyl, chloroethyl (e.g. 1-chloromethyl and 2-chloroethyl), trichloroethyl (e.g. 1,2,2-trichloroethyl, 2,2,2-trichloroethyl), fluoroethyl (e.g. 1-fluoromethyl and 2-fluoroethyl), trifluoroethyl (e.g. 1,2,2-trifluoroethyl and 2,2,2-trifluoroethyl), chloropropyl, trichloropropyl, fluoropropyl, trifluoropropyl, and the like.

The term “alkylalkoxy” refers to the groups-alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl, and —SO2-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Acylamino” refers to the groups —NR²⁰C(O)alkyl, —NR²⁰C(O)substituted alkyl, NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R²³ are independently selected from hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “alkoxycarbonylamino” refers to the group —NR^{d}C(O)OR^{d} where each R^{d} is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R²¹ and R²² independently are selected from hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Sulfonylamino” refers to the group —NR²¹SO₂R²², wherein R²¹ and R²² independently are selected from hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 5 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NR^{m}R^{m} where each R^{m} is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R^{m} is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O— substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl, bicyclo[2.1.1]hexane, bicyclo[1.1.1]pentane, and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Carbocycle” refers to non-aromatic or aromatic cyclic groups, such as cycloalkyl, cycloalkenyl, cycloalkynyl, and aryl groups as defined herein. A carbocycle group may be unsubstituted or substituted as defined herein.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic. To satisfy valence requirements, any heteroatoms in such heteroaryl rings may or may not be bonded to H or a substituent group, e.g., an alkyl group or other substituent as described herein. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In some embodiments, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl, 7H-pyrrolo[2,3-d]pyrimidinyl, 8,9-dihydro-7H-purinyl, pyrazolo[1,5-a]pyrimidinyl, imidazo[1,2-b]pyridazinyl, [1,2,4]triazolo[1,5-a]pyridinyl, furo[3,2-d]pyrimidinyl, furo[2,3-d]pyrimidinyl, thieno[3,2-d]pyrimidinyl, and the like.

Examples of heteroaryl groups comprising at least one nitrogen in a ring position include pyrrolyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, indolyl, isoindolyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl and pteridinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups. Examples of six membered heteroaryl groups include but are not limited to pyridyl, pyrazinyl, pyridazinyl, pyrimidinyl and triazinyl. Particular examples of bicyclic heteroaryl groups containing a six membered ring fused to a five membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl, pyrrolopyridine, and pyrazolopyridinyl groups. Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “heteroaralkyl” refers to the groups-alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from nitrogen, sulfur, or oxygen, where, in fused ring systems, one or more of the rings can be cycloalkyl, heterocyclyl, aryl, or heteroaryl, provided that the point of attachment is through the non-aromatic ring. Fused ring systems include compounds where two rings share two adjacent atoms. In fused heterocycle systems one or both of the two fused rings can be heterocyclyl. In certain embodiments, the nitrogen and/or sulfur atom(s) of the heterocyclic group are optionally oxidized to provide for the N-oxide, —S(O)—, or —SO₂— moieties. To satisfy valence requirements, any heteroatoms in such heterocyclic rings may or may not be bonded to one or more H or one or more substituent group(s), e.g., an alkyl group or other substituent as described herein.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, 1,2,3,4-tetrahydroquinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, 3,4-dihydro-1,4-benzoxazine, thiomorpholinyl (also referred to as thiamorpholinyl), 1,1-dioxothiomorpholinyl, piperidinyl, pyrrolidine, tetrahydrofuranyl, and the like.

Examples of heterocyclic groups include cyclic ethers such as oxiranyl, oxetanyl, tetrahydrofuranyl, dioxanyl, and substituted cyclic ethers. Heterocycles comprising at least one nitrogen in a ring position include, for example, azetidinyl, pyrrolidinyl, piperidinyl, piperazinyl, morpholinyl, thiomorpholinyl, tetrahydrotriazinyl, tetrahydropyrazolyl, tetrahydropyridinyl, homopiperidinyl, homopiperazinyl, 3,8-diaza-bicyclo[3.2.1]octanyl, 8-aza-bicyclo[3.2.1]octanyl, 2,5-Diazabicyclo[2.2.1]heptanyl and the like. Typical sulfur containing heterocycles include tetrahydrothienyl, dihydro-1,3-dithiol, tetrahydro-2H-thiopyran, and hexahydrothiepine. Other heterocycles include dihydro oxathiolyl, tetrahydro oxazolyl, tetrahydro-oxadiazolyl, tetrahydrodioxazolyl, tetrahydrooxathiazolyl, hexahydrotriazinyl, tetrahydro oxazinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O), for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. For example, in some instances, reference to piperidino or morpholino refers to a piperidin-1-yl or morpholin-4-yl ring that is linked via the ring nitrogen.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, OSO₂-substituted alkyl, OSO₂-alkenyl, OSO₂-substituted alkenyl, OSO₂-cycloalkyl, OSO₂-substituted cycloalkyl, OSO₂-cycloalkenyl, OSO₂-substituted cylcoalkenyl, OSO₂-aryl, OSO₂-substituted aryl, OSO₂-heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O—)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸OR⁸⁰, —C(NR⁷⁰)NR⁸⁰OR⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of 0, N and S, of which N may have —H or C₁-C₃ alkyl substitution; and each M⁺is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸OR⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂ O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸OR⁸⁰, —C(NR⁷⁰)NR⁸OR⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰OR⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

[symbol]

A bond terminating in a “ [symbol] ” represents that the bond is connected to another atom that is not shown in the structure. A bond terminating inside a cyclic structure and not terminating at an atom of the ring structure represents that the bond may be connected to any of the atoms in the ring structure where allowed by valency.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N′-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

“Pharmaceutically effective amount” and “therapeutically effective amount” refer to an amount of a compound sufficient to treat a specified disorder or disease or one or more of its symptoms and/or to prevent the occurrence of the disease or disorder. In reference to tumorigenic proliferative disorders, a pharmaceutically or therapeutically effective amount comprises an amount sufficient to, among other things, cause the tumor to shrink or decrease the growth rate of the tumor.

By “treating” or “treatment” is meant that at least an amelioration of the symptoms associated with the condition afflicting the subject is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the subject no longer suffers from the condition, or at least the symptoms that characterize the condition. Thus treatment includes: (i) prevention, that is, reducing the risk of development of clinical symptoms, including causing the clinical symptoms not to develop, e.g., preventing disease progression to a harmful state or prophylactic treatment of a subject; (ii) inhibition, that is, arresting the development or further development of clinical symptoms, e.g., mitigating or completely inhibiting an active disease; and/or (iii) relief, that is, causing the regression of clinical symptoms or alleviating one or more symptoms of the disease or medical condition in the subject.

The terms “polypeptide,” “peptide,” and “protein” are used interchangeably herein to refer to a polymeric form of amino acids of any length. Unless specifically indicated otherwise, “polypeptide,” “peptide,” and “protein” can include genetically coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, proteins which contain at least one N-terminal methionine residue (e.g., to facilitate production in a recombinant host cell); immunologically tagged proteins; and the like.

“Native amino acid sequence” or “parent amino acid sequence” are used interchangeably herein to refer to the amino acid sequence of a polypeptide prior to modification to include a modified amino acid residue.

The terms “amino acid analog,” “unnatural amino acid,” and the like may be used interchangeably, and include amino acid-like compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as C₁ or Br, etc.), deletion of a group, substitution of a covalent bond (single bond for double bond, etc.), or combinations thereof. For example, amino acid analogs may include α-hydroxy acids, and α-amino acids, and the like.

The terms “amino acid side chain” or “side chain of an amino acid” and the like may be used to refer to the substituent attached to the α-carbon of an amino acid residue, including natural amino acids, unnatural amino acids, and amino acid analogs. An amino acid side chain can also include an amino acid side chain as described in the context of the modified amino acids and/or conjugates described herein.

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, at least 75% free, at least 80% free, at least 85% free, at least 90% free, at least 95% free, at least 98% free, or more than 98% free, from other components with which it is naturally associated.

The term “physiological conditions” is meant to encompass those conditions compatible with living cells, e.g., predominantly aqueous conditions of a temperature, pH, salinity, etc. that are compatible with living cells.

As used herein, the term “chronic administration” refers to repeated administration of a compound to a subject. In such treatment, the compound can be administered at least once a week, such as at least once a day, or at least twice or three times a day for a period of at least one month, such as for example five months or more.

As used herein, the term “cysteine protease” refers to a protease having a nucleophilic thiol group in the active site. Cysteine proteases from different organisms can have significantly different cleavage sites. In many RNA class IV viruses, such as coronaviruses, rhinovirus, coxackieviruses and noroviruses, a well-conserved consensus sequence for the 3-chymotrypsin protease (3CP) and 3-chymotrypsin-like protease (3CLP) are observed. For these viruses, this is the main protease (also known as Mpro) responsible for cleaving the polyprotein generated from translation of the viral genome, which liberates the active viral proteins that are critical for viral replication. As this is not a host protease responsible for other critical functions, producing drugs that are highly selective for this viral protease will allow viral replication to be stopped and minimize toxicity for the host. To obtain sufficient inhibition of the protease activity and selectivity over other protease classes, the catalytic mechanism must also be considered in inhibitor design. For cysteine proteases, forming a covalent bond to the catalytic sulfur will ablate activity as it is vital to the cleavage mechanism; however, in some instances, excessive reactivity of the electrophile will also react with serine proteases, other cysteine proteases and other thiols resulting in toxicity. A moiety that forms the covalent bond to the sulfur in the inhibitor is termed the warhead.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present invention and are disclosed herein just as if each and every such sub-combination was individually and explicitly disclosed herein.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### BRIEF DESCRIPTION OF THE DRAWINGS

FIG. 1 shows the design of the two-in-one gRNA/CRISPR-Cas9 dual plasmid vector.

FIG. 2 shows the design of donor template plasmid vector.

FIG. 3 shows the cell line, and the targeted polynucleotide region, used in the traffic light reporter assay for monitoring HDR efficiency.

FIG. 4 shows the experiment workflow used in the traffic light reporter assay for monitoring HDR efficiency.

FIG. 5 shows Plasma concentrations across time following administration of compound 97 as a single bolus via IV and PO gavage to mice. n=3/timepoint. Timepoints include 5 min (IV only), 15 min, 30 min, 1 h, 2 h, 4 h, 6 h, 8 h, 10 h, 16 h (PO only), 24 h (PO only).

FIG. 6 shows Plasma concentration across time following administration of compound 61 as a single bolus via IV and PO gavage to mice. n=3/timepoint. Timepoints include 5 min (IV only), 15 min, 30 min, 1 h, 2 h, 4 h, 6 h, 8 h, 10 h, 16 h (PO only), 24 h (PO only).

FIG. 7 shows Plasma concentration across time following administration of compound 86 as a single bolus via IV and PO gavage to mice. n=3/timepoint. Timepoints include 5 min (IV only), 15 min, 30 min, 1 h, 2 h, 4 h, 6 h, 8 h, 10 h, 16 h (PO only), 24 h (PO only).

FIG. 8 show Plasma concentration across time following administration of compound 75 as a single bolus via IV and PO gavage to mice. n=3/timepoint. Timepoints include 5 min (IV only), 15 min, 30 min, 1 h, 2 h, 4 h, 6 h, 8 h, 10 h, 16 h (PO only), 24 h (PO only).

FIG. 9 show Plasma concentration across time following administration of compound 69 as a single bolus via IV and PO gavage to mice. n=3/timepoint. Timepoints include 5 min (IV only), 15 min, 30 min, 1 h, 2 h, 4 h, 6 h, 8 h, 10 h, 16 h (PO only), 24 h (PO only).

FIG. 10 shows Plasma concentration across time following administration of compound 54 as a single bolus via IV and PO gavage to mice. n=4/timepoint for IV, n=6/timepoint PO. Timepoints include 5 min (IV only), 15 min, 30 min, 1 h, 2 h, 4 h, 8 h, 16 h, 24 h (PO only).

### DETAILED DESCRIPTION

The present disclosure provides compounds and methods for inhibiting DNA-dependent protein kinase (DNA-PK). Aspects of the present disclosure also include methods of using the compounds to treat diseases, including, but not limited to, cancer. In certain embodiments, the compounds inhibit DNA-PK and thus sensitize cancers to therapies such as chemotherapy and radiotherapy. Certain compounds of the present disclosure are in the form of prodrugs that release the DNA-PK inhibitor in hypoxic tissue such as is known to occur in cancers. Aspects of the present disclosure also include methods of using the compounds for repairing a DNA break in a target genomic region or for modifying expression of one or more genes or proteins.

### Compounds

### Formula (II)

Aspects of the present disclosure include compounds of formula (II):

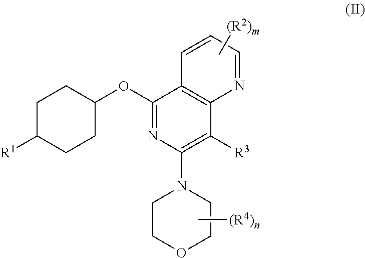

wherein:
R¹ is selected from 5- to 10-membered heteroaryl, NR⁶R⁷, C(O)R⁷, C(O)NR⁶R⁷, N═C(NR⁶R⁷)₂, wherein each heteroaryl is optionally substituted with from 1 to 5 R⁸ substituents;
each R² is independently selected from C₂-C₆-alkynyl, halo, OR⁵, NR⁶R⁷, COOR⁶, C(O)NR⁶R⁷, C₃-C₈-cycloalkyl, 3- to 8-membered heterocycloalkyl, 5- to 10-membered aryl, 5- to 10-membered heteroaryl, S—C₁-C₆-alkyl, S(O)₂—C₁-C₆-alkyl, and S(O)₂—NR¹⁰R¹⁰, wherein each alkyl, alkynyl, cycloalkyl, heterocycloalkyl, aryl, heteroaryl is optionally substituted with from 1 to 5 R⁹ substituents;
R³ is selected from H, halo, C₁-C₆-alkyl and C₁-C₆-haloalkyl;
each R⁴ is independently selected from halo, C₁-C₆-alkyl and C₁-C₆-haloalkyl, wherein two R⁴ groups are optionally linked to form a 5- to 7-membered heterocycloalkyl;
each R⁵ is independently selected from H and C₁-C₆-alkyl, wherein each alkyl is optionally substituted with from 1 to 5 R⁹ substituents;
each R⁶ is independently selected from H, C₁-C₆-alkyl and COOR⁵;
each R⁷ is independently selected from H, OR⁵, cyano, C₁-C₆-alkyl, C₃-C₈-cycloalkyl, 3- to 10-membered heterocycloalkyl, 5- to 10-membered aryl, 5- to 10-membered heteroaryl, C(O)—C₁-C₆-alkyl, C(O)—(C₃-C₈-cycloalkyl), C(O)-(3- to 8-membered heterocycloalkyl), C(O)-(5- to 10-membered aryl), C(O)-(5- to 10-membered heteroaryl), C(O)—O—C₁-C₆-alkyl, S(O)₂—C₁-C₆-alkyl, S(O)₂—(C₃-C₈-cycloalkyl), S(O)₂-(3- to 8-membered heterocycloalkyl), S(O)₂-(5- to 10-membered aryl), S(O)₂-(5- to 10-membered heteroaryl), C(O)NR¹⁰R¹⁰, C(═NH)NH₂, and S(O)₂—NR¹⁰R¹⁰, wherein each alkyl, cycloalkyl, heterocycloalkyl, aryl and heteroaryl is optionally substituted with from 1 to 5 R¹¹ substituents;
each R⁸ is independently selected from C₁-C₆-alkyl, C₁-C₆-haloalkyl, and halo; each R⁹ is independently selected from OH, oxo, C₁-C₆-alkyl, 3- to 8-membered heterocycloalkyl, and 5- to 10-membered aryl, wherein each alkyl, heterocycloalkyl and aryl is optionally substituted with from 1 to 5 R¹¹ substituents;
each R¹⁰ is independently selected from H, C₁-C₆-alkyl, C₁-C₆-alkoxy, 5- to 10-membered aryl, and S(O)₂—C₁-C₆-alkyl, wherein each alkyl and aryl is optionally substituted with from 1 to 5 R¹¹ substituents;
each R¹¹ is independently selected from OH, oxo, halo, cyano, C₁-C₆-alkyl, C₁-C₆-haloalkyl, C₁-C₆-alkoxy, C₃-C₈-cycloalkyl, 3- to 8-membered heterocycloalkyl, 5- to 10-membered aryl, 5- to 10-membered heteroaryl, NR⁶R⁷, C(O)NR⁶R⁷, C(O)OR⁷, and S(O)₂NR⁶R⁷, wherein each alkyl, haloalkyl, alkoxy, cycloalkyl, heterocycloalkyl, aryl and heteroaryl is optionally substituted with 1 to 5 R¹² substituents;
each R¹² is independently selected from halo, OH, oxo, cyano, C₁-C₆-alkyl, C₁-C₆-haloalkyl, C₁-C₆-alkoxy, 3- to 8-membered heterocycloalkyl, 5- to 10-membered heteroaryl, nitro, and NR¹⁰R¹⁰, wherein each alkyl, haloalkyl, alkoxy, heterocycloalkyl and heteroaryl is optionally substituted with 1 to 5 R¹³ substituents;
each R¹³ is independently selected from OH, halo, C₁-C₆-alkyl, and C₁-C₆-haloalkyl;
m is 0 or an integer selected from 1, 2 and 3; and
n is 0 or an integer selected from 1, 2, 3 and 4,
or a prodrug or a pharmaceutically acceptable salt thereof.

In certain embodiments, R¹ is selected from 5- to 10-membered heteroaryl, NR⁶R⁷, C(O)R⁷, C(O)NR⁶R⁷, N═C(NR⁶R⁷)₂, wherein each heteroaryl is optionally substituted with from 1 to 5 R⁸ substituents. In some instances, R¹ is 5- to 10-membered heteroaryl. For example, in some cases, R¹ is imidazolyl, or pyrazinyl, and the like. In some instances, R¹ is NR⁶R⁷. For example, in some cases, R¹ is NHR⁷. In some instances, R¹ is C(O)R⁷. For example, in some cases, R¹ is C(O)-(3- to 10-membered heterocycloalkyl) (e.g., C(O)-morpholinyl), or C(O)-(5- to 10-membered heteroaryl) (e.g., C(O)-pyrazinyl). In some instances, R¹ is C(O)NR⁶R⁷. For example, in some cases, R¹ is C(O)NHR⁷. In some instances, R¹ is N═C(NR⁶R⁷)₂. For example, in some cases, R¹ is N═C(NCH₃CH₃)₂.

In some instances of R¹, each heteroaryl is optionally substituted with from 1 to 5 R⁸ substituents. In some instances, R¹ is not substituted. In some instances, R¹ is substituted with from 1 to 5 R⁸ substituents. In some instances, R¹ is substituted with one R⁸ substituent. In some instances, R¹ is substituted with two R⁸ substituents. In some instances, R¹ is substituted with three R⁸ substituents. In some instances, R¹ is substituted with four R⁸ substituents. In some instances, R¹ is substituted with five R⁸ substituents.

In certain embodiments, each R² is independently selected from C₂-C₆-alkynyl, halo, OR⁵, NR⁶R⁷, COOR⁶, C(O)NR⁶R⁷, C₃-C₈-cycloalkyl, 3- to 8-membered heterocycloalkyl, 5- to 10-membered aryl, 5- to 10-membered heteroaryl, S—C₁-C₆-alkyl, S(O)₂—C₁-C₆-alkyl, and S(O)₂—NR¹⁰R¹⁰, wherein each alkyl, alkynyl, cycloalkyl, heterocycloalkyl, aryl, heteroaryl is optionally substituted with from 1 to 5 R⁹ substituents. In some instances, R² is C₂-C₆-alkynyl. For example, in some cases, R² is C₂-alkynyl, C₃-alkynyl, or C₄-alkynyl (e.g., but-1-ynyl). In some instances, R² is halo (e.g., F, Cl, Br or I). In some instances, R² is OR⁵. For example, in some cases, R² is OH. In some instances, R² is NR⁶R⁷. For example, in some cases, R² is NH₂ or NHR⁷. For example, in some cases, R² is NHS(O)₂—(C₁-C₆-alkyl) (e.g., NHS(O)₂CH₃). In some instances, R² is COOR⁶. For example, in some cases, R² is COOH or COO—(C₁-C₆-alkyl). In some instances, R² is C(O)NR⁶R⁷. For example, in some cases, R² is C(O)NH₂ or C(O)NHR⁷. In some instances, R² is C₃-C₈-cycloalkyl. In some instances, R² is 3- to 8-membered heterocycloalkyl. In some instances, R² is 5- to 10-membered aryl. For example, in some cases, R² is phenyl. In some instances, R² is 5- to 10-membered heteroaryl. For example, in some cases, R² is pyrazolyl, imidazolyl, isoxazolyl, triazolyl, tetrazolyl, and the like. In some instances, R² is S—C₁-C₆-alkyl. For example, in some cases, R² is S-methyl. In some instances, R² is S(O)₂—C₁-C₆-alkyl. For example, in some cases, R² is S(O)₂-methyl. In some instances, R² is S(O)₂—NR¹⁰R¹⁰. For example, in some cases, R² is S(O)₂NH₂.

In some instances of R², each alkyl, alkynyl, cycloalkyl, heterocycloalkyl, aryl, heteroaryl is optionally substituted with from 1 to 5 R⁹ substituents. In some instances, R² is not substituted. In some instances, R² is substituted with from 1 to 5 R⁹ substituents. In some instances, R² is substituted with one R⁹ substituent. In some instances, R² is substituted with two R⁹ substituents. In some instances, R² is substituted with three R⁹ substituents. In some instances, R² is substituted with four R⁹ substituents. In some instances, R² is substituted with five R⁹ substituents.

In certain embodiments, R³ is selected from H, halo, C₁-C₆-alkyl and C₁-C₆-haloalkyl. In some instances, R³ is H. In some instances, R³ is halo (e.g., F, Cl, Br or I). In some instances, R³ is C₁-C₆-alkyl. In some instances, R³ is C₁-C₆-haloalkyl.

In certain embodiments, each R⁴ is independently selected from halo, C₁-C₆-alkyl and C₁-C₆-haloalkyl, wherein two R⁴ groups are optionally linked to form a 5- to 7-membered heterocycloalkyl. In some instances, R⁴ is halo (e.g., F, Cl, Br or I). In some instances, R⁴ is C₁-C₆-alkyl. For example, in some cases, R⁴ is methyl. In some instances, R⁴ is C₁-C₆-haloalkyl. In some instances, two R⁴ groups are optionally linked to form a 5- to 7-membered heterocycloalkyl. For example, in some cases, n is 2, and thus two R⁴ groups are attached to the morpholine ring in the compound of formula (II). In some instances, the two R⁴ groups attached to the morpholine ring are linked to form a heterocycloalkyl, which includes the nitrogen from the morpholine ring. Accordingly, a compound of formula (II) may include a bridged cyclic structure, where the morpholine ring includes a bridge formed by linking two R⁴ groups. In some cases, two R⁴ groups are linked to form a 5-membered heterocycloalkyl, where the heterocycloalkyl includes the nitrogen from the morpholine ring. In some cases, two R⁴ groups are linked to form a 6-membered heterocycloalkyl, where the heterocycloalkyl includes the nitrogen from the morpholine ring. In some cases, two R⁴ groups are linked to form a 7-membered heterocycloalkyl, where the heterocycloalkyl includes the nitrogen from the morpholine ring.

In certain embodiments, each R⁵ is independently selected from H and C₁-C₆-alkyl, wherein each alkyl is optionally substituted with from 1 to 5 R⁹ substituents. In some instances, R⁵ is H. In some instances, R⁵ is C₁-C₆-alkyl.

In some instances of R⁵, each alkyl is optionally substituted with from 1 to 5 R⁹ substituents. In some instances, R⁵ is not substituted. In some instances, R⁵ is substituted with from 1 to 5 R⁹ substituents. In some instances, R⁵ is substituted with one R⁹ substituent. In some instances, R⁵ is substituted with two R⁹ substituents. In some instances, R⁵ is substituted with three R⁹ substituents. In some instances, R⁵ is substituted with four R⁹ substituents. In some instances, R⁵ is substituted with five R⁹ substituents.

In certain embodiments, each R⁶ is independently selected from H, C₁-C₆-alkyl and COOH. In some instances, R⁶ is H. In some instances, R⁶ is C₁-C₆-alkyl. For example, in some cases, R⁶ is methyl. In some instances, R⁶ is COOR⁵. For example, in some cases, R⁶ is COOH.

In certain embodiments, each R⁷ is independently selected from H, OR⁵, cyano, C₁-C₆-alkyl, C₃-C₈-cycloalkyl, 3- to 10-membered heterocycloalkyl, 5- to 10-membered aryl, 5- to 10-membered heteroaryl, C(O)—C₁-C₆-alkyl, C(O)—(C₃-C₈-cycloalkyl), C(O)-(3- to 8-membered heterocycloalkyl), C(O)-(5- to 10-membered aryl), C(O)-(5- to 10-membered heteroaryl), C(O)—O—C₁-C₆-alkyl, S(O)₂—C₁-C₆-alkyl, S(O)₂—(C₃-C₈-cycloalkyl), S(O)₂-(3- to 8-membered heterocycloalkyl), S(O)₂-(5- to 10-membered aryl), S(O)₂-(5- to 10-membered heteroaryl), C(O)NR¹⁰R¹⁰, C(═NH)NH₂, and S(O)₂—NR¹⁰R¹⁰, wherein each alkyl, cycloalkyl, heterocycloalkyl, aryl and heteroaryl is optionally substituted with from 1 to 5 R¹¹ substituents. In some instances, R⁷ is H. In some instances, R⁷ is OR⁵. For example, in some cases, R⁷ is OH. In some instances, R⁷ is cyano. In some instances, R⁷ is C₁-C₆-alkyl. For example, in some cases, R⁷ is methyl. In some cases, R⁷ is ethyl. In some cases, R⁷ is propyl. In some instances, R⁷ is C₃-C₈-cycloalkyl. In some instances, R⁷ is 3- to 10-membered heterocycloalkyl. For example, in some cases, R⁷ is tetrahydro-2H-pyranyl, 5,6,7,8-tetrahydropyrido[4,3-d]pyrimidinyl, 5,6,7,8-tetrahydro-1,6-naphthyridinyl, 5,6,7,8-tetrahydropyrido[3,4-d]pyrimidinyl, 1,2,3,4-tetrahydro-2,7-naphthyridinyl, or pyrazolo[1,5-a]pyrimidinyl, and the like. In some instances, R⁷ is 5- to 10-membered aryl. In some instances, R⁷ is 5- to 10-membered heteroaryl. For example, in some cases, R⁷ is pyrimidinyl, 5H-pyrrolo[3,2-d]pyrimidinyl, furo[3,2-d]pyrimidinyl, purinyl, pyridinyl, or pyrazinyl, and the like. In some instances, R⁷ is C(O)—C₁-C₆-alkyl. For example, in some cases, R⁷ is C(O)-methyl or C(O)-ethyl. In some instances, R⁷ is C(O)—(C₃-C₈-cycloalkyl). In some instances, R⁷ is C(O)-(3- to 8-membered heterocycloalkyl). In some instances, R⁷ is C(O)-(5- to 10-membered aryl). In some instances, R⁷ is C(O)-(5- to 10-membered heteroaryl). For example, in some cases, R⁷ is C(O)-pyrrolyl, C(O)— isoxazolyl, C(O)-pyridinyl, or C(O)-pyrimidinyl. In some instances, R⁷ is C(O)—O—C₁-C₆-alkyl. In some instances, R⁷ is S(O)₂—C₁-C₆-alkyl. For example, in some cases, R⁷ is S(O)₂-methyl. In some cases, R⁷ is S(O)₂-ethyl. In some cases, R⁷ is S(O)₂-propyl. In some instances, R⁷ is S(O)₂—(C₃-C₈-cycloalkyl). For example, in some cases, R⁷ is S(O)₂-cyclopropyl. In some cases, R⁷ is S(O)₂-cyclobutyl. In some cases, R⁷ is S(O)₂-cyclopentyl. In some instances, R⁷ is S(O)₂-(3- to 8-membered heterocycloalkyl). In some instances, R⁷ is S(O)₂-(5- to 10-membered aryl). In some instances, R⁷ is S(O)₂-(5- to 10-membered heteroaryl). In some instances, R⁷ is C(O)NR¹⁰R¹⁰. For example, in some cases, R⁷ is C(O)NH₂ or C(O)N(CH₃)₂. In some instances, R⁷ is C(═NH)NH₂. In some instances, R⁷ is S(O)₂—NR¹⁰R¹⁰. For example, in some cases, R⁷ is S(O)₂NH₂ or S(O)₂N(CH₃)₂.

In some instances of R⁷, each alkyl, cycloalkyl, heterocycloalkyl, aryl and heteroaryl is optionally substituted with from 1 to 5 R¹¹ substituents. In some instances, R⁷ is not substituted. In some instances, R⁷ is substituted with from 1 to 5 R¹¹ substituents. In some instances, R⁷ is substituted with one R¹¹ substituent. In some instances, R⁷ is substituted with two R¹¹ substituents. In some instances, R⁷ is substituted with three R¹¹ substituents. In some instances, R⁷ is substituted with four R¹¹ substituents. In some instances, R⁷ is substituted with five R¹¹ substituents.

In certain embodiments, each R⁸ is independently selected from C₁-C₆-alkyl, C₁-C₆-haloalkyl, and halo. In some instances, R⁸ is C₁-C₆-alkyl. For example, in some cases, R⁸ is methyl or ethyl. In some instances, R⁸ is C₁-C₆-haloalkyl. In some instances, R⁸ is and halo (e.g., F, Cl, Br or I).

In certain embodiments, each R⁹ is independently selected from OH, oxo, C₁-C₆-alkyl, 3- to 8-membered heterocycloalkyl, and 5- to 10-membered aryl, wherein each alkyl, heterocycloalkyl and aryl is optionally substituted with from 1 to 5 R¹¹ substituents. In some instances, R⁹ is OH. In some instances, R⁹ is oxo. In some instances, R⁹ is C₁-C₆-alkyl. For example, in some cases, R⁹ is methyl. In some instances, R⁹ is 3- to 8-membered heterocycloalkyl. In some instances, R⁹ is 5- to 10-membered aryl.

In some instances of R⁹, each alkyl, heterocycloalkyl and aryl is optionally substituted with from 1 to 5 R¹¹ substituents. In some instances, R⁹ is not substituted. In some instances, R⁹ is substituted with from 1 to 5 R¹¹ substituents. In some instances, R⁹ is substituted with one R¹¹ substituent. In some instances, R⁹ is substituted with two R¹¹ substituents. In some instances, R⁹ is substituted with three R¹¹ substituents. In some instances, R⁹ is substituted with four R¹¹ substituents. In some instances, R⁹ is substituted with five R¹¹ substituents.

In certain embodiments, each R¹⁰ is independently selected from H, C₁-C₆-alkyl, C₁-C₆-alkoxy, 5- to 10-membered aryl, and S(O)₂—C₁-C₆-alkyl, wherein each alkyl and aryl is optionally substituted with from 1 to 5 R¹¹ substituents. In some instances, R¹⁰ is H. In some instances, R¹⁰ is C₁-C₆-alkyl. For example, in some cases, R¹⁰ is methyl. In some instances, R¹⁰ is C₁-C₆-alkoxy. For example, in some cases, R¹⁰ is methoxy. In some instances, R¹⁰ is 5- to 10-membered aryl. In some instances, R¹⁰ is S(O)₂—C₁-C₆-alkyl. For example, in some cases, R¹⁰ is S(O)₂-methyl.

In some instances of R¹⁰, each alkyl and aryl is optionally substituted with from 1 to 5 R¹¹ substituents. In some instances, R¹⁰ is not substituted. In some instances, R¹⁰ is substituted with from 1 to 5 R¹¹ substituents. In some instances, R¹⁰ is substituted with one R¹¹ substituent. In some instances, R¹⁰ is substituted with two R¹¹ substituents. In some instances, R¹⁰ is substituted with three R¹¹ substituents. In some instances, R¹⁰ is substituted with four R¹¹ substituents. In some instances, R¹⁰ is substituted with five R¹¹ substituents.

In certain embodiments, each R¹¹ is independently selected from OH, oxo, halo, cyano, C₁-C₆-alkyl, C₁-C₆-haloalkyl, C₁-C₆-alkoxy, C₃-C₈-cycloalkyl, 3- to 8-membered heterocycloalkyl, 5- to 10-membered aryl, 5- to 10-membered heteroaryl, NR⁶R⁷, C(O)NR⁶R⁷, C(O)OR⁷, and S(O)₂NR⁶R⁷, wherein each alkyl, haloalkyl, alkoxy, cycloalkyl, heterocycloalkyl, aryl and heteroaryl is optionally substituted with 1 to 5 R¹² substituents. In some instances, R¹¹ is OH. In some instances, R¹¹ is oxo. In some instances, R¹¹ is halo (e.g., F, Cl, Br or I). In some instances, R¹¹ is cyano. In some instances, R¹¹ is C₁-C₆-alkyl. For example, in some cases, R¹¹ is methyl, or ethyl. In some instances, R¹¹ is C₁-C₆-haloalkyl. For example, in some cases, R¹¹ is fluoromethyl, difluoromethyl or trifluoromethyl. In some instances, R¹¹ is C₁-C₆-alkoxy. For example, in some cases, R¹¹ is methoxy or ethoxy. In some cases, R¹¹ is heterocycloalkyl-O— (e.g., pyrrolidinyl-O—). In some instances, R¹¹ is C₃-C₈-cycloalkyl. In some instances, R¹¹ is 3- to 8-membered heterocycloalkyl. For example, in some cases, R¹¹ is piperazinyl, 1,2,3,6-tetrahydropyridinyl, piperidinyl, morpholinyl, (1R,5S)-3,8-diazabicyclo[3.2.1]octanyl, octahydropyrrolo[3,4-c]pyrrolyl, 3,6-diazabicyclo[3.1.1]heptanyl, and the like. In some instances, R¹¹ is 5- to 10-membered aryl. In some instances, R¹¹ is 5- to 10-membered heteroaryl. In some instances, R¹¹ is NR⁶R⁷. For example, in some cases, R¹¹ is NH₂ or NHR⁷. In some instances, R¹¹ is C(O)NR⁶R⁷. For example, in some cases, R¹¹ is C(O)NH₂ or C(O)NHR⁷. In some instances, R¹¹ is C(O)OR⁷. For example, in some cases, R¹¹ is COOH. In some instances, R¹¹ is S(O)₂NR⁶R⁷. For example, in some cases, R¹¹ is S(O)₂NH₂ or S(O)₂NHR⁷.

In some instances of R¹¹, each alkyl, haloalkyl, alkoxy, cycloalkyl, heterocycloalkyl, aryl and heteroaryl is optionally substituted with from 1 to 5 R¹² substituents. In some instances, R¹¹ is not substituted. In some instances, R¹¹ is substituted with from 1 to 5 R¹² substituents. In some instances, R¹¹ is substituted with one R¹² substituent. In some instances, R¹¹ is substituted with two R¹² substituents. In some instances, R¹¹ is substituted with three R¹² substituents. In some instances, R¹¹ is substituted with four R¹² substituents. In some instances, R¹¹ is substituted with five R¹² substituents.

In certain embodiments, each R¹² is independently selected from halo, OH, oxo, cyano, C₁-C₆-alkyl, C₁-C₆-haloalkyl, C₁-C₆-alkoxy, 3- to 8-membered heterocycloalkyl, 5- to 10-membered heteroaryl, nitro, and NR¹⁰R¹⁰, wherein each alkyl, haloalkyl, alkoxy, heterocycloalkyl and heteroaryl is optionally substituted with 1 to 5 R¹³ substituents. In some instances, R¹² is halo (e.g., F, Cl, Br or I). In some instances, R¹² is OH. In some instances, R¹² is oxo. In some instances, R¹² is cyano. In some instances, R¹² is C₁-C₆-alkyl. For example, in some cases, R¹² is methyl or ethyl. In some instances, R¹² is C₁-C₆-haloalkyl. In some instances, R¹² is C₁-C₆-alkoxy. In some instances, R¹² is 3- to 8-membered heterocycloalkyl. For example, in some cases, R¹² is piperazinyl, morpholinyl, oxetanyl, and the like. In some instances, R¹² is 5- to 10-membered heteroaryl. In some instances, R¹² is nitro. In some instances, R¹² is NR¹⁰R¹⁰. For example, in some cases, R¹² is NH₂ or NHR¹⁰. In some instances of NR¹⁰R¹⁰, both R¹⁰ groups are the same (e.g., both R¹⁰ groups are H or CH₃). In some instances of NR¹⁰R¹⁰, the R¹⁰ groups are different (e.g., NHR¹⁰, such as NHCH₃).

In some instances of R¹², each alkyl, haloalkyl, alkoxy, heterocycloalkyl and heteroaryl is optionally substituted with from 1 to 5 R¹³ substituents. In some instances, R¹² is not substituted. In some instances, R¹² is substituted with from 1 to 5 R¹³ substituents. In some instances, R¹² is substituted with one R¹³ substituent. In some instances, R¹² is substituted with two R¹³ substituents. In some instances, R¹² is substituted with three R¹³ substituents. In some instances, R¹² is substituted with four R¹³ substituents. In some instances, R¹² is substituted with five R¹³ substituents.

In certain embodiments, each R¹³ is independently selected from OH, halo, C₁-C₆-alkyl, and C₁-C₆-haloalkyl. In some instances, R¹³ is OH. In some instances, R¹³ is halo (e.g., F, Cl, Br or I). In some instances, R¹³ is C₁-C₆-alkyl. For example, in some cases, R¹³ is methyl. In some instances, R¹³ is C₁-C₆-haloalkyl. For example, in some cases, R¹³ is fluoromethyl, difluoromethyl or trifluoromethyl.

In certain embodiments, m is 0 or an integer selected from 1, 2 and 3. In some instances, m is 0. When m is 0, then R² is not present. In some instances, m is 1. In some instances, m is 2. In some instances, m is 3.

In certain embodiments, n is 0 or an integer selected from 1, 2, 3 and 4. In some instances, n is 0. When n is 0, then R⁴ is not present. In some instances, n is 1. In some instances, n is 2. In some instances, n is 3. In some instances, n is 4.

In certain embodiments of formula (II), the compound includes the following features:
R¹ is NR⁶R⁷;
R⁶ is H;
R⁷ is 5- to 10-membered heteroaryl, wherein heteroaryl is substituted with C₁-C₆-alkyl, wherein alkyl is substituted with oxo and NR²¹R²², wherein each of R²¹ and R²² is independently selected from H and C₁-C₆-alkyl.

In some instances, R¹ is NR⁶R⁷ and R⁶ is H. As such, R¹ is NHR⁷.

In some instances, R⁷ is 5- to 10-membered heteroaryl, wherein heteroaryl is substituted with C₁-C₆-alkyl, wherein alkyl is substituted with oxo and NR²¹R²², wherein each or R²¹ and R²² is independently selected from H and C₁-C₆-alkyl. In some instances, R⁷ is 5- to 10-membered heteroaryl. For example, in some cases, R⁷ is pyrimidinyl, 5H-pyrrolo[3,2-d]pyrimidinyl, furo[3,2-d]pyrimidinyl, or purinyl. In some instances, the heteroaryl is substituted with C₁-C₆-alkyl, wherein alkyl is substituted with oxo and NR²¹R²². In some instances, each of R²¹ and R²² is independently selected from H and C₁-C₆-alkyl. In some instances, R²¹ is H. In some instances, R²¹ is C₁-C₆-alkyl. For example, in some cases, R²¹ is methyl. In some instances, R²² is H. In some instances, R²² is C₁-C₆-alkyl. For example, in some cases, R²² is methyl.

In certain embodiments of formula (II), the compound includes the following features:
R¹ is NR⁶R⁷;
R⁶ is H;
R⁷ is 5- to 10-membered heteroaryl, wherein heteroaryl is substituted with from 2 to 3 R¹¹ substituents;
wherein one R¹¹ substituent is oxo, and each additional R¹¹ substituent is independently selected from OH, halo, cyano, C₁-C₆-alkyl, C₁-C₆-haloalkyl, C₁-C₆-alkoxy, C₃-C₈-cycloalkyl, 3- to 8-membered heterocycloalkyl, 5- to 10-membered aryl, 5- to 10-membered heteroaryl, NR²³R²⁴, C(O)NR²³R²⁴, C(O)OR²⁴, and S(O)₂NR²³R²⁴, wherein each alkyl, haloalkyl, alkoxy, cycloalkyl, heterocycloalkyl, aryl and heteroaryl is optionally substituted with 1 to 5 R¹² substituents;
each of R²³ and R²⁴ is independently selected from H and C₁-C₆-alkyl.

In some instances, R¹ is NR⁶R⁷ and R⁶ is H. As such, R¹ is NHR⁷.

In some instances, R⁷ is 5- to 10-membered heteroaryl, wherein heteroaryl is substituted with from 2 to 3 R¹¹ substituents. In some instances, R⁷ is 5- to 10-membered heteroaryl. For example, in some cases, R⁷ is pyrimidinyl, 5H-pyrrolo[3,2-d]pyrimidinyl, furo[3,2-d]pyrimidinyl, or purinyl. In some instances, the heteroaryl is substituted with from 2 to 3 R¹¹ substituents. In some instances, one R¹¹ substituent is oxo. In some instances, each additional R¹¹ substituent is independently selected from OH, halo, cyano, C₁-C₆-alkyl, C₁-C₆-haloalkyl, C₁-C₆-alkoxy, C₃-C₈-cycloalkyl, 3- to 8-membered heterocycloalkyl, 5- to 10-membered aryl, 5- to 10-membered heteroaryl, NR²³R²⁴, C(O)NR²³R²⁴, C(O)OR²⁴, and S(O)₂NR²³R²⁴. In some instances, R¹¹ is OH. In some instances, R¹¹ is halo (e.g., F, Cl, Br or I). In some instances, R¹¹ is cyano. In some instances, R¹¹ is C₁-C₆-alkyl. For example, in some cases, R¹¹ is methyl. In some instances, R¹¹ is C₁-C₆-haloalkyl. In some instances, R¹¹ is C₁-C₆-alkoxy. In some instances, R¹¹ is C₃-C₈-cycloalkyl. In some instances, R¹¹ is 3- to 8-membered heterocycloalkyl. In some instances, R¹¹ is 5- to 10-membered aryl. In some instances, R¹¹ is 5- to 10-membered heteroaryl. In some instances, R¹¹ is NR²³R²⁴. For example, in some cases, R¹¹ is NH₂, NHCH₃ or N(CH₃)₂. In some instances, R¹¹ is C(O)NR²³R²⁴. For example, in some cases, R¹¹ is C(O)NH₂, C(O)NHCH₃ or C(O)N(CH₃)₂. In some instances, R¹¹ is C(O)OR²⁴. For example, in some cases, R¹¹ is COOH. In some instances, R¹¹ is S(O)₂NR²³R²⁴. For example, in some cases, R¹¹ is S(O)₂NH₂ or S(O)₂N(CH₃)₂.

In some instances of R¹¹, each alkyl, haloalkyl, alkoxy, cycloalkyl, heterocycloalkyl, aryl and heteroaryl is optionally substituted with from 1 to 5 R¹² substituents. In some instances, R¹¹ is not substituted. In some instances, R¹¹ is substituted with from 1 to 5 R¹² substituents. In some instances, R¹¹ is substituted with one R¹² substituent. In some instances, R¹¹ is substituted with two R¹² substituents. In some instances, R¹¹ is substituted with three R¹² substituents. In some instances, R¹¹ is substituted with four R¹² substituents. In some instances, R¹¹ is substituted with five R¹² substituents.

In some instances, each of R²³ and R²⁴ is independently selected from H and C₁-C₆-alkyl. In some instances, R²³ is H. In some instances, R²³ is C₁-C₆-alkyl. For example, in some cases, R²³ is methyl. In some instances, R²⁴ is H. In some instances, R²⁴ is C₁-C₆-alkyl. For example, in some cases, R²⁴ is methyl.

### Formula (IIa)

In certain embodiments of the compound of formula (II), the compound is a compound of formula (IIa):

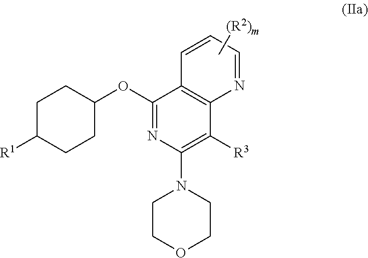

wherein:
R¹ is NR⁶R⁷;
R⁶ is H;
R⁷ is selected from C(O)—C₁-C₆-alkyl, C(O)—(C₃-C₈-cycloalkyl), C(O)-(3- to 8-membered heterocycloalkyl), C(O)-(5- to 10-membered aryl), C(O)-(5- to 10-membered heteroaryl), C(O)NR¹⁰R¹⁰, and C(═NH)NH₂, wherein each alkyl, cycloalkyl, heterocycloalkyl, aryl and heteroaryl is optionally substituted with from 1 to 5 R¹¹ substituents.

In certain embodiments of formula (IIa), R², R³ and m are as described herein in relation to compounds of formula (II).

In certain embodiments, R¹ is NR⁶R⁷.

In certain embodiments, R⁶ is H.

In certain embodiments, R⁷ is selected from C(O)—C₁-C₆-alkyl, C(O)—(C₃-C₈-cycloalkyl), C(O)-(3- to 8-membered heterocycloalkyl), C(O)-(5- to 10-membered aryl), C(O)-(5- to 10-membered heteroaryl), C(O)NR¹⁰R¹⁰, and C(═NH)NH₂, wherein each alkyl, cycloalkyl, heterocycloalkyl, aryl and heteroaryl is optionally substituted with from 1 to 5 R¹¹ substituents. In some instances, R⁷ is C(O)—C₁-C₆-alkyl. For example, in some cases, R⁷ is C(O)-methyl or C(O)-ethyl. In some instances, R⁷ is C(O)—(C₃-C₈-cycloalkyl). In some instances, R⁷ is C(O)-(3- to 8-membered heterocycloalkyl). In some instances, R⁷ is C(O)-(5- to 10-membered aryl). In some instances, R⁷ is C(O)-(5- to 10-membered heteroaryl). For example, in some cases, R⁷ is C(O)-pyrrolyl, C(O)— isoxazolyl, C(O)-pyridinyl, or C(O)-pyrimidinyl. In some instances, R⁷ is C(O)—O—C₁-C₆-alkyl. In some instances, R⁷ is C(O)NR¹⁰R¹⁰. For example, in some cases, R⁷ is C(O)NH₂ or C(O)N(CH₃)₂. In some instances, R⁷ is C(═NH)NH₂.

In some instances of R⁷, each alkyl, cycloalkyl, heterocycloalkyl, aryl and heteroaryl is optionally substituted with from 1 to 5 R¹¹ substituents. In some instances, R⁷ is not substituted. In some instances, R⁷ is substituted with from 1 to 5 R¹¹ substituents. In some instances, R⁷ is substituted with one R¹¹ substituent. In some instances, R⁷ is substituted with two R¹¹ substituents. In some instances, R⁷ is substituted with three R¹¹ substituents. In some instances, R⁷ is substituted with four R¹¹ substituents. In some instances, R⁷ is substituted with five R¹¹ substituents.

### Formula (IIb)

In certain embodiments of the compound of formula (II), the compound is a compound of formula (IIb):

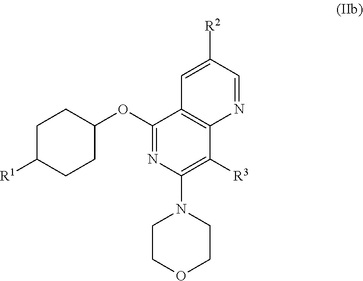

wherein:
R² is NR⁶R⁷;
R⁶ is H;
R⁷ is selected from C(O)NR¹⁰R¹⁰, S(O)₂—C₁-C₆-alkyl, S(O)₂—(C₃-C₈-cycloalkyl), S(O)₂-(3- to 8-membered heterocycloalkyl), S(O)₂-(5- to 10-membered aryl), S(O)₂-(5- to 10-membered heteroaryl), and S(O)₂—NR¹⁰R¹⁰, wherein each alkyl, cycloalkyl, heterocycloalkyl, aryl and heteroaryl is optionally substituted with from 1 to 5 R¹¹ substituents.

In certain embodiments of formula (IIb), R¹ and R³ are as described herein in relation to compounds of formula (II).

In certain embodiments, R² is NR⁶R⁷.

In certain embodiments, R⁶ is H.

In certain embodiments, R⁷ is selected from C(O)NR¹⁰R¹⁰, S(O)₂—C₁-C₆-alkyl, S(O)₂—(C₃-C₈-cycloalkyl), S(O)₂-(3- to 8-membered heterocycloalkyl), S(O)₂-(5- to 10-membered aryl), S(O)₂-(5- to 10-membered heteroaryl), and S(O)₂—NR¹⁰R¹⁰, wherein each alkyl, cycloalkyl, heterocycloalkyl, aryl and heteroaryl is optionally substituted with from 1 to 5 R¹¹ substituents. In some instances, R⁷ is C(O)NR¹⁰R¹⁰. For example, in some cases, R⁷ is C(O)NH₂ or C(O)N(CH₃)₂. In some instances, R⁷ is S(O)₂—C₁-C₆-alkyl. For example, in some cases, R⁷ is S(O)₂-methyl. In some cases, R⁷ is S(O)₂-ethyl. In some cases, R⁷ is S(O)₂-propyl. In some instances, R⁷ is S(O)₂—(C₃-C₈-cycloalkyl). For example, in some cases, R⁷ is S(O)₂-cyclopropyl. In some cases, R⁷ is S(O)₂-cyclobutyl. In some cases, R⁷ is S(O)₂-cyclopentyl. In some instances, R⁷ is S(O)₂-(3- to 8-membered heterocycloalkyl). In some instances, R⁷ is S(O)₂-(5- to 10-membered aryl). In some instances, R⁷ is S(O)₂-(5- to 10-membered heteroaryl). In some instances, R⁷ is S(O)₂—NR¹⁰R¹⁰. For example, in some cases, R⁷ is S(O)₂NH₂ or S(O)₂N(CH₃)₂.

In some instances of R⁷, each alkyl, cycloalkyl, heterocycloalkyl, aryl and heteroaryl is optionally substituted with from 1 to 5 R¹¹ substituents. In some instances, R⁷ is not substituted. In some instances, R⁷ is substituted with from 1 to 5 R¹¹ substituents. In some instances, R⁷ is substituted with one R¹¹ substituent. In some instances, R⁷ is substituted with two R¹¹ substituents. In some instances, R⁷ is substituted with three R¹¹ substituents. In some instances, R⁷ is substituted with four R¹¹ substituents. In some instances, R⁷ is substituted with five R¹¹ substituents.

### Formula (IIc)

In certain embodiments of the compound of formula (II), the compound is a compound of formula (IIc):

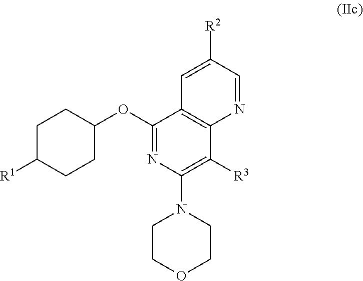

wherein:
R² is OR⁵;
R⁵ is C₁-C₆-alkyl substituted with from 1 to 5 R⁹ substituents.

In certain embodiments of formula (IIc), R¹ and R³ are as described herein in relation to compounds of formula (II).

In certain embodiments, R² is OR⁵.

In certain embodiments, R⁵ is C₁-C₆-alkyl substituted with from 1 to 5 R⁹ substituents. In some instances, R⁵ is substituted with one R⁹ substituent. In some instances, R⁵ is substituted with two R⁹ substituents. In some instances, R⁵ is substituted with three R⁹ substituents. In some instances, R⁵ is substituted with four R⁹ substituents. In some instances, R⁵ is substituted with five R⁹ substituents.

In some instances, R⁵ is methyl substituted with from 1 to 5 R⁹ substituents. In some instances, R⁵ is ethyl substituted with from 1 to 5 R⁹ substituents.

In certain embodiments, the R⁹ substituent on R⁵ is selected from OH, oxo, C₁-C₆-alkyl, 3- to 8-membered heterocycloalkyl, and 5- to 10-membered aryl. In some instances, the R⁹ substituent on R⁵ is OH. In some instances, the R⁹ substituent on R⁵ is 3- to 8-membered heterocycloalkyl. For example, in some cases, the R⁹ substituent on R⁵ is morpholinyl.

### Formula (IId)

In certain embodiments of the compound of formula (II), the compound is a compound of formula (IId):

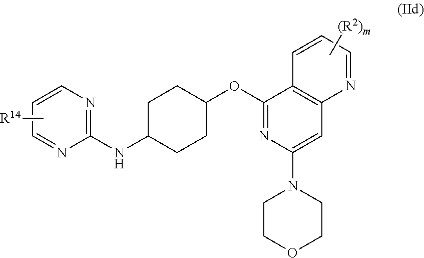

wherein:
R¹⁴ is C₁-C₆-alkoxy, wherein the alkoxy is substituted with 1 to 2 R¹¹ substituents,
wherein when the alkoxy of R¹⁴ is substituted with one R¹¹ substituent, R¹¹ is selected from 3- to 8-membered heterocycloalkyl, 5- to 10-membered heteroaryl, and NR¹⁷R¹⁷, wherein each heterocycloalkyl and heteroaryl is optionally substituted with 1 to 2 R¹⁶ substituents;
wherein when the alkoxy of R¹⁴ is substituted with two R¹⁵ substituents, each R¹⁵ is independently selected from OH, oxo, 3- to 8-membered heterocycloalkyl, and NR¹⁷R¹⁷, wherein heterocycloalkyl is optionally substituted with 1 to 2 R¹⁶ substituents;
each R¹⁶ is independently selected from OH, halo, and C₁-C₆-alkyl;
each R¹⁷ is independently selected from H, C₁-C₆-alkyl, C₁-C₆-alkoxy, 5- to 10-membered aryl, and S(O)₂—C₁-C₆-alkyl, wherein each alkyl and aryl is optionally substituted with from 1 to 2 R¹⁸ substituents;
each R¹⁸ is independently selected from OH, oxo, halo, cyano, C₁-C₆-alkyl, C₁-C₆-haloalkyl, C₁-C₆-alkoxy, C₃-C₈-cycloalkyl, 3- to 8-membered heterocycloalkyl, 5- to 10-membered aryl, 5- to 10-membered heteroaryl, NR¹⁹R²⁰, C(O)NR¹⁹R²⁰, C(O)OR²⁰, and S(O)₂NR¹⁹R²⁰; and
each of R¹⁹ and R²⁰ is independently selected from H and C₁-C₆-alkyl.

In certain embodiments of formula (IId), R² and m are as described herein in relation to compounds of formula (II).

In certain embodiments, R¹⁴ is C₁-C₆-alkoxy, wherein the alkoxy is substituted with 1 to 2 R¹⁵ substituents.

In some instances, R¹⁴ is C₁-C₆-alkoxy substituted with one R¹⁵ substituent. In these embodiments, when the alkoxy of R¹⁴ is substituted with one R¹⁵ substituent, R¹⁵ is selected from 3- to 8-membered heterocycloalkyl, 5- to 10-membered heteroaryl, and NR¹⁷R¹⁷, wherein each heterocycloalkyl and heteroaryl is optionally substituted with 1 to 2 R¹⁶ substituents. In some instances, R¹⁵ is 3- to 8-membered heterocycloalkyl. For example, in some cases, R¹⁵ is azetidinyl, pyrrolidinyl, piperidinyl, piperazinyl, morpholinyl, tetrahydro-2H-pyranyl or thiomorpholinyl. In some instances, R¹⁵ is 5- to 10-membered heteroaryl. For example, in some cases, R¹⁵ is imidazolyl or oxazolyl. In some instances, R¹⁵ is NR¹⁷R¹⁷. For example, in some cases, R¹⁵ is NH₂, NHR¹⁷ or N(CH₃)₂.

In some instances of R¹⁵, each heterocycloalkyl and heteroaryl is optionally substituted with from 1 to 2 R¹⁶ substituents. In some instances, R¹⁵ is not substituted. In some instances, R¹⁵ is substituted with from 1 to 2 R¹⁶ substituents. In some instances, R¹⁵ is substituted with one R¹⁶ substituent. In some instances, R¹⁵ is substituted with two R¹⁶ substituents.

In some instances, R¹⁴ is C₁-C₆-alkoxy substituted with two R¹⁵ substituents. In these embodiments, when the alkoxy of R¹⁴ is substituted with two R¹⁵ substituents, each R¹⁵ is independently selected from OH, oxo, 3- to 8-membered heterocycloalkyl, and NR¹⁷R¹⁷, wherein heterocycloalkyl is optionally substituted with 1 to 2 R¹⁶ substituents. In some instances, R¹⁵ is OH. In some instances, R¹⁵ is oxo. In some instances, R¹⁵ is 3- to 8-membered heterocycloalkyl. For example, in some cases, R¹⁵ is azetidinyl, pyrrolidinyl, piperidinyl, piperazinyl, morpholinyl, tetrahydro-2H-pyranyl or thiomorpholinyl. In some instances, R¹⁵ is NR¹⁷R¹⁷. For example, in some cases, R¹⁵ is NH₂, NHR¹⁷ or N(CH₃)₂.

In some instances of R¹⁵, each heterocycloalkyl is optionally substituted with from 1 to 2 R¹⁶ substituents. In some instances, R¹⁵ is not substituted. In some instances, R¹⁵ is substituted with from 1 to 2 R¹⁶ substituents. In some instances, R¹⁵ is substituted with one R¹⁶ substituent. In some instances, R¹⁵ is substituted with two R¹⁶ substituents.

In certain embodiments, each R¹⁶ is independently selected from OH, halo, and C₁-C₆-alkyl. In some instances, R¹⁶ is OH. In some instances, R¹⁶ is halo (F, Cl, Br or I). In some instances, R¹⁶ is C₁-C₆-alkyl. For example, in some cases, R¹⁶ is methyl.

In certain embodiments, each R¹⁷ is independently selected from H, C₁-C₆-alkyl, C₁-C₆-alkoxy, 5- to 10-membered aryl, and S(O)₂—C₁-C₆-alkyl, wherein each alkyl and aryl is optionally substituted with from 1 to 2 R¹⁸ substituents. In some instances, R¹⁷ is H. In some instances, R¹⁷ is C₁-C₆-alkyl. For example, in some cases, R¹⁷ is methyl or ethyl. In some instances, R¹⁷ is C₁-C₆-alkoxy. For example, in some cases, R¹⁷ is methoxy. In some instances, R¹⁷ is 5- to 10-membered aryl. In some instances, R¹⁷ is S(O)₂—C₁-C₆-alkyl. For example, in some cases, R¹⁷ is S(O)₂-methyl.

In some instances of R¹⁷, each alkyl and aryl is optionally substituted with from 1 to 2 R¹⁸ substituents. In some instances, R¹⁷ is not substituted. In some instances, R¹⁷ is substituted with from 1 to 2 R¹⁸ substituents. In some instances, R¹⁷ is substituted with one R¹⁸ substituent. In some instances, R¹⁷ is substituted with two R¹⁸ substituents.

In certain embodiments, each R¹⁸ is independently selected from OH, oxo, halo, cyano, C₁-C₆-alkyl, C₁-C₆-haloalkyl, C₁-C₆-alkoxy, C₃-C₈-cycloalkyl, 3- to 8-membered heterocycloalkyl, 5- to 10-membered aryl, 5- to 10-membered heteroaryl, NR¹⁹R²⁰, C(O)NR¹⁹R²⁰, C(O)OR²⁰, and S(O)₂NR¹⁹R²⁰. In some instances, R¹⁸ is OH. In some instances, R¹⁸ is oxo. In some instances, R¹⁸ is halo (e.g., F, Cl, Br or I). In some instances, R¹⁸ is cyano. In some instances, R¹⁸ is C₁-C₆-alkyl. For example, in some cases, R¹⁸ is methyl. In some instances, R¹⁸ is C₁-C₆-haloalkyl. In some instances, R¹⁸ is C₁-C₆-alkoxy. For example, in some cases, R¹⁸ is methoxy. In some instances, R¹⁸ is C₃-C₈-cycloalkyl. In some instances, R¹⁸ is 3- to 8-membered heterocycloalkyl. In some instances, R¹⁸ is 5- to 10-membered aryl. In some instances, R¹⁸ is 5- to 10-membered heteroaryl. In some instances, R¹⁸ is NR¹⁹R²⁰. For example, in some cases, R¹⁸ is NH₂, NHCH₃ or N(CH₃)₂. In some instances, R¹⁸ is C(O)NR¹⁹R²⁰. For example, in some cases, R¹⁸ is C(O)NH₂, C(O)NHCH₃ or C(O)N(CH₃)₂. In some instances, R¹⁸ is C(O)OR²⁰. For example, in some cases, R¹⁸ is COOH. In some instances, R¹⁸ is S(O)₂NR¹⁹R²⁰. For example, in some cases, R¹⁸ is S(O)₂NH₂ or S(O)₂N(CH₃)₂.

In certain embodiments, each of R¹⁹ and R²⁰ is independently selected from H and C₁-C₆-alkyl. In some instances, R¹⁹ is H. In some instances, R¹⁹ is C₁-C₆-alkyl. For example, in some cases, R¹⁹ is methyl. In some instances, R²⁰ is H. In some instances, R²⁰ is C₁-C₆-alkyl. For example, in some cases, R²⁰ is methyl.

### Formula (IIe)

In certain embodiments of the compound of formula (II), the compound is a compound of formula (IIe):

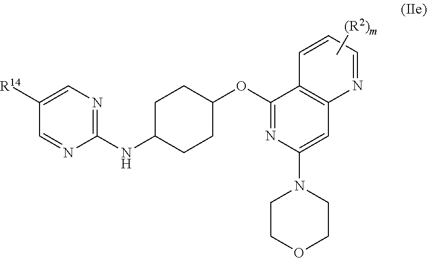

wherein:
R¹⁴ is C₁-C₆-alkoxy, wherein the alkoxy is substituted with 1 to 2 R¹¹ substituents,
wherein when the alkoxy of R¹⁴ is substituted with one R¹¹ substituent, R¹¹ is selected from 3- to 8-membered heterocycloalkyl, 5- to 10-membered heteroaryl, and NR¹⁷R¹⁷, wherein each heterocycloalkyl and heteroaryl is optionally substituted with 1 to 2 R¹⁶ substituents;
wherein when the alkoxy of R¹⁴ is substituted with two R¹⁵ substituents, each R¹⁵ is independently selected from OH, oxo, 3- to 8-membered heterocycloalkyl, and NR¹⁷R¹⁷, wherein heterocycloalkyl is optionally substituted with 1 to 2 R¹⁶ substituents;
each R¹⁶ is independently selected from OH, halo, and C₁-C₆-alkyl;
each R¹⁷ is independently selected from H, C₁-C₆-alkyl, C₁-C₆-alkoxy, 5- to 10-membered aryl, and S(O)₂—C₁-C₆-alkyl, wherein each alkyl and aryl is optionally substituted with from 1 to 2 R¹⁸ substituents;
each R¹⁸ is independently selected from OH, oxo, halo, cyano, C₁-C₆-alkyl, C₁-C₆-haloalkyl, C₁-C₆-alkoxy, C₃-C₈-cycloalkyl, 3- to 8-membered heterocycloalkyl, 5- to 10-membered aryl, 5- to 10-membered heteroaryl, NR¹⁹R²⁰, C(O)NR¹⁹R²⁰, C(O)OR²⁰, and S(O)₂NR¹⁹R²⁰; and
each of R¹⁹ and R²⁰ is independently selected from H and C₁-C₆-alkyl.

In certain embodiments of formula (IIe), R² and m are as described herein in relation to compounds of formula (II). In some instances, m is 0.

In certain embodiments, R¹⁴ is C₁-C₆-alkoxy, wherein the alkoxy is substituted with 1 to 2 R¹⁵ substituents.

In some instances, R¹⁴ is C₁-C₆-alkoxy substituted with one R¹⁵ substituent. In these embodiments, when the alkoxy of R¹⁴ is substituted with one R¹⁵ substituent, R¹⁵ is selected from 3- to 8-membered heterocycloalkyl, 5- to 10-membered heteroaryl, and NR¹⁷R¹⁷, wherein each heterocycloalkyl and heteroaryl is optionally substituted with 1 to 2 R¹⁶ substituents. In some instances, R¹⁵ is 3- to 8-membered heterocycloalkyl. For example, in some cases, R¹⁵ is azetidinyl, pyrrolidinyl, piperidinyl, piperazinyl, morpholinyl, tetrahydro-2H-pyranyl or thiomorpholinyl. In some instances, R¹⁵ is 5- to 10-membered heteroaryl. For example, in some cases, R¹⁵ is imidazolyl or oxazolyl. In some instances, R¹⁵ is NR¹⁷R¹⁷. For example, in some cases, R¹⁵ is NH₂, NHR¹⁷, N(CH₃)₂, or N(CH₃)(OCH₃).

In some instances of R¹⁵, each heterocycloalkyl and heteroaryl is optionally substituted with from 1 to 2 R¹⁶ substituents. In some instances, R¹⁵ is not substituted. In some instances, R¹⁵ is substituted with from 1 to 2 R¹⁶ substituents. In some instances, R¹⁵ is substituted with one R¹⁶ substituent. In some instances, R¹⁵ is substituted with two R¹⁶ substituents.

In some instances, R¹⁴ is C₁-C₆-alkoxy substituted with two R¹⁵ substituents. In these embodiments, when the alkoxy of R¹⁴ is substituted with two R¹⁵ substituents, each R¹⁵ is independently selected from OH, oxo, 3- to 8-membered heterocycloalkyl, and NR¹⁷R¹⁷, wherein heterocycloalkyl is optionally substituted with 1 to 2 R¹⁶ substituents. In some instances, R¹⁵ is OH. In some instances, R¹⁵ is oxo. In some instances, R¹⁵ is 3- to 8-membered heterocycloalkyl. For example, in some cases, R¹⁵ is azetidinyl, pyrrolidinyl, piperidinyl, piperazinyl, morpholinyl, tetrahydro-2H-pyranyl or thiomorpholinyl. In some instances, R¹⁵ is NR¹⁷R¹⁷. For example, in some cases, R¹⁵ is NH₂, NHR¹⁷, N(CH₃)₂, or N(CH₃)(OCH₃).

In some instances of R¹⁵, each heterocycloalkyl is optionally substituted with from 1 to 2 R¹⁶ substituents. In some instances, R¹⁵ is not substituted. In some instances, R¹⁵ is substituted with from 1 to 2 R¹⁶ substituents. In some instances, R¹⁵ is substituted with one R¹⁶ substituent. In some instances, R¹⁵ is substituted with two R¹⁶ substituents.

In certain embodiments, each R¹⁶ is independently selected from OH, halo, and C₁-C₆-alkyl. In some instances, R¹⁶ is OH. In some instances, R¹⁶ is halo (F, Cl, Br or I). In some instances, R¹⁶ is C₁-C₆-alkyl. For example, in some cases, R¹⁶ is methyl.

In certain embodiments, each R¹⁷ is independently selected from H, C₁-C₆-alkyl, C₁-C₆-alkoxy, 5- to 10-membered aryl, and S(O)₂—C₁-C₆-alkyl, wherein each alkyl and aryl is optionally substituted with from 1 to 2 R¹⁸ substituents. In some instances, R¹⁷ is H. In some instances, R¹⁷ is C₁-C₆-alkyl. For example, in some cases, R¹⁷ is methyl or ethyl. In some instances, R¹⁷ is C₁-C₆-alkoxy. For example, in some cases, R¹⁷ is methoxy. In some instances, R¹⁷ is 5- to 10-membered aryl. In some instances, R¹⁷ is S(O)₂—C₁-C₆-alkyl. For example, in some cases, R¹⁷ is S(O)₂-methyl.

In some instances of R¹⁷, each alkyl and aryl is optionally substituted with from 1 to 2 R¹⁸ substituents. In some instances, R¹⁷ is not substituted. In some instances, R¹⁷ is substituted with from 1 to 2 R¹⁸ substituents. In some instances, R¹⁷ is substituted with one R¹⁸ substituent. In some instances, R¹⁷ is substituted with two R¹⁸ substituents.

In certain embodiments, each R¹⁸ is independently selected from OH, oxo, halo, cyano, C₁-C₆-alkyl, C₁-C₆-haloalkyl, C₁-C₆-alkoxy, C₃-C₈-cycloalkyl, 3- to 8-membered heterocycloalkyl, 5- to 10-membered aryl, 5- to 10-membered heteroaryl, NR¹⁹R²⁰C(O)NR¹⁹R²⁰, C(O)OR²⁰, and S(O)₂NR¹⁹R²⁰. In some instances, R¹⁸ is OH. In some instances, R¹⁸ is oxo. In some instances, R¹⁸ is halo (e.g., F, Cl, Br or I). In some instances, R¹⁸ is cyano. In some instances, R¹⁸ is C₁-C₆-alkyl. For example, in some cases, R¹⁸ is methyl. In some instances, R¹⁸ is C₁-C₆-haloalkyl. In some instances, R¹⁸ is C₁-C₆-alkoxy. For example, in some cases, R¹⁸ is methoxy. In some instances, R¹⁸ is C₃-C₈-cycloalkyl. In some instances, R¹⁸ is 3- to 8-membered heterocycloalkyl. In some instances, R¹⁸ is 5- to 10-membered aryl. In some instances, R¹⁸ is 5- to 10-membered heteroaryl. In some instances, R¹⁸ is NR¹⁹R²⁰. For example, in some cases, R¹⁸ is NH₂, NHCH₃ or N(CH₃)₂. In some instances, R¹⁸ is C(O)NR¹⁹R²⁰. For example, in some cases, R¹⁸ is C(O)NH₂, C(O)NHCH₃ or C(O)N(CH₃)₂. In some instances, R¹⁸ is C(O)OR²⁰. For example, in some cases, R¹⁸ is COOH. In some instances, R¹⁸ is S(O)₂NR¹⁹R²⁰. For example, in some cases, R¹⁸ is S(O)₂NH₂ or S(O)₂N(CH₃)₂.

In certain embodiments, each of R¹⁹ and R²⁰ is independently selected from H and C₁-C₆-alkyl. In some instances, R¹⁹ is H. In some instances, R¹⁹ is C₁-C₆-alkyl. For example, in some cases, R¹⁹ is methyl. In some instances, R²⁰ is H. In some instances, R²⁰ is C₁-C₆-alkyl. For example, in some cases, R²⁰ is methyl.

In some instances, R¹⁴ is C₁-C₆-alkoxy substituted with one R¹⁵ substituent. In these embodiments, when the alkoxy of R¹⁴ is substituted with one R¹⁵ substituent, R¹⁵ is 3- to 8-membered heterocycloalkyl, wherein heterocycloalkyl is optionally substituted with 1 to 2 R¹⁶ substituents independently selected from OH, halo, and C₁-C₆-alkyl. For example, in some cases, R¹⁵ is azetidinyl, pyrrolidinyl, piperidinyl, piperazinyl, morpholinyl, tetrahydro-2H-pyranyl or thiomorpholinyl optionally substituted with 1 to 2 R¹⁶ substituents independently selected from OH, halo, and C₁-C₆-alkyl. In some instances, R¹⁵ is azetidinyl, pyrrolidinyl, piperidinyl, piperazinyl, morpholinyl, tetrahydro-2H-pyranyl or thiomorpholinyl. In some cases, R¹⁵ is azetidinyl, pyrrolidinyl, piperidinyl, piperazinyl, morpholinyl, tetrahydro-2H-pyranyl or thiomorpholinyl substituted with one R¹⁶ substituent that is OH, halo, or C₁-C₆-alkyl. In some instances, R¹⁶ is OH. In some instances, R¹⁶ is halo (F, Cl, Br or I). In some instances, R¹⁶ is C₁-C₆-alkyl. For example, in some cases, R¹⁶ is methyl.

In some instances, m is 0 and R¹⁴ is C₁-C₆-alkoxy substituted with one R¹⁵ substituent. In these embodiments, when the alkoxy of R¹⁴ is substituted with one R¹⁵ substituent, R¹⁵ is 3- to 8-membered heterocycloalkyl, wherein heterocycloalkyl is optionally substituted with 1 to 2 R¹⁶ substituents independently selected from OH, halo, and C₁-C₆-alkyl. For example, in some cases, R¹¹ is azetidinyl, pyrrolidinyl, piperidinyl, piperazinyl, morpholinyl, tetrahydro-2H-pyranyl or thiomorpholinyl optionally substituted with 1 to 2 R¹⁶ substituents independently selected from OH, halo, and C₁-C₆-alkyl. In some instances, R¹⁵ is azetidinyl, pyrrolidinyl, piperidinyl, piperazinyl, morpholinyl, tetrahydro-2H-pyranyl or thiomorpholinyl. In some cases, R¹⁵ is azetidinyl, pyrrolidinyl, piperidinyl, piperazinyl, morpholinyl, tetrahydro-2H-pyranyl or thiomorpholinyl substituted with one R¹⁶ substituent that is OH, halo, or C₁-C₆-alkyl. In some instances, R¹⁶ is OH. In some instances, R¹⁶ is halo (F, Cl, Br or I). In some instances, R¹⁶ is C₁-C₆-alkyl. For example, in some cases, R¹⁶ is methyl.

In certain embodiments, R¹⁴ is C₁-C₆-alkoxy substituted with two R¹¹ substituents. In these embodiments, when the alkoxy of R¹⁴ is substituted with two R¹¹ substituents, the first R¹⁵ substituent is oxo and the second R¹⁵ substituent is selected from OH, 3- to 8-membered heterocycloalkyl, and NR¹⁷R¹⁷, wherein heterocycloalkyl is optionally substituted with 1 to 2 R¹⁶ substituents, wherein each R¹⁷ is independently selected from H, C₁-C₆-alkyl, and C₁-C₆-alkoxy, and each R¹⁶ is independently selected from OH, halo, and C₁-C₆-alkyl. In some instances, the first R¹⁵ substituent is oxo and the second R¹⁵ substituent is OH. In some embodiments, the first R¹⁵ substituent is oxo and the second R¹⁵ substituent is 3- to 8-membered heterocycloalkyl. For example, in some cases, heterocycloalkyl is azetidinyl, pyrrolidinyl, piperidinyl, piperazinyl, morpholinyl, tetrahydro-2H-pyranyl or thiomorpholinyl. In some instances, heterocycloalkyl is azetidinyl, pyrrolidinyl, piperidinyl, piperazinyl, morpholinyl, tetrahydro-2H-pyranyl or thiomorpholinyl substituted with one substituent that is OH, halo, or C₁-C₆-alkyl. In some instances, the first R¹⁵ substituent is oxo and the second R¹⁵ substituent is NR¹⁷R¹⁷ wherein each R¹⁷ is independently selected from H, C₁-C₆-alkyl, and C₁-C₆-alkoxy.

In certain embodiments, m is 0 and R¹⁴ is C₁-C₆-alkoxy substituted with two R¹⁵ substituents. In these embodiments, when the alkoxy of R¹⁴ is substituted with two R¹⁵ substituents, the first R¹⁵ substituent is oxo and the second R¹⁵ substituent is selected from OH, 3- to 8-membered heterocycloalkyl, and NR¹⁷R¹⁷, wherein heterocycloalkyl is optionally substituted with 1 to 2 R¹⁶ substituents, wherein each R¹⁷ is independently selected from H, C₁-C₆-alkyl, and C₁-C₆-alkoxy, and each R¹⁶ is independently selected from OH, halo, and C₁-C₆-alkyl. In some instances, the first R¹⁵ substituent is oxo and the second R¹⁵ substituent is OH. In some embodiments, the first R¹⁵ substituent is oxo and the second R¹⁵ substituent is 3- to 8-membered heterocycloalkyl. For example, in some cases, heterocycloalkyl is azetidinyl, pyrrolidinyl, piperidinyl, piperazinyl, morpholinyl, tetrahydro-2H-pyranyl or thiomorpholinyl. In some instances, heterocycloalkyl is azetidinyl, pyrrolidinyl, piperidinyl, piperazinyl, morpholinyl, tetrahydro-2H-pyranyl or thiomorpholinyl substituted with one substituent that is OH, halo, or C₁-C₆-alkyl. In some instances, the first R¹⁵ substituent is oxo and the second R¹⁵ substituent is NR¹⁷R¹⁷ wherein each R¹⁷ is independently selected from H, C₁-C₆-alkyl, and C₁-C₆-alkoxy.

Compounds of the present disclosure (e.g., compounds of formulae (II), (IIa), (IIb), (IIc), (IId) or (IIe) as described herein) also include an enantiomer, a mixture of enantiomers, a mixture of two or more diastereomers, a tautomer, a mixture of two or more tautomers, or an isotopic variant thereof.

In addition, compounds of the present disclosure (e.g., compounds of formulae (II), (IIa), (IIb), (IIc), (IId) or (IIe) as described herein) also include a pharmaceutically acceptable salt, solvate, or hydrate thereof.

In certain embodiments, compounds of the present disclosure (e.g., compounds that find use in the methods of the present disclosure) include compounds selected from:

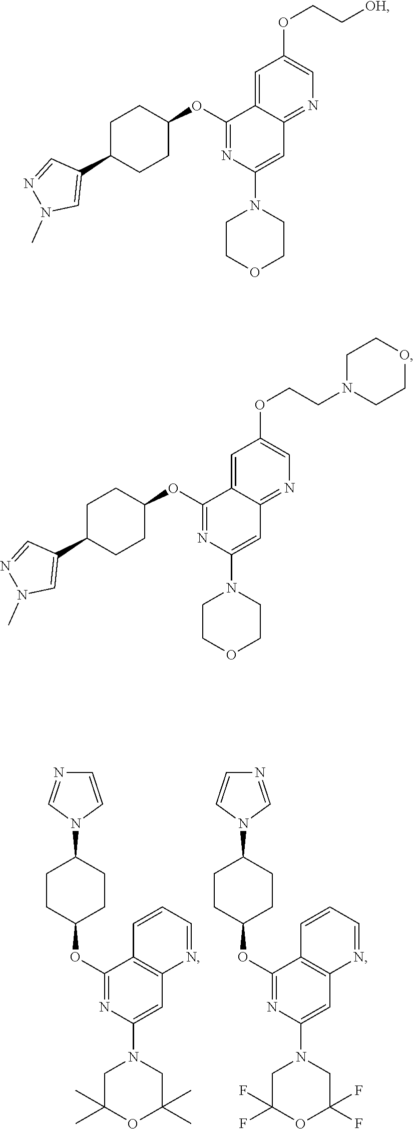

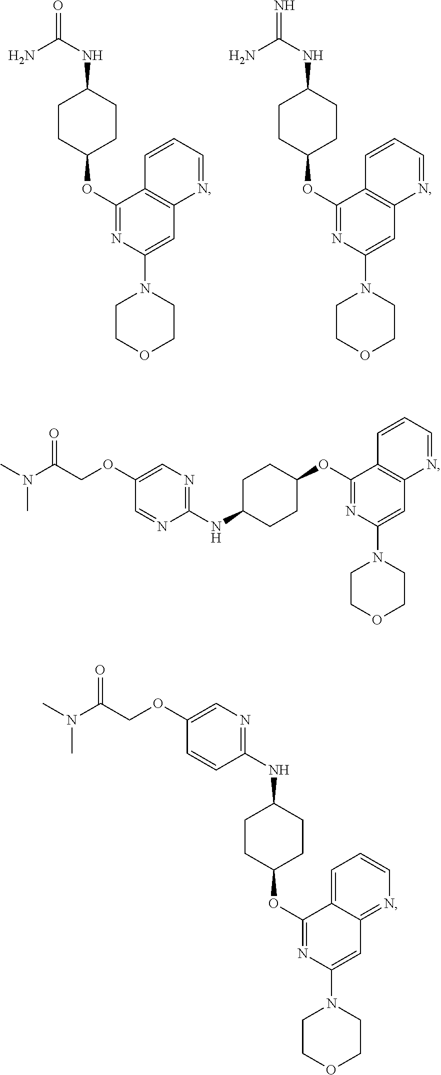 [SMILES: NC(=O)N[C@H]1CC[C@@H](Oc2nc(N3CCOCC3)cc3ncccc23)CC1 ; N=C(N)N[C@H]1CC[C@@H](Oc2nc(N3CCOCC3)cc3ncccc23)CC1 ; CN(C)C(=O)COc1cnc(N[C@H]2CC[C@@H](Oc3nc(N4CCOCC4)cc4ncccc34)CC2)nc1 ; CN(C)C(=O)COc1ccc(N[C@H]2CC[C@@H](Oc3nc(N4CCOCC4)cc4ncccc34)CC2)nc1]

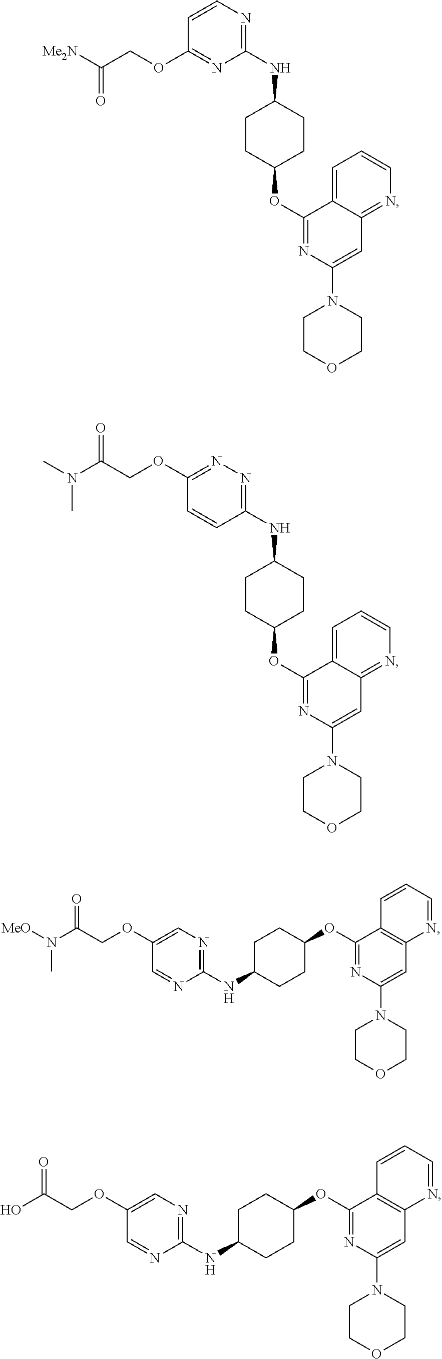 [SMILES: CN(C)C(=O)COc1ccnc(N[C@H]2CC[C@@H](Oc3nc(N4CCOCC4)cc4ncccc34)CC2)n1 ; CN(C)C(=O)COc1ccc(N[C@H]2CC[C@@H](Oc3nc(N4CCOCC4)cc4ncccc34)CC2)nn1 ; CON(C)C(=O)COc1cnc(N[C@H]2CC[C@@H](Oc3nc(N4CCOCC4)cc4ncccc34)CC2)nc1 ; O=C(O)COc1cnc(N[C@H]2CC[C@@H](Oc3nc(N4CCOCC4)cc4ncccc34)CC2)nc1]

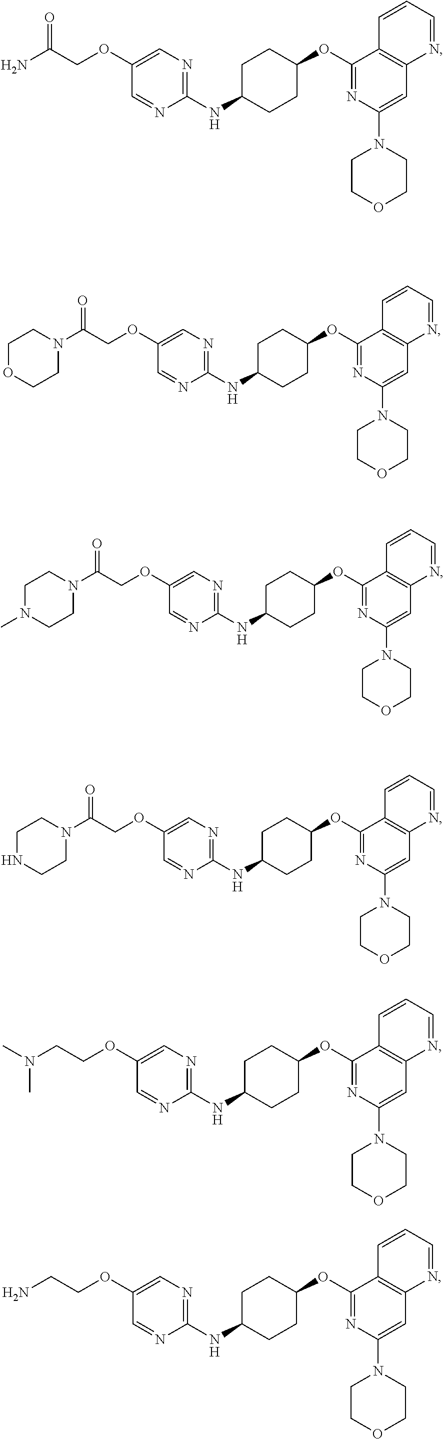

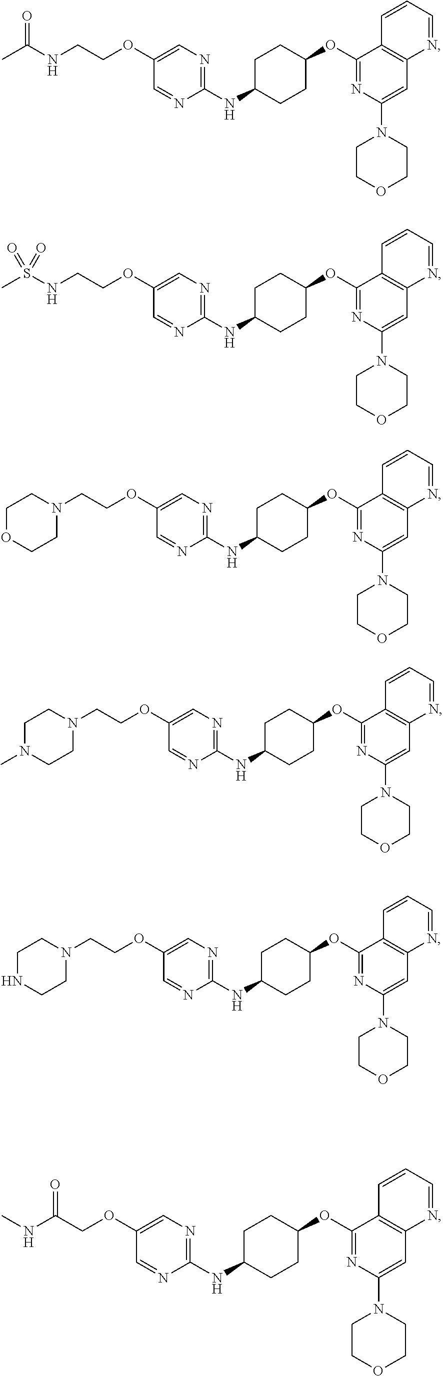

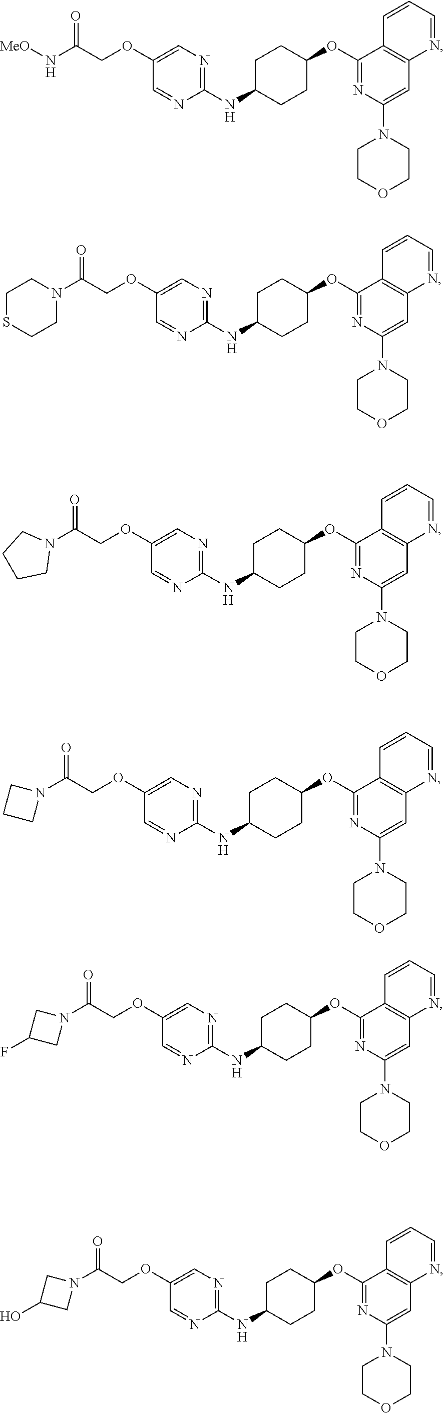 [SMILES: CONC(=O)COc1cnc(N[C@H]2CC[C@@H](Oc3nc(N4CCOCC4)cc4ncccc34)CC2)nc1 ; O=C(COc1cnc(N[C@H]2CC[C@@H](Oc3nc(N4CCOCC4)cc4ncccc34)CC2)nc1)N1CCSCC1 ; O=C(COc1cnc(N[C@H]2CC[C@@H](Oc3nc(N4CCOCC4)cc4ncccc34)CC2)nc1)N1CCCC1 ; O=C(COc1cnc(N[C@H]2CC[C@@H](Oc3nc(N4CCOCC4)cc4ncccc34)CC2)nc1)N1CCC1 ; O=C(COc1cnc(N[C@H]2CC[C@@H](Oc3nc(N4CCOCC4)cc4ncccc34)CC2)nc1)N1CC(F)C1 ; O=C(COc1cnc(N[C@H]2CC[C@@H](Oc3nc(N4CCOCC4)cc4ncccc34)CC2)nc1)N1CC(O)C1]

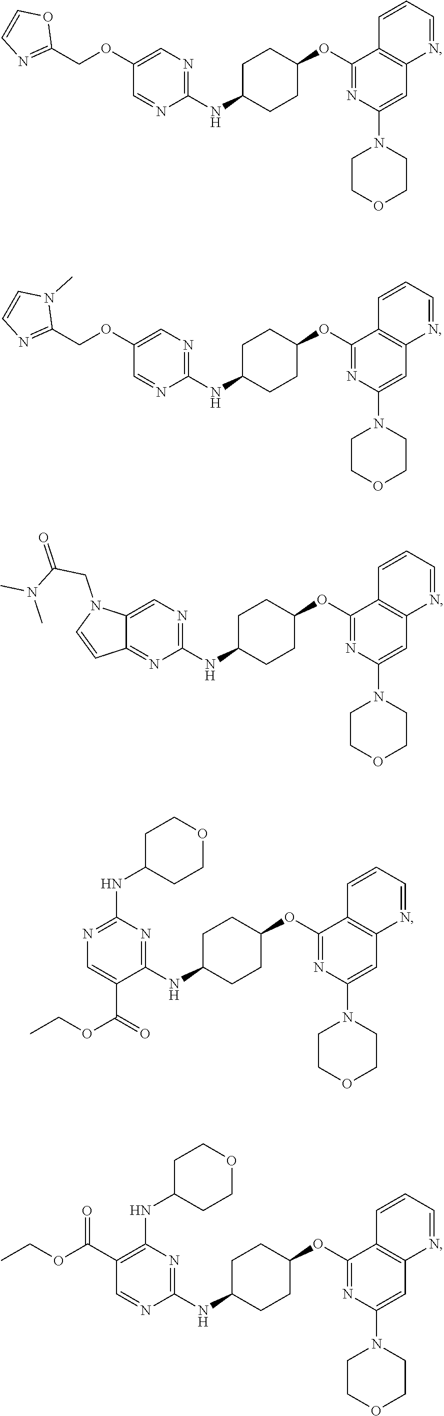 [SMILES: c1cnc2cc(N3CCOCC3)nc(O[C@H]3CC[C@@H](Nc4ncc(OCc5ncco5)cn4)CC3)c2c1 ; Cn1ccnc1COc1cnc(N[C@H]2CC[C@@H](Oc3nc(N4CCOCC4)cc4ncccc34)CC2)nc1 ; CN(C)C(=O)Cn1ccc2nc(N[C@H]3CC[C@@H](Oc4nc(N5CCOCC5)cc5ncccc45)CC3)ncc21 ; CCOC(=O)c1cnc(NC2CCOCC2)nc1N[C@H]1CC[C@@H](Oc2nc(N3CCOCC3)cc3ncccc23)CC1 ; CCOC(=O)c1cnc(N[C@H]2CC[C@@H](Oc3nc(N4CCOCC4)cc4ncccc34)CC2)nc1NC1CCOCC1]

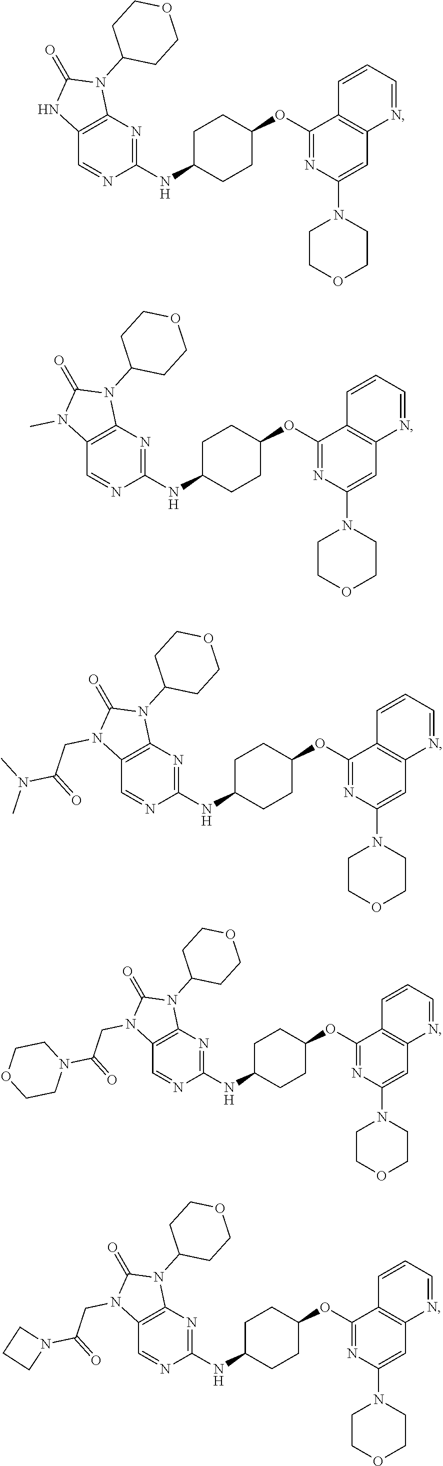 [SMILES: O=c1[nH]c2cnc(N[C@H]3CC[C@@H](Oc4nc(N5CCOCC5)cc5ncccc45)CC3)nc2n1C1CCOCC1 ; Cn1c(=O)n(C2CCOCC2)c2nc(N[C@H]3CC[C@@H](Oc4nc(N5CCOCC5)cc5ncccc45)CC3)ncc21 ; CN(C)C(=O)Cn1c(=O)n(C2CCOCC2)c2nc(N[C@H]3CC[C@@H](Oc4nc(N5CCOCC5)cc5ncccc45)CC3)ncc21 ; O=C(Cn1c(=O)n(C2CCOCC2)c2nc(N[C@H]3CC[C@@H](Oc4nc(N5CCOCC5)cc5ncccc45)CC3)ncc21)N1CCOCC1 ; O=C(Cn1c(=O)n(C2CCOCC2)c2nc(N[C@H]3CC[C@@H](Oc4nc(N5CCOCC5)cc5ncccc45)CC3)ncc21)N1CCC1]

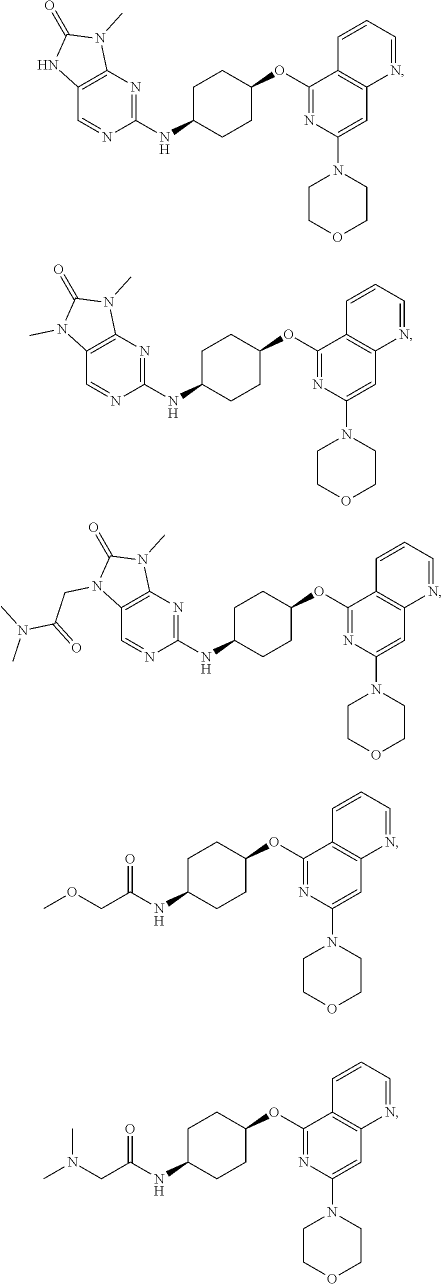

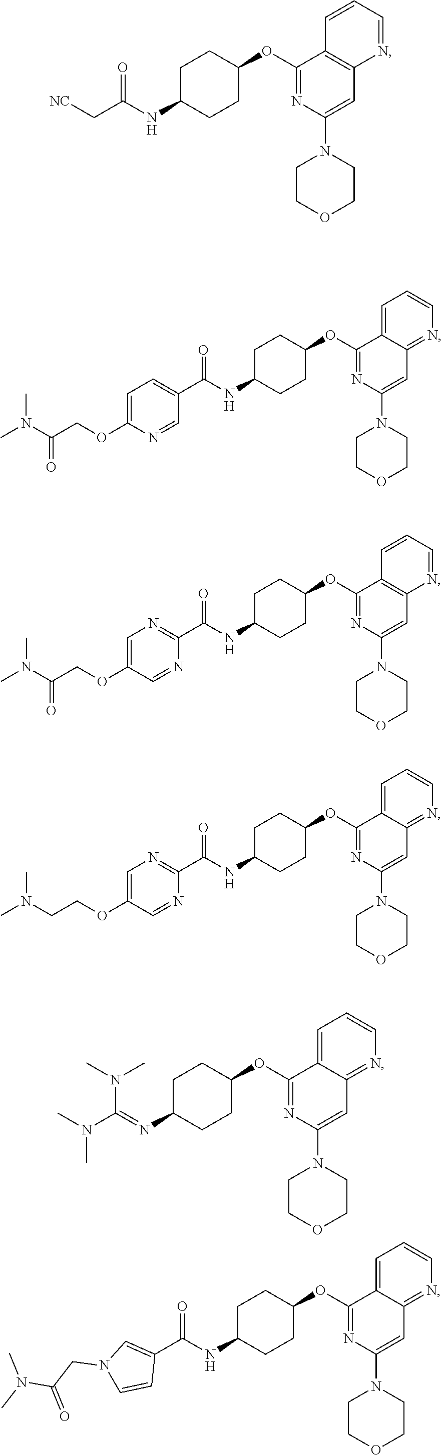 [SMILES: N#CCC(=O)N[C@H]1CC[C@@H](Oc2nc(N3CCOCC3)cc3ncccc23)CC1 ; CN(C)C(=O)COc1ccc(C(=O)N[C@H]2CC[C@@H](Oc3nc(N4CCOCC4)cc4ncccc34)CC2)cn1 ; CN(C)C(=O)COc1cnc(C(=O)N[C@H]2CC[C@@H](Oc3nc(N4CCOCC4)cc4ncccc34)CC2)nc1 ; CN(C)CCOc1cnc(C(=O)N[C@H]2CC[C@@H](Oc3nc(N4CCOCC4)cc4ncccc34)CC2)nc1 ; CN(C)C(=N[C@H]1CC[C@@H](Oc2nc(N3CCOCC3)cc3ncccc23)CC1)N(C)C ; CN(C)C(=O)Cn1ccc(C(=O)N[C@H]2CC[C@@H](Oc3nc(N4CCOCC4)cc4ncccc34)CC2)c1]

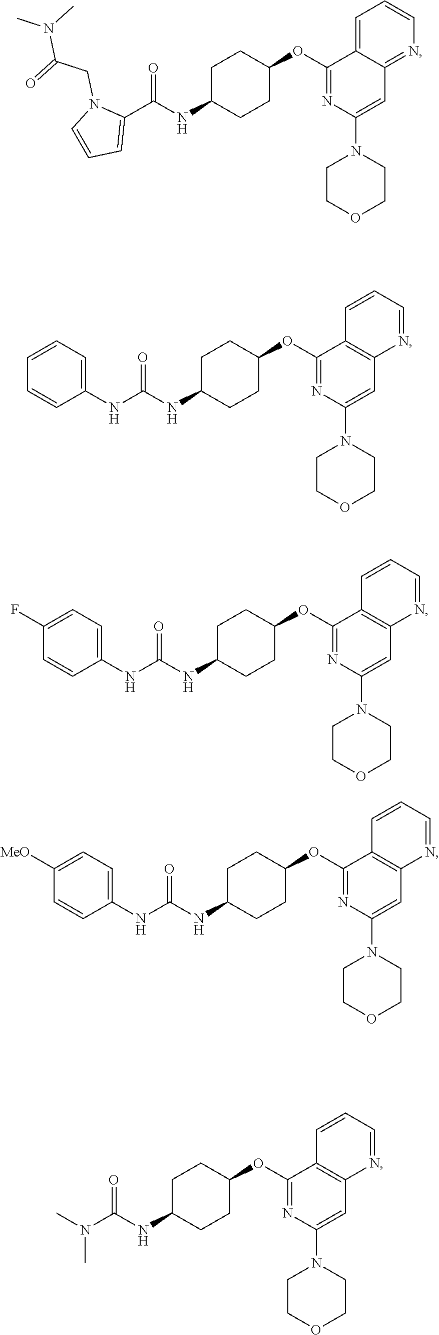

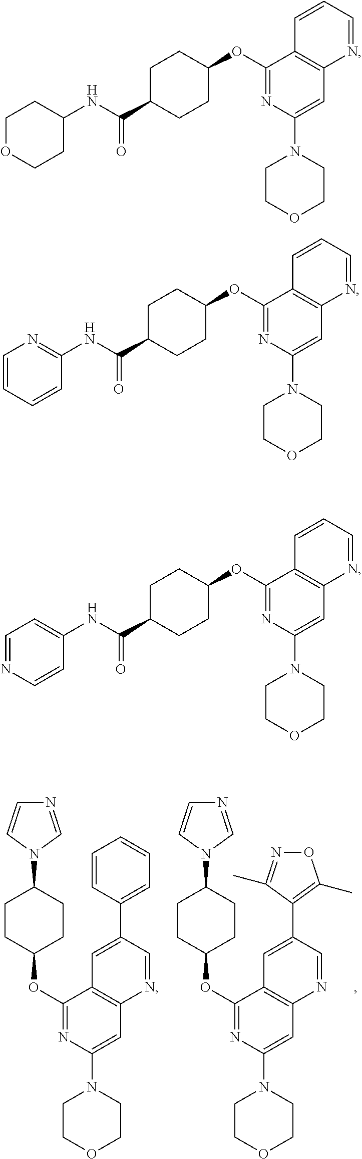

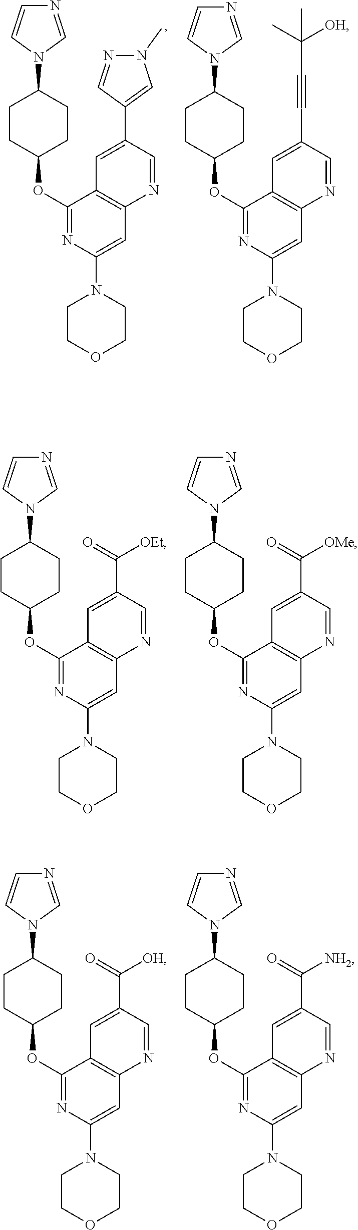

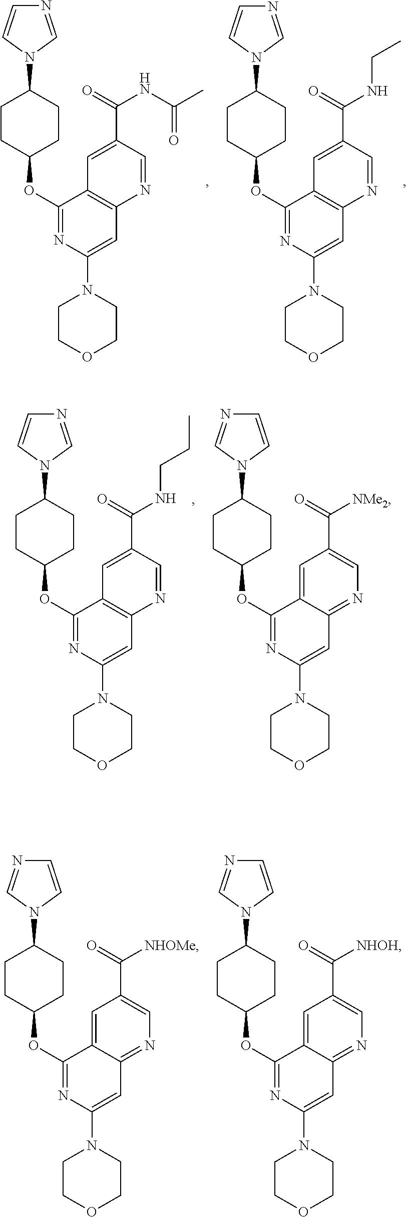

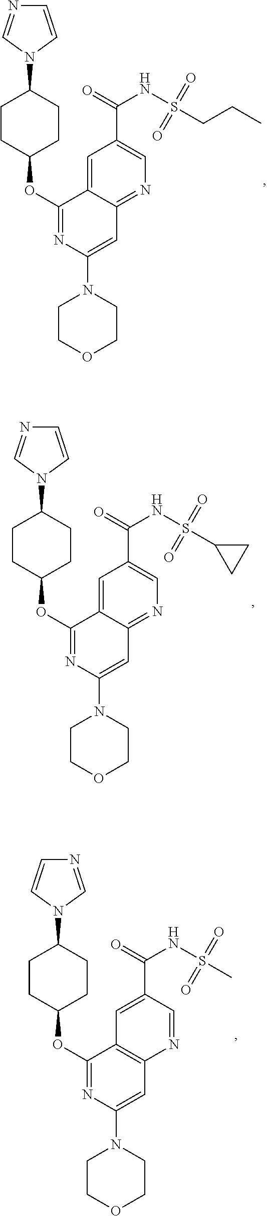 [SMILES: CCCS(=O)(=O)NC(=O)c1cnc2cc(N3CCOCC3)nc(O[C@H]3CC[C@@H](n4ccnc4)CC3)c2c1 ; O=C(NS(=O)(=O)C1CC1)c1cnc2cc(N3CCOCC3)nc(O[C@H]3CC[C@@H](n4ccnc4)CC3)c2c1 ; CS(=O)(=O)NC(=O)c1cnc2cc(N3CCOCC3)nc(O[C@H]3CC[C@@H](n4ccnc4)CC3)c2c1]

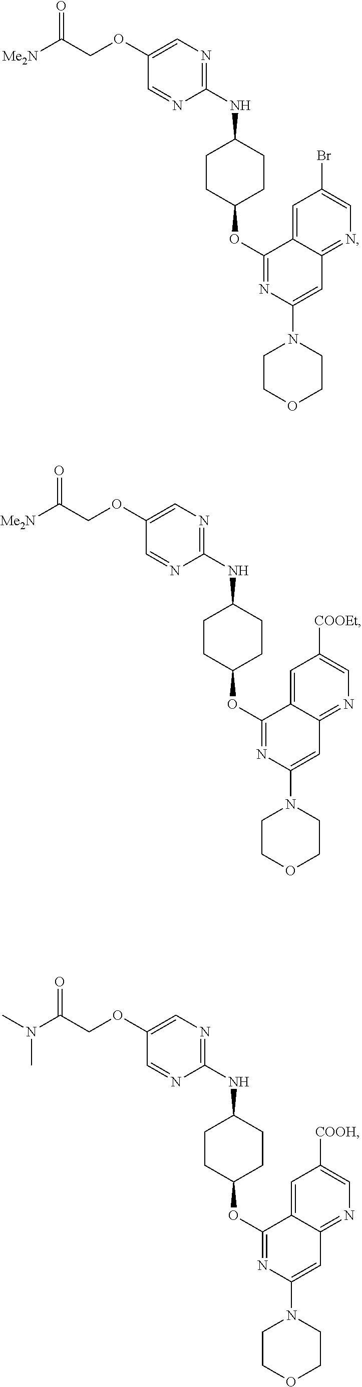 [SMILES: CN(C)C(=O)COc1cnc(N[C@H]2CC[C@@H](Oc3nc(N4CCOCC4)cc4ncc(Br)cc34)CC2)nc1 ; CCOC(=O)c1cnc2cc(N3CCOCC3)nc(O[C@H]3CC[C@@H](Nc4ncc(OCC(=O)N(C)C)cn4)CC3)c2c1 ; CN(C)C(=O)COc1cnc(N[C@H]2CC[C@@H](Oc3nc(N4CCOCC4)cc4ncc(C(=O)O)cc34)CC2)nc1]

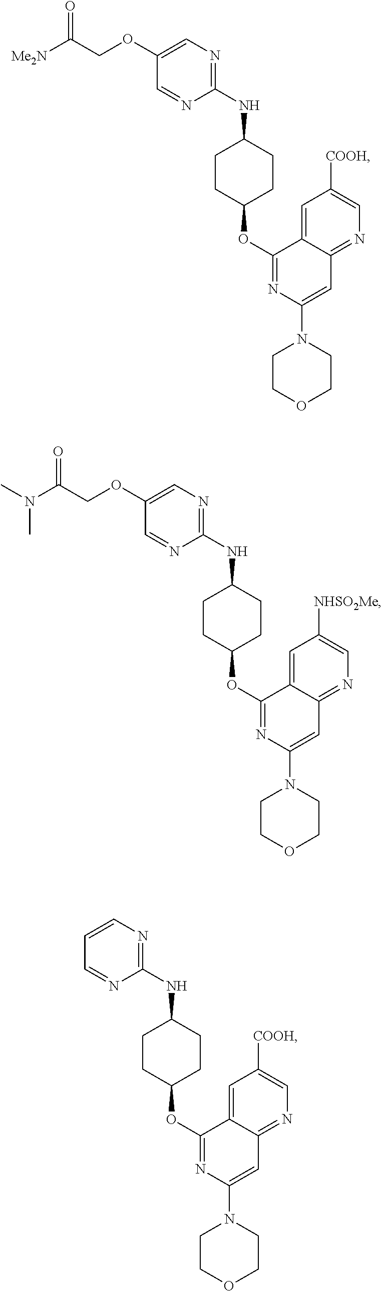

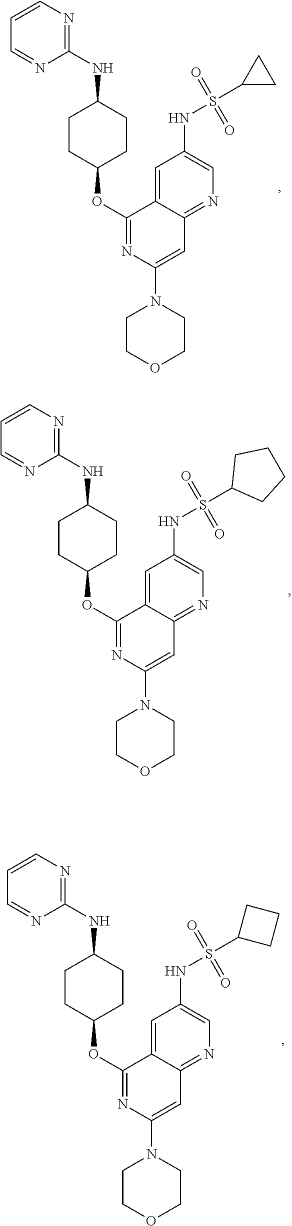 [SMILES: O=S(=O)(Nc1cnc2cc(N3CCOCC3)nc(O[C@H]3CC[C@@H](Nc4ncccn4)CC3)c2c1)C1CC1 ; O=S(=O)(Nc1cnc2cc(N3CCOCC3)nc(O[C@H]3CC[C@@H](Nc4ncccn4)CC3)c2c1)C1CCCC1 ; O=S(=O)(Nc1cnc2cc(N3CCOCC3)nc(O[C@H]3CC[C@@H](Nc4ncccn4)CC3)c2c1)C1CCC1]

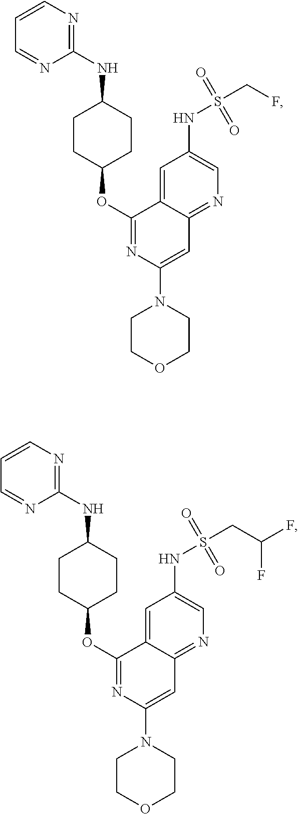 [SMILES: O=S(=O)(CF)Nc1cnc2cc(N3CCOCC3)nc(O[C@H]3CC[C@@H](Nc4ncccn4)CC3)c2c1 ; O=S(=O)(CC(F)F)Nc1cnc2cc(N3CCOCC3)nc(O[C@H]3CC[C@@H](Nc4ncccn4)CC3)c2c1]

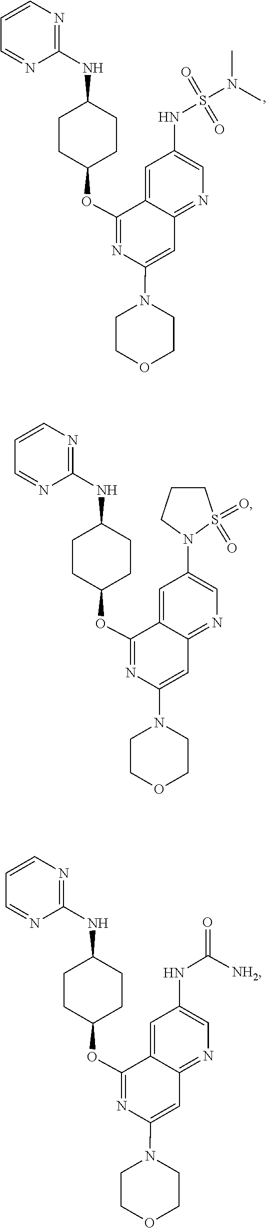 [SMILES: CN(C)S(=O)(=O)Nc1cnc2cc(N3CCOCC3)nc(O[C@H]3CC[C@@H](Nc4ncccn4)CC3)c2c1 ; O=S1(=O)CCCN1c1cnc2cc(N3CCOCC3)nc(O[C@H]3CC[C@@H](Nc4ncccn4)CC3)c2c1 ; NC(=O)Nc1cnc2cc(N3CCOCC3)nc(O[C@H]3CC[C@@H](Nc4ncccn4)CC3)c2c1]

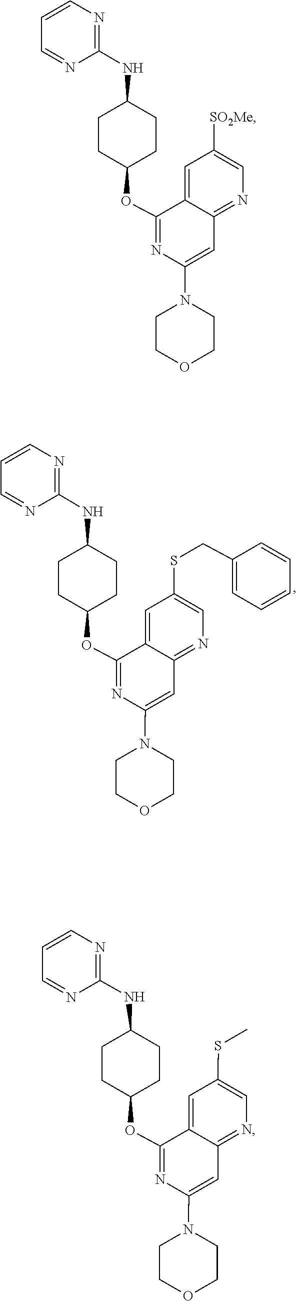 [SMILES: CS(=O)(=O)c1cnc2cc(N3CCOCC3)nc(O[C@H]3CC[C@@H](Nc4ncccn4)CC3)c2c1 ; c1ccc(CSc2cnc3cc(N4CCOCC4)nc(O[C@H]4CC[C@@H](Nc5ncccn5)CC4)c3c2)cc1 ; CSc1cnc2cc(N3CCOCC3)nc(O[C@H]3CC[C@@H](Nc4ncccn4)CC3)c2c1]

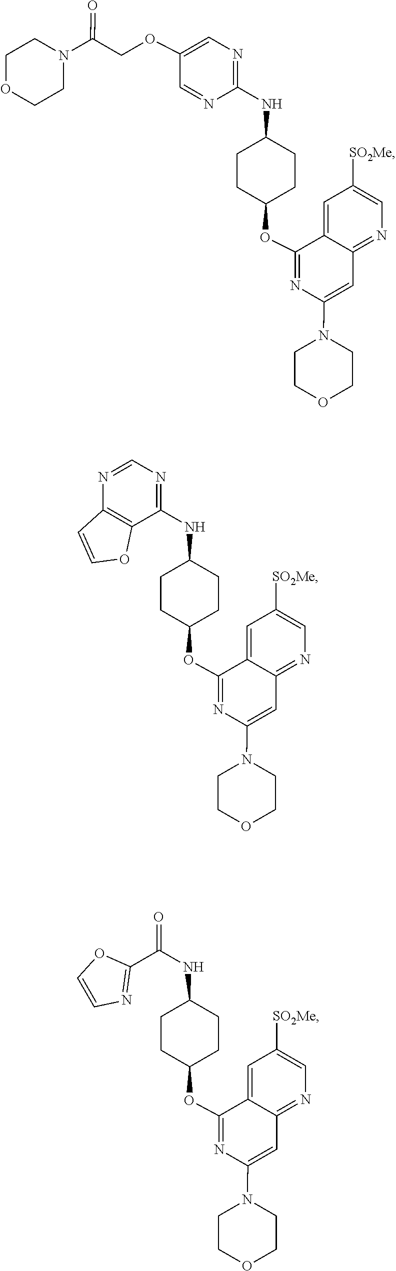 [SMILES: CS(=O)(=O)c1cnc2cc(N3CCOCC3)nc(O[C@H]3CC[C@@H](Nc4ncc(OCC(=O)N5CCOCC5)cn4)CC3)c2c1 ; CS(=O)(=O)c1cnc2cc(N3CCOCC3)nc(O[C@H]3CC[C@@H](Nc4ncnc5ccoc45)CC3)c2c1 ; CS(=O)(=O)c1cnc2cc(N3CCOCC3)nc(O[C@H]3CC[C@@H](NC(=O)c4ncco4)CC3)c2c1]

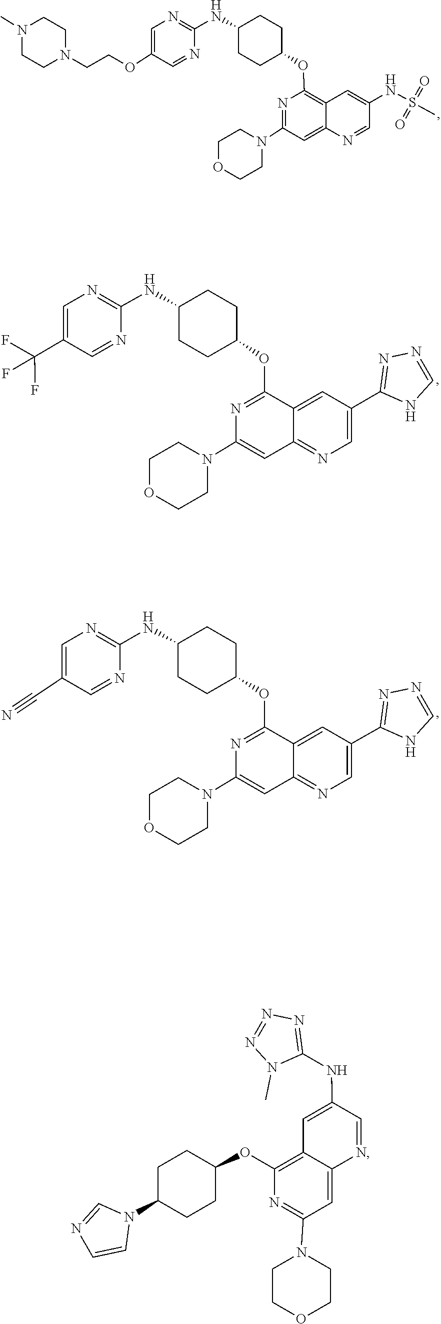

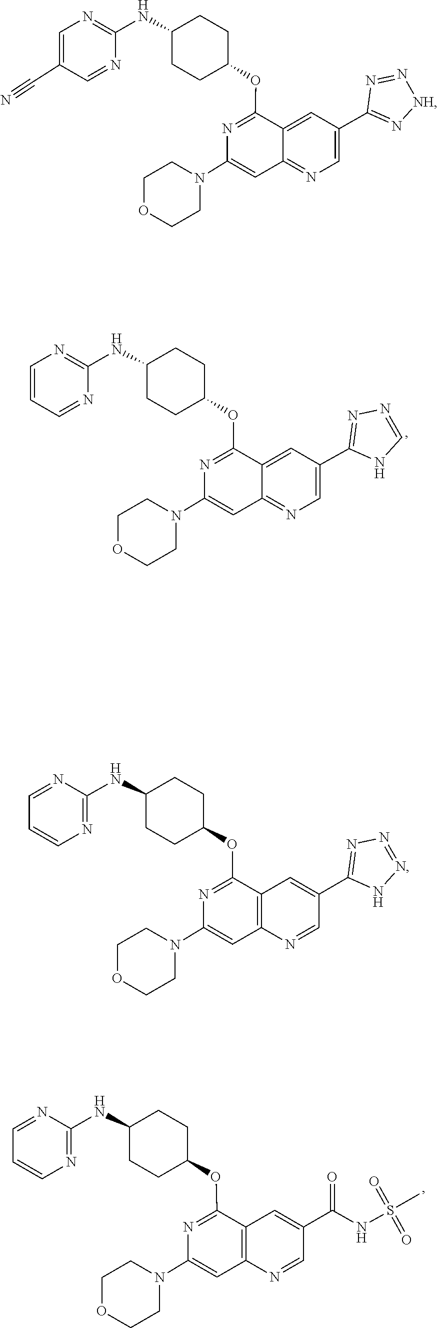 [SMILES: N#Cc1cnc(N[C@H]2CC[C@@H](Oc3nc(N4CCOCC4)cc4ncc(-c5nn[nH]n5)cc34)CC2)nc1 ; c1cnc(N[C@H]2CC[C@@H](Oc3nc(N4CCOCC4)cc4ncc(-c5nnc[nH]5)cc34)CC2)nc1 ; c1cnc(N[C@H]2CC[C@@H](Oc3nc(N4CCOCC4)cc4ncc(-c5nnn[nH]5)cc34)CC2)nc1 ; CS(=O)(=O)NC(=O)c1cnc2cc(N3CCOCC3)nc(O[C@H]3CC[C@@H](Nc4ncccn4)CC3)c2c1]

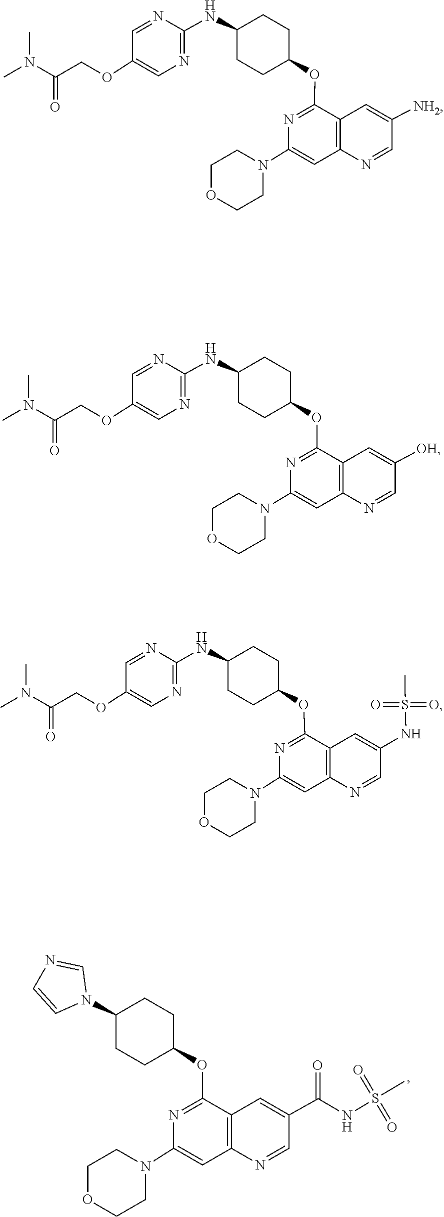 [SMILES: CN(C)C(=O)COc1cnc(N[C@H]2CC[C@@H](Oc3nc(N4CCOCC4)cc4ncc(N)cc34)CC2)nc1 ; CN(C)C(=O)COc1cnc(N[C@H]2CC[C@@H](Oc3nc(N4CCOCC4)cc4ncc(O)cc34)CC2)nc1 ; CN(C)C(=O)COc1cnc(N[C@H]2CC[C@@H](Oc3nc(N4CCOCC4)cc4ncc(NS(C)(=O)=O)cc34)CC2)nc1 ; CS(=O)(=O)NC(=O)c1cnc2cc(N3CCOCC3)nc(O[C@H]3CC[C@@H](n4ccnc4)CC3)c2c1]

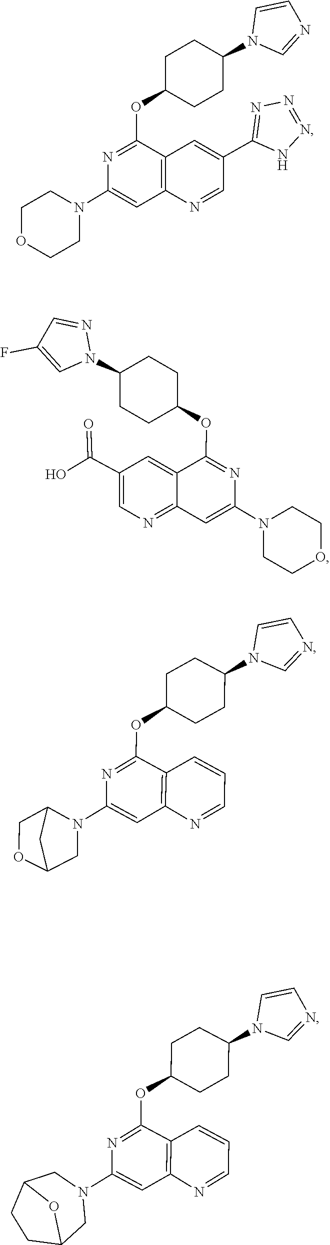

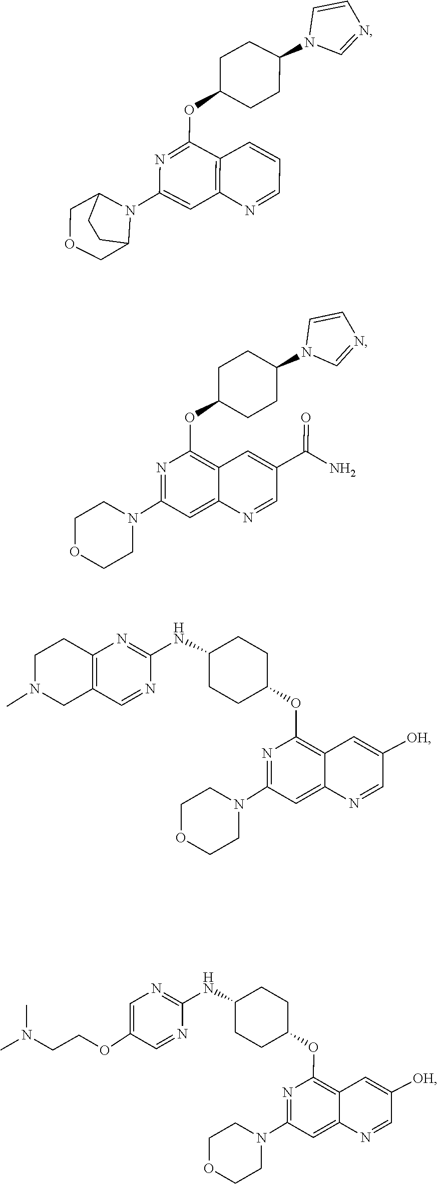 [SMILES: c1cnc2cc(N3C4CCC3COC4)nc(O[C@H]3CC[C@@H](n4ccnc4)CC3)c2c1 ; NC(=O)c1cnc2cc(N3CCOCC3)nc(O[C@H]3CC[C@@H](n4ccnc4)CC3)c2c1 ; CN1CCc2nc(N[C@H]3CC[C@@H](Oc4nc(N5CCOCC5)cc5ncc(O)cc45)CC3)ncc2C1 ; CN(C)CCOc1cnc(N[C@H]2CC[C@@H](Oc3nc(N4CCOCC4)cc4ncc(O)cc34)CC2)nc1]

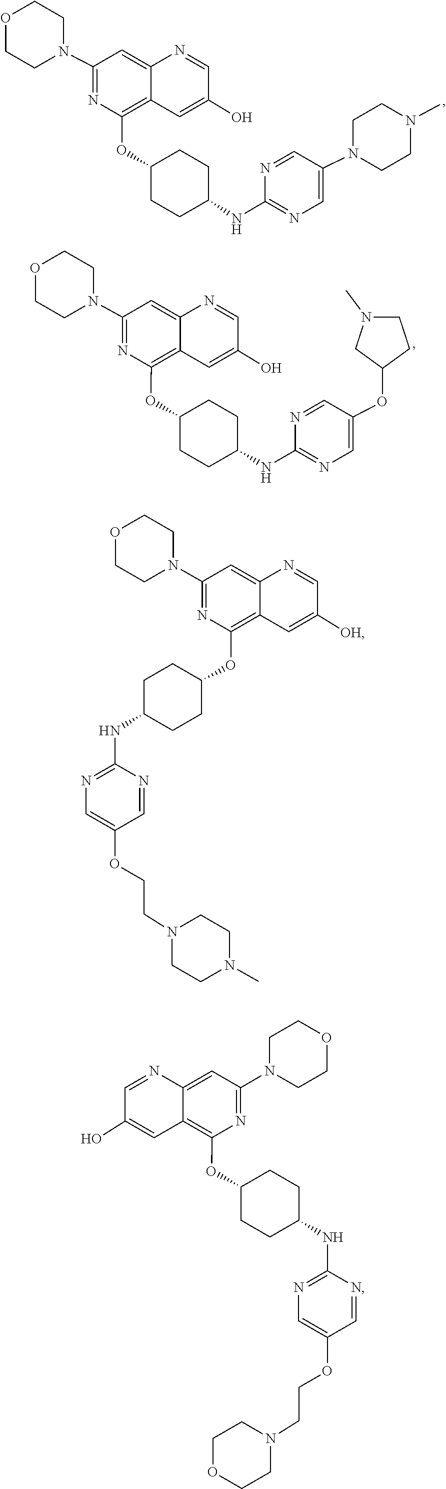 [SMILES: CN1CCN(c2cnc(N[C@H]3CC[C@@H](Oc4nc(N5CCOCC5)cc5ncc(O)cc45)CC3)nc2)CC1 ; CN1CCC(Oc2cnc(N[C@H]3CC[C@@H](Oc4nc(N5CCOCC5)cc5ncc(O)cc45)CC3)nc2)C1 ; CN1CCN(CCOc2cnc(N[C@H]3CC[C@@H](Oc4nc(N5CCOCC5)cc5ncc(O)cc45)CC3)nc2)CC1 ; Oc1cnc2cc(N3CCOCC3)nc(O[C@H]3CC[C@@H](Nc4ncc(OCCN5CCOCC5)cn4)CC3)c2c1]

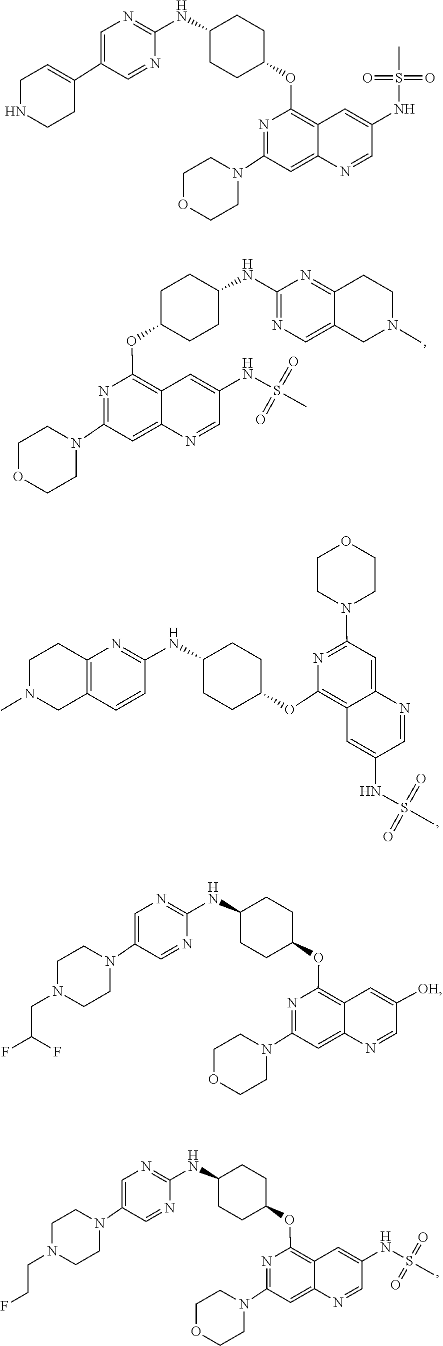

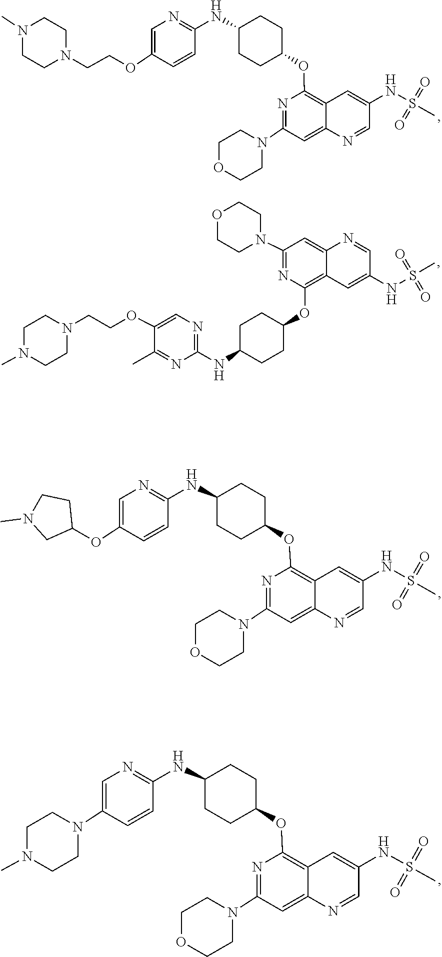

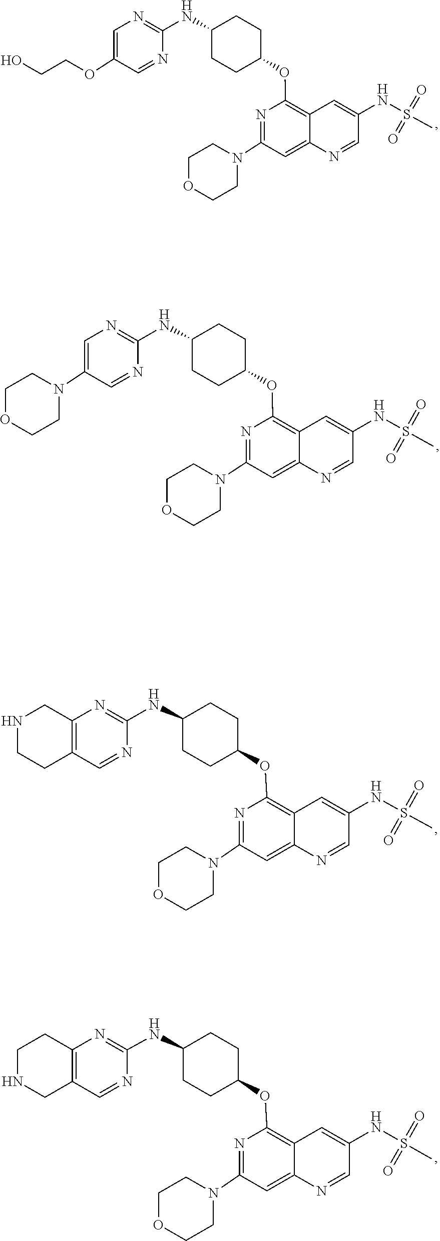 [SMILES: CS(=O)(=O)Nc1cnc2cc(N3CCOCC3)nc(O[C@H]3CC[C@@H](Nc4ncc(OCCO)cn4)CC3)c2c1 ; CS(=O)(=O)Nc1cnc2cc(N3CCOCC3)nc(O[C@H]3CC[C@@H](Nc4ncc(N5CCOCC5)cn4)CC3)c2c1 ; CS(=O)(=O)Nc1cnc2cc(N3CCOCC3)nc(O[C@H]3CC[C@@H](Nc4ncc5c(n4)CNCC5)CC3)c2c1 ; CS(=O)(=O)Nc1cnc2cc(N3CCOCC3)nc(O[C@H]3CC[C@@H](Nc4ncc5c(n4)CCNC5)CC3)c2c1]

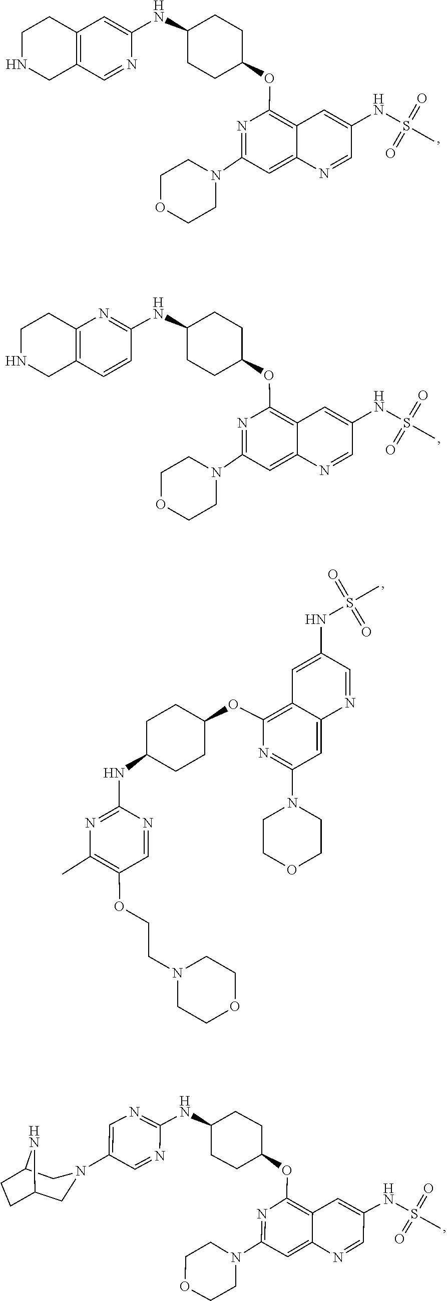

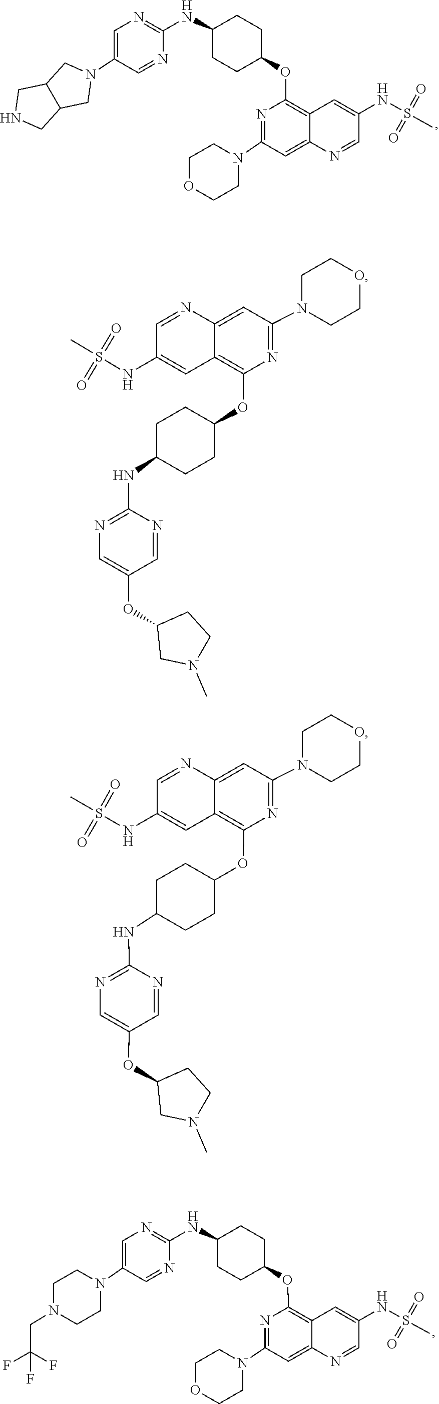

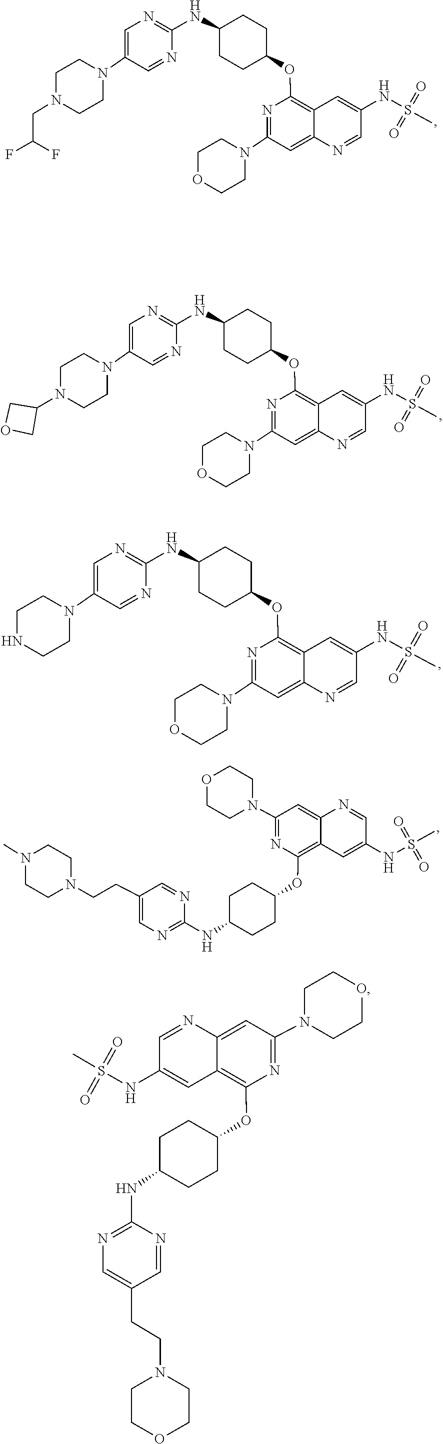

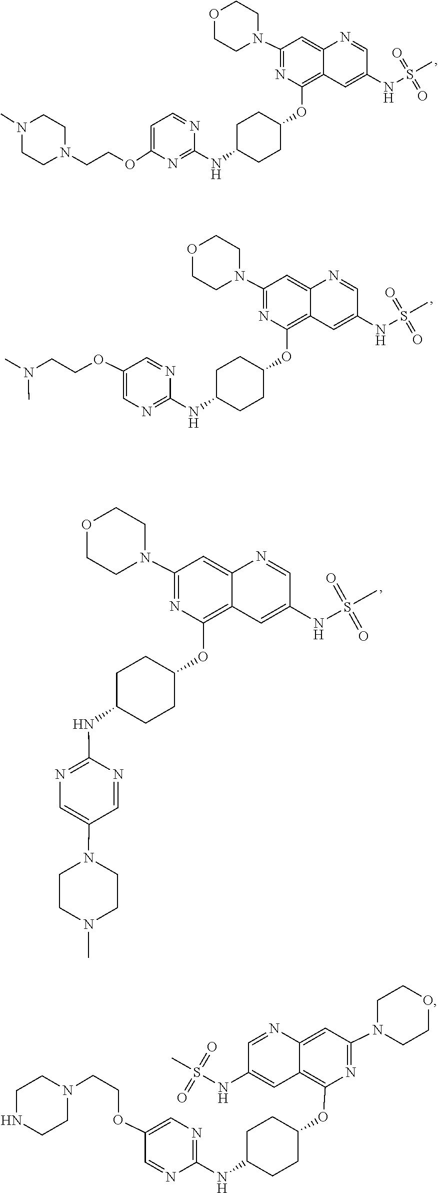

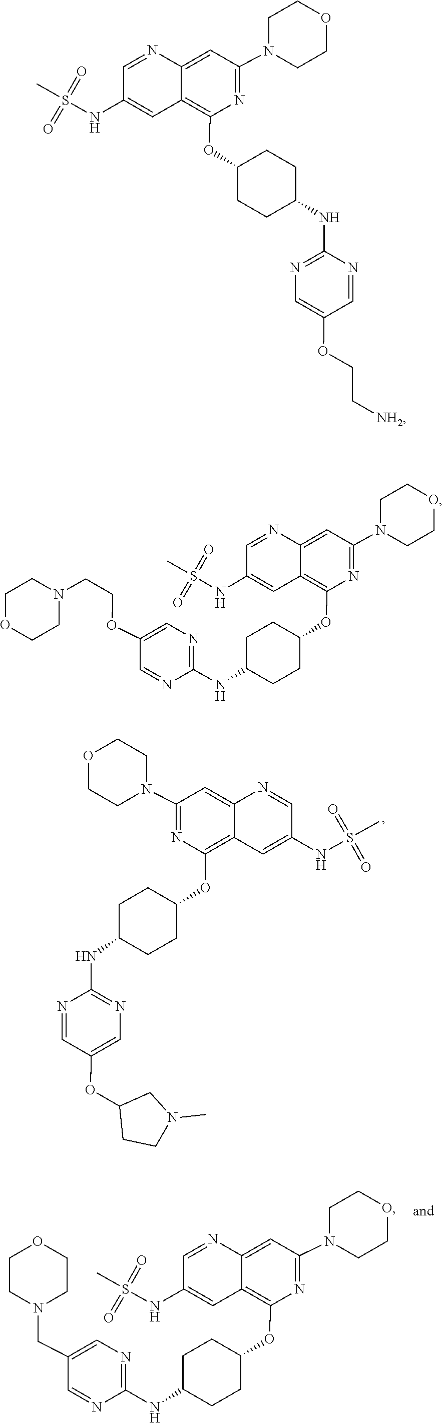

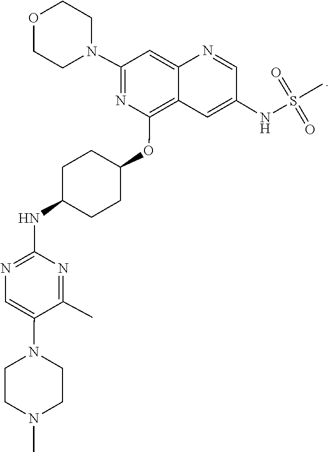 [SMILES: Cc1nc(N[C@H]2CC[C@@H](Oc3nc(N4CCOCC4)cc4ncc(NS(C)(=O)=O)cc34)CC2)ncc1N1CCN(C)CC1]

In certain embodiments, compounds of the present disclosure (e.g., compounds that find use in the methods of the present disclosure) include compounds selected from:

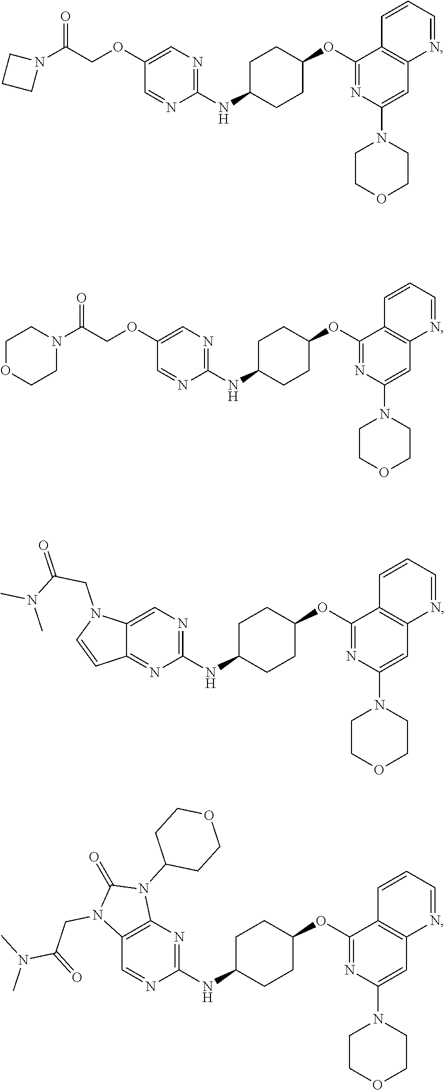 [SMILES: O=C(COc1cnc(N[C@H]2CC[C@@H](Oc3nc(N4CCOCC4)cc4ncccc34)CC2)nc1)N1CCC1 ; O=C(COc1cnc(N[C@H]2CC[C@@H](Oc3nc(N4CCOCC4)cc4ncccc34)CC2)nc1)N1CCOCC1 ; CN(C)C(=O)Cn1ccc2nc(N[C@H]3CC[C@@H](Oc4nc(N5CCOCC5)cc5ncccc45)CC3)ncc21 ; CN(C)C(=O)Cn1c(=O)n(C2CCOCC2)c2nc(N[C@H]3CC[C@@H](Oc4nc(N5CCOCC5)cc5ncccc45)CC3)ncc21]

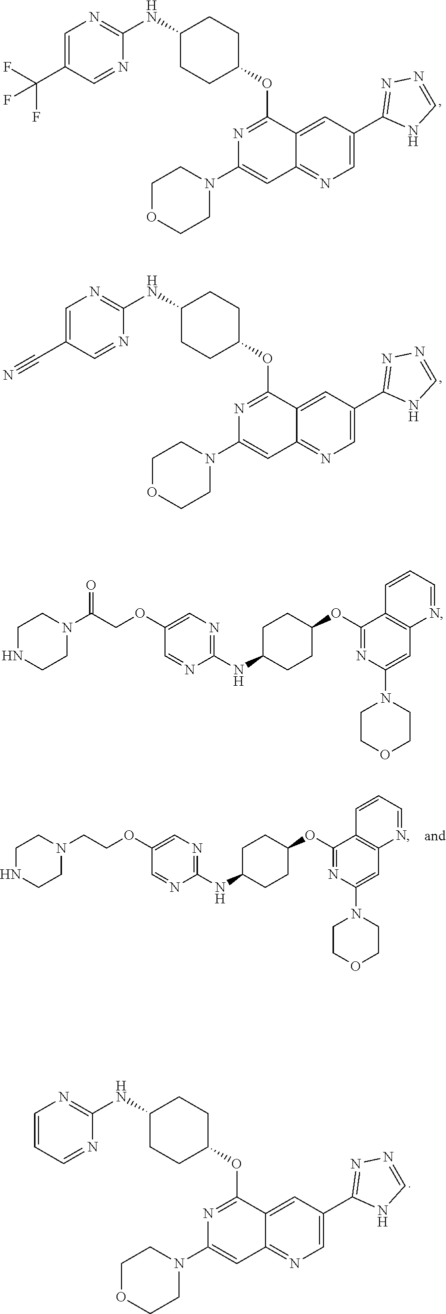

In certain embodiments, compounds of the present disclosure (e.g., compounds that find use in the methods of the present disclosure) include compounds selected from:

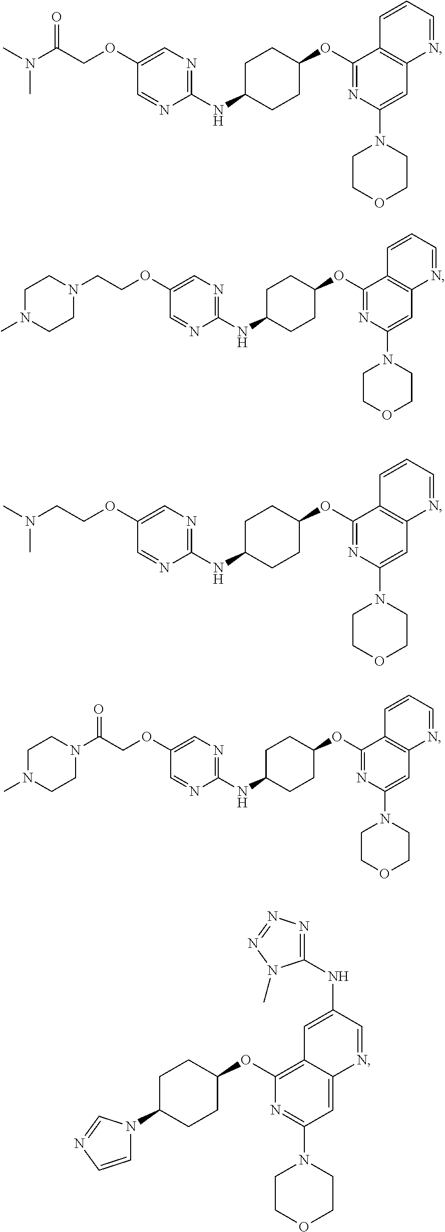 [SMILES: CN(C)C(=O)COc1cnc(N[C@H]2CC[C@@H](Oc3nc(N4CCOCC4)cc4ncccc34)CC2)nc1 ; CN1CCN(CCOc2cnc(N[C@H]3CC[C@@H](Oc4nc(N5CCOCC5)cc5ncccc45)CC3)nc2)CC1 ; CN(C)CCOc1cnc(N[C@H]2CC[C@@H](Oc3nc(N4CCOCC4)cc4ncccc34)CC2)nc1 ; CN1CCN(C(=O)COc2cnc(N[C@H]3CC[C@@H](Oc4nc(N5CCOCC5)cc5ncccc45)CC3)nc2)CC1 ; Cn1nnnc1Nc1cnc2cc(N3CCOCC3)nc(O[C@H]3CC[C@@H](n4ccnc4)CC3)c2c1]

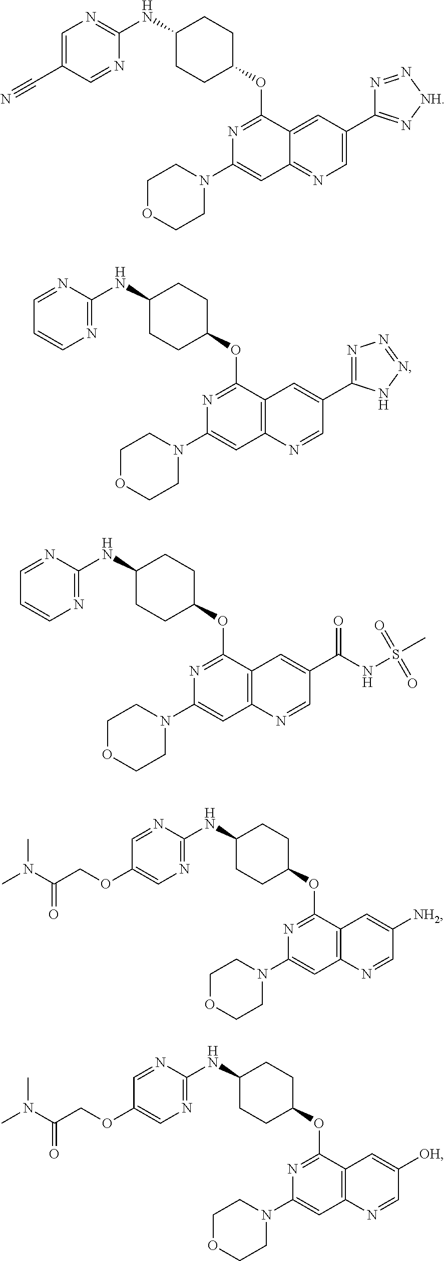

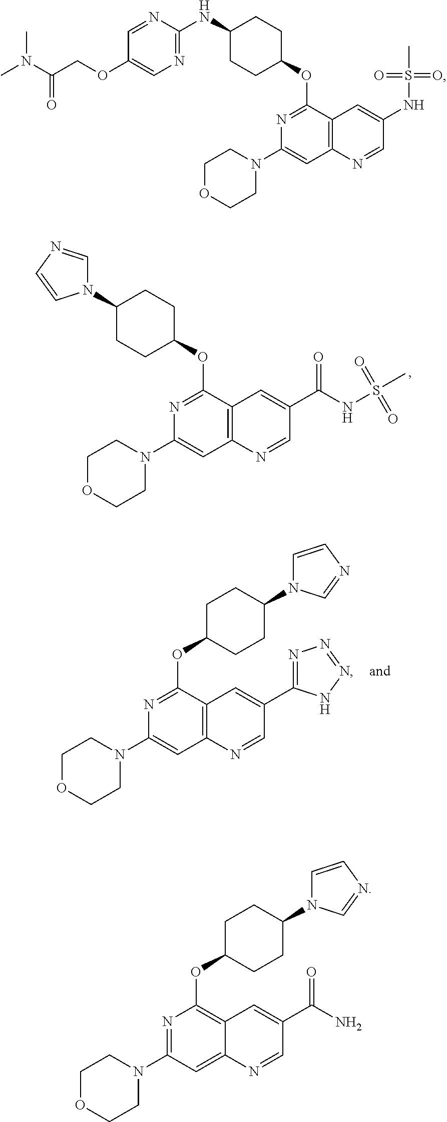 [SMILES: CN(C)C(=O)COc1cnc(N[C@H]2CC[C@@H](Oc3nc(N4CCOCC4)cc4ncc(NS(C)(=O)=O)cc34)CC2)nc1 ; CS(=O)(=O)NC(=O)c1cnc2cc(N3CCOCC3)nc(O[C@H]3CC[C@@H](n4ccnc4)CC3)c2c1 ; c1cn([C@H]2CC[C@@H](Oc3nc(N4CCOCC4)cc4ncc(-c5nnn[nH]5)cc34)CC2)cn1 ; NC(=O)c1cnc2cc(N3CCOCC3)nc(O[C@H]3CC[C@@H](n4ccnc4)CC3)c2c1]

In certain embodiments, the compound is a prodrug of a compound of formula (II), (IIa), (IIb), (IIc), (IId) or (IIe) or a pharmaceutically acceptable salt thereof. In some instances, the prodrug comprises a trigger moiety that releases the compound of formula (II), (IIa), (IIb), (IIc), (IId) or (IIe) under reductive conditions.

In certain embodiments, incorporating a trigger moiety that releases the compound of formula (II), (IIa), (IIb), (IIc), (IId) or (IIe) under reductive conditions allows the selective release of the compounds of the present disclosure in hypoxic tissue, such as occurs within solid tumors. Thus, the prodrugs are hypoxia-activated compounds that may show reduced toxicity by employing two mechanisms for selectivity. First, the compounds may have specificity for hypoxic cells and are therefore expected to exhibit reduced systemic DNA-PK inhibition in oxic cells in the body. Second, the compounds of the present disclosure would only impact cells sustaining DNA-damage resulting from e.g. radiotherapy. This double specificity has the potential to result in a wide safety margin. The compounds of the present disclosure may have specificity for activity in hypoxic cells, or specificity for activation and release of effector compounds by hypoxic cells for activity in these and proximal tissues through diffusion and may therefore be expected to exhibit reduced systemic DNA-PK inhibition in body tissues with little hypoxia.

As described above, R² can be OR⁵, NR⁶R⁷, C(O)NR⁶R⁷ or 5- to 10-membered heteroaryl. For example, R² can be OR⁵. In other instances, R² can be a substituted amino group (NR⁶R⁷). In other instances, R² can be a substituted amide group (C(O)NR⁶R⁷). In other instances, R² can be a substituted 5-membered heteroaryl group, where the heteroaryl includes at least one nitrogen atom in the ring system. In some cases, the heteroaryl ring includes at least two nitrogens in the ring system. For instance, R² may be selected from pyrazole, imidazole 1,2,3-triazole, 1,2,4-triazole and tetrazole. Where R² is a 5-membered heteroaryl that includes at least one nitrogen in the ring system, R² may be attached to the rest of the molecule via the nitrogen (where the heteroaryl group comprises one nitrogen in the ring system) or via one of the nitrogens (where the heteroaryl group comprises two or more nitrogens in the ring system). Alternatively, where R² is a 5-membered heteroaryl that includes at least one nitrogen in the ring system, R² may be attached to the rest of the molecule via a carbon atom. In these compounds, the nitrogen (where the heteroaryl group comprises one nitrogen in the ring system) or one of the nitrogens (where the heteroaryl group comprises two or more nitrogens in the ring system) may be the group to which a trigger moiety is attached to form a prodrug that releases a compound of formula (II), (IIa), (IIb), (IIc), (IId) or (IIe) when subjected to reductive conditions.

In some cases, the compound is a prodrug in which a trigger moiety that releases a compound of formula (II), (IIa), (IIb), (IIc), (IId) or (IIe) under reductive conditions is attached to an oxygen atom of R², such as the oxygen atom in OR⁵. In some instances, when a trigger moiety is attached to OR⁵, the trigger moiety is attached to the oxygen in place of the R⁵ group. As such, in some cases, when a trigger moiety is attached to OR⁵, R⁵ is the trigger moiety. Stated another way, when a trigger moiety is attached to OR⁵, R⁵ can be a bond attached to a trigger moiety.

In some cases, the compound is a prodrug in which a trigger moiety that releases a compound of formula (II), (IIa), (IIb), (IIc), (IId) or (IIe) under reductive conditions is attached to a nitrogen atom of R², such as a nitrogen atom of NR⁶R⁷, C(O)NR⁶R⁷ or 5- to 10-membered heteroaryl (e.g., pyrazole, imidazole 1,2,3-triazole, 1,2,4-triazole and tetrazole). In some instances, when a trigger moiety is attached to NR⁶R⁷ or C(O)NR⁶R⁷, the trigger moiety is attached to the nitrogen in place of the R⁶ group. As such, in some cases, when a trigger moiety is attached to NR⁶R⁷ or C(O)NR⁶R⁷, R⁶ is the trigger moiety. Stated another way, when a trigger moiety is attached to NR⁶R⁷ or C(O)NR⁶R⁷, R⁶ can be a bond attached to a trigger moiety. In some instances where the trigger moiety is attached to NR⁶R⁷ or C(O)NR⁶R⁷, R⁶ is the trigger moiety and R⁷ is S(O)₂—C₁-C₆-alkyl, such as S(O)₂-methyl.

In some cases, the compound is a prodrug in which a trigger moiety that releases a compound of formula (II), (IIa), (IIb), (IIc), (IId) or (IIe) under reductive conditions is attached to an oxygen atom of R², such as an oxygen atom of NR⁶R⁷, where R⁶ is C(O)OR⁵. As such, in these instances, R² can be N(C(O)OR⁵)R⁷. In some instances, when a trigger moiety is attached to C(O)OR⁵, the trigger moiety is attached to the oxygen in place of the R⁵ group. As such, in some cases, when a trigger moiety is attached to C(O)OR⁵, R⁵ is the trigger moiety. Stated another way, when a trigger moiety is attached to C(O)OR⁵, R⁵ can be a bond attached to a trigger moiety. In some instances where the trigger moiety is attached to N(C(O)OR⁵)R⁷, R⁵ is the trigger moiety and R⁷ is S(O)₂—C₁-C₆-alkyl, such as S(O)₂-methyl.

In some cases, the compound is a prodrug in which a trigger moiety that releases a compound of formula (II), (IIa), (IIb), (IIc), (IId) or (IIe) under reductive conditions is attached to a sulfer atom of R², such as the sulfer atom in S(O)₂—NR¹⁰R¹⁰. In some instances, when a trigger moiety is attached to S(O)₂—NR¹⁰R¹⁰, the trigger moiety is attached to the sulfer in place of one of the R¹⁰ groups. As such, in some cases, when a trigger moiety is attached to S(O)₂—NR¹⁰R¹⁰, one of the R¹⁰ groups is the trigger moiety. Stated another way, when a trigger moiety is attached to S(O)₂—NR¹⁰R¹⁰, one of the R¹⁰ groups can be a bond attached to a trigger moiety.

In certain embodiments, the trigger moiety has a structure selected from:

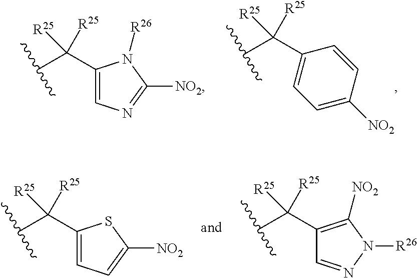

wherein:
each R²⁵ is independently selected from H and C₁-C₆-alkyl; and
R²⁶ is selected from C₁-C₃-alkyl and C₃-C₅-cycloalkyl.

In certain embodiments, each R²⁵ is independently selected from H and C₁-C₆-alkyl. In some instances, R²⁵ is H. In some instances, R²⁵ is C₁-C₆-alkyl. For example, R²⁵ can be methyl. In some instances, both R²⁵ groups are the same (e.g., both R²⁵ groups are H or CH₃). In some instances, the R²⁵ groups are different (e.g., one R²⁵ is H and one R²⁵ is CH₃).

In certain embodiments, R²⁶ is selected from C₁-C₃-alkyl and C₃-C₅-cycloalkyl. In some instances, R²⁶ is C₁-C₃-alkyl. For example, in some cases, R²⁶ is methyl. In some instances, R²⁶ is C₃-C₅-cycloalkyl.

In some instances, the trigger moiety has a structure selected from:

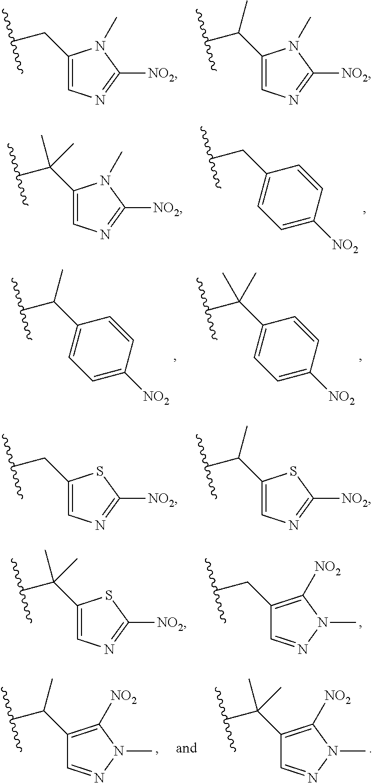

Prodrug compounds of the present disclosure (e.g., prodrug compounds of formulae (II), (IIa), (IIb), (IIc), (IId) or (IIe) as described herein) also include an enantiomer, a mixture of enantiomers, a mixture of two or more diastereomers, a tautomer, a mixture of two or more tautomers, or an isotopic variant thereof.

In addition, prodrug compounds of the present disclosure (e.g., prodrug compounds of formulae (II), (IIa), (IIb), (IIc), (IId) or (IIe) as described herein) also include a pharmaceutically acceptable salt, solvate, or hydrate thereof.

In certain embodiments, prodrug compounds of the present disclosure (e.g., prodrug compounds that find use in the methods of the present disclosure) include prodrug compounds selected from:

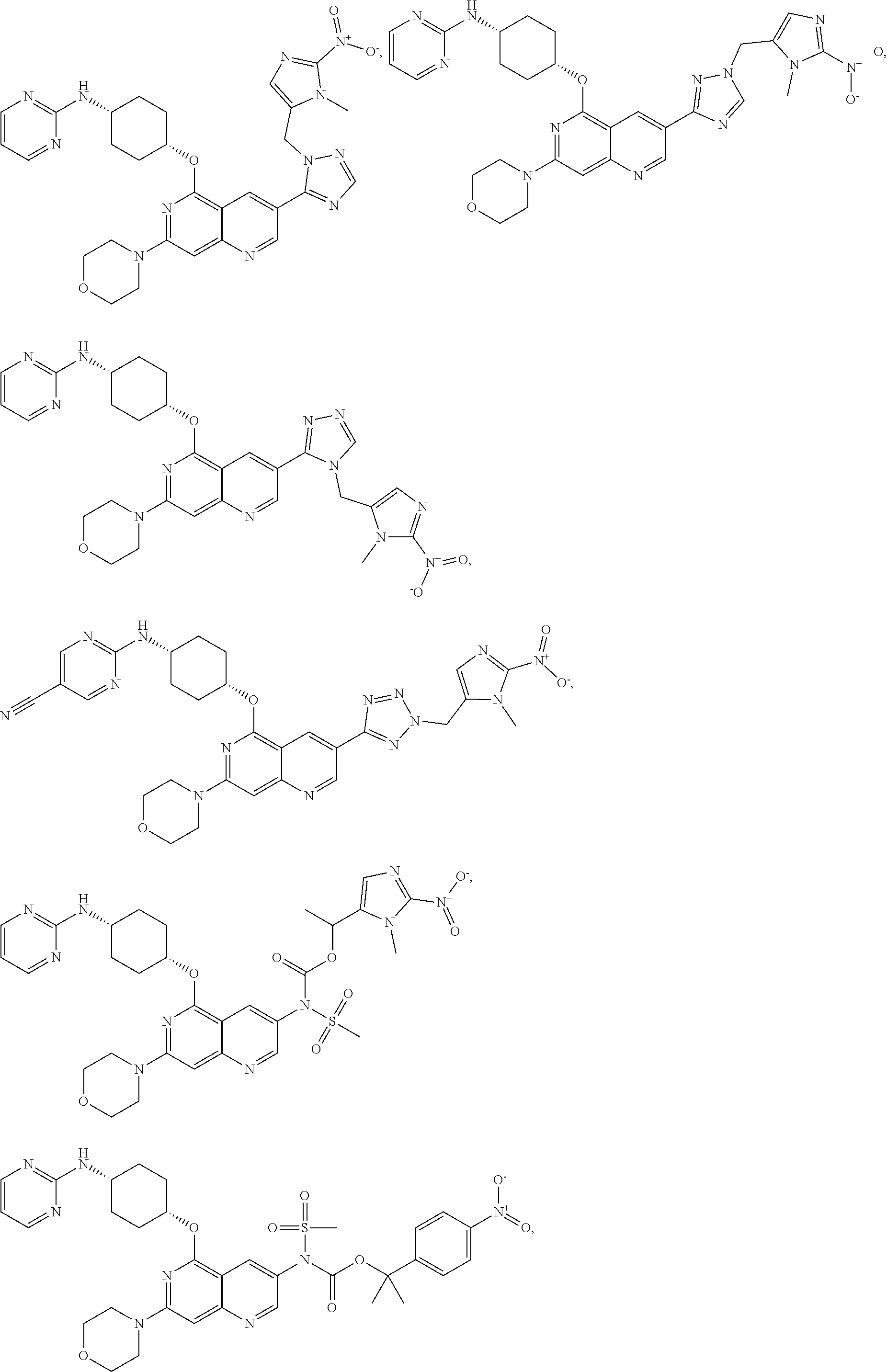

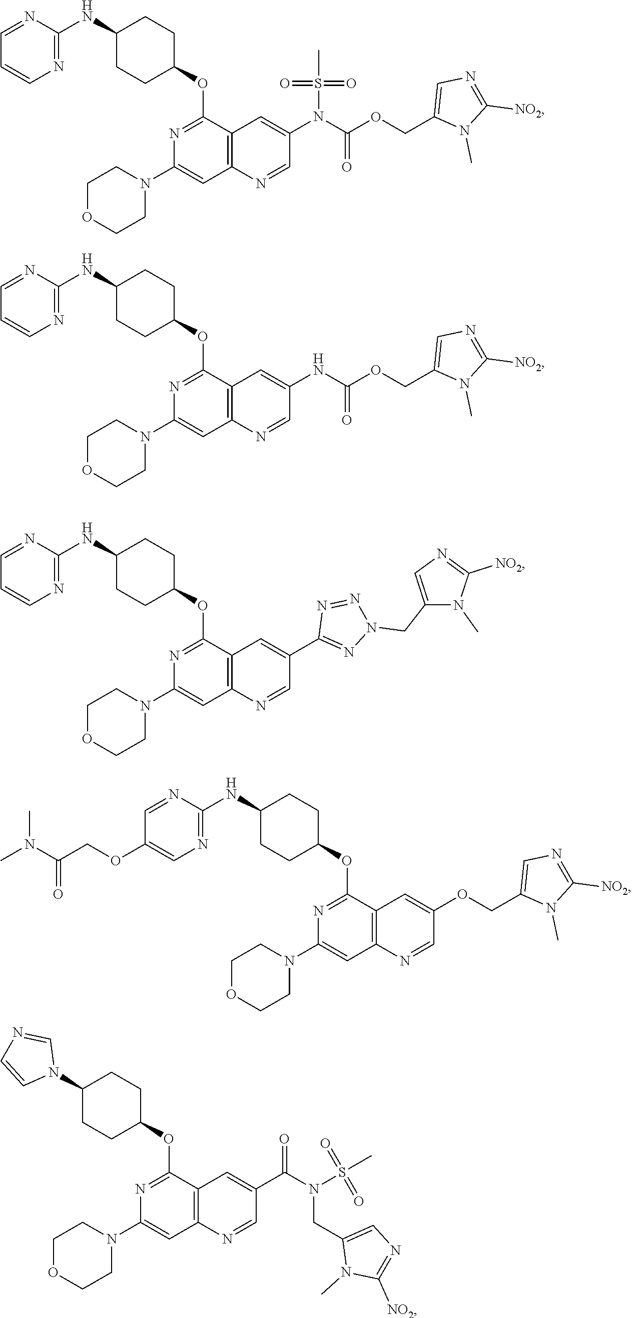

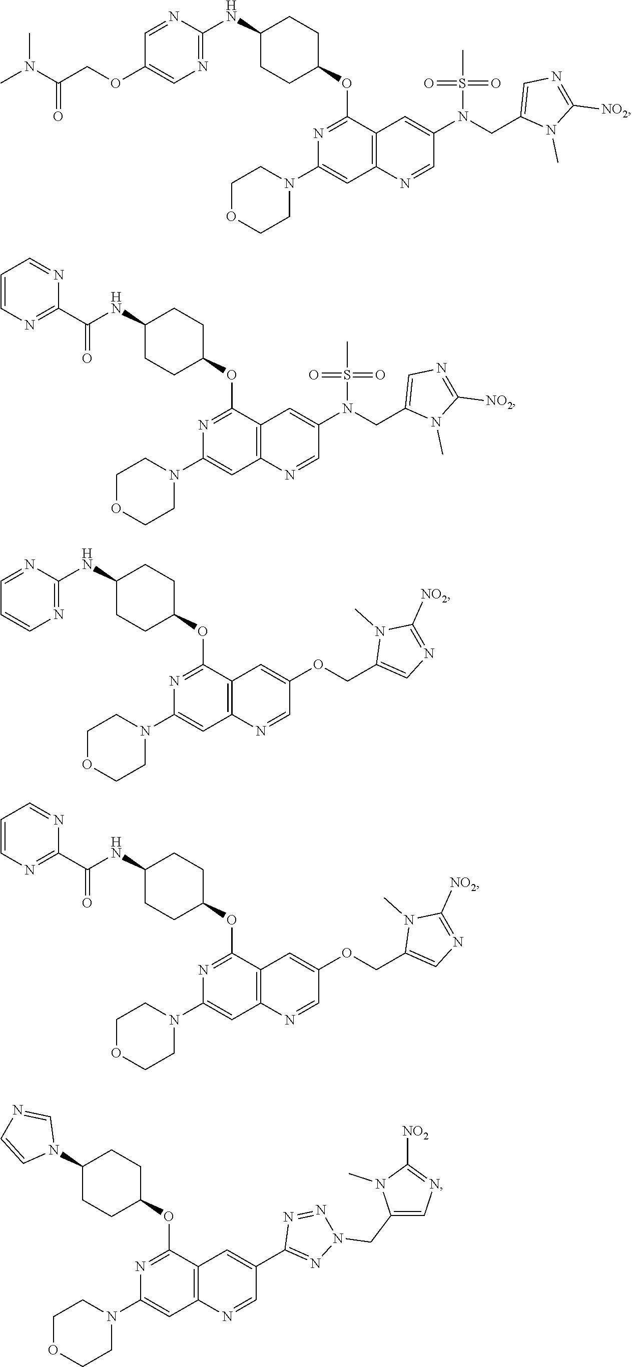

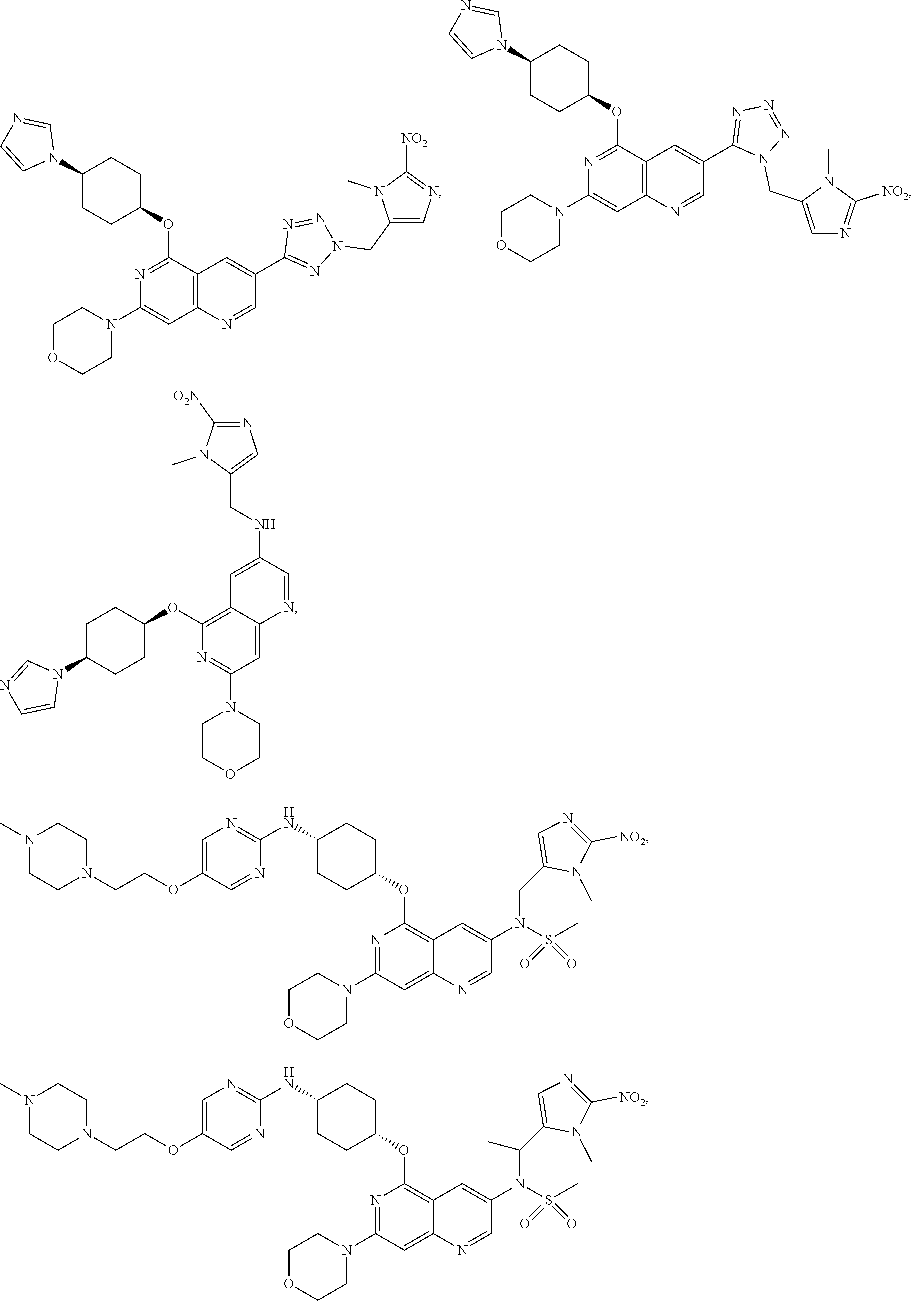 [SMILES: Cn1c(Cn2nnc(-c3cnc4cc(N5CCOCC5)nc(O[C@H]5CC[C@@H](n6ccnc6)CC5)c4c3)n2)cnc1[N+](=O)[O-] ; Cn1c(Cn2nnnc2-c2cnc3cc(N4CCOCC4)nc(O[C@H]4CC[C@@H](n5ccnc5)CC4)c3c2)cnc1[N+](=O)[O-] ; Cn1c(CNc2cnc3cc(N4CCOCC4)nc(O[C@H]4CC[C@@H](n5ccnc5)CC4)c3c2)cnc1[N+](=O)[O-] ; CN1CCN(CCOc2cnc(N[C@H]3CC[C@@H](Oc4nc(N5CCOCC5)cc5ncc(N(Cc6cnc([N+](=O)[O-])n6C)S(C)(=O)=O)cc45)CC3)nc2)CC1 ; CC(c1cnc([N+](=O)[O-])n1C)N(c1cnc2cc(N3CCOCC3)nc(O[C@H]3CC[C@@H](Nc4ncc(OCCN5CCN(C)CC5)cn4)CC3)c2c1)S(C)(=O)=O]

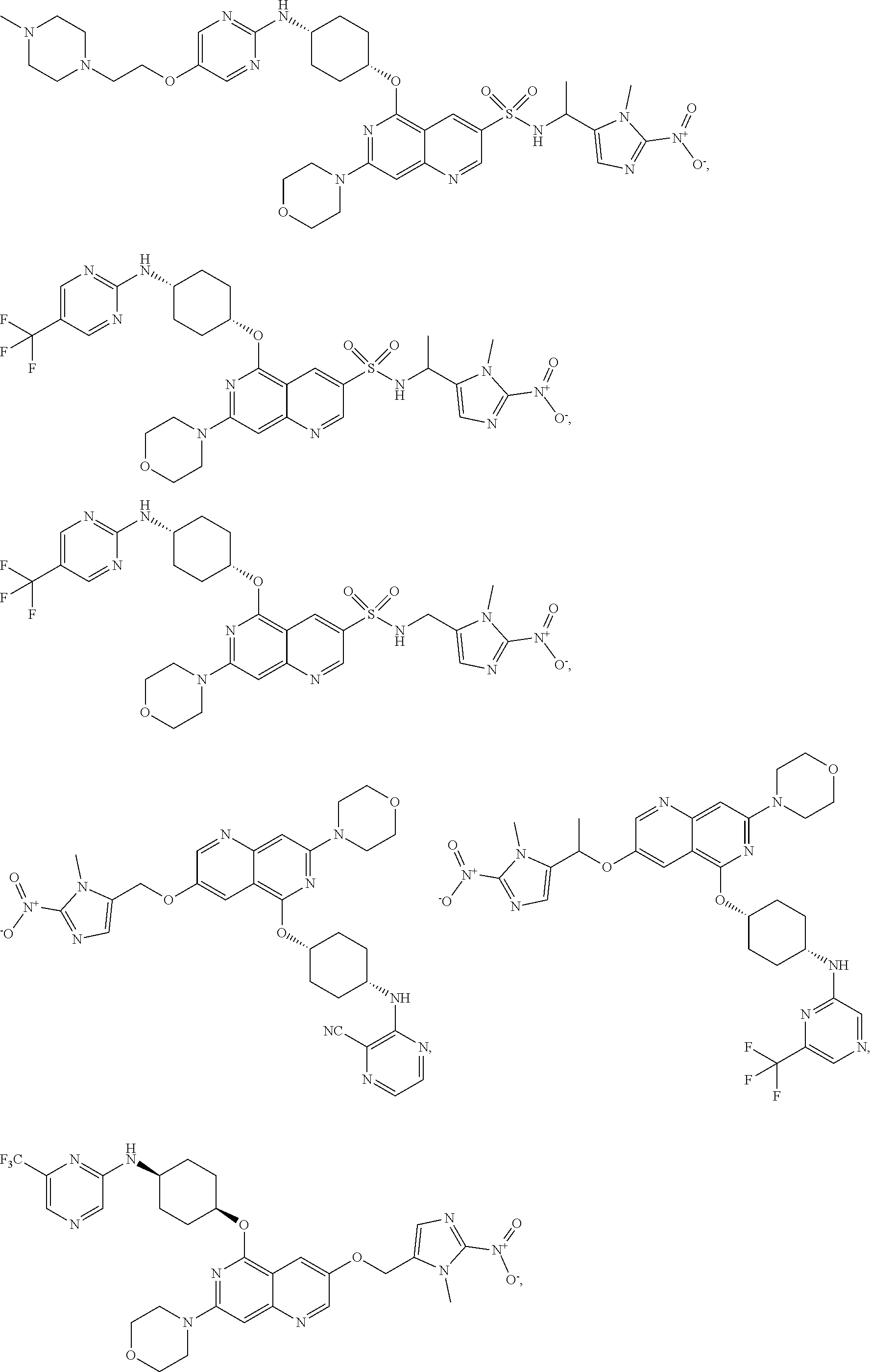

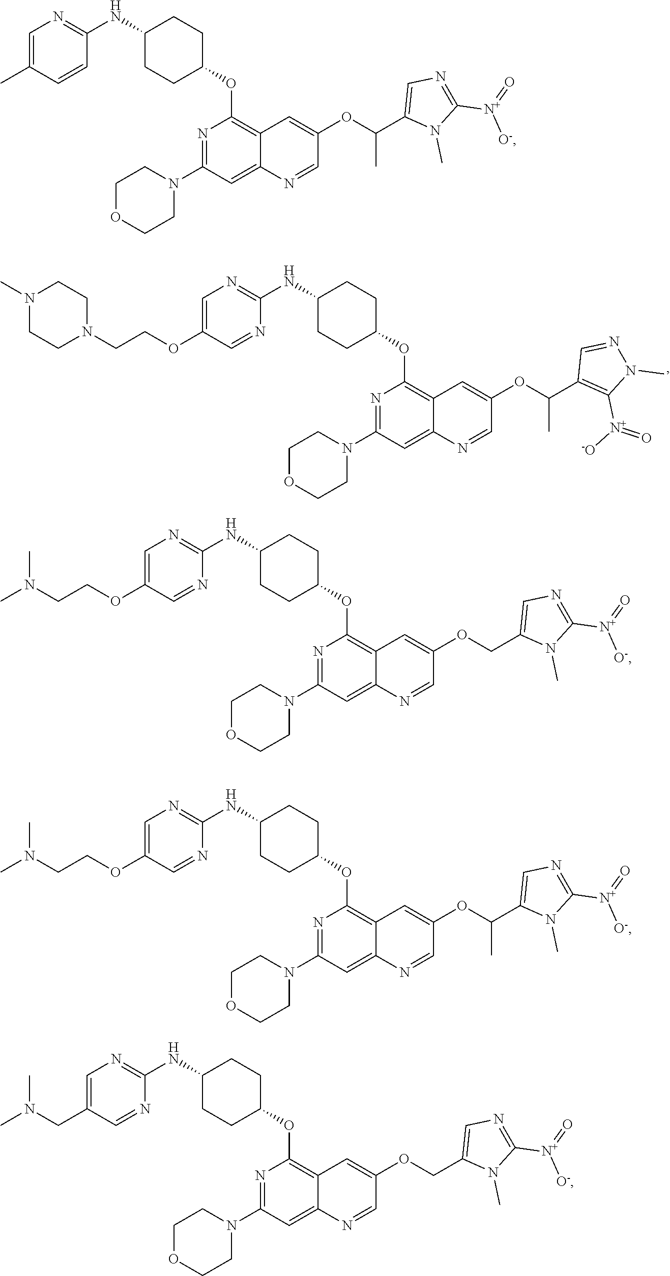

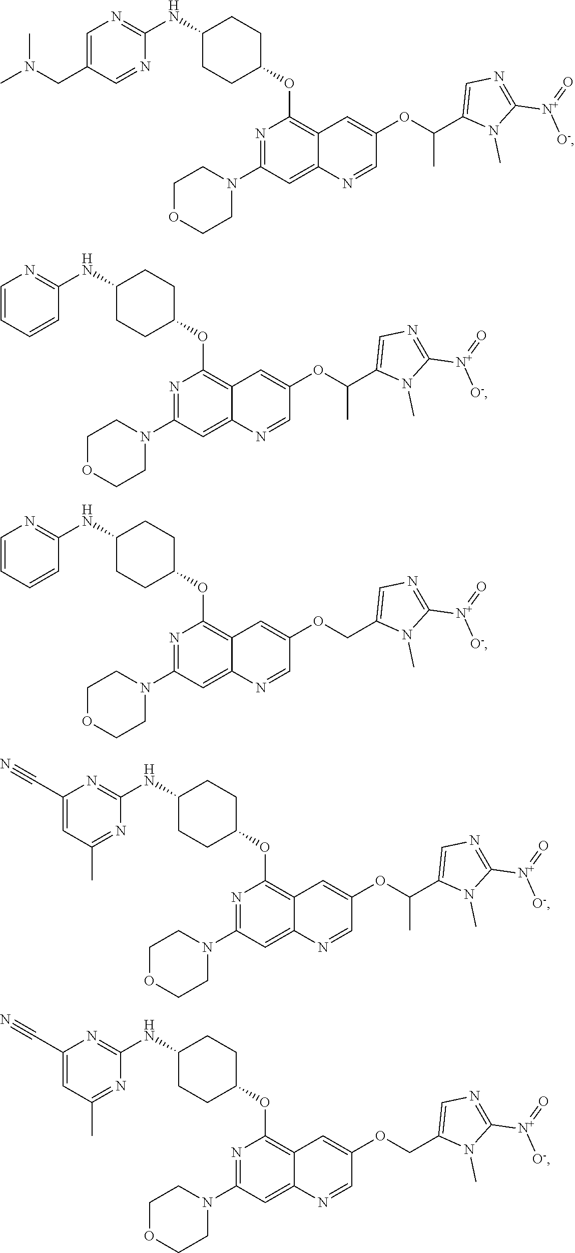

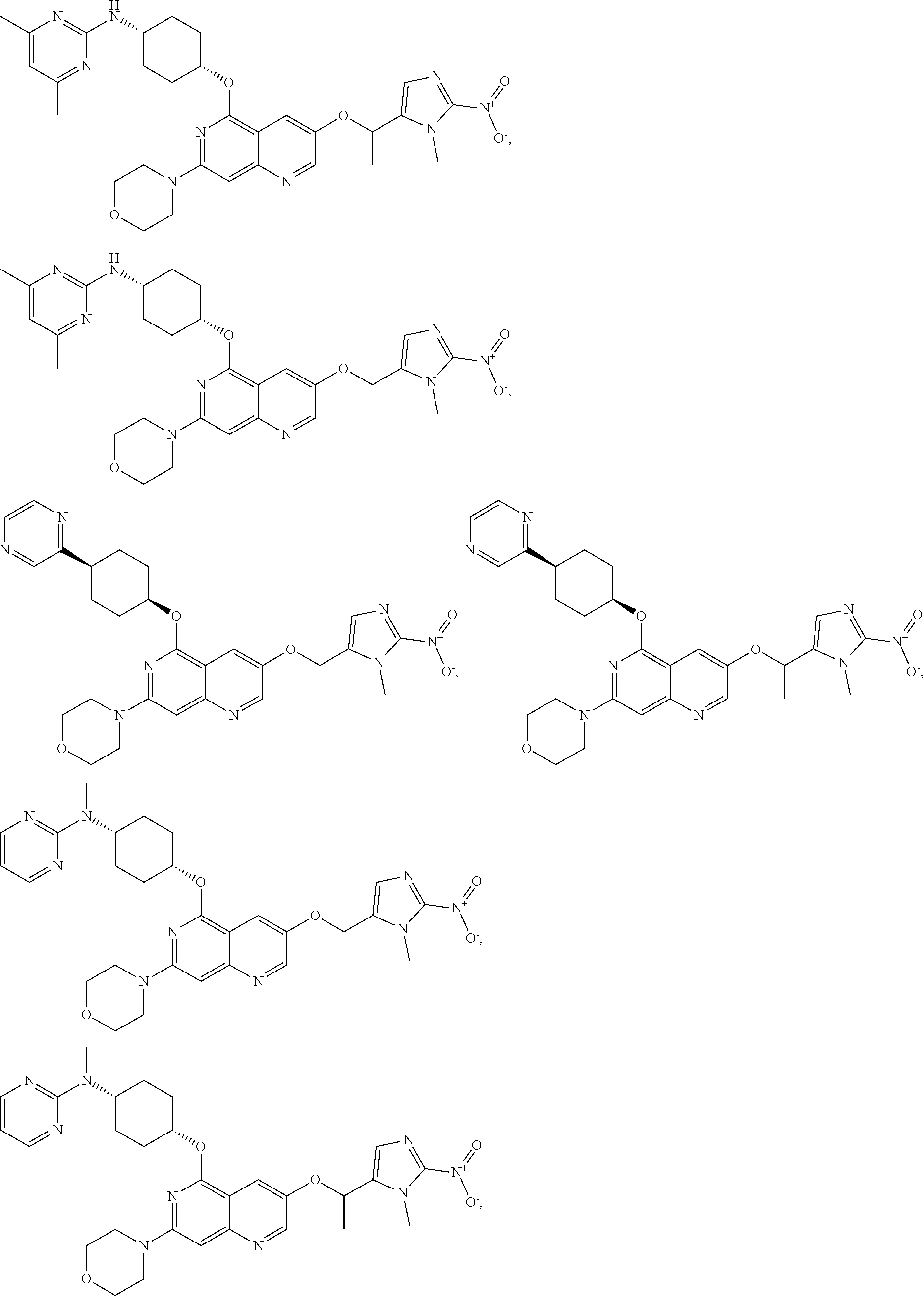

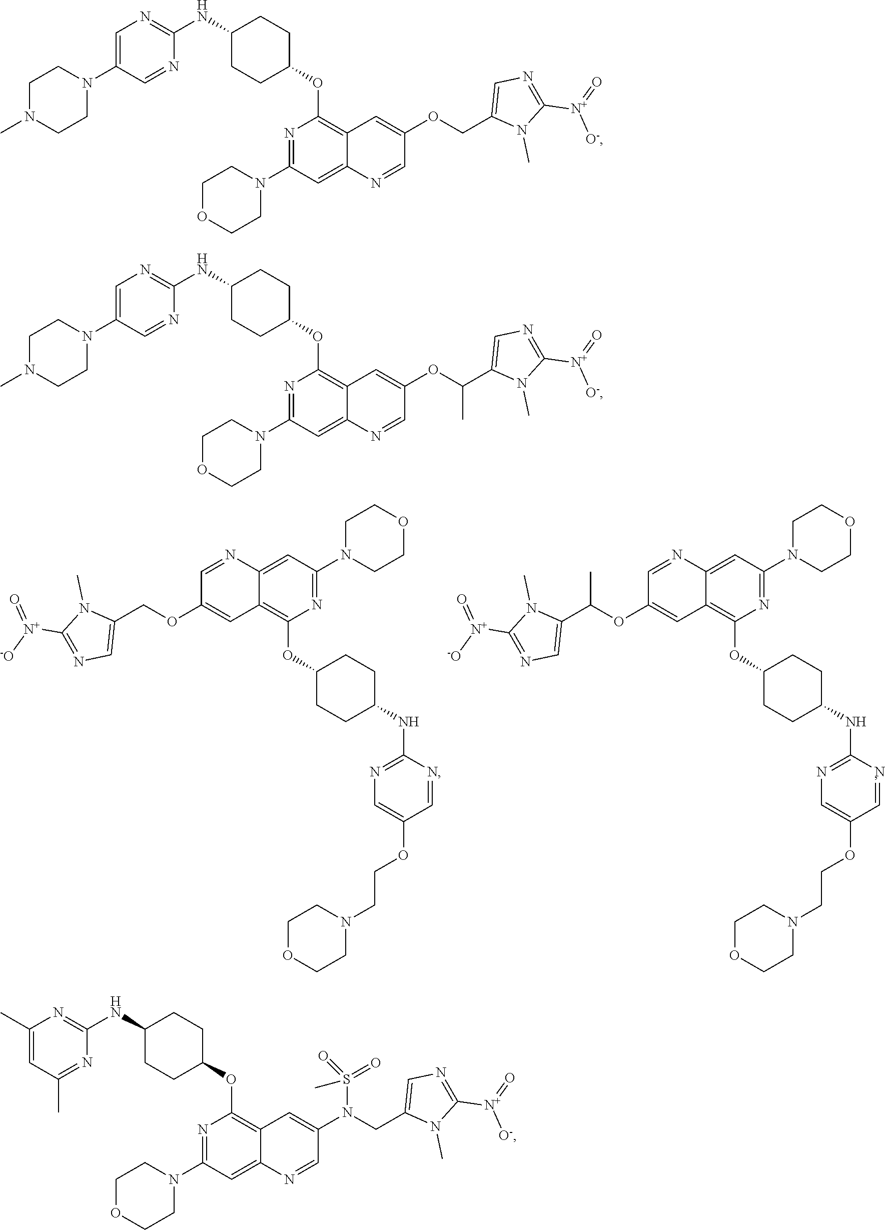

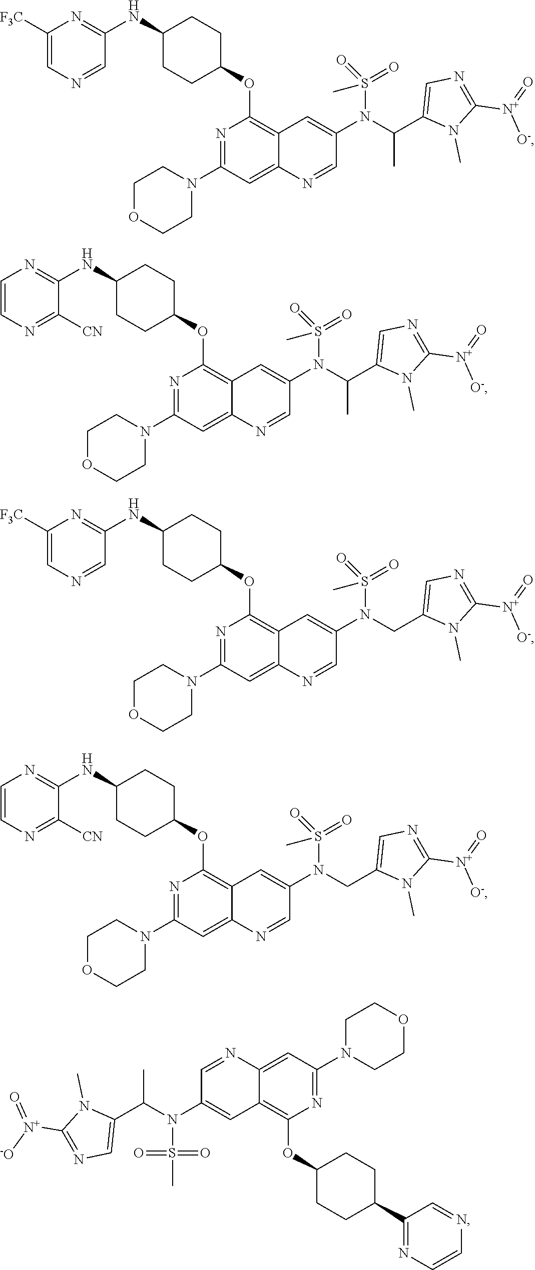

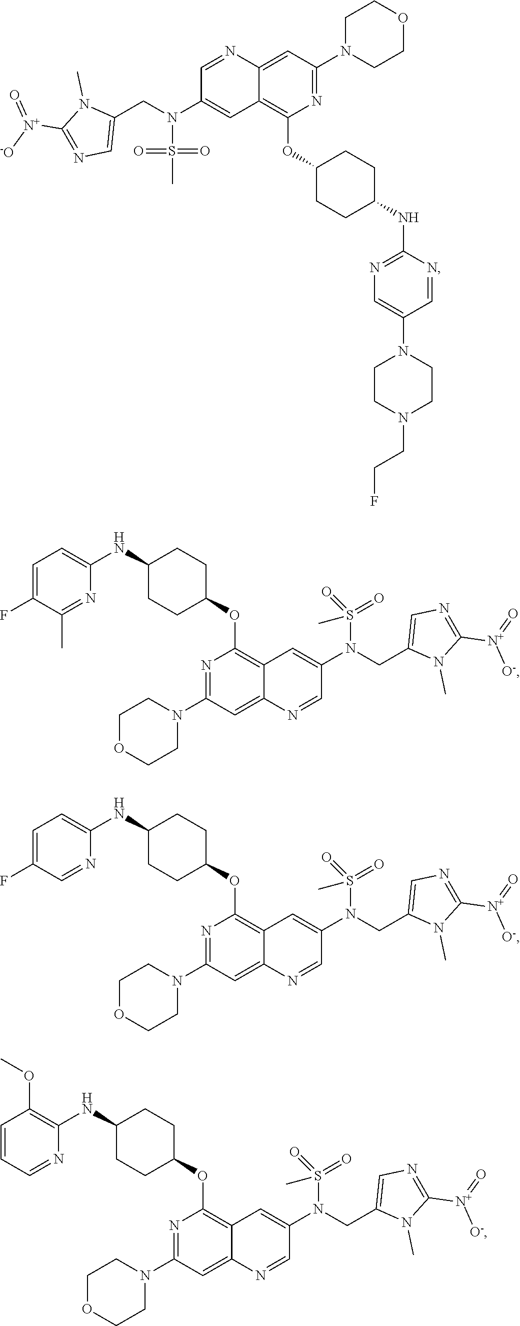

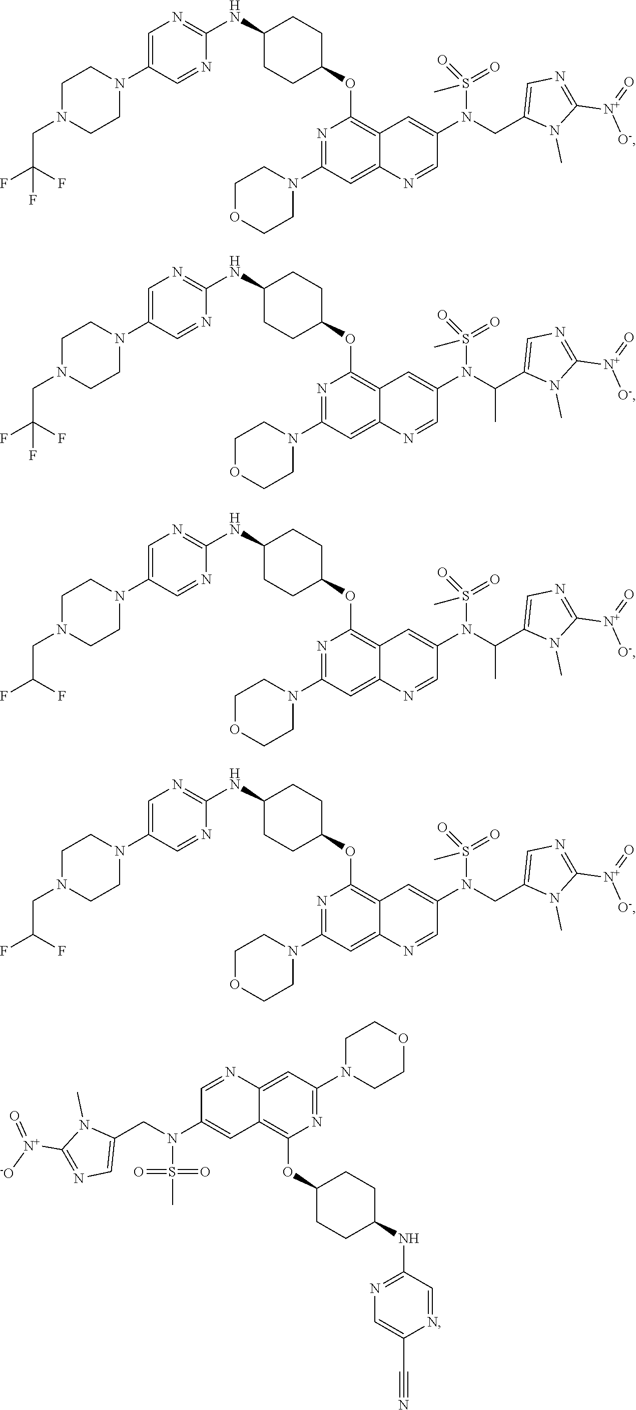

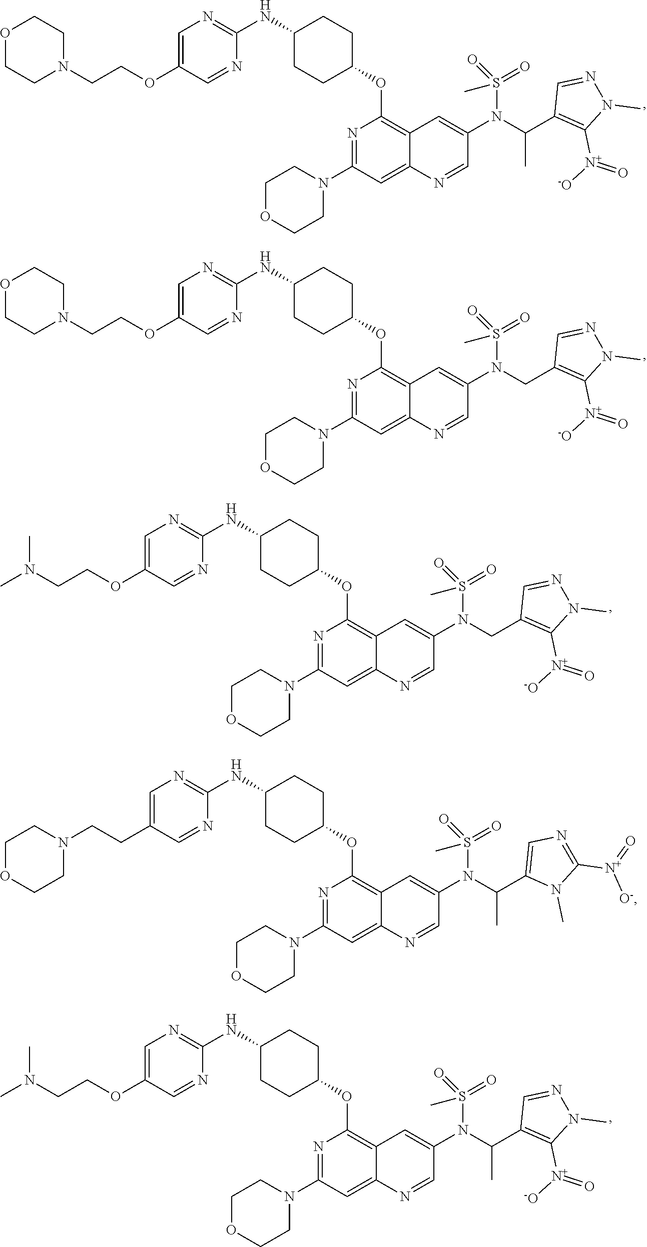

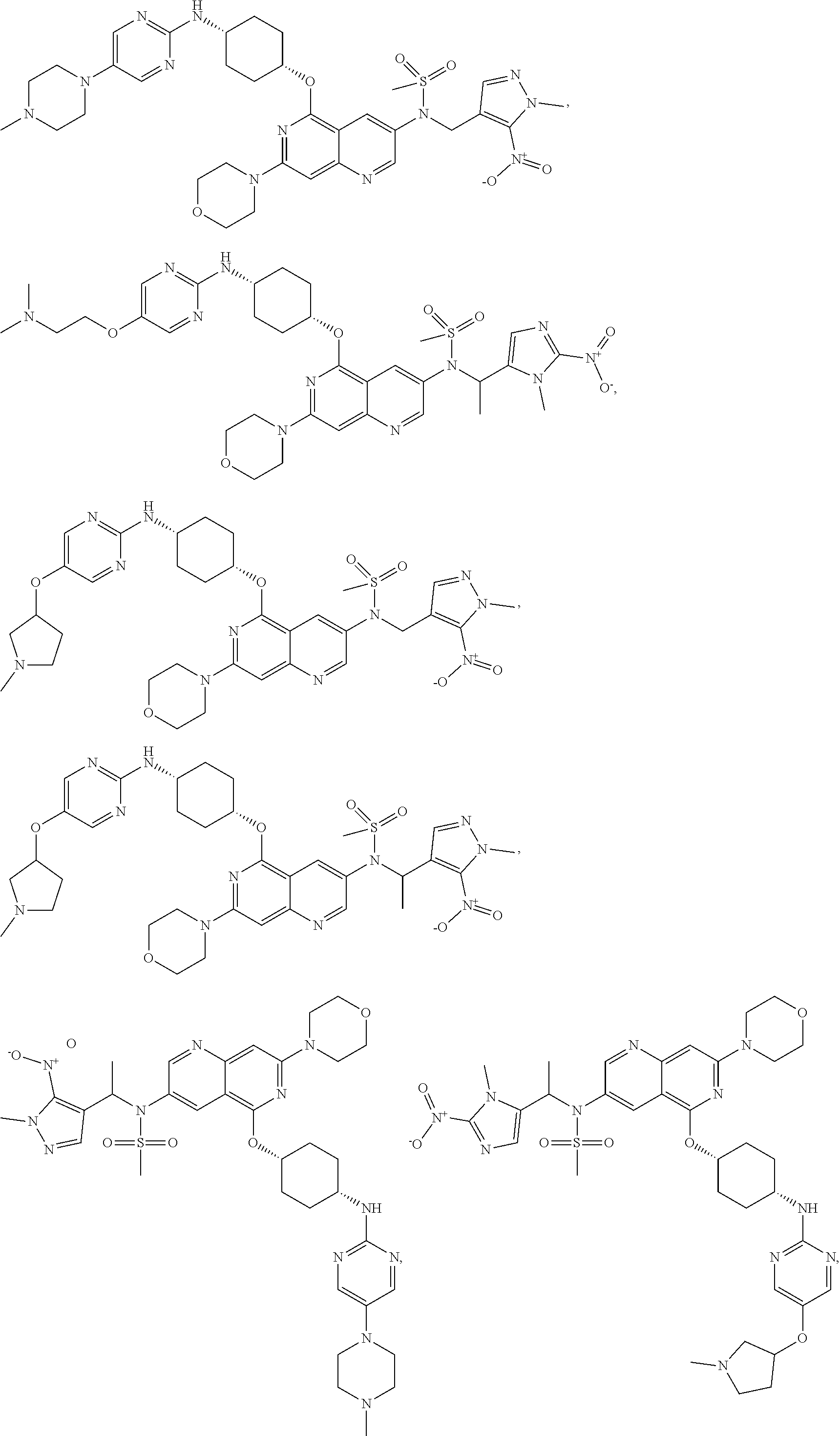

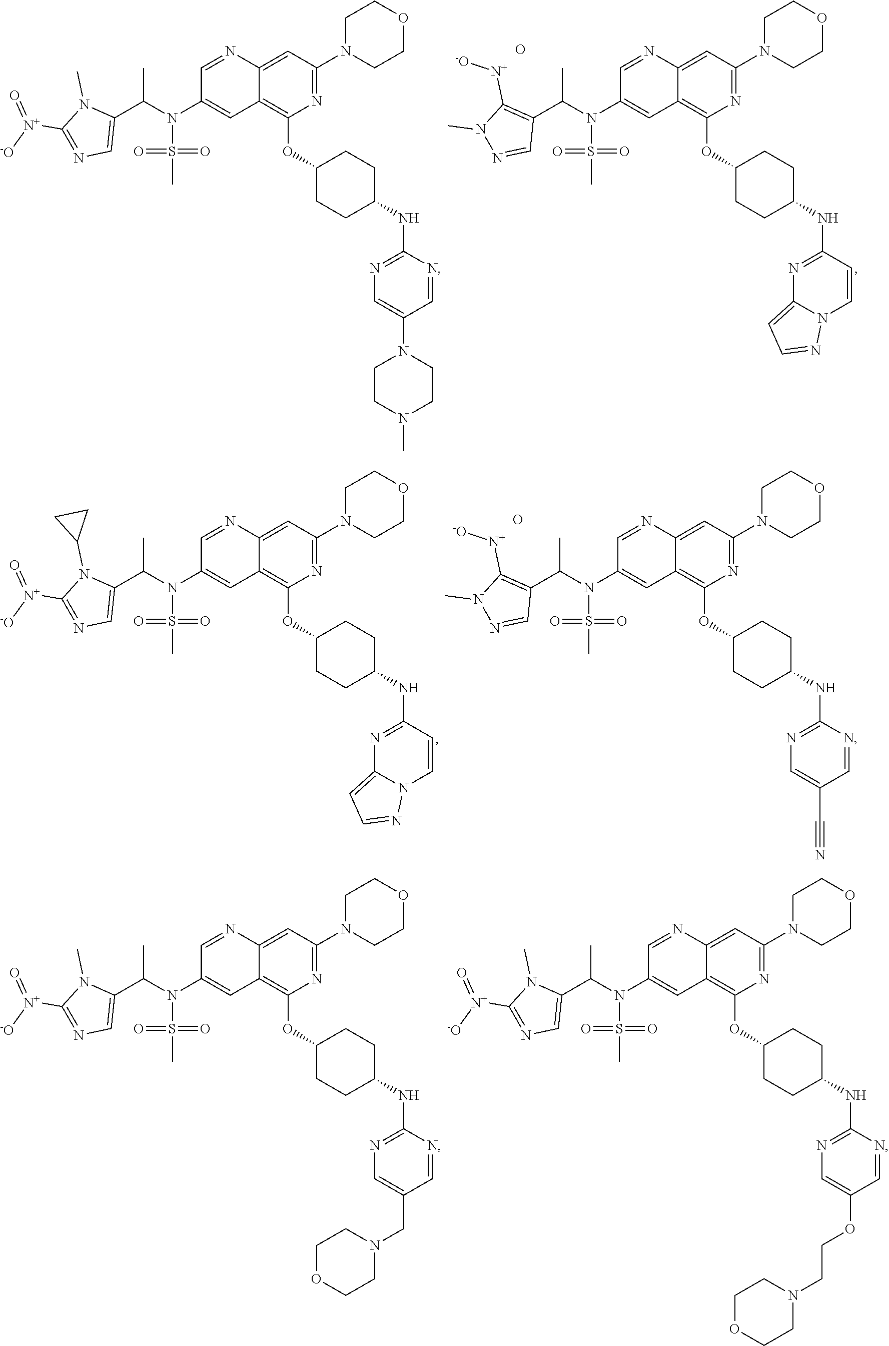

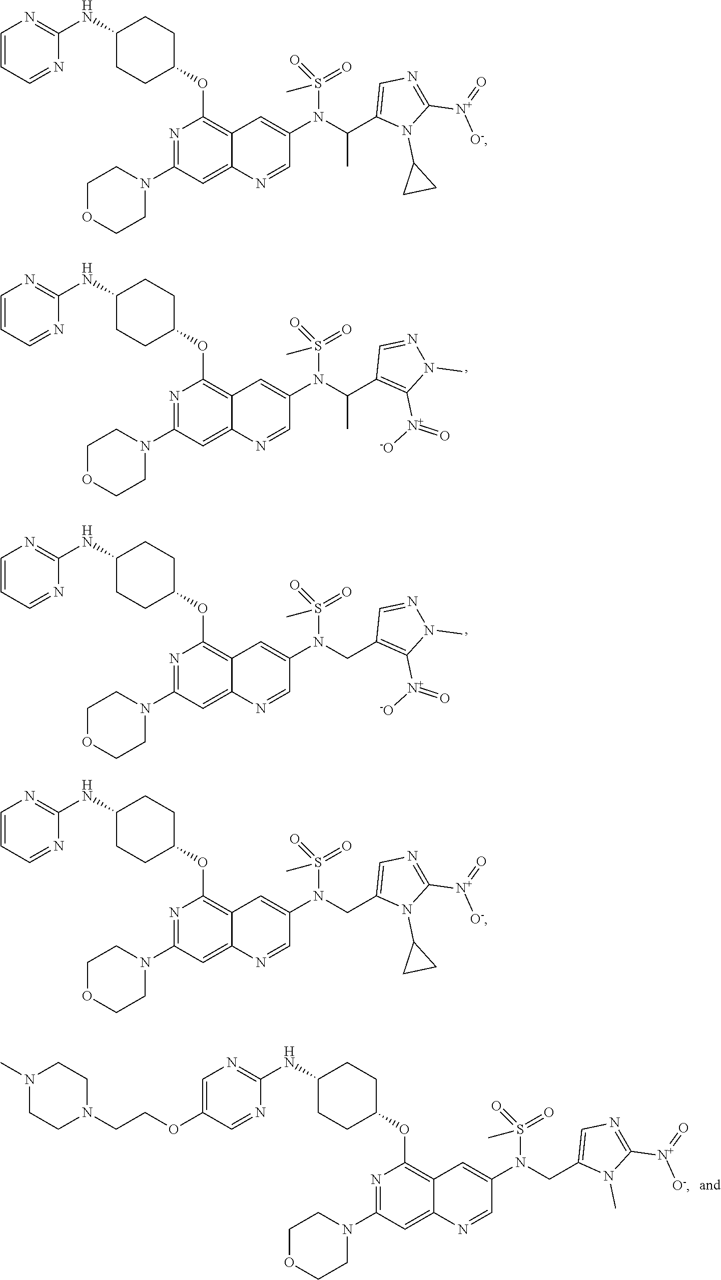

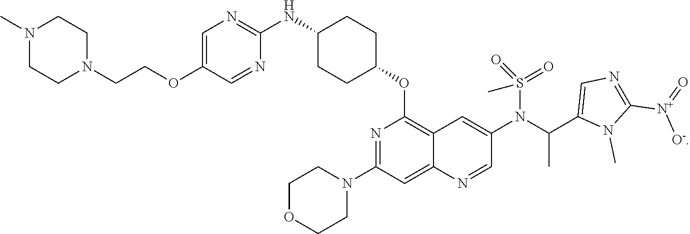

In certain embodiments, prodrug compounds of the present disclosure (e.g., prodrug compounds that find use in the methods of the present disclosure) include prodrug compounds selected from:

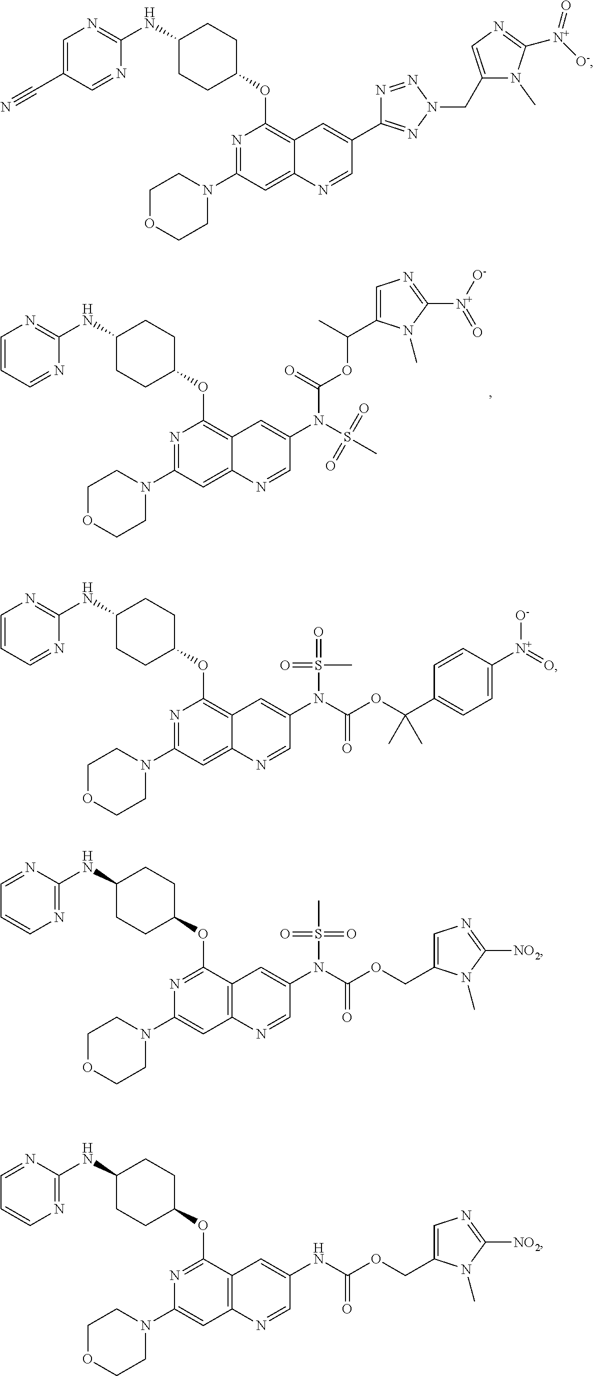

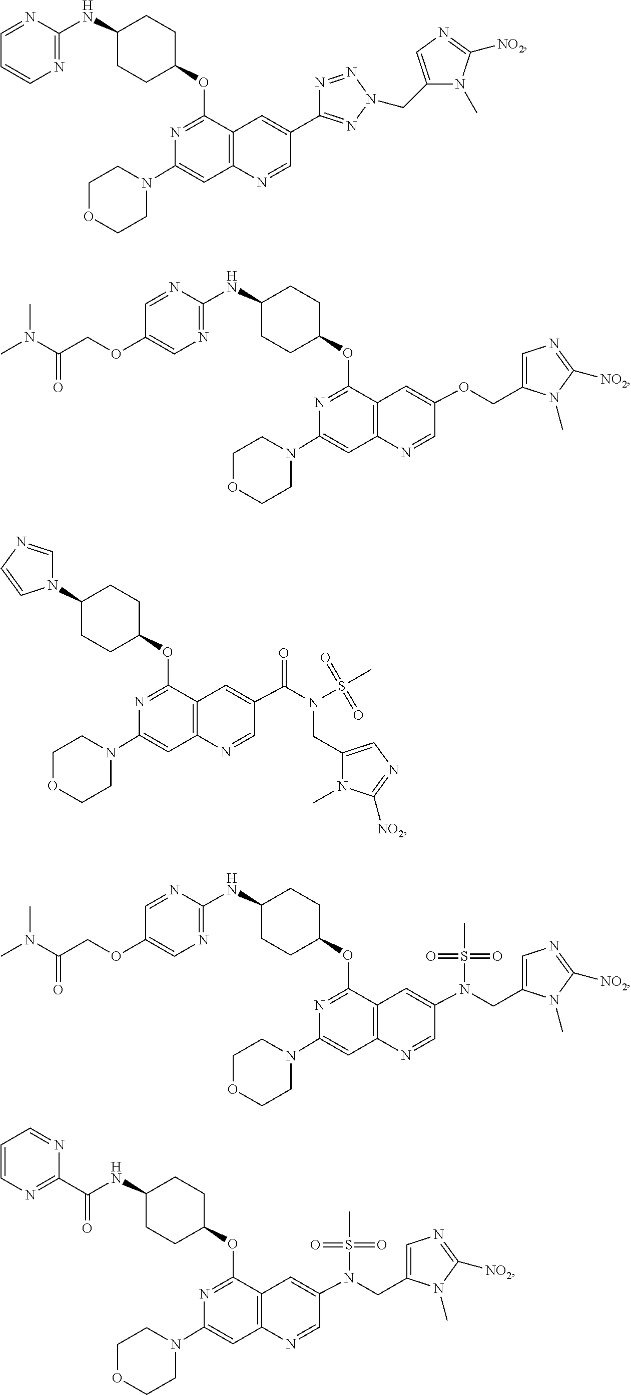 [SMILES: Cn1c(Cn2nnc(-c3cnc4cc(N5CCOCC5)nc(O[C@H]5CC[C@@H](Nc6ncccn6)CC5)c4c3)n2)cnc1[N+](=O)[O-] ; CN(C)C(=O)COc1cnc(N[C@H]2CC[C@@H](Oc3nc(N4CCOCC4)cc4ncc(OCc5cnc([N+](=O)[O-])n5C)cc34)CC2)nc1 ; Cn1c(CN(C(=O)c2cnc3cc(N4CCOCC4)nc(O[C@H]4CC[C@@H](n5ccnc5)CC4)c3c2)S(C)(=O)=O)cnc1[N+](=O)[O-] ; CN(C)C(=O)COc1cnc(N[C@H]2CC[C@@H](Oc3nc(N4CCOCC4)cc4ncc(N(Cc5cnc([N+](=O)[O-])n5C)S(C)(=O)=O)cc34)CC2)nc1 ; Cn1c(CN(c2cnc3cc(N4CCOCC4)nc(O[C@H]4CC[C@@H](NC(=O)c5ncccn5)CC4)c3c2)S(C)(=O)=O)cnc1[N+](=O)[O-]]

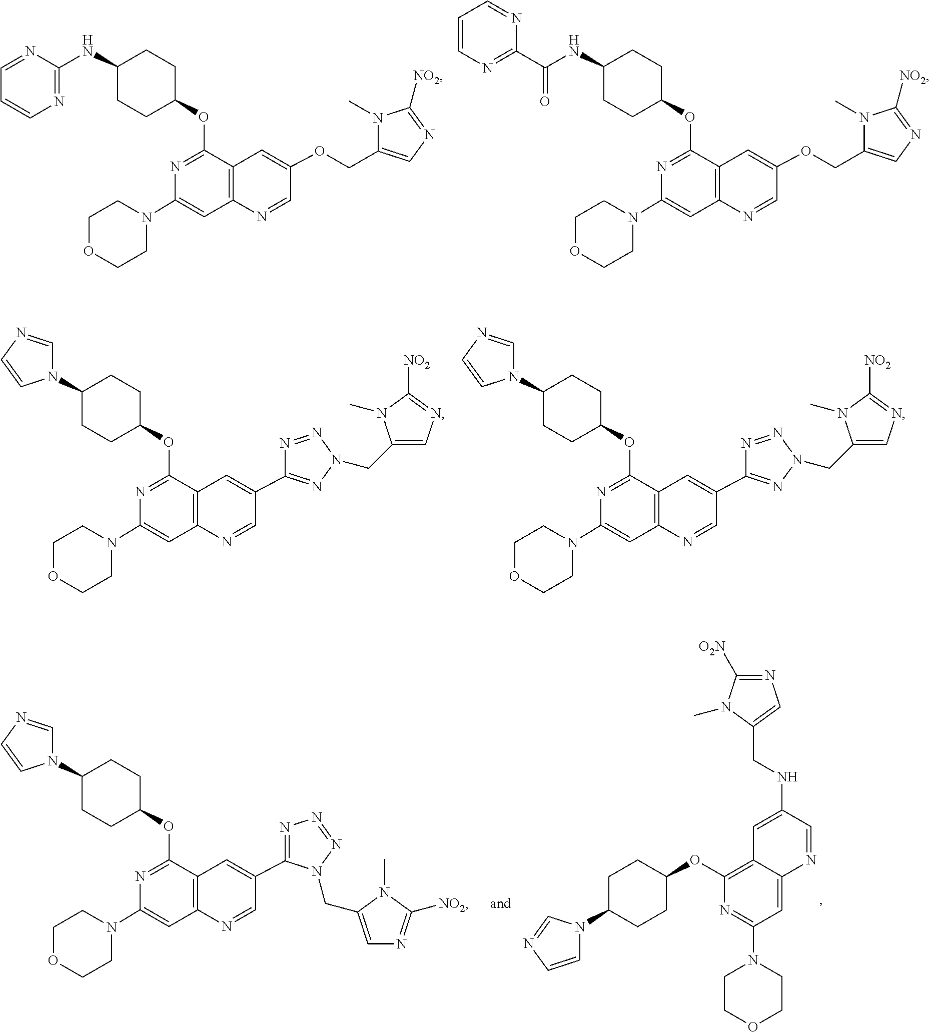

The compounds described herein can be isolated by procedures known to those skilled in the art. The compounds described herein may be obtained, for instance, by a resolution technique or by chromatography techniques (e.g., silica gel chromatography, chiral chromatography, etc.). As used herein, the term “isolated” refers to compounds that are non-naturally occurring and can be obtained or purified from synthetic reaction mixtures. Isolated compounds may find use in the pharmaceutical compositions and methods of treatment described herein.

The compounds described herein also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁵O, ¹⁸O, ¹⁸F, etc. Thus, the disclosed compounds may be enriched in one or more of these isotopes relative to the natural abundance of such isotope. By way of example, deuterium (2H; D) has a natural abundance of about 0.015%. Accordingly, for approximately every 6,500 hydrogen atoms occurring in nature, there is one deuterium atom. Specifically contemplated herein are compounds enriched in deuterium at one or more positions. Thus, deuterium containing compounds of the disclosure have deuterium at one or more positions (as the case may be) in an abundance of greater than 0.015%. In some embodiments, one or more (e.g., 1, 2, 3, 4, 5, 6, 7 or more) hydrogen atoms of a substituent group (e.g., an R-group) of any one of the subject compounds described herein are substituted with a deuterium.

### Methods of Use

In certain embodiments, the compounds and prodrugs of the present disclosure are DNA-PK inhibitors. As such, methods of the present disclosure may include a method of inhibiting DNA-PK activity by contacting DNA-PK with a compound or prodrug of the present disclosure. The contacting may be sufficient to inhibit the activity of DNA-PK as compared to DNA-PK in the absence of a compound or prodrug of the present disclosure.

### Methods of Treatment

The compounds and prodrugs of the present disclosure find use in treatment of a condition or disease in a subject that is amenable to treatment by administration of the compound. Thus, in some embodiments, provided are methods that include administering to a subject a therapeutically effective amount of any of the compounds or prodrugs of the present disclosure (including prodrugs thereof). In certain aspects, provided are methods of delivering a compound or prodrug to a subject, the method including administering to the subject an effective amount of a compound or prodrug of the present disclosure. In certain instances, the administering is effective to provide a therapeutically effective amount of the compound or prodrug to the subject.

The subject to be treated can be one that is in need of therapy, where the subject to be treated is one amenable to treatment using the compounds disclosed herein (including prodrugs thereof). Accordingly, a variety of subjects may be amenable to treatment using the compounds or prodrugs disclosed herein. Generally, such subjects are “mammals”, with humans being of interest. Other subjects can include companion animals or domestic pets (e.g., canine and feline), livestock (e.g., cows, pigs, goats, horses, and the like), rodents (e.g., mice, guinea pigs, and rats, e.g., as in animal models of disease), as well as non-human primates (e.g., chimpanzees, and monkeys). In some instances, the mammal is selected from a companion animal and livestock. In some instances, the mammal is feline. In some instances, the mammal is a human.

The present disclosure provides methods that include delivering a compound or prodrug of the present disclosure to an individual having a disease, such as methods that include administering to the subject a therapeutically effective amount of a compound of the present disclosure (including prodrugs thereof). The methods are useful for treating a wide variety of conditions and/or symptoms associated with a disease. In the context of disease, the term “treating” includes one or more (e.g., each) of: reducing the severity of one or more symptoms, inhibiting the progression, reducing the duration of one or more symptoms, and ameliorating one or more symptoms associated with the disease.

The administering can be done any convenient way. Generally, administration is, for example, oral, buccal, parenteral (e.g., intravenous, intraarterial, subcutaneous), intraperitoneal (i.e., into the body cavity), topically, e.g., by inhalation or aeration (i.e., through the mouth or nose), or rectally systemic (i.e., affecting the entire body). For example, the administration may be systemic, e.g., orally (via injection of tablet, pill or liquid) or intravenously (by injection or via a drip, for example). In other embodiments, the administering can be done by pulmonary administration, e.g., using an inhaler or nebulizer. Compounds of the present disclosure or composition comprising the compounds may be administered in dosage unit formulations containing conventional non-toxic pharmaceutically acceptable carriers, adjuvants, and vehicles as desired. The term “topically” may include injection, insertion, implantation, topical application, or parenteral application.

In certain embodiments, the compounds and prodrugs of the present disclosure find use in methods of treating cancer in a subject. Thus, in some embodiments, provided are methods that include administering to a subject a therapeutically effective amount of any of the compounds of the present disclosure (including prodrugs thereof). In certain instances, the administering is effective to provide a therapeutically effective amount of the compound to the subject to treat a cancer in the subject. In certain embodiments, the cancer may be selected from acute lymphoblastic leukemia, acute lymphocytic leukemia, acute megakaryocytic leukemia, acute myelogenous leukemia, Acute myeloid leukemia, acute nonlymphocytic leukemia, adenocarcinoma of the lung and squamous carcinoma of the lung, Adrenocortical carcinoma, AIDS-related cancers, AIDS-related lymphoma, anal cancer, anal carcinoma, anaplastic astrocytoma, appendix cancer, arrhenoblastomas, astrocytic brain tumors, astrocytoma, B cell lymphomas. basal cell carcinoma (basal cell epithelioma), bile duct cancer, biliary cancer, bladder cancer (e.g., urothelial bladder cancer), blood cell malignancies, bone cancers, bone sarcoma, bone tumor, bowel cancer, brain cancer (e.g., astrocytoma), brain tumor, brainstem glioma, breast cancer, bronchial carcinoids, buccal cancer, Burkitt's lymphoma, cancer of the mouth, cancer of the peritoneum, carcinoid tumor, castration-resistant prostate cancer, central nervous system cancer, cerebellar astrocytoma, cervical cancer, cervical carcinoma, cholangiocarcinoma, chondrosarcoma, choriocarcinoma, chronic lymphocyte leukemia chronic lymphocytic leukemia, chronic myelogenous leukemias, chronic neutrophilic leukemia, colon cancer, colorectal cancer, colorectal neoplasia, cutaneous T cell lymphoma, diffuse large B-cell lymphoma, effusion lymphomas, endometrial or uterine carcinoma, ependymoma, epithelial ovarian cancer, erythroleukemia, esophageal cancer, esophageal carcinomas, esophageal squamous cell carcinoma, Ewing's sarcoma, eye cancer, fallopian tube cancer, fibrosarcomas, follicular lymphoma.gall bladder cancer, gastric adenocarcinoma, gastric or stomach cancer includinggastrointestinal cancer, gastrointestinal stromal tumor, glioblastoma multiforme (GBM), glioblastoma, gliomas, gliosarcoma, hairy-cell leukemia, head and neck cancer, head and neck squamous cell carcinoma (HNSCC), hemangiopericytoma, hematologic malignancies, hepatic carcinoma, hepatoma, Hodgkin lymphoma, hormone-refractory prostate cancer, immunoblastic large cell leukemia, intraocular (eye), intraocular melanoma, Kaposi's sarcoma, kidney or renal cancer (e.g., renal cell carcinoma, nephroblastoma or Wilms' tumor), large cell neuroendocrine cancer, larynx cancer, laryngeal carcinomas, leiomyosarcomas, leukemia, Liposarcoma, liver cancer (e.g., hepatocellular carcinoma (HCC)), lung cancer including small-cell lung cancer, lymphoblastic T cell leukemia, lymphoblastic T cell lymphoma, lymphoid cancer, malignant pleural effusion, malignant pleural mesothelioma head and neck cancer, mantle cell leukemia, medulloblastoma, melanoma, Merkel cell carcinoma, mesothelioma, metastatic breast cancer, metastatic colorectal cancer, metastatic liver lesions, metastatic melanoma, metastatic renal cell carcinoma, metastatic renal clear cell carcinoma, multiple myeloma and acute hematologic malignancies, muscle-invasive bladder cancer, nasopharyngeal cancer, nasopharyngeal carcinoma, neuroblastomas, neuroendocrine cancer, neuroendocrine prostate cancer, neuroendocrine tumors (NETS), non-Hodgkin's lymphoma, non-muscle invasive bladder cancer, non-small cell cancers, non-small cell lung cancer (“NSCLC”), oligodendroglioma, oral carcinoma, osteogenic sarcoma, osteosarcoma, ovarian cancer (e.g., high grade serous ovarian carcinoma), ovarian carcinoma, pancreatic cancer (e.g., pancreatic ductal adenocarcinoma), papillary carcinoma, parathyroid cancer, parotid gland cancer, penile cancer, penile carcinoma, peritoneal cancer, plasmacytoma, promyelocytic leukemia, prostate cancer, rectal cancer, recurrent glioblastoma multiforme (GBM), recurrent head and neck cancer squamous cell carcinoma, relapsed or refractory small-cell lung cancer, renal cancer, renal cell carcinoma, retinoblastoma, rhabdomyosarcoma, salivary gland cancer, salivary gland carcinoma, sarcomas, Schwannoma, skin cancer, skin carcinomas, small cell bladder cancer, small cell lung carcinoma, small intestine cancer, soft tissue sarcoma, squamous carcinoma of the lung, squamous cell cancer (e.g., epithelial squamous cell cancer), squamous cell carcinoma. squamous non-small cell lung cancer, T cell lymphocytic leukemia, testicular (germ cell tumor) cancer, throat cancer, thymic lymphoma lung cancer, thymoma, thyroid cancer, transitional cell cancer, treatment-resistant melanoma, ureter or renal pelvis cancer, urethral cancer, urinary tract carcinomas, urothelial cancer, uterine cancer and solid tumors in the ovarian follicle, uterine endometrial cancers, uterine sarcoma, vaginal cancer, vulval cancer, and Waldenstrom macroglobulinemia. In certain embodiments, the types of cancers that can be treated using the compounds, prodrugs and methods of the present disclosure include a solid cancer or solid tumor. For example, the cancer may be selected from: lung cancer, rectal cancer, colon cancer, liver cancer, bladder cancer, breast cancer, biliary cancer, prostate cancer, ovarian cancer, stomach cancer, bowel cancer, skin cancer, pancreatic cancer, brain cancer, cervix cancer, anal cancer, and head and neck cancer, and the like. In some embodiments the cancer may be head and neck cancer. In some embodiments, the cancer may be head and neck squamous cell carcinoma (HNSCC). In some embodiments, the cancer may be an ATM gene mutation-associated cancer. In certain embodiments, the cancer may be selected from bladder cancer, brain cancer, breast cancer, central nervous system cancer, larynx cancer, leukemia, liver cancer, lung cancer, lymphoma, ovarian cancer, pancreatic cancer, parotid gland cancer, prostate cancer, skin cancer, and stomach cancer.

In certain embodiments, the method of treating cancer in a subject further includes treating the subject with radiotherapy and/or a DNA damaging chemotherapeutic agent. Compounds of the present disclosure are DNA-PK inhibitors and are expected to enhance the effectiveness of cancer therapies that induce DNA damage in cancer cells, particularly hypoxic cancer cells. Accordingly, compounds of the present disclosure can be used in methods for treating cancer in a subject, where the compound or the prodrug thereof sensitizes cancer cells to radiotherapy and/or a DNA damaging chemotherapeutic agent.

In certain embodiments, methods of treating cancer in a subject include administering a compound or prodrug of the present disclosure together with a DNA damaging chemotherapeutic agent in the treatment of a cancer in the subject. In some cases, the compound or prodrug of the present disclosure can be administered in combination with a DNA damaging chemotherapeutic agent. In some instances, the method includes administering a compound or prodrug of the present disclosure simultaneously, sequentially or separately with a DNA damaging chemotherapeutic agent. For example, the compounds and prodrugs of the present disclosure may be used in combination with an anti-tumor agent, particularly anti-tumor agents that induce DNA damage. The compounds and prodrugs of the present disclosure may therefore be used in combination with one or more additional anti-tumor agents to enable a lower dose of the additional anti-tumor agent to be administered while maintaining or enhancing the anticancer effect of the additional anti-tumor agent. Accordingly, the compounds and prodrugs of the present disclosure may increase the therapeutic window and reduce undesirable side effects associated with the additional anti-tumor agent.

DNA damaging chemotherapeutic agents that may be used together with the compounds and prodrugs of the present disclosure include chemotherapeutic agents that induce DNA cross-links or function as topoisomerase inhibitors, inducing the generation of double strand-breaks in DNA. Examples of DNA damaging chemotherapeutic agents include, but are not limited to, platinum anticancer agents (e.g. cisplatin, carboplatin, oxaliplatin or picoplatin); anthracyclines (e.g. doxorubicin or daunorubicin); antifolates (e.g. methotrexate or pemetrexed); 5-fluorouracil; etoposide; gemcitabine; capecitabine; 6-mercaptopurine; 8-azaguanine; fludarabine; cladribine; vinorelbine; cyclophosphamide; taxoids (e.g. taxol, taxotere or paclitaxel), DNA-alkylating agents (e.g. nitrosoureas such as carmustine, lomustine or semustine); triazenes (e.g. dacarbazine or temozolomide); mitomycin C; and streptozotocin; and the like, and combinations thereof. In some instances, the method includes administering a compound or prodrug of the present disclosure simultaneously, sequentially or separately with a DNA damaging chemotherapeutic agent.

Other anti-tumor agents may include, for example, one or more of the following categories of anti-tumor agents: (i) antiproliferative/antineoplastic drugs and combinations thereof, such as alkylating agents (for example a platinum drug (e.g. cis-platin, oxaliplatin or carboplatin), cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine); antimetabolites (for example gemcitabine and antifolates such as fluoropyrimidines like 5-fluorouracil and tegafur, raltitrexed, methotrexate, pemetrexed, cytosine arabinoside, floxuridine, cytarabine, 6-mercaptopurine, 6-thioguanine, fludarabine phosphate, pentostatine, and gemcitabine and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, irinotecan, mitoxantrone and camptothecin); bleomcin, dactinomycin, daunorubicin, doxorubicin, epirubicin, idarubicin, ara-C, paclitaxel (Taxol™), nabpaclitaxel, docetaxel, mithramycin, deoxyco-formycin, mitomycin-C, L-asparaginase, interferons (especially IFN-alpha), etoposide, teniposide, DNA-demethylating agents, (for example, azacitidine or decitabine); and histone de-acetylase (HDAC) inhibitors (for example vorinostat, MS-275, panobinostat, romidepsin, valproic acid, mocetinostat (MGCD0103) and pracinostat SB939); (ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride; and navelbene, CPT-II, anastrazole, letrazole, capecitabine, reloxafme, cyclophosphamide, ifosamide, and droloxafine; (iii) anti-invasion agents, for example dasatinib and bosutinib (SKI-606), and metalloproteinase inhibitors, inhibitors of urokinase plasminogen activator receptor function or antibodies to Heparanase; (iv) inhibitors of growth factor function: for example such inhibitors include growth factor antibodies and growth factor receptor antibodies, for example the anti-erbB2 antibody trastuzumab [Herceptin™], the anti-EGFR antibody panitumumab, the anti-erbB1 antibody cetuximab, tyrosine kinase inhibitors, for example inhibitors of the epidermal growth factor family (for example EGFR family tyrosine kinase inhibitors such as gefitinib, erlotinib, 6-acrylamido-N-(3-chloro-4-fluorophenyl)-7-(3-morpholinopropoxy)-quinazolin-4-amine (CI-1033), erbB2 tyrosine kinase inhibitors such as lapatinib) and antibodies to costimulatory molecules such as CTLA-4, 4-IBB and PD-1, or antibodies to cytokines (IL-IO, TGF-beta); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signaling inhibitors such as farnesyl transferase inhibitors, for example sorafenib, tipifarnib and lonafarnib), inhibitors of cell signaling through MEK and/or AKT kinases, c-kit inhibitors, abl kinase inhibitors, PI3 kinase inhibitors, Plt3 kinase inhibitors, CSF-1R kinase inhibitors, IGF receptor, kinase inhibitors; aurora kinase inhibitors and cyclin dependent kinase inhibitors such as CDK2 and/or CDK4 inhibitors; and CCR2, CCR4 or CCR6 antagonists; (v) antiangiogenic agents such as those which inhibit the effects of vascular endothelial growth factor, [for example the anti-vascular endothelial cell growth factor antibody bevacizumab (Avastin™)]; thalidomide; lenalidomide; and for example, a EGF receptor tyrosine kinase inhibitor such as vandetanib, vatalanib, sunitinib, axitinib and pazopanib; (vi) gene therapy approaches, including for example approaches to replace aberrant genes such as aberrant p53 or aberrant BRCA1 or BRCA2; (vii) immunotherapy approaches, including for example antibody therapy such as alemtuzumab, rituximab, ibritumomab tiuxetan (Zevalin®) and ofatumumab; interferons such as interferon α; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); gp100; dendritic cellbased vaccines (such as Ad.p53 DC); toll-like receptor modulators for example TLR-7 or TLR-9 agonists; PD-1, PD-L1, PD-L2 and CTL4-A modulators (for example Nivolumab), antibodies and vaccines; other IDO inhibitors (such as indoximod); anti-PD-1 monoclonal antibodies (such as MK-3475 and nivolumab); anti-PDL1 monoclonal antibodies (such as MEDI-4736 and RG-7446); anti-PDL2 monoclonal antibodies; and anti-CTLA-4 antibodies (such as ipilumumab; and (viii) cytotoxic agents for example fludaribine (fludara), cladribine, pentostatin (Nipent™); (ix) targeted therapies, for example PI3K inhibitors, for example idelalisib and perifosine; SMAC (second mitochondriaderived activator of caspases) mimetics, also known as Inhibitor of Apoptosis Proteins (IAP) antagonists (IAP antagonists). These agents act to suppress IAPs, for example XIAP, cIAPI and cIAP2, and thereby re-establish cellular apoptotic pathways. Particular SMAC mimetics include Birinapant (TL32711, TetraLogic Pharmaceuticals), LCL161 (Novartis), AEG40730 (Aegera Therapeutics), SM-164 (University of Michigan), LBW242 (Novartis), ML101 (Sanford-Burnham Medical Research Institute), AT-406 (Ascenta Therapeutics/University of Michigan), GDC-0917 (Genentech), EG35156 (Aegera Therapeutic), and HGS1029 (Human Genome Sciences); and agents which target ubiquitin proteasome system (UPS), for example, bortezomib, carfilzomib, marizomib (NPI-0052), MLN9708 and p53 agonists, for example Nutlin-3A (Roche) and MI713 (Sanofi); (xii) chimeric antigen receptors, anticancer vaccines and arginase inhibitors; and (xiii) DNA damage response inhibitors, for example ATM, ATR, CHK1, WEE1, BER or PARP inhibitors. For example, a PARP inhibitor (e.g. olaparib, veliparib, rucaparib or niraparib, BMN-673.

The additional anti-tumor agent may be a single agent or one or more of the additional agents listed herein. In some embodiments, the additional anti-tumor agent is used in combination with a compound or prodrug of the present disclosure and radiotherapy. In some embodiments, the additional anti-tumor agent is used in combination with the compound or prodrug of the present disclosure and a DNA damaging chemotherapeutic agent.

In some embodiments, the compound or prodrug of the present disclosure is for use in combination with a DNA damaging chemotherapeutic agent in the treatment of a cancer. The DNA damaging chemotherapeutic agent may be, for example, an alkylating agent, an antimetabolite and/or a topoisomerase inhibitor. In certain embodiments, the DNA damaging agent is an alkylating agent selected from: a platinum drug (e.g. cisplatin, oxaliplatin or carboplatin), cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine. In certain embodiments, the DNA damaging agent is an antimetabolite selected from: gemcitabine, 5-fluorouracil, tegafur, raltitrexed, methotrexate, pemetrexed, cytosine arabinoside, floxuridine, cytarabine, 6-mercaptopurine, 6-thioguanine, fludarabine phosphate, pentostatine and hydroxyurea. In certain embodiments, the DNA damaging agent topoisomerase inhibitor selected from epipodophyllotoxins like etoposide and teniposide, amsacrine, topotecan, irinotecan, mitoxantrone and camptothecin.

In certain embodiments, methods of treating cancer in a subject include administering a compound or prodrug of the present disclosure together with radiotherapy in the treatment of a cancer in the subject. In some cases, the compound or prodrug of the present disclosure acts to sensitize cancer cells, particularly hypoxic cancer cells to radiotherapy. Accordingly, embodiments of the present disclosure include a method of treating a cancer in a subject, the method comprising administering to a subject an effective amount of a compound or prodrug of the present disclosure, where the treatment of the subject further comprises radiotherapy. In some instances, the method includes administering a compound or prodrug of the present disclosure simultaneously, sequentially or separately with radiotherapy.

The compounds and prodrugs of the present disclosure may be used in combination with various forms of radiotherapy. In certain embodiments, the radiotherapy may be an external radiation therapy or an internal radiotherapy. External radiation therapy utilizes photons (e.g. X-rays), protons and/or electrons. The external radiation therapy may be administered using methods, for example, 3-D conformal radiation therapy, intensity-modulated radiation therapy, image-guided radiation therapy, tomotherapy, stereotactic radiosurgery, stereotactic body radiation therapy or proton-beam therapy. Internal radiotherapy utilizes a radioactive source inside the body. The internal radio therapy may take the form of a radioactive implant (brachytherapy) placed inside the body (e.g. interstitial brachytherapy or intracavity brachytherapy). The implant may take the form of radioactive pellets, seeds, sheets, wires or tubes that are placed in or close to the tumor to be treated. Internal radiotherapy may also be administered as a radioactive liquid, for example a liquid comprising radioactive iodine, radioactive strontium, radioactive phosphorus or radium 223.

In certain embodiments, the compound or prodrug of the present disclosure is administered substantially simultaneously with radiotherapy. In certain embodiments, the compound or prodrug of the present disclosure is administered to a subject that has received prior radiotherapy. For example, the compound or prodrug may be administered to a subject that has been treated with radiotherapy 1 hour, 2 hours, 4 hours 8 hours, 12 hours, 1 day, 2 days, 1 week, 2 weeks or 1 month prior to administration of the compound or prodrug. In certain embodiments the compound or prodrug is for use in the treatment of a cancer in a subject prior to the subject receiving radiotherapy. For example, the compound or prodrug may be administered to a subject 1 hour, 2 hours, 4 hours 8 hours, 12 hours, 1 day, 2 days, 1 week, 2 weeks or 1 month prior to initiating radiotherapy.

### Methods of Gene Editing

In some embodiments, methods of the present disclosure also include a method of repairing a DNA break in one or more target genomic regions via a homology directed repair (HDR) pathway. In some embodiments, the method includes administering to one or more cells that have one or more target genomic regions, a genome editing system and a compound of the present disclosure. The genome editing system interacts with a nucleic acid(s) of the target genomic regions, resulting in a DNA break, and wherein the DNA break is repaired at least in part via a HDR pathway.

In some embodiments, methods of the present disclosure also include a method of inhibiting or suppressing repair of a DNA break in one or more target genomic regions via a non-homologous end joining (NHEJ) pathway. In some embodiments, the method includes administering to one or more cells that have one or more target genomic regions, a genome editing system and a compound of the present disclosure. The genome editing system interacts with a nucleic acid of the one or more target genomic regions, resulting in a DNA break, and wherein repair of the DNA break via a NHEJ pathway is inhibited or suppressed.

In some embodiments, methods of the present disclosure also include a method of modifying expression of one or more genes or proteins. In some embodiments, the method includes administering to one or more cells that comprise one or more target genomic regions, a genome editing system and a compound of the present disclosure. The genome editing system interacts with a nucleic acid of the one or more target genomic regions of a target gene, resulting in editing the one or more target genomic regions and wherein the edit modifies expression of a downstream gene and/or protein associated with the target gene.

In some embodiments, methods of the present disclosure also include methods for editing a target genome, e.g., by correcting a mutation. Such methods can increase genome editing efficiency by the use of a DNA-PK inhibitor of the present disclosure.

A genomic editing system can stimulate or induce a DNA break, such as DSB at the desired locus in the genome (or target genomic region). The creation of DNA cleavage prompts cellular enzymes to repair the site of break through either the error prone NHEJ pathway or through the error-free HDR pathway. In NHEJ, the DNA lesion is repaired by fusing the two ends of the DNA break in a series of enzymatic processes involving Ku70/80 heterodimer and DNA dependent protein kinase (DNA-PK) enzymes. The repair mechanism involves tethering and alignment of two DNA ends, resection, elongation and ligation resulting in the formation of small insertion or deletion mutations (indels) at the break site. Indels introduced into the coding sequence of a gene can cause either premature stop codon or frame-shift mutations that lead to the production of nonfunctional, truncated proteins. The mechanism of HDR pathway is thought to involve a different set of repair proteins, such as Rad51, that stimulate strand invasion by a donor repair template for base insertion or gene replacement. Hence, HDR allows introduction of exogenous DNA template to obtain a desired outcome of DNA editing within a genome and can be a powerful strategy for translational disease modeling and therapeutic genome editing to restore gene function.

Of the two DNA repair pathways, NHEJ occurs at a much higher frequency and reports of more than 70% efficiency can be achieved even in neurons. The HDR gene correction, however, occurs at very low frequency and during S and G2 phase when DNA replication is completed and sister chromatids are available to serve as repair templates.

DNA protein-kinase (DNA-PK) plays a role in various DNA repair processes. DNA-PK participates in DNA double-stranded break repair through activation of the NHEJ pathway. NHEJ is thought to proceed through three steps: recognition of the DSBs, DNA processing to remove non-ligatable ends or other forms of damage at the termini, and finally ligation of the DNA ends. Recognition of the DSB is carried out by binding of the Ku heterodimer to the ragged DNA ends followed by recruitment of two molecules of DNA-dependent protein kinase catalytic subunit (DNA-PKcs) to adjacent sides of the DSB; this serves to protect the broken termini until additional processing enzymes are recruited. Recent data supports the hypothesis that DNA-PKcs phosphorylates the processing enzyme, Artemis, as well as itself to prepare the DNA ends for additional processing. In some cases, DNA polymerase may be required to synthesize new ends prior to the ligation step. The auto-phosphorylation of DNA-PKcs is believed to induce a conformational change that opens the central DNA binding cavity, releases DNA-PKcs from DNA, and facilitates the ultimate re-ligation of the DNA ends.

In some embodiments, methods of the present disclosure include methods to enhance gene editing, in particular increasing the efficiency of repair of DNA break via a HDR pathway, or the efficiency of inhibiting or suppressing repair of DNA break via a NHEJ pathway, in genome editing systems, including CRISPR-based HDR repair in cells. In some embodiments, a genome editing system administered to a cell may interact with a nucleic acid of the target gene, resulting in or causing a DNA break; such DNA break is repaired by several repair pathways, e.g., HDR, and a DNA-PK inhibitor administered to a cell inhibits, blocks, or suppresses a NHEJ repair pathway, and the frequency or efficiency of HDR DNA repair pathway can be increased or promoted.

The interaction between a genome editing system with a nucleic acid of the target gene can be hybridization of at least part of the genome editing system with the nucleic acid of the target gene, or any other recognition of the nucleic acid of the target gene by the genome editing system. In some embodiments, such interaction is a protein-DNA interaction or hybridization between base pairs.

In some embodiments, methods of the present disclosure include methods of editing one or more target genomic regions in a cell by administering to the cell a genome editing system and a DNA-PK inhibitor. The editing can occur simultaneously or sequentially. Editing of the one or more target genomic regions includes any kind of genetic manipulations or engineering of a cell's genome. In some embodiments, the editing of the one or more target genomic regions can include insertions, deletions, or replacements of genomic regions in a cell. Genomic regions comprise the genetic material in a cell, such as DNA, RNA, polynucleotides, and oligonucleotides. Genomic regions in a cell also comprise the genomes of the mitochondria or chloroplasts contained in a cell.

In some embodiments, the insertions, deletions or replacements can be either in a coding or a non-coding genomic region, in intronic or exonic regions, or any combinations thereof including overlapping or non-overlapping segments thereof. As used herein, a “non-coding region” refers to genomic regions that do not encode an amino acid sequence. For example, non-coding regions include introns. Coding regions refer to genomic regions that code for an amino acid sequence. For example, coding regions include exons.

In some embodiments, the editing of one or more target genomic regions can occur in any one or more target regions in a genome of a cell. In some embodiments, the editing of one or more target genomic regions can occur, for example, in an exon, an intron, a transcription start site, in a promoter region, an enhancer region, a silencer region, an insulator region, an antirepressor, a post translational regulatory element, a polyadenylation signal (e.g. minimal poly A), a conserved region, a transcription factor binding site, or any combinations thereof.

In some embodiments, administration to a cell with a DNA-PK inhibitor and a genomic editing system results in increased targeted genome editing efficiency as compared to conditions in which a DNA-PK inhibitor and a genomic editing system is not administered to a cell. In some embodiments, the increased editing efficiency is about 1-fold, 2-fold, 3-fold, 4-fold, 5-fold, 10-fold, 15-fold, 20-fold, 25-fold, 30-fold, 40-fold, 50-fold, or 100-fold, in comparison to a condition in which a DNA-PK inhibitor and a genome editing system is not administered to a cell, or compared to a condition in which only a genome editing system and not a DNA-PK inhibitor is administered to a cell. The efficiency of genomic editing can be measured by any method known in the art, for example, by any method that ascertains the frequency of targeted polynucleotide integration or by measuring the frequency of targeted mutagenesis. Targeted polynucleotide integrations can also result in alteration or replacement of a sequence in a genome, chromosome or a region of interest in cellular chromatin. Targeted polynucleotide integrations can result in targeted mutations including, but not limited to, point mutations (i.e., conversion of a single base pair to a different base pair), substitutions (i.e., conversion of a plurality of base pairs to a different sequence of identical length), insertions or one or more base pairs, deletions of one or more base pairs and any combination of the aforementioned sequence alterations.

In some embodiments, the methods of editing one or more target genomic regions in a cell involve administering to the cell a genome editing system and a DNA-PK inhibitor. In some embodiments, the cell is synchronized at the S or the G2 cell cycle phase. Synchronization of the cell at the S or G2 cell cycle phase can be achieved by any method known in the art. As a non-limiting example, agents that can be used to synchronize a cell at the S or G2 cell cycle phase include aphidicolin, dyroxyurea, lovastatin, mimosine, nocodazole, thymidine, or any combinations thereof. In some embodiments, the agents for cell synchronization can be administered at any time during the gene-editing process. In some embodiments, a cell can be synchronized at the S or the G2 phase of the cell cycle before, during, or after administering to a cell(s) a genome editing system and/or a DNA-PK inhibitor.

In some embodiments, the methods of editing one or more target genomic regions in a cell by administering to the cell a genome editing system and a DNA-PK inhibitor results in increased cell survival in comparison to conditions in which a genome editing system and a DNA-PK inhibitor were not administered to a cell, or in comparison to conditions in which only a gene editing system is contacted or administered into a cell(s) and not a DNA-PK inhibitor.

In some embodiments, methods of the present disclosure include methods of repairing a DNA break in one or more target genomic regions via an HDR pathway. The administering to a cell a genome editing system and a DNA-PK inhibitor results in a DNA break of a targeted region of the genome, and the DNA break is subsequently repaired, at least in part, by a HDR pathway. These methods result in increased amounts of HDR-mediated repair (e.g. HDR pathway) in the one or more target genomic regions resulting in greater efficiency of HDR-mediated repair as compared to conditions in which a DNA-PK inhibitor and a genomic editing system is not administered to a cell. In some embodiments, the efficiency of HDR pathway mediated repair of the DNA break is about 1-fold, 2-fold, 3-fold, 4-fold, 5-fold, 10-fold, 15-fold, 20-fold, 25-fold, 30-fold, 40-fold, 50-fold, or 100-fold, in comparison to a condition in which a DNA-PK inhibitor and a genome editing system is not administered to a cell, or compared to a condition in which only a genome editing system and not a DNA-PK inhibitor is administered to a cell. The efficiency of HDR pathway mediated repair can be measured by any method known in the art, for example, by ascertaining the frequency of targeted polynucleotide integration or by measuring the frequency of targeted mutagenesis.

In some embodiments, the methods herein provide for repairing the DNA break by increasing the efficiency of the HDR pathway. The HDR pathway can be “canonical” or “alternative.” “HDR” (homology directed repair) refers to a specialized form of DNA repair that takes place, for example, during repair of double-strand breaks or a DNA nick in a cell. HDR of double stranded breaks is generally based on nucleotide sequence homology, uses a “donor” molecule to template repair of a “target” molecule (e.g., the one that experienced the double-strand break), and can lead to the transfer of genetic information from the donor to the target. Canonical HDR of double stranded breaks is generally based on BRCA2 and RAD51 and typically employs a dsDNA donor molecule. Non-canonical, or “alternative,” HDR is an HDR mechanism that is suppressed by BRCA2, RAD51, and/or functionally-related genes. Alternative HDR may use a ssDNA or nicked dsDNA donor molecule.

In some embodiments, the methods of repairing a DNA break in one or more target genomic regions via an HDR pathway by administering to the cell a genome editing system and a DNA-PK inhibitor result in increased cell survival in comparison to conditions in which a genome editing system and a DNA-PK inhibitor are not administered to a cell, or in comparison to conditions in which only a gene editing system is administered to a cell and not a DNA-PK inhibitor.

In some embodiments, provided herein are methods of inhibiting or suppressing NHEJ-mediated repair of a DNA break in one or more target genomic regions in a cell. In some embodiments, the inhibiting or suppressing of NHEJ-mediated repair of a DNA break is performed by inhibiting or suppressing the NHEJ pathway. The NHEJ pathway can be either classical (“canonical”) or an alternative NHEJ pathway (alt-NHEJ, or microhomology-mediated end joining (MMEJ)). The NHEJ pathway or alt-NHEJ pathway is suppressed in a cell by administering to a cell a genome editing system and a DNA-PK inhibitor.

The classical NHEJ repair pathway is a DNA double stranded break repair pathway in which the ends of the double stranded break are ligated without extensive homology. Classical NHEJ repair uses several factors, including KU70/80 heterodimer (KU), XRCC4, Ligase IV, and DNA protein kinases catalytic subunit (DNA-PKcs). Alt-NHEJ is another pathway for repairing double strand breaks. Alt-NHEJ uses a 5-25 base pair microhomologous sequence during alignment of broken ends before joining the broken ends. Alt-NHEJ is largely independent of KU70/80 heterodimer (KU), XRCC4, Ligase IV, DNA protein kinases catalytic subunit (DNA-PKcs), RAD52, and ERCC1.

In some embodiments, the methods of inhibiting or suppressing NHEJ-mediated repair of a DNA break via the NHEJ pathway in one or more target genomic regions in a cell by inhibiting or suppressing the NHEJ pathway though the administering to a cell(s) a genomic editing system and a DNA-PK inhibitor result in increased efficiency of inhibiting or suppressing the NHEJ-mediated repair of the DNA break in comparison to a cell that have not received a genomic editing system and a DNA-PK inhibitor, or in comparison to a condition in which a cell receives a genomic editing system and not a DNA-PK inhibitor. In some embodiments, the increased efficiency of inhibiting or suppressing repair of a DNA break via the NHEJ pathway by contacting a cell with a DNA-PK inhibitor and a genome editing system is about 1-fold, 2-fold, 3-fold, 4-fold, 5-fold, 10-fold, 15-fold, 20-fold, 25-fold, 30-fold, 40-fold, 50-fold, or 100-fold, in comparison to a condition in which a DNA-PK inhibitor and a genome editing system is not administered to a cell, or compared to a condition in which only a genome editing system and not a DNA-PK inhibitor is administered to a cell. The efficiency inhibiting or suppressing repair of a DNA break via the NHEJ pathway can be measured by any method known in the art, for example, by ascertaining the frequency of targeted polynucleotide integration or by measuring the frequency of targeted mutagenesis.

In some embodiments, the methods of inhibiting or suppressing NHEJ-mediated repair of a DNA break in one or more target genomic regions in a cell by inhibiting or suppressing the NHEJ pathway though the administering to a cell a genomic editing system and a DNA-PK inhibitor result in increased cell survival in comparison to conditions in which a genome editing system and a DNA-PK inhibitor were not contacted or administered to a cell, or in comparison to conditions in which only a gene editing system is contacted or administered into a cell and not a DNA-PK inhibitor.

The DNA break can be a double stranded break (DSB) or two single stranded breaks (e.g. two DNA nicks). The DSB can be blunt ended or have either a 5′ or 3′ overhang, if the strands are each cleaved too far apart, the overhangs will continue to anneal to each other and exist as two nicks, not one DSB.

In some embodiments, methods of the present disclosure include methods of modifying expression of one or more genes (a target gene), and/or corresponding or downstream proteins, by administering to a cell a genome editing system and a DNA-PK inhibitor. In some embodiments, the genome editing system can create, for example, insertions, deletions, replacements, modification or disruption in a target genomic region of a target gene of the cell, resulting in modified expression of the target gene. In some embodiments, the insertion, deletions, replacement, modification or disruption can result in targeted expression of a specific protein, or group of proteins, or of downstream proteins. In some embodiments, the genome editing system can create insertions, deletions or replacements in non-coding regions or coding regions. In some embodiments, the genome editing system can create insertions, deletions, replacements, modification or disruption in a promoter region, enhancer region, and/or any other gene regulatory element, including an exon, an intron, a transcription start site, a silencer region, an insulator region, an antirepressor, a post translational regulatory element, a polyadenylation signal (e.g. minimal poly A), a conserved region, a transcription factor binding site, or any combinations thereof. In some embodiments, the genome editing system can create the insertions, deletions, replacements, modification or disruption in more than one target region, simultaneously or sequentially. In some embodiments, administering to a cell with a genome editing system and a DNA-PK inhibitor can allow for targeted modified gene expression in the cell. Such targeted modified gene expression can lead to expression of specific proteins and downstream proteins thereof.

In some embodiments, the expression of a downstream gene and/or protein is increased by at least 10%, 20%, 30%, 40%, 50%, 60%, 70%, 80%, 90%, 1, 1.5-fold, 2-fold, 2.5-fold, 3-fold, 3.5-fold, 4-fold, 4.5-fold, 5-fold, or 10-fold in comparison to a condition in which a DNA-PK inhibitor and a genome editing system is not administered to a cell, or compared to a condition in which only a genome editing system and not a DNA-PK inhibitor is administered to a cell.

In some embodiments, the gene expression of a downstream gene and/or protein is decreased by at least 10%, 20%, 30%, 40%, 50%, 60%, 70%, 80%, 90%, 95%, 96%, 97%, 98%, or 99% in comparison to a condition in which a DNA-PK inhibitor and a genome editing system is not administered to a cell, or compared to a condition in which only a genome editing system and not a DNA-PK inhibitor is administered to a cell.

The cell of the methods herein can be any cell. In some embodiments, the cell is a vertebrate cell. In some embodiments, the vertebrate cell is a mammalian cell. In some embodiment, the vertebrate cell is a human cell.

Various types of genome engineering systems can be used. The terms “genome editing system,” “gene editing system,” and the like, are used interchangeably herein, and refer to a system which edits a target gene or the function or expression thereof. In certain embodiments, a genome editing system comprises: at least one endonuclease component enabling cleavage of a target genomic region (or target sequence); and at least one genome-targeting element which brings or targets the endonuclease component to a target genomic region. Examples of genome-targeting element include a DNA-binding domain (e.g., zinc finger DNA-binding protein or a TALE DNA-binding domain), guide RNA elements (e.g., CRISPR guide RNA), and guide DNA elements (e.g., NgAgo guide DNA). Programmable genome-targeting and endonuclease elements enable precise genome editing by introducing DNA breaks, such as double strand breaks (DSBs) at specific genomic loci. DSBs subsequently recruit endogenous repair machinery for either non-homologous end-joining (NHEJ) or homology directed repair (HDR) to the DSB site to mediate genome editing.

Any genome editing system can be used in the methods of the present disclosure. In some embodiments, the genome editing system is a meganuclease based system, a zinc finger nuclease (ZFN) based system, a Transcription Activator-Like Effector-based Nuclease (TALEN) based system, a CRISPR-based system, or NgAgo-based system.

Meganuclease-based, ZFN-based and TALEN-based each comprise at least one DNA-binding domain or a nucleic acid comprising a nucleic acid sequence encoding the DNA-binding domain and achieve specific targeting or recognition of a target genomic region via protein-DNA interactions. A CRISPR-based system comprises at least one guide RNA element or a nucleic acid comprising a nucleic acid sequence encoding the guide RNA element and achieves specific targeting or recognition of a target genomic region via base-pairs directly with the DNA of the target genomic region. A NgAgo-based system comprises at least one guide DNA element or a nucleic acid comprising a nucleic acid sequence encoding the guide DNA element and achieves specific targeting or recognition of a target genomic region via base-pairs directly with the DNA of the target genomic region.

A Transcription Activator-Like Effector-based Nuclease (TALEN) system refers to a genome editing system that employs one or more Transcription Activator-Like Effector (TALE)-DNA binding domain and an endonuclease element, such as Fokl cleavage domain. The TALE-DNA binding domain comprises one or more TALE repeat units, each having 30-38 (such as, 31, 32, 33, 34, 35, or 36) amino acids in length. The TALE-DNA binding domain may employ a full-length TALE protein or fragment thereof, or a variant thereof. The TALE-DNA binding domain can be fused or linked to the endonuclease domain by a linker.

The terms “CRISPR-based system”, “CRISPR-based gene editing system”, “CRISPR-genome editing”, “CRISPR-gene editing”, “CRISPR-endonuclease based genome editing”, and the like, are used interchangeably herein, and collectively refer to a genome editing system that comprises one or more guide RNA elements; and one or more RNA-guided endonuclease elements. The guide RNA element comprises a targeter RNA comprising a nucleotide sequence substantially complementary to a nucleotide sequence at the one or more target genomic regions or a nucleic acid comprising a nucleotide sequence encoding the targeter RNA. The RNA-guided endonuclease element comprises an endonuclease that is guided or brought to a target genomic region by a guide RNA element; or a nucleic acid comprising a nucleotide sequence encoding such endonuclease.

Examples of such CRISPR-based gene editing system include, but are not limited to, a CRISPR-based system, such as a CRISPR-Cas system or a CRISPR-Cpf system.

In some embodiments, the CRISPR-based system is a CRISPR-Cas system. The CRISPR-Cas system comprises: (a) at least one guide RNA element or a nucleic acid comprising a nucleotide sequence encoding the guide RNA element, the guide RNA element comprising a targeter RNA that includes a nucleotide sequence substantially complementary to a nucleotide sequence at the one or more target genomic regions, and an activator RNA that includes a nucleotide sequence that is capable of hybridizing with the targeter RNA; and (b) a Cas protein element comprising a Cas protein or a nucleic acid comprising a nucleotide sequence encoding the Cas protein. The targeter RNA and activator RNAs can be separate or fused together into a single RNA.

In some embodiments, the CRISPR-based system includes Class 1 CRISPR and/or Class 2 CRISPR systems. Class 1 systems employ several Cas proteins together with a CRISPR RNAs (crRNA) as the targeter RNA to build a functional endonuclease. Class 2 CRISPR systems employ a single Cas protein and a crRNA as the targeter RNA. Class 2 CRISPR systems, including the type II Cas9-based system, comprise a single Cas protein to mediate cleavage rather than the multi-subunit complex employed by Class 1 systems. The CRISPR-based system also includes Class II, Type V CRISPR system employing a Cpf1 protein and a crRNA as the targeter RNA.

The Cas protein is a CRISPR-associated (Cas) double stranded nuclease. In some embodiments, CRISPR-Cas system comprises a Cas9 protein. In some embodiments, the Cas9 protein is SaCas9, SpCas9, SpCas9n, Cas9-HF, Cas9-H840A, FokI-dCas9, or D10A nickase. The term “Cas protein,” such as Cas9 protein, include wild-type Cas protein or functional derivatives thereof (such as truncated versions or variants of the wild-type Cas protein with a nuclease activity).

In some embodiments, the CRISPR-based system is a CRISPR-Cpf system. The “CRISPR-Cpf system” comprises: (a) at least one guide RNA element or a nucleic acid comprising a nucleotide sequence encoding the guide RNA element, the guide RNA comprising a targeter RNA having a nucleotide sequence complementary to a nucleotide sequence at a locus of the target nucleic acid; and (b) a Cpf protein element or a nucleic acid comprising a nucleotide sequence encoding the Cpf protein element.

An example of a Cpf protein element includes a Cpf1 nucleases, such as Francisella Cpf1 (FnCpf1) and any variants thereof. In some embodiments, the CRISPR-Cpf system employs a Cpf1-crRNA complex which cleaves target DNA or RNA by identification of a protospacer adjacent motif 5′-YTN-3-(where “Y” is a pyrimidine and “N” is any nucleobase) or 5′-TTN-3 in contrast to the G-rich PAM targeted by Cas9. After identification of PAM, Cpf1 introduces a sticky-end-like DNA double-stranded break of 4 or 5 nucleotides overhang.

In some embodiments, the genome editing system is aNgAgo-based system. The NgAgo-based system comprises at least one guide DNA element or a nucleic acid comprising a nucleic acid sequence encoding the guide DNA element; and a DNA-guided endonuclease. The NgAgo-based system employs DNA as a guide element. Its working principle is similar to that of CRISPR-Cas9 technology, but its guide element is a segment of guide DNA (dDNA) rather than gRNA in CRISPR-Cas9 technology. An example of DNA-guided endonuclease is an Argonaute endonuclease (NgAgo) from Natronobacterium gregoryi.

In some embodiments, the efficiency of the repair of the DNA break at the target genomic regions in the one or more cells via a HDR pathway is increased as compared to that in otherwise identical cell or cells but without the compound.

In some embodiments, the efficiency of inhibiting or suppressing the repair of the DNA break at the target genomic regions in the one or more cells via a NHEJ pathway is increased as compared to that in otherwise identical cell or cells but without the compound.

In some embodiments, the efficiency is increased by at least 2-fold, 3-fold, 4-fold, 5-fold, 10-fold, 15-fold, 20-fold, 25-fold, 30-fold, 40-fold, 50-fold, or 100-fold as compared to that in otherwise identical cell or cells but without compound.

In some embodiments, the efficiency is measured by frequency of targeted polynucleotide integration. In some embodiments, the efficiency is measured by frequency of targeted mutagenesis. In some embodiments, the targeted mutagenesis comprises point mutations, deletions, and/or insertions.

In some embodiments, the expression of a downstream gene and/or protein associated with the target gene is increased as compared to the baseline expression level in the one or more cells prior to the administration. For example, said expression is increased by at least 10%, 20%, 30%, 40%, 50%, 60%, 70%, 80%, 90%, 1-fold, 1.5-fold, 2-fold, 2.5-fold, 3-fold, 3.5-fold, 4-fold, 4.5-fold, 5-fold, or 10-fold as compared to the baseline expression level in the one or more cells prior to the administration.

In some embodiments, the expression of a downstream gene and/or protein associated with the target gene is decreased as compared to the baseline expression level in the one or more cells prior to the administration. For example, the gene expression is decreased by at least 10%, 20%, 30%, 40%, 50%, 60%, 70%, 80%, 90%, 95%, 96%, 97%, 98%, or 99% as compared to the baseline expression level in the one or more cells prior to the administration.

In some embodiments, the expression of a downstream gene and/or protein associated with the target gene is substantially eliminated in the one or more cells.

In some embodiments, the cell is synchronized at the S or the G2 cell cycle phase.

In some embodiments, the one or more cells that are administered or contacted with the compound have increased survival in comparison to one or more cells that have not been administered or contacted with the compound.

In some embodiments, the genome editing system and the compound are administered into the one or more cells simultaneously. In some embodiments, the genome editing system and the compound are administered into the one or more cells sequentially. In some embodiments, the genome editing system is administered into the one or more cells prior to the compound. In some embodiments, the compound is administered into the one or more cells prior to the genome editing system.

In some embodiments, the one or more cells are cultured cells. In some embodiments, the one or more cells are in vivo cells within an organism. In some embodiments, the one or more cells are ex vivo cells from an organism. In some embodiments, the organism is a mammal. In some embodiments, the organism is a human.

In certain embodiments, the compounds of the present disclosure find use in methods of treating a genetic disease, condition or disorder in a subject. In certain embodiments, the genetic disease, condition or disorder may be an acquired disease, condition or disorder (e.g., post-fetal development of the disorder or medical condition). In certain embodiments, the genetic disease, condition or disorder may be an inherited disease, condition or disorder. The inherited disease, condition or disorder may be the result from mutations or duplications in chromosomal regions (e.g. from point mutations, deletions, insertions, frameshift, chromosomal duplications or deletions). In some embodiments, the disease, condition or disorder may be selected from cancer, Down syndrome, Duchenne muscular dystrophy, fragile X syndrome, Friedreich's ataxia, hematological disorders (e.g., hemoglobinopathies including sickle cell disease and beta-thalassemia), Huntington's disease, juvenile myoclonic epilepsy, myotonic dystrophy, ophthalmological disorders (e.g., blindness, Leber congenital amaurosis), and spinocerebellar ataxias.

In some embodiments, the genome editing system and the compound are administered via a same route. In some embodiments, the genome editing system and the compound are administered via a different route. In some embodiments, the genome editing system is administered intravenously and the compound is administered orally.

### Pharmaceutical Compositions

In certain embodiments, the disclosed compounds and prodrugs thereof are useful for the treatment of a disease or disorder. Accordingly, pharmaceutical compositions comprising at least one disclosed compound or prodrug are also described herein. For example, the present disclosure provides pharmaceutical compositions that include a therapeutically effective amount of a compound or prodrug of the present disclosure (or a pharmaceutically acceptable salt or solvate or hydrate or stereoisomer thereof) and a pharmaceutically acceptable excipient.

A pharmaceutical composition that includes a subject compound (or prodrug) may be administered to a patient alone, or in combination with other supplementary active agents. For example, one or more compounds or prodrugs according to the present disclosure can be administered to a patient with or without supplementary active agents. The pharmaceutical compositions may be manufactured using any of a variety of processes, including, but not limited to, conventional mixing, dissolving, granulating, dragee-making, levigating, emulsifying, encapsulating, entrapping, lyophilizing, and the like. The pharmaceutical composition can take any of a variety of forms including, but not limited to, a sterile solution, suspension, emulsion, spray dried dispersion, lyophilisate, tablet, microtablets, pill, pellet, capsule, powder, syrup, elixir or any other dosage form suitable for administration.

A compound or prodrug of the present disclosure may be administered to a subject using any convenient means capable of resulting in the desired reduction in disease condition or symptom. Thus, a compound or prodrug can be incorporated into a variety of formulations for therapeutic administration. More particularly, a compound or prodrug can be formulated into pharmaceutical compositions by combination with appropriate pharmaceutically acceptable excipients, carriers or diluents, and may be formulated into preparations in solid, semi-solid, liquid or gaseous forms, such as tablets, capsules, powders, granules, ointments, solutions, suppositories, injections, inhalants, aerosols, and the like.

Formulations for pharmaceutical compositions are described in, for example, Remington's Pharmaceutical Sciences, by E. W. Martin, Mack Publishing Co., Easton, Pa., 19th Edition, 1995, which describes examples of formulations (and components thereof) suitable for pharmaceutical delivery of the disclosed compounds. Pharmaceutical compositions that include at least one of the compounds or prodrugs can be formulated for use in human or veterinary medicine. Particular formulations of a disclosed pharmaceutical composition may depend, for example, on the mode of administration and/or on the location of the subject to be treated. In some embodiments, formulations include a pharmaceutically acceptable excipient in addition to at least one active ingredient, such as a compound of the present disclosure. In other embodiments, other medicinal or pharmaceutical agents, for example, with similar, related or complementary effects on the disease or condition being treated can also be included as active ingredients in a pharmaceutical composition.

Pharmaceutically acceptable carriers useful for the disclosed methods and compositions may depend on the particular mode of administration being employed. In addition to biologically neutral carriers, pharmaceutical compositions to be administered can optionally contain non-toxic auxiliary substances (e.g., excipients), such as wetting or emulsifying agents, preservatives, and pH buffering agents, and the like. The disclosed pharmaceutical compositions may be formulated as a pharmaceutically acceptable salt of a disclosed compound.

The term “unit dosage form,” as used herein, refers to physically discrete units suitable as unitary dosages for human and animal subjects, each unit containing a predetermined quantity of a compound or prodrug calculated in an amount sufficient to produce the desired effect in association with a pharmaceutically acceptable diluent, excipient, carrier or vehicle. The specifications for a compound or prodrug depend on the particular compound or prodrug employed and the effect to be achieved, and the pharmacodynamics associated with each compound in the subject.

The dosage form of a disclosed pharmaceutical composition may be determined by the mode of administration chosen. For example, in addition to injectable fluids, topical or oral dosage forms may be employed. Topical preparations may include eye drops, ointments, sprays and the like. Oral formulations may be liquid (e.g., syrups, solutions or suspensions), or solid (e.g., powders, pills, tablets, or capsules). Methods of preparing such dosage forms are known, or will be apparent, to those skilled in the art.

Certain embodiments of the pharmaceutical compositions that include a subject compound or prodrug may be formulated in unit dosage form suitable for individual administration of precise dosages. The amount of active ingredient administered may depend on the subject being treated, the severity of the affliction, and the manner of administration, and is known to those skilled in the art. In certain instances, the formulation to be administered contains a quantity of the compound or prodrug disclosed herein in an amount effective to achieve the desired effect in the subject being treated.

Each therapeutic compound can independently be in any dosage form, such as those described herein, and can also be administered in various ways, as described herein. For example, the compounds or prodrugs may be formulated together, in a single dosage unit (that is, combined together in one form such as capsule, tablet, powder, or liquid, etc.) as a combination product. Alternatively, when not formulated together in a single dosage unit, an individual compound or prodrug may be administered at the same time as another therapeutic compound or sequentially, in any order thereof.

A disclosed compound can be administered alone, as the sole active pharmaceutical agent, or in combination with one or more additional compounds or prodrugs of the present disclosure or in conjunction with other agents. When administered as a combination, the therapeutic agents can be formulated as separate compositions that are administered simultaneously or at different times, or the therapeutic agents can be administered together as a single composition combining two or more therapeutic agents. Thus, the pharmaceutical compositions disclosed herein containing a compound of the present disclosure optionally include other therapeutic agents. Accordingly, certain embodiments are directed to such pharmaceutical compositions, where the composition further includes a therapeutically effective amount of an agent selected as is known to those of skill in the art.

### Methods of Administration

The subject compounds or prodrugs find use for treating a disease or disorder in a subject. The route of administration may be selected according to a variety of factors including, but not limited to, the condition to be treated, the formulation and/or device used, the subject to be treated, and the like. Routes of administration useful in the disclosed methods include, but are not limited to, oral and parenteral routes, such as intravenous (iv), intraperitoneal (ip), rectal, topical, ophthalmic, nasal, intrathecal, and transdermal. Formulations for these dosage forms are described herein.

An effective amount of a subject compound or prodrug may depend, at least, on the particular method of use, the subject being treated, the severity of the affliction, and the manner of administration of the therapeutic composition. A “therapeutically effective amount” of a composition is a quantity of a specified compound or prodrug sufficient to achieve a desired effect in a subject (e.g., patient) being treated. For example, this may be the amount of a subject compound necessary to prevent, inhibit, reduce or relieve a disease or disorder in a subject. Ideally, a therapeutically effective amount of a compound or prodrug is an amount sufficient to prevent, inhibit, reduce or relieve a disease or disorder in a subject without causing a substantial cytotoxic effect on host cells in the subject.

Therapeutically effective doses of a subject compound or prodrug or pharmaceutical composition can be determined by one of skill in the art. For example, in some instances, a therapeutically effective dose of a compound or prodrug or pharmaceutical composition is administered with a goal of achieving local (e.g., tissue) concentrations that are at least as high as the EC₅₀ of an applicable compound disclosed herein.

The specific dose level and frequency of dosage for any particular subject may be varied and may depend upon a variety of factors, including the activity of the subject compound or prodrug, the metabolic stability and length of action of that compound or prodrug, the age, body weight, general health, sex and diet of the subject, mode and time of administration, rate of excretion, drug combination, and severity of the condition of the host undergoing therapy.

In some embodiments, multiple doses of a compound or prodrug are administered. The frequency of administration of a compound can vary depending on any of a variety of factors, e.g., severity of the symptoms, condition of the subject, etc. For example, in some embodiments, a compound is administered once per month, twice per month, three times per month, every other week, once per week (qwk), twice per week, three times per week, four times per week, five times per week, six times per week, every other day, daily (qd/od), twice a day (bds/bid), or three times a day (tds/tid), etc.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. By “average” is meant the arithmetic mean. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### LIST OF EXAMPLES

Example 1: general procedures for synthesizing compounds; see page 160
Examples 2-4: organic syntheses of compounds; see pages 161, 220, 307
Examples 5-6: IC50 data; see pages 429, 435
Examples 7-8: EC50 data; see pages 439, 441
Example 9: Prodrug activation & EC50 data; see page 443
Examples 10-11: microsome analysis; see pages 452, 455
Example 12: Traffic Light Reporter (TLR) assay; see page 456
Example 13: CRISPR inactivation of DNA-PK to demonstrate direct activity of
DNA-PK inhibitors on HDR (Homology Directed Repair); see page 457
Example 14: pharmacokinetic (PK) studies; see page 458
Example 15: activity in in vivo biological assays; see page 466

### Example 1: General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any purification protocol known in the art, including chromatography, such as HPLC, preparative thin layer chromatography, flash column chromatography and ion exchange chromatography. Any suitable stationary phase can be used, including normal and reversed phases as well as ionic resins. In certain embodiments, the disclosed compounds are purified via silica gel and/or alumina chromatography. See, e.g., Introduction to Modem Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4^{th} edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds, including compounds that are not commercially available, can be synthesized via a variety of different synthetic routes using commercially available starting materials and/or starting materials prepared by conventional synthetic methods. A variety of examples of synthetic routes that can be used to synthesize the compounds disclosed herein are described in the schemes below.

### Example 2: Syntheses of Compounds

### A General Synthetic Procedure.

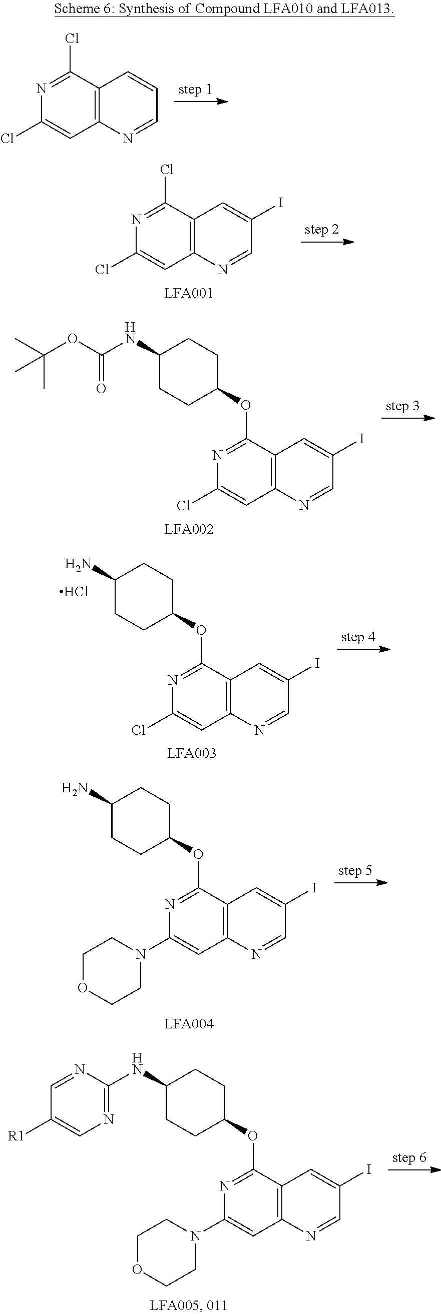

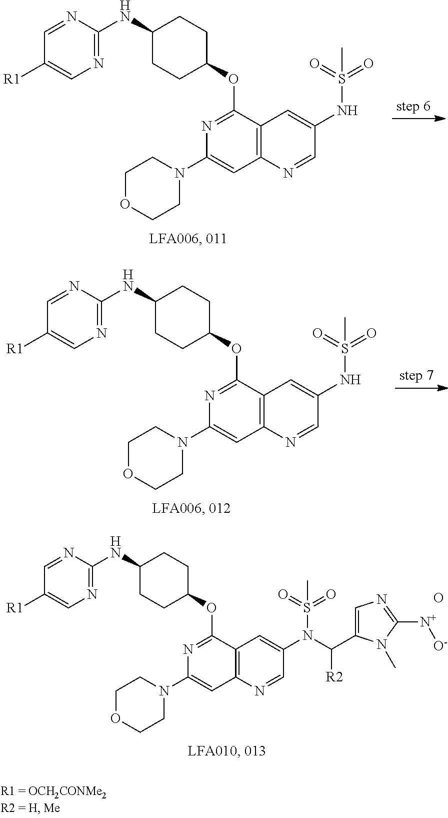

### Step 1

### 5,7-Dichloro-3-iodo-1,6-naphthyridine (LFA001)

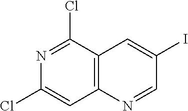 [SMILES: Clc1cc2ncc(I)cc2c(Cl)n1]

5,7-Dichloro-1,6-naphthyridine (5.25 g 26.4 mmol) was charged to a 10 mL RB flask, suspended/dissolved in acetic acid (85 mL) and N-Iodosuccinimide (10.98 g, 48.8 mmol) was added in one portion. The suspension was heated at 100° C. with stirring for 4 h and then cooled to room temperature and concentrated. The residue was dissolved in EtOAc (100 mL) and washed with sat. sodium metabisulphite solution (aq.) (150 mL). The layers were separated, the aqueous extracted with EtOAc (2×50 mL) and the combined organics washed with brine (100 mL), dried over Na₂SO₄, filtered and concentrated. The resulting residue was purified via automated flash chromatography using 0-10% EtOAc in n-heptane as the mobile phase to afford the titled compound as a pale yellow solid (5.23 g, 59%).

¹H NMR (400 MHz, CDCl₃) δ 9.22 (d, J=2.1 Hz, 1H), 8.94 (dd, J=2.0, 1.0 Hz, 1H), 7.89 (d, J=0.9 Hz, 1H).

m/z calcd. for C₈H₃Cl₂IN₂=323.8. Found [M+H]⁺=324.9 (for ³⁵Cl isotope).

### Step 2

### tert-Butyl N-[4-[(7-chloro-3-iodo-1,6-naphthyridin-5-yl)oxy]cyclohexyl]carbamate (LFA002)

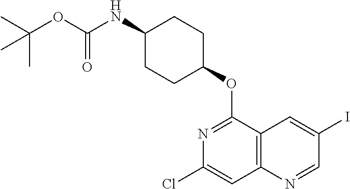 [SMILES: CC(C)(C)OC(=O)N[C@H]1CC[C@@H](Oc2nc(Cl)cc3ncc(I)cc23)CC1]

5,7-dichloro-3-iodo-1,6-naphthyridine (1.00 g, 3.08 mmol), tert-butyl N-(4-hydroxycyclohexyl)carbamate (1.33 g, 6.16 mmol) and cesium carbonate (5.01 g, 15.4 mmol) were charged to a 50 mL RB flask and anhydrous 1,4-dioxane (19 mL) was added. The resultant suspension was heated to 110° C. under an argon atmosphere for 24 h and then cooled to room temperature. The mixture was partitioned between water (100 mL) and EtOAc (3×50 mL) and the combined organics washed with brine (50 mL), dried over Na₂SO₄, filtered and concentrated. The resulting residue was purified via automated flash chromatography using 0-20% EtOAc in n-heptane as the mobile phase to afford the titled compound as a pale yellow solid (1.17 g, 75%).

¹H NMR (400 MHz, CDCl₃) δ 9.12 (d, J=2.2 Hz, 1H), 8.77 (dd, J=2.2, 0.9 Hz, 1H), 7.44 (d, J=0.9 Hz, 1H), 5.56-5.39 (m, 1H), 4.59 (br. s, 1H), 3.63 (br. s, 1H), 2.19-2.01 (m, 2H), 1.91 (dt, J=12.5, 4.2 Hz, 2H), 1.87-1.77 (m, 2H), 1.72-1.59 (m, 2H), 1.46 (s, 9H).

m/z calcd. for C₁₉H₂₃ClIN₃O₃=503.0. Found [M+H]⁺=504.0 (for ³⁵Cl isotope).

### Step 3

### 4-[(7-Chloro-3-iodo-1,6-naphthyridin-5-yl)oxy]cyclohexanamine; hydrochloride (LFA003)

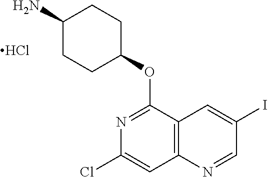 [SMILES: Cl ; N[C@H]1CC[C@@H](Oc2nc(Cl)cc3ncc(I)cc23)CC1]

Hydrochloric acid (3.7-4.3 M in 1,4-dioxane) (16.1 mL, 59.6 mmol) was added to a stirred solution of tert-butyl N-[4-[(7-chloro-3-iodo-1,6-naphthyridin-5-yl)oxy]cyclohexyl]carbamate (3.00 g, 5.96 mmol) in MeOH (30 mL). The mixture was stirred at room temperature for 24 h. The mixture was concentrated, and the residue was azeotroped with MeCN (30 mL) and dried in vacuo to afford the titled compound as a yellow solid (3.01 g, 103%).

¹H NMR (400 MHz, DMSO-d₆) δ 9.25 (d, J=2.1 Hz, 1H), 8.89 (dd, J=2.1, 0.9 Hz, 1H), 8.16 (s, 3H), 7.54 (d, J=0.9 Hz, 1H), 5.48-5.39 (m, 1H), 3.23-3.10 (m, 1H), 2.16-2.09 (m, 2H), 1.93-1.69 (m, 6H).

m/z calcd. for C₁₄H₁₅ClIN₃O=403.0. Found [M+H]+=404.0 (for ³⁵Cl isotope of free base).

### Step 4

### 4-[(3-Iodo-7-morpholino-1,6-naphthyridin-5-yl)oxy]cyclohexanamine (LFA004)

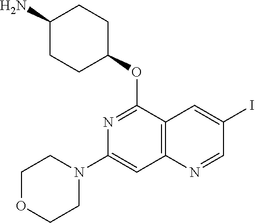 [SMILES: N[C@H]1CC[C@@H](Oc2nc(N3CCOCC3)cc3ncc(I)cc23)CC1]

The material was split into three equal batches of 0.871 g of starting material and 12 mL morpholine and heated in the microwave separately. The batches were then combined for work-up and purification. The procedure below is written as if the reaction was performed in a single batch.

A mixture of 4-[(7-chloro-3-iodo-1,6-naphthyridin-5-yl)oxy]cyclohexanamine; hydrochloride (2.62 g, 5.96 mmol) and morpholine (36.0 mL, 417 mmol) was heated in the microwave at 200° C. for 1 h. The mixture was concentrated and most of the residual morpholine was removed by azeotroping with toluene (2×20 mL). The crude product was purified via automated flash chromatography using 0-10% 2 M NH₃/MeOH in CH₂Cl₂ as the mobile phase to afford an orange solid (2.11 g). The solid was suspended in TBME (30 mL) and stirred vigorously for 30 minutes. The suspension was filtered in vacuo and the collected solid was washed with TBME (2×30 mL) and dried to give the titled compound as a yellow solid (1.87 g, 69%).

¹H NMR (400 MHz, DMSO-d₆) δ 8.89 (d, J=2.2 Hz, 1H), 8.52 (d, J=2.2 Hz, 1H), 6.48 (s, 1H), 5.44-5.21 (m, 1H), 3.76-3.66 (m, 4H), 3.54-3.45 (m, 4H), 2.75 (tt, J=8.9, 3.7 Hz, 1H), 2.11-1.96 (m, 2H), 1.73-1.57 (m, 4H), 1.57-1.41 (m, 2H). NH₂ not directly observed.

m/z calcd. for C₁₈H₂₃IN₄O₂=454.1. Found [M+H]⁺=455.1.

### Step 5

A microwave vial was charged with 4-[(3-iodo-7-morpholino-1,6-naphthyridin-5-yl)oxy]cyclohexanamine LFA004, the appropriate pyrimidine chloride (1.5-2.5 eq), triethylamine (2.0-3.0 eq) and EtOH (0.22 M). The vial was sealed, placed in a microwave reactor, and eradiated with microwaves for 2-12 h at 150° C. The mixture was concentrated and partitioned between sat. NaHCO₃ (aq.) and CH₂Cl₂ (3×). The combined organics were dried by passing through a hydrophobic frit and concentrated. The crude product was purified via automated flash chromatography using EtOAc/n-heptane or MeOH/CH₂Cl₂ as the mobile phase to afford compound LFA011.

### Step 6

A microwave vial was charged with the appropriate aryl iodide LFA011, methanesulfonamide (1.1 eq), copper(I) iodide (0.15 eq), trans-N,N′-dimethylcyclohexane-1,2-diamine (0.3 eq), potassium carbonate (2.0 eq) and 1,4-dioxane (0.05-0.13 M). The vial was sealed, and the contents purged with argon. The mixture was heated at 120° C. (conventional heating; bath temperature) for 4-24 h. The reaction mixture was filtered, rinsing with 5% MeOH/CH₂Cl₂ and the filtrate was concentrated. The residue was dissolved in MeOH, acidified with 4 M HCl in 1,4-dioxane and loaded onto an SCX cartridge The SCX cartridge was washed with MeOH and the product eluted with 2 M ammonia in MeOH. The relevant fraction was concentrated to give the crude product. The crude product was purified via automated flash chromatography using EtOAc/n-heptane or CH₂Cl₂/MeOH as the mobile phase and/or via automated reverse phase preparative HPLC using 5-95% 0.005 M NH₄OH/MeCN in 0.005 M NH₄OH/H₂O or 5-95% 0.1% HCOOH/MeCN in 0.1% HCOOH/H₂O as the mobile phase to afford compound LFA012.

### Step 7

An RB flask was charged with the appropriate aryl sulfonamide LFA012, alkyl halide LFA007 or LFA009 (1.1 eq), potassium carbonate (2.1 eq) and DMF (0.06 M). The suspension was stirred at room temperature under an argon atmosphere for 18-24 h. The mixture was diluted with CH₂Cl₂, filtered through Celite and concentrated. The crude product was purified via automated flash chromatography using EtOAc/n-heptane or MeOH/CH₂Cl₂ as the mobile phase and/or via automated reverse phase preparative HPLC using 5-95% 0.005 M NH₄OH/MeCN in 0.005 M NH₄OH/H₂O or 5-95% 0.1% HCOOH/MeCN in 0.1% HCOOH/H₂O as the mobile phase to afford compounds LFA010 and LFA013.

### 5-(Bromomethyl)-1-methyl-2-nitro-imidazole (LFA007)

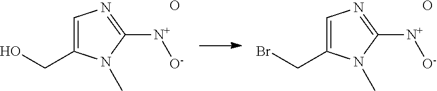

Dibromo(triphenyl)-λ\hat{ }5-phosphane (2.36 g, 5.60 mmol) was added as a suspension in CH₂Cl₂ (10 mL) to an ice-cooled stirred suspension of (3-methyl-2-nitro-imidazol-4-yl)methanol (0.80 g, 5.09 mmol) and DIPEA (1.77 mL, 10.2 mmol) in CH₂Cl₂ (15 mL). The mixture was stirred at 0-5° C. for 2 h, then allowed to warm to room temperature and stirred for 22 h. The mixture was adsorbed directly onto silica gel and was purified via automated flash chromatography using 0-60% EtOAc/n-heptane as the mobile phase to afford the titled compound as a pale yellow solid (0.71 g, 63%).

¹H NMR (400 MHz, CDCl₃) δ 7.20 (d, J=0.5 Hz, 1H), 4.47 (d, J=0.6 Hz, 2H), 4.04 (s, 3H).

m/z calcd. for C₅H₆BrN₃O₂=219.0. Found [M+H]+=220.1 (for ⁷⁹Br isotope).

### 5-(1-Chloroethyl)-1-methyl-2-nitro-imidazole (LFA009)

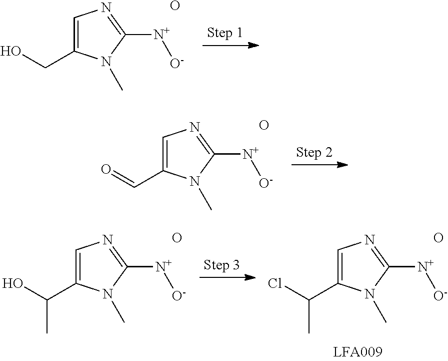

### Step 1

Dess-Martin periodinane (6.48 g, 15.3 mmol) was added to a stirred solution of (3-methyl-2-nitro-imidazol-4-yl)methanol (2.00.g, 12.7 mmol) in CH₂Cl₂ (30 mL). The mixture was stirred at room temperature for 16 h. The mixture was filtered to remove precipitated solids and the filtrate was washed 10 wt % Na₂S₂O₃ (aq.) (100 mL) and sat. NaHCO₃ (aq.) (100 mL). The combined organic phases were dried over Na₂SO₄, filtered, and concentrated to give the crude product as a yellow oil/gum which solidified on standing (2.268 g). The crude product was purified via automated flash chromatography using 0-60% EtOAc/n-heptane as the mobile phase to afford 3-methyl-2-nitro-imidazole-4-carbaldehyde as an off-white solid (1.43 g, 72%).

¹H NMR (400 MHz, CDCl₃) δ 9.93 (s, 1H), 7.81 (s, 1H), 4.36 (s, 3H).

m/z calcd. for C₅H₅N₃O₃=155.0. Found [M+H]⁺=156.2.

### Step 2

Titanium(IV) chloride (1.0 M in CH₂Cl₂) (10.6 mL, 10.6 mmol) was stirred in a dry ice/acetone cooling bath (internal temperature −72° C.). Methyl magnesium bromide (3.0 M in diethyl ether) (3.55 mL, 10.6 mmol) was added dropwise and the reaction allowed to warm to −45° C. The TiCl₄/MeMgBr solution was added dropwise to a solution of 3-methyl-2-nitro-imidazole-4-carbaldehyde (0.55 g, 3.55 mmol) in CH₂Cl₂ (15 mL) and stirring was continued at −45° C. rising to −30° C. for 3 h. The mixture was quenched with sat. NH₄Cl (aq.) (10 mL) and then diluted with CH₂Cl₂ and water. The layers were separated and the aqueous extracted with CH₂Cl₂. The combined organic phases were dried over Na₂SO₄, filtered and concentrated. The resulting reside was purified via automated flash chromatography using 0-5% MeOH/CH₂Cl₂ as the mobile phase to afford 1-(3-methyl-2-nitro-imidazol-4-yl)ethanol as a brown solid (0.19 g, 32%)

¹H NMR (400 MHz, CDCl₃) δ 7.07 (d, J=0.7 Hz, 1H), 4.93 (qd, J=6.5, 0.7 Hz, 1H), 4.09 (s, 3H), 1.71 (d, J=6.6 Hz, 3H). OH not directly observed.

m/z calcd. for C₆H₉N₃O₃=171.1. Found [M+H]⁺=172.2.

### Step 3

Triethylamine (0.09 mL, 0.68 mmol) was added to an ice-cooled stirred solution of 1-(3-methyl-2-nitro-imidazol-4-yl)ethanol (0.058 g, 0.34 mmol) in CH₂Cl₂ (4 mL). Methanesulfonyl chloride (0.04 mL, 0.51 mmol) was added and the reaction allowed to warm to room temperature and stirred for 18 h. The reaction mixture was diluted with CH₂Cl₂ (20 mL) and washed with sat. NaHCO₃ (aq.) (20 mL). The aqueous phase was extracted with CH₂Cl₂ (2×20 mL). The combined organic phases were dried by passing through a hydrophobic frit and concentrated to give the crude product as a yellow oil/gum (0.13 g). The crude product was purified via automated flash chromatography using 0-50% EtOAc/n-heptane as the mobile phase to afford the titled compound, 5-(1-chloroethyl)-1-methyl-2-nitro-imidazole LFA009, as a yellow oil (0.061 g, 95%).

¹H NMR (400 MHz, CDCl₃) δ 7.18 (d, J=0.7 Hz, 1H), 5.05 (qd, J=6.9, 0.7 Hz, 1H), 4.07 (s, 3H), 2.01 (d, J=6.8 Hz, 3H).

m/z calcd. C₆H₈ClN₃O₂=189.0. Found [M+H]⁺=190.2 (for ³⁵Cl isotope).

### 2-((2-Chloropyrimidin-5-yl)oxy)-N,N′-dimethylacetamide (7-i)

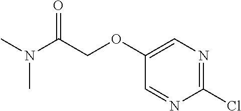

Prepared according to general procedure D (see below) starting with 2-chloropyrimidin-5-ol and 2-chloro-N,N′-dimethylacetamide to afford the titled compound in 97% yield. ¹H NMR (400 MHz, DMSO-d₆) δ 8.50 (s, 2H), 5.08 (s, 2H), 2.96 (s, 3H), 2.85 (s, 3H). m/z calcd. for C₈H₁₀ClN₃O₂=215.1. Found [M+H]⁺=216.1.

### 2-[2-[[4-[(3-Iodo-7-morpholino-1,6-naphthyridin-5-yl)oxy]cyclohexyl]amino]pyrimidin-5-yl]oxy-N,N′-dimethyl-acetamide (LFA011)

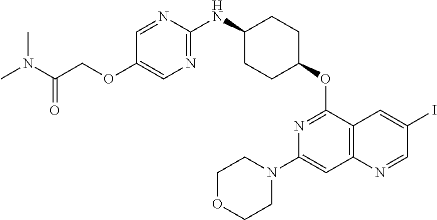 [SMILES: CN(C)C(=O)COc1cnc(N[C@H]2CC[C@@H](Oc3nc(N4CCOCC4)cc4ncc(I)cc34)CC2)nc1]

Prepared according to step 5 of Scheme 6 starting with LFA004, 2-(2-chloropyrimidin-5-yl)oxy-N,N′-dimethyl-acetamide 7-i (1.5 eq), triethylamine (2.0 eq) and heating for 12 h to afford the titled compound in 18% yield.

¹H NMR (400 MHz, CDCl₃) δ 8.86 (d, J=2.2 Hz, 1H), 8.58 (dd, J=2.3, 0.8 Hz, 1H), 8.11 (s, 2H), 6.44 (d, J=0.9 Hz, 1H), 5.39 (dt, J=4.9, 2.2 Hz, 1H), 5.03 (d, J=7.9 Hz, 1H), 4.64 (s, 2H), 3.95-3.86 (m, 1H), 3.87-3.80 (m, 4H), 3.57-3.51 (m, 4H), 3.05 (s, 3H), 2.99 (s, 3H), 2.20-2.10 (m, 2H), 2.03-1.92 (m, 2H), 1.89-1.79 (m, 2H), 1.79-1.68 (m, 2H).

m/z calcd. C₂₆H₃₂IN₇04=633.2. Found [M+H]⁺=634.2.

### 2-[2-[[4-[[3-(Methanesulfonamido)-7-morpholino-1,6-naphthyridin-5-yl]oxy]cyclohexyl]amino]pyrimidin-5-yl]oxy-N,N′-dimethyl-acetamide (LFA012)

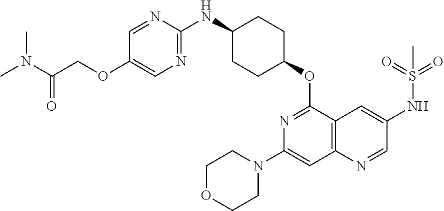 [SMILES: CN(C)C(=O)COc1cnc(N[C@H]2CC[C@@H](Oc3nc(N4CCOCC4)cc4ncc(NS(C)(=O)=O)cc34)CC2)nc1]

Prepared according to step 6 of Scheme 6 starting with LFA011, methanesulfonamide (2.2 eq), copper(I) iodide (0.30 eq), trans-N,N′-dimethylcyclohexane-1,2-diamine (0.6 eq), potassium carbonate (4.0 eq) and heating for 9 h to afford the titled compound in 44% yield.

¹H NMR (400 MHz, DMSO-d₆) δ 9.89 (br. s, 1H), 8.69 (d, J=2.7 Hz, 1H), 8.11 (dd, J=2.6, 0.8 Hz, 1H), 8.09 (s, 2H), 6.75 (d, J=7.4 Hz, 1H), 6.53 (d, J=0.9 Hz, 1H), 5.40-5.26 (m, 1H), 4.77 (s, 2H), 3.83-3.69 (m, 5H), 3.53-3.43 (m, 4H), 3.02 (s, 3H), 2.95 (s, 3H), 2.83 (s, 3H), 2.17-2.04 (m, 2H), 1.88-1.62 (m, 6H).

m/z calcd. C₂₇H₃₆N₈O₆S=600.2. Found [M+H]⁺=601.2.

### N,N-Dimethyl-2-[2-[[4-[[3-[(1-methyl-5-nitro-imidazol-2-yl)methyl-methylsulfonyl-amino]-7-morpholino-1,6-naphthyridin-5-yl]oxy]cyclohexyl]amino]pyrimidin-5-yl]oxy-acetamide (LFA013)

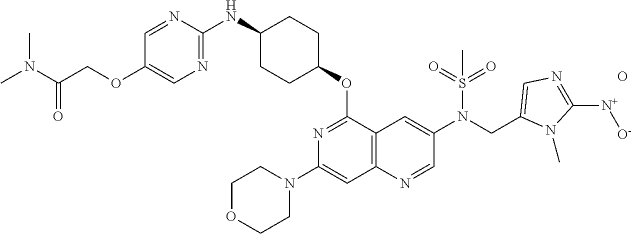

Prepared according to step 7 of Scheme 6 starting with LFA012, LFA007 (1.1 eq), potassium carbonate (2.1 eq) and stirring for 22 h to afford the titled compound in 32% yield.

¹H NMR (400 MHz, DMSO-d₆) δ 8.70 (d, J=2.6 Hz, 1H), 8.19 (dd, J=2.6, 0.8 Hz, 1H), 8.10 (s, 2H), 6.98 (s, 1H), 6.71 (d, J=7.6 Hz, 1H), 6.51 (d, J=0.9 Hz, 1H), 5.42-5.29 (m, 1H), 5.15 (s, 2H), 4.77 (s, 2H), 3.96 (s, 3H), 3.85-3.76 (m, 1H), 3.75-3.68 (m, 4H), 3.54-3.48 (m, 4H), 3.25 (s, 3H), 2.95 (s, 3H), 2.83 (s, 3H), 2.13-1.99 (m, 2H), 1.78 (t, J=12.6 Hz, 6H).

m/z calcd. C₃₂H₄₁N₁₁O₈S=739.3. Found [M+H]⁺=740.2.

### N-[4-[(3-Iodo-7-morpholino-1,6-naphthyridin-5-yl)oxy]cyclohexyl]pyrimidine-2-carboxamide (LFA014)

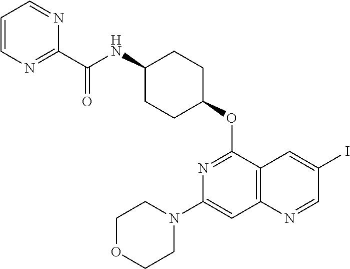 [SMILES: O=C(N[C@H]1CC[C@@H](Oc2nc(N3CCOCC3)cc3ncc(I)cc23)CC1)c1ncccn1]

T3P (50 wt % in EtOAc) (0.55 mL, 0.93 mmol) was added to an ice-cooled stirred suspension of 4-[(3-iodo-7-morpholino-1,6-naphthyridin-5-yl)oxy]cyclohexanamine LFA004 (0.296 g, 0.62 mmol), pyrimidine-2-carboxylic acid (0.084 g, 0.68 mmol) and DIPEA (0.35 mL, 1.98 mmol) in CH₂Cl₂ (6 mL) under an argon atmosphere. The suspension was allowed to stir at room temperature for 3.5 h and then left standing for 16 h. The reaction mixture was diluted with CH₂Cl₂ (5 mL) washed with sat. NaHCO₃ (aq.) (10 mL). The aqueous phase was washed with CH₂Cl₂ (2×5 mL). The combined organics were dried by passing through a hydrophobic frit and concentrated to afford the crude product as a yellow oil (117 mg). The crude product was purified via automated flash chromatography using 0-3% MeOH/CH₂Cl₂ as the mobile phase to afford the titled compound as a yellow gum (0.210 g, 60%).

¹H NMR (400 MHz, CDCl₃) δ 8.87 (d, J=4.9 Hz, 2H), 8.84 (d, J=2.2 Hz, 1H), 8.57 (dd, J=2.3, 0.8 Hz, 1H), 8.08 (d, J=8.5 Hz, 1H), 7.43 (t, J=4.8 Hz, 1H), 6.43 (d, J=0.9 Hz, 1H), 5.39 (dp, J=4.8, 2.3 Hz, 1H), 4.24-4.14 (m, 1H), 3.85-3.78 (m, 4H), 3.55-3.48 (m, 4H), 2.23-2.14 (m, 2H), 2.01-1.93 (m, 2H), 1.92-1.74 (m, 4H).

m/z calcd. C₂₃H₂₅IN₆O₃=560.1. Found [M+H]⁺=561.2.

### N-[4-[[3-(Methanesulfonamido)-7-morpholino-1,6-naphthyridin-5-yl]oxy]cyclohexyl]pyrimidine-2-carboxamide (LFA015)

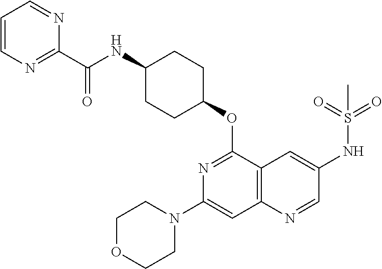 [SMILES: CS(=O)(=O)Nc1cnc2cc(N3CCOCC3)nc(O[C@H]3CC[C@@H](NC(=O)c4ncccn4)CC3)c2c1]

Prepared according to step 6 of Scheme 6 starting with LFA014 and heating for 6 h to afford the titled compound in 67% yield.

¹H NMR (400 MHz, DMSO-d₆) δ 8.96 (d, J=4.9 Hz, 2H), 8.69 (d, J=2.7 Hz, 1H), 8.60 (d, J=7.9 Hz, 1H), 8.13 (dd, J=2.7, 0.8 Hz, 1H), 7.67 (t, J=4.9 Hz, 1H), 6.53 (d, J=0.8 Hz, 1H), 5.41-5.30 (m, 1H), 4.05-3.91 (m, 1H), 3.78-3.70 (m, 4H), 3.53-3.37 (m, 4H), 3.00 (s, 3H), 2.18-2.03 (m, 2H), 1.90-1.73 (m, 6H).

m/z calcd. C₂₄H₂₉N₇O₅S=527.2. Found [M+H]⁺=528.2.

### N-[4-[[3-[(3-Methyl-2-nitro-imidazol-4-yl)methyl-methylsulfonyl-amino]-7-morpholino-1,6-naphthyridin-5-yl]oxy]cyclohexyl]pyrimidine-2-carboxamide (LFA016)

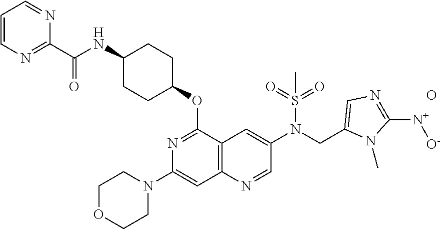

Prepared according to step 7 of Scheme 6 starting with LFA015, LFA007 (1.1 eq), potassium carbonate (2.1 eq) and stirring for 20 h to afford the titled compound in 34% yield.

¹H NMR (400 MHz, DMSO-d₆) δ 8.96 (d, J=4.9 Hz, 2H), 8.71 (d, J=2.6 Hz, 1H), 8.63 (d, J=7.9 Hz, 1H), 8.28 (dd, J=2.6, 0.8 Hz, 1H), 7.68 (t, J=4.9 Hz, 1H), 6.98 (s, 1H), 6.52 (d, J=0.8 Hz, 1H), 5.44-5.36 (m, 1H), 5.17 (s, 2H), 4.00-3.91 (m, 1H), 3.95 (s, 3H), 3.77-3.68 (m, 4H), 3.56-3.47 (m, 4H), 3.26 (s, 3H), 2.13-2.03 (m, 2H), 1.99-1.88 (m, 2H), 1.88-1.72 (m, 4H).

m/z calcd. C₂₉H₃₄N₁₀O₇S=666.2. Found [M+H]⁺=667.2.

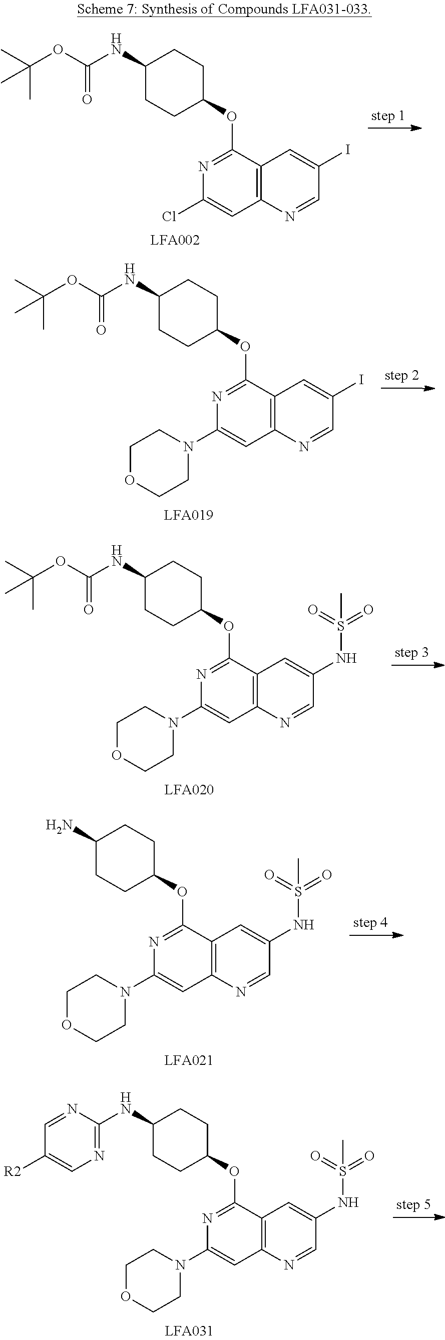

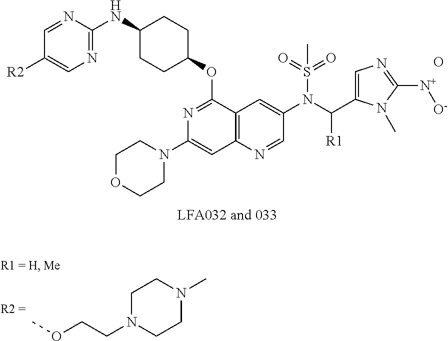

### Step 1

### 5,7-dichloro-3-iodo-1,6-naphthyridine

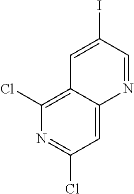 [SMILES: Clc1cc2ncc(I)cc2c(Cl)n1]

N-iodosuccinimide (6.4014 g, 27.884 mmol, 2.86 mL, 1.8500 Eq.) was added to 5,7-dichloro-1,6-naphthyridine (3 g, 15.072 mmol, 1.0000 Eq.) in acetic acid (15.72 g, 261.8 mmol, 15 mL, 17.37 Eq.) and heated to 100° C. for 4 hrs. The reaction mixture was concentrated to a small volume and the residue was diluted with EtOAc. Washed with sat. sodium thiosulphate solution, sodium bicarbonate, water and brine. The organic phase was dried over MgSO4 and concentrated. The residue was purified by column chromatography using Isolera 4 (SiO2, gradient from 1:99 ethyl acetate/petroleum ether to 10% ethyl acetate) to give the product as a white solid 5,7-dichloro-3-iodo-1,6-naphthyridine (2.4 g, 49%).

1H NMR (500 MHz, DMSO-d6) δ 9.40 (d, J=2.29 Hz, 1H), 9.01-9.03 (m, 1H), 8.16-8.17 (m, 1H).

Rt=1.87 min., m/z at 324 M⁺(MeCN, pH1).

Tert-butyl N-[4-[(7-chloro-3-iodo-1,6-naphthyridin-5-yl)oxy]cyclohexyl]carbamate

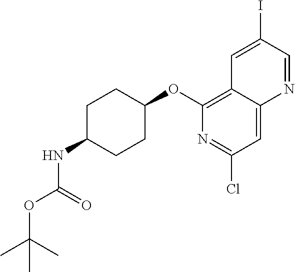 [SMILES: CC(C)(C)OC(=O)N[C@H]1CC[C@@H](Oc2nc(Cl)cc3ncc(I)cc23)CC1]

Tert-butyl cis-4-hydroxycyclohexylcarbamate (1.97 g, 8.86 mmol, 2.00 Eq.) was added to 5,7-dichloro-3-iodo-1,6-naphthyridine (1.44 g, 4.43 mmol, 1.00 Eq.) and cesium carbonate (7.22 g, 22.2 mmol, 1.75 mL, 5.00 Eq.) in 1,4-dioxane (61.92 g, 702.8 mmol, 60 mL, 159 Eq.) and the reaction heated to 110° C. O/N. Water was added followed by EtOAc. The organic phase was washed with brine, dried and concentrated. The residue was purified by column chromatography using Isolera 4 (SiO2, gradient from 1:9 ethyl acetate/petroleum ether to 2:8 ethyl acetate/petroleum ether) to give a white solid tert-butyl N-[4-[(7-chloro-3-iodo-1,6-naphthyridin-5-yl)oxy]cyclohexyl]carbamate (0.285 g, 12.8%).

1H NMR (500 MHz, DMSO-d6) δ 9.25 (d, J=2.29 Hz, 1H), 8.79-8.87 (m, 1H), 7.49-7.57 (m, 1H), 6.81-6.90 (m, 1H), 5.34-5.42 (m, 1H), 1.99-2.08 (m, 2H), 1.60-1.75 (m, 6H), 1.40 (s, 9H).

Rt=2.56 min., m/z at 504 MH+ (MeOH, pH10).

### Tert-butyl N-[4-[(3-iodo-7-morpholino-1,6-naphthyridin-5-yl)oxy]cyclohexyl]carbamate (LFA019)

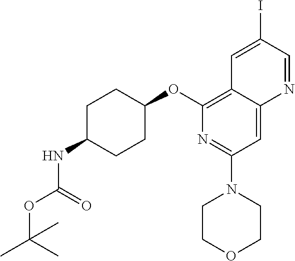 [SMILES: CC(C)(C)OC(=O)N[C@H]1CC[C@@H](Oc2nc(N3CCOCC3)cc3ncc(I)cc23)CC1]

Tert-butyl N-[4-[(7-chloro-3-iodo-1,6-naphthyridin-5-yl)oxy]cyclohexyl]carbamate (0.143 g, 0.2838 mmol) and morpholine (0.999 g, 1 mL, 11.5 mmol) were combined in a microwave vial and the reaction heated to 140° C. at very high absorption for 2 h (note: higher temperature was selected but microwave unable to heat the reaction mixture to a higher temperature than 140° C.). Toluene was added to the reaction mixture, which was then concentrated. Purification by reverse phase column chromatography (Biotage SP4, gradient from 1:9 methanol/water pH10 to 100% methanol) gave the product tert-butyl N-[4-[(3-iodo-7-morpholino-1,6-naphthyridin-5-yl)oxy]cyclohexyl]carbamate (0.135 g, 85.8%) a brown solid.

1H NMR (500 MHz, DMSO-d6) δ 8.88-8.95 (m, 1H), 8.50-8.57 (m, 1H), 6.82-6.89 (m, 1H), 6.43-6.51 (m, 1H), 5.28-5.37 (m, 1H), 3.67-3.75 (m, 4H), 3.46-3.55 (m, 4H), 3.37-3.43 (m, 1H), 2.01-2.07 (m, 2H), 1.59-1.71 (m, 7H), 1.57-1.68 (m, 6H), 1.37-1.41 (m, 9H), 1.37-1.42 (m, 1H).

Rt=1.67 min., m/z at 555 MH+(MeCN, pH1).

### Step 2

### tert-Butyl N-[4-[[3-(methanesulfonamido)-7-morpholino-1,6-naphthyridin-5-yl]oxy]cyclohexyl]carbamate (LFA020)

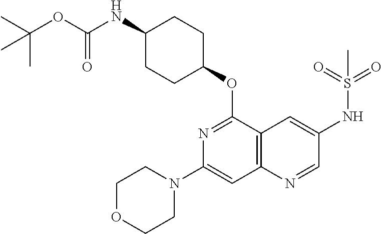 [SMILES: CC(C)(C)OC(=O)N[C@H]1CC[C@@H](Oc2nc(N3CCOCC3)cc3ncc(NS(C)(=O)=O)cc23)CC1]

To a mixture of tert-butyl N-[4-[(3-iodo-7-morpholino-1,6-naphthyridin-5-yl)oxy]cyclohexyl]carbamate LFA019 (2.00 g, 3.61 mmol), methanesulfonamide (0.377 g, 3.97 mmol), copper(I) iodide (0.103 g, 0.54 mmol) and trans-N,N′-dimethylcyclohexane-1,2-diamine (0.17 mL, 1.08 mmol) was added 1,4-dioxane (30 mL) and potassium carbonate (0.997, 7.21 mmol). The mixture was purged with argon and heated at 120° C. (bath temperature) for 19 h. The reaction mixture was filtered through Celite and washed with 5% MeOH/CH₂Cl₂. The combined filtrate and washings were concentrated, and the residue was purified via automated flash chromatography using 40-100% EtOAc/n-heptane as the mobile phase to afford the titled compound as a pale orange glassy solid (1.27 g, 67%).

¹H NMR (400 MHz, CDCl₃) δ 8.76 (s, 1H), 8.30 (s, 1H), 6.56 (s, 1H), 5.46-5.35 (m, 1H), 4.69 (br. s, 1H), 3.91-3.77 (m, 4H), 3.68-3.58 (m, 1H), 3.58-3.51 (m, 4H), 3.07 (s, 3H), 2.17-2.04 (m, 2H), 1.91-1.56 (m, 6H), 1.46 (s, 9H). Sulfonamide NH not directly observed.

m/z calcd. C₂₄H₃₅N₅O₆S=521.2. Found [M+H]⁺=522.2.

### Step 3

### N-[5-(4-Aminocyclohexoxy)-7-morpholino-1,6-naphthyridin-3-yl]methanesulfonamide (LFA021)

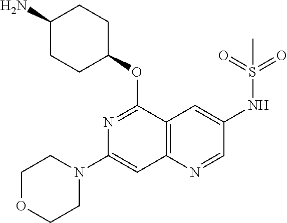 [SMILES: CS(=O)(=O)Nc1cnc2cc(N3CCOCC3)nc(O[C@H]3CC[C@@H](N)CC3)c2c1]

TFA (4.51 mL, 59.0 mmol) was added to a stirred solution of tert-Butyl N-[4-[[3-(methanesulfonamido)-7-morpholino-1,6-naphthyridin-5-yl]oxy]cyclohexyl]carbamate LFA020 (1.03 g, 1.96 mmol) in CH₂Cl₂ (10 mL). The resultant red solution was stirred at room temperature for 2 h and then concentrated. The residue was loaded onto a 10 g SCX cartridge and the cartridge was flushed with MeOH. The product was eluted with 2 M ammonia in MeOH and concentrated to afford the titled compound as a pale orange solid (0.817 g, 99%).

¹H NMR (400 MHz, CDCl₃) δ 8.80 (d, J=2.7 Hz, 1H), 8.26 (dd, J=2.6, 0.8 Hz, 1H), 6.54 (d, J=0.8 Hz, 1H), 5.48-5.40 (m, 1H), 3.88-3.80 (m, 4H), 3.54-3.49 (m, 4H), 3.10-2.98 (m, 1H), 3.03 (s, 3H), 2.20-2.07 (m, 2H), 1.86-1.58 (m, 6H). Sulfonamide NH and NH₂ not directly observed

m/z calcd. C₁₉H₂₇N₅O₄S=421.2. Found [M+H]⁺=422.2.

### Step 4

A microwave vial was charged N-[5-(4-aminocyclohexoxy)-7-morpholino-1,6-naphthyridin-3-yl]methanesulfonamide LFA021, the appropriate aryl chloride (1.5 eq), potassium tert-butoxide (3.0 eq), BrettPhos-Pd-G3 (0.1 eq)) and BrettPhos (0.1 eq). The vial was sealed, and the mixture was purged with a stream of argon for 5 minutes. 1,4-dioxane (0.1 M) was added and the mixture was degassed by sparging with argon for 5 minutes. The mixture was heated to 90° C. and stirred for 16-24 h. The reaction mixture was diluted with EtOAc (2 mL) and filtered through Celite, eluting with EtOAc (30 mL). The filtrate was concentrated to give the crude product. The crude product was purified via automated flash chromatography using (2 M ammonia in MeOH)/CH₂Cl₂ as the mobile phase and/or via automated reverse phase preparative HPLC using 5-95% 0.005 M NH₄OH/MeCN in 0.005 M NH₄OH/H₂O or 5-95% 0.1% HCOOH/MeCN in 0.1% HCOOH/H₂O as the mobile phase to afford compound LFA031.

### Step 5

An RB flask or microwave vial was charged with the appropriate aryl sulfonamide LFA031, alkyl halide LFA007 or LFA009 (1.1-1.65 eq), potassium carbonate (2.1-4.2 eq) and DMF (0.06 M). The suspension was stirred at room temperature under an argon atmosphere for 18-60 h. The mixture was diluted with CH₂Cl₂, filtered through Celite and concentrated. The crude product was purified via automated flash chromatography using EtOAc/n-heptane or MeOH/CH₂Cl₂ as the mobile phase and/or via automated reverse phase preparative HPLC using 5-95% 0.005 M NH₄OH/MeCN in 0.005 M NH₄OH/H₂O or 5-95% 0.1% HCOOH/MeCN in 0.1% HCOOH/H₂O as the mobile phase to afford compounds LFA032 and LFA033.

### 2-Chloro-5-(2-(4-methylpiperazin-1-yl)ethoxy)pyrimidine (43-i)

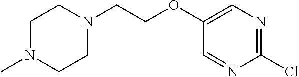 [SMILES: CN1CCN(CCOc2cnc(Cl)nc2)CC1]

Prepared according to general procedure E starting with 2-chloropyrimidin-5-ol and 2-(4-methylpiperazin-1-yl)ethan-1-ol to afford the titled compound in 99% yield. ¹H NMR (400 MHz, DMSO-d₆) δ 8.55 (s, 2H), 4.25 (t, J=5.7 Hz, 2H), 2.70 (t, J=5.8 Hz, 2H), 2.50-2.24 (m, 8H), 2.17 (s, 3H). m/z calcd. for C₁₁H₁₇ClN₄0=256.1. Found [M+H]⁺=257.2.

### N-[5-[4-[[5-[2-(4-Methylpiperazin-1-yl)ethoxy]pyrimidin-2-yl]amino]cyclohexoxy]-7-morpholino-1,6-naphthyridin-3-yl]methanesulfonamide (LFA031)

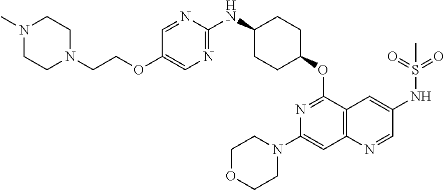 [SMILES: CN1CCN(CCOc2cnc(N[C@H]3CC[C@@H](Oc4nc(N5CCOCC5)cc5ncc(NS(C)(=O)=O)cc45)CC3)nc2)CC1]

Prepared according to step 4b of Scheme 7 starting with LFA021 and 43-i and heating for 18 h to afford the titled compound in 42% yield.

¹H NMR (400 MHz, DMSO-d₆) δ 9.82 (br. s, 1H), 8.68 (d, J=2.6 Hz, 1H), 8.11 (s, 2H), 8.10 (dd, J=2.7, 0.8 Hz, 1H), 6.70 (d, J=7.4 Hz, 1H), 6.52 (d, J=0.9 Hz, 1H), 5.38-5.28 (m, 1H), 4.03 (t, J=5.8 Hz, 2H), 3.83-3.68 (m, 5H), 3.55-3.42 (m, 4H), 3.01 (s, 3H), 2.62 (t, J=5.8 Hz, 2H), 2.47-2.24 (br. m, 8H), 2.14 (s, 3H), 2.12-2.04 (m, 2H), 1.87-1.66 (m, 6H).

m/z calcd. C₃₀H₄₃N₉O₅S=641.3. Found [M+H]⁺=642.2.

### N-[(3-Methyl-2-nitro-imidazol-4-yl)methyl]-N-[5-[4-[[5-[2-(4-methylpiperazin-1-yl)ethoxy]pyrimidin-2-yl]amino]cyclohexoxy]-7-morpholino-1,6-naphthyridin-3-yl]methanesulfonamide (LFA032)

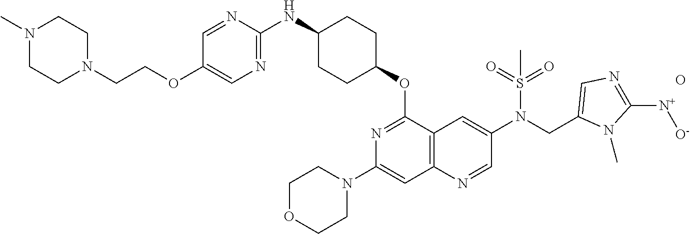

Prepared according to step 5 of Scheme 7 starting with LFA031, LFA007 (1.1 eq), potassium carbonate (2.1 eq) and stirring for 18 h to afford the titled compound in 12% yield.

¹H NMR (400 MHz, DMSO-d₆) δ 8.71 (d, J=2.6 Hz, 1H), 8.18 (dd, J=2.6, 0.8 Hz, 1H), 8.12 (s, 2H), 6.98 (s, 1H), 6.69 (d, J=7.7 Hz, 1H), 6.51 (s, 1H), 5.39-5.34 (m, 1H), 5.15 (s, 2H), 4.04 (t, J=5.7 Hz, 2H), 3.95 (s, 3H), 3.82-3.78 (m, 1H), 3.72 (t, J=4.8 Hz, 4H), 3.54-3.46 (m, 4H), 3.25 (s, 3H), 2.65-2.58 (m, 2H), 2.45-2.21 (br. m, 8H), 2.14 (s, 3H), 2.10-2.01 (m, 2H), 1.83-1.70 (m, 6H).

m/z calcd. C₃₅H₄₈N₁₂O₇S=780.3. Found [M+H]⁺=781.2.

### N-[1-(3-Methyl-2-nitro-imidazol-4-yl)ethyl]-N-[5-[4-[[5-[2-(4-methylpiperazin-1-yl)ethoxy]pyrimidin-2-yl]amino]cyclohexoxy]-7-morpholino-1,6-naphthyridin-3-yl]methanesulfonamide (LFA033)

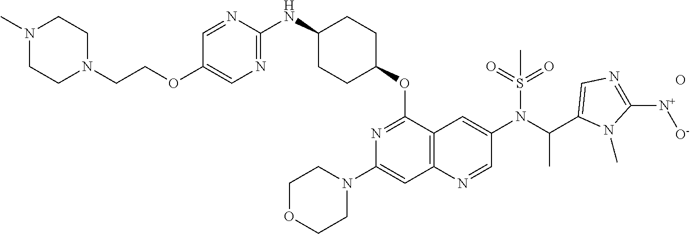

Prepared according to step 5 of Scheme 7 starting with LFA031, LFA009 (1.1 eq), potassium carbonate (2.1 eq) and stirring for 22 h to afford the titled compound in 28% yield.

¹H NMR (400 MHz, CD₃OD) δ 8.36 (d, J=2.6 Hz, 1H), 8.11 (s, 2H), 7.82 (d, J=2.4 Hz, 1H), 6.75 (d, J=0.8 Hz, 1H), 6.46 (d, J=0.9 Hz, 1H), 5.93-5.84 (m, 1H), 5.43-5.34 (m, 1H), 4.28 (s, 3H), 4.12 (t, J=5.4 Hz, 2H), 3.90-3.76 (m, 5H), 3.63-3.54 (m, 4H), 3.20 (s, 3H), 2.79 (t, J=5.4 Hz, 2H), 2.76-2.40 (m, 8H), 2.30 (s, 3H), 2.22-2.06 (m, 2H), 1.97-1.77 (m, 4H), 1.76-1.56 (m, 2H), 1.74 (d, J=6.9 Hz, 3H). NH not directly observed.

m/z calcd. C₃₆H₅₀N₁₂O₇S=794.4. Found [M+H]⁺=795.2.

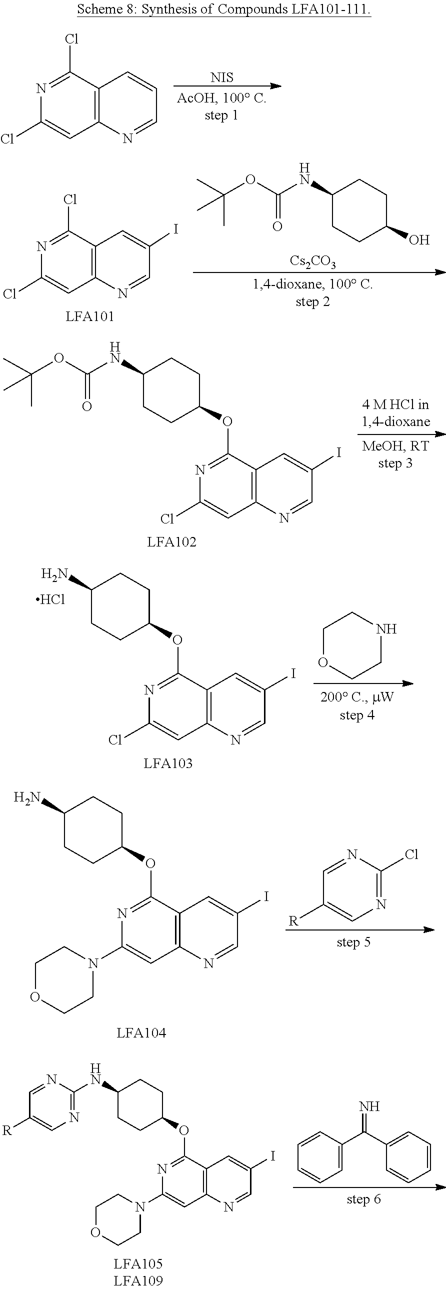

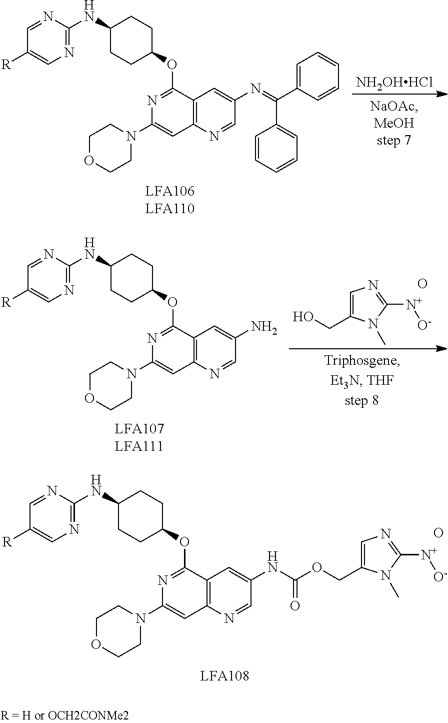

### Step 1

### 5,7-Dichloro-3-iodo-1,6-naphthyridine (LFA101)

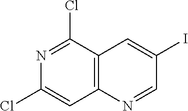 [SMILES: Clc1cc2ncc(I)cc2c(Cl)n1]

5,7-dichloro-1,6-naphthyridine (5.25 g 26.4 mmol) was charged to a 10 mL RB flask, suspended/dissolved in Acetic acid (85 mL) and N-Iodosuccinimide (10.98 g, 48.8 mmol) was added in one portion. The suspension was heated at 100° C. with stirring for 4 h and then cooled to room temperature and concentrated. The residue was dissolved in EtOAc (100 mL) and washed with sat. sodium metabisulphite solution (aq.) (150 mL). The layers were separated, the aqueous extracted with EtOAc (2×50 mL) and the combined organics washed with brine (100 mL), dried over Na₂SO₄, filtered and concentrated. The resulting residue was purified via automated flash chromatography using 0-10% EtOAc in n-heptane as the mobile phase to afford the titled compound as a pale yellow solid (5.23 g, 59%).

¹H NMR (400 MHz, CDCl₃) δ 9.22 (d, J=2.1 Hz, 1H), 8.94 (dd, J=2.0, 1.0 Hz, 1H), 7.89 (d, J=0.9 Hz, 1H).

m/z calcd. for C₈H₃Cl₂IN₂=323.8. Found [M+H]⁺=324.9 (for ³⁵Cl isotope).

### Step 2

### tert-Butyl N-[4-[(7-chloro-3-iodo-1,6-naphthyridin-5-yl)oxy]cyclohexyl]carbamate (LFA102)

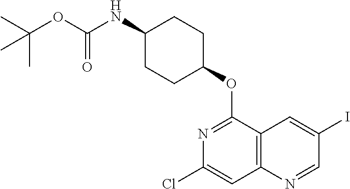 [SMILES: CC(C)(C)OC(=O)N[C@H]1CC[C@@H](Oc2nc(Cl)cc3ncc(I)cc23)CC1]

5,7-dichloro-3-iodo-1,6-naphthyridine (1.00 g, 3.08 mmol), tert-butyl N-(4-hydroxycyclohexyl)carbamate (1.33 g, 6.16 mmol) and cesium carbonate (5.01 g, 15.4 mmol) were charged to a 50 mL RB flask and anhydrous 1,4-dioxane (19 mL) was added. The resultant suspension was heated to 110° C. under an argon atmosphere for 24 h and then cooled to room temperature. The mixture was partitioned between water (100 mL) and EtOAc (3×50 mL) and the combined organics washed with brine (50 mL), dried over Na₂SO₄, filtered and concentrated. The resulting residue was purified via automated flash chromatography using 0-20% EtOAc in n-heptane as the mobile phase to afford the titled compound as a pale yellow solid (1.17 g, 75%).

¹H NMR (400 MHz, CDCl₃) δ 9.12 (d, J=2.2 Hz, 1H), 8.77 (dd, J=2.2, 0.9 Hz, 1H), 7.44 (d, J=0.9 Hz, 1H), 5.49 (qd, J=5.0, 3.0 Hz, 1H), 4.59 (s, 1H), 3.63 (s, 1H), 2.16-2.07 (m, 2H), 1.96-1.76 (m, 4H), 1.70-1.58 (m, 2H), 1.46 (s, 9H).

m/z calcd. for C₁₉H₂₃ClIN₃O₃=503.0. Found [M+H]⁺=504.0 (for ³⁵Cl isotope).

### Step 3

### 4-[(7-Chloro-3-iodo-1,6-naphthyridin-5-yl)oxy]cyclohexanamine; hydrochloride (LFA103)

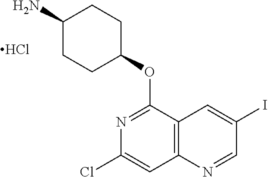 [SMILES: Cl ; N[C@H]1CC[C@@H](Oc2nc(Cl)cc3ncc(I)cc23)CC1]

Hydrochloric acid (3.7-4.3 M in 1,4-dioxane) (16.1 mL, 59.6 mmol) was added to a stirred solution of tert-butyl N-[4-[(7-chloro-3-iodo-1,6-naphthyridin-5-yl)oxy]cyclohexyl]carbamate (3.00 g, 5.96 mmol) in MeOH (30 mL). The mixture was stirred at room temperature for 24 h. The mixture was concentrated and the residue was azeotroped with MeCN (30 mL) and dried in vacuo to afford the titled compound as a yellow solid (3.01 g, 103%).

¹H NMR (400 MHz, DMSO-d₆) δ 9.25 (d, J=2.1 Hz, 1H), 8.89 (dd, J=2.1, 0.9 Hz, 1H), 8.16 (s, 3H), 7.54 (d, J=0.9 Hz, 1H), 5.48-5.39 (m, 1H), 3.23-3.10 (m, 1H), 2.16-2.09 (m, 2H), 1.93-1.69 (m, 6H).

m/z calcd. for C₁₄H₁₅ClIN₃O=403.0. Found [M+H]⁺=404.0 (for ³⁵Cl isotope).

### Step 4

### 4-[(3-Iodo-7-morpholino-1,6-naphthyridin-5-yl)oxy]cyclohexanamine (LFA104)

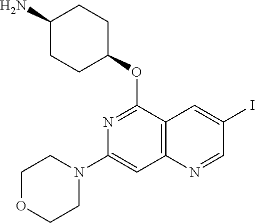 [SMILES: N[C@H]1CC[C@@H](Oc2nc(N3CCOCC3)cc3ncc(I)cc23)CC1]

The material was split into three equal batches of 0.871 g of starting material and 12 mL morpholine and heated in the microwave separately. The batches were then combined for work-up and purification. The procedure below is written as if the reaction was performed in a single batch.

A mixture of 4-[(7-chloro-3-iodo-1,6-naphthyridin-5-yl)oxy]cyclohexanamine; hydrochloride (2.62 g, 5.96 mmol) and morpholine (36.0 mL, 417 mmol) was heated in the microwave at 200° C. for 1 h. The mixture was concentrated and most of the residual morpholine was removed by azeotroping with toluene (2×20 mL). The crude product was purified via automated flash chromatography using 0-10% 2 M NH₃/MeOH in CH₂Cl₂ as the mobile phase to afford an orange solid (2.11 g). The solid was suspended in TBME (30 mL) and stirred vigorously for 30 minutes. The suspension was filtered in vacuo and the collected solid was washed with TBME (2×30 mL) and dried to give the titled compound as a yellow solid (1.87 g, 69%).

¹H NMR (400 MHz, DMSO-d₆) δ 8.89 (d, J=2.2 Hz, 1H), 8.52 (d, J=2.2 Hz, 1H), 6.48 (s, 1H), 5.44-5.21 (m, 1H), 3.76-3.66 (m, 4H), 3.54-3.45 (m, 4H), 2.75 (tt, J=8.9, 3.7 Hz, 1H), 2.11-1.96 (m, 2H), 1.73-1.57 (m, 4H), 1.57-1.41 (m, 2H). NH₂ not directly observed.

m/z calcd. for C₁₈H₂₃IN₄O₂=454.1. Found [M+H]⁺=455.1.

### Step 5

A microwave vial was charged with 4-[(3-iodo-7-morpholino-1,6-naphthyridin-5-yl)oxy]cyclohexanamine LFA104, the appropriate pyrimidine chloride (1.5-2.5 eq), triethylamine (2.0-3.0 eq) and EtOH (O2-0.25 M). The vial was sealed, placed in a microwave reactor and eradiated with microwaves for 2-20 h at 150° C. The mixture was concentrated and partitioned between sat. NaHCO₃ (aq.) and CH₂Cl₂ (3×). The combined organics were dried by passing through a hydrophobic frit and concentrated. The crude product was purified via automated flash chromatography using EtOAc/n-heptane or MeOH/CH₂Cl₂ as the mobile phase to give the compounds LFA105 and LFA109.

### Step 6

A mixture of the appropriate iodide, benzophenone imine (1.2-1.5 eq), tris(dibenzylideneacetone)dipalladium(0) (0.1 eq), 9,9-dimethyl-4,5-bis(diphenylphosphino)xanthene (0.2 eq) and cesium carbonate (1.5 eq) was purged with a stream of argon for 5 minutes. 1,4-dioxane (0.1 M) was added and the mixture was degassed by sparging with argon for 5 minutes. The mixture was heated to 100° C. and stirred for 4-20 h. On cooling the mixture was diluted with EtOAc, filtered through a pad of Celite and washed with EtOAc. The combined filtrate and washings was concentrated and the residue was purified via automated flash chromatography using 0-8% 2 M NH₃/MeOH in CH₂Cl₂ as the mobile phase. Further purification was achieved using a SCX column to give the compounds LFA106 and LFA110.

### Step 7

Hydroxylamine hydrochloride (1.8 eq) and sodium acetate (2.4 eq) were added to a stirred suspension of the appropriate diphenyl imine in MeOH (0.1 M). The mixture was stirred at room temperature for 2.5-3 h. The solvent was removed in vacuo and the residue was partitioned between sat. NaHCO₃ (aq.) and CH₂Cl₂. The layers were separated, and the aqueous phase was extracted with CH₂Cl₂. The combined organic phases were dried (phase-separator) and concentrated in vacuo. The crude product was purified via automated flash chromatography using 0-10% 2 M NH₃/MeOH in CH₂Cl₂ as the mobile phase or by preparative HPLC using 5-95% 0.005 M NH₄OH/MeCN in 0.005 M NH₄OH/H₂O as the mobile phase to give the compounds LFA107 and LFA111.

### N-[4-[(3-Iodo-7-morpholino-1,6-naphthyridin-5-yl)oxy]cyclohexyl]pyrimidin-2-amine (LFA105)

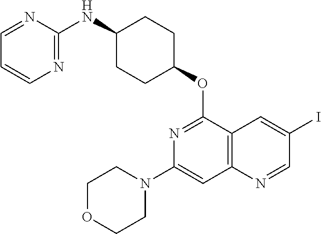 [SMILES: Ic1cnc2cc(N3CCOCC3)nc(O[C@H]3CC[C@@H](Nc4ncccn4)CC3)c2c1]

Prepared according to step 5 of Scheme 8 starting with 2-chloropyrimidine (2.5 eq), triethylamine (3.0 eq) and heating for 20 h to afford the titled compound in 78% yield.

¹H NMR (400 MHz, CDCl₃) δ 8.89 (d, J=2.3 Hz, 1H), 8.59 (dd, J=2.2, 0.8 Hz, 1H), 8.29 (d, J=4.8 Hz, 2H), 6.53 (t, J=4.8 Hz, 1H), 6.47 (d, J=0.9 Hz, 1H), 5.44-5.38 (m, 1H), 5.22 (d, J=8.0 Hz, 1H), 4.06-3.93 (m, 1H), 3.88-3.79 (m, 4H), 3.58-3.50 (m, 4H), 2.22-2.10 (m, 2H), 2.05-1.95 (m, 2H), 1.94-1.64 (m, 4H).

m/z calcd. for C₂₂H₂₅IN₆O₂=532.1. Found [M+H]⁺=533.1.

### N-[4-[[3-(Benzhydrylideneamino)-7-morpholino-1,6-naphthyridin-5-yl]oxy]cyclohexyl]pyrimidin-2-amine (LFA106)

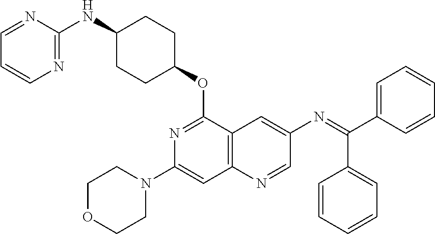 [SMILES: c1ccc(C(=Nc2cnc3cc(N4CCOCC4)nc(O[C@H]4CC[C@@H](Nc5ncccn5)CC4)c3c2)c2ccccc2)cc1]

Prepared according to step 6 of Scheme 8 starting with N-[4-[(3-Iodo-7-morpholino-1,6-naphthyridin-5-yl)oxy]cyclohexyl]pyrimidin-2-amine LFA105, benzophenone imine (1.5 eq) and heating for 20 h to afford the crude titled compound in 45% yield.

¹H NMR (400 MHz, CDCl₃) δ 8.37 (d, J=2.6 Hz, 1H), 8.27 (d, J=4.8 Hz, 2H), 7.81-7.79 (m, 1H), 7.79-7.77 (m, 1H), 7.64 (dd, J=2.6, 0.8 Hz, 1H), 7.52-7.47 (m, 1H), 7.46-7.39 (m, 2H), 7.34-7.27 (m, 3H), 7.21-7.15 (m, 2H), 6.51 (t, J=4.8 Hz, 1H), 6.46 (d, J=0.9 Hz, 1H), 5.39-5.33 (m, 1H), 5.14 (d, J=8.1 Hz, 1H), 3.93 (dddd, J=14.3, 10.3, 8.3, 4.3 Hz, 1H), 3.89-3.78 (m, 4H), 3.49 (d, J=9.5 Hz, 4H), 2.17-2.06 (m, 2H), 2.03-1.88 (m, 2H), 1.84-1.71 (m, 2H), 1.67-1.53 (m, 2H). ca. 75% purity (ca. 25 wt % SM present).

m/z calcd. for C₃₅H₃₅N₇O₂=585.7. Found [M+H]⁺=586.3.

### 7-morpholino-5-[4-(pyrimidin-2-ylamino)cyclohexoxy]-1,6-naphthyridin-3-amine (LFA107)

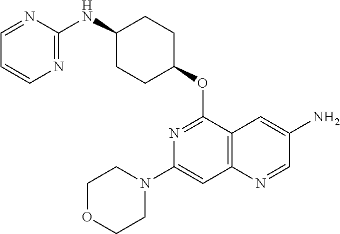 [SMILES: Nc1cnc2cc(N3CCOCC3)nc(O[C@H]3CC[C@@H](Nc4ncccn4)CC3)c2c1]

Prepared according to step 7 of Scheme 8 starting with N-[4-[[3-(benzhydrylideneamino)-7-morpholino-1,6-naphthyridin-5-yl]oxy]cyclohexyl]pyrimidin-2-amine, hydroxylamine hydrochloride (1.8 eq) and sodium acetate (2.4 eq) in MeOH and stirring at room temperature for 2.5 h to afford the titled compound as a yellow solid in 79% yield.

¹H NMR (400 MHz, DMSO-d₆) δ 8.42 (d, J=2.8 Hz, 1H), 8.26 (d, J=4.7 Hz, 2H), 7.38 (dd, J=2.9, 0.8 Hz, 1H), 7.05 (d, J=7.5 Hz, 1H), 6.53 (t, J=4.8 Hz, 1H), 6.40 (d, J=0.9 Hz, 1H), 5.41 (s, 2H), 5.32-5.25 (m, 1H), 3.92-3.80 (m, 1H), 3.78-3.68 (m, 4H), 3.36-3.32 (m, 4H), 2.15-2.02 (m, 2H), 1.87-1.66 (m, 6H).

m/z calcd. for C₂₂H₂₇N₇O₂=421.5. Found [M+H]⁺=422.2.

### (3-methyl-2-nitro-imidazol-4-yl)methyl N-[7-morpholino-5-[4-(pyrimidin-2-ylamino)cyclohexoxy]-1,6-naphthyridin-3-yl]carbamate (LFA108)

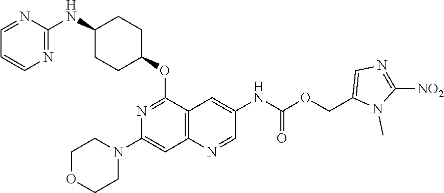 [SMILES: Cn1c(COC(=O)Nc2cnc3cc(N4CCOCC4)nc(O[C@H]4CC[C@@H](Nc5ncccn5)CC4)c3c2)cnc1[N+](=O)[O-]]

7-morpholino-5-[4-(pyrimidin-2-ylamino)cyclohexoxy]-1,6-naphthyridin-3-amine (57 mg, 0.14 mmol) and triethylamine (0.04 mL, 0.300 mmol) were added in THF (4 mL) to a stirred solution of bis(trichloromethyl)carbonate (26 mg, 0.09 mmol) in THF (4 mL). The mixture was stirred at room temperature for 30 min. (3-methyl-2-nitro-imidazol-4-yl)methanol (34 mg, 0.22 mmol) and triethylamine (0.04 mL, 0.300 mmol) were added in THF (2 mL) and the mixture stirred at room temperature for 20 h. The mixture was concentrated and the residue partitioned between CH₂Cl₂ (20 mL) and sat. NaHCO₃ (aq.) (20 mL). The layers were separated, and the combined organic phases were dried (phase-separator). The residue was purified via automated flash chromatography using 0-8% MeOH/CH₂Cl₂ as the mobile phase and then further purified by preparative HPLC using 5-95% 0.005 M NH₄OH/MeCN in 0.005 M NH₄OH/H₂O as the mobile phase to afford the titled compound as a yellow solid (7 mg, 9%).

¹H NMR (400 MHz, DMSO-d₆) δ 10.15 (s, 1H), 8.81 (d, J=2.7 Hz, 1H), 8.50 (s, 1H), 8.27 (d, J=4.8 Hz, 2H), 7.33 (s, 1H), 7.08 (d, J=7.3 Hz, 1H), 6.54 (t, J=4.7 Hz, 1H), 6.51 (d, J=0.9 Hz, 1H), 5.35-5.29 (m, 1H), 5.34 (s, 2H), 3.98 (s, 3H), 3.91-3.79 (m, 1H), 3.77-3.68 (m, 4H), 3.53-3.39 (m, 4H), 2.18-2.08 (m, 2H), 1.90-1.63 (m, 6H).

m/z calcd. for C₂₈H₃₂N₁₀O₆=604.6. Found [M+H]⁺=605.2.

### 2-((2-Chloropyrimidin-5-yl)oxy)-N,N-dimethylacetamide (7-i)

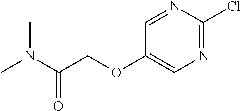

Prepared according to general procedure D starting with 2-chloropyrimidin-5-ol and 2-chloro-N,N-dimethylacetamide to afford the titled compound in 97% yield. ¹H NMR (400 MHz, DMSO-d₆) δ 8.50 (s, 2H), 5.08 (s, 2H), 2.96 (s, 3H), 2.85 (s, 3H). m/z calcd. for C₈H₁₀ClN₃O₂=215.1. Found [M+H]⁺=216.1.

### 2-[2-[[4-[(3-Iodo-7-morpholino-1,6-naphthyridin-5-yl)oxy]cyclohexyl]amino]pyrimidin-5-yl]oxy-N,N-dimethyl-acetamide (LFA109)

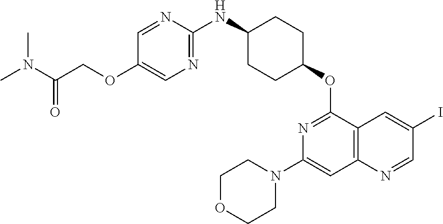 [SMILES: CN(C)C(=O)COc1cnc(N[C@H]2CC[C@@H](Oc3nc(N4CCOCC4)cc4ncc(I)cc34)CC2)nc1]

Prepared according to step 5 of Scheme 8 starting with 2-(2-chloropyrimidin-5-yl)oxy-N,N-dimethyl-acetamide 7i (1.5 eq), triethylamine (2.0 eq) and heating for 12 h to afford the titled compound in 31% yield.

¹H NMR (400 MHz, DMSO-d₆) δ 8.91 (d, J=2.2 Hz, 1H), 8.58 (dd, J=2.3, 0.8 Hz, 1H), 8.08 (s, 2H), 6.81 (d, J=8.0 Hz, 1H), 6.49 (d, J=0.9 Hz, 1H), 5.42-5.33 (m, 1H), 4.77 (s, 2H), 3.85-3.76 (m, 1H), 3.76-3.68 (m, 4H), 3.55-3.46 (m, 4H), 2.95 (s, 3H), 2.83 (s, 3H), 2.15-2.02 (m, 2H), 1.84-1.64 (m, 6H).

m/z calcd. for C₂₆H₃₂IN₇O₄=633.5. Found [M+H]⁺=634.2

### 2-[2-[[4-[[3-(Benzhydrylideneamino)-7-morpholino-1,6-naphthyridin-5-yl]oxy]cyclohexyl]amino]pyrimidin-5-yl]oxy-N,N-dimethyl-acetamide (LFA110)

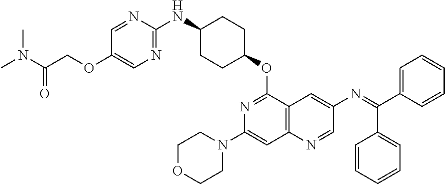 [SMILES: CN(C)C(=O)COc1cnc(N[C@H]2CC[C@@H](Oc3nc(N4CCOCC4)cc4ncc(N=C(c5ccccc5)c5ccccc5)cc34)CC2)nc1]

Prepared according to step 6 of Scheme 8 starting with 2-[2-[[4-[(3-iodo-7-morpholino-1,6-naphthyridin-5-yl)oxy]cyclohexyl]amino]pyrimidin-5-yl]oxy-N,N-dimethyl-acetamide LFA109, benzophenone imine (1.2 eq) and heating for 4 h to afford the crude titled compound in 83% yield. The crude product was used in the next stage without further purification.

m/z calcd. for C₃₉H₄₂N₈O₄=686.8. Found [M+H]⁺=687.2. Note—product breaks down under acidic LC-MS conditions to partially liberate the amine.

### 2-[2-[[4-[(3-Amino-7-morpholino-1,6-naphthyridin-5-yl)oxy]cyclohexyl]amino]pyrimidin-5-yl]oxy-N,N-dimethyl-acetamide (LFA111)

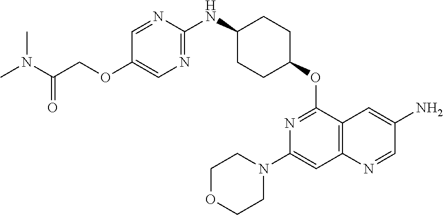 [SMILES: CN(C)C(=O)COc1cnc(N[C@H]2CC[C@@H](Oc3nc(N4CCOCC4)cc4ncc(N)cc34)CC2)nc1]

Prepared according to step 7 of Scheme 8 starting with 2-[2-[[4-[[3-(benzhydrylideneamino)-7-morpholino-1,6-naphthyridin-5-yl]oxy]cyclohexyl]amino]pyrimidin-5-yl]oxy-N,N-dimethyl-acetamide LFA110 to afford the titled compound as a yellow solid in 40% yield.

¹H NMR (400 MHz, DMSO-d₆) δ 8.41 (d, J=2.8 Hz, 1H), 8.09 (s, 2H), 7.37 (dd, J=2.9, 0.9 Hz, 1H), 6.71 (d, J=7.4 Hz, 1H), 6.40 (d, J=0.8 Hz, 1H), 5.41 (s, 2H), 5.30-5.26 (m, 1H), 4.77 (s, 2H), 3.80-3.69 (m, 5H), 3.37-3.32 (m, 4H), 2.95 (s, 3H), 2.83 (s, 3H), 2.08 (s, 2H), 1.85-1.67 (m, 6H).

m/z calcd. for C₂₆H₃₄N₈O₄=522.6. Found [M+H]⁺=523.3

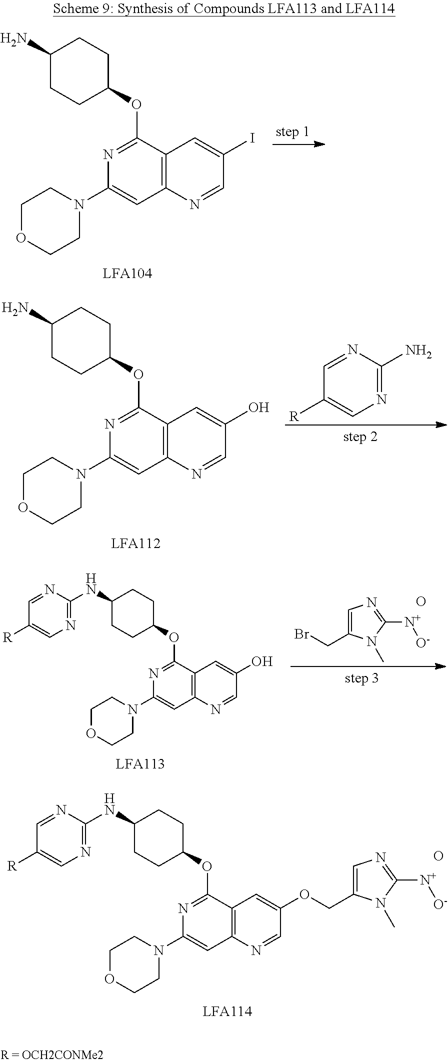

### Step 1

### 5-(4-Aminocyclohexoxy)-7-morpholino-1,6-naphthyridin-3-ol (LFA112)

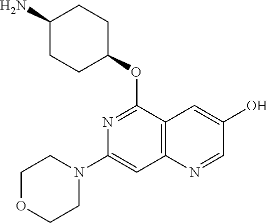 [SMILES: N[C@H]1CC[C@@H](Oc2nc(N3CCOCC3)cc3ncc(O)cc23)CC1]

7.91 M Potassium hydroxide (aq.) (0.23 mL, 1.85 mmol) was added to a mixture of 4-[(3-iodo-7-morpholino-1,6-naphthyridin-5-yl)oxy]cyclohexanamine LFA104 (140 mg, 0.31 mmol), t-BuBrettPhos-Pd-G3 (13 mg, 0.02 mmol) and t-BuBrettPhos (8 mg, 0.02 mmol) in 1,4-dioxane (4.0 mL) The mixture was sparged with argon for 5 minutes and heated to 90° C. under microwave irradiation for 1.5 h. The mixture was diluted with 10% AcOH in MeOH (5 mL), loaded directly onto an SCX column and flushed with MeOH (200 mL) followed by 2 M NH₃/MeOH (100 mL). The NH₃/MeOH fraction was concentrated in vacuo to give the crude titled compound as a yellow/orange gum (116 mg, 98%).

¹H NMR (400 MHz, Methanol-d₄) δ 8.42 (d, J=2.9 Hz, 1H), 7.59 (dd, J=3.0, 0.8 Hz, 1H), 6.42 (d, J=0.9 Hz, 1H), 5.44-5.38 (m, 1H), 3.86-3.77 (m, 4H), 3.45-3.37 (m, 4H), 3.05-2.96 (m, 1H), 2.25-2.14 (m, 2H), 1.89-1.66 (m, 6H). OH and NH₂ not observed.

m/z calcd. for C₁₈H₂₄N₄O₃=344.4. Found [M+H]⁺=345.2

### Step 2

### 2-[2-[[4-[(3-Hydroxy-7-morpholino-1,6-naphthyridin-5-yl)oxy]cyclohexyl]amino]pyrimidin-5-yl]oxy-N,N-dimethyl-acetamide (LFA113)

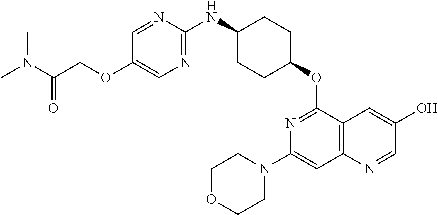 [SMILES: CN(C)C(=O)COc1cnc(N[C@H]2CC[C@@H](Oc3nc(N4CCOCC4)cc4ncc(O)cc34)CC2)nc1]

A mixture of 5-(4-aminocyclohexoxy)-7-morpholino-1,6-naphthyridin-3-ol LFA112 (108 mg, 0.31 mmol), 2-(2-chloropyrimidin-5-yl)oxy-N,N-dimethyl-acetamide (135 mg, 0.63 mmol) and DIPEA (0.16 mL, 0.94 mmol) in n-BuOH (3.0 mL) was stirred at 150° C. under microwave irradiation for 16.5 h. The mixture was diluted with 5% AcOH/MeOH (2 mL) loaded directly onto an SCX column and flushed with MeOH (150 mL) followed by 2 M NH₃/MeOH (100 mL). The NH₃/MeOH fraction was concentrated and the residue purified via automated flash chromatography using 0-20% 2 M NH₃/MeOH in CH₂Cl₂ as the mobile phase to give the crude product. The crude product was further purified twice via preparative HPLC using 5-95% 0.1% HCOOH/MeCN in 0.1% HCOOH/H₂O as the mobile phase followed by using 5-95% 0.005 M NH₄OH/MeCN in 0.005 M NH₄OH/H₂O) as the mobile phase to afford the titled compound as a yellow solid (17 mg, 10%).

¹H NMR (400 MHz, DMSO-d₆) δ 9.98 (s, 1H), 8.53 (d, J=2.9 Hz, 1H), 8.09 (s, 2H), 7.62 (dd, J=2.9, 0.8 Hz, 1H), 6.75 (d, J=7.5 Hz, 1H), 6.48 (d, J=0.9 Hz, 1H), 5.40-5.29 (m, 1H), 4.76 (s, 2H), 3.84-3.70 (m, 5H), 3.45-3.35 (m, 4H), 2.95 (s, 3H), 2.83 (s, 3H), 2.19-2.01 (m, 2H), 1.89-1.64 (m, 6H).

m/z calcd. for C₂₆H₃₃N₇O₅=523.6. Found [M+H]⁺=524.3

### 5-(Bromomethyl)-1-methyl-2-nitro-imidazole (LFA007)

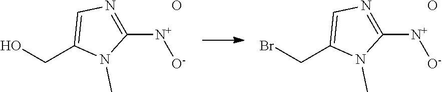

Dibromo(triphenyl)-λ\hat{ }5-phosphane (2.36 g, 5.60 mmol) was added as a suspension in CH₂Cl₂ (10 mL) to an ice-cooled stirred suspension of (3-methyl-2-nitro-imidazol-4-yl)methanol (0.80 g, 5.09 mmol) and DIPEA (1.77 mL, 10.2 mmol) in CH₂Cl₂ (15 mL). The mixture was stirred at 0-5° C. for 2 h, then allowed to warm to room temperature and stirred for 22 h. The mixture was adsorbed directly onto silica gel and was purified via automated flash chromatography using 0-60% EtOAc/n-heptane as the mobile phase to afford the titled compound as a pale yellow solid (0.71 g, 63%).

¹H NMR (400 MHz, CDCl₃) δ 7.20 (d, J=0.5 Hz, 1H), 4.47 (d, J=0.6 Hz, 2H), 4.04 (s, 3H).

m/z calcd. for C₅H₆BrN₃O₂=219.0. Found [M+H]⁺=220.1 (for ⁷⁹Br isotope).

### Step 3

### N,N-Dimethyl-2-[2-[[4-[[3-[(3-methyl-2-nitro-imidazol-4-yl)methoxy]-7-morpholino-1,6-naphthyridin-5-yl]oxy]cyclohexyl]amino]pyrimidin-5-yl]oxy-acetamide (LFA114)

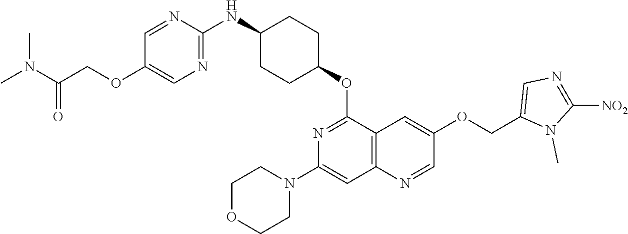 [SMILES: CN(C)C(=O)COc1cnc(N[C@H]2CC[C@@H](Oc3nc(N4CCOCC4)cc4ncc(OCc5cnc([N+](=O)[O-])n5C)cc34)CC2)nc1]

A mixture of 2-[2-[[4-[(3-hydroxy-7-morpholino-1,6-naphthyridin-5-yl)oxy]cyclohexyl]amino]pyrimidin-5-yl]oxy-N,N-dimethyl-acetamide LFA113 (19 mg, 0.04 mmol), 5-(bromomethyl)-1-methyl-2-nitro-imidazole (9 mg, 0.04 mmol) and potassium carbonate (16 mg, 0.11 mmol) was purged with argon for 5 minutes. DMF (1 mL) was added and the mixture was stirred at room temperature for 22 h. A further portion of 5-(bromomethyl)-1-methyl-2-nitro-imidazole (4 mg, 0.020 mmol) was added and the mixture stirred at room temperature for 3 h. The mixture was diluted with 5% AcOH/MeOH (2 mL) and was loaded directly onto an SCX column and flushed with MeOH (100 mL) followed by 2 M NH₃/MeOH (80 mL). The NH₃/MeOH fraction was concentrated in vacuo to give the crude product. The crude product was purified via preparative. HPLC using 5-95% 0.005 M NH₄OH/MeCN in 0.005 M NH₄OH/H₂O as the mobile phase to afford the titled compound as a yellow solid (7 mg, 30%)

¹H NMR (400 MHz, DMSO-d₆) δ 8.69 (d, J=3.0 Hz, 1H), 8.09 (s, 2H), 7.84 (dd, J=3.1, 0.8 Hz, 1H), 7.39 (s, 1H), 6.72 (d, J=7.9 Hz, 1H), 6.54 (d, J=0.8 Hz, 1H), 5.44 (s, 2H), 5.43-5.38 (m, 1H), 4.77 (s, 2H), 4.01 (s, 3H), 3.88-3.77 (m, 1H), 3.77-3.70 (m, 4H), 3.47-3.40 (m, 4H), 2.95 (s, 3H), 2.83 (s, 3H), 2.16-2.02 (m, 2H), 1.85-1.72 (m, 6H).

m/z calcd. for C₃₁H₃₈N₁₀O₇=662.7. Found [M+H]⁺=663.2

### N-[4-[(3-Hydroxy-7-morpholino-1,6-naphthyridin-5-yl)oxy]cyclohexyl]pyrimidine-2-carboxamide (LFA115)

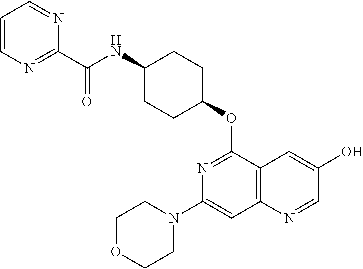 [SMILES: O=C(N[C@H]1CC[C@@H](Oc2nc(N3CCOCC3)cc3ncc(O)cc23)CC1)c1ncccn1]

1-Propanephosphonic anhydride (50% wt in EtOAc) (0.19 mL, 0.330 mmol) was added to a stirred solution of 5-(4-aminocyclohexoxy)-7-morpholino-1,6-naphthyridin-3-ol LFA112 (75 mg, 0.22 mmol), pyrimidine-2-carboxylic acid (32 mg, 0.26 mmol) and DIPEA (0.11 mL, 0.65 mmol) in CH₂Cl₂ (2.5 mL). The mixture was stirred at room temperature for 21 h, concentrated and the residue was re-dissolved in MeOH (3 mL) and 2 M sodium hydroxide (aq.) (1.00 mL, 2.00 mmol) was added. The mixture was stirred at room temperature for 21 h. The mixture was neutralised to ˜pH 6-7 with 1 M HCl (aq.) and further diluted with water (20 mL). The aqueous phase was extracted with EtOAc (3×20 mL) and the combined organic phases were dried via a hydrophobic frit and concentrated. The residue was purified via preparative HPLC using 5-95% 0.005 M NH₄OH/MeCN in 0.005 M NH₄OH/H₂O as the mobile phase to give the titled compound as a bright yellow solid (22 mg, 22%).

¹H NMR (400 MHz, Methanol-d₄) δ 8.94 (d, J=4.9 Hz, 2H), 8.46 (d, J=2.9 Hz, 1H), 7.80 (dd, J=3.0, 0.9 Hz, 1H), 7.62 (t, J=4.9 Hz, 1H), 6.45 (d, J=0.9 Hz, 1H), 5.52-5.40 (m, 1H), 4.08 (tt, J=9.9, 4.4 Hz, 1H), 3.88-3.74 (m, 4H), 3.55-3.41 (m, 4H), 2.34-2.16 (m, 2H), 2.05-1.82 (m, 6H). OH and NH not directly observed.

m/z calcd. For C₂₃H₂₆N₆O₄=450.5. Found [M+H]⁺=451.2

### N-[4-[[3-[(3-Methyl-2-nitro-imidazol-4-yl)methoxy]-7-morpholino-1,6-naphthyridin-5-yl]oxy]cyclohexyl]pyrimidine-2-carboxamide (LFA116)

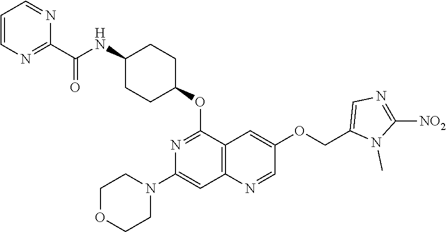 [SMILES: Cn1c(COc2cnc3cc(N4CCOCC4)nc(O[C@H]4CC[C@@H](NC(=O)c5ncccn5)CC4)c3c2)cnc1[N+](=O)[O-]]

A mixture of N-[4-[(3-hydroxy-7-morpholino-1,6-naphthyridin-5-yl)oxy]cyclohexyl]pyrimidine-2-carboxamide LFA115 (17 mg, 0.04 mmol), 5-(bromomethyl)-1-methyl-2-nitro-imidazole (9 mg, 0.04 mmol) and potassium carbonate (11 mg, 0.08 mmol) in DMF (1.3 mL) was stirred at room temperature for 22 h. The mixture was diluted with EtOAc (20 mL) and washed with water (20 mL). The aqueous phase was extracted with EtOAc (2×20 mL) and the combined organic phases were washed with brine (50 mL), dried via a hydrophobic frit and concentrated. The residue was purified via preparative HPLC using 5-95% 0.005 M NH₄OH/MeCN in 0.005 M NH₄OH/H₂O as the mobile phase to afford the titled compound as a bright yellow solid (11 mg, 50%).

¹H NMR (400 MHz, DMSO-d₆) δ 8.95 (d, J=4.9 Hz, 2H), 8.70 (d, J=3.0 Hz, 1H), 8.67 (d, J=8.1 Hz, 1H), 7.92 (dd, J=3.1, 0.8 Hz, 1H), 7.67 (t, J=4.9 Hz, 1H), 7.39 (s, 1H), 6.55 (d, J=0.9 Hz, 1H), 5.47 (s, 2H), 5.45-5.39 (m, 1H), 4.00 (s, 3H), 4.05-3.90 (m, 1H), 3.78-3.70 (m, 4H), 3.49-3.41 (m, 4H), 2.18-2.06 (m, 2H), 2.05-1.93 (m, 2H), 1.90-1.71 (m, 4H).

m/z calcd. For C₂₈H₃₁N₉O₆=589.6. Found [M+H]⁺=590.2

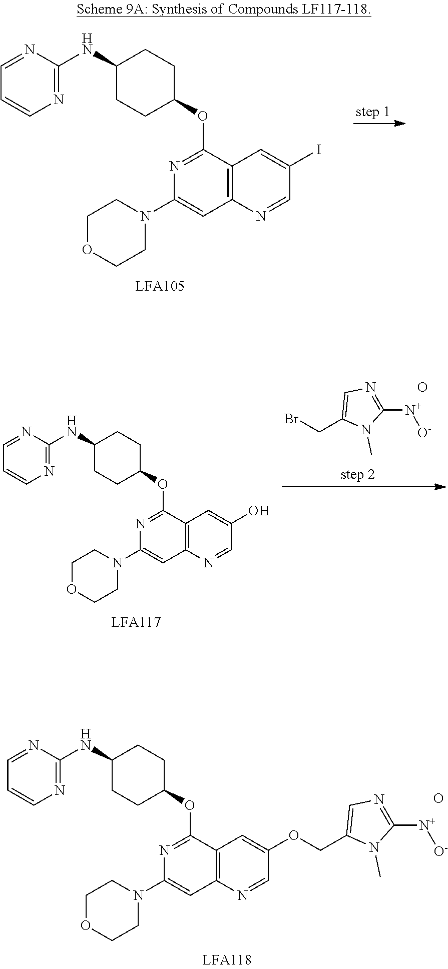

### Step 1

### 7-Morpholino-5-[4-(pyrimidin-2-ylamino)cyclohexoxy]-1,6-naphthyridin-3-ol (LFA117)

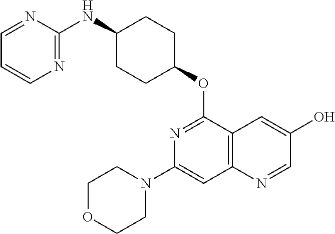 [SMILES: Oc1cnc2cc(N3CCOCC3)nc(O[C@H]3CC[C@@H](Nc4ncccn4)CC3)c2c1]

7.91 M potassium hydroxide (aq.) (0.07 mL, 0.56 mmol) was added to a mixture of N-[4-[(3-iodo-7-morpholino-1,6-naphthyridin-5-yl)oxy]cyclohexyl]pyrimidin-2-amine LFA105 (50 mg, 0.09 mmol), t-BuBrettPhos-Pd-G3 (4 mg, 0.005) and t-BuBrettPhos (2 mg, 0.005 mmol) in 1,4-dioxane (2.5 mL). The mixture was sparged with argon for 5 minutes and heated to 90° C. under microwave irradiation and stirred for 1 h. The mixture was diluted with EtOAc (5 mL), filtered through a pad of Celite, eluting with 10% MeOH in EtOAc (50 mL). The filtrate was concentrated and the residue was purified via automated flash chromatography using 0.5-8% MeOH/CH₂Cl₂ as the mobile phase to give the crude product. The crude product was further purified twice via preparative HPLC using 5-95% 0.005 M NH₄OH/MeCN in 0.005 M NH₄OH/H₂O as the mobile and using 5-95% 0.005 M TFA/MeCN in 0.005 M TFA/H₂O as the mobile phase to afford the product as the TFA salt. The TFA salt of the product was loaded onto an SCX column and the column was flushed with MeOH (120 mL) followed by 2 M NH₃/MeOH (70 mL). The NH₃/MeOH fraction was concentrated to afford the titled compound as a bright yellow solid (17 mg, 43%).

¹H NMR (400 MHz, DMSO-d₆) δ 8.54 (d, J=2.9 Hz, 1H), 8.26 (d, J=4.7 Hz, 2H), 7.63 (dd, J=2.9, 0.7 Hz, 1H), 7.10 (d, J=7.7 Hz, 1H), 6.53 (t, J=4.7 Hz, 1H), 6.48 (d, J=0.9 Hz, 1H), 5.39-5.29 (m, 1H), 3.92-3.80 (m, 1H), 3.80-3.64 (m, 4H), 3.44-3.35 (m, 4H), 2.17-2.00 (m, 2H), 1.87-1.68 (m, 6H). OH not directly observed.

m/z calcd. For C₂₂H₂₆N₆O₃=422.5. Found [M+H]⁺=423.2

### Step 2

### N-[4-[[3-[(3-Methyl-2-nitro-imidazol-4-yl)methoxy]-7-morpholino-1,6-naphthyridin-5-yl]oxy]cyclohexyl]pyrimidin-2-amine (LFA118)

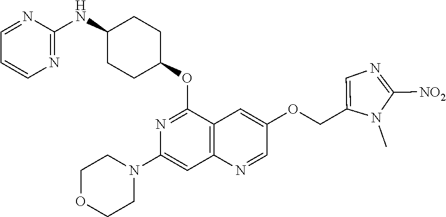 [SMILES: Cn1c(COc2cnc3cc(N4CCOCC4)nc(O[C@H]4CC[C@@H](Nc5ncccn5)CC4)c3c2)cnc1[N+](=O)[O-]]

7-morpholino-5-[4-(pyrimidin-2-ylamino)cyclohexoxy]-1,6-naphthyridin-3-ol LFA117 (30 mg, 0.070 mmol), 5-(bromomethyl)-1-methyl-2-nitro-imidazole (17 mg, 0.08 mmol), potassium carbonate (21 mg, 0.15 mmol) and DMF (0.6 mL) was stirred at room temperature under an argon atmosphere for 20 h. The mixture was partitioned between water (20 mL) and EtOAc (2×20 mL) and the combined organic phases were washed with brine (20 mL), dried over MgSO4, filtered and concentrated. The residue was purified via automated flash chromatography using 0-5% MeOH/CH₂Cl as the mobile phase to afford the product. The product was further purified via preparative HPLC using 5-95% 0.005 M NH₄OH/MeCN in 0.005 M NH₄OH/H₂O as the mobile phase to afford the titled compound as an orange solid (14 mg, 34%).

¹H NMR (400 MHz, DMSO-d₆) δ 8.70 (d, J=3.0 Hz, 1H), 8.27 (d, J=4.8 Hz, 2H), 7.85 (dd, J=3.0, 0.8 Hz, 1H), 7.40 (s, 1H), 7.06 (d, J=7.9 Hz, 1H), 6.58-6.51 (m, 2H), 5.45 (s, 2H), 5.44-5.40 (m, 1H), 4.02 (s, 3H), 3.96-3.87 (m, 1H), 3.78-3.71 (m, 4H), 3.48-3.41 (m, 4H), 2.17-2.03 (m, 2H), 1.89-1.70 (m, 6H).

m/z calcd. For C₂₇H₃₁N₉O₅=561.6. Found [M+H]⁺=562.2

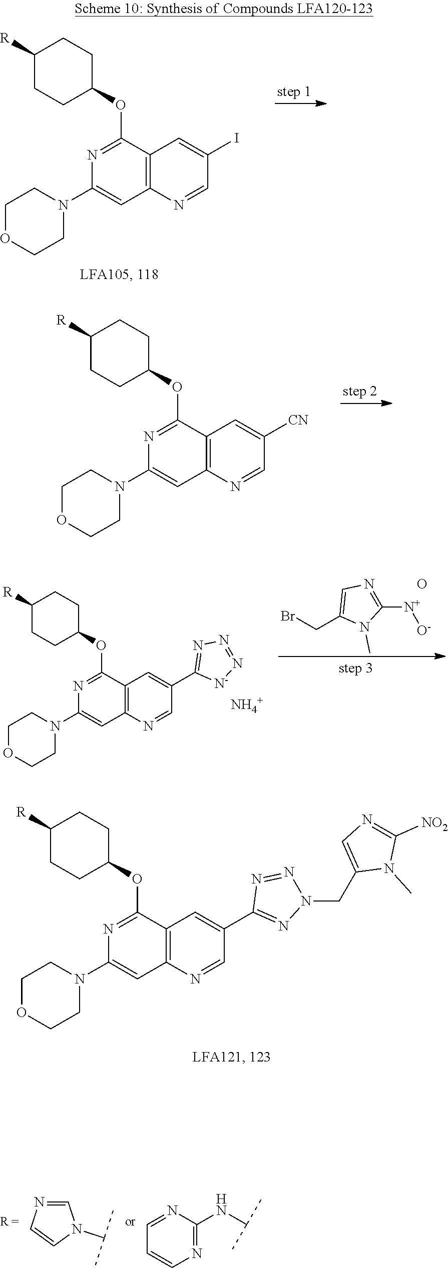

### Step 1

The appropriate aryl iodide, zinc cyanide (2.05 eq) and DMF (0.2 M) were charged to a 2-5 mL microwave vial and the mixture was de-gassed by sparging with argon for 5 minutes. Tetrakis(triphenylphosphine)palladium(0) (0.1 eq)) was added, the vial sealed and the mixture sparged with argon for a further 5 minutes. The mixture was heated to 120° C. and stirred for 20-24 h. After cooling to RT, water (approx. same volume as DMF in the reaction) was added resulting in a precipitate forming. The precipitate was filtered and the collected solid was washed with water (3×) and dried by drawing air through the filter cake for 1 h. The solid was dissolved/suspended in MeOH and concentrated to give the crude product as a yellow solid. The crude product was purified via automated flash chromatography using 2 M NH₃/MeOH in CH₂Cl₂ as the mobile phase to afford the aryl nitrile compounds.

### Step 2

The appropriate aryl nitrile, copper(I) iodide (0.2 eq) and sodium azide (1.1 eq) were charged to a microwave vial. The vial was sealed and purged with a stream of argon. DMF (0.12 M) was added and the mixture was heated to 120° C. and stirred for 20-24 h. After cooling to RT, 1 M HCl (aq.) (approx. 2×volume of DMF in the reaction) was added resulting in precipitate formation. The mixture was allowed to stand at RT for 1 h before the precipitate was filtered and the collected solid washed with water (3×) and dried to give the crude product HCl salt as a red solid. The crude product HCl salt was suspended in MeOH (2 mL) and was loaded onto an SCX column. The column was flushed with MeOH followed by 2 M NH₃/MeOH. The NH₃/MeOH fraction was concentrated to afford the aryl tetrazole compounds as the ammonia salts.

### Step 3

An RB flask was charged with the appropriate aryl tetrazole ammonia salt, alkyl halide LFA007 (1.5-2.5 eq), potassium carbonate (2.5 eq) and DMF (0.06 M). The mixture was stirred at room temperature under an argon atmosphere for 18-24 h. The mixture was concentrated and the crude product was purified via automated flash chromatography using 2 M NH₃/MeOH in CH₂Cl₂ as the mobile phase and/or via automated reverse phase preparative HPLC using 5-95% 0.005 M NH₄OH/MeCN in 0.005 M NH₄OH/H₂O or 5-95% 0.1% HCOOH/MeCN in 0.1% HCOOH/H₂O as the mobile phase to afford the alkylated tetrazole compounds.

### 7-morpholino-5-[4-(pyrimidin-2-ylamino)cyclohexoxy]-1,6-naphthyridine-3-carbonitrile (LFA119)

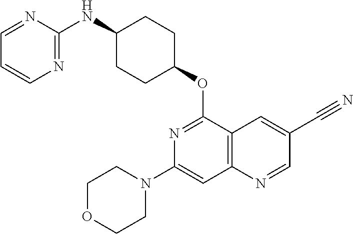 [SMILES: N#Cc1cnc2cc(N3CCOCC3)nc(O[C@H]3CC[C@@H](Nc4ncccn4)CC3)c2c1]

Prepared according to step 1 of Scheme 10 starting with LFA105 to afford the titled compound in 84% yield.

¹H NMR (400 MHz, CDCl₃) δ 8.84 (d, J=2.2 Hz, 1H), 8.56 (dd, J=2.2, 0.8 Hz, 1H), 8.29 (d, J=4.8 Hz, 2H), 6.55 (t, J=4.8 Hz, 1H), 6.50 (d, J=0.8 Hz, 1H), 5.42 (dt, J=4.7, 2.1 Hz, 1H), 5.33 (d, J=7.9 Hz, 1H), 4.08-3.95 (m, 1H), 3.87-3.79 (m, 4H), 3.70-3.60 (m, 4H), 2.25-2.13 (m, 2H), 2.08-1.97 (m, 2H), 1.94-1.84 (m, 2H), 1.83-1.69 (m, 2H) (overlapping with H₂O peak).

m/z calcd. C₂₃H₂₅N₇O₂=431.5. Found [M+H]⁺=432.2.

### Ammonia; N-[4-[[7-morpholino-3-(2H-tetrazol-5-yl)-1,6-naphthyridin-5-yl]oxy]cyclohexyl]pyrimidin-2-amine (LFA120)

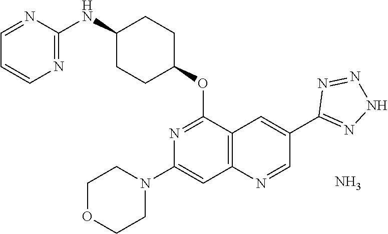

Prepared according to step 2 of Scheme 10 starting with LFA119 to afford the titled compound in 64% yield.

¹H NMR (400 MHz, Methanol-d₄) δ 9.40 (s, 1H), 9.10 (d, J=2.0 Hz, 1H), 8.25 (d, J=4.8 Hz, 2H), 6.56 (t, J=4.8 Hz, 1H), 6.53 (s, 1H), 5.56-5.49 (m, 1H), 4.01-3.89 (m, 1H), 3.88-3.78 (m, 4H), 3.70-3.54 (m, 4H), 2.34-2.21 (m, 2H), 2.05-1.78 (m, 6H). NH signals not directly observed.

m/z calcd. C₂₃H₂₆N₁₀O₂=474.2. Found [M+H]⁺=475.2 (for parent ion).

### N-[4-[[3-[2-[(3-Methyl-2-nitro-imidazol-4-yl)methyl]tetrazol-5-yl]-7-morpholino-1,6-naphthyridin-5-yl]oxy]cyclohexyl]pyrimidin-2-amine (LFA121)

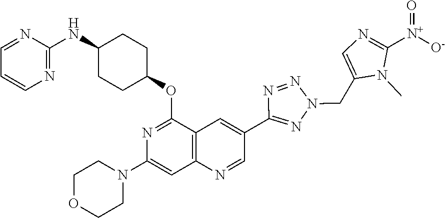 [SMILES: Cn1c(Cn2nnc(-c3cnc4cc(N5CCOCC5)nc(O[C@H]5CC[C@@H](Nc6ncccn6)CC5)c4c3)n2)cnc1[N+](=O)[O-]]

Prepared according to step 3 of Scheme 10 starting with LFA120 to afford the titled compound in 8% yield.

¹H NMR (400 MHz, DMSO-d₆) δ 9.34 (d, J=2.3 Hz, 1H), 8.86 (d, J=2.5 Hz, 1H), 8.26 (d, J=4.7 Hz, 2H), 7.43 (s, 1H), 7.17 (d, J=7.6 Hz, 1H), 6.62 (s, 1H), 6.53 (t, J=4.7 Hz, 1H), 6.33 (s, 2H), 5.42-5.36 (m, 1H), 4.02 (s, 3H), 3.94-3.81 (m, 1H), 3.79-3.70 (m, 4H), 3.61-3.54 (m, 4H), 2.23-2.13 (m, 2H), 1.92-1.68 (m, 6H).

m/z calcd. C₃₁H₁₃N₁₁O₄=613.6. Found [M+H]⁺=614.2.

### 5-(4-Imidazol-1-ylcyclohexoxy)-7-morpholino-1,6-naphthyridine-3-carbonitrile (LFA122-1)

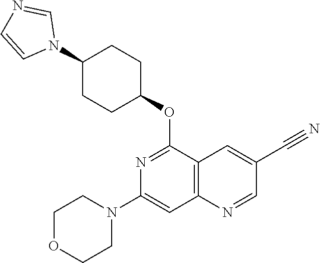 [SMILES: N#Cc1cnc2cc(N3CCOCC3)nc(O[C@H]3CC[C@@H](n4ccnc4)CC3)c2c1]

Prepared according to step 1 of Scheme 10 starting with LFA118 to afford the titled compound in 95% yield.

¹H NMR (400 MHz, CDCl₃) δ 8.85 (d, J=2.2 Hz, 1H), 8.56 (dd, J=2.2, 0.8 Hz, 1H), 7.90 (br. s, 1H), 7.17 (br. s, 1H), 7.10 (br. s, 1H), 6.53 (d, J=0.9 Hz, 1H), 5.55-5.48 (m, 1H), 4.24-4.11 (m, 1H), 3.88-3.77 (m, 4H), 3.68-3.60 (m, 4H), 2.47-2.34 (m, 2H), 2.26-2.06 (m, 4H), 1.99-1.80 (m, 2H). Water peak overlapping with signal at 1.99-1.80 ppm. Imidazole protons appear as broad singlets.

m/z calcd. C₂₂H₂₄N₆O₂=404.2. Found [M+H]⁺=405.2.

### Ammonia; 4-[5-(4-imidazol-1-ylcyclohexoxy)-3-(1,2,3-triaza-4-azanidacyclopenta-2,5-dien-5-yl)-1,6-naphthyridin-7-yl]morpholine (LFA122-2)

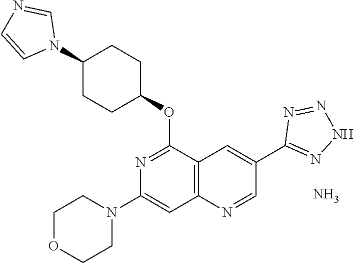

Prepared according to step 2 of Scheme 10 starting with LFA122-1. No precipitation was observed on the addition of 1 M HCl (aq.). Therefore, the mixture was concentrated to remove most of the water and DMF and purified by SCX as in step 2 to afford the titled compound in 52% yield.

¹H NMR (400 MHz, Methanol-d₄) δ 9.41 (d, J=2.2 Hz, 1H), 9.10 (dd, J=2.2, 0.8 Hz, 1H), 8.03 (d, J=1.3 Hz, 1H), 7.45 (d, J=1.4 Hz, 1H), 7.05 (t, J=1.2 Hz, 1H), 6.51 (d, J=0.9 Hz, 1H), 5.59-5.52 (m, 1H), 4.38-4.22 (m, 1H), 3.90-3.72 (m, 4H), 3.63-3.52 (m, 4H), 2.52-2.26 (m, 4H), 2.14-1.99 (m, 2H), 1.99-1.81 (m, 2H). NH₄ not directly observed.

m/z calcd. C₂₂H₂₅N₉O₂=447.2. Found [M+H]⁺=448.2 (for parent ion).

### 4-[5-(4-imidazol-1-ylcyclohexoxy)-3-[2-[(3-methyl-2-nitro-imidazol-4-yl)methyl]tetrazol-5-yl]-1,6-naphthyridin-7-yl]morpholine (LFA123)

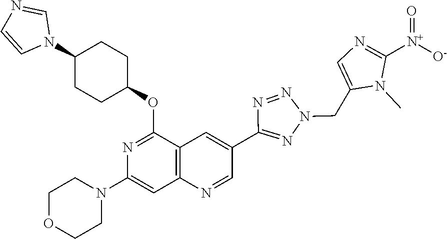 [SMILES: Cn1c(Cn2nnc(-c3cnc4cc(N5CCOCC5)nc(O[C@H]5CC[C@@H](n6ccnc6)CC5)c4c3)n2)cnc1[N+](=O)[O-]]

Prepared according to step 3 of Scheme 10 starting with LFA122 to afford the titled compound in 33% yield.

¹H NMR (400 MHz, Methanol-d₄) δ 9.34 (d, J=2.2 Hz, 1H), 9.07 (dd, J=2.3, 0.8 Hz, 1H), 8.51 (d, J=1.6 Hz, 1H), 7.58 (t, J=1.6 Hz, 1H), 7.43 (s, 1H), 7.34 (t, J=1.5 Hz, 1H), 6.56 (d, J=0.9 Hz, 1H), 6.23 (s, 2H), 5.63-5.58 (m, 1H), 4.45 (tt, J=11.7, 3.8 Hz, 1H), 4.14 (s, 3H), 3.88-3.79 (m, 4H), 3.69-3.59 (m, 4H), 2.47-2.38 (m, 2H), 2.37-2.23 (m, 2H), 2.19-2.10 (m, 2H), 2.05-1.92 (m, 2H).

m/z calcd. C₂₇H₃₀N₁₂O₄=586.3. Found [M+H]⁺=587.2.

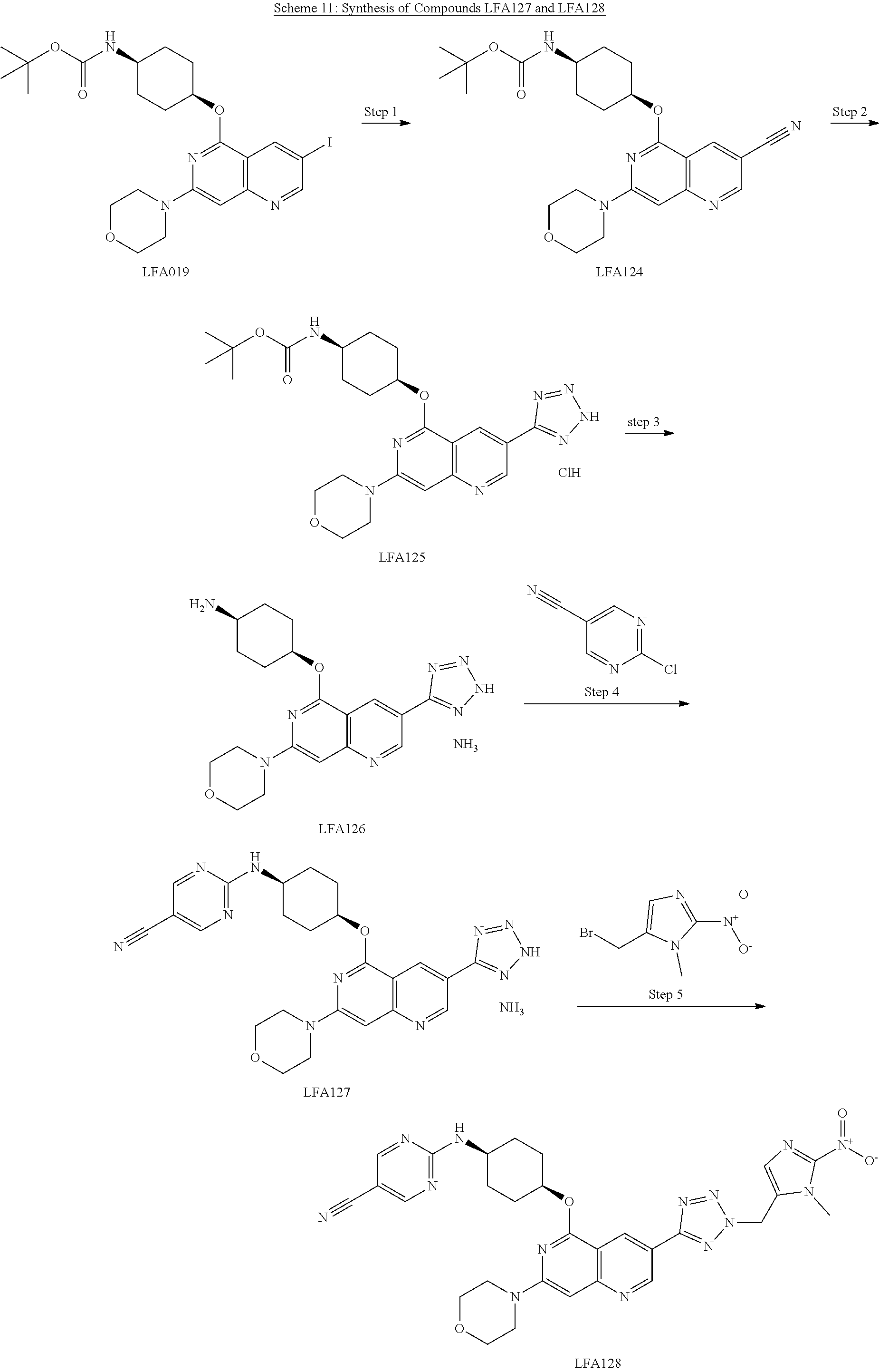

### Step 1

### tert-Butyl N-[4-[(3-cyano-7-morpholino-1,6-naphthyridin-5-yl)oxy]cyclohexyl]carbamate (LFA124)

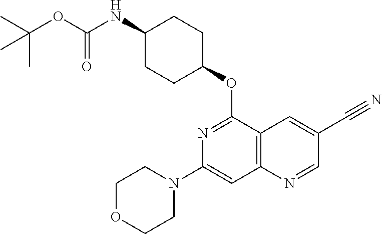 [SMILES: CC(C)(C)OC(=O)N[C@H]1CC[C@@H](Oc2nc(N3CCOCC3)cc3ncc(C#N)cc23)CC1]

A mixture of zinc cyanide (127 mg, 1.08 mmol) and tert-butyl N-[4-[(3-iodo-7-morpholino-1,6-naphthyridin-5-yl)oxy]cyclohexyl]carbamate LFA019 (300 mg, 0.54 mmol) in DMF (2.7 mL) charged to a 2-5 mL microwave vial was de-gassed by sparging with argon for 5 minutes. Tetrakis(triphenylphosphine)palladium(0) (63 mg, 0.05 mmol) was added, the vial sealed and the mixture sparged with argon for a further 5 minutes. The mixture was heated to 120° C. and stirred for 22 h. After cooling to RT, water (4 mL) was added resulting in a precipitate forming. The precipitate was filtered and the collected solid was washed with water (3×4 mL) and dried by drawing air through the filter cake for 1 h. The solid was dissolved/suspended in MeOH (40 mL) and concentrated in to give the crude product as a dark yellow solid (335 mg). The crude product was purified via automated flash chromatography using 0-80% EtOAc/n-heptane as the mobile phase to afford the titled compound as a yellow solid (172 mg, 70%)

¹H NMR (400 MHz, CDCl₃) δ 8.82 (d, J=2.2 Hz, 1H), 8.52 (dd, J=2.2, 0.8 Hz, 1H), 6.50 (d, J=0.9 Hz, 1H), 5.44-5.36 (m, 1H), 4.69-4.49 (m, 1H), 3.90-3.73 (m, 4H), 3.70-3.59 (m, 5H), 2.19-2.07 (m, 2H), 1.97-1.86 (m, 2H), 1.84-1.72 (m, 2H), 1.70-1.55 (m, 2H), 1.46 (s, 9H).

m/z calcd. C₂₄H₃₁N₅O₄=453.5. Found [M+H]⁺=454.2.

### Step 2

### tert-Butyl N-[4-[[7-morpholino-3-(2H-tetrazol-5-yl)-1,6-naphthyridin-5-yl]oxy]cyclohexyl]carbamate; hydrochloride (LFA125)

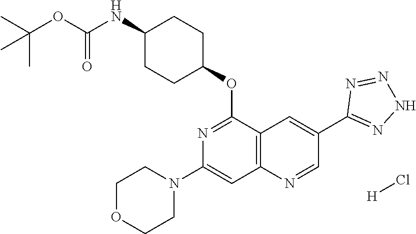 [SMILES: CC(C)(C)OC(=O)N[C@H]1CC[C@@H](Oc2nc(N3CCOCC3)cc3ncc(-c4nn[nH]n4)cc23)CC1 ; Cl]

tert-Butyl N-[4-[(3-cyano-7-morpholino-1,6-naphthyridin-5-yl)oxy]cyclohexyl]carbamate LFA124 (70 mg, 0.15 mmol), copper(I) iodide (6 mg, 0.03 mmol) and sodium azide (11 mg, 0.17 mmol) were charged to a 2-5 mL microwave vial. The vial was sealed, purged with a stream or argon for 5 minutes and maintained under an argon atmosphere. DMF (1.3 mL) was added and the mixture was heated to 120° C. and stirred for 18 h. After cooling to RT, 1 M HCl (aq.) (3 mL) was added resulting in precipitate formation. The mixture was allowed to stand at RT for 1 h before the precipitate was filtered and the collected solid washed with water (3×5 mL) and dried to afford the titled compound as a red solid (67 mg, 81%).

¹H NMR (400 MHz, DMSO-d₆) δ 9.36-9.28 (m, 1H), 8.87 (d, J=2.2 Hz, 1H), 6.84 (d, J=7.6 Hz, 1H), 6.62 (s, 1H), 5.41-5.29 (m, 1H), 3.77-3.69 (m, 4H), 3.64-3.54 (m, 4H), 2.17-2.03 (m, 2H), 1.80-1.62 (m, 6H), 1.38 (s, 9H). BocNH—CH and naphthyridine-NHCl obscured by water peak at 3.34 ppm.

m/z calcd. C₂₄H₃₂N₈O₄=496.3. Found [M+H]⁺=497.2 (for parent ion).

### Step 3

### Ammonia; 4-[[7-morpholino-3-(2H-tetrazol-5-yl)-1,6-naphthyridin-5-yl]oxy]cyclohexanamine (LFA126)

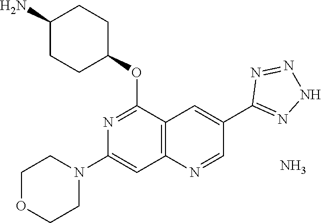

Hydrogen chloride (3.7-4.3 M in 1,4-dioxane) (0.32 mL, 1.2 mmol) was added to a stirred suspension of tert-butyl N-[4-[[7-morpholino-3-(2H-tetrazol-5-yl)-1,6-naphthyridin-5-yl]oxy]cyclohexyl]carbamate hydrochloride LFA125 (64 mg, 0.12 mmol) in MeOH (2.4 mL). The mixture was stirred at room temperature for 18 h. The mixture was concentrated, re-dissolved in MeOH (2 mL) and loaded onto an SCX column (1 g). The column was flushed with MeOH (100 mL) followed by 2 M NH₃/MeOH (60 mL). The NH₃/MeOH fraction was concentrated in vacuo to afford the titled compound as a yellow/orange solid (50 mg, 96%).

¹H NMR (400 MHz, DMSO-d₆) δ 9.41 (d, J=2.2 Hz, 1H), 8.78 (dd, J=2.2, 0.8 Hz, 1H), 7.90 (s, 2H), 6.58 (d, J=0.9 Hz, 1H), 5.49-5.39 (m, 1H), 3.79-3.68 (m, 4H), 3.55-3.47 (m, 4H), 3.22-3.13 (m, 1H), 2.28-2.14 (m, 2H), 1.99-1.66 (m, 6H). Tetrazole-NH·NH₃ not directly observed.

m/z calcd. C₁₉H₂₄N₈O₂=396.2. Found [M+H]⁺=397.2 (for parent ion).

### Step 4

### Ammonia; 2-[[4-[[7-morpholino-3-(2H-tetrazol-5-yl)-1,6-naphthyridin-5-yl]oxy]cyclohexyl]amino]pyrimidine-5-carbonitrile (LFA127)

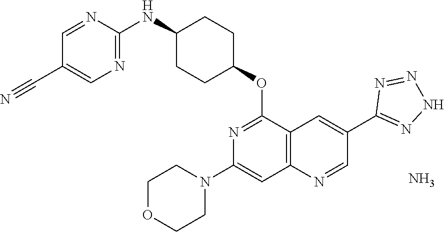

Triethylamine (0.05 mL, 0.36 mmol) was added to a stirred suspension of ammonia; 4-[[7-morpholino-3-(2H-tetrazol-5-yl)-1,6-naphthyridin-5-yl]oxy]cyclohexanamine LFA126 (49 mg, 0.12 mmol) and 2-chloropyrimidine-5-carbonitrile (18 mg, 0.13 mmol) in EtOH (1.1 mL). The mixture was stirred at room temperature for 6.5 h. The reaction mixture was concentrated and the residue was purified via automated flash chromatography using 0-20% 2 M NH₃/MeOH in CH₂Cl₂ as the mobile phase to afford the titled compound as a bright red/orange solid (50 mg, 82%).

¹H NMR (400 MHz, DMSO-d₆) δ 9.34 (d, J=2.3 Hz, 1H), 8.89 (dd, J=2.4, 0.8 Hz, 11H), 8.72 (d, J=3.0 Hz, 1H), 8.64 (d, J=3.0 Hz, 1H), 8.40 (d, J=7.8 Hz, 1H), 6.63 (d, J=0.9 Hz, 1H), 5.46-5.37 (m, 1H), 4.05-3.91 (m, 1H), 3.79-3.70 (m, 4H), 3.63-3.54 (m, 4H), 2.26-2.09 (m, 2H), 1.93-1.71 (m, 6H). (tetrazole NH/NH₃ salt not directly observed).

m/z calcd. C₂₄H₂₅N1₁O₂=499.2. Found [M+H]⁺=500.2 (for parent ion).

### Step 5

### 2-[[4-[[3-[2-[(3-Methyl-2-nitro-imidazol-4-yl)methyl]tetrazol-5-yl]-7-morpholino-1,6-naphthyridin-5-yl]oxy]cyclohexyl]amino]pyrimidine-5-carbonitrile (LFA128)

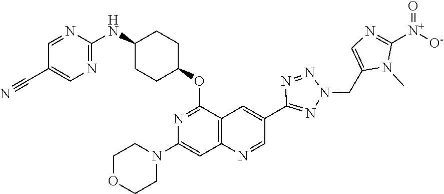 [SMILES: Cn1c(Cn2nnc(-c3cnc4cc(N5CCOCC5)nc(O[C@H]5CC[C@@H](Nc6ncc(C#N)cn6)CC5)c4c3)n2)cnc1[N+](=O)[O-]]

5-(bromomethyl)-1-methyl-2-nitro-imidazole (23 mg, 0.10 mmol) and potassium carbonate (24 mg, 0.17 mmol) were added to a stirred solution of ammonia; 2-[[4-[[7-morpholino-3-(2H-tetrazol-5-yl)-1,6-naphthyridin-5-yl]oxy]cyclohexyl]amino]pyrimidine-5-carbonitrile LFA127 (36 mg, 0.07 mmol) in DMF (1.4 mL). The mixture was stirred at room temperature for 18 h. The reaction mixture was filtered and the filtrate concentrated. crude product as a dark brown gum. The residue was purified via automated flash chromatography using 0-8% 2 M NH₃/MeOH in CH₂Cl₂ as the mobile phase and then by preparative HPLC using 5-95% 0.005 M NH₄OH/MeCN in 0.005 M NH₄OH/H₂O as the mobile phase to afford the titled compound as a yellow solid (14 mg, 31%).

¹H NMR (400 MHz, DMSO-d₆) δ 9.34 (d, J=2.3 Hz, 1H), 8.85 (dd, J=2.3, 0.8 Hz, 1H), 8.72 (d, J=3.0 Hz, 1H), 8.64 (d, J=3.0 Hz, 1H), 8.43 (d, J=7.8 Hz, 1H), 7.43 (s, 1H), 6.62 (d, J=0.9 Hz, 1H), 6.33 (s, 2H), 5.45-5.32 (m, 1H), 4.02 (s, 3H), 4.00-3.93 (m, 1H), 3.78-3.70 (m, 4H), 3.63-3.54 (m, 4H), 2.27-2.10 (m, 2H), 1.92-1.74 (m, 6H).

m/z calcd. C₂₉H₃₀N₁₄O₄=638.6. Found [M+H]⁺=639.2.

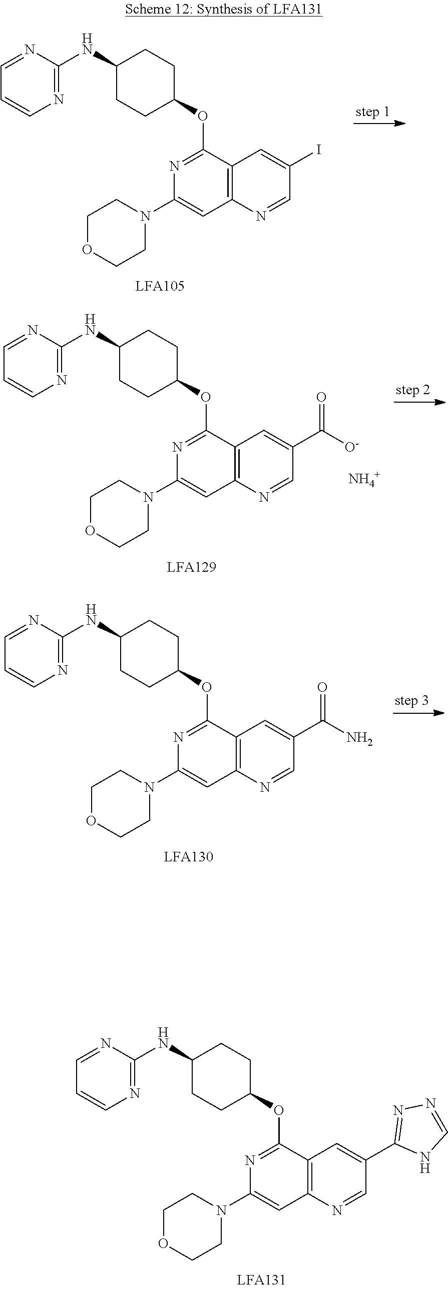 [SMILES: Ic1cnc2cc(N3CCOCC3)nc(O[C@H]3CC[C@@H](Nc4ncccn4)CC3)c2c1 ; O=C([O-])c1cnc2cc(N3CCOCC3)nc(O[C@H]3CC[C@@H](Nc4ncccn4)CC3)c2c1 ; [NH4+] ; NC(=O)c1cnc2cc(N3CCOCC3)nc(O[C@H]3CC[C@@H](Nc4ncccn4)CC3)c2c1 ; c1cnc(N[C@H]2CC[C@@H](Oc3nc(N4CCOCC4)cc4ncc(-c5nnc[nH]5)cc34)CC2)nc1]

### Step 1

### Ammonium; 7-morpholino-5-[4-(pyrimidin-2-ylamino)cyclohexoxy]-1,6-naphthyridine-3-carboxyl ate (LFA129)

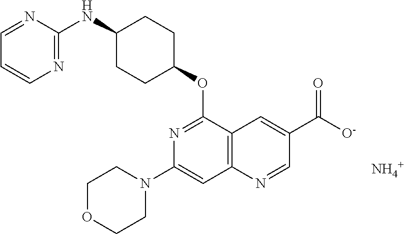 [SMILES: O=C([O-])c1cnc2cc(N3CCOCC3)nc(O[C@H]3CC[C@@H](Nc4ncccn4)CC3)c2c1 ; [NH4+]]

N-[4-[(3-Iodo-7-morpholino-1,6-naphthyridin-5-yl)oxy]cyclohexyl]pyrimidin-2-amine LFA105 (200 mg, 0.38 mmol), [methyl(diphenyl)silyl]formic acid (137 mg, 0.56 mmol), potassium trimethylsilanolate (107 mg, 0.75 mmol), bis(dibenzylideneacetone)palladium(0) (11 mg, 0.02 mmol) and 9,9-dimethyl-4,5-bis(diphenylphosphino)xanthene (11 mg, 0.02 mmol) were charged to a reactivial. Toluene (2.25 mL) was added, the vial sealed and the reaction 40° C. and stirred for 30 minutes. The mixture was concentrated and the residue was dissolved in MeOH and loaded onto an SCX column (5 g). The column was washed with MeOH and eluted with 2 M NH₃/MeOH. The NH₃/MeOH fraction was concentrated to afford the titled compound as a yellow solid (176 mg, 100%).

¹H NMR (400 MHz, DMSO-d₆) δ9.18 (d, J=2.1 Hz, 1H), 8.73 (dd, J=2.2, 0.8 Hz, 1H), 8.25 (d, J=4.7 Hz, 2H), 7.24 (d, J=7.8 Hz, 1H), 6.56 (d, J=0.9 Hz, 1H), 6.52 (t, J=4.7 Hz, 1H), 5.42-5.32 (m, 1H), 3.94-3.82 (m, 1H), 3.74 (dd, J=5.8, 4.0 Hz, 4H), 3.59-3.51 (m, 4H), 2.21-2.07 (m, 2H), 1.89-1.71 (m, 6H).

m/z calcd. C₂₃H₂₆N₆O₄=450.2. Found [M+H]⁺=451.2.

### Step 2

### 7-morpholino-5-[4-(pyrimidin-2-ylamino)cyclohexoxy]-1,6-naphthyridine-3-carboxamide (LFA130)

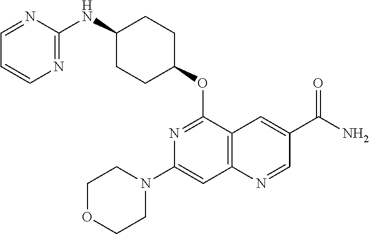 [SMILES: NC(=O)c1cnc2cc(N3CCOCC3)nc(O[C@H]3CC[C@@H](Nc4ncccn4)CC3)c2c1]

Ammonium; 7-morpholino-5-[4-(pyrimidin-2-ylamino)cyclohexoxy]-1,6-naphthyridine-3-carboxylate LFA129 (76 mg, 0.16 mmol), 0-(7-aza-1H-benzotriazol-1-yl)-N,N,N′,N′-tetramethyluronium hexafluorophosphate (124 mg, 0.33 mmol) and ammonium chloride (87 mg, 1.63 mmol) were suspended in DMF (1 mL). DIPEA (0.14 mL, 0.81 mmol) was added and the reaction allowed to stir at room temperature for 19 h. The reaction was diluted with CH₂Cl₂ was washed with water (4×). The combined organics were washed with brine, dried (Na₂SO₄) and concentrated. The residue was purified via automated flash chromatography using 0-7% MeOH/CH₂Cl₂ as the mobile phase to afford the titled compound as a yellow solid (31 mg, 42%).

¹H NMR (400 MHz, CDCl₃) δ 9.21 (d, J=2.4 Hz, 1H), 8.74 (dd, J=2.4, 0.8 Hz, 1H), 8.28 (d, J=4.8 Hz, 2H), 6.57-6.49 (m, 2H), 5.46-5.37 (m, 1H), 5.33 (d, J=8.0 Hz, 1H), 4.07-3.95 (m, 1H), 3.84 (dd, J=5.7, 4.1 Hz, 4H), 3.64-3.57 (m, 4H), 2.20-2.12 (m, 2H), 1.99-1.68 (m, 8H).

m/z calcd. C₂₃H₂₇N₇O₃=449.5. Found [M+H]⁺=450.2.

### Step 3

### N-[4-[[7-Morpholino-3-(4H-1,2,4-triazol-3-yl)-1,6-naphthyridin-5-yl]oxy]cyclohexyl]pyrimidin-2-amine (LFA131)

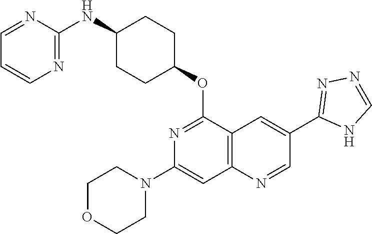 [SMILES: c1cnc(N[C@H]2CC[C@@H](Oc3nc(N4CCOCC4)cc4ncc(-c5nnc[nH]5)cc34)CC2)nc1]

7-Morpholino-5-[4-(pyrimidin-2-ylamino)cyclohexoxy]-1,6-naphthyridine-3-carboxamide LF130 (31 mg, 0.07 mmol) was suspended in N,N-dimethylformamide dimethyl acetal (0.52 mL, 3.89 mmol) and heated to 90° C. for 1 hour. The mixture was concentrated and the resulting residue was suspended in acetic acid (1 mL) and hydrazine monohydrate (0.21 mL, 4.25 mmol) was added. The mixture was heated to 90° C. and stirred for 2 h. The reaction was concentrated and the residue was partitioned between CH₂Cl₂ and sat. NaHCO₃ (aq.) The aqueous phase was extracted with 5% MeOH/CH₂Cl₂. The organic phase was dried (Na₂SO₄), concentrated and the residue was purified via automated flash chromatography using 0-7% MeOH/CH₂Cl₂ as the mobile phase, then by preparative HPLC using 5-95% 0.005 M NH₄OH/MeCN in 0.005 M NH₄OH/H₂O as the mobile phase to afford the titled compound as a pale yellow solid (9 mg, 28%).

¹H NMR (400 MHz, DMSO-d₆) δ 9.38 (d, J=2.2 Hz, 1H), 8.84 (dd, J=2.3, 0.8 Hz, 1H), 8.55 (s, 1H), 8.26 (d, J=4.8 Hz, 2H), 7.20 (d, J=7.7 Hz, 1H), 6.59 (d, J=0.9 Hz, 1H), 6.53 (t, J=4.8 Hz, 1H), 5.45-5.32 (m, 1H), 3.93-3.83 (m, 1H), 3.75 (dd, J=5.8, 3.9 Hz, 4H), 3.59-3.50 (m, 4H), 2.24-2.10 (m, 2H), 1.89-1.72 (m, 6H). Triazole NH not observed in NMR.

m/z calcd. C₂₄H₂₇N₉O₂=473.5. Found [M+H]⁺=474.2.

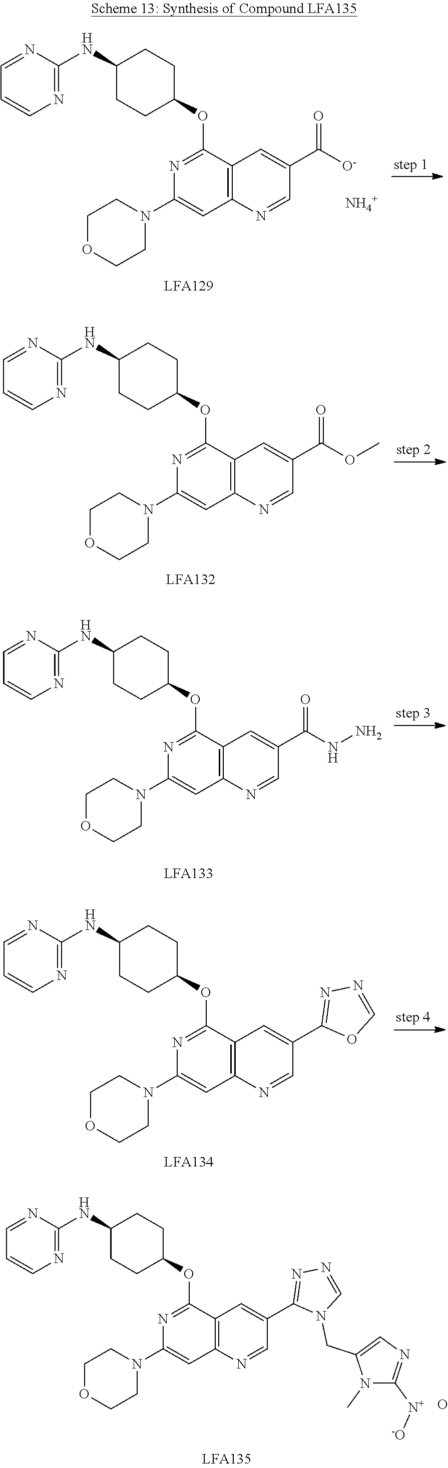

### Methyl 7-morpholino-5-[4-(pyrimidin-2-ylamino)cyclohexoxy]-1,6-naphthyridine-3-carboxylate (LFA132)

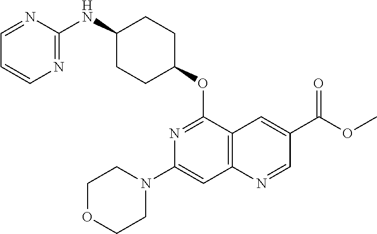 [SMILES: COC(=O)c1cnc2cc(N3CCOCC3)nc(O[C@H]3CC[C@@H](Nc4ncccn4)CC3)c2c1]

Ammonium; 7-morpholino-5-[4-(pyrimidin-2-ylamino)cyclohexoxy]-1,6-naphthyridine-3-carboxylate LFA129 (400 mg, 0.86 mmol) was suspended in MeOH (8 mL) in a microwave vial, sulfuric acid (14 mL, 2.57 mmol) was added, the vial capped and the solution microwaved at 100° C. for 8 h and then basified by addition of sat. NaHCO₃ (aq.) and concentrated. The residue was partitioned between water (50 mL) and CH₂Cl₂ (3×50 mL) and the combined organic phases were dried via a hydrophobic frit and concentrated. The residue was purified via automated flash chromatography using 0-10% MeOH/CH₂Cl₂ as the mobile phase to afford the titled compound a yellow solid (345 mg, 86%)

¹H NMR (400 MHz, CDCl₃) δ 9.32 (d, J=2.2 Hz, 1H), 8.91 (dd, J=2.2, 0.8 Hz, 1H), 8.31 (d, J=4.8 Hz, 2H), 6.57 (d, J=0.9 Hz, 1H), 6.55 (t, J=4.8 Hz, 1H), 5.49-5.43 (m, 1H), 5.18 (d, J=8.0 Hz, 1H), 4.09-3.95 (m, 1H), 4.01 (s, 3H), 3.91-3.84 (m, 4H), 3.68-3.61 (m, 4H), 2.22 (dd, J=10.9, 5.6 Hz, 2H), 2.07-1.98 (m, 2H), 1.97-1.75 (m, 4H).

m/z calcd. C₂₄H₂₈N₆O₄=464.2. Found [M+H]⁺=465.0.

### 7-Morpholino-5-[4-(pyrimidin-2-ylamino)cyclohexoxy]-1,6-naphthyridine-3-carbohydrazide (LFA133)

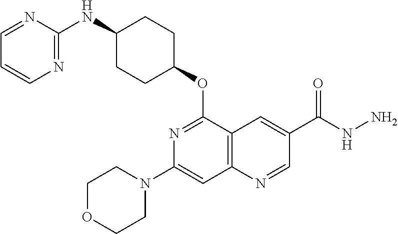 [SMILES: NNC(=O)c1cnc2cc(N3CCOCC3)nc(O[C@H]3CC[C@@H](Nc4ncccn4)CC3)c2c1]

Methyl 7-morpholino-5-[4-(pyrimidin-2-ylamino)cyclohexoxy]-1,6-naphthyridine-3-carboxylate LFA132 (290 mg, 0.62 mmol) was suspended in EtOH (4.9 mL) in a microwave vial, hydrazine monohydrate (0.30 mL, 6.24 mmol) was added and the vial was capped. The mixture was heated at 100° C. for 16 h and then at 120° C. for 5 h. The resulting suspension was cooled to room temperature and yellow solid filtered off, washed with MeOH (10 mL) and dried under suction. The filtrate concentrated to give an orange solid. The filtrate and solid were combined, and the residue was purified via automated flash chromatography using 0-10% MeOH/CH₂Cl₂ as the mobile phase to afford the titled compound as a yellow solid (224 mg, 77%)

¹H NMR (400 MHz, DMSO-d₆) δ 9.99 (s, 1H), 9.12 (d, J=2.3 Hz, 1H), 8.70 (dd, J=2.3, 0.8 Hz, 1H), 8.26 (d, J=4.8 Hz, 2H), 7.08 (d, J=7.8 Hz, 1H), 6.56 (d, J=0.8 Hz, 1H), 6.53 (t, J=4.8 Hz, 1H), 5.42-5.33 (m, 1H), 4.62-4.47 (m, 2H), 3.97-3.82 (m, 1H), 3.74 (dd, J=5.7, 4.0 Hz, 4H), 3.61-3.50 (m, 4H), 2.22-2.05 (m, 2H), 1.90-1.69 (m, 6H). m/z calcd. C₂₃H₂₈N₈O₃=464.2. Found [M+H]⁺=465.0

### N-[4-[[7-Morpholino-3-(1,3,4-oxadiazol-2-yl)-1,6-naphthyridin-5-yl]oxy]cyclohexyl]pyrimidin-2-amine (LFA134)

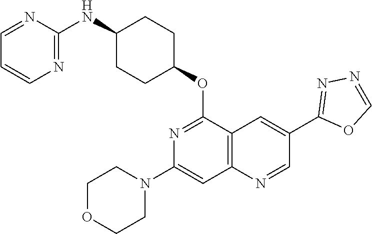 [SMILES: c1cnc(N[C@H]2CC[C@@H](Oc3nc(N4CCOCC4)cc4ncc(-c5nnco5)cc34)CC2)nc1]

7-Morpholino-5-[4-(pyrimidin-2-ylamino)cyclohexoxy]-1,6-naphthyridine-3-carbohydrazide LFA133 (50 mg, 0.11 mmol) was suspended in triethyl orthoformate (1 mL, 6.01 mmol) in a microwave vial and p-toluenesulfonic acid monohydrate (2 mg, 0.01 mmol) was added. The vial was capped and the mixture heated at 100° C. for 16 h. Acetic acid (0.5 mL) was added to the suspension and the mixture heated at 100° C. for a further 1 h. The reaction was cooled to room temperature, diluted with MeOH and loaded onto an SCX cartridge. The cartridge was washed with MeOH and the product eluted with 2 M NH₃ in MeOH/CH₂Cl₂ (1:1) and concentrated to afford the titled compound as an orange solid (54 mg, 105%)

¹H NMR (400 MHz, DMSO-d₆) δ 9.38 (s, 1H), 9.28 (d, J=2.3 Hz, 1H), 8.76 (dd, J=2.3, 0.8 Hz, 1H), 8.24 (d, J=4.8 Hz, 2H), 7.17 (d, J=7.9 Hz, 1H), 6.62 (d, J=0.8 Hz, 1H), 6.51 (t, J=4.8 Hz, 1H), 5.45-5.35 (m, 1H), 3.98-3.79 (m, 1H), 3.75-3.67 (m, 4H), 3.63-3.54 (m, 4H), 2.26-2.04 (m, 2H), 1.90-1.65 (m, 6H).

m/z calcd. C₂₄H₂₆N₈O₃=474.2. Found [M+H]⁺=475.0

### N-[4-[[3-[4-[(3-Methyl-2-nitro-imidazol-4-yl)methyl]-1,2,4-triazol-3-yl]-7-morpholino-1,6-naphthyridin-5-yl]oxy]cyclohexyl]pyrimidin-2-amine (LFA135)

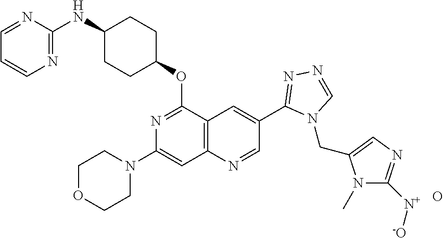

N-[4-[[7-Morpholino-3-(1,3,4-oxadiazol-2-yl)-1,6-naphthyridin-5-yl]oxy]cyclohexyl]pyrimidin-2-amine LFA134 (26 mg, 0.05 mmol) was transferred to a microwave vial, suspended in pyridinium acetate (0.2 mL) and (3-methyl-2-nitro-imidazol-4-yl)methanamine (10 mg, 0.06 mmol) was added. The suspension was heated at 110° C. for 2 h. The mixture was cooled to room temperature, dissolved in CH₂Cl₂ (10 mL) and washed with sat. NaHCO₃ (aq.) (10 mL). The aqueous phase was extracted with CH₂Cl₂ (2×5 mL) and the combined organic phases dried via a hydrophobic frit and concentrated. The residue was purified by preparative HPLC using 5-95% 0.005 M NH₄OH/MeCN in 0.005 M NH₄OH/H₂O as the mobile phase to afford the titled compound as a pale orange solid (2 mg, 7%)

¹H NMR (400 MHz, DMSO-d₆) δ 9.04 (d, J=2.3 Hz, 1H), 8.67 (s, 1H), 8.55 (dd, J=2.3, 0.8 Hz, 1H), 8.25 (d, J=4.7 Hz, 2H), 7.00 (d, J=8.0 Hz, 1H), 6.95 (s, 1H), 6.61 (d, J=0.8 Hz, 1H), 6.53 (t, J=4.7 Hz, 1H), 5.64 (s, 2H), 5.48-5.38 (m, 1H), 3.96-3.83 (m, 1H), 3.80 (s, 3H), 3.77-3.71 (m, 4H), 3.61-3.54 (m, 4H), 2.16-2.05 (m, 2H), 1.87-1.64 (m, 6H).

m/z calcd. C₂₉H₃₂N₁₂O₄=612.3. Found [M+H]⁺=613.0

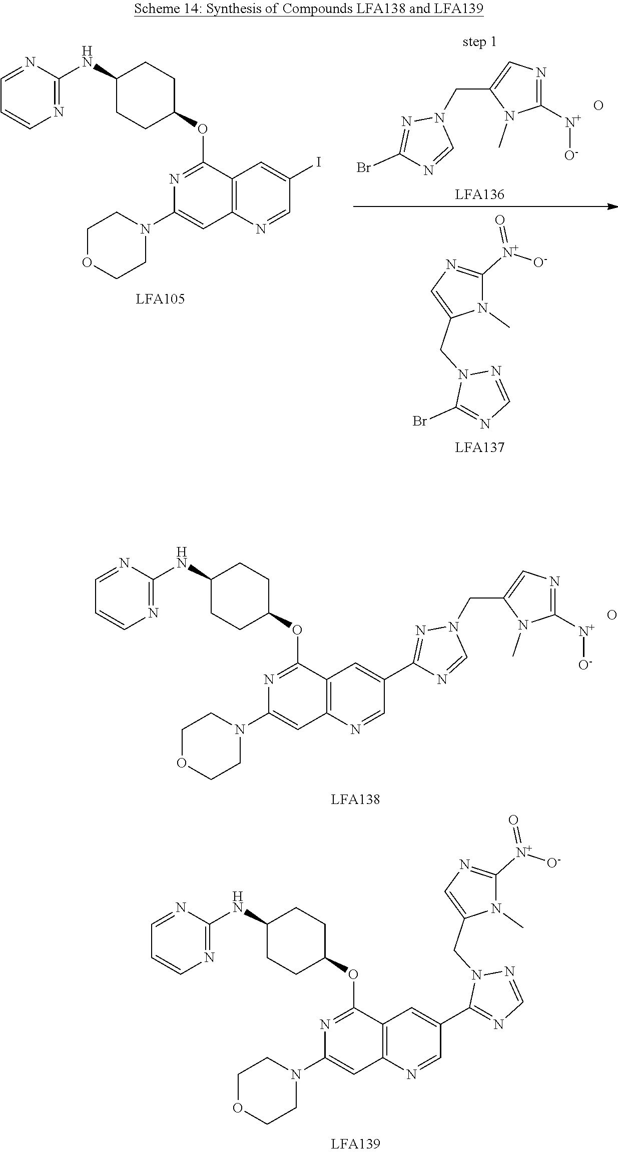

### 3-Bromo-1-[(3-methyl-2-nitro-imidazol-4-yl)methyl]-1,2,4-triazole (LFA136) and 5-Bromo-1-[(3-methyl-2-nitro-imidazol-4-yl)methyl]-1,2,4-triazole (LFA137)

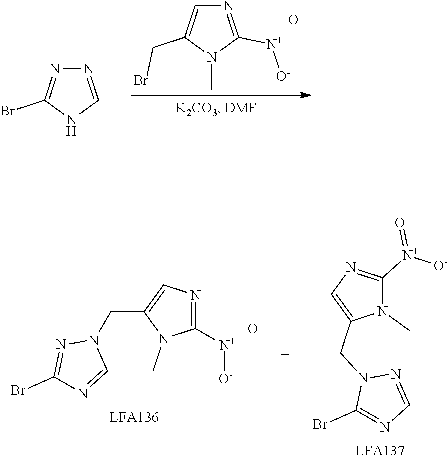

5-(Bromomethyl)-1-methyl-2-nitro-imidazole (286 mg, 1.30 mmol) was added to a stirred solution of 3-bromo-4H-1,2,4-triazole (160 mg, 1.08 mmol) and potassium carbonate (194 mg, 1.41 mmol) in DMF (3.6 mL). The mixture was stirred at room temperature for 5 h. The mixture was diluted with EtOAc (20 mL) and the organic phase washed with water (20 mL) and brine (2×20 mL). The organic phase was dried over Na₂SO₄, filtered and concentrated. The residue was purified via automated flash chromatography using 0-8% MeOH/EtOAc as the mobile phase to afford 3-bromo-1-[(3-methyl-2-nitro-imidazol-4-yl)methyl]-1,2,4-triazole as a pale yellow solid (14 mg, 47%) and 5-bromo-1-[(3-methyl-2-nitro-imidazol-4-yl)methyl]-1,2,4-triazole as a pale yellow solid (42 mg, 14%).

### 3-bromo-1-[(3-methyl-2-nitro-imidazol-4-yl)methyl]-1,2,4-triazole (LFA136)

¹H NMR (400 MHz, DMSO-d₆) δ 8.68 (s, 1H), 7.27 (s, 1H), 5.65 (s, 2H), 3.92 (s, 3H).

Structure (i.e. regioisomer) determined by HSQC and HMBC NMR.

m/z calcd. C₇H7BrN₆O₂=286.0 Found [M+H]⁺=287.1 (for ⁷⁹Br isotope).

5-bromo-1-[(3-methyl-2-nitro-imidazol-4-yl)methyl]-1,2,4-triazole (LFA137)

¹H NMR (400 MHz, DMSO-d₆) δ 8.12 (s, 1H), 7.19 (s, 1H), 5.64 (s, 2H), 3.95 (s, 3H).

Structure (i.e. regioisomer) determined by HSQC and HMBC NMR.

m/z calcd. C₇H7BrN₆O₂=286.0 Found [M+H]⁺=287.1 (for ⁷⁹Br isotope).

### Step 1 (Borylation and Suzuki Coupling)

A mixture of N-[4-[(3-iodo-7-morpholino-1,6-naphthyridin-5-yl)oxy]cyclohexyl]pyrimidin-2-amine LDA105, bis(pinacolato)diboron (1.5 eq), potassium acetate (3.0 eq) and Xphos-Pd-G2 (0.1 eq)) was purged with argon for 5 minutes. DMF (0.1 M) was added and the mixture was sparged with argon for 5 minutes. The mixture was heated to 90° C. and stirred for 2 h. After cooling to RT, the mixture was diluted with EtOAc (approx. 2×volume of DMF) and filtered, rinsing with EtOAc. The organic phase was washed with water and brine, dried over Na₂SO₄ filtered and concentrated to give the crude boronate ester. The boronate ester was re-dissolved in 1,4-dioxane/water (5:1, 0.1 M) and the appropriate triazole bromide LFA136 or LFA137 (1.0 eq), potassium phosphate tribasic (3.0 eq) and Xphos Pd G2 (0.1 eq)) were added. The mixture was sparged with argon for 5 minutes. The mixture was heated to 90° C. and stirred for 1.5-3 h. The mixture was diluted with EtOAc (approx. 20×reaction solvent volume) and filtered. The organic phase was washed with water and brine, dried over Na₂SO₄, filtered and concentrated. The residue was purified via automated flash chromatography using 2 M NH₃/MeOH in CH₂Cl₂ as the mobile phase and/or by preparative HPLC using 5-95% 0.005 M NH₄OH/MeCN in 0.005 M NH₄OH/H₂O as the mobile phase to afford compounds LFA138 and LFA139.

### N-[4-[[3-[1-[(3-Methyl-2-nitro-imidazol-4-yl)methyl]-1,2,4-triazol-3-yl]-7-morpholino-1,6-naphthyridin-5-yl]oxy]cyclohexyl]pyrimidin-2-amine (LFA138)

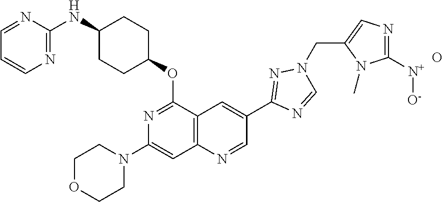

Prepared according to step 1 of Scheme 14 starting with 3-bromo-1-[(3-methyl-2-nitro-imidazol-4-yl)methyl]-1,2,4-triazole LFA136 to afford the titled compound in 45% yield.

¹H NMR (400 MHz, DMSO-d₆) δ 9.33 (d, J=2.2 Hz, 1H), 8.79 (dd, J=2.2, 0.8 Hz, 1H), 8.77 (s, 1H), 8.26 (d, J=4.7 Hz, 2H), 7.29 (s, 1H), 7.18 (d, J=7.6 Hz, 1H), 6.59 (d, J=0.9 Hz, 1H), 6.53 (t, J=4.8 Hz, 1H), 5.74 (s, 2H), 5.42-5.31 (m, 1H), 4.02 (s, 3H), 3.94-3.81 (m, 1H), 3.79-3.69 (m, 4H), 3.63-3.48 (m, 4H), 2.26-2.07 (m, 2H), 1.95-1.63 (m, 6H).

m/z calcd. C₂₉H₃₂N₁₂O₄=612.3. Found [M+H]⁺=613.2

### N-[4-[[3-[2-[(3-Methyl-2-nitro-imidazol-4-yl)methyl]-1,2,4-triazol-3-yl]-7-morpholino-1,6-naphthyridin-5-yl]oxy]cyclohexyl]pyrimidin-2-amine (LFA139)

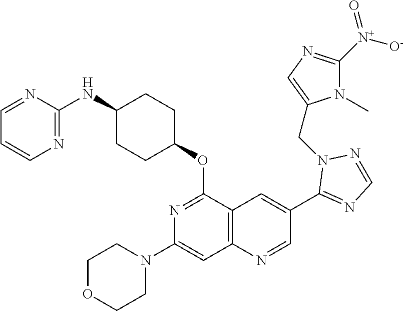 [SMILES: Cn1c(Cn2ncnc2-c2cnc3cc(N4CCOCC4)nc(O[C@H]4CC[C@@H](Nc5ncccn5)CC4)c3c2)cnc1[N+](=O)[O-]]

Prepared according to step 1 of Scheme 14 starting with 5-bromo-1-[(3-methyl-2-nitro-imidazol-4-yl)methyl]-1,2,4-triazole LFA137 to afford the titled compound in 7% yield.

¹H NMR (400 MHz, DMSO-d₆) δ 9.10 (d, J=2.3 Hz, 1H), 8.61 (dd, J=2.4, 0.7 Hz, 1H), 8.24 (d, J=4.7 Hz, 2H), 8.17 (s, 1H), 7.04 (s, 1H), 7.01 (d, J=8.0 Hz, 1H), 6.63 (d, J=0.9 Hz, 1H), 6.53 (t, J=4.8 Hz, 1H), 5.83 (s, 2H), 5.46-5.40 (m, 1H), 3.92-3.84 (m, 1H), 3.88 (s, 3H), 3.78-3.71 (m, 4H), 3.62-3.51 (m, 4H), 2.18-2.04 (m, 2H), 1.89-1.64 (m, 6H).

m/z calcd. C₂₉H₃₂N₁₂O₄=612.3. Found [M+H]⁺=613.2

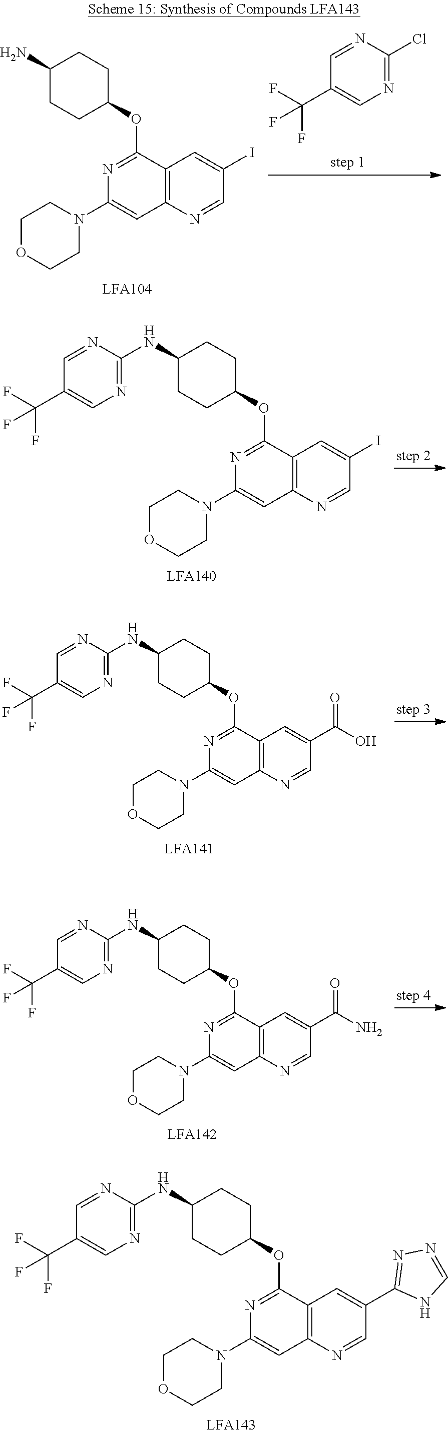

### N-[4-[(3-Iodo-7-morpholino-1,6-naphthyridin-5-yl)oxy]cyclohexyl]-5-(trifluoromethyl)pyrimidin-2-amine (LFA140)

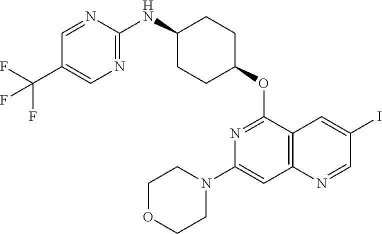 [SMILES: FC(F)(F)c1cnc(N[C@H]2CC[C@@H](Oc3nc(N4CCOCC4)cc4ncc(I)cc34)CC2)nc1]

A mixture of 4-[(3-Iodo-7-morpholino-1,6-naphthyridin-5-yl)oxy]cyclohexanamine LFA104 (250 mg, 0.55 mmol), 2-chloro-5-(trifluoromethyl)pyrimidine (110 mg, 0.61 mmol), triethylamine (0.23 mL, 1.65 mmol) in EtOH (5 mL) was stirred at room temperature for 18 h. The mixture was concentrated and the residue dissolved in CH₂Cl₂, and washed with water. The combined organic phases were dried via passing through a hydrophobic frit, concentrated and the residue was purified via automated flash chromatography using 0-100% EtOAc/n-heptane as the mobile phase to afford the titled compound as a yellow solid (251 mg, 76%).

¹H NMR (400 MHz, DMSO-d₆) δ 8.89 (d, J=2.2 Hz, 1H), 8.62 (s, 1H), 8.59-8.53 (m, 2H), 8.10 (d, J=8.2 Hz, 1H), 6.48 (d, J=0.8 Hz, 1H), 5.45-5.32 (m, 1H), 4.03-3.93 (m, 1H), 3.78-3.68 (m, 4H), 3.57-3.43 (m, 4H), 2.19-2.05 (m, 2H), 1.90-1.66 (m, 6H).

m/z calcd. C₂₃H₂₄F₃N₆O₂=600.4. Found [M+H]⁺=601.0.

### 7-Morpholino-5-[4-[[5-(trifluoromethyl)pyrimidin-2-yl]amino]cyclohexoxy]-1,6-naphthyridine-3-carboxylic acid (LFA141)

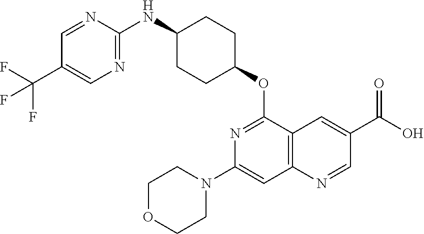 [SMILES: O=C(O)c1cnc2cc(N3CCOCC3)nc(O[C@H]3CC[C@@H](Nc4ncc(C(F)(F)F)cn4)CC3)c2c1]

A mixture of [methyl(diphenyl)silyl]formic acid (151 mg, 0.62 mmol), N-[4-[(3-iodo-7-morpholino-1,6-naphthyridin-5-yl)oxy]cyclohexyl]-5-(trifluoromethyl)pyrimidin-2-amine LFA140 (250 mg, 0.42 mmol), potassium trimethylsilanolate (119 mg, 0.83 mmol), bis(dibenzylideneacetone)palladium(0) (12 mg, 0.02 mmol) and 9,9-dimethyl-4,5-bis(diphenylphosphino)xanthene (12 mg, 0.02 mmol) in toluene toluene (2 mL) was heated 40° C. and stirred for 30 minutes. The mixture was concentrated and the residue was dissolved in MeOH and loaded onto a 5 g SCX cartridge. The column was washed with MeOH and the product was eluted with 2 M NH₃ in MeOH. The NH₃ fraction was concentrated and the residue was dissolved in water and the pH adjusted to ˜pH 6-7 using 1N HCl (aq.) and sat. NaHCO₃ (aq.) solution. The aqueous phase was extracted with CH₂Cl₂ (5×) and CH₂Cl₂/i-PrOH (3:1). The combined organic phases were dried over Na₂SO₄ and concentrated to afford the titled compound (327 mg, crude 151%)

¹H NMR (400 MHz, DMSO-d₆) δ 9.14 (d, J=2.2 Hz, 1H), 8.75 (dd, J=2.3, 0.8 Hz, 1H), 8.61 (s, 1H), 8.57 (s, 1H), 8.19 (d, J=7.8 Hz, 1H), 6.58 (d, J=0.9 Hz, 1H), 5.45-5.36 (m, 1H), 4.04-3.89 (m, 1H), 3.73 (dd, J=5.8, 3.9 Hz, 4H), 3.64-3.53 (m, 4H), 2.24-2.08 (m, 2H), 1.95-1.72 (m, 6H). CO₂H not directly observed.

m/z calcd. C₂₄H₂₅F₃N₆04=518.5. Found [M+H]⁺=519.2.

### 7-Morpholino-5-[4-[[5-(trifluoromethyl)pyrimidin-2-yl]amino]cyclohexoxy]-1,6-naphthyridine-3-carboxamide (LFA142)

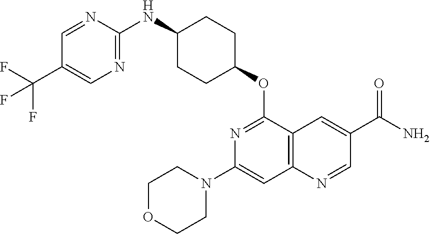 [SMILES: NC(=O)c1cnc2cc(N3CCOCC3)nc(O[C@H]3CC[C@@H](Nc4ncc(C(F)(F)F)cn4)CC3)c2c1]

A mixture of O-(7-aza-1H-benzotriazol-1-yl)-N,N,N′,N′-tetramethyluronium hexafluorophosphate (111 mg, 0.29 mmol), 7-morpholino-5-[4-[[5-(trifluoromethyl)pyrimidin-2-yl]amino]cyclohexoxy]-1,6-naphthyridine-3-carboxylic acid LFA141 (150 mg, 0.29 mmol), ammonium chloride (77 mg, 1.45 mmol) and DIPEA (0.13 mL, 0.72 mmol) in DMF (1 mL) was stirred at room temperature for 18 h. Further O-(7-aza-1H-benzotriazol-1-yl)-N,N,N′,N′-tetramethyluronium hexafluorophosphate (111 mg, 0.29 mmol), DIPEA (0.13 mL, 0.72 mmol) and ammonium chloride (77 mg, 1.45 mmol) were added and the mixture stirred at room temperature for a further 20 h. The mixture was diluted with CH₂Cl₂ and washed with water (3×) and brine, dried over Na₂SO₄, and concentrated. The residue was purified via automated flash chromatography using 0-5% MeOH/CH₂Cl₂ as the mobile phase to afford the titled compound as a yellow solid (56 mg, 37%).

¹H NMR (400 MHz, DMSO-d₆) δ 9.19 (d, J=2.3 Hz, 1H), 8.75 (dd, J=2.3, 0.8 Hz, 1H), 8.64 (br. s, 1H), 8.59 (br. s, 1H), 8.19 (br. s, 1H), 8.13 (d, J=7.8 Hz, 1H), 7.54 (br. s, 1H), 6.58 (d, J=0.9 Hz, 1H), 5.46-5.34 (m, 1H), 4.03-3.91 (m, 1H), 3.79-3.67 (m, 4H), 3.64-3.50 (m, 4H), 2.23-2.11 (m, 2H), 1.91-1.73 (m, 6H).

m/z calcd. C₂₄H₂₆F₃N₇O₃=517.2. Found [M+H]⁺=518.2.

### N-[4-[[7-Morpholino-3-(4H-1,2,4-triazol-3-yl)-1,6-naphthyridin-5-yl]oxy]cyclohexyl]-5-(trifluoromethyl)pyrimidin-2-amine (LFA143)

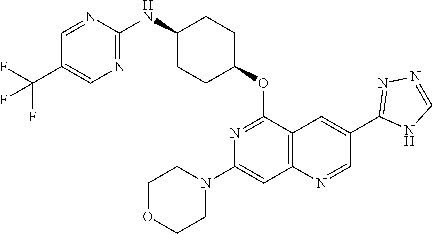 [SMILES: FC(F)(F)c1cnc(N[C@H]2CC[C@@H](Oc3nc(N4CCOCC4)cc4ncc(-c5nnc[nH]5)cc34)CC2)nc1]

7-Morpholino-5-[4-[[5-(trifluoromethyl)pyrimidin-2-yl]amino]cyclohexoxy]-1,6-naphthyridine-3-carboxamide LFA142 (56 mg, 0.11 mmol) was suspended in N,N-dimethylformamide dimethyl acetal (0.72 mL, 5.41 mmol), heated to 90° C. and stirred for 1 h. The mixture was concentrated and the residue was suspended in acetic acid (2 mL) and hydrazine monohydrate (0.053 mL, 1.08 mmol) was added. The mixture was heating to 90° C. and stirred for 1 h. The mixture was concentrated and the residue was suspended/dissolved in CH₂Cl₂ and washed with sat. NaHCO₃ (aq.). The aqueous phase was extracted with 5% MeOH/CH₂Cl₂ (5×). The combined organic phases were dried over Na₂SO₄ and concentrated. The residue was purified via automated flash chromatography using 0-6% MeOH/CH₂Cl₂ as the mobile phase to afford the titled compound as a yellow solid (18 mg, 31%).

¹H NMR (400 MHz, DMSO-d₆) δ 14.32 (s, 1H), 9.39 (d, J=2.2 Hz, 1H), 8.85 (dd, J=2.3, 0.8 Hz, 1H), 8.70-8.49 (m, 3H), 8.20 (d, J=7.7 Hz, 1H), 6.60 (d, J=0.9 Hz, 1H), 5.45-5.35 (m, 1H), 4.03-3.91 (m, 1H), 3.75 (dd, J=5.7, 4.0 Hz, 4H), 3.59-3.51 (m, 4H), 2.26-2.13 (m, 2H), 1.87-1.76 (m, 6H).

m/z calcd. C₂₅H₂₆F₃N₉O₂=541.2. Found [M+H]⁺=542.2.

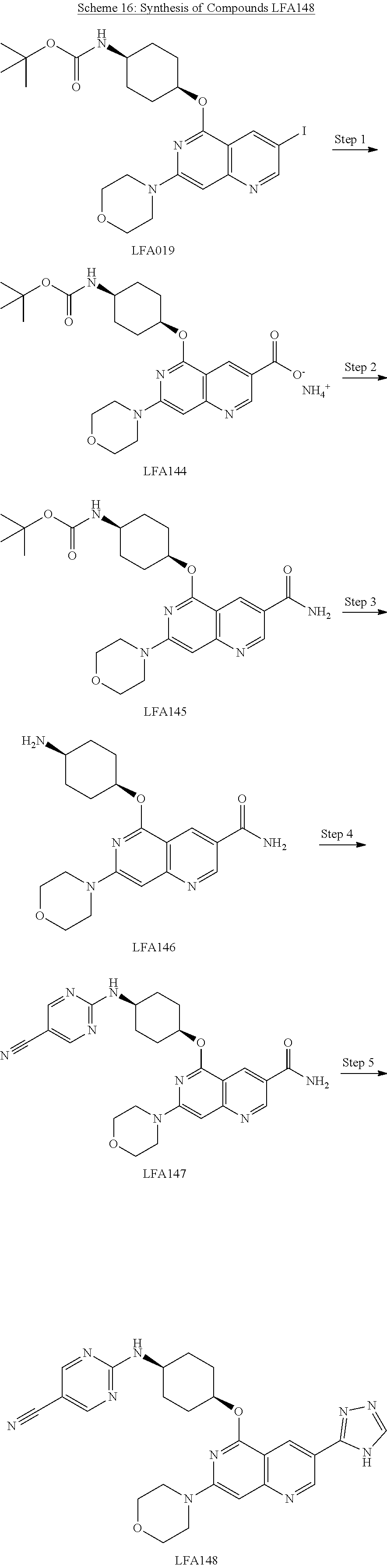

### Step 1

### Ammonium; 5-[4-(tert-butoxycarbonylamino)cyclohexoxy]-7-morpholino-1,6-naphthyridine-3-carboxylate (LFA144)

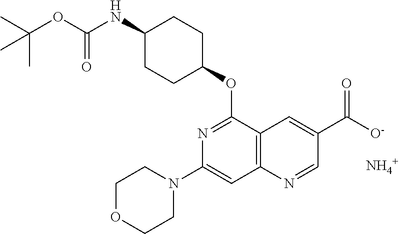 [SMILES: CC(C)(C)OC(=O)N[C@H]1CC[C@@H](Oc2nc(N3CCOCC3)cc3ncc(C(=O)[O-])cc23)CC1 ; [NH4+]]

A mixture of [methyl(diphenyl)silyl]formic acid (131 mg, 0.54 mmol), tert-butyl N-[4-[(3-iodo-7-morpholino-1,6-naphthyridin-5-yl)oxy]cyclohexyl]carbamate LFA019 (200 mg, 0.36 mmol), potassium trimethylsilanolate (103 mg, 0.72 mmol), bis(dibenzylideneacetone)palladium(0) (10 mg, 0.02 mmol) and 9,9-dimethyl-4,5-bis(diphenylphosphino)xanthene (10 mg, 0.02 mmol) in toluene (2 mL) was heated with to 40° C. and stirred for 30 minutes. The mixture was concentrated and the residue was dissolved in MeOH and loaded onto a 5 g SCX cartridge. The column was washed with MeOH and the product was eluted with 2 M NH₃ in MeOH. The NH₃ fraction was concentrated to afford the titled compound as a yellow solid (180 mg, 100%)

¹H NMR (400 MHz, DMSO-d₆) δ 9.16 (d, J=2.2 Hz, 1H), 8.71 (d, J=2.1 Hz, 1H), 6.94 (d, J=7.8 Hz, 1H), 6.57 (s, 1H), 5.38-5.26 (m, 1H), 3.73 (dd, J=6.1, 3.7 Hz, 4H), 3.64-3.53 (m, 4H), 2.18-1.98 (m, 2H), 1.81-1.55 (m, 6H), 1.38 (s, 9H). Analysis is consistent with desired product as the ammonia salt. BocNH and NH₄⁺ not directly observed (likely under water peak).

m/z calcd. C₂₄H₃₂N₄O₆=472.2. Found [M+H]⁺=473.2 (for parent ion).

### tert-Butyl N-[4-[(3-carbamoyl-7-morpholino-1,6-naphthyridin-5-yl)oxy]cyclohexyl]carbamate (LFA145)

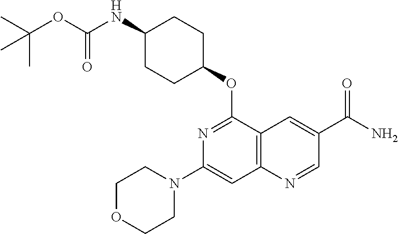 [SMILES: CC(C)(C)OC(=O)N[C@H]1CC[C@@H](Oc2nc(N3CCOCC3)cc3ncc(C(N)=O)cc23)CC1]

A mixture of ammonium; 5-[4-(tert-butoxycarbonylamino)cyclohexoxy]-7-morpholino-1,6-naphthyridine-3-carboxylate LFA144 (180 mg, 0.37 mmol), 0-(7-aza-1H-benzotriazol-1-yl)-N,N,N′,N′-tetramethyluronium hexafluorophosphate (281 mg, 0.74 mmol), ammonium chloride (197 mg, 3.68 mmol) and DIPEA (0.32 mL, 1.84 mmol) in DMF (1 mL) was stirred at room temperature for 17 h. The reaction was diluted with CH₂Cl₂ and washed with water (3×) and brine. The combined organic phases were dried over Na₂SO₄, filtered and concentrated. The residue was purified via automated flash chromatography using 0-8% MeOH/CH₂Cl₂ as the mobile phase to afford the titled compound as a yellow solid (68 mg, 39%).

¹H NMR (400 MHz, DMSO-d₆) δ 9.18 (d, J=2.3 Hz, 1H), 8.70 (dd, J=2.4, 0.8 Hz, 1H), 8.17 (s, 1H), 7.53 (s, 1H), 6.84 (d, J=7.7 Hz, 1H), 6.56 (d, J=0.8 Hz, 1H), 5.38-5.25 (m, 1H), 3.79-3.66 (m, 4H), 3.59-3.52 (m, 4H), 3.47-3.36 (m, 1H), 2.16-1.98 (m, 2H), 1.78-1.60 (m, 6H), 1.38 (s, 9H).

m/z calcd. C₂₄H₃₃N₅O₅=471.2. Found [M+H]⁺=472.2.

### 5-(4-Aminocyclohexoxy)-7-morpholino-1,6-naphthyridine-3-carboxamide (LFA146)

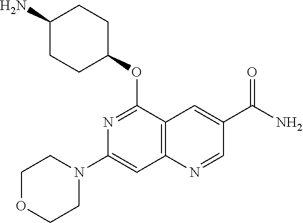 [SMILES: NC(=O)c1cnc2cc(N3CCOCC3)nc(O[C@H]3CC[C@@H](N)CC3)c2c1]

Hydrogen chloride (3.7-4.3 M in 1,4-dioxane) (1.00 mL, 4.00 mmol) was added to a stirred solution of tert-butyl N-[4-[(3-carbamoyl-7-morpholino-1,6-naphthyridin-5-yl)oxy]cyclohexyl]carbamate LFA145 (68 mg, 0.14 mmol) in MeOH (2 mL) and the mixture was stirred at room temperature for 18 h. The reaction was concentrated and the residue was loaded onto a 1 g SCX column. The column was washed with MeOH and the product was eluted with 2 M NH₃ in MeOH. The NH₃ fraction was concentrated to afford the titled compound as a yellow solid (52 mg, 97%).

¹H NMR (400 MHz, DMSO-d₆) δ 9.18 (d, J=2.3 Hz, 1H), 8.71 (dd, J=2.4, 0.8 Hz, 1H), 8.24 (s, 1H), 7.51 (s, 1H), 6.56 (d, J=0.9 Hz, 1H), 5.36-5.26 (m, 1H), 3.73 (dd, J=5.8, 3.9 Hz, 4H), 3.55 (dd, J=5.7, 4.1 Hz, 4H), 2.81-2.72 (m, 1H), 2.14-1.99 (m, 2H), 1.75-1.60 (m, 4H), 1.59-1.46 (m, 2H).

m/z calcd. C₁₉H₂₅N₅O₃=371.2. Found [M+H]⁺=372.2.

### 5-[4-[(5-Cyanopyrimidin-2-yl)amino]cyclohexoxy]-7-morpholino-1,6-naphthyridine-3-carboxamide (LFA147)

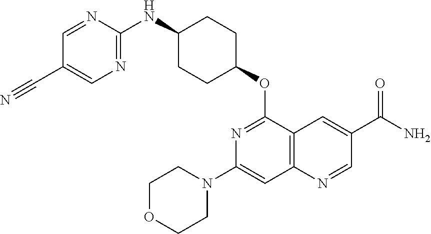 [SMILES: N#Cc1cnc(N[C@H]2CC[C@@H](Oc3nc(N4CCOCC4)cc4ncc(C(N)=O)cc34)CC2)nc1]

A mixture of 5-(4-aminocyclohexoxy)-7-morpholino-1,6-naphthyridine-3-carboxamide LFA146 (53 mg, 0.14 mmol), 2-chloropyrimidine-5-carbonitrile (22 mg, 0.16 mmol), triethylamine (0.06 mL, 0.43 mmol) in EtOH (1 mL) was stirred at room temperature got 18 h. The suspension was concentrated and the residue dissolved in CH₂Cl₂ and washed with water. The organic phase was dried via a hydrophobic frit and concentrated. The residue was purified via automated flash chromatography using 0-7% MeOH/CH₂Cl₂ as the mobile phase to afford the titled compound as a yellow solid (45 mg, 66%).

¹H NMR (400 MHz, DMSO-d₆) δ 9.19 (d, J=2.3 Hz, 1H), 8.78-8.70 (m, 2H), 8.64 (d, J=3.0 Hz, 1H), 8.40 (d, J=7.8 Hz, 1H), 8.19 (s, 1H), 7.55 (s, 1H), 6.58 (d, J=0.8 Hz, 1H), 5.47-5.23 (m, 1H), 4.04-3.90 (m, 1H), 3.78-3.65 (m, 4H), 3.60-3.52 (m, 4H), 2.26-2.08 (m, 2H), 1.92-1.72 (m, 6H).

m/z calcd. C₂₄H₂₆N₈O₃=474.2. Found [M+H]⁺=475.2.

### 2-[[4-[[7-Morpholino-3-(4H-1,2,4-triazol-3-yl)-1,6-naphthyridin-5-yl]oxy]cyclohexyl]amino]pyrimidine-5-carbonitrile (LFA148)

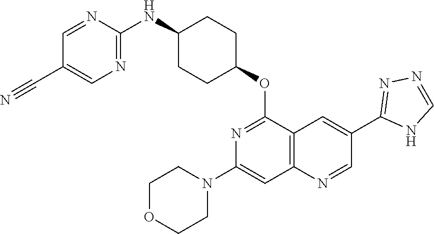 [SMILES: N#Cc1cnc(N[C@H]2CC[C@@H](Oc3nc(N4CCOCC4)cc4ncc(-c5nnc[nH]5)cc34)CC2)nc1]

7-Morpholino-5-[4-(pyrimidin-2-ylamino)cyclohexoxy]-1,6-naphthyridine-3-carboxamide LFA147 (31 mg, 0.07 mmol) was suspended in N,N-dimethylformamide dimethyl acetal (0.63 mL, 4.74 mmol), heated to 90° C. and stirred for 1 h. The mixture was concentrated and the residue was suspended in acetic acid (2 mL) and hydrazine monohydrate (0.023 mL, 0.47 mmol) was added. The mixture was heated to 90° C. and stirred for 2 h. The mixture was concentrated and the residue was suspended/dissolved in CH₂Cl₂ and washed with sat. NaHCO₃ (aq). The aqueous phase was extracted with 5% MeOH/CH₂Cl₂ (5×). The combined organic phases were dried over Na₂SO₄, filtered, and concentrated. The residue was purified via automated flash chromatography using 0-7% MeOH/CH₂Cl₂ as the mobile phase to afford, then via preparative HPLC using 5-95% 0.005 M NH₄OH/MeCN in 0.005 M NH₄OH/H₂O as the mobile phase, and finally by preparative HPLC using acidic conditions and a focused gradient. The appropriate fractions were combined and loaded onto a 1 g SCX column. The column was washed with MeOH followed by 2 M NH₃ in MeOH solution. The NH₃ fraction was concentrated to afford the titled compound as a pale yellow solid (4 mg, 9%).

¹H NMR (400 MHz, DMSO-d₆) δ 9.39 (d, J=2.2 Hz, 1H), 8.84 (dd, J=2.2, 0.9 Hz, 1H), 8.72 (d, J=3.1 Hz, 1H), 8.64 (d, J=3.0 Hz, 1H), 8.57 (s, 1H), 8.46 (d, J=7.7 Hz, 1H), 6.60 (s, 1H), 5.45-5.33 (m, 1H), 4.06-3.89 (m, 1H), 3.78-3.69 (m, 4H), 3.59-3.50 (m, 4H), 2.29-2.11 (m, 2H), 1.91-1.71 (m, 6H). Triazole NH not directly observed.

m/z calcd. C₂₅H₂₆N₁₀O₂=498.2. Found [M+H]⁺=499.4.

Focused HPLC method Preparative HPLC was carried out on a Waters HPLC comprising of a Waters 2767 Sample Manager, Waters 2545 Binary Gradient Module, Waters Systems Fluidics Organiser, Waters 515 ACD pump, Waters 2998 Photodiode Array Detector, using a Waters XBridge Prep OBD C18, 5 μm, 19 mm×50 mm i.d. column and a flow rate of 20 ml/minute. Acidic reverse phase HPLC (water/acetonitrile/0.1% trifluoroacetic acid) using a focused gradient of 5% acetonitrile/95% water (3 min) to 77% acetonitrile/23% water (3.5 min) to 63% acetonitrile/33% water (11 min) ramping quickly to 95% acetonitrile/5% water UV detection e.g. 254 nM is used for the collection of fractions from HPLC.

### Example 3: Syntheses of Compounds

### General Procedure A—Alkoxylation via Aryl Halide Displacement

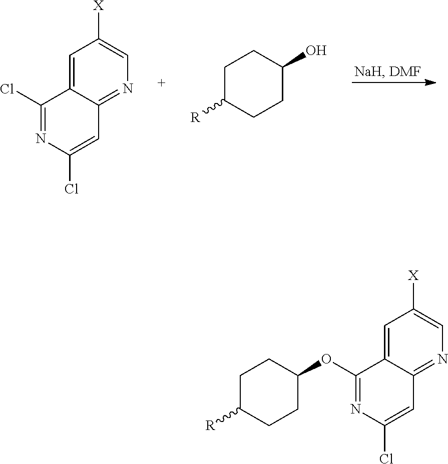

To a cold (0° C.) stirring solution of cyclohexyl alcohol (1.05 eq) in DMF was added 60% sodium hydride (1.4 eq) in one portion. After stirring the resulting yellow mixture for 10 minutes, heteroaryl haldie (1.0 eq) was added in one portion. The resulting dark yellow mixture was stirred cold for 5 minutes then at ambient temperature. After 2 h, brine or saturated aq NaHCO₃ was carefully added then the mixture was extracted with EtOAc (4×), washed with brine (lx), dried over MgSO₄, filtered and concentrated. Purification via automated flash chromatography (50% to 100% DCM in hexanes and/or 5% to 100% EtOAc in hexanes) gave the desired alkoxylated compound. Alternatively, Et₂O was added to the crude residue and sonicated to precipitate the desired alkoxylated compound.

### General Procedure B—Aryl Amination via Morpholine Addition

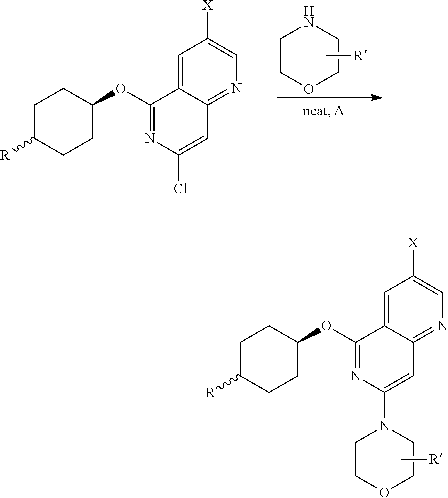

A microwave vial was charged with heteroaryl halide and morpholine (50 eq). The vial was sealed, placed in microwave reactor and eradiated with microwaves for 1-2 h at 150-200° C. The resulting mixture was diluted with EtOAc, concentrated and the resulting residue was purified via automated flash chromatography using EtOAc/hexanes or MeOH/CH₂Cl₂ as the mobile phase to give the desired aminated compound.

### General Procedure C—Hydroxylation via [Pd] C—O Coupling

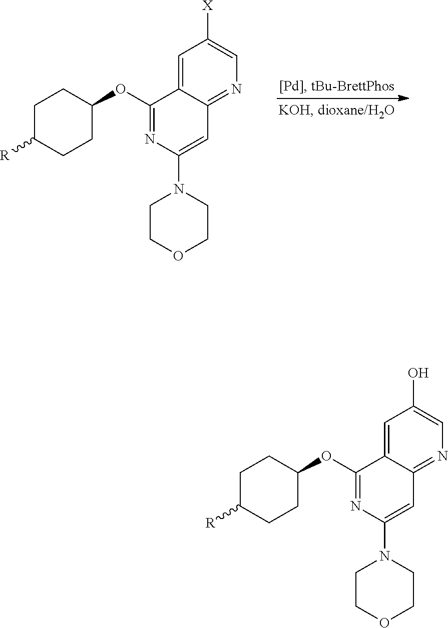

A mixture of heteroaryl halide (1.0 eq), potassium hydroxide (3.1 eq), tBuBrettPhos Palladacycle Generation 3 (6.5%), tBuBrettPhos (6.5%), dioxane and water (3.0 eq) was stirred at 80° C. for 16 h. The reaction mixture was cooled down to ambient temperature, diluted with MeOH and concentrated. The crude product was purified via automated flash chromatography using MeOH/EtOAc as the mobile phase to give the desired hydroxylated compound.

### General Procedure D—Alkoxylation via Alkyl Halide Displacement

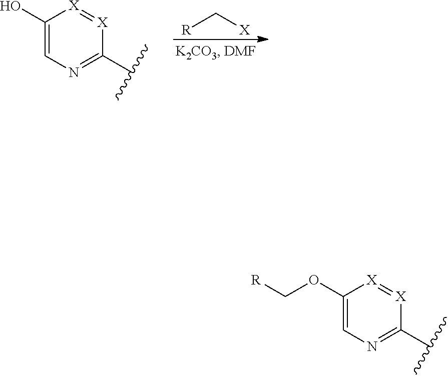

A mixture of hydroxylarene (1.0 eq), alkyl halide (1.3 eq), and potassium carbonate (1.3 eq) in DMF was stirred at 30-60° C. for 2-16 h. The mixture was cooled to ambient temperature, diluted with EtOAc, washed with brine (2×), dried over MgSO₄, filtered and concentrated. The crude product was purified via automated flash chromatography using EtOAc/hexanes as the mobile phase to give the desired alkoxylated arene.

### General Procedure E—Alkoxylation via Activated Hydroxyl Displacement

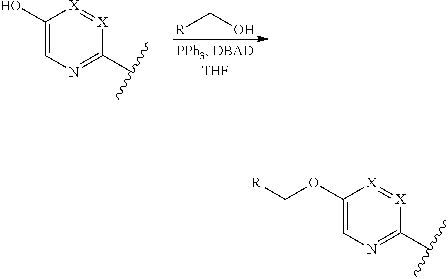

To a cold (0° C.) stirring mixture of hydroxylarene (1.0 eq), alkyl alcohol (1.5 eq), and triphenylphosphine (2.0 eq) in THF was added di-tert-butyl azodicarboxylate (1.6 eq). The mixture was stirred at ambient temperature for 16-24 h and then concentrated. The crude product was purified via automated flash chromatography using EtOAc/hexanes and/or MeOH/CH₂Cl₂ as the mobile phase to give the desired alkoxylated arene.

### General Procedure F—Imidazole Formation

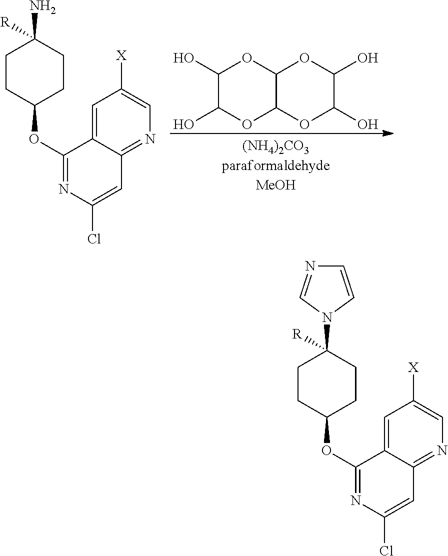

A mixture of an appropriate amine (1.0 eq), ammonium carbonate (1.5 eq), paraformaldehyde (3.3 eq) and hexahydro-[1,4]dioxino[2,3-b][1,4]dioxine-2,3,6,7-tetraol (1.2 eq) in MeOH (0.05M) was stirred at RT overnight. An additional portion of ammonium carbonate (1.5 eq), paraformaldehyde (3.3 eq) and hexahydro-[1,4]dioxino[2,3-b][1,4]dioxine-2,3,6,7-tetraol (1.2 eq) was added and the reaction was stirred at room temperature for another 20 h. The reaction mixture was concentrated and the crude material was partitioned between EtOAc and sat. NaHCO₃ aq. The combined organic layers was dried over Na₂SO₄ and concentrated. The crude product was purified via automated flash chromatography (2% to 15% MeOH in DCM) to give the desired imidazole.

### General Procedure G—Amination Via Aryl Halide Displacement

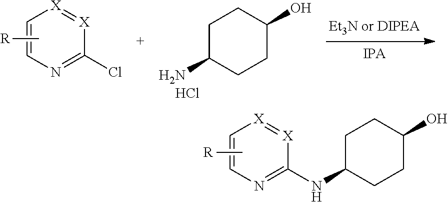

A microwave vial was charged with the appropriate aryl chloride (1.0 eq), (1s,4s)-4-aminocyclohexan-1-ol hydrochloride (2.0 eq), triethylamine or di-isopropylethylamine (3.0 eq), and isopropanol. Alternatively, a microwave vial was charged with the appropriate aryl chloride (1.0 eq), (1s,4s)-4-aminocyclohexan-1-ol (2-4 eq), and isopropanol. The vial was sealed, placed in a microwave reactor and eradiated with microwaves for 1-5 h at 130-180° C. The resulting mixture was concentrated and the crude product was purified via automated flash chromatography using EtOAc/hexanes as the mobile phase to give the desired aryl alcohol.

### General Procedure H—Aryl Amination via Amine Addition

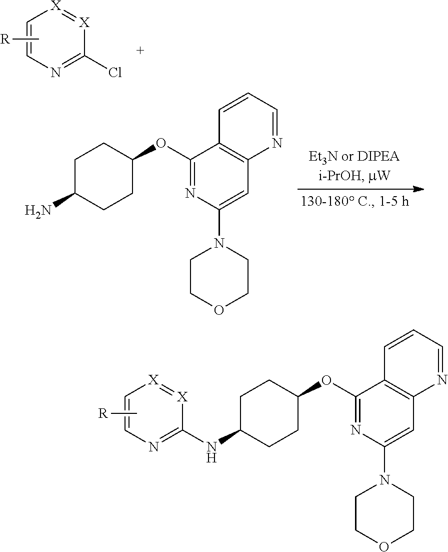

A sealed microwave vial containing aryl chloride (1.1-3.0 eq), 2 (1.0 eq), base (2-8 eq) and i-PrOH was eradiated with microwaves for 1-5 h at 130-180° C. The resulting mixture was diluted with MeOH and concentrated. The crude product was purified via automated flash chromatography using MeOH/CH₂Cl₂ as the mobile phase or via prep HPLC to give the desired aminated compound.

### General Procedure I—Aryl Amination Via Pd₂(Dba)₃ Coupling

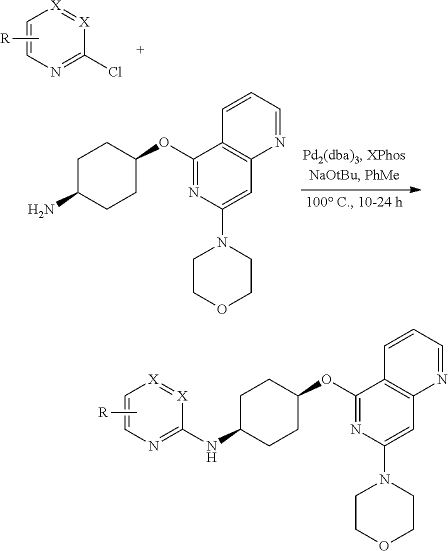

A mixture of 2 (1.0 eq), the appropriate heteroaryl halide (0.8 eq), sodium tert-butoxide (3.0 eq), XPhos (0.2 eq) and Pd₂(dba)₃ (0.1 eq) in toluene (0.13M) under argon was heated at 100° C. for 10-24 h. The crude product was purified via prep HPLC to give the desired aminated product.

### General Procedure J—Aryl Amination Via [Pd(Allyl)Cl]₂ Coupling

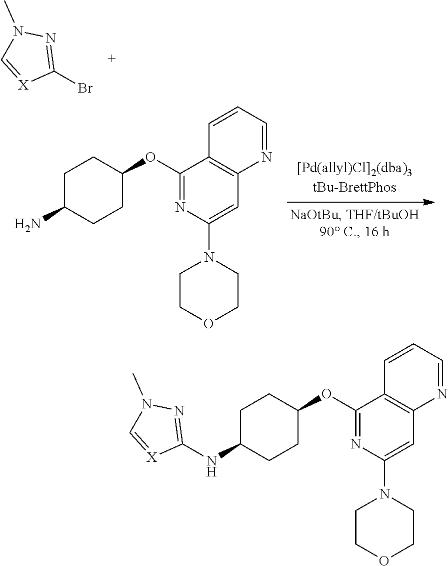

A mixture of compound 2 (1.0 eq), the appropriate aryl bromide (1.2 eq), allyl Pd(II)chloride dimer (0.05 eq), tert-butyl BrettPhos (0.1 eq), and 2M sodium tert-butoxide in THF (2.0 eq) in t-BuOH (O2M) was heated under argon at 90° C. overnight. The crude product was purified via prep HPLC to the desired aminated compound.

### General Procedure K—Aryl Amination Via Pd(OAc)₂ Coupling

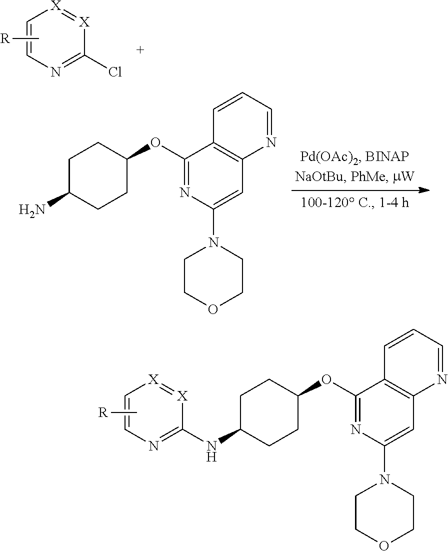

A sealed microwave vial containing aryl chloride (1.1-3.0 eq), 2 (1.0 eq), Pd(OAc)₂ (0.2 eq), rac-BINAP (0.2 eq), sodium tert-butoxide (3.0 eq) and toluene was eradiated with microwaves for 1-4 h at 100-120° C. The resulting mixture was diluted with toluene, passed through a bed of Celite and concentrated. The crude product was purified via automated reverse phase flash chromatography using ACN/H₂O (0.1% TFA) as the mobile phase to give the desired aminated compound.

### General Procedure L—Aryl Amination Via [Pd] Coupling

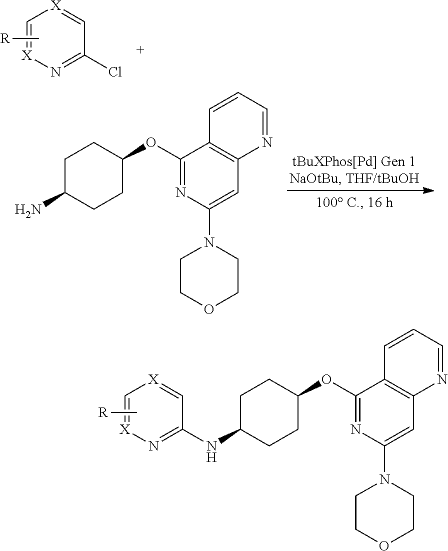

A mixture of compound 2 (1.0 eq), the appropriate aryl chloride (1.2 eq), t-BuXPhos Palladacycle Gen1 (0.1 eq), and 2M sodium tert-butoxide in THF (3.0 eq) in t-BuOH (0.15M) was heated under argon at 100° C. overnight. The crude product was purified via prep HPLC to the desired aminated compound.

### General Procedure M—Amination Via [Cu] C—N Coupling

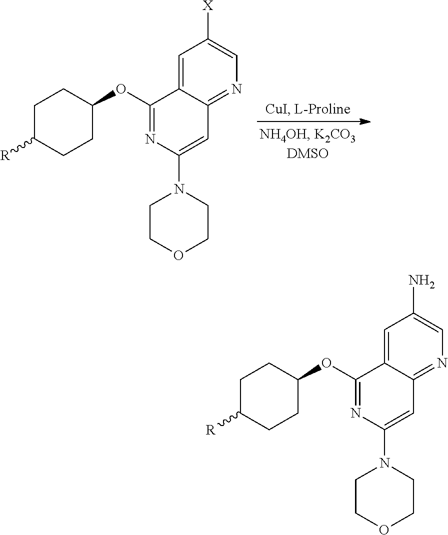

A mixture of the appropriate bromide or iodide (1.0 eq), 14.5M ammonium hydroxide (1.5-3.0 eq), CuI (0.2 eq). L-proline (0.4 eq), potassium carbonate (1.5 eq) in DMSO (O2M to 0.4M) was heated at 90° C. to 100° C. for 12 h to 24 h. The reaction mixture was quenched with sat. NaHCO₃ aq. and extracted with EtOAc. The combined organic layers was dried over Na₂SO₄ and concentrated. Purification via automated flash chromatography (2% to 15% MeOH in DCM) or via prep HPLC gave the desired amino compound.

### General Procedure N—Sulfonamide Formation Via Amine Addition to Sulfonyl Chlorides

A solution of sulfonyl chloride (1.2 eq) in CH₂Cl₂ was slowly added to a solution of amine (1.0 eq) and DIPEA (2.0 eq) in CH₂Cl₂ or a solution of amine (1.0 eq) in pyridine. After stirring for 30-60 minutes, the mixture was concentrated and purified via automated flash chromatography using MeOH/CH₂Cl₂ as the mobile phase to give the desired aminated compound.

### General Procedure O—Amide Formation Via HATU Coupling

To a stirring mixture of a carboxylic acid (1.3 or 1.0 eq) and 0-(7-azabenzotriazol-1-yl)-N,N,N′,N′-tetramethyluronium hexafluorophosphate (1.6 eq wrt acid) in DMF was added DIPEA (4.0 eq wrt acid). After stirring for 1-5 minutes, an amine (1.0 or 1.3 eq) was added and stirring continued for 1-16 h. The mixture was diluted with EtOAc, washed with H₂O, saturated aq NaHCO₃, and brine, dried over MgSO₄, filtered and concentrated. The crude product was purified via automated flash chromatography (normal or reverse phase) using MeOH/CH₂Cl₂ or ACN/H₂O (0.1% TFA) as the mobile phase to give the desired aminated compound.

### General Procedure P—Amination Via Alkyl Halide Displacement

To a cold (0° C.) stirring solution of amine or amide (1.0 eq) in DMF was added 60% sodium hydride (1.2-2.5 eq)—immediate bubbling. The resulting mixture was stirring cold for 5 min then at ambient temperature for 10-20 min at which point the appropriate alkyl halide (1.0-1.5 eq) was added. After stirring for 0.5-18 h, the mixture was diluted with EtOAc, washed with brine (2×), dried over MgSO₄, filtered and concentrated. The crude product was purified via automated flash chromatography using MeOH/CH₂Cl₂ as the mobile phase to give the desired aminated compound.

### General Procedure Q—Amide Formation Via Amine Addition to Acid Chlorides

A solution of acid chloride (1.2 eq) in CH₂Cl₂ was slowly added to a solution of amine (1.0 eq) and DIPEA (2.0 eq) in CH₂Cl₂. After stirring for 30-60 minutes, the mixture was concentrated and purified via automated flash chromatography using MeOH/CH₂Cl₂ as the mobile phase to give the desired aminated compound.

### General Procedure R—Aryl Alkoxylation Via Alcohol Addition

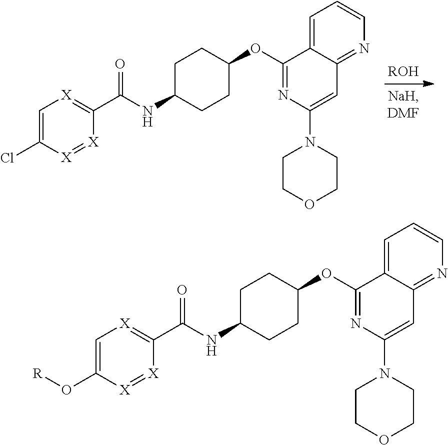

To a mixture of an appropriate Aryl chloride (1.0 eq.), and an appropriate ROH (1.1 eq.) in DMF (0.06 M) was added NaH (7.0 eq.) at 5° C. The reaction mixture was then stirred at 50° C. or 70° C. for 30 min to 5 h. The reaction mixture was quenched with water and extracted with EtOAc. The organic layer was washed with brine, dried over Na₂SO₄, filtered and concentrated. The remaining residue was purified by automated flash chromatography (DCM:MeOH) to give the desired product.

### General Procedure S—Urea formation via Amine Addition to Isocyanates

A solution of isocyanate (1.2 eq) in CH₂Cl₂ was slowly added to a solution of amine (1.0 eq) and DIPEA (2.0 eq) in CH₂Cl₂. After stirring for 30-60 minutes, the mixture was concentrated and purified via automated flash chromatography using MeOH/CH₂Cl₂ as the mobile phase to give the desired aminated compound.

### General Procedure T—Arylation via Pd C—C Coupling

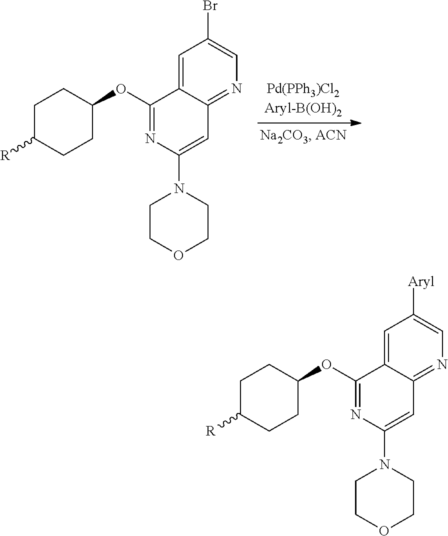

A mixture of aryl bromide (1.0 eq), the appropriate boronic acid (1.5 eq), Pd(PPh₃)₂Cl₂ (0.12 eq), 2M Na₂CO₃ (4.0 to 5.0 eq) in ACN (0.08M) under Argon was heated via microwave at 120-130° C. for 1-2 h. The crude product was purified via prep HPLC gave the desired arylated compound.

### General Procedure U—Arylation via Pd C—C Coupling

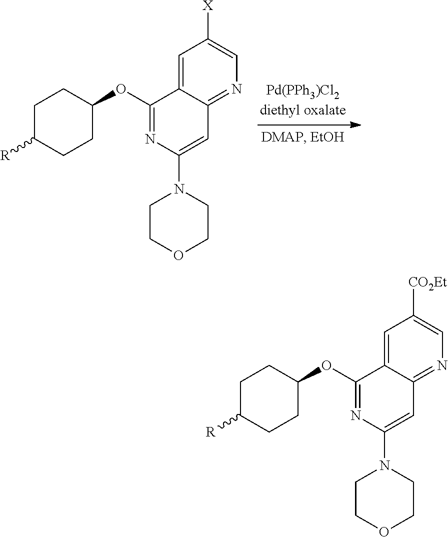

A mixture of aryl halide (0.11 mmol, 50 mg), diethyl oxalate (3.0 eq), Pd(PPh₃)₂Cl₂ (4 mol %), and DMAP (5.5 eq) in EtOH (0.5M) under argon was heated via microwave at 140° C. for 20 min. An additional portion of Pd(PPh₃)₂Cl₂ (4 mol %) and diethyl oxalate (3.0 eq) was added and the reaction was heated via microwave at 140° C. for 40 min. The reaction was quenched with Na₂CO₃ and extracted with EtOAc. The combined organic layers was dried over Na₂SO₄ and concentrated. The crude product was purified via automated flash chromatography (MeOH:DCM or EtOAc:hexanes) provided the desired carboxylated compound.

### General Procedure V—Hydroxamic Acid formation via CDI Coupling

To a solution of a carboxylic acid (1.0 eq) in DMF (0.1M) was added CDI (6.0 eq). The reaction mixture was stirred at RT for 1 h to 20 h. The appropriate hydroxylamine (10 to 20 eq) was then added and the reaction was stirred at RT overnight. The crude product was purified via prep HPLC to give the desired hydroxamic acid compound.

### General Procedure W—Acyl Sulfonamide Formation Via CDI Coupling

To a carboxylic acid (1.0 eq) in DMF (0.07M to 0.14M) was added CDI (4.0 eq). The reaction mixture was stirred at RT for 10-40 min. The appropriate sulfonamide (10 eq) was then added followed by DMAP (cat.). The reaction mixture was heated at 65° C. for 20 h. An additional portion of the appropriate sulfonamide (10 eq) may be added along with further heating at 65° C. for another 24 h to drive the reaction to completion. The crude product was purified via prep HPLC to give the desired sulfonamide compound.

### General Procedure X—Hydrolysis with TFA

A solution of either tert-butylester or tert-butylcarbamate in CH₂Cl₂ was treated with excess trifluoroacetic acid. After stirring for 1 h, the mixture was concentrated and purified via automated flash chromatography (normal or reverse phase) using MeOH/CH₂Cl₂ or ACN/H₂O (0.1% TFA) as the mobile phase to give the desired acid or amine compound, respectively.

### 3-Bromo-5,7-dichloro-1,6-naphthyridine (1a)

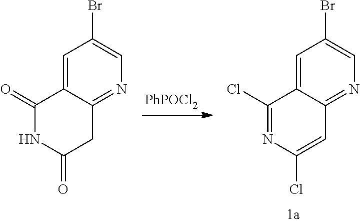 [SMILES: "PhPOCl2" ; O=C1Cc2ncc(Br)cc2C(=O)N1 ; Clc1cc2ncc(Br)cc2c(Cl)n1]

A mixture of commercially available 3-bromo-1,6-naphthyridine-5,7(6H,8H)-dione (5 g, 20.74 mmol) and PhPOCl₂ (50 mL) was stirred at 130° C. for 3 h. After cooling to room temperature, the reaction mixture was poured onto ice-cold water (300 mL) and the product was extracted with EtOAc (2×300 mL). The combined organic layers was washed with sat. NaHCO₃ followed by brine, and dried over Na₂SO₄. After filtering and concentrating, the remaining residue was purified via automated flash chromatography (5% to 100% EtOAc in hexanes) to give 1a as pale yellow solid (2.3 g, 40%).

### 5,7-Dichloro-3-iodo-1,6-naphthyridine (1c)

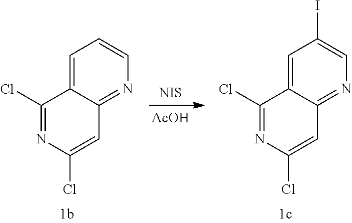

A mixture of commercially available compound 1b (1.0 eq) and N-iodosuccinimide (1.1 eq) in AcOH (0.25M) was heated at 100° C. for 4 h. The reaction was quenched with sat NaHCO₃ and the aqueous layer was extracted with EtOAc. Purification via automated flash chromatography (10% to 60% EtOAc in hexanes) gave compound 1c (yield 36%).

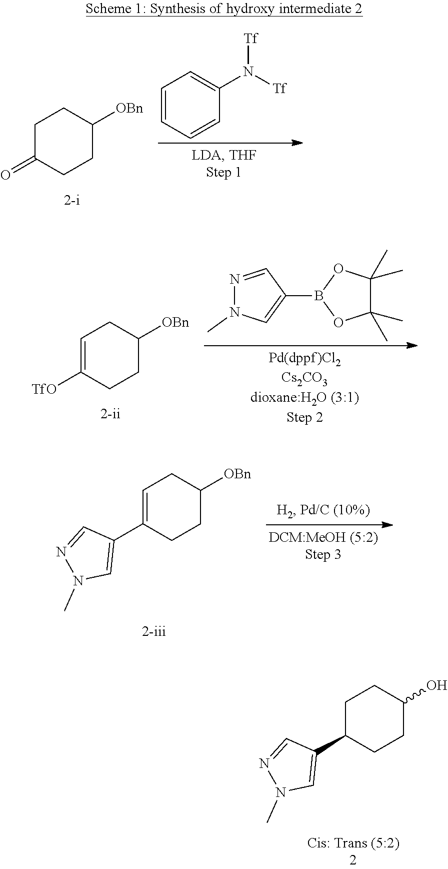

### Step 1

A solution of LDA (2.7 mL, 5.38 mmol) in THF (20 mL) was cooled to −78° C. and a solution of 4-(benzyloxy)cyclohexanone 2-i (1 g, 4.90 mmol) in THF (3 mL) was slowly added. The mixture was stirred at −78° C. for 2 h before adding N-Phenyl-bis(trifluoromethanesulfonimide) (1.75 g, 4.90 mmol) in one portion. The reaction mixture was then allowed to warm gradually to 0° C. and stirred at 0° C. for an additional of 1 h. A solution of EtOAc:Hex (2:8) was added and the organic layer was washed with water followed by brine, dried over Na₂SO₄, filtered and concentrated. The remaining residue was purified via automated flash chromatography (1% to 20% EtOAc in hexanes) to give the intermediate 2-ii as clear oil (870 mg, 48%).

### Step 2

A suspension of 2-ii (800 mg, 2.38 mmol), 1-methylpyrazole-4-boronic acid pinacol ester (495 mg, 2.38 mmol), Pd(dppf)Cl₂ (174 mg, 0.24 mmol) and Cs₂CO₃ (2.33 g, 7.1 mmol) in a mixture of dioxane:water (3:1, 20 mL) was heated at 85° C. for 1 h under N₂ atmosphere. After cooling to room temperature, the reaction mixture was taken up into EtOAc and sat. NaHCO₃. The organic layer was dried over Na₂SO₄, filtered and concentrated. The remaining residue was purified via automated flash chromatography (1% to 50% EtOAc in hexanes) to give the intermediate 2-iii as pale yellow solid (636 mg, 99%).

### Step 3

A suspension of 2-iii (640 mg, 2.37 mmol) and 10% Pd/C (220 mg, 0.21 mmol) in DCM:MeOH (5:2, 42 mL) was stirred under H₂ atmosphere for 2 days. More 10% Pd/C (220 mg, 0.21 mmol) was added and the reaction mixture stirred under H₂ atmosphere for 2 more days. The reaction mixture was filtered through celite, washed thoroughly with MeOH, and concentrated under reduced pressure. The remaining residue was purified via automated flash chromatography (10% to 100% EtOAc in hexanes) to give hydroxy intermediate 2 as a mixture of two isomers (5:2, 320 mg, 75%).

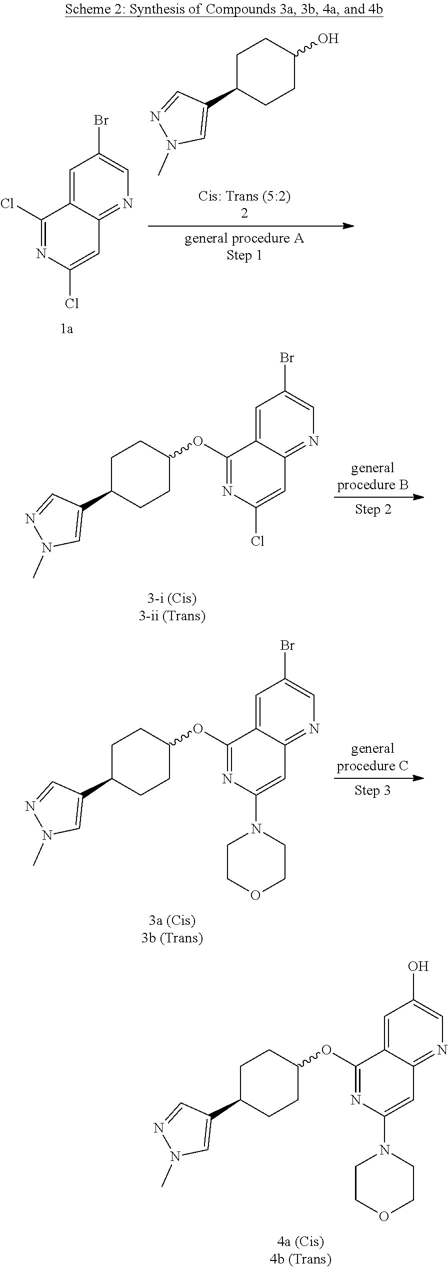

Prepared according to general procedure A to give the crude product as a mixture of cis/trans isomers (3:2). The two isomers were isolated by automated reverse phase flash chromatography (ACN/H₂O+TFA). The purified fractions of each isomer were combined and freeze dried to afford the cis-isomer (3-i) as yellow solid (36%) and trans-isomer (3-ii) as light brown solid (24%).

### Step 2

Prepared according to general procedure B to give compounds 3a or 3b.

### Step 3

Prepared according to general procedure C to give compounds 4a or 4b.

### 4a: 5-(((1s,4s)-4-(1-methyl-1H-pyrazol-4-yl)cyclohexyl)oxy)-7-morpholino-1,6-naphthyridin-3-ol

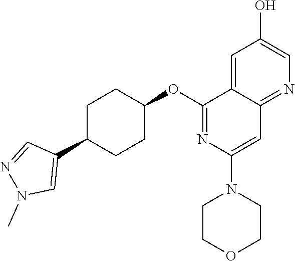 [SMILES: Cn1cc([C@H]2CC[C@@H](Oc3nc(N4CCOCC4)cc4ncc(O)cc34)CC2)cn1]

Compound 4a was synthesized from 3a according to Scheme 2. ¹H NMR (400 MHz, DMSO-d₆) δ 9.99 (s, 1H), 8.51 (d, J=2.9 Hz, 1H), 7.57 (d, J=2.9 Hz, 1H), 7.50 (s, 1H), 7.32 (s, 1H), 6.47 (s, 1H), 5.43 (s, 1H), 3.78 (s, 3H), 3.74 (t, J=4.9 Hz, 4H), 3.39 (t, J=4.9 Hz, 4H), 2.70-2.55 (m, 1H), 2.07 (d, J=10.6 Hz, 2H), 1.87-1.67 (m, 6H). ¹³C NMR (101 MHz, DMSO-d₆) δ 157.25, 154.46, 149.45, 148.90, 147.52, 136.32, 127.31, 126.53, 112.42, 109.15, 92.57, 69.81, 65.89, 45.87, 38.34, 32.32, 29.06, 28.49.

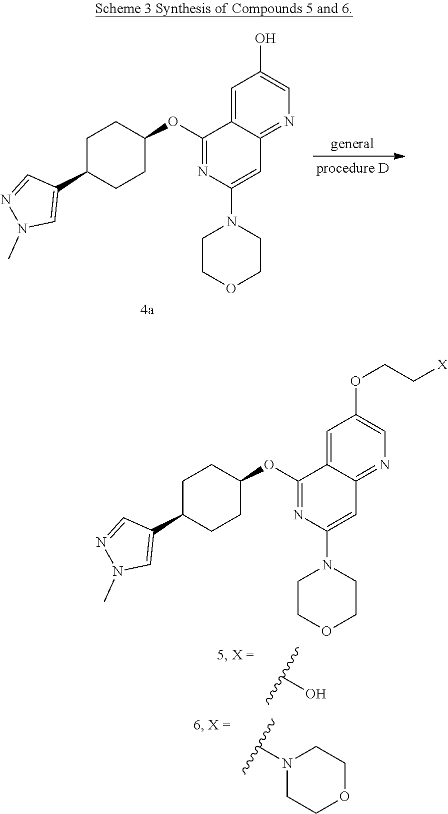

### 5:2-((5-(((1s,4s)-4-(1-methyl-1H-pyrazol-4-yl)cyclohexyl)oxy)-7-morpholino-1,6-naphthyridin-3-yl)oxy)ethan-1-ol

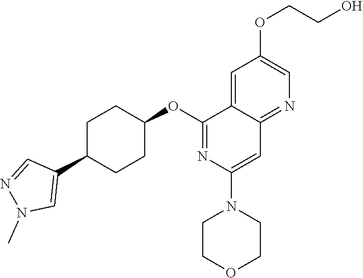 [SMILES: Cn1cc([C@H]2CC[C@@H](Oc3nc(N4CCOCC4)cc4ncc(OCCO)cc34)CC2)cn1]

Prepared according to general procedure D starting with 4a and 2-bromoethanol to afford the titled compound in 45% yield. ¹H NMR (400 MHz, DMSO-d₆) δ 8.63 (d, J=3.0 Hz, 1H), 7.61 (d, J=3.0 Hz, 1H), 7.51 (s, 1H), 7.31 (s, 1H), 6.52 (s, 1H), 5.41 (s, 1H), 4.93 (bs, 1H), 4.11 (t, J=4.8 Hz, 2H), 3.81-3.71 (m, 9H), 3.43 (t, J=4.9 Hz, 4H), 2.66-2.57 (m, 1H), 2.13-2.01 (m, 2H), 1.86-1.69 (m, 6H). ¹³C NMR (101 MHz, DMSO-d₆) δ 157.53, 154.98, 150.33, 147.98, 136.40, 127.42, 126.41, 113.43, 110.74, 108.35, 92.48, 70.53, 70.27, 65.88, 59.47, 45.70, 38.34, 32.20, 28.97, 28.67.

### 6: 4-(5-(((1s,4s)-4-(1-methyl-1H-pyrazol-4-yl)cyclohexyl)oxy)-3-(2-morpholinoethoxy)-1,6-naphthyridin-7-yl)morpholine

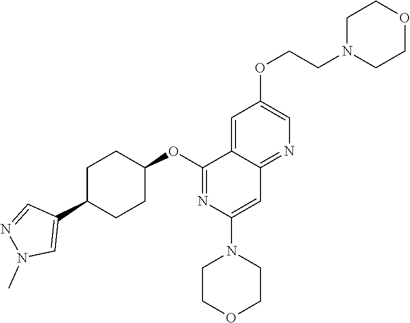 [SMILES: Cn1cc([C@H]2CC[C@@H](Oc3nc(N4CCOCC4)cc4ncc(OCCN5CCOCC5)cc34)CC2)cn1]

Prepared according to general procedure D starting with 4a and 4-(2-bromoethyl)morpholine to afford the titled compound in 55% yield. ¹H NMR (400 MHz, DMSO-d₆) δ 8.62 (d, J=3.0 Hz, 1H), 7.61 (d, J=3.0 Hz, 1H), 7.51 (s, 1H), 7.31 (s, 1H), 6.52 (s, 1H), 5.41 (s, 1H), 4.21 (t, J=5.6 Hz, 2H), 3.82-3.70 (m, 7H), 3.55 (t, J=4.6 Hz, 4H), 3.43 (t, J=4.9 Hz, 4H), 2.72 (t, J=5.6 Hz, 2H), 2.69-2.58 (m, 1H), 2.48-2.43 (m, 4H), 2.14-1.99 (m, 2H), 1.89-1.67 (m, 6H). ¹³C NMR (101 MHz, DMSO-d₆) δ 157.54, 154.98, 150.33, 150.07, 148.05, 136.38, 127.43, 126.35, 110.75, 108.31, 92.47, 70.61, 66.14, 65.88, 56.81, 53.60, 45.69, 38.34, 32.11, 28.93, 28.63.

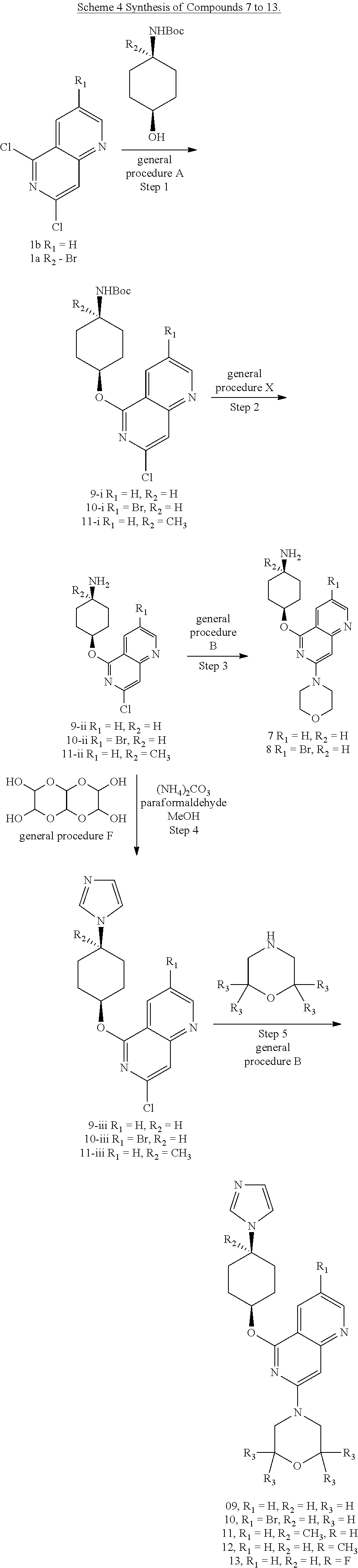

### Step 1

Prepared according to general procedure A starting with 1a or 1b and tert-butyl ((1s,4s)-4-hydroxycyclohexyl)carbamate or tert-butyl ((1s,4s)-4-hydroxy-1-methylcyclohexyl)carbamate 2-chloro-5-isopropylpyrimidine to afford compounds 9-i, 10-i, or 11-i, respectively (yield 64-96%).

### Step 2

Prepared according to general procedure X starting with 9-i, 10-i, or 11-i to afford compounds 9-ii, 10-ii, or 11-ii, respectively (yield quantitative).

### Step 3

Compound 9-ii, and 10-ii (1.0 eq) was combined with morpholine (14.5-18.5 eq) and heated via microwave at 160-180° C. for 40-80 min. The reaction mixture was concentrated and the crude material was purified via automated flash chromatography (C-18, 10% to 100% ACN in water with 0.1% TFA). The collected fractions was concentrated then quenched with sat. NaHCO₃. The resultant precipitate was filtered and dried. Alternatively, the reaction mixture was diluted with water and the resultant precipitate was filtered, washed with water and dried to give compound 7 or 8 (yield 48-97%).

### Step 4

Prepared according to general procedure F starting with 9-ii, 10-ii, or 11-ii to afford compounds 9-iii, 10-iii, or 11-iii, respectively (yield 13-40%).

### Step 5

Prepared according to general procedure B starting with 9-iii, 10-iii, or 11-iii and the appropriate amine to afford compounds 9-13.

### 10 4-(5-(((1s,4s)-4-(1H-imidazol-1-yl)cyclohexyl)oxy)-3-bromo-1,6-naphthyridin-7-yl)morpholine

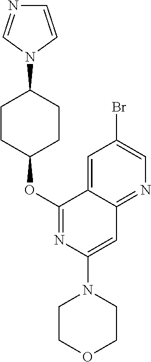 [SMILES: Brc1cnc2cc(N3CCOCC3)nc(O[C@H]3CC[C@@H](n4ccnc4)CC3)c2c1]

Compound 10-iii (1.0 eq) was combined with morpholine (18.5 eq) and heated via microwave at 180° C. for 30 min. The reaction was partitioned between sat. NaHCO₃ and water. The combined organic layers was dried over Na₂SO₄ and concentrated. Purification via automated flash chromatography (2% to 15% MeOH in DCM) gave the titled compound (yield 83%). ¹H NMR (400 MHz, DMSO) δ 8.84 (d, J=2.4 Hz, 1H), 8.59 (d, J=2.4 Hz, 1H), 7.79 (s, 1H), 7.35 (s, 1H), 6.91 (s, 1H), 6.56 (s, 1H), 5.46 (s, 1H), 4.21 (t, J=11.6 Hz, 1H), 3.78-3.71 (m, 4H), 3.56-3.49 (m, 4H), 2.25-2.07 (m, 4H), 1.96-1.80 (m, 4H). m/z calcd. for C₂₁H₂₄BrN₅O₂=457.1. Found [M+H]⁺=458.2.

### 12 4-(5-(((1s,4s)-4-(1H-imidazol-1-yl)cyclohexyl)oxy)-1,6-naphthyridin-7-yl)-2,2,6,6-tetramethylmorpholine

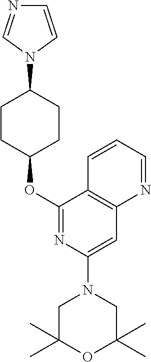 [SMILES: CC1(C)CN(c2cc3ncccc3c(O[C@H]3CC[C@@H](n4ccnc4)CC3)n2)CC(C)(C)O1]

A mixture of 9-iii (1.0 eq) and 2,2,6,6-tetramethylmorpholine (1.2 eq) in NMP (O2M) was heated via microwave at 200° C. for 4 h. The reaction mixture was quenched with water and extracted with EtOAc. The combined organic layers was dried over Na₂SO₄ and concentrated. Purification via prep HPLC gave the titled compound (yield 13%). ¹H NMR (400 MHz, MeOD) δ 9.13 (s, 1H), 9.04 (d, J=7.9 Hz, 1H), 8.71 (d, J=5.9 Hz, 1H), 7.88 (s, 1H), 7.64 (s, 1H), 7.38 (dd, J=7.9, 5.8 Hz, 1H), 6.46 (s, 1H), 5.69-5.64 (m, 1H), 4.64-4.54 (m, 1H), 3.74 (s, 4H), 2.49-2.26 (m, 2H), 2.24-2.15 (m, 2H), 2.12-1.99 (m, 2H), 1.31 (s, 12H). ¹³C NMR (101 MHz, MeOD) δ 160.00, 158.81, 147.02, 145.98, 142.66, 133.74, 120.40, 119.76, 114.75, 111.67, 80.63, 72.09, 70.93, 58.44, 53.30, 28.00, 27.50, 27.28. m/z calcd. for C₂₅H₃₃N₅O₂=435.3. Found [M+H]⁺=436.3.

### 13 4-(5-(((1s,4s)-4-(1H-imidazol-1-yl)cyclohexyl)oxy)-1,6-naphthyridin-7-yl)-2,2,6,6-tetrafluoromorpholine

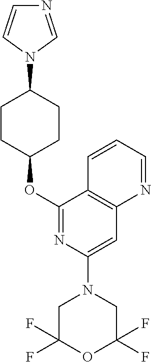 [SMILES: FC1(F)CN(c2cc3ncccc3c(O[C@H]3CC[C@@H](n4ccnc4)CC3)n2)CC(F)(F)O1]

To a mixture of 9-iii (0.061 mmol, 20 mg), 2,2,6,6-tetrafluoromorpholine (1.5 eq), XPhos (0.2 eq), and sodium tert-butoxide (3.0 eq) in toluene under argon was added Pd₂(dba)₃ (0.1 eq). The reaction was heated at 100° C. for 2 h. Purification via prep HPLC gave the titled compound (yield 46%). ¹H NMR (400 MHz, DMSO) δ 9.26 (s, 1H), 8.91 (dd, J=4.4, 1.8 Hz, 1H), 8.64 (dd, J=8.2, 1.8 Hz, 1H), 7.99 (s, 1H), 7.76 (s, 1H), 7.36 (dd, J=8.2, 4.4 Hz, 1H), 6.97 (s, 1H), 5.76 (s, 1H), 5.63-5.57 (m, 1H), 4.57-4.44 (m, 5H), 2.28-2.02 (m, 6H), 1.98-1.85 (m, 2H). ¹³C NMR (101 MHz, DMSO) δ 159.07, 155.25, 154.89, 153.85, 134.55, 134.16, 121.12, 121.05, 120.41, 119.33, 109.95, 93.15, 69.67, 57.98, 46.47, 28.38, 27.75. m/z calcd. for C₂₁H₂₁F4N₅O₂=451.2. Found [M+H]⁺=452.2.

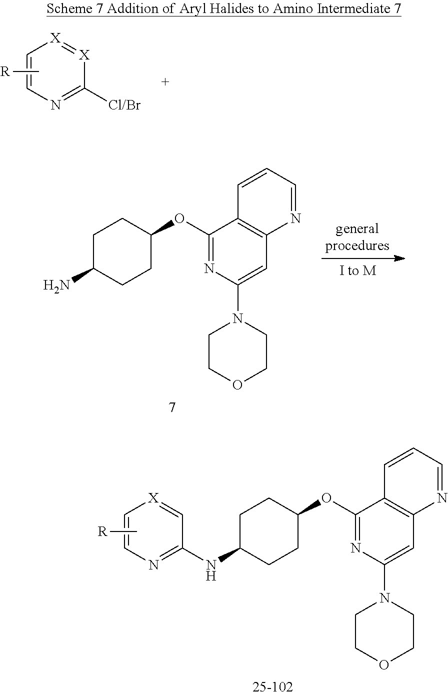

### 2-((2-Chloropyrimidin-5-yl)oxy)-N,N-dimethylacetamide (54-i)

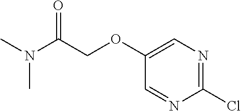

Prepared according to general procedure D starting with 2-chloropyrimidin-5-ol and 2-chloro-N,N-dimethylacetamide to afford the titled compound in 97% yield. ¹H NMR (400 MHz, DMSO-d₆) δ 8.50 (s, 2H), 5.08 (s, 2H), 2.96 (s, 3H), 2.85 (s, 3H). m/z calcd. for C₈H₁₀ClN₃O₂=215.1. Found [M+H]⁺=216.1.

### N,N-Dimethyl-2-((2-(((1s,4s)-4-((7-morpholino-1,6-naphthyridin-5-yl)oxy)cyclohexyl)amino)pyrimidin-5-yl)oxy)acetamide (54)

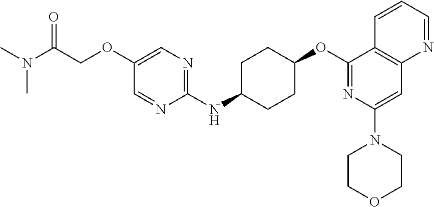 [SMILES: CN(C)C(=O)COc1cnc(N[C@H]2CC[C@@H](Oc3nc(N4CCOCC4)cc4ncccc34)CC2)nc1]

Prepared according to general procedure H starting with 54-i and intermediate 7 to afford the titled compound in 29% yield. ¹H NMR (400 MHz, DMSO-d₆) δ 8.80 (dd, J=4.4, 1.9 Hz, 1H), 8.37 (dd, J=8.3, 1.9 Hz, 1H), 8.09 (s, 2H), 7.22 (dd, J=8.2, 4.3 Hz, 1H), 6.75 (d, J=7.9 Hz, 1H), 6.53 (s, 1H), 5.41 (s, 1H), 4.77 (s, 2H), 3.86-3.67 (m, 5H), 3.50 (t, J=4.9 Hz, 4H), 2.96 (s, 3H), 2.84 (s, 3H), 2.11-2.06 (m, 2H), 1.82-1.71 (m, 6H). ¹³C NMR (100 MHz, DMSO-d₆) δ 167.63, 158.89, 158.20, 156.95, 155.71, 155.31, 146.77, 145.40, 132.74, 118.30, 109.09, 92.41, 70.61, 67.88, 66.34, 49.07, 45.84, 35.94, 35.37, 28.66, 27.65. m/z calcd. for C₂₆H₃₃N₇O₄=507.3. Found [M+H]⁺=508.5.

### 2-((6-chloropyridin-3-yl)oxy)-N,N-dimethylacetamide (55-i)

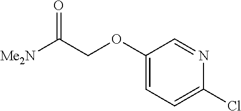 [SMILES: CN(C)C(=O)COc1ccc(Cl)nc1]

Prepared according to general procedure D starting with 6-chloropyridin-3-ol and 2-chloro-N,N-dimethylacetamide to afford the titled compound in 75% yield.

### 55 N,N-dimethyl-2-((6-(((1s,4s)-4-((7-morpholino-1,6-naphthyridin-5-yl)oxy)cyclohexyl)amino)pyridin-3-yl)oxy)acetamide

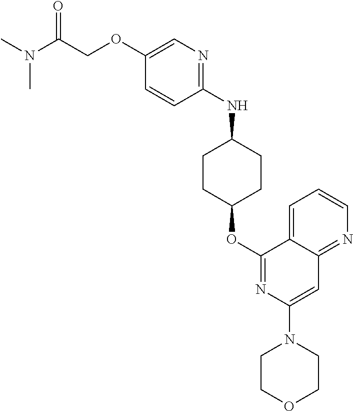 [SMILES: CN(C)C(=O)COc1ccc(N[C@H]2CC[C@@H](Oc3nc(N4CCOCC4)cc4ncccc34)CC2)nc1]

Prepared according to general procedure I starting with 55-i and intermediate 7 to afford the titled compound in 6% yield. ¹H NMR (400 MHz, DMSO) δ 8.86 (dd, J=4.9, 1.7 Hz, 1H), 8.53 (d, J=8.2 Hz, 1H), 8.14 (bs, 1H), 7.70 (d, J=9.7 Hz, 1H), 7.52 (d, J=2.9 Hz, 1H), 7.32 (dd, J=8.1, 4.9 Hz, 1H), 6.98 (d, J=9.3 Hz, 1H), 6.52 (s, 1H), 5.46 (s, 1H), 4.87 (s, 2H), 3.79-3.72 (m, 4H), 3.62-3.55 (m, 4H), 2.97 (s, 3H), 2.85 (s, 3H), 2.17-2.07 (m, 2H), 1.92-1.66 (m, 6H). m/z calcd. for C₂₇H₃₄N₆O₄=506.3. Found [M+H]⁺=507.3.

### 2-((2-chloropyrimidin-4-yl)oxy)-N,N-dimethylacetamide (56-i)

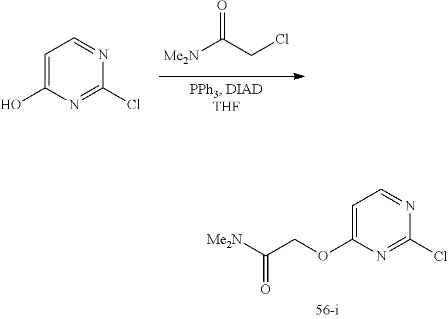 [SMILES: Oc1ccnc(Cl)n1 ; CN(C)C(=O)CCl ; CN(C)C(=O)COc1ccnc(Cl)n1]

To a mixture of commercially available 2-chloropyrimidin-4-ol (1.0 eq), commercially available 2-chloro-N,N-dimethylacetamide (1.1 eq), and triphenylphosphine (1.2 eq) in THF (0.5M) was added DIAD (1.2 eq) dropwise at 5° C. The reaction was stirred at 5° C. for 30 min or until completion of the reaction. The reaction was quenched with sat. NaHCO₃ aq. and extracted with EtOAc. The combined organic layers was dried over Na₂SO₄ and concentrated. Purification via automated flash chromatography (C-18, 10% to 60% ACN in water with 0.1% TFA) gave pure 24 (yield 40%).

### 56 N,N-dimethyl-2-((2-(((1s,4s)-4-((7-morpholino-1,6-naphthyridin-5-yl)oxy)cyclohexyl)amino)pyrimidin-4-yl)oxy)acetamide

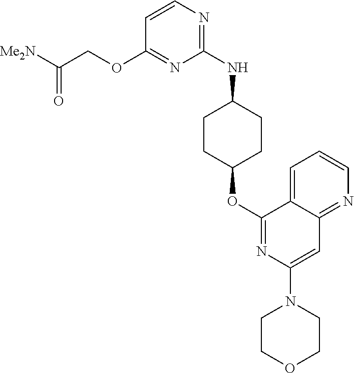 [SMILES: CN(C)C(=O)COc1ccnc(N[C@H]2CC[C@@H](Oc3nc(N4CCOCC4)cc4ncccc34)CC2)n1]

Prepared according to general procedure H starting with 56-i and intermediate 7 to afford the titled compound in 36% yield using sodium carbonate instead of an organic base.

¹H NMR (400 MHz, DMSO) δ 8.88 (dd, J=5.3, 1.7 Hz, 1H), 8.70 (d, J=8.0 Hz, 1H), 8.60 (s, 1H), 8.18 (s, 1H), 7.38 (dd, J=8.1, 5.2 Hz, 1H), 6.51 (s, 1H), 6.49-6.32 (m, 1H), 5.47 (s, 1H), 5.18 (s, 2H), 3.79-3.72 (m, 4H), 3.67-3.60 (m, 4H), 3.14-2.91 (m, 4H), 2.84 (s, 3H), 2.10 (d, J=10.2 Hz, 2H), 1.80 (d, J=9.0 Hz, 6H). m/z calcd. for C₂₆H₃₃N₇O₄=507.3. Found [M+H]⁺=508.2.

### 2-((6-chloropyridazin-3-yl)oxy)-N,N-dimethylacetamide (57-i)

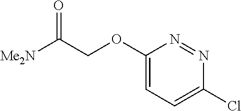 [SMILES: CN(C)C(=O)COc1ccc(Cl)nn1]

Compound 57-i was synthesized with commercially available 6-chloropyridazin-3-ol according to the procedure to prepare compound 56-i (yield 40%).

### 57 N,N-dimethyl-2-((6-(((1s,4s)-4-((7-morpholino-1,6-naphthyridin-5-yl)oxy)cyclohexyl)amino)pyridazin-3-yl)oxy)acetamide

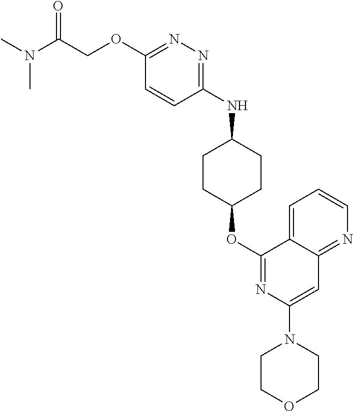 [SMILES: CN(C)C(=O)COc1ccc(N[C@H]2CC[C@@H](Oc3nc(N4CCOCC4)cc4ncccc34)CC2)nn1]

Prepared according to general procedure H starting with 57-i and intermediate 7 to afford the titled compound in 4% yield using potassium carbonate and ACN instead. ¹H NMR (400 MHz, DMSO) δ 8.81 (d, J=4.3 Hz, 1H), 8.44 (d, J=7.6 Hz, 2H), 7.57 (d, J=9.5 Hz, 1H), 7.34 (dd, J=8.2, 4.3 Hz, 1H), 7.01 (d, J=9.5 Hz, 1H), 6.34 (s, 1H), 5.43 (s, 1H), 4.97 (s, 2H), 3.80-3.71 (m, OH), 3.61-3.54 (m, 4H), 3.32-3.25 (m, 4H), 3.04 (s, 3H), 2.85 (s, 3H), 2.14-2.09 (m, 2H), 1.82-1.71 (m, 6H). ¹³C NMR (101 MHz, DMSO) δ 166.32, 159.54, 158.22, 155.71, 155.11, 154.28, 144.35, 138.33, 132.94, 128.21, 119.21, 109.30, 103.72, 70.27, 66.56, 53.25, 49.05, 36.25, 35.56, 27.89, 27.27. m/z calcd. for C₂₆H₃₃N₇O₄=507.3. Found [M+H]⁺=508.2.

### 2-((2-Chloropyrimidin-5-yl)oxy)-N-methoxy-N-methylacetamide (58-i)

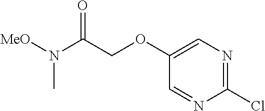

Prepared according to general procedure D starting with 2-chloropyrimidin-5-ol and 2-chloro-N-methoxy-N-methylacetamide to afford the titled compound in 75% yield. ¹H NMR (400 MHz, DMSO-d₆) δ 8.54 (s, 2H), 5.16 (s, 2H), 3.76 (s, 3H), 3.13 (s, 3H). m/z calcd. for C₈H₁₀ClN₃O₃=231.0. Found [M+H]⁺=232.1.

### N-Methoxy-N-methyl-2-((2-(((1s,4s)-4-((7-morpholino-1,6-naphthyridin-5-yl)oxy)cyclohexyl)amino)pyrimidin-5-yl)oxy)acetamide (58)

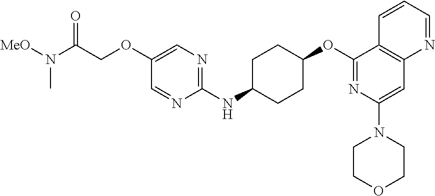 [SMILES: CON(C)C(=O)COc1cnc(N[C@H]2CC[C@@H](Oc3nc(N4CCOCC4)cc4ncccc34)CC2)nc1]

Prepared according to general procedure H starting with 58-i and intermediate 7 to afford the titled compound in 11% yield. ¹H NMR (400 MHz, DMSO-d₆) δ 8.88 (dd, J=5.3, 1.7 Hz, 1H), 8.73 (d, J=8.0 Hz, 1H), 8.12 (s, 2H), 7.39 (dd, J=8.0, 5.3 Hz, 1H), 6.76 (s, 1H), 6.47 (s, 1H), 5.45 (s, 1H), 4.88 (s, 2H), 3.79 (s, 1H), 3.75 (dd, J=5.8, 4.0 Hz, 4H), 3.72 (s, 3H), 3.63 (t, J=4.9 Hz, 4H), 3.12 (s, 3H), 2.20-2.02 (m, 2H), 1.84-1.74 (m, 6H). m/z calcd. for C₂₆H₃₃N₇O₅=523.3. Found [M+H]⁺=524.5.

### tert-Butyl 2-((2-chloropyrimidin-5-yl)oxy)acetate (59-i)

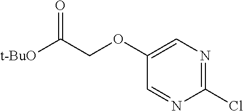 [SMILES: CC(C)(C)OC(=O)COc1cnc(Cl)nc1]

Prepared according to general procedure D starting with 2-chloropyrimidin-5-ol and tert-butyl 2-bromoacetate to afford the titled compound in 94% yield. ¹H NMR (400 MHz, DMSO-d₆) δ 8.55 (s, 2H), 4.90 (s, 2H), 1.44 (s, 9H). m/z calcd. for C₁₀H₁₃ClN₂O₃=244.1. Found [M+H]⁺=245.1.

### 2-((2-(((1s,4s)-4-((7-Morpholino-1,6-naphthyridin-5-yl)oxy)cyclohexyl)amino)pyrimidin-5-yl)oxy)acetic acid (59)

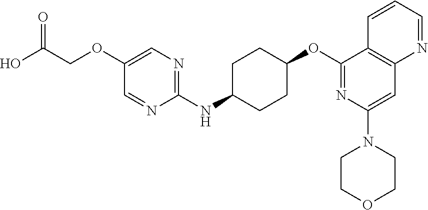 [SMILES: O=C(O)COc1cnc(N[C@H]2CC[C@@H](Oc3nc(N4CCOCC4)cc4ncccc34)CC2)nc1]

Prepared according to general procedures K and P starting with 59-i and intermediate 7 to afford the titled compound in 28% yield over two synthetic steps. ¹H NMR (400 MHz, DMSO-d₆) δ 8.88 (dd, J=5.5, 1.7 Hz, 1H), 8.79-8.70 (m, 1H), 8.12 (s, 2H), 7.41 (dd, J=8.0, 5.4 Hz, 1H), 6.79 (s, 1H), 6.47 (s, 1H), 5.46 (s, 1H), 4.95 (d, J=4.4 Hz, 1H), 4.65 (s, 2H), 3.81 (s, 1H), 3.75 (t, J=4.9 Hz, 4H), 3.64 (t, J=4.8 Hz, 4H), 2.16-2.04 (m, 2H), 1.84-1.72 (m, 6H). m/z calcd. for C₂₄H₂₈N₆O₅=480.2. Found [M+H]⁺=481.2.

### 2-((2-Chloropyrimidin-5-yl)oxy)acetamide (60-i)

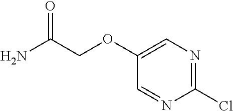 [SMILES: NC(=O)COc1cnc(Cl)nc1]

Prepared according to general procedure D starting with 2-chloropyrimidin-5-ol and 2-iodoacetamide to afford the titled compound in 93% yield. ¹H NMR (400 MHz, DMSO-d₆) δ 8.52 (s, 2H), 7.64 (s, 1H), 7.47 (s, 1H), 4.67 (s, 2H). m/z calcd. for C₆H6ClN₃O₂=187.0. Found [M+H]⁺=187.9.

### 2-((2-(((1s,4s)-4-((7-Morpholino-1,6-naphthyridin-5-yl)oxy)cyclohexyl)amino)pyrimidin-5-yl)oxy)acetamide (60)

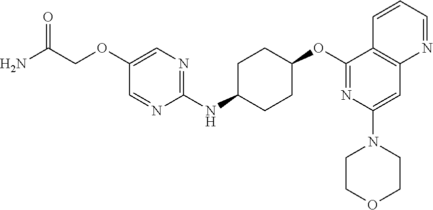 [SMILES: NC(=O)COc1cnc(N[C@H]2CC[C@@H](Oc3nc(N4CCOCC4)cc4ncccc34)CC2)nc1]

Prepared according to general procedure H starting with 60-i and intermediate 7 to afford the titled compound in 26% yield. ¹H NMR (400 MHz, DMSO-d₆) δ 8.88 (dd, J=5.4, 1.7 Hz, 1H), 8.73 (d, J=8.0 Hz, 1H), 8.13 (s, 2H), 7.53 (s, 1H), 7.40 (dd, J=8.0, 5.3 Hz, 2H), 6.79 (s, 1H), 6.48 (s, 1H), 5.45 (s, 1H), 4.40 (s, 2H), 3.81 (s, 1H), 3.75 (t, J=4.8 Hz, 4H), 3.63 (t, J=4.9 Hz, 4H), 2.13-2.06 (m, 2H), 1.84-1.72 (m, 6H). m/z calcd. for C₂₄H₂₉N₇O₄=479.2. Found [M+H]⁺=480.4.

### 2-((2-Chloropyrimidin-5-yl)oxy)-1-morpholinoethan-1-one (61-i)

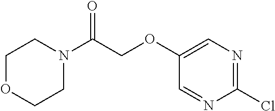 [SMILES: O=C(COc1cnc(Cl)nc1)N1CCOCC1]

Prepared according to general procedure D starting with 2-chloropyrimidin-5-ol and 2-chloro-1-morpholinoethan-1-one to afford the titled compound in 83% yield. ¹H NMR (400 MHz, DMSO-d₆) δ 8.52 (s, 2H), 5.11 (s, 2H), 3.61 (dt, J=19.3, 4.9 Hz, 4H), 3.42 (dt, J=10.1, 4.5 Hz, 4H). m/z calcd. for C₁₀H₁₂ClN₃O₃=257.1. Found [M+H]⁺=258.2.

### 1-Morpholino-2-((2-(((1s,4s)-4-((7-morpholino-1,6-naphthyridin-5-yl)oxy)cyclohexyl)amino)pyrimidin-5-yl)oxy)ethan-1-one (61)

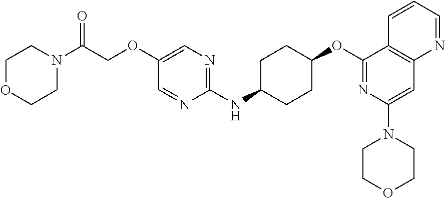 [SMILES: O=C(COc1cnc(N[C@H]2CC[C@@H](Oc3nc(N4CCOCC4)cc4ncccc34)CC2)nc1)N1CCOCC1]

Prepared according to general procedure H starting with 61-i and intermediate 7 to afford the titled compound in 35% yield. ¹H NMR (400 MHz, DMSO-d₆) δ 8.80 (dd, J=4.3, 1.8 Hz, 1H), 8.37 (dd, J=8.1, 1.9 Hz, 1H), 8.10 (s, 2H), 7.22 (dd, J=8.2, 4.3 Hz, 1H), 6.77 (d, J=8.0 Hz, 1H), 6.53 (s, 1H), 5.41 (s, 1H), 4.80 (s, 2H), 3.79 (s, 1H), 3.75 (t, J=4.9 Hz, 4H), 3.59 (dt, J=11.2, 4.4 Hz, 4H), 3.50 (t, J=4.9 Hz, 4H), 3.43 (d, J=5.4 Hz, 4H), 2.14-2.02 (m, 2H), 1.82-1.71 (m, 6H). ¹³C NMR (100 MHz, DMSO-d₆) δ 166.51, 158.89, 158.26, 156.95, 155.73, 155.32, 147.77, 146.80, 145.33, 132.73, 118.30, 109.09, 92.42, 70.60, 67.92, 66.34, 49.07, 45.85, 45.06, 28.67, 27.65. m/z calcd. for C₂₈H₃₅N₅O₃=549.3. Found [M+H]⁺=550.4.

### 1-(4-Methylpiperazin-1-yl)-2-((2-(((1s,4s)-4-((7-morpholino-1,6-naphthyridin-5-yl)oxy)cyclohexyl)amino)pyrimidin-5-yl)oxy)ethan-1-one (62)

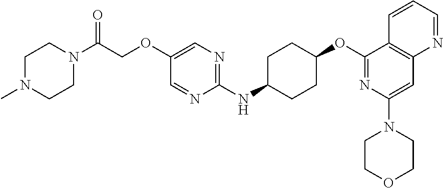 [SMILES: CN1CCN(C(=O)COc2cnc(N[C@H]3CC[C@@H](Oc4nc(N5CCOCC5)cc5ncccc45)CC3)nc2)CC1]

Prepared according to general procedure 0 starting with acid 59 and 1-methylpiperazine to afford the titled compound in 58% yield. ¹H NMR (400 MHz, DMSO-d₆) δ 9.86 (s, 1H), 8.87 (dd, J=5.2, 1.7 Hz, 1H), 8.69 (d, J=8.1 Hz, 1H), 8.12 (s, 2H), 7.38 (dd, J=8.1, 5.2 Hz, 1H), 6.77 (s, 1H), 6.49 (s, 1H), 5.45 (s, 1H), 4.86 (d, J=12.0 Hz, 2H), 4.40 (s, 1H), 3.94 (s, 2H), 3.75 (dd, J=5.8, 3.9 Hz, 5H), 3.62 (t, J=4.9 Hz, 4H), 3.50-3.26 (m, 2H), 3.18-2.91 (m, 2H), 2.83 (s, 3H), 2.17-2.04 (m, 2H), 1.81-1.74 (m, 6H). m/z calcd. for C₂₉H₃₈N₈O₄=562.3. Found [M+H]⁺=563.3.

### 2-((2-(((1s,4s)-4-((7-Morpholino-1,6-naphthyridin-5-yl)oxy)cyclohexyl)amino)pyrimidin-5-yl)oxy)-1-(piperazin-1-yl)ethan-1-one (63)

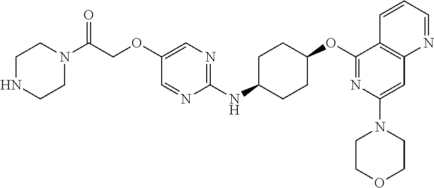 [SMILES: O=C(COc1cnc(N[C@H]2CC[C@@H](Oc3nc(N4CCOCC4)cc4ncccc34)CC2)nc1)N1CCNCC1]

Prepared according to general procedures 0 and P starting with acid 59 and tert-butyl piperazine-1-carboxylate to afford the titled compound in 65% yield over two synthetic steps. ¹H NMR (400 MHz, DMSO-d₆) δ 8.80 (dd, J=4.3, 1.9 Hz, 1H), 8.37 (dd, J=8.2, 1.8 Hz, 1H), 8.27 (s, 1H), 8.09 (s, 2H), 7.22 (dd, J=8.2, 4.3 Hz, 1H), 6.80 (d, J=8.0 Hz, 1H), 6.53 (s, 1H), 5.41 (s, 1H), 4.78 (s, 2H), 3.78 (s, 1H), 3.74 (t, J=4.8 Hz, 4H), 3.50 (t, J=4.8 Hz, 4H), 3.44-3.29 (m, 4H), 2.82-2.62 (m, 4H), 2.11-2.04 (m, 2H), 1.82-1.71 (m, 6H). m/z calcd. for C₂₈H₃₆N₈O₄=548.3. Found [M+H]⁺=549.3.

### 2-((2-Chloropyrimidin-5-yl)oxy)-N,N-dimethylethan-1-amine (64-i)

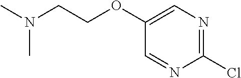

Prepared according to general procedure E starting with 2-chloropyrimidin-5-ol and 2-(dimethylamino)ethan-1-ol to afford the titled compound in 62% yield. ¹H NMR (400 MHz, DMSO-d₆) δ 8.55 (s, 2H), 4.23 (t, J=5.6 Hz, 2H), 2.64 (t, J=5.6 Hz, 2H), 2.21 (s, 6H). m/z calcd. for C₈H₁₂ClN₃O=201.1. Found [M+H]⁺=202.2.

### 5-(2-(Dimethylamino)ethoxy)-N-((1s,4s)-4-((7-morpholino-1,6-naphthyridin-5-yl)oxy)cyclohexyl)pyrimidin-2-amine (64)

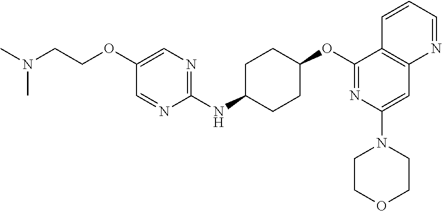 [SMILES: CN(C)CCOc1cnc(N[C@H]2CC[C@@H](Oc3nc(N4CCOCC4)cc4ncccc34)CC2)nc1]

Prepared according to general procedure K starting with 64-i and intermediate 7 to afford the titled compound in 50% yield. ¹H NMR (400 MHz, DMSO-d₆) δ 8.80 (dd, J=4.4, 1.8 Hz, 1H), 8.37 (dd, J=8.2, 1.8 Hz, 1H), 8.11 (s, 2H), 7.22 (dd, J=8.2, 4.3 Hz, 1H), 6.72 (d, J=8.0 Hz, 1H), 6.53 (s, 1H), 5.41 (s, 1H), 4.01 (t, J=5.8 Hz, 2H), 3.82-3.71 (m, 5H), 3.50 (t, J=4.8 Hz, 4H), 2.56 (t, J=5.8 Hz, 2H), 2.20 (s, 6H), 2.12-2.06 (m, 2H), 1.87-1.67 (m, 6H). ¹³C NMR (100 MHz, DMSO-d₆) δ 158.89, 158.12, 156.95, 155.72, 155.32, 146.54, 145.59, 132.73, 118.30, 109.09, 92.42, 70.60, 68.28, 66.34, 58.29, 49.06, 45.99, 45.84, 28.68, 27.67. m z calcd. for C₂₆H₃₅N₇O₃=493.3. Found [M+H]⁺=494.3.

### tert-Butyl (2-((2-chloropyrimidin-5-yl)oxy)ethyl)carbamate (65-i)

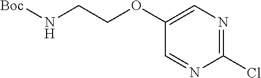 [SMILES: CC(C)(C)OC(=O)NCCOc1cnc(Cl)nc1]

Prepared according to general procedure D starting with 2-chloropyrimidin-5-ol and tert-butyl (2-chloroethyl)carbamate to afford the titled compound in 84% yield. ¹H NMR (400 MHz, DMSO-d₆) δ 8.53 (s, 2H), 7.05 (d, J=6.8 Hz, 1H), 4.16 (t, J=5.6 Hz, 2H), 3.31 (t, J=5.7 Hz, 2H), 1.37 (s, 9H). m/z calcd. for C₁₁H₁₆ClN₃O₃=273.1. Found [M+H]⁺=274.2.

### 5-(2-Aminoethoxy)-N-((1s,4s)-4-((7-morpholino-1,6-naphthyridin-5-yl)oxy)cyclohexyl)pyrimidin-2-amine (65)

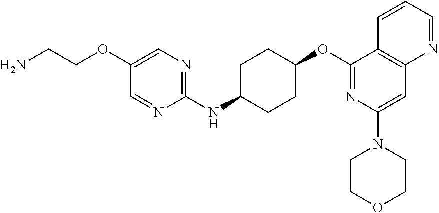 [SMILES: NCCOc1cnc(N[C@H]2CC[C@@H](Oc3nc(N4CCOCC4)cc4ncccc34)CC2)nc1]

Prepared according to general procedures K and P starting with 65-i and intermediate 7 to afford the titled compound in 65% yield over two synthetic steps. ¹H NMR (400 MHz, DMSO-d₆) δ 8.86 (dd, J=5.0, 1.7 Hz, 1H), 8.63 (d, J=8.0 Hz, 1H), 8.18 (s, 2H), 7.95 (s, 3H), 7.35 (dd, J=8.1, 5.0 Hz, 1H), 6.84 (d, J=7.6 Hz, 1H), 6.50 (s, 1H), 5.45 (s, 1H), 4.13 (t, J=5.1 Hz, 2H), 3.81 (s, 1H), 3.75 (dd, J=5.8, 3.9 Hz, 4H), 3.60 (d, J=4.9 Hz, 4H), 3.18 (dt, J=10.9, 5.6 Hz, 2H), 2.09 (d, J=7.2 Hz, 2H), 1.78 (d, J=7.2 Hz, 6H). m/z calcd. for C₂₄H₃₁N₇O₃=465.3. Found [M+H]⁺=466.2.

### N-(2-((2-chloropyrimidin-5-yl)oxy)ethyl)acetamide (66-i)

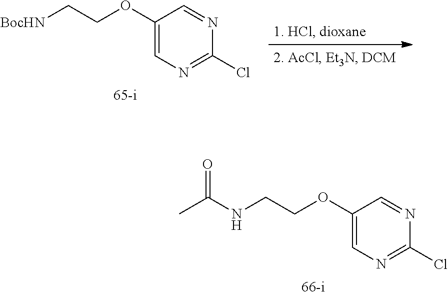 [SMILES: CC(C)(C)OC(=O)NCCOc1cnc(Cl)nc1 ; CC(=O)NCCOc1cnc(Cl)nc1]

65-i (0.96 mmol) was dissolved in 4 M HCl in dioxane and stirred at room temperature for 1 h. The mixture was evaporated and dried under vacuum to afford the amine hydrochloride as a white solid. This solid was suspended in DCM (0.1 M) then treated with Et₃N (5.0 eq) and AcCl (1.2 eq) and stirred at room temperature. After 18 h, the reaction mixture was evaporated and the crude product was purified by flash chromatography (MeOH/DCM) to give 66-i (162 mg, 78%) as a white solid. ¹H NMR (400 MHz, CDCl₃) δ 8.32 (s, 2H), 5.89 (bs, 1H), 4.17 (t, J=5.2 Hz, 2H), 3.73 (dd, J=5.4, 5.4 Hz, 2H), 2.05 (s, 3H).

### N-(2-(4-(((1S,4S)-4-((7-morpholino-1,6-naphthyridin-5-yl)oxy)cyclohexyl)amino)phenoxy)ethyl)acetamide/66

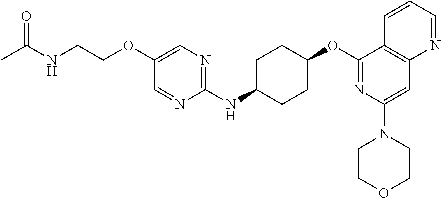 [SMILES: CC(=O)NCCOc1cnc(N[C@H]2CC[C@@H](Oc3nc(N4CCOCC4)cc4ncccc34)CC2)nc1]

Prepared using general procedure H from 66-i (2.0 eq) and intermediate 7 (1.0 eq). 180° C. for 5 h. Purified by prep HPLC (ACN/H₂O+TFA). ¹H NMR (400 MHz, DMSO-d₆) δ 8.88 (dd, J=5.3, 1.7 Hz, 1H), 8.73 (d, J=8.0 Hz, 1H), 8.13 (s, 2H), 8.09-8.05 (m, 1H), 7.40 (dd, J=8.0, 5.3 Hz, 1H), 6.74 (bs, 1H), 6.47 (s, 1H), 5.46 (bs, 1H), 3.95 (t, J=5.6 Hz, 2H), 3.84-3.78 (m, 1H), 3.78-3.72 (m, 4H), 3.66-3.61 (m, 4H), 3.36 (t, J=5.6 Hz, 2H), 2.14-2.06 (m, 2H), 1.83 (s, 3H), 1.81-1.73 (m, 6H).

### N-(2-((2-chloropyrimidin-5-yl)oxy)ethyl)methanesulfonamide (67-i)

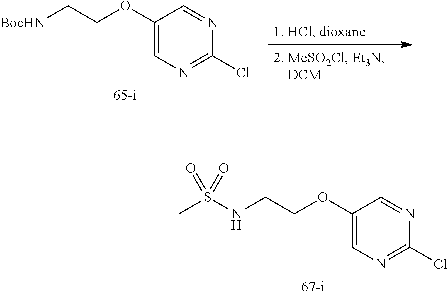 [SMILES: CC(C)(C)OC(=O)NCCOc1cnc(Cl)nc1 ; CS(=O)(=O)NCCOc1cnc(Cl)nc1]

65-i (0.89 mmol) was dissolved in 4 M HCl in dioxane and stirred at room temperature for 1 h. The mixture was evaporated and dried under vacuum to afford the amine hydrochloride as a white solid. This solid was suspended in DCM (0.1 M) then treated with Et₃N (5.0 eq) and MeSO₂Cl (1.2 eq) and stirred at room temperature. After 18 h, the reaction mixture was evaporated and the crude product was purified by flash chromatography (MeOH/DCM) to give 67-i (198 mg, 88%) as a white solid. ¹H NMR (400 MHz, CDCl₃) δ 8.34 (s, 2H), 4.82 (bs, 1H), 4.24 (t, J=5.1 Hz, 2H), 3.62 (dt, J=6.4, 5.1 Hz, 2H), 3.06 (s, 3H).

### N-(2-(4-(((1S,4S)-4-((7-morpholino-1,6-naphthyridin-5-yl)oxy)cyclohexyl)amino)phenoxy)ethyl)methanesulfonamide/67

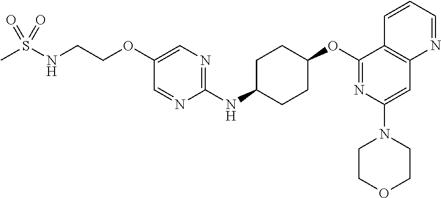 [SMILES: CS(=O)(=O)NCCOc1cnc(N[C@H]2CC[C@@H](Oc3nc(N4CCOCC4)cc4ncccc34)CC2)nc1]

Prepared using general procedure H from 67-i (2.0 eq) and intermediate 7 (1.0 eq). 180° C. for 4 h. Purified by prep HPLC (ACN/H₂O+TFA). ¹H NMR (400 MHz, DMSO-d₆) δ 8.89 (dd, J=5.5, 1.7 Hz, 1H), 8.78 (d, J=7.9 Hz, 1H), 8.15 (s, 2H), 7.42 (dd, J=8.0, 5.4 Hz, 1H), 7.29 (t, J=5.9 Hz, 1H), 6.81 (bs, 1H), 6.47 (s, 1H), 5.46 (bs, 1H), 4.00 (t, J=5.5 Hz, 2H), 3.84-3.79 (m, 1H), 3.77-3.72 (m, 4H), 3.67-3.62 (m, 4H), 3.31-3.27 (m, 2H), 2.95 (s, 3H), 2.12-2.04 (m, 2H), 1.83-1.71 (m, 6H).

### 4-(2-((2-Chloropyrimidin-5-yl)oxy)ethyl)morpholine (68-i)

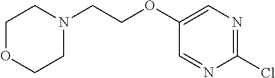 [SMILES: Clc1ncc(OCCN2CCOCC2)cn1]

Prepared according to general procedure E starting with 2-chloropyrimidin-5-ol and 2-morpholinoethan-1-ol to afford the titled compound in 97% yield. ¹H NMR (400 MHz, DMSO-d₆) δ 8.56 (s, 2H), 4.27 (t, J=5.6 Hz, 2H), 3.60-3.53 (m, 4H), 2.71 (t, J=5.6 Hz, 2H), 2.46 (t, J=4.7 Hz, 4H). m/z calcd. for C₁₀H₁₄ClN₃O₂=243.1. Found [M+H]⁺=244.1.

### N-((1s,4s)-4-((7-Morpholino-1,6-naphthyridin-5-yl)oxy)cyclohexyl)-5-(2-morpholinoethoxy)pyrimidin-2-amine (68)

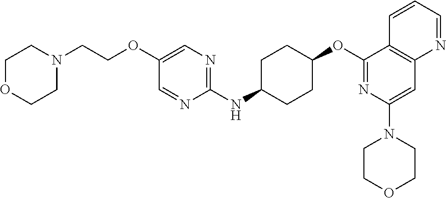 [SMILES: c1cnc2cc(N3CCOCC3)nc(O[C@H]3CC[C@@H](Nc4ncc(OCCN5CCOCC5)cn4)CC3)c2c1]

Prepared according to general procedure K starting with 68-i and intermediate 7 to afford the titled compound in 46% yield. ¹H NMR (400 MHz, DMSO-d₆) δ 10.02 (s, 1H), 8.87 (dd, J=5.2, 1.7 Hz, 1H), 8.66 (d, J=8.0 Hz, 1H), 8.21 (s, 2H), 7.37 (dd, J=8.1, 5.2 Hz, 1H), 6.86 (s, 1H), 6.50 (s, 1H), 5.45 (s, 1H), 4.32 (t, J=4.9 Hz, 2H), 3.99 (s, 2H), 3.82 (s, 1H), 3.75 (t, J=4.9 Hz, 4H), 3.61 (t, J=4.9 Hz, 4H), 3.58-3.51 (m, 4H), 3.31-3.06 (m, 2H), 2.12-2.06 (m, 2H), 1.81-1.77 (m, 6H). m/z calcd. for C₂₈H₃₇N₇O₄=535.3. Found [M+H]⁺=536.3.

### 2-Chloro-5-(2-(4-methylpiperazin-1-yl)ethoxy)pyrimidine (69-i)

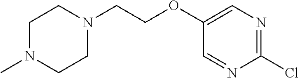 [SMILES: CN1CCN(CCOc2cnc(Cl)nc2)CC1]

Prepared according to general procedure E starting with 2-chloropyrimidin-5-ol and 2-(4-methylpiperazin-1-yl)ethan-1-ol to afford the titled compound in 99% yield. ¹H NMR (400 MHz, DMSO-d₆) δ 8.55 (s, 2H), 4.25 (t, J=5.7 Hz, 2H), 2.70 (t, J=5.8 Hz, 2H), 2.50-2.24 (m, 8H), 2.17 (s, 3H). m/z calcd. for C₁₁H₁₇ClN₄O=256.1. Found [M+H]⁺=257.2.

### 5-(2-(4-Methylpiperazin-1-yl)ethoxy)-N-((1s,4s)-4-((7-morpholino-1,6-naphthyridin-5-yl)oxy)cyclohexyl)pyrimidin-2-amine (69)

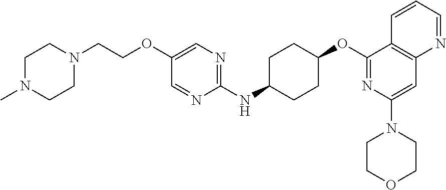 [SMILES: CN1CCN(CCOc2cnc(N[C@H]3CC[C@@H](Oc4nc(N5CCOCC5)cc5ncccc45)CC3)nc2)CC1]

Prepared according to general procedure K starting with 69-i and intermediate 7 to afford the titled compound in 31% yield. ¹H NMR (400 MHz, DMSO-d₆) δ 8.88 (dd, J=5.2, 1.7 Hz, 1H), 8.70 (d, J=7.9 Hz, 1H), 8.15 (s, 2H), 7.39 (dd, J=8.0, 5.2 Hz, 1H), 6.77 (s, 1H), 6.49 (s, 1H), 5.45 (s, 1H), 4.12 (s, 2H), 3.81 (s, 1H), 3.75 (dd, J=5.8, 4.0 Hz, 4H), 3.63 (d, J=4.9 Hz, 4H), 3.53-3.31 (m, 3H), 3.32-3.02 (m, 4H), 2.99-2.94 (m, 3H), 2.79 (s, 3H), 2.12-2.07 (m, 2H), 1.81-1.74 (m, 6H). m/z calcd. for C₂₉H₄₀N₈O₃=548.3. Found [M+H]⁺=549.3.

### tert-Butyl 4-(2-((2-chloropyrimidin-5-yl)oxy)ethyl)piperazine-1-carboxylate (70-i)

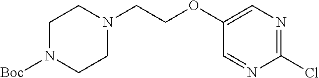 [SMILES: CC(C)(C)OC(=O)N1CCN(CCOc2cnc(Cl)nc2)CC1]

Prepared according to general procedure E starting with 2-chloropyrimidin-5-ol and tert-butyl 4-(2-hydroxyethyl)piperazine-1-carboxylate to afford the titled compound in 83% yield. ¹H NMR (400 MHz, DMSO-d₆) δ 8.56 (d, J=2.0 Hz, 2H), 4.26 (d, J=5.7 Hz, 2H), 2.80-2.71 (m, 2H), 2.55-2.39 (m, 8H), 1.40 (d, J=2.0 Hz, 9H). m/z calcd. for C₁₅H₂₃ClN₄O₃=342.2. Found [M+H]⁺=343.2.

### N-((1s,4s)-4-((7-Morpholino-1,6-naphthyridin-5-yl)oxy)cyclohexyl)-5-(2-(piperazin-1-yl)ethoxy)pyrimidin-2-amine (70)

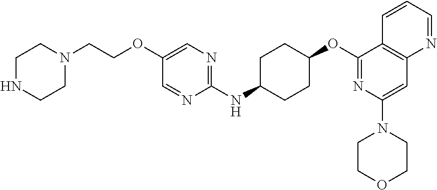 [SMILES: c1cnc2cc(N3CCOCC3)nc(O[C@H]3CC[C@@H](Nc4ncc(OCCN5CCNCC5)cn4)CC3)c2c1]

Prepared according to general procedures K and P starting with 70-i and intermediate 7 to afford the titled compound in 64% yield over two synthetic steps. ¹H NMR (400 MHz, DMSO-d₆) δ 8.94 (s, 2H), 8.89 (dd, J=5.3, 1.7 Hz, 1H), 8.71 (d, J=8.0 Hz, 1H), 8.17 (s, 2H), 7.39 (dd, J=8.0, 5.3 Hz, 1H), 6.85 (s, 1H), 6.51 (s, 1H), 5.45 (s, 1H), 4.20 (t, J=5.0 Hz, 2H), 3.81 (s, 1H), 3.75 (dd, J=5.8, 3.9 Hz, 4H), 3.63 (t, J=4.8 Hz, 4H), 3.43-2.97 (m, 10H), 2.13-2.06 (m, 2H), 1.83-1.72 (m, 6H). m/z calcd. for C₂₈H₃₈N₈O₃=534.3. Found [M+H]⁺=535.3.

### N-Methyl-2-((2-(((1s,4s)-4-((7-morpholino-1,6-naphthyridin-5-yl)oxy)cyclohexyl)amino)pyrimidin-5-yl)oxy)acetamide (71)

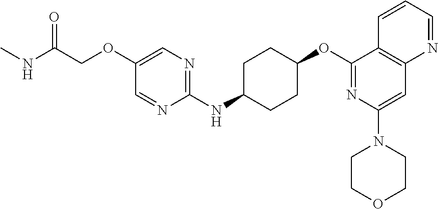 [SMILES: CNC(=O)COc1cnc(N[C@H]2CC[C@@H](Oc3nc(N4CCOCC4)cc4ncccc34)CC2)nc1]

Prepared according to general procedure 0 starting with acid 59 and methylamine (2.0M THF) to afford the titled compound in 54% yield. ¹H NMR (400 MHz, DMSO-d₆) δ 8.89 (dd, J=5.4, 1.7 Hz, 1H), 8.75 (d, J=8.0 Hz, 1H), 8.14 (s, 2H), 8.06 (d, J=4.9 Hz, 1H), 7.41 (dd, J=8.0, 5.4 Hz, 1H), 6.84 (s, 1H), 6.47 (s, 1H), 5.45 (s, 1H), 4.44 (s, 2H), 3.81 (s, 1H), 3.75 (dd, J=5.8, 3.9 Hz, 4H), 3.64 (t, J=4.8 Hz, 4H), 2.65 (d, J=4.7 Hz, 3H), 2.12-2.06 (m, 2H), 1.83-1.74 (m, 6H). m/z calcd. for C₂₅H₃₁N₇O₄=493.2. Found [M+H]⁺=494.3.

### N-Methoxy-2-((2-(((1s,4s)-4-((7-morpholino-1,6-naphthyridin-5-yl)oxy)cyclohexyl)amino)pyrimidin-5-yl)oxy)acetamide (72)

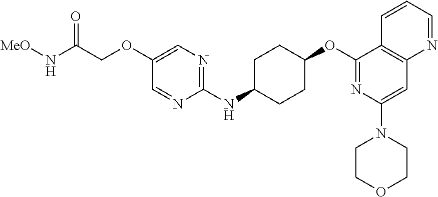 [SMILES: CONC(=O)COc1cnc(N[C@H]2CC[C@@H](Oc3nc(N4CCOCC4)cc4ncccc34)CC2)nc1]

Prepared according to general procedure 0 starting with acid 59 and methoxyamine hydrochloride to afford the titled compound in 43% yield. ¹H NMR (400 MHz, DMSO-d₆) δ 11.46 (s, 1H), 8.89 (dd, J=5.4, 1.6 Hz, 1H), 8.77 (d, J=7.9 Hz, 1H), 8.14 (s, 2H), 7.42 (dd, J=8.0, 5.5 Hz, 1H), 6.85 (s, 1H), 6.46 (s, 1H), 5.46 (s, 1H), 4.46 (s, 2H), 3.81 (s, 1H), 3.75 (t, J=4.8 Hz, 4H), 3.65 (t, J=4.9 Hz, 4H), 3.62 (s, 3H), 2.13-2.06 (m, 2H), 1.83-1.71 (m, 6H). m/z calcd. for C₂₅H₃₁N₇O₅=509.2. Found [M+H]⁺=510.2.

### 2-((2-(((1s,4s)-4-((7-Morpholino-1,6-naphthyridin-5-yl)oxy)cyclohexyl)amino)pyrimidin-5-yl)oxy)-1-thiomorpholinoethan-1-one (73)

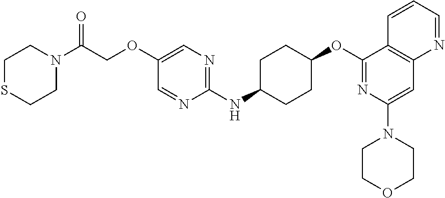 [SMILES: O=C(COc1cnc(N[C@H]2CC[C@@H](Oc3nc(N4CCOCC4)cc4ncccc34)CC2)nc1)N1CCSCC1]

Prepared according to general procedure 0 starting with acid 59 and thiomorpholine to afford the titled compound in 46% yield. ¹H NMR (400 MHz, DMSO-d₆) δ 8.88 (dd, J=5.4, 1.7 Hz, 1H), 8.75 (d, J=8.0 Hz, 1H), 8.11 (s, 2H), 7.41 (dd, J=8.0, 5.4 Hz, 1H), 6.78 (s, 1H), 6.46 (s, 1H), 5.45 (s, 1H), 4.81 (s, 2H), 3.88-3.31 (m, 13H), 2.70-2.64 (m, 2H), 2.60-2.53 (m, 2H), 2.14-2.04 (m, 2H), 1.84-1.72 (m, 6H). m/z calcd. for C₂₈H₃₅N₇O₄S=565.2. Found [M+H]⁺=566.2.

### 2-((2-(((1s,4s)-4-((7-Morpholino-1,6-naphthyridin-5-yl)oxy)cyclohexyl)amino)pyrimidin-5-yl)oxy)-1-(pyrrolidin-1-yl)ethan-1-one (74)

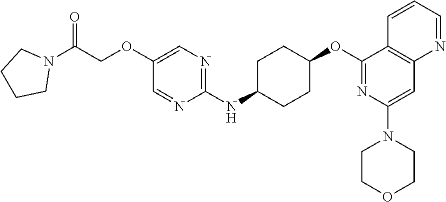 [SMILES: O=C(COc1cnc(N[C@H]2CC[C@@H](Oc3nc(N4CCOCC4)cc4ncccc34)CC2)nc1)N1CCCC1]

Prepared according to general procedure 0 starting with acid 59 and pyrrolidine to afford the titled compound in 34% yield. ¹H NMR (400 MHz, DMSO-d₆) δ 8.88 (dd, J=5.4, 1.6 Hz, 1H), 8.75 (d, J=8.1 Hz, 1H), 8.11 (s, 2H), 7.40 (dd, J=8.0, 5.4 Hz, 1H), 6.77 (s, 1H), 6.46 (s, 1H), 5.45 (s, 1H), 4.70 (s, 2H), 3.95-3.12 (m, 13H), 2.11-2.07 (m, 2H), 1.94-1.83 (m, 2H), 1.82-1.71 (m, 8H). m/z calcd. for C₂₈H₃₅N₇O₄=533.3. Found [M+H]⁺=534.3.

### 1-(Azetidin-1-yl)-2-((2-(((1s,4s)-4-((7-morpholino-1,6-naphthyridin-5-yl)oxy)cyclohexyl)amino)pyrimidin-5-yl)oxy)ethan-1-one (75)

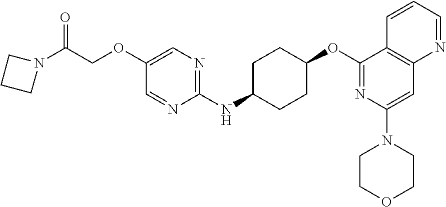 [SMILES: O=C(COc1cnc(N[C@H]2CC[C@@H](Oc3nc(N4CCOCC4)cc4ncccc34)CC2)nc1)N1CCC1]

Prepared according to general procedure 0 starting with acid 59 and azetidine to afford the titled compound in 61% yield. ¹H NMR (400 MHz, DMSO-d₆) δ 8.80 (dd, J=4.4, 1.9 Hz, 1H), 8.38 (d, J=1.9 Hz, 1H), 8.10 (s, 2H), 7.26-7.18 (m, 1H), 6.83 (d, J=8.0 Hz, 1H), 6.53 (s, 1H), 5.41 (s, 1H), 4.54 (s, 2H), 4.20 (t, J=7.7 Hz, 2H), 3.90 (t, J=7.7 Hz, 2H), 3.82-3.71 (m, 5H), 3.50 (t, J=4.9 Hz, 4H), 2.24 (p, J=7.7 Hz, 2H), 2.10-2.06 (m, 2H), 1.79-1.71 (m, 6H). m/z calcd. for C₂₇H₃₃N₇O₄=519.3. Found [M+H]⁺=520.2.

### 1-(3-Fluoroazetidin-1-yl)-2-((2-(((1s,4s)-4-((7-morpholino-1,6-naphthyridin-5-yl)oxy)cyclohexyl)amino)pyrimidin-5-yl)oxy)ethan-1-one (76)

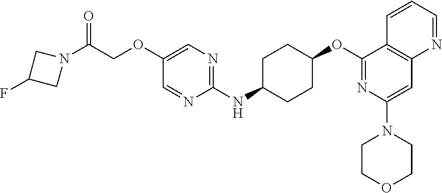 [SMILES: O=C(COc1cnc(N[C@H]2CC[C@@H](Oc3nc(N4CCOCC4)cc4ncccc34)CC2)nc1)N1CC(F)C1]

Prepared according to general procedure 0 starting with acid 59 and 3-fluoroazetidine hydrochloride to afford the titled compound in 50% yield. ¹H NMR (400 MHz, DMSO-d₆) δ 8.88 (dd, J=5.4, 1.8 Hz, 1H), 8.75 (d, J=7.3 Hz, 1H), 8.12 (s, 2H), 7.41 (dd, J=8.0, 5.3 Hz, 1H), 6.81 (s, 1H), 6.46 (d, J=2.1 Hz, 1H), 5.50 (dq, J=6.0, 3.0 Hz, 0.5H), 5.45 (s, 1H), 5.35 (dq, J=6.1, 3.0 Hz, 0.5H), 4.61 (s, 2H), 4.60-4.47 (m, 1H), 4.37-4.18 (m, 2H), 4.02-3.89 (m, 1H), 3.84-3.34 (m, 9H), 2.12-2.06 (m, 2H), 1.83-1.71 (m, 6H). ¹³C NMR (100 MHz, d₆-DMSO) δ 168.26, 159.56, 158.23, 146.56, 145.08, 116.90, 110.80, 84.76, 82.77, 72.09, 67.40, 66.23, 58.37, 58.12, 56.54, 56.29, 48.90, 45.48, 28.43, 27.51.

¹⁹F NMR (377 MHz, DMSO) δ −74.25 (TFA), −179.63.

m/z calcd. for C₂₇H₃₂N₇O₄=537.3. Found [M+H]⁺=538.2.

### 1-(3-Hydroxyazetidin-1-yl)-2-((2-(((1s,4s)-4-((7-morpholino-1,6-naphthyridin-5-yl)oxy)cyclohexyl)amino)pyrimidin-5-yl)oxy)ethan-1-one (77)

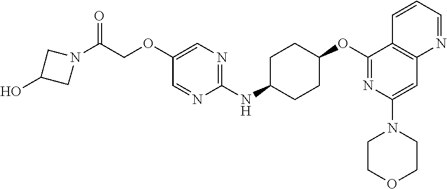 [SMILES: O=C(COc1cnc(N[C@H]2CC[C@@H](Oc3nc(N4CCOCC4)cc4ncccc34)CC2)nc1)N1CC(O)C1]

Prepared according to general procedure 0 starting with acid 59 and 3-hydroxyazetidine hydrochloride to afford the titled compound in 50% yield. ¹H NMR (400 MHz, DMSO-d₆) δ 8.89 (d, J=1.9 Hz, 1H), 8.75 (d, J=7.8 Hz, 1H), 8.10 (s, 2H), 7.41 (dd, J=8.1, 5.3 Hz, 1H), 6.81 (s, 1H), 6.46 (s, 1H), 5.45 (s, 1H), 4.57 (d, J=2.1 Hz, 2H), 4.53-4.44 (m, 1H), 4.38 (dd, J=9.0, 7.1 Hz, 1H), 4.10 (dd, J=10.3, 6.8 Hz, 1H), 3.93 (dd, J=9.5, 4.4 Hz, 1H), 3.87-3.40 (m, 10H), 2.30-2.18 (m, 1H), 2.11-2.06 (m, 2H), 1.83-1.74 (m, 6H). m/z calcd. for C₂₇H₃₃N₇O₅=535.3. Found [M+H]⁺=536.2.

### 2-(((2-Chloropyrimidin-5-yl)oxy)methyl)oxazole (78-i)

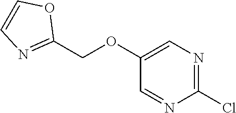 [SMILES: Clc1ncc(OCc2ncco2)cn1]

Prepared according to general procedure D starting with 2-chloropyrimidin-5-ol and 2-(chloromethyl)oxazole to afford the titled compound in 81% yield. ¹H NMR (400 MHz, DMSO-d₆) δ 8.67 (s, 2H), 8.23 (d, J=0.9 Hz, 1H), 7.32 (d, J=0.9 Hz, 1H), 5.47 (s, 2H). m z calcd. for C₈H6ClN₃O₂=211.0. Found [M+H]⁺=212.0.

### N-((1s,4s)-4-((7-Morpholino-1,6-naphthyridin-5-yl)oxy)cyclohexyl)-5-(oxazol-2-ylmethoxy)pyrimidin-2-amine (78)

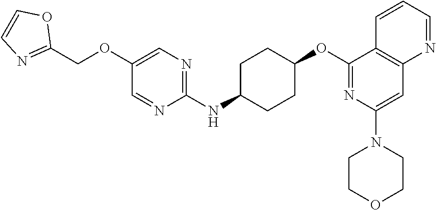 [SMILES: c1cnc2cc(N3CCOCC3)nc(O[C@H]3CC[C@@H](Nc4ncc(OCc5ncco5)cn4)CC3)c2c1]

Prepared according to general procedure H starting with 78-i and intermediate 7 to afford the titled compound in 33% yield. ¹H NMR (400 MHz, DMSO-d₆) δ 8.88 (dd, J=5.4, 1.6 Hz, 1H), 8.72 (d, J=8.0 Hz, 1H), 8.19 (s, 1H), 8.18 (s, 2H), 7.39 (dd, J=8.0, 5.3 Hz, 1H), 7.28 (s, 1H), 6.87 (s, 1H), 6.47 (s, 1H), 5.45 (s, 1H), 5.19 (s, 2H), 3.92-3.49 (m, 9H), 2.12-2.06 (m, 2H), 1.83-1.71 (m, 6H). ¹³C NMR (100 MHz, DMSO-d₆) δ 159.69, 159.52, 158.49, 158.18, 147.49, 144.79, 141.39, 127.86, 116.98, 110.68, 71.98, 66.24, 64.29, 48.89, 45.50, 28.44, 27.50. m/z calcd. for C₂₆H₂₉N₇O₄=503.2. Found [M+H]⁺=504.2.

### 2-Chloro-5-((1-methyl-1H-imidazol-2-yl)methoxy)pyrimidine (79-i)

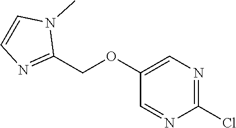

Prepared according to general procedure D starting with 2-chloropyrimidin-5-ol and 2-(chloromethyl)-1-methyl-1H-imidazole hydrochloride to afford the titled compound in 19% yield. ¹H NMR (400 MHz, DMSO-d₆) δ 8.67 (s, 2H), 7.23 (d, J=1.2 Hz, 1H), 6.90 (d, J=1.2 Hz, 1H), 5.36 (s, 2H), 3.70 (s, 3H). m/z calcd. for C₉H9ClN₄0=224.0. Found [M+H]⁺=225.0.

### 5-((1-methyl-1H-imidazol-2-yl)methoxy)-N-((1s,4s)-4-((7-morpholino-1,6-naphthyridin-5-yl)oxy)cyclohexyl)pyrimidin-2-amine (79)

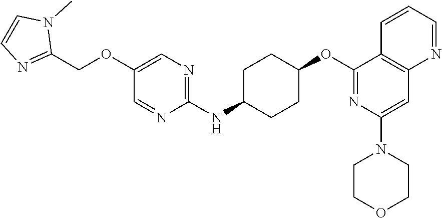 [SMILES: Cn1ccnc1COc1cnc(N[C@H]2CC[C@@H](Oc3nc(N4CCOCC4)cc4ncccc34)CC2)nc1]

Prepared according to general procedure H starting with 79-i and intermediate 7 to afford the titled compound in 12% yield. ¹H NMR (400 MHz, DMSO-d₆) δ 8.88 (dd, J=5.2, 1.7 Hz, 1H), 8.66 (d, J=8.0 Hz, 1H), 8.23 (s, 2H), 7.75 (d, J=1.9 Hz, 1H), 7.67 (d, J=1.9 Hz, 1H), 7.37 (dd, J=8.1, 5.2 Hz, 1H), 7.01 (d, J=7.9 Hz, 1H), 6.50 (s, 1H), 5.45 (s, 1H), 5.38 (s, 2H), 3.88 (s, 3H), 3.86-3.46 (m, 9H), 2.12-2.06 (m, 2H), 1.80-1.74 (m, 6H). ¹³C NMR (100 MHz, DMSO-d₆) δ 159.40, 158.88, 157.98, 154.20, 144.39, 142.25, 124.97, 120.08, 117.18, 110.42, 71.72, 66.25, 62.07, 48.95, 45.55, 40.64, 34.78, 28.46, 27.47. m/z calcd. for C₂₇H₃₂N₈O₃=516.3. Found [M+H]⁺=517.3.

### N-((1s,4s)-4-((7-Morpholino-1,6-naphthyridin-5-yl)oxy)cyclohexyl)-5H-pyrrolo[3,2-d]pyrimidin-2-amine (85)

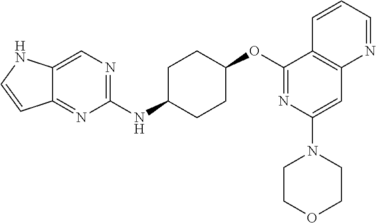 [SMILES: c1cnc2cc(N3CCOCC3)nc(O[C@H]3CC[C@@H](Nc4ncc5[nH]ccc5n4)CC3)c2c1]

Prepared according to general procedure H starting with 2-chloro-5H-pyrrolo[3,2-d]pyrimidine and intermediate 7 to afford the titled compound in 35% yield. ¹H NMR (400 MHz, DMSO-d₆) δ 11.14 (s, 1H), 8.80 (dd, J=4.4, 1.8 Hz, 1H), 8.45 (d, J=0.9 Hz, 1H), 8.40 (dd, 1H), 7.54 (t, J=2.9 Hz, 1H), 7.22 (dd, J=8.2, 4.3 Hz, 1H), 6.53 (s, 1H), 6.25 (d, J=8.1 Hz, 1H), 6.20-6.14 (m, 1H), 5.43 (s, 1H), 3.90 (s, 1H), 3.75 (t, J=4.8 Hz, 4H), 3.50 (t, J=4.9 Hz, 4H), 2.11-2.06 (m, 2H), 1.86-1.75 (m, 6H). ¹³C NMR (100 MHz, DMSO-d₆) δ 158.94, 158.06, 156.96, 155.71, 155.32, 151.71, 141.70, 132.83, 132.58, 122.55, 118.32, 109.12, 99.38, 92.38, 70.79, 66.34, 48.76, 45.83, 28.76, 27.90. m/z calcd. for C₂₄H₂₇N₇O₂=445.2. Found [M+H]⁺=446.2.

### N,N-Dimethyl-2-(2-(((1s,4s)-4-((7-morpholino-1,6-naphthyridin-5-yl)oxy)cyclohexyl)amino)-5H-pyrrolo[3,2-d]pyrimidin-5-yl)acetamide (86)

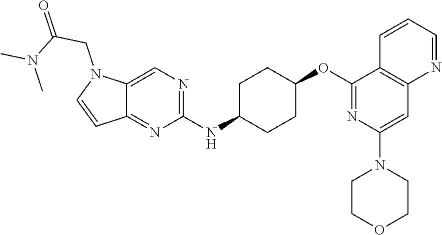 [SMILES: CN(C)C(=O)Cn1ccc2nc(N[C@H]3CC[C@@H](Oc4nc(N5CCOCC5)cc5ncccc45)CC3)ncc21]

Prepared according to general procedure P starting with indole 85 and 2-chloro-N,N-dimethylacetamide to afford the titled compound in 52% yield. ¹H NMR (400 MHz, DMSO-d₆) δ 8.80 (dd, J=4.3, 1.9 Hz, 1H), 8.46 (d, J=0.8 Hz, 1H), 8.40 (dd, J=8.4, 1.8 Hz, 1H), 7.42 (d, J=3.1 Hz, 1H), 7.22 (dd, J=8.2, 4.3 Hz, 1H), 6.53 (s, 1H), 6.35 (d, J=8.1 Hz, 1H), 6.17 (d, J=3.0 Hz, 1H), 5.42 (s, 1H), 5.12 (s, 2H), 3.90 (s, 1H), 3.75 (t, J=4.8 Hz, 4H), 3.50 (t, J=4.9 Hz, 4H), 3.08 (s, 3H), 2.86 (s, 3H), 2.09 (s, 2H), 1.86-1.76 (m, 6H). ¹³C NMR (100 MHz, DMSO-d₆) δ 167.59, 158.94, 158.07, 156.96, 155.69, 155.30, 141.08, 136.66, 132.84, 123.88, 118.31, 109.13, 99.00, 92.36, 70.82, 66.34, 48.73, 48.00, 45.83, 36.31, 35.74, 28.71, 27.85. m/z calcd. for C₂₈H₃₄N₈O₃=530.3. Found [M+H]⁺=531.3.

### Ethyl 4-chloro-2-((tetrahydro-2H-pyran-4-yl)amino)pyrimidine-5-carboxylate (93-i)

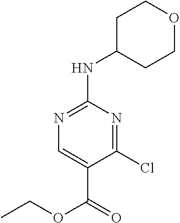 [SMILES: CCOC(=O)c1cnc(NC2CCOCC2)nc1Cl]

Prepared according to J. Med Chem. 2020, 63, 3461 with matching spectroscopic data.

### Ethyl 4-(((1s,4s)-4-((7-morpholino-1,6-naphthyridin-5-yl)oxy)cyclohexyl)amino)-2-((tetrahydro-2H-pyran-4-yl)amino)pyrimidine-5-carboxylate (93)

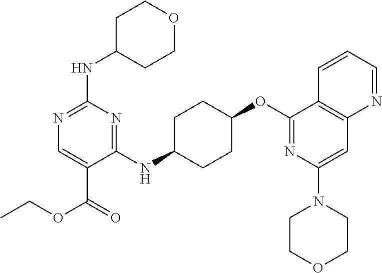 [SMILES: CCOC(=O)c1cnc(NC2CCOCC2)nc1N[C@H]1CC[C@@H](Oc2nc(N3CCOCC3)cc3ncccc23)CC1]

Prepared according to general procedure H starting with 93-i and intermediate 7 to afford the titled compound in 47% yield. ¹H NMR (400 MHz, DMSO-d₆) δ 8.87 (dd, J=5.3, 1.7 Hz, 1H), 8.80 (d, J=8.0 Hz, 1H), 8.55-8.46 (m, 2H), 8.24-8.19 (m, 1H), 7.37 (dd, J=8.1, 5.1 Hz, 1H), 6.49 (s, 1H), 5.48 (s, 1H), 4.26 (q, J=7.1 Hz, 2H), 4.16 (s, 1H), 4.03-3.84 (m, 2H), 3.75 (dd, J=5.8, 3.9 Hz, 4H), 3.66-3.59 (m, 4H), 3.46-3.33 (m, 3H), 2.05 (s, 2H), 1.94-1.73 (m, 8H), 1.63-1.48 (m, 2H), 1.30 (t, J=7.0 Hz, 3H). m/z calcd. for C₃₀H₃₉N₇O₅=577.3. Found [M+H]⁺=578.3.

### Ethyl 2-chloro-4-((tetrahydro-2H-pyran-4-yl)amino)pyrimidine-5-carboxylate (94-i)

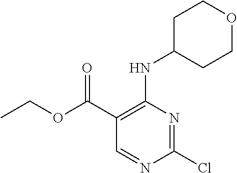 [SMILES: CCOC(=O)c1cnc(Cl)nc1NC1CCOCC1]

Prepared according to J. Med Chem. 2020, 63, 3461 with matching spectroscopic data.

### Ethyl 2-(((1s,4s)-4-((7-morpholino-1,6-naphthyridin-5-yl)oxy)cyclohexyl)amino)-4-((tetrahydro-2H-pyran-4-yl)amino)pyrimidine-5-carboxylate (94)

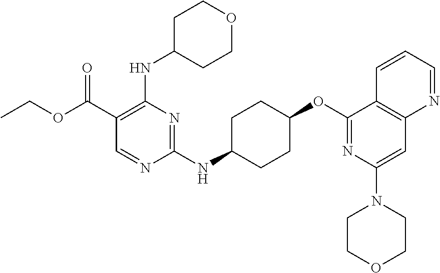 [SMILES: CCOC(=O)c1cnc(N[C@H]2CC[C@@H](Oc3nc(N4CCOCC4)cc4ncccc34)CC2)nc1NC1CCOCC1]

Prepared according to general procedure H starting with 94-i and intermediate 7 to afford the titled compound in 8% yield. ¹H NMR (400 MHz, DMSO-d₆) δ 8.87 (dd, J=5.2, 1.9 Hz, 1H), 8.74-8.59 (m, 1H), 8.54-8.38 (m, 1H), 8.18 (s, 1H), 7.43-7.32 (m, 1H), 6.49 (s, 1H), 5.45 (s, 1H), 4.31-4.17 (m, 3H), 3.93-3.85 (m, 3H), 3.75 (t, J=4.9 Hz, 4H), 3.61 (t, J=5.2 Hz, 4H), 3.49-3.25 (m, 3H), 2.13-2.06 (m, 2H), 2.00-1.68 (m, 8H), 1.67-1.49 (m, 2H), 1.33-1.24 (m, 3H). m/z calcd. for C₃₀H₃₉N₇O₅=577.3. Found [M+H]⁺=578.3.

### 2-Chloro-9-(tetrahydro-2H-pyran-4-yl)-7,9-dihydro-8H-purin-8-one (95-i)

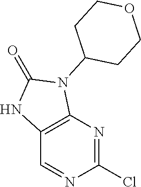 [SMILES: O=c1[nH]c2cnc(Cl)nc2n1C1CCOCC1]

Prepared according to J. Med Chem. 2020, 63, 3461 with matching spectroscopic data.

### 2-(((1s,4s)-4-((7-Morpholino-1,6-naphthyridin-5-yl)oxy)cyclohexyl)amino)-9-(tetrahydro-2H-pyran-4-yl)-7,9-dihydro-8H-purin-8-one (95)

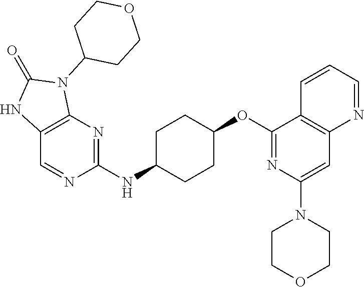 [SMILES: O=c1[nH]c2cnc(N[C@H]3CC[C@@H](Oc4nc(N5CCOCC5)cc5ncccc45)CC3)nc2n1C1CCOCC1]

Prepared according to general procedure H starting with 95-i and intermediate 7 to afford the titled compound in 19% yield. ¹H NMR (400 MHz, DMSO-d₆) δ 11.26 (s, 1H), 8.88 (dd, J=5.2, 1.7 Hz, 1H), 8.65 (d, J=8.0 Hz, 1H), 7.86 (s, 1H), 7.37 (dd, J=8.1, 5.1 Hz, 1H), 6.50 (s, 1H), 5.47 (s, 1H), 4.43-4.30 (m, 1H), 3.98 (dd, J=11.2, 4.4 Hz, 2H), 3.88-3.82 (m, 1H), 3.75 (t, J=4.8 Hz, 4H), 3.65-3.58 (m, 4H), 3.47-3.37 (m, 5H), 2.11 (s, 2H), 1.82 (d, J=7.4 Hz, 6H), 1.67 (d, J=12.4 Hz, 2H). m/z calcd. for C₂₈H₃₄N₈O₄=546.3. Found [M+H]⁺=547.2.

### 2-Chloro-7-methyl-9-(tetrahydro-2H-pyran-4-yl)-7,9-dihydro-8H-purin-8-one (96-i)

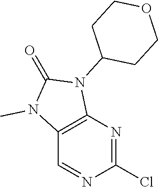 [SMILES: Cn1c(=O)n(C2CCOCC2)c2nc(Cl)ncc21]

Prepared according to J. Med Chem. 2020, 63, 3461 with matching spectroscopic data.

7-Methyl-2-(((1s,4s)-4-((7-morpholino-1,6-naphthyridin-5-yl)oxy)cyclohexyl)amino)-9-(tetrahydro-2H-pyran-4-yl)-7,9-dihydro-8H-purin-8-one (96)

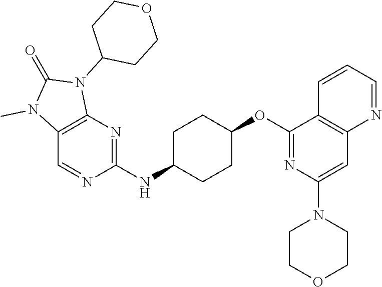 [SMILES: Cn1c(=O)n(C2CCOCC2)c2nc(N[C@H]3CC[C@@H](Oc4nc(N5CCOCC5)cc5ncccc45)CC3)ncc21]

Prepared according to general procedure H starting with 96-i and intermediate 7 to afford the titled compound in 28% yield. ¹H NMR (400 MHz, DMSO-d₆) δ 8.88 (dd, J=5.2, 1.7 Hz, 1H), 8.67 (d, J=8.1 Hz, 1H), 8.03 (s, 1H), 7.37 (dd, J=8.0, 5.1 Hz, 1H), 6.50 (s, 1H), 5.47 (s, 1H), 4.45-4.33 (m, 1H), 3.98 (dd, J=11.2, 4.4 Hz, 2H), 3.86 (s, 1H), 3.75 (t, J=4.8 Hz, 4H), 3.61 (t, J=4.9 Hz, 4H), 3.48-3.34 (m, 5H), 3.28 (s, 3H), 2.13-2.09 (m, 2H), 1.85-1.77 (m, 6H), 1.71-1.63 (m, 2H). m/z calcd. for C₂₉H₃₆N₈O₄=560.3. Found [M+H]⁺=561.3.

### 2-(2-Chloro-8-oxo-9-(tetrahydro-2H-pyran-4-yl)-8,9-dihydro-7H-purin-7-yl)-N,N-dimethylacetamide (97-i)

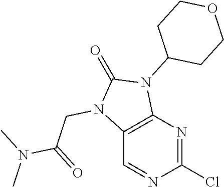 [SMILES: CN(C)C(=O)Cn1c(=O)n(C2CCOCC2)c2nc(Cl)ncc21]

Prepared according to general procedure P starting with 95-i and 2-chloro-N,N-dimethylacetamide to afford the titled compound in 60% yield. ¹H NMR (400 MHz, DMSO-d₆) δ 8.32 (s, 1H), 4.85 (s, 2H), 4.54-4.44 (m, 1H), 4.06-3.94 (m, 2H), 3.47 (t, J=11.9 Hz, 2H), 3.07 (s, 3H), 2.85 (s, 3H), 2.44 (td, J=12.6, 4.8 Hz, 2H), 1.70 (d, J=11.9 Hz, 2H). m/z calcd. for C₁₄H₁₈ClN₅O₃=339.1. Found [M+H]⁺=340.1.

### N,N-Dimethyl-2-(2-(((1s,4s)-4-((7-morpholino-1,6-naphthyridin-5-yl)oxy)cyclohexyl)amino)-8-oxo-9-(tetrahydro-2H-pyran-4-yl)-8,9-dihydro-7H-purin-7-yl)acetamide (97)

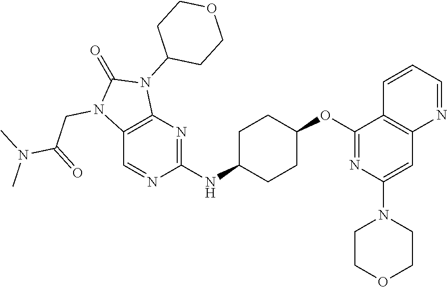 [SMILES: CN(C)C(=O)Cn1c(=O)n(C2CCOCC2)c2nc(N[C@H]3CC[C@@H](Oc4nc(N5CCOCC5)cc5ncccc45)CC3)ncc21]

Prepared according to general procedure H starting with 97-i and intermediate 7 to afford the titled compound in 38% yield. ¹H NMR (400 MHz, DMSO-d₆) δ 8.80 (dd, J=4.3, 1.8 Hz, 1H), 8.39 (dd, J=8.2, 1.9 Hz, 1H), 7.87 (s, 1H), 7.23 (dd, J=8.2, 4.3 Hz, 1H), 6.78 (d, J=7.8 Hz, 1H), 6.53 (s, 1H), 5.42 (s, 1H), 4.68 (s, 2H), 4.43-4.31 (m, 1H), 3.98 (dd, J=11.4, 4.4 Hz, 2H), 3.84 (s, 1H), 3.75 (t, J=4.8 Hz, 4H), 3.50 (t, J=4.9 Hz, 4H), 3.44 (d, J=11.9 Hz, 2H), 3.06 (s, 3H), 2.84 (s, 3H), 2.58 (td, J=12.5, 4.6 Hz, 2H), 2.08 (s, 2H), 1.82-1.73 (m, 6H), 1.63 (d, J=12.2 Hz, 2H). m/z calcd. for C₃₂H₄₁N₉O₅=631.3. Found [M+H]⁺=632.3.

### 2-Chloro-7-(2-morpholino-2-oxoethyl)-9-(tetrahydro-2H-pyran-4-yl)-7,9-dihydro-8H-purin-8-one (98-i)

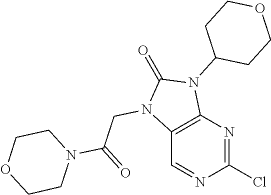 [SMILES: O=C(Cn1c(=O)n(C2CCOCC2)c2nc(Cl)ncc21)N1CCOCC1]

Prepared according to general procedure P starting with 95-i and 2-chloro-1-morpholinoethan-1-one to afford the titled compound in 69% yield. ¹H NMR (400 MHz, DMSO-d₆) δ 8.33 (s, 1H), 4.90 (s, 2H), 4.49 (tt, J=12.1, 4.2 Hz, 1H), 3.98 (dd, J=11.5, 4.5 Hz, 2H), 3.68 (t, J=4.7 Hz, 2H), 3.59 (t, J=4.9 Hz, 2H), 3.54 (t, J=4.7 Hz, 2H), 3.52-3.46 (m, 2H), 3.46-3.39 (m, 2H), 2.44 (td, J=12.4, 4.7 Hz, 2H), 1.74-1.65 (m, 2H). m/z calcd. for C₁₆H₂₀ClN₅O₄=381.1. Found [M+H]⁺=382.1.

### 2-(((1s,4s)-4-((7-Morpholino-1,6-naphthyridin-5-yl)oxy)cyclohexyl)amino)-7-(2-morpholino-2-oxoethyl)-9-(tetrahydro-2H-pyran-4-yl)-7,9-dihydro-8H-purin-8-one (98)

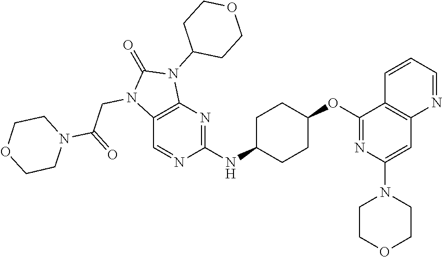 [SMILES: O=C(Cn1c(=O)n(C2CCOCC2)c2nc(N[C@H]3CC[C@@H](Oc4nc(N5CCOCC5)cc5ncccc45)CC3)ncc21)N1CCOCC1]

Prepared according to general procedure H starting with 98-i and intermediate 7 to afford the titled compound in 32% yield. ¹H NMR (400 MHz, DMSO-d₆) δ 8.81 (dd, J=4.3, 1.8 Hz, 1H), 8.43-8.36 (m, 1H), 7.88 (s, 1H), 7.23 (dd, J=8.2, 4.4 Hz, 1H), 6.80 (d, J=7.9 Hz, 1H), 6.53 (s, 1H), 5.42 (s, 1H), 4.72 (s, 2H), 4.42-4.31 (m, 1H), 3.98 (d, J=11.2 Hz, 2H), 3.84 (s, 1H), 3.75 (t, J=4.8 Hz, 4H), 3.65 (d, J=5.0 Hz, 2H), 3.61-3.46 (m, 8H), 3.41 (d, J=11.7 Hz, 4H), 2.56 (dd, J=12.4, 4.6 Hz, 2H), 2.08 (s, 2H), 1.82-1.73 (m, 6H), 1.63 (d, J=12.2 Hz, 2H). m/z calcd. for C₃₄H₄₃N₉O₆=673.3. Found [M+H]⁺=674.3.

### 7-(2-(Azetidin-1-yl)-2-oxoethyl)-2-chloro-9-(tetrahydro-2H-pyran-4-yl)-7,9-dihydro-8H-purin-8-one (99-i)

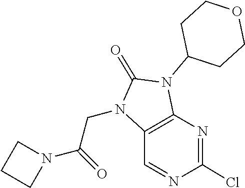 [SMILES: O=C(Cn1c(=O)n(C2CCOCC2)c2nc(Cl)ncc21)N1CCC1]

Prepared according to general procedure P starting with 95-i and 1-(azetidin-1-yl)-2-chloroethan-1-one to afford the titled compound in 75% yield. ¹H NMR (400 MHz, DMSO-d₆) δ 8.33 (s, 1H), 4.59 (s, 2H), 4.54-4.43 (m, 1H), 4.27 (t, J=7.7 Hz, 2H), 3.98 (dd, J=11.5, 4.5 Hz, 2H), 3.91 (t, J=7.7 Hz, 2H), 3.47 (t, J=11.9 Hz, 2H), 2.43 (td, J=12.5, 4.6 Hz, 2H), 2.29 (td, J=13.8, 6.1 Hz, 2H), 1.69 (d, J=11.7 Hz, 2H). m/z calcd. for C₁₅H₁₈ClN₅O₃=351.1. Found [M+H]⁺=352.1.

### 7-(2-(Azetidin-1-yl)-2-oxoethyl)-2-(((1s,4s)-4-((7-morpholino-1,6-naphthyridin-5-yl)oxy)cyclohexyl)amino)-9-(tetrahydro-2H-pyran-4-yl)-7,9-dihydro-8H-purin-8-one (99)

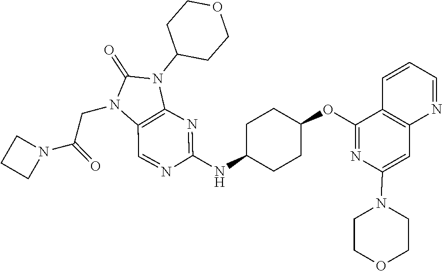 [SMILES: O=C(Cn1c(=O)n(C2CCOCC2)c2nc(N[C@H]3CC[C@@H](Oc4nc(N5CCOCC5)cc5ncccc45)CC3)ncc21)N1CCC1]

Prepared according to general procedure H starting with 99-i and intermediate 7 to afford the titled compound in 28% yield. ¹H NMR (400 MHz, DMSO-d₆) δ 8.80 (dd, J=4.3, 1.8 Hz, 1H), 8.39 (dd, J=8.1, 1.8 Hz, 1H), 7.88 (s, 1H), 7.23 (dd, J=8.2, 4.3 Hz, 1H), 6.81 (d, J=7.9 Hz, 1H), 6.53 (s, 1H), 5.42 (s, 1H), 4.42 (s, 2H), 4.39-4.30 (m, 1H), 4.24 (t, J=7.6 Hz, 2H), 4.01-3.94 (m, 2H), 3.90 (t, J=7.7 Hz, 2H), 3.85 (s, 1H), 3.75 (t, J=4.9 Hz, 4H), 3.50 (t, J=4.9 Hz, 4H), 3.47-3.38 (m, 2H), 2.62-2.51 (m, 2H), 2.27 (p, J=7.6 Hz, 2H), 2.08 (s, 2H), 1.82-1.73 (m, 6H), 1.63 (d, J=12.2 Hz, 2H). m/z calcd. for C₃₃H₄₁N₉O₅=643.3. Found [M+H]⁺=644.3.

### 9-Methyl-2-(((1s,4s)-4-((7-morpholino-1,6-naphthyridin-5-yl)oxy)cyclohexyl)amino)-7,9-dihydro-8H-purin-8-one (100)

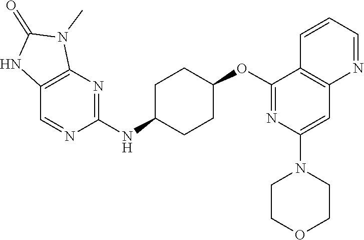 [SMILES: Cn1c(=O)[nH]c2cnc(N[C@H]3CC[C@@H](Oc4nc(N5CCOCC5)cc5ncccc45)CC3)nc21]

Prepared according to general procedure H starting with commercially available 2-chloro-9-methyl-7,9-dihydro-8H-purin-8-one and intermediate 7 to afford the titled compound in 16% yield. ¹H NMR (400 MHz, DMSO-d₆) δ 11.23 (s, 1H), 8.86 (dd, J=5.0, 1.7 Hz, 1H), 8.60 (d, J=8.1 Hz, 1H), 7.89 (s, 1H), 7.84 (s, 1H), 7.34 (dd, J=8.1, 5.0 Hz, 1H), 6.51 (s, 1H), 5.45 (s, 1H), 3.90 (s, 1H), 3.75 (dd, J=5.8, 3.9 Hz, 4H), 3.59 (t, J=4.9 Hz, 4H), 3.23 (s, 3H), 2.14-2.06 (m, 2H), 1.88-1.77 (m, 6H). m/z calcd. for C₂₄H₂₈N₈O₃=476.2. Found [M+H]⁺=477.2.

### 2-Chloro-7,9-dimethyl-7,9-dihydro-8H-purin-8-one (101-i)

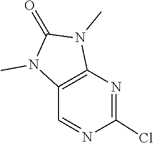

Prepared according to general procedure P starting with commercially available 2-chloro-9-methyl-7,9-dihydro-8H-purin-8-one and methyl iodide to afford the titled compound in 92% yield. ¹H NMR (400 MHz, DMSO-d₆) δ 8.34 (s, 1H), 3.36 (s, 3H), 3.31 (s, 3H). m/z calcd. for C₇H₇ClN₄O=198.0. Found [M+H]⁺=199.0.

### 7,9-Dimethyl-2-(((1s,4s)-4-((7-morpholino-1,6-naphthyridin-5-yl)oxy)cyclohexyl)amino)-7,9-dihydro-8H-purin-8-one (101)

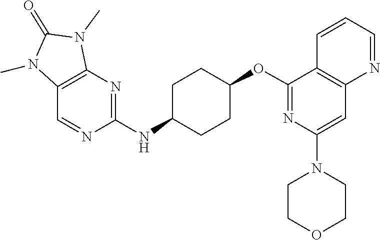 [SMILES: Cn1c(=O)n(C)c2nc(N[C@H]3CC[C@@H](Oc4nc(N5CCOCC5)cc5ncccc45)CC3)ncc21]

Prepared according to general procedure H starting with 101-i and intermediate 7 to afford the titled compound in 26% yield. ¹H NMR (400 MHz, DMSO-d₆) δ 8.88 (dd, J=5.2, 1.7 Hz, 1H), 8.67 (d, J=8.0 Hz, 1H), 8.19-7.88 (m, 2H), 7.37 (dd, J=8.1, 5.2 Hz, 1H), 6.50 (s, 1H), 5.46 (s, 1H), 3.90 (s, 1H), 3.75 (dd, J=5.8, 3.9 Hz, 4H), 3.61 (t, J=4.9 Hz, 4H), 3.29 (s, 3H), 3.26 (s, 3H), 2.14-2.10 (m, 2H), 1.88-1.77 (m, 6H). m/z calcd. for C₂₅H₃₀N₈O₃=490.2. Found [M+H]⁺=491.2.

### 2-(2-Chloro-9-methyl-8-oxo-8,9-dihydro-7H-purin-7-yl)-N,N-dimethylacetamide (102-i)

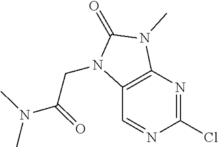 [SMILES: CN(C)C(=O)Cn1c(=O)n(C)c2nc(Cl)ncc21]

Prepared according to general procedure P starting with commercially available 2-chloro-9-methyl-7,9-dihydro-8H-purin-8-one and 2-chloro-N,N-dimethylacetamide to afford the titled compound in 63% yield. ¹H NMR (400 MHz, DMSO-d₆) δ 8.29 (s, 1H), 4.86 (s, 2H), 3.36 (s, 3H), 3.07 (s, 3H), 2.85 (s, 3H). m/z calcd. for C₁₀H₁₂ClN₅O₂=269.1. Found [M+H]⁺=270.0.

### N,N-Dimethyl-2-(9-methyl-2-(((1s,4s)-4-((7-morpholino-1,6-naphthyridin-5-yl)oxy)cyclohexyl)amino)-8-oxo-8,9-dihydro-7H-purin-7-yl)acetamide (102)

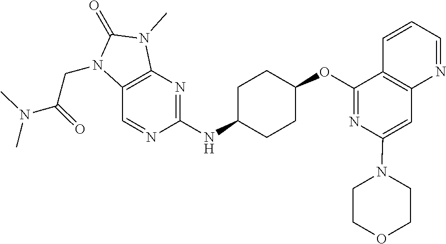

Prepared according to general procedure H starting with 102-i and intermediate 7 to afford the titled compound in 32% yield. ¹H NMR (400 MHz, DMSO-d₆) δ 8.80 (dd, J=4.3, 1.8 Hz, 1H), 8.39 (dd, J=8.5, 1.8 Hz, 1H), 7.85 (s, 1H), 7.22 (dd, J=8.2, 4.3 Hz, 1H), 6.80 (d, J=8.0 Hz, 1H), 6.53 (s, 1H), 5.41 (s, 1H), 4.69 (s, 2H), 3.86 (s, 1H), 3.75 (t, J=4.8 Hz, 4H), 3.50 (t, J=4.9 Hz, 4H), 3.24 (s, 3H), 3.06 (s, 3H), 2.84 (s, 3H), 2.10-2.06 (m, 2H), 1.81-1.73 (m, 6H). m/z calcd. for C₂₈H₃₅N₉O₄=561.3. Found [M+H]⁺=562.3.

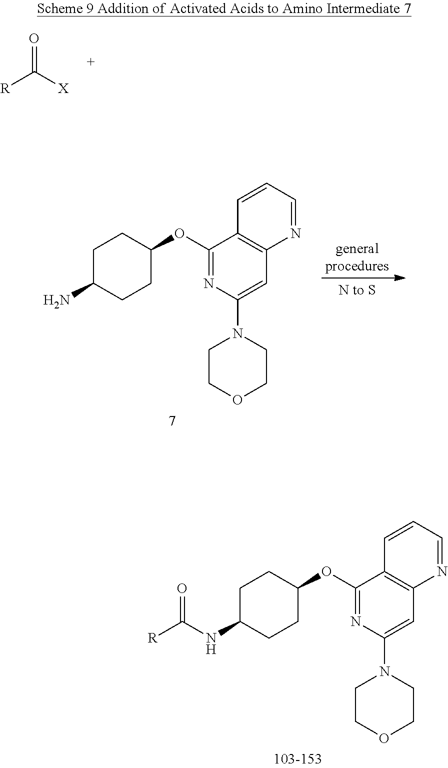

### 2-Methoxy-N-((1s,4s)-4-((7-morpholino-1,6-naphthyridin-5-yl)oxy)cyclohexyl)acetamide (105)

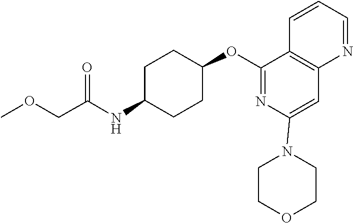 [SMILES: COCC(=O)N[C@H]1CC[C@@H](Oc2nc(N3CCOCC3)cc3ncccc23)CC1]

Prepared according to general procedure Q starting with 2-methoxyacetyl chloride and intermediate 7 to afford the titled compound in 70% yield. ¹H NMR (400 MHz, DMSO-d₆) δ 8.80 (dd, J=4.3, 1.8 Hz, 1H), 8.39 (dd, J=8.2, 1.8 Hz, 1H), 7.71 (d, J=8.3 Hz, 1H), 7.22 (dd, J=8.2, 4.3 Hz, 1H), 6.52 (s, 1H), 5.40 (s, 1H), 3.80 (s, 3H), 3.74 (dd, J=5.7, 4.0 Hz, 4H), 3.49 (t, J=4.8 Hz, 4H), 3.32 (s, 3H), 2.09-2.01 (m, 2H), 1.86-1.67 (m, 4H), 1.65-1.58 (m, 2H). ¹³C NMR (100 MHz, DMSO-d₆) δ 168.34, 158.83, 156.93, 155.71, 155.32, 132.81, 118.28, 109.06, 92.40, 72.02, 70.07, 66.34, 58.90, 46.71, 45.83, 28.65, 27.42. m/z calcd. for C₂₁H₂₈N₄O₄=400.2. Found [M+H]⁺=401.3.

### 2-(Dimethylamino)-N-((1s,4s)-4-((7-morpholino-1,6-naphthyridin-5-yl)oxy)cyclohexyl)acetamide (106)

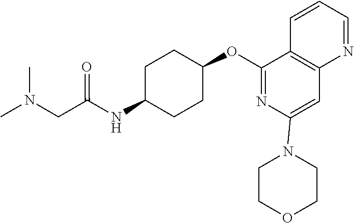 [SMILES: CN(C)CC(=O)N[C@H]1CC[C@@H](Oc2nc(N3CCOCC3)cc3ncccc23)CC1]

Prepared according to general procedure O starting with dimethylglycine and intermediate 7 to afford the titled compound in 64% yield. ¹H NMR (400 MHz, DMSO-d₆) δ 8.80 (dd, J=4.4, 1.8 Hz, 1H), 8.39 (dd, J=8.2, 1.8 Hz, 1H), 7.71 (d, J=8.4 Hz, 1H), 7.22 (dd, J=8.2, 4.3 Hz, 1H), 6.52 (s, 1H), 5.39 (s, 1H), 3.74 (t, J=4.9 Hz, 5H), 3.49 (t, J=4.9 Hz, 4H), 2.93 (s, 2H), 2.25 (s, 6H), 2.07-2.00 (m, 2H), 1.82-1.68 (m, 4H), 1.68-1.61 (m, 2H). ¹³C NMR (100 MHz, DMSO-d₆) δ 168.57, 158.83, 156.92, 155.70, 155.31, 132.81, 118.30, 109.04, 92.41, 70.23, 66.34, 62.98, 46.68, 45.83, 45.70, 28.60, 27.58. m/z calcd. for C₂₂H₃₁N₅O₃=413.2. Found [M+H]⁺=414.2.

### 2-Cyano-N-((1s,4s)-4-((7-morpholino-1,6-naphthyridin-5-yl)oxy)cyclohexyl)acetamide (107)

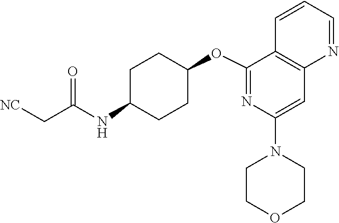 [SMILES: N#CCC(=O)N[C@H]1CC[C@@H](Oc2nc(N3CCOCC3)cc3ncccc23)CC1]

Prepared according to general procedure 0 starting with 2-cyanoacetic acid and intermediate 7 to afford the titled compound in 24% yield. ¹H NMR (400 MHz, DMSO-d₆) δ 8.80 (dd, J=4.3, 1.8 Hz, 1H), 8.32-8.23 (m, 2H), 7.21 (dd, J=8.2, 4.3 Hz, 1H), 6.53 (s, 1H), 5.34 (d, J=4.9 Hz, 1H), 3.74 (t, J=4.9 Hz, 5H), 3.61 (s, 2H), 3.49 (t, J=4.9 Hz, 4H), 2.08-1.99 (m, 2H), 1.84-1.59 (m, 6H). ¹³C NMR (100 MHz, DMSO-d₆) δ 161.63, 158.74, 156.92, 155.71, 155.35, 132.41, 118.32, 116.78, 108.91, 92.45, 70.65, 66.34, 47.37, 45.82, 28.18, 27.53, 25.86. m/z calcd. for C₂₁H₂₅N₅O₃=395.2. Found [M+H]⁺=396.4.

### 117: 6-(2-(dimethylamino)-2-oxoethoxy)-N-((1s,4s)-4-((7-morpholino-1,6-naphthyridin-5-yl)oxy)cyclohexyl)nicotinamide

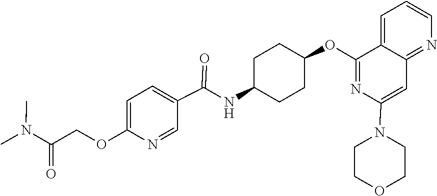 [SMILES: CN(C)C(=O)COc1ccc(C(=O)N[C@H]2CC[C@@H](Oc3nc(N4CCOCC4)cc4ncccc34)CC2)cn1]

Prepared according to general procedures 0 and R starting with 6-chloronicotinic acid and intermediate 7 and then subsequently with 2-hydroxy-N,N-dimethylacetamide to afford the titled compound in 34% yield over 2 steps. ¹H NMR (400 MHz, Chloroform-d) δ 8.79 (dd, J=4.4, 1.9 Hz, 1H), 8.52 (d, J=2.4 Hz, 1H), 8.33 (dd, J=8.2, 1.8 Hz, 1H), 8.01 (dd, J=8.6, 2.5 Hz, 1H), 7.10 (dd, J=8.2, 4.4 Hz, 1H), 6.93 (d, J=8.7 Hz, 1H), 6.54 (s, 1H), 6.04 (d, J=7.9 Hz, 1H), 5.54-5.42 (m, 1H), 5.06 (s, 2H), 4.21-4.06 (m, 1H), 3.86 (t, J=4.9 Hz, 4H), 3.54 (t, J=4.9 Hz, 4H), 3.07 (s, 3H), 2.98 (s, 3H), 2.29-2.13 (m, 2H), 2.02-1.92 (m, 2H), 1.90-1.65 (m, 6H). ¹³C NMR (101 MHz, Chloroform-d) δ 167.63, 165.07, 164.72, 158.96, 157.11, 155.73, 154.72, 146.09, 138.06, 132.86, 124.76, 117.73, 111.20, 109.87, 92.76, 69.99, 66.82, 63.39, 47.96, 46.01, 36.17, 35.75, 28.74, 28.11.

### 125: 5-(2-(dimethylamino)-2-oxoethoxy)-N-((1s,4s)-4-((7-morpholinopyrido[4,3-d]pyrimidin-5-yl)oxy)cyclohexyl)pyrimidine-2-carboxamide

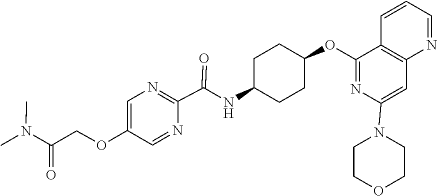 [SMILES: CN(C)C(=O)COc1cnc(C(=O)N[C@H]2CC[C@@H](Oc3nc(N4CCOCC4)cc4ncccc34)CC2)nc1]

Prepared according to general procedures O and R starting with 5-chloropyrimidine-2-carboxylic acid and intermediate 7 and then subsequently with 2-hydroxy-N,N-dimethylacetamide to afford the titled compound in 35% yield over 2 steps. ¹H NMR (400 MHz, Methanol-d₄) δ 8.71 (dd, J=4.5, 1.8 Hz, 1H), 8.59 (s, 2H), 8.55 (dd, J=8.3, 1.8 Hz, 1H), 7.22 (dd, J=8.2, 4.5 Hz, 1H), 6.48 (s, 1H), 5.55-5.47 (m, 1H), 5.15 (s, 2H), 4.14-4.03 (m, 1H), 3.84 (t, J=4.9 Hz, 4H), 3.58 (t, J=4.8 Hz, 4H), 3.09 (s, 3H), 2.99 (s, 3H), 2.31-2.20 (m, 2H), 2.06-1.83 (m, 6H). ¹³C NMR (101 MHz, Chloroform-d) δ 165.68, 158.89, 156.99, 155.62, 154.61, 153.26, 151.18, 143.99, 132.81, 117.63, 109.78, 92.63, 70.01, 66.78, 66.71, 47.35, 45.90, 36.21, 35.74, 28.56, 27.87.

### 126: 5-(2-(dimethylamino)ethoxy)-N-((1s,4s)-4-((7-morpholino-1,6-naphthyridin-5-yl)oxy)cyclohexyl)pyrimidine-2-carboxamide

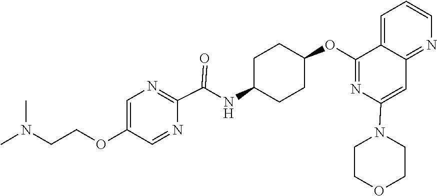 [SMILES: CN(C)CCOc1cnc(C(=O)N[C@H]2CC[C@@H](Oc3nc(N4CCOCC4)cc4ncccc34)CC2)nc1]

Prepared according to general procedure O and R starting with 5-chloropyrimidine-2-carboxylic acid and intermediate 7 and then subsequently with 2-dimethylaminoethanol to afford the titled compound in 13% yield over 2 steps. ¹H NMR (400 MHz, Methanol-d₄) δ 8.74 (s, 1H), 8.66 (s, 2H), 8.56 (dd, J=8.2, 1.7 Hz, 1H), 7.25 (dd, J=8.2, 4.5 Hz, 1H), 6.50 (s, 1H), 5.59-5.46 (m, 1H), 4.41 (t, J=5.2 Hz, 2H), 4.19-4.02 (m, 1H), 3.86 (t, J=4.9 Hz, 4H), 3.60 (t, J=4.9 Hz, 4H), 2.98 (s, 2H), 2.60-2.39 (m, 6H), 2.27 (d, J=13.5 Hz, 2H), 2.11-1.83 (m, 6H). ¹³C NMR (101 MHz, Methanol-d₄) δ 163.74, 160.28, 158.68, 156.31, 155.25, 152.05, 145.08, 135.12, 118.78, 111.06, 91.73, 71.54, 67.72, 66.22, 58.12, 49.28, 49.07, 46.91, 44.89, 29.67, 28.24.

### 128: 5-methyl-N-((1s,4s)-4-((7-morpholino-1,6-naphthyridin-5-yl)oxy)cyclohexyl)pyrimidine-2-carboxamide

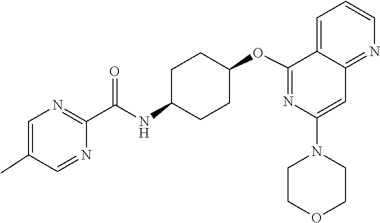 [SMILES: Cc1cnc(C(=O)N[C@H]2CC[C@@H](Oc3nc(N4CCOCC4)cc4ncccc34)CC2)nc1]

Prepared according to general procedure O starting with 5-methylpyrimidine-2-carboxylic acid and intermediate 7 to afford the titled compound in 55% yield. ¹H NMR (400 MHz, DMSO-d₆) δ 8.93-8.75 (m, 4H), 8.70 (d, J=8.5 Hz, 1H), 7.40 (dd, J=8.0, 5.4 Hz, 1H), 6.47 (s, 1H), 5.54-5.44 (m, 1H), 3.74 (t, J=4.8 Hz, 4H), 3.63 (t, J=4.8 Hz, 4H), 2.35 (s, 3H), 2.14-2.03 (m, 2H), 2.02-1.88 (m, 2H), 1.87-1.63 (m, 4H). ¹³C NMR (101 MHz, DMSO-d₆) δ 161.63, 159.19, 158.24 (q, J=33.3 Hz, TFA), 157.94, 157.46, 156.00, 149.16, 148.66, 140.11, 116.42, 110.54, 84.46, 71.06, 65.86, 47.30, 45.09, 28.15, 26.70, 15.26.

### 129: 1,1,3,3-tetramethyl-2-((1s,4s)-4-((7-morpholino-1,6-naphthyridin-5-yl)oxy)cyclohexyl)guanidine

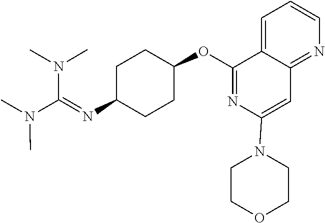 [SMILES: CN(C)C(=N[C@H]1CC[C@@H](Oc2nc(N3CCOCC3)cc3ncccc23)CC1)N(C)C]

Compound 129 was obtained as the by-product when preparing compound 128. ¹H NMR (400 MHz, DMSO-d₆) δ 8.85 (dd, J=5.1, 1.4 Hz, 1H), 8.59 (d, J=7.9 Hz, 1H), 7.59 (d, J=8.8 Hz, 1H), 7.33 (dd, J=8.1, 5.0 Hz, 1H), 6.52 (s, 1H), 5.41 (s, 1H), 3.74 (t, J=4.8 Hz, 4H), 3.57 (t, J=4.9 Hz, 4H), 2.93 (s, 12H), 2.11 (d, J=10.9 Hz, 2H), 1.90-1.69 (m, 6H). ¹³C NMR (101 MHz, DMSO-d₆) δ 160.56, 158.88, 158.14 (q, J=32.9, 32.3 Hz, TFA), 157.48, 150.91, 150.54, 137.55, 116.85, 109.85, 86.72, 70.36, 65.86, 52.84, 45.17, 28.02, 27.76.

### 141: 1-(2-(dimethylamino)-2-oxoethyl)-N-((1s,4s)-4-((7-morpholino-1,6-naphthyridin-5-yl)oxy)cyclohexyl)-1H-pyrrole-3-carboxamide

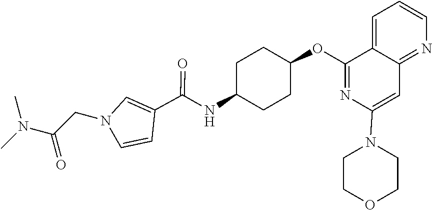 [SMILES: CN(C)C(=O)Cn1ccc(C(=O)N[C@H]2CC[C@@H](Oc3nc(N4CCOCC4)cc4ncccc34)CC2)c1]

Prepared according to general procedures O and P starting with pyrrole-3-carboxylic acid, added HOAt (1.3 eq) and intermediate 7 and then subsequently with 2-chloro-N,N-dimethylacetamide to afford the titled compound in 16% yield over 2 steps. ¹H NMR (400 MHz, Methanol-d₄) δ 8.71 (dd, J=4.5, 1.8 Hz, 1H), 8.52 (dd, J=8.2, 1.8 Hz, 1H), 7.80 (d, J=8.1 Hz, 1H), 7.28 (t, J=2.0 Hz, 1H), 7.22 (dd, J=8.2, 4.5 Hz, 1H), 6.67 (t, J=2.6 Hz, 1H), 6.57 (dd, J=3.0, 1.8 Hz, 1H), 6.48 (s, 1H), 5.54-5.45 (m, 1H), 4.92 (s, 2H), 4.00 (s, 1H), 3.89-3.78 (m, 4H), 3.63-3.51 (m, 4H), 3.09 (s, 3H), 2.97 (s, 3H), 2.30-2.18 (m, 2H), 1.95-1.79 (m, 6H). ¹³C NMR (101 MHz, Methanol-d₄) δ 169.81, 167.05, 160.29, 158.71, 156.32, 155.25, 135.02, 126.44, 124.31, 120.72, 118.70, 111.04, 108.74, 91.66, 71.54, 67.72, 51.63, 48.92, 46.90, 36.74, 36.14, 29.86, 28.45.

### 146: 1-(2-(dimethylamino)-2-oxoethyl)-N-((1s,4s)-4-((7-morpholino-1,6-naphthyridin-5-yl)oxy)cyclohexyl)-1H-pyrrole-2-carboxamide

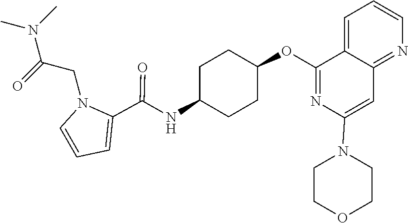 [SMILES: CN(C)C(=O)Cn1cccc1C(=O)N[C@H]1CC[C@@H](Oc2nc(N3CCOCC3)cc3ncccc23)CC1]

Prepared according to general procedure O and P starting with 1H-pyrrole-2-carboxylic acid and intermediate 7 and then subsequently with 2-chloro-N,N-dimethylacetamide to afford the titled compound in 7% yield over 2 steps. ¹H NMR (400 MHz, DMSO-d₆) δ 8.89 (dd, J=5.4, 1.6 Hz, 1H), 8.71 (d, J=7.7 Hz, 1H), 7.78 (d, J=8.0 Hz, 1H), 7.41 (dd, J=8.0, 5.4 Hz, 1H), 6.84 (dd, J=3.9, 1.8 Hz, 1H), 6.81 (t, J=2.2 Hz, 1H), 6.48 (s, 1H), 6.01 (dd, J=3.8, 2.6 Hz, 1H), 5.44 (s, 1H), 5.21 (s, 2H), 3.86-3.80 (m, 1H, overlap with water peak), 3.78-3.70 (m, 4H, overlap with water), 3.63 (t, J=4.9 Hz, 4H, overlap with water), 3.02 (s, 3H), 2.81 (s, 3H), 2.17-2.04 (m, 2H), 1.85-1.59 (m, 6H). ¹³C NMR (101 MHz, DMSO-d₆) δ 167.80, 160.60, 159.10, 157.81, 149.42, 139.30, 128.16, 125.37, 116.41, 111.96, 110.30, 106.30, 71.37, 65.79, 49.65, 46.37, 45.02, 39.94, 35.81, 35.17, 28.21, 27.14.

### 1-((1s,4s)-4-((7-Morpholino-1,6-naphthyridin-5-yl)oxy)cyclohexyl)-3-phenylurea (150)

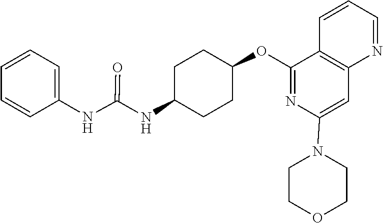 [SMILES: O=C(Nc1ccccc1)N[C@H]1CC[C@@H](Oc2nc(N3CCOCC3)cc3ncccc23)CC1]

Prepared according to general procedure S starting with isocyanatobenzene and intermediate 7 to afford the titled compound in 70% yield. ¹H NMR (400 MHz, DMSO-d₆) δ 8.80 (dd, J=4.3, 1.8 Hz, 1H), 8.34-8.27 (m, 2H), 7.42-7.35 (m, 2H), 7.22 (ddd, J=8.6, 6.0, 1.7 Hz, 3H), 6.89 (tt, J=7.3, 1.2 Hz, 1H), 6.54 (s, 1H), 6.29 (d, J=7.8 Hz, 1H), 5.34 (s, 1H), 3.75 (t, J=4.8 Hz, 4H), 3.72 (s, 1H), 3.50 (t, J=4.9 Hz, 4H), 2.05-1.96 (m, 2H), 1.90-1.75 (m, 4H), 1.71-1.59 (m, 2H). ¹³C NMR (100 MHz, DMSO-d₆) δ 158.80, 156.92, 155.73, 155.34, 154.96, 140.99, 132.47, 129.12, 121.40, 118.35, 117.98, 108.95, 92.48, 71.23, 66.35, 46.63, 45.84, 28.56, 28.23. m/z calcd. for C₂₅H₂₉N₅O₃=447.2. Found [M+H]⁺=448.2.

### 1-(4-Fluorophenyl)-3-((1s,4s)-4-((7-morpholino-1,6-naphthyridin-5-yl)oxy)cyclohexyl)urea (151)

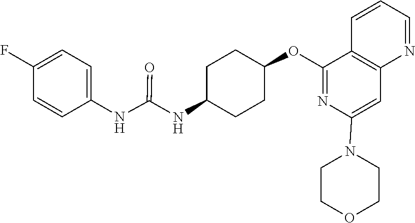 [SMILES: O=C(Nc1ccc(F)cc1)N[C@H]1CC[C@@H](Oc2nc(N3CCOCC3)cc3ncccc23)CC1]

Prepared according to general procedure S starting with 1-fluoro-4-isocyanatobenzene and intermediate 7 to afford the titled compound in 70% yield. ¹H NMR (400 MHz, DMSO-d₆) δ 8.80 (dd, J=4.4, 1.8 Hz, 1H), 8.36-8.27 (m, 2H), 7.44-7.34 (m, 2H), 7.22 (dd, J=8.2, 4.3 Hz, 1H), 7.11-7.00 (m, 2H), 6.54 (s, 1H), 6.28 (d, J=7.8 Hz, 1H), 5.34 (s, 1H), 3.75 (t, J=4.9 Hz, 4H), 3.72-3.61 (m, 1H), 3.50 (t, J=4.9 Hz, 4H), 2.03-1.96 (m, 2H), 1.89-1.74 (m, 4H), 1.71-1.61 (m, 2H). ¹³C NMR (100 MHz, DMSO-d₆) δ 158.80, 157.29 (d, J=237.0 Hz), 156.92, 155.73, 155.34, 155.01, 137.37 (d, J=2.2 Hz), 132.47, 119.55 (d, J=7.5 Hz), 118.35, 115.56 (d, J=22.0 Hz), 108.95, 92.48, 71.17, 66.34, 46.71, 45.84, 28.53, 28.24. m/z calcd. for C₂₅H₂₈FN₅O₃=465.2. Found [M+H]⁺=466.4.

### 1-(4-Methoxyphenyl)-3-((1s,4s)-4-((7-morpholino-1,6-naphthyridin-5-yl)oxy)cyclohexyl)urea (152)

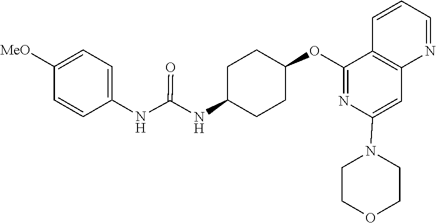 [SMILES: COc1ccc(NC(=O)N[C@H]2CC[C@@H](Oc3nc(N4CCOCC4)cc4ncccc34)CC2)cc1]

Prepared according to general procedure S starting with 1-isocyanato-4-methoxybenzene and intermediate 7 to afford the titled compound in 67% yield. ¹H NMR (400 MHz, DMSO-d₆) δ 8.80 (dd, J=4.3, 1.8 Hz, 1H), 8.31 (dd, J=8.2, 1.8 Hz, 1H), 8.10 (s, 1H), 7.33-7.25 (m, 2H), 7.21 (dd, J=8.2, 4.3 Hz, 1H), 6.86-6.77 (m, 2H), 6.54 (s, 1H), 6.18 (d, J=7.8 Hz, 1H), 5.34 (s, 1H), 3.75 (t, J=4.8 Hz, 4H), 3.69 (s, 3H), 3.66 (s, 1H), 3.50 (t, J=4.9 Hz, 4H), 2.01-1.95 (m, 2H), 1.93-1.72 (m, 4H), 1.70-1.58 (m, 2H). ¹³C NMR (100 MHz, DMSO-d₆) δ 158.81, 156.92, 155.73, 155.33, 155.19, 154.31, 134.16, 132.47, 119.69, 118.35, 114.37, 108.95, 92.47, 71.22, 66.35, 55.61, 46.65, 45.84, 28.62, 28.26. m/z calcd. for C₂₆H₃₁N₅O₄=477.2. Found [M+H]⁺=478.3.

### 1,1-Dimethyl-3-((1s,4s)-4-((7-morpholino-1,6-naphthyridin-5-yl)oxy)cyclohexyl)urea (153)

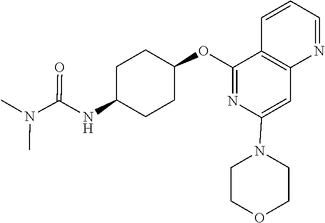 [SMILES: CN(C)C(=O)N[C@H]1CC[C@@H](Oc2nc(N3CCOCC3)cc3ncccc23)CC1]

Prepared according to general procedure Q starting with dimethylcarbamic chloride and intermediate 7 to afford the titled compound in 64% yield. ¹H NMR (400 MHz, d₆-DMSO) σ=¹H NMR (400 MHz, DMSO-d₆) δ 8.80 (dd, J=4.3, 1.9 Hz, 1H), 8.35 (dd, J=8.3, 1.9 Hz, 1H), 7.22 (dd, J=8.2, 4.4 Hz, 1H), 6.52 (s, 1H), 5.99 (d, J=7.8 Hz, 1H), 5.38 (s, 1H), 3.74 (t, J=4.8 Hz, 4H), 3.57 (s, 1H), 3.50 (t, J=4.9 Hz, 4H), 2.79 (s, 6H), 2.10-2.04 (m, 2H), 1.75-1.62 (m, 6H). ¹³C NMR (100 MHz, DMSO-d₆) δ 158.91, 158.05, 156.95, 155.72, 155.31, 132.63, 118.23, 109.00, 92.35, 70.42, 66.35, 48.84, 45.82, 36.42, 28.99, 28.27. m/z calcd. for C₂₁H₂₉N₅O₃=399.2. Found [M+H]⁺=400.3.

### 15 1-((1s,4s)-4-((7-morpholino-1,6-naphthyridin-5-yl)oxy)cyclohexyl)urea

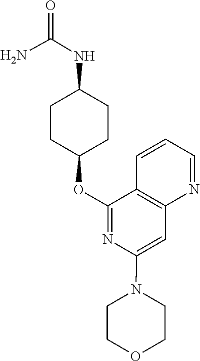 [SMILES: NC(=O)N[C@H]1CC[C@@H](Oc2nc(N3CCOCC3)cc3ncccc23)CC1]

To a mixture of CDI (1.1 eq) in THF 0.5 mL) was added intermediate 7 (1.0 eq). The reaction was stirred at RT for 1 h. 0.5M NH₃ in dioxane (8.0 eq) was then added and the reaction was stirred at RT overnight. Purification via prep HPLC gave the titled compound (yield 45%). ¹H NMR (400 MHz, DMSO) δ 8.88 (dd, J=5.4, 1.7 Hz, 1H), 8.70 (d, J=8.1 Hz, 1H), 7.39 (dd, J=8.0, 5.4 Hz, 1H), 6.50 (s, 1H), 6.05 (s, 1H), 5.37 (s, 1H), 3.78-3.71 (m, 4H), 3.67-3.60 (m, 4H), 3.53 (s, 1H), 2.05-1.95 (m, 2H), 1.85-1.66 (m, 4H), 1.65-1.51 (m, 2H). ¹³C NMR (101 MHz, DMSO) δ 159.59, 158.51, 158.33, 149.42, 148.88, 140.23, 116.82, 110.85, 84.67, 72.72, 66.22, 46.78, 45.46, 28.55, 28.17. m/z calcd. for C₁₉H₂₅N₅O₃=371.2. Found [M+H]⁺=372.2.

### 16 1-((1s,4s)-4-((7-morpholino-1,6-naphthyridin-5-yl)oxy)cyclohexyl)guanidine

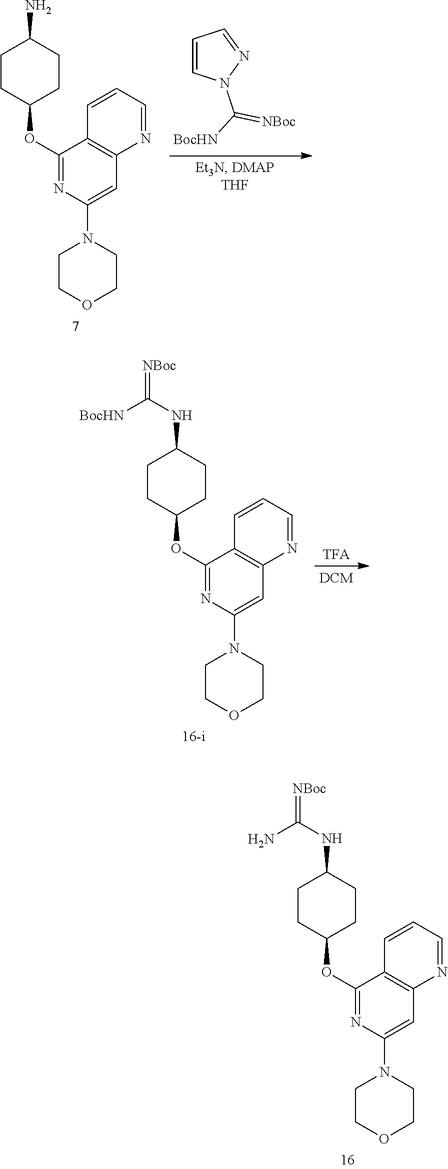

A mixture of intermediate 7 (1.0 eq), Et₃N (4.4 eq), DMAP (1.0 eq) and tert-butyl (Z)-(((tert-butoxycarbonyl)amino)(1H-pyrazol-1-yl)methylene)carbamate (2.0 eq) in THF (0.05M) was stirred at RT overnight. The reaction was partitioned between water and EtOAc. The organic layer was dried over Na₂SO₄ and concentrated to give 16-i which was used in the next step without further purification. To a solution of 16-i (1.0 eq) in DCM:TFA (1:1, 0.02M) and the reaction was stirred at RT for 1 h. Purification via prep HPLC gave the titled compound (yield 27%). ¹H NMR (400 MHz, DMSO) δ 8.88 k (dd, J=5.3, 1.7 Hz, 1H), 8.66 (d, J=8.1 Hz, 1H), 7.81 (d, J=8.3 Hz, 1H), 7.35 (dd, J=8.0, 5.2 Hz, 1H), 7.18 (broad s, 4H), 6.54 (s, 1H), 5.44 (s, 1H), 3.78-3.71 (m, 4H), 3.66-3.53 (m, 5H), 2.11-2.03 (m, 2H), 1.87-1.60 (m, 6H). ¹³C NMR (101 MHz, DMSO) δ 159.35, 158.09, 156.37, 150.41, 149.95, 139.00, 118.46, 117.01, 115.52, 110.54, 85.99, 71.36, 66.24, 48.80, 45.53, 27.98, 27.48. m/z calcd. for C₁₉H₂₆N₆O₂=370.2. Found [M+H]⁺=371.2.

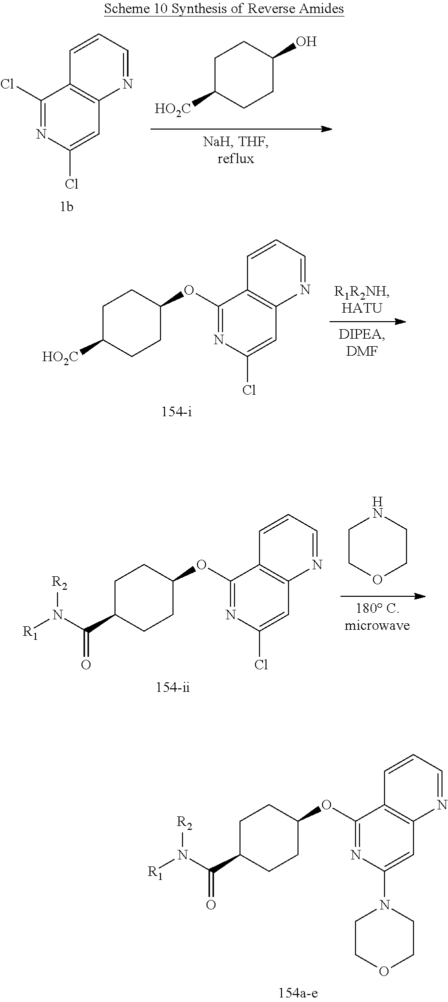

Cis-4-hydroxycyclohexanecarboxylic acid (0.98 eq, 5 mmol scale) was dissolved in THF (0.2 M), cooled to 0° C. and treated with NaH (60% dispersion in oil, 2.0 eq). After 10 minutes the mixture was warmed to room temperature and treated with 5,7-dichloro-1,6-napthyridine (1.0 eq) and then heated to reflux. After 8 h, and additional aliquot of NaH (2.0 eq) was added and reflux was continued for 18 h. After this, the mixture was cooled and water was added. The aqueous layer was extracted with EtOAc (3×), and the organic layers were discarded. The remaining aqueous layer was acidified with 1 M citric acid and it was extracted with EtOAc (3×). The combined organic layers were washed with brine, dried over Na₂SO₄, filtered, and concentrated to give crude 154-i that was used without further purification.

154-i (1.0 eq, 0.2 mmol scale) was dissolved in DMF (0.1 M) and treated with DIPEA (3.0 eq), HATU (1.5 eq) and amine (2.0 eq). The reaction was stirred at room temperature for 18 h, after which it was diluted with saturated aqueous NH₄Cl. This aqueous mixture was extracted with EtOAc (3×). The combined organic layers were washed with brine, dried over Na₂SO₄, filtered, and concentrated to give crude 154-ii that was used without further purification.

154-ii was dissolved in morpholine (0.1 M). This mixture was heated on a microwave reactor for 1 h at 180° C. Morpholine was evaporated and the mixture was purified by preparative HPLC (ACN/H₂O+TFA). Collection of pure fractions and removal of solvent by lyophilization afforded pure products as TFA salts.

### (1S,4S)-4-((7-morpholino-1,6-naphthyridin-5-yl)oxy)-N-(tetrahydro-2H-pyran-4-yl)cyclohexane-1-carboxamide/154b

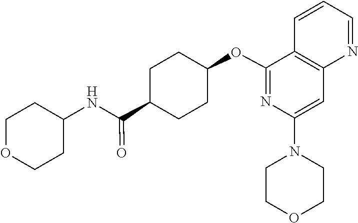 [SMILES: O=C(NC1CCOCC1)[C@H]1CC[C@@H](Oc2nc(N3CCOCC3)cc3ncccc23)CC1]

Prepared according to Scheme 10 starting with the appropriate amine to afford the titled compound. ¹H NMR (400 MHz, DMSO-d6) δ 8.88 (dd, J=5.5, 1.6 Hz, 1H), 8.71 (dd, J=8.0, 1.6 Hz, 1H), 7.75 (d, J=7.6 Hz, 1H), 7.41 (dd, J=8.0, 5.4 Hz, 1H), 6.49 (s, 1H), 5.45 (bs, 1H), 3.82 (dt, J=11.5, 3.6 Hz, 2H), 3.75 (t, J=4.9 Hz, 4H), 3.64 (t, J=4.8 Hz, 4H), 3.34 (td, J=11.5, 2.3 Hz, 2H), 2.32-2.21 (m, 1H), 2.16-2.06 (m, 2H), 1.89-1.76 (m, 2H), 1.77-1.57 (m, 6H), 1.45-1.32 (m, 2H). ¹³C NMR (101 MHz, DMSO-d₆) δ 174.45, 159.63, 158.34, 149.34, 148.81, 140.35, 116.93, 110.89, 84.58, 72.31, 66.37, 66.21, 45.44, 45.07, 42.98, 32.98, 28.84, 24.42.

### (1S,4S)-4-((7-morpholino-1,6-naphthyridin-5-yl)oxy)-N-(pyridin-2-yl)cyclohexane-1-carboxamide/154c

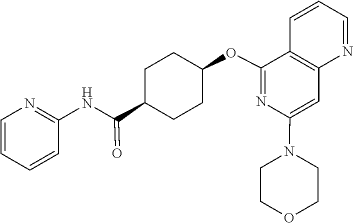 [SMILES: O=C(Nc1ccccn1)[C@H]1CC[C@@H](Oc2nc(N3CCOCC3)cc3ncccc23)CC1]

Prepared according to Scheme 10 starting with the appropriate amine to afford the titled compound. ¹H NMR (400 MHz, DMSO-d6) δ 10.50 (s, 1H), 8.89 (dd, J=5.6, 1.7 Hz, 1H), 8.79 (d, J=8.0 Hz, 1H), 8.32 (d, J=5.0 Hz, 1H), 8.08 (d, J=8.4 Hz, 1H), 7.84-7.75 (m, 1H), 7.44 (dd, J=8.0, 5.6 Hz, 1H), 7.13-7.07 (m, 1H), 6.49 (s, 1H), 5.50 (bs, 1H), 3.78-3.71 (m, 4H), 3.69-3.63 (m, 4H), 2.74-2.63 (m, 1H), 2.21-2.13 (m, 2H), 1.93-1.84 (m, 2H), 1.81-1.69 (m, 4H). ¹³C NMR (101 MHz, DMSO-d₆) δ 175.36, 159.71, 158.89, 158.53, 152.50, 148.62, 147.97, 141.34, 138.91, 119.72, 116.78, 114.01, 111.13, 83.66, 72.36, 66.21, 45.41, 43.47, 28.73, 24.17.

### (1S,4S)-4-((7-morpholino-1,6-naphthyridin-5-yl)oxy)-N-(pyridin-4-yl)cyclohexane-1-carboxamide/154e

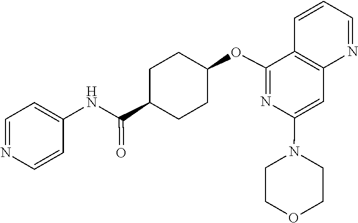 [SMILES: O=C(Nc1ccncc1)[C@H]1CC[C@@H](Oc2nc(N3CCOCC3)cc3ncccc23)CC1]

Prepared according to Scheme 10 starting with the appropriate amine to afford the titled compound. ¹H NMR (400 MHz, DMSO-d6) δ 11.34 (s, 1H), 8.87 (dd, J=5.1, 1.7 Hz, 1H), 8.70 (d, J=7.1 Hz, 2H), 8.63 (d, J=8.2 Hz, 1H), 8.07 (d, J=7.0 Hz, 2H), 7.37 (dd, J=8.1, 5.1 Hz, 1H), 6.52 (s, 1H), 5.51 (bs, 1H), 3.78-3.73 (m, 4H), 3.67-3.60 (m, 4H), 2.72-2.65 (m, 1H), 2.21-2.12 (m, 2H), 1.96-1.72 (m, 6H).

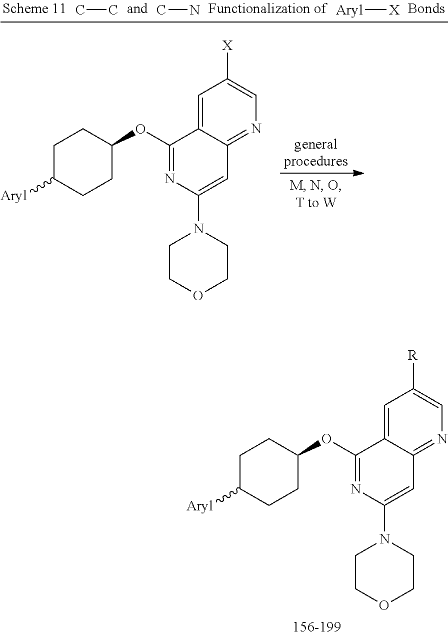

### 156 4-(5-(((1s,4s)-4-(1H-imidazol-1-yl)cyclohexyl)oxy)-3-phenyl-1,6-naphthyridin-7-yl)morpholine

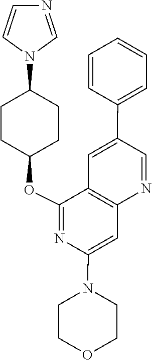 [SMILES: c1ccc(-c2cnc3cc(N4CCOCC4)nc(O[C@H]4CC[C@@H](n5ccnc5)CC4)c3c2)cc1]

Prepared according to general procedure T starting with phenylboronic acid and aryl bromide 10 to afford the titled compound. ¹H NMR (400 MHz, DMSO) δ 9.27 (s, 1H), 9.19 (d, J=2.4 Hz, 1H), 8.69 (d, J=2.3 Hz, 1H), 7.98 (s, 1H), 7.83 (d, J=1.9 Hz, 1H), 7.75 (s, 1H), 7.55 (t, J=7.6 Hz, 2H), 7.45 (t, J=7.3 Hz, 1H), 6.61 (s, 1H), 5.54-5.48 (m, 1H), 4.56-4.45 (m, 1H), 3.81-3.71 (m, 4H), 3.63-3.56 (m, 4H), 2.38-2.15 (m, 4H), 2.12-2.04 (m, 2H), 2.02-1.81 (m, 2H). ¹³C NMR (101 MHz, DMSO) δ 159.32, 157.23, 152.27, 136.91, 134.56, 132.08, 129.69, 129.62, 128.43, 127.32, 121.18, 120.40, 109.29, 89.89, 70.11, 66.31, 58.03, 45.72, 28.50, 27.84. m/z calcd. for C₂₇H₂₉N₅O₂=455.2. Found [M+H]⁺=456.4.

### 157 4-(5-(((1s,4s)-4-(1H-imidazol-1-yl)cyclohexyl)oxy)-3-(3,5-dimethylisoxazol-4-yl)-1,6-naphthyridin-7-yl)morpholine

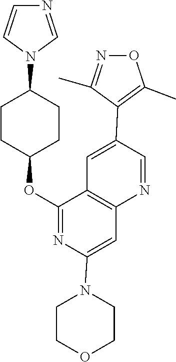 [SMILES: Cc1noc(C)c1-c1cnc2cc(N3CCOCC3)nc(O[C@H]3CC[C@@H](n4ccnc4)CC3)c2c1]

Prepared according to general procedure T starting with (3,5-dimethylisoxazol-4-yl)boronic acid and aryl bromide 10 to afford the titled compound. ¹H NMR (400 MHz, DMSO) δ 9.24 (s, 1H), 8.87 (d, J=2.3 Hz, 1H), 8.52 (d, J=2.3 Hz, 1H), 7.96 (s, 1H), 7.77 (s, 1H), 6.61 (s, 1H), 5.57 (s, 1H), 4.56-4.44 (m, 1H), 3.77-3.76 (m, 4H), 3.61-3.54 (m, 4H), 2.46 (s, 3H), 2.33-2.13 (m, 7H), 2.09-2.02 (m, 2H), 1.95-1.84 (m, 2H). ¹³C NMR (101 MHz, DMSO) δ 166.64, 159.01, 158.94, 157.36, 154.08, 153.06, 134.48, 134.08, 121.04, 120.45, 119.85, 113.30, 109.08, 90.71, 69.44, 66.29, 57.94, 45.82, 28.46, 27.73, 11.82, 10.85. m/z calcd. for C₂₆H₃₀N₆O₃=474.2. Found [M+H]⁺=475.3.

### 158 4-(5-(((1s,4s)-4-(1H-imidazol-1-yl)cyclohexyl)oxy)-3-(1-methyl-1H-pyrazol-4-yl)-1,6-naphthyridin-7-yl)morpholine

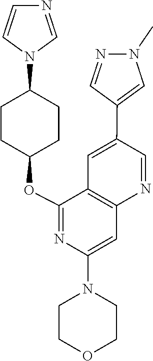 [SMILES: Cn1cc(-c2cnc3cc(N4CCOCC4)nc(O[C@H]4CC[C@@H](n5ccnc5)CC4)c3c2)cn1]

Prepared according to general procedure T starting with (1-methyl-1H-pyrazol-4-yl)boronic acid and aryl bromide 10 to afford the titled compound. ¹H NMR (400 MHz, DMSO) δ 9.28 (s, 1H), 9.15 (d, J=2.3 Hz, 1H), 8.60 (d, J=2.2 Hz, 1H), 8.38 (s, 1H), 8.12 (s, 1H), 7.99 (s, 1H), 7.77 (s, 1H), 6.58 (s, 1H), 5.56-5.49 (m, 1H), 4.59-4.47 (m, 1H), 3.92 (s, 3H), 3.80-3.69 (m, 4H), 3.61-3.54 (m, 4H), 2.33-2.19 (m, 4H), 2.13-2.04 (m, 2H), 1.97-1.85 (m, 2H). ¹³C NMR (101 MHz, DMSO) δ 158.96, 156.92, 150.39, 149.90, 136.97, 134.55, 130.72, 128.90, 122.88, 121.18, 120.42, 118.24, 109.91, 88.69, 70.10, 66.29, 57.99, 45.67, 28.45, 27.80. m/z calcd. for C₂₅H₂₉N₇O₂=459.2. Found [M+H]⁺=460.4.

### 159 4-(5-(((1s,4s)-4-(1H-imidazol-1-yl)cyclohexyl)oxy)-7-morpholino-1,6-naphthyridin-3-yl)-2-methylbut-3-yn-2-ol

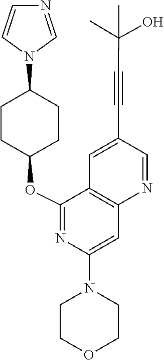 [SMILES: CC(C)(O)C#Cc1cnc2cc(N3CCOCC3)nc(O[C@H]3CC[C@@H](n4ccnc4)CC3)c2c1]

A mixture of aryl bromide 10 (1.0 eq), 2-methylbut-3-yn-2-ol (2.0 eq), Pd(PPh₃)₂Cl₂ (0.2 eq), CuI (0.2 eq), and DIEA (5.0 eq) in DMF (0.13M) was heated at 100° C. for 20 h. Purification via prep HPLC gave the titled compound (yield 26%). ¹H NMR (400 MHz, DMSO) δ 9.28 (s, 1H), 8.74 (d, J=2.2 Hz, 1H), 8.38 (d, J=2.2 Hz, 1H), 8.00 (t, J=1.8 Hz, 1H), 7.76 (s, 1H), 6.56 (s, 1H), 5.49-5.43 (m, 1H), 4.54-4.42 (m, 1H), 3.78-3.71 (m, 4H), 3.59-3.52 (m, 4H), 2.35-2.12 (m, 4H), 2.12-2.00 (m, 2H), 1.96-1.82 (m, 2H), 1.51 (s, 6H). ¹³C NMR (101 MHz, DMSO) δ 158.72, 157.24, 156.63, 153.85, 135.01, 134.56, 121.25, 120.35, 112.86, 107.86, 98.79, 92.11, 78.51, 69.92, 66.30, 64.27, 58.08, 45.65, 32.05, 28.52, 27.81. m z calcd. for C₂₆H₃₁N₅O₃=461.2. Found [M+H]⁺=462.4.

### 163 ethyl 5-(((1s,4s)-4-(1H-imidazol-1-yl)cyclohexyl)oxy)-7-morpholino-1,6-naphthyridine-3-carboxylate

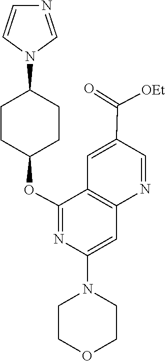 [SMILES: CCOC(=O)c1cnc2cc(N3CCOCC3)nc(O[C@H]3CC[C@@H](n4ccnc4)CC3)c2c1]

Prepared according to general procedure U starting with 10 to afford the titled compound in 46% yield. ¹H NMR (400 MHz, DMSO) δ 9.28 (s, 1H), 9.18 (d, J=2.2 Hz, 1H), 8.81 (d, J=2.1 Hz, 1H), 7.95 (s, 1H), 7.77 (s, 1H), 6.64 (s, 1H), 5.50-5.43 (m, 1H), 4.58-4.44 (m, 1H), 4.36 (q, J=7.1 Hz, 2H), 3.78-3.71 (m, 4H), 3.67-3.60 (m, 4H), 2.34-2.25 (m, 2H), 2.19-2.08 (m, 4H), 2.01-1.85 (m, 2H), 1.35 (t, J=7.1 Hz, 3H). m/z calcd. for C₂₄H₂₉N₅O₄=451.2. Found [M+H]⁺=452.4.

### 164 methyl 5-(((1s,4s)-4-(1H-imidazol-1-yl)cyclohexyl)oxy)-7-morpholino-1,6-naphthyridine-3-carboxylate

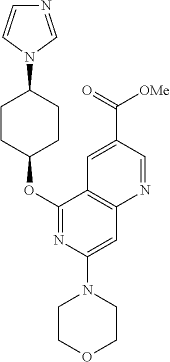 [SMILES: COC(=O)c1cnc2cc(N3CCOCC3)nc(O[C@H]3CC[C@@H](n4ccnc4)CC3)c2c1]

A mixture of 163 (1.0 eq) and LiOH monohydrate (6.0 eq) in MeOH (0.01M) was stirred at RT for 1 h. Purification via prep HPLC gave pure the titled compound. ¹H NMR (400 MHz, DMSO) δ 9.28 (s, 1H), 9.18 (d, J=2.2 Hz, 1H), 8.81 (d, J=2.2 Hz, 1H), 7.97 (s, 1H), 7.77 (s, 1H), 6.64 (s, 1H), 5.49-5.42 (m, 1H), 4.57-4.45 (m, 1H), 3.78-3.68 (m, 4H), 3.67-3.60 (m, 4H), 2.36-2.27 (m, 2H), 2.19-2.06 (m, 4H), 1.99-1.85 (m, 2H). ¹³C NMR (101 MHz, DMSO) δ 165.49, 160.02, 158.41, 156.74, 154.32, 135.42, 134.56, 121.09, 120.46, 118.62, 107.31, 91.77, 70.53, 66.30, 57.83, 52.69, 45.48, 28.36, 27.81. m/z calcd. for C₂₃H₂₇N₅O₄=437.2. Found [M+H]⁺=438.4.

### 165-(((1s,4s)-4-(1H-imidazol-1-yl)cyclohexyl)oxy)-7-morpholino-1,6-naphthyridine-3-carboxylic acid

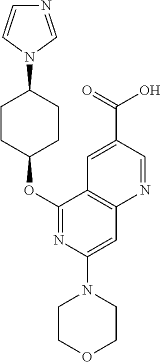 [SMILES: O=C(O)c1cnc2cc(N3CCOCC3)nc(O[C@H]3CC[C@@H](n4ccnc4)CC3)c2c1]

A mixture of 163 (1.0 eq) and LiOH monohydrate (6.0 eq) in MeOH:water (1:1, 0.02M) was stirred at RT for 1 h. Purification via prep HPLC gave the titled compound (yield 52%). ¹H NMR (400 MHz, DMSO) δ 9.28 (s, 1H), 9.18 (d, J=2.2 Hz, 1H), 8.81 (d, J=2.2 Hz, 1H), 7.96 (s, 1H), 7.75 (s, 1H), 6.63 (s, 1H), 5.48-5.42 (m, 1H), 4.56-4.43 (m, 1H), 3.79-3.72 (m, 4H), 3.66-3.59 (m, 4H), 2.36-2.28 (m, 2H), 2.23-2.05 (m, 4H), 2.01-1.84 (m, 2H). ¹³C NMR (101 MHz, DMSO) δ 166.47, 160.00, 158.29, 156.49, 154.63, 135.58, 134.55, 121.11, 120.43, 119.73, 107.46, 91.62, 70.48, 66.30, 57.87, 45.51, 28.40, 27.81. m/z calcd. for C₂₂H₂₅N₅O₄=423.2. Found [M+H]⁺=424.2.

### 166 5-(((1s,4s)-4-(1H-imidazol-1-yl)cyclohexyl)oxy)-7-morpholino-1,6-naphthyridine-3-carboxamide

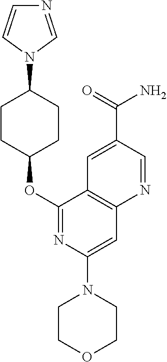 [SMILES: NC(=O)c1cnc2cc(N3CCOCC3)nc(O[C@H]3CC[C@@H](n4ccnc4)CC3)c2c1]

Prepared according to general procedure 0 starting with 165 and NH₄Cl to afford the titled compound in 45% yield. ¹H NMR (400 MHz, DMSO) δ 9.25 (s, 1H), 9.21 (s, 1H), 8.84 (s, 1H), 8.25 (s, 1H), 7.95 (s, 1H), 7.77 (s, 1H), 7.61 (s, 1H), 6.60 (s, 1H), 5.50 (s, 1H), 4.56-4.46 (m, 1H), 3.79-3.72 (m, 4H), 2.36-2.04 (m, 6H), 1.99-1.85 (m, 2H). ¹³C NMR (101 MHz, DMSO) δ 166.29, 159.84, 158.01, 153.21, 134.53, 133.96, 123.07, 121.04, 120.48, 107.78, 90.73, 70.19, 66.31, 57.90, 45.56, 28.40, 27.72. m/z calcd. for C₂₂H₂₆N₆O₃=422.2. Found [M+H]⁺=423.3.

### 167 5-(((1s,4s)-4-(1H-imidazol-1-yl)cyclohexyl)oxy)-N-acetyl-7-morpholino-1,6-naphthyridine-3-carboxamide

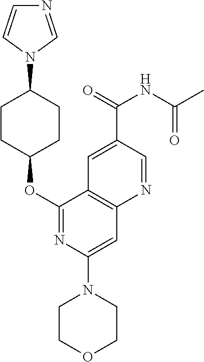 [SMILES: CC(=O)NC(=O)c1cnc2cc(N3CCOCC3)nc(O[C@H]3CC[C@@H](n4ccnc4)CC3)c2c1]

A mixture of 166 (1.0 eq acetic anhydride (1.3 eq), DIEA (2.2 eq) and DMAP (cat.) in ACN (0.03M) was heated via microwave at 120° C. for 1 h then at 130° C. for 1 h. Another portion of acetic anhydride (1.3 eq) and DIEA (2.2 eq) was added and the reaction mixture was heated at 130° C. for 1 h. Purification via prep HPLC gave the titled compound (yield 25%). ¹H NMR (400 MHz, DMSO) δ 11.24 (s, 1H), 9.26 (s, 1H), 9.15 (d, J=2.3 Hz, 1H), 8.85 (d, J=2.3 Hz, 1H), 7.96 (s, 1H), 7.76 (s, 1H), 6.63 (s, 1H), 5.55-5.49 (m, 1H), 4.58-4.45 (m, 1H), 3.79-3.72 (m, 4H), 3.67-3.60 (m, 4H), 2.37 (s, 3H), 2.32-2.14 (m, 4H), 2.14-2.06 (m, 2H), 1.97-1.87 (m, 2H). ¹³C NMR (101 MHz, DMSO) δ 172.61, 165.47, 159.98, 158.33, 156.27, 154.06, 135.05, 134.55, 122.15, 121.03, 120.47, 107.10, 91.54, 70.09, 66.30, 57.85, 45.49, 28.39, 27.73, 26.03. m/z calcd. for C₂₄H₂₈N₆O₄=464.2. Found [M+H]⁺=465.2.

### 168 5-(((1s,4s)-4-(1H-imidazol-1-yl)cyclohexyl)oxy)-N-ethyl-7-morpholino-1,6-naphthyridine-3-carboxamide

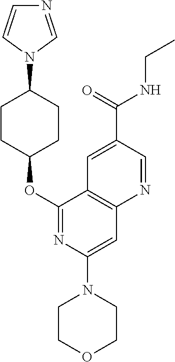 [SMILES: CCNC(=O)c1cnc2cc(N3CCOCC3)nc(O[C@H]3CC[C@@H](n4ccnc4)CC3)c2c1]

Prepared according to general procedure 0 starting with 165 and 70% diethylamine in H₂O to afford the titled compound in 7% yield. ¹H NMR (400 MHz, DMSO) δ 9.25 (s, 1H), 9.17 (d, J=2.3 Hz, 1H), 8.76 (d, J=2.3 Hz, 1H), 8.71 (t, J=5.5 Hz, 1H), 7.95 (s, 1H), 7.76 (s, 1H), 6.61 (s, 1H), 5.50 (s, 1H), 4.56-4.43 (m, 1H), 3.79-3.72 (m, 4H), 3.59-3.56 (m, 4H), 2.36-2.06 (m, 6H), 1.99-1.84 (m, 2H), 1.16 (t, J=7.2 Hz, 3H). m/z calcd. for C₂₄H₃₀N₆O₃=450.2. Found [M+H]⁺=451.3.

### 169 5-(((1s,4s)-4-(1H-imidazol-1-yl)cyclohexyl)oxy)-7-morpholino-N-propyl-1,6-naphthyridine-3-carboxamide

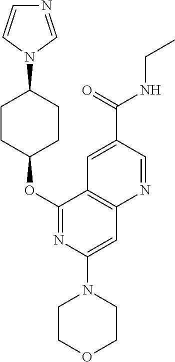 [SMILES: CCNC(=O)c1cnc2cc(N3CCOCC3)nc(O[C@H]3CC[C@@H](n4ccnc4)CC3)c2c1]

Prepared according to general procedure 0 starting with 165 and propylamine to afford the titled compound in 29% yield. ¹H NMR (400 MHz, DMSO) δ 9.26 (s, 1H), 9.18 (d, J=2.3 Hz, 1H), 8.79 (d, J=2.3 Hz, 1H), 8.73 (t, J=5.6 Hz, 1H), 7.95 (s, 1H), 7.76 (s, 1H), 6.61 (s, 1H), 5.51 (s, 1H), 4.58-4.47 (m, 1H), 3.79-3.72 (m, 4H), 3.64-3.57 (m, 4H), 3.27 (q, J=6.7 Hz, 2H), 2.33-2.05 (m, 6H), 1.97-1.86 (m, 2H), 1.64-1.48 (m, 2H), 0.92 (t, J=7.4 Hz, 3H). m/z calcd. for C₂₅H₃₂N₆O₃=464.3. Found [M+H]⁺=465.4.

### 170 5-(((1s,4s)-4-(1H-imidazol-1-yl)cyclohexyl)oxy)-N,N-dimethyl-7-morpholino-1,6-naphthyridine-3-carboxamide

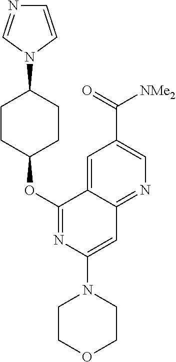 [SMILES: CN(C)C(=O)c1cnc2cc(N3CCOCC3)nc(O[C@H]3CC[C@@H](n4ccnc4)CC3)c2c1]

Prepared according to general procedure 0 starting with 165 and dimethylamine hydrochloride to afford the titled compound in 35% yield. ¹H NMR (400 MHz, DMSO) δ 9.27 (d, J=1.5 Hz, 1H), 8.85 (d, J=2.2 Hz, 1H), 8.53 (d, J=2.2 Hz, 1H), 7.98 (t, J=1.7 Hz, 1H), 7.76 (t, J=1.7 Hz, 1H), 6.59 (s, 1H), 5.54-5.48 (m, 1H), 4.55-4.43 (m, 1H), 3.79-3.72 (m, 4H), 3.62-3.55 (m, 4H), 3.03 (s, 6H), 2.32-2.02 (m, 6H), 1.98-1.82 (m, 2H). ¹³C NMR (101 MHz, DMSO) δ 167.93, 159.38, 157.68, 154.47, 152.83, 134.54, 133.21, 125.70, 121.14, 120.41, 107.80, 91.13, 69.85, 66.30, 57.98, 45.67, 28.46, 27.77. m/z calcd. for C₂₄H₃₀N₆O₃=450.2. Found [M+H]⁺=451.2.

### 171 5-(((1s,4s)-4-(1H-imidazol-1-yl)cyclohexyl)oxy)-N-methoxy-7-morpholino-1,6-naphthyridine-3-carboxamide

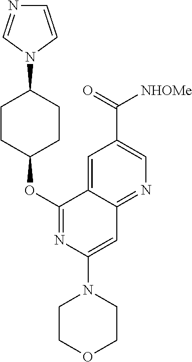

Prepared according to general procedure V starting with 165 and 0-methylhydroxylamine hydrochloride to afford the titled compound in 64% yield. ¹H NMR (400 MHz, MeOD) δ 8.96 (d, J=7.9, Hz, 1H), 8.77-8.70 (m, 2H), 7.42 (dd, J=7.9, 5.8 Hz, 1H), 6.46 (s, 1H), 5.69-5.61 (m, 1H), 5.09 (tt, J=10.8, 4.0 Hz, 1H), 3.89-3.77 (m, 8H), 2.59-2.44 (m, 2H), 2.43-2.22 (m, 4H), 2.17-2.03 (m, 2H). ¹³C NMR (101 MHz, DMSO) δ 159.75, 158.06, 155.30, 152.51, 134.55, 133.54, 121.10, 121.05, 120.48, 107.65, 90.87, 70.24, 66.30, 63.94, 57.85, 45.53, 28.38, 27.73. m/z calcd. for C₂₃H₂₈N₆O₄=452.2. Found [M+H]⁺=453.2.

### 172 5-(((1s,4s)-4-(1H-imidazol-1-yl)cyclohexyl)oxy)-N-hydroxy-7-morpholino-1,6-naphthyridine-3-carboxamide

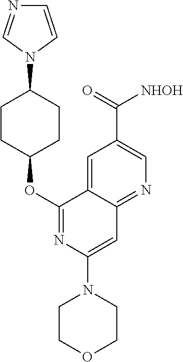

Prepared according to general procedure V starting with 165 and hydroxylamine hydrochloride to afford the titled compound in 50% yield. ¹H NMR (400 MHz, DMSO) δ 14.49 (broad s, 1H), 11.47 (s, 1H), 9.26 (d, J=1.6 Hz, 1H), 9.10 (d, J=2.3 Hz, 1H), 8.74 (d, J=2.3 Hz, 1H), 7.95 (t, J=1.7 Hz, 1H), 7.77 (t, J=1.7 Hz, 1H), 6.60 (s, 1H), 5.52-5.43 (m, 1H), 4.57-4.44 (m, 1H), 3.79-3.72 (m, 4H), 3.64-3.57 (m, 4H), 2.37-2.05 (m, 6H), 1.97-1.86 (m, 2H). ¹³C NMR (101 MHz, DMSO) δ 159.69, 157.95, 155.22, 152.65, 134.54, 132.97, 121.67, 121.02, 120.50, 107.67, 90.93, 70.13, 66.29, 57.88, 45.56, 28.39, 27.70. m/z calcd. for C₂₂H₂₆N₆O₄=438.2. Found [M+H]⁺=439.2.

### 173 5-(((1s,4s)-4-(1H-imidazol-1-yl)cyclohexyl)oxy)-7-morpholino-N-(propylsulfonyl)-1,6-naphthyridine-3-carboxamide

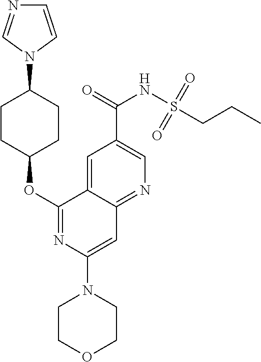 [SMILES: CCCS(=O)(=O)NC(=O)c1cnc2cc(N3CCOCC3)nc(O[C@H]3CC[C@@H](n4ccnc4)CC3)c2c1]

Prepared according to general procedure W starting with 165 and propane-1-sulfonamide to afford the titled compound in 8% yield. ¹H NMR (400 MHz, DMSO) δ 9.27 (d, J=1.5 Hz, 1H), 9.16 (d, J=2.3 Hz, 1H), 8.86 (d, J=2.3 Hz, 1H), 7.97 (t, J=1.8 Hz, 1H), 7.76 (t, J=1.7 Hz, 1H), 6.63 (s, 1H), 5.51-5.45 (m, 1H), 4.56-4.46 (m, 1H), 3.78-3.71 (m, 4H), 3.67-3.61 (m, 4H), 3.53 (t, 2H), 2.35-2.04 (m, 6H), 1.92 (t, J=13.3 Hz, 2H), 1.82-1.70 (m, 2H), 1.01 (t, J=7.4 Hz, 3H). ¹³C NMR (101 MHz, DMSO) δ 165.31, 160.05, 158.37, 156.87, 154.09, 134.95, 134.59, 121.00, 120.64, 120.53, 106.92, 91.91, 70.29, 66.30, 57.78, 54.66, 45.42, 28.36, 27.74, 17.17, 13.02. m/z calcd. for C₂₅H₃₂N₆O₅S=528.2. Found [M+H]⁺=529.2.

### 174 5-(((1s,4s)-4-(1H-imidazol-1-yl)cyclohexyl)oxy)-N-(cyclopropylsulfonyl)-7-morpholino-1,6-naphthyridine-3-carboxamide

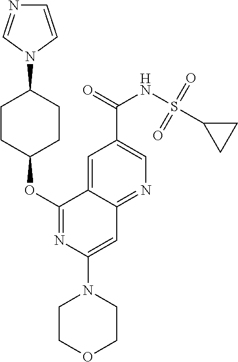 [SMILES: O=C(NS(=O)(=O)C1CC1)c1cnc2cc(N3CCOCC3)nc(O[C@H]3CC[C@@H](n4ccnc4)CC3)c2c1]

Prepared according to general procedure W starting with 165 and cyclopropanesulfonamide to afford the titled compound in 8% yield. ¹H NMR (400 MHz, DMSO) δ 9.27 (d, J=1.6 Hz, 1H), 9.17 (d, J=2.3 Hz, 1H), 8.86 (d, J=2.3 Hz, 1H), 7.97 (t, J=1.8 Hz, 1H), 7.76 (t, J=1.7 Hz, 1H), 6.64 (s, 1H), 5.48 (t, J=3.1 Hz, 1H), 4.56-4.46 (m, 1H), 3.78-3.71 (m, 4H), 3.67-3.60 (m, 4H), 3.22-3.11 (m, 1H), 2.33-2.07 (m, 6H), 1.97-1.86 (m, 2H), 1.19-1.09 (m, 4H). ¹³C NMR (101 MHz, DMSO) δ 165.03, 160.07, 158.39, 156.76, 154.01, 135.02, 134.58, 121.03, 120.60, 120.50, 106.99, 91.82, 70.33, 66.30, 57.79, 45.43, 31.50, 28.37, 27.74. m/z calcd. for C₂₅H₃₀N₆O₅S=526.2. Found [M+H]⁺=527.4.

### 175 5-(((1s,4s)-4-(1H-imidazol-1-yl)cyclohexyl)oxy)-N-(methylsulfonyl)-7-morpholino-1,6-naphthyridine-3-carboxamide

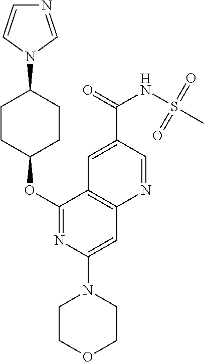 [SMILES: CS(=O)(=O)NC(=O)c1cnc2cc(N3CCOCC3)nc(O[C@H]3CC[C@@H](n4ccnc4)CC3)c2c1]

Prepared according to general procedure W starting with 165 and methanesulfonamide to afford the titled compound in 12% yield. ¹H NMR (400 MHz, DMSO) δ 9.27 (d, J=2.1 Hz, 1H), 8.84-8.78 (m, 1H), 8.14 (s, 1H), 7.96-7.85 (m, 1H), 7.36 (s, 1H), 7.06-6.93 (m, 1H), 6.59 (s, 1H), 6.52 (s, 1H), 5.51-5.42 (m, 1H), 4.35-4.23 (m, 1H), 3.79-3.72 (m, 5H), 3.59-3.51 (m, 5H), 3.04 (s, 1H), 2.92 (s, 3H), 2.33-2.17 (m, 2H), 2.17-1.96 (m, 4H), 1.98-1.81 (m, 2H). ¹³C NMR (101 MHz, DMSO) δ 168.66, 163.51, 159.61, 157.30, 156.23, 155.69, 132.35, 128.74, 107.84, 92.61, 70.05, 66.34, 55.21, 45.74, 40.97, 28.84, 28.73. m/z calcd. for C₂₃H₂₈N₆O₅S=500.2. Found [M+H]⁺=501.2.

### 178 2-((2-(((1s,4s)-4-((3-bromo-7-morpholino-1,6-naphthyridin-5-yl)oxy)cyclohexyl)amino)pyrimidin-5-yl)oxy)-N,N-dimethylacetamide

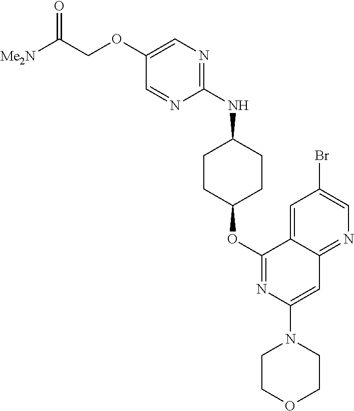 [SMILES: CN(C)C(=O)COc1cnc(N[C@H]2CC[C@@H](Oc3nc(N4CCOCC4)cc4ncc(Br)cc34)CC2)nc1]

Prepared according to general procedure H starting with 54-i and intermediate 8 to afford the titled compound in 23% yield. H NMR (400 MHz, DMSO) δ 8.85 (d, J=2.4 Hz, 1H), 8.46 (d, J=2.5 Hz, 1H), 8.12 (s, 2H), 6.97 (broad s, 1H), 6.54 (s, 1H), 5.41 (s, 1H), 4.79 (s, 2H), 3.80 (s, 1H), 3.77-3.70 (m, 4H), 3.56-3.49 (m, 4H), 2.96 (s, 3H), 2.84 (s, 3H), 2.10 (d, J=8.1 Hz, 2H), 1.80-1.74 (m, 6H). ¹³C NMR (101 MHz, DMSO) δ 167.58, 158.18, 157.71, 157.20, 155.67, 153.61, 146.71, 145.33, 134.32, 111.86, 109.69, 91.60, 71.11, 67.89, 66.30, 49.08, 45.66, 35.93, 35.38, 28.58, 27.57. m/z calcd. for C₂₆H₃₂BrN₇O₄=585.2. Found [M+H]⁺=586.2.

### 179 ethyl 5-(((1s,4s)-4-((5-(2-(dimethylamino)-2-oxoethoxy)pyrimidin-2-yl)amino)cyclohexyl)oxy)-7-morpholino-1,6-naphthyridine-3-carboxylate

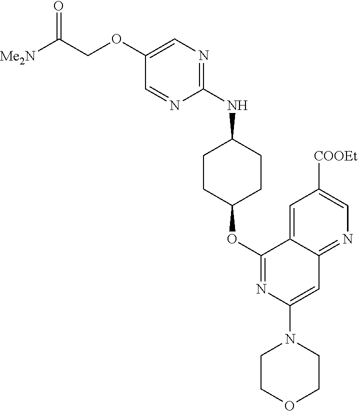 [SMILES: CCOC(=O)c1cnc2cc(N3CCOCC3)nc(O[C@H]3CC[C@@H](Nc4ncc(OCC(=O)N(C)C)cn4)CC3)c2c1]

Prepared according to general procedure U starting with 178 to afford the titled compound in 33% yield. ¹H NMR (400 MHz, DMSO) δ 9.19 (d, J=2.2 Hz, 1H), 8.79 (d, J=2.2 Hz, 1H), 8.12 (s, 2H), 7.00 (broad s, 1H), 6.60 (s, 1H), 5.39 (s, 1H), 4.79 (s, 2H), 4.39 (q, J=7.1 Hz, 2H), 3.87-3.71 (m, 5H), 3.68-3.61 (m, 4H), 2.96 (s, 3H), 2.84 (s, 3H), 2.21-2.07 (m, 2H), 1.86-1.71 (m, 6H), 1.37 (t, J=7.1 Hz, 3H). ¹³C NMR (101 MHz, DMSO) δ 167.58, 164.76, 160.19, 158.63, 157.77, 155.81, 153.73, 146.70, 145.36, 135.63, 118.59, 107.73, 90.72, 72.26, 67.87, 66.29, 61.49, 48.91, 45.48, 35.93, 35.37, 28.36, 27.73. m/z calcd. for C₂₉H₃₇N₇O₆=579.3. Found [M+H]⁺=580.2.

### 180 5-(((1s,4s)-4-((5-(2-(dimethylamino)-2-oxoethoxy)pyrimidin-2-yl)amino)cyclohexyl)oxy)-7-morpholino-1,6-naphthyridine-3-carboxylic acid

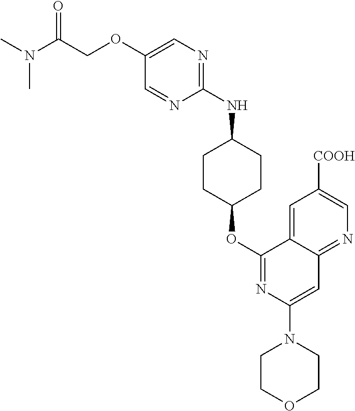 [SMILES: CN(C)C(=O)COc1cnc(N[C@H]2CC[C@@H](Oc3nc(N4CCOCC4)cc4ncc(C(=O)O)cc34)CC2)nc1]

A mixture of 179 (1.0 eq) and LiOH (4.0 eq) in THF:water (1:2, 0.02M) was stirred at RT for 2 h. The reaction was quenched with TFA and purified via prep HPLC to afford the titled compound (yield 29%). ¹H NMR (400 MHz, DMSO) δ 9.17 (d, J=2.1 Hz, 1H), 8.75 (d, J=2.2 Hz, 1H), 8.09 (s, 2H), 6.90 (d, J=7.7 Hz, 1H), 6.50-6.80 (bs, 2H), 6.58 (s, 1H), 5.38 (s, 1H), 4.77 (s, 2H), 3.74 (t, J=4.8 Hz, 5H), 3.58 (t, J=4.9 Hz, 4H), 2.96 (s, 3H), 2.84 (s, 3H), 2.15 (t, J=7.7 Hz, 2H), 1.77 (q, J=13.1, 12.7 Hz, 6H). m/z calcd. for C₂₇H₃₃N₇O₆=551.3. Found [M+H]⁺=552.2.

### 181 5-(((1s,4s)-4-((5-(carboxymethoxy)pyrimidin-2-yl)amino)cyclohexyl)oxy)-7-morpholino-1,6-naphthyridine-3-carboxylic acid

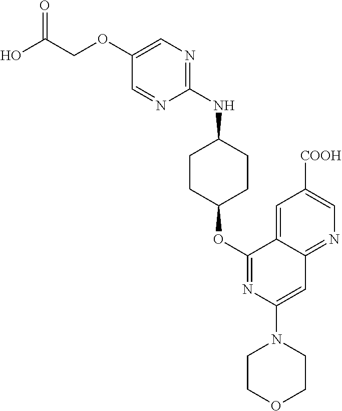 [SMILES: O=C(O)COc1cnc(N[C@H]2CC[C@@H](Oc3nc(N4CCOCC4)cc4ncc(C(=O)O)cc34)CC2)nc1]

A mixture of 179 (1.0 eq) and LiOH (6.0 eq) in MeOH:water (1:1, 0.05M) was stirred at RT for 2 h. An additional portion of LiOH (7.5 eq) was added and the reaction was stirred at RT for another 2.5 h. The reaction was concentrated and the resultant crude material was diluted with water and washed with DCM. The aqueous layer was acidified with 1M HCl aq. and extracted with EtOAc. The combined organic layers was dried over Na₂SO₄ and concentrated. During concentration, the product precipitated out and was filtered to give the title compound (yield 10%). ¹H NMR (400 MHz, DMSO) δ 12.40-13.80 (broad s, 2H), 9.17 (d, J=2.2 Hz, 1H), 8.79 (d, J=2.2 Hz, 1H), 8.12 (s, 2H), 6.97 (s, 1H), 6.59 (s, 1H), 5.39 (s, 1H), 4.64 (s, 2H), 3.82-3.78 (m, 1H), 3.78-3.66 (m, 4H), 3.66-3.59 (m, 4H), 2.18-2.11 (m, 2H), 1.85-1.70 (m, 6H)¹³C NMR (101 MHz, DMSO) δ 170.69, 166.40, 161.41, 160.10, 158.46, 158.24, 156.28, 154.44, 146.80, 145.04, 135.43, 119.58, 107.62, 91.17, 71.89, 66.85, 66.29, 49.02, 45.50, 28.50, 27.76. m/z calcd. for C₂₅H₂₈N₆O₇=524.2. Found [M+H]⁺=525.2.

### 182 N,N-dimethyl-2-((2-(((1s,4s)-4-((3-(methylsulfonamido)-7-morpholino-1,6-naphthyridin-5-yl)oxy)cyclohexyl)amino)pyrimidin-5-yl)oxy)acetamide

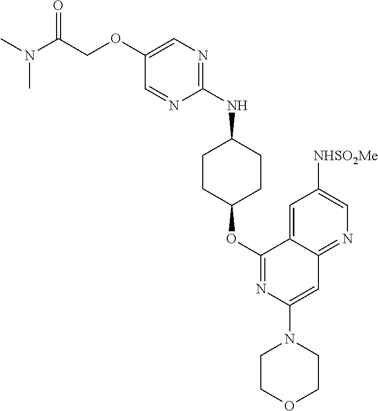

Prepared according to general procedures M and N starting with 178 and methanesulfonyl chloride to afford the titled compound in 15% yield over 2 steps. ¹H NMR (400 MHz, DMSO) δ 9.92 (s, 1H), 8.71 (d, J=2.6 Hz, 1H), 8.14 (d, J=2.6 Hz, 1H), 8.09 (s, 2H), 6.80 (d, J=7.3 Hz, 1H), 6.56 (m, 1H), 5.34 (s, 1H), 4.78 (s, 2H), 3.74 (m, 5H), 3.48 (m, 4H), 3.05 (s, 3H), 2.95 (s, 3H), 2.84 (s, 3H), 2.15-2.04 (m, 2H), 1.76 (m, Hz, 6H). ¹³C NMR (101 MHz, DMSO) δ 167.61, 158.59, 158.16, 156.50, 152.98, 150.84, 146.70, 145.41, 129.28, 123.22, 108.51, 92.37, 71.41, 67.78, 66.31, 48.96, 45.93, 35.92, 35.37, 28.47, 27.83. m/z calcd. for C₂₇H₃₆N₈O₆S=600.3. Found [M+H]⁺=601.3.

### N-((1s,4s)-4-((3-Bromo-7-morpholino-1,6-naphthyridin-5-yl)oxy)cyclohexyl)pyrimidin-2-amine (18a

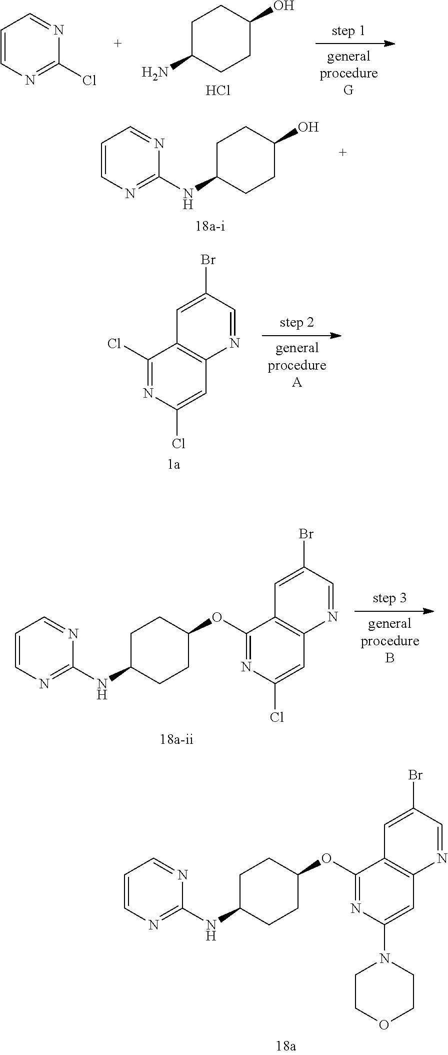

Prepared according to general procedures G, A and B, successively, starting with 2-chloropyrimidine and (1s,4s)-4-aminocyclohexan-1-ol hydrochloride to afford the titled compound in 35% yield over 3 steps. ¹H NMR (400 MHz, DMSO-d₆) δ 8.84 (d, J=2.4 Hz, 1H), 8.45 (dd, J=2.5, 0.8 Hz, 1H), 8.26 (d, J=4.7 Hz, 2H), 7.12 (d, J=8.1 Hz, 1H), 6.57-6.50 (m, 2H), 5.42 (s, 1H), 3.90 (s, 1H), 3.74 (t, J=4.9 Hz, 5H), 3.53 (t, J=4.8 Hz, 4H), 2.15-2.05 (m, 2H), 1.85-1.70 (m, 6H). m/z calcd. for C₂₂H₂₅BrN₆O₂=484.1. Found [M+H]⁺=485.3.

### 183 7-morpholino-5-(((1s,4s)-4-(pyrimidin-2-ylamino)cyclohexyl)oxy)-1,6-naphthyridine-3-carboxylic acid

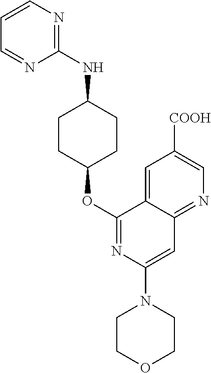 [SMILES: O=C(O)c1cnc2cc(N3CCOCC3)nc(O[C@H]3CC[C@@H](Nc4ncccn4)CC3)c2c1]

Prepared according to procedure U starting with 18a to afford the ethyl ester intermediate which was then stirred with LiOH monohydrate (6.0 eq) in MeOH:water (1:1, 0.02M) for 1 h at RT. Purification via prep HPLC gave the titled compound (yield 46% over 2 steps). ¹H NMR (400 MHz, DMSO) δ 13.32 (broad s, 1H), 9.18 (d, J=2.2 Hz, 1H), 8.81 (d, J=2.2 Hz, 1H), 8.31 (d, J=4.8 Hz, 2H), 7.49 (s, 1H), 6.63-6.56 (m, 2H), 5.42 (s, 1H), 3.89 (s, 1H), 3.78-3.71 (m, 4H), 2.20-2.13 (m, 2H), 1.86-1.73 (m, 6H). m/z calcd. for C₂₃H₂₆N₆O₄=450.2. Found [M+H]⁺=451.2.

### 184 7-morpholino-5-(((1s,4s)-4-(pyrimidin-2-ylamino)cyclohexyl)oxy)-1,6-naphthyridin-3-amine

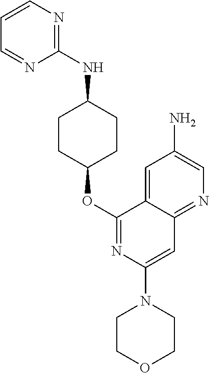 [SMILES: Nc1cnc2cc(N3CCOCC3)nc(O[C@H]3CC[C@@H](Nc4ncccn4)CC3)c2c1]

Prepared according to general procedure M starting with 18a to afford the titled compound in 49% yield. ¹H NMR (400 MHz, DMSO) δ 8.42 (d, J=2.8 Hz, 1H), 8.27 (d, J=4.7 Hz, 2H), 7.38 (d, J=2.8 Hz, 1H), 7.10 (d, J=7.5 Hz, 1H), 6.54 (t, J=4.7 Hz, 1H), 6.41 (s, 1H), 5.44 (s, 2H), 5.29 (s, 1H), 3.85 (s, 1H), 3.74 (t, J=4.8 Hz, 4H), 2.19-2.04 (m, 2H), 1.83-1.71 (m, 6H). ¹³C NMR (101 MHz, DMSO) δ 162.15, 158.38, 157.35, 153.70, 148.26, 147.30, 141.00, 110.22, 109.98, 93.55, 70.26, 66.41, 55.40, 48.74, 46.60, 28.66, 27.66.

### 186 N-(7-morpholino-5-(((1s,4s)-4-(pyrimidin-2-ylamino)cyclohexyl)oxy)-1,6-naphthyridin-3-yl)cyclopropanesulfonamide

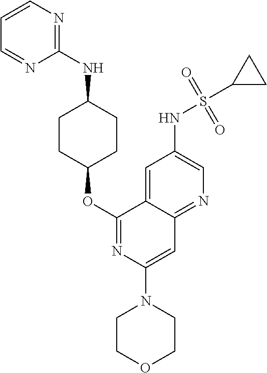 [SMILES: O=S(=O)(Nc1cnc2cc(N3CCOCC3)nc(O[C@H]3CC[C@@H](Nc4ncccn4)CC3)c2c1)C1CC1]

Prepared according to general procedure N starting with 184 and cyclopropanesulfonyl chloride to afford the titled compound in 53% yield. ¹H NMR (400 MHz, DMSO) δ 8.69 (d, J=2.6 Hz, 1H), 8.26 (d, J=4.7 Hz, 2H), 8.14 (d, J=2.6 Hz, 1H), 7.12 (d, J=7.4 Hz, 1H), 6.58-6.51 (m, 2H), 5.35 (s, 1H), 3.90-3.80 (m, 1H), 3.78-3.71 (m, 4H), 2.72-2.61 (m, 1H), 2.16-2.06 (m, 2H), 1.90-1.66 (m, 6H). ¹³C NMR (101 MHz, DMSO) δ 162.12, 158.52, 158.40, 156.35, 152.69, 151.48, 129.98, 123.61, 110.26, 108.60, 92.40, 71.16, 66.32, 48.58, 45.94, 29.92, 28.50, 27.68, 5.46. m/z calcd. for C₂₅H₃₁N₇O₄S=525.2. Found [M+H]⁺=526.2.

### 190 N-(7-morpholino-5-(((1s,4s)-4-(pyrimidin-2-ylamino)cyclohexyl)oxy)-1,6-naphthyridin-3-yl)cyclopentanesulfonamide

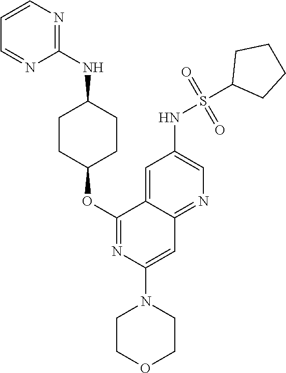 [SMILES: O=S(=O)(Nc1cnc2cc(N3CCOCC3)nc(O[C@H]3CC[C@@H](Nc4ncccn4)CC3)c2c1)C1CCCC1]

Prepared according to general procedure N starting with 184 and cyclopentanesulfonyl chloride to afford the titled compound in 8% yield. ¹H NMR (400 MHz, DMSO) δ 10.01 (s, 1H), 8.70 (d, J=2.7 Hz, 1H), 8.32 (d, J=4.8 Hz, 2H), 8.20 (d, J=2.6 Hz, 1H), 7.36 (s, 1H), 6.60 (t, J=4.8 Hz, 1H), 6.53 (s, 1H), 5.35 (s, 1H), 3.86 (s, 1H), 3.78-3.71 (m, 4H), 2.10 (q, J=5.9, 3.9 Hz, 2H), 1.97-1.48 (m, 15H).

### 191 N-(7-morpholino-5-(((1s,4s)-4-(pyrimidin-2-ylamino)cyclohexyl)oxy)-1,6-naphthyridin-3-yl)cyclobutanesulfonamide

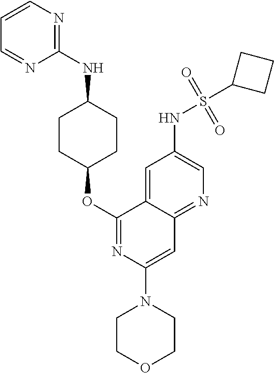 [SMILES: O=S(=O)(Nc1cnc2cc(N3CCOCC3)nc(O[C@H]3CC[C@@H](Nc4ncccn4)CC3)c2c1)C1CCC1]

Prepared according to general procedure N starting with 184 and cyclobutanesulfonyl chloride to afford the titled compound in 43% yield. ¹H NMR (400 MHz, DMSO) δ 9.99 (s, 1H), 8.67 (d, J=2.6 Hz, 1H), 8.35 (d, J=4.9 Hz, 2H), 8.19 (d, J=2.6 Hz, 1H), 7.55 (s, 1H), 6.64 (t, J=4.9 Hz, 1H), 6.52 (s, 1H), 5.36 (s, 1H), 4.00 (p, J=8.3 Hz, 1H), 3.81-3.71 (m, 4H), 3.54-3.47 (m, 4H), 2.30 (qd, J=9.2, 8.8, 2.8 Hz, 2H), 2.25-2.15 (m, 2H), 2.15-2.06 (m, 2H), 1.98-1.70 (m, 8H).

### 192 1-fluoro-N-(7-morpholino-5-(((1s,4s)-4-(pyrimidin-2-ylamino)cyclohexyl)oxy)-1,6-naphthyridin-3-yl)methanesulfonamide

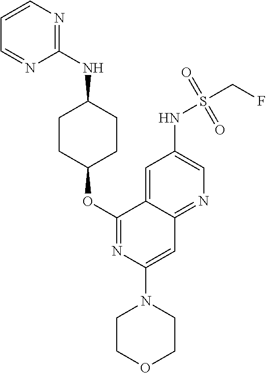 [SMILES: O=S(=O)(CF)Nc1cnc2cc(N3CCOCC3)nc(O[C@H]3CC[C@@H](Nc4ncccn4)CC3)c2c1]

Prepared according to general procedure N starting with 184 and fluoromethanesulfonyl chloride to afford the titled compound in 55% yield. ¹H NMR (400 MHz, DMSO) δ 10.68 (s, 1H), 8.69 (d, J=2.6 Hz, 1H), 8.35 (d, J=4.9 Hz, 2H), 8.19 (s, 1H), 7.56 (s, 1H), 6.65 (t, J=4.9 Hz, 1H), 6.54 (s, 1H), 5.58 (d, J=45.8 Hz, 2H), 5.37 (s, 1H), 3.87 (s, 1H), 3.74 (t, J=5.0 Hz, 4H), 3.52 (t, J=4.9 Hz, 4H), 2.15-2.06 (m, 2H), 1.87-1.68 (m, 6H). ¹³C NMR (101 MHz, DMSO) δ 159.77, 158.76, 158.20, 157.01, 151.82, 149.69, 127.60, 126.57, 110.22, 108.87, 90.58, 90.49, 88.51, 71.51, 66.27, 48.79, 45.78, 28.32, 27.44.

### 193 2,2-difluoro-N-(7-morpholino-5-(((1s,4s)-4-(pyrimidin-2-ylamino)cyclohexyl)oxy)-1,6-naphthyridin-3-yl)ethane-1-sulfonamide

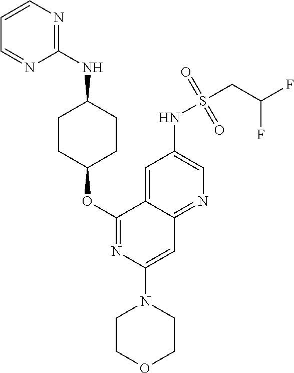 [SMILES: O=S(=O)(CC(F)F)Nc1cnc2cc(N3CCOCC3)nc(O[C@H]3CC[C@@H](Nc4ncccn4)CC3)c2c1]

Prepared according to general procedure N starting with 184 and 2,2-difluoroethane-1-sulfonyl chloride to afford the titled compound in 55% yield. ¹H NMR (400 MHz, DMSO) δ 10.46 (s, 1H), 8.70 (d, J=2.6 Hz, 1H), 8.34 (d, J=4.8 Hz, 2H), 8.21 (d, J=2.6 Hz, 1H), 7.46 (s, 1H), 6.63 (t, J=4.9 Hz, 1H), 6.54 (s, 1H), 6.53-6.26 (tt, J=55, 4.4 Hz, 1H), 5.36 (s, 1H), 4.03 (td, J=15.0, 4.3 Hz, 2H), 3.78-3.71 (m, 4H), 3.56-3.49 (m, 4H), 2.11 (d, J=10.8 Hz, 2H), 1.88-1.70 (m, 6H).

### 194 N-[(1s,4s)-4-({3-[(dimethylsulfamoyl)amino]-7-(morpholin-4-yl)-1,6-naphthyridin-5-yl}oxy)cyclohexyl]pyrimidin-2-amine

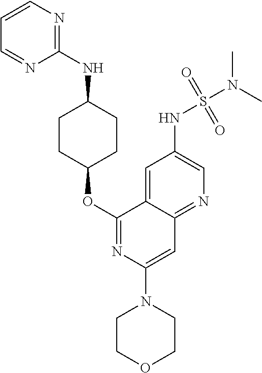 [SMILES: CN(C)S(=O)(=O)Nc1cnc2cc(N3CCOCC3)nc(O[C@H]3CC[C@@H](Nc4ncccn4)CC3)c2c1]

A mixture of 184 (1.0 eq), dimethylsulfamoyl chloride (1.1 eq), and DMAP (cat.) in pyridine (0.24M) was stirred at room temperature for 5 h. Additional portions of dimethylsulfamoyl chloride was added over 4 days until completion of the reaction. Purification via prep HPLC gave the titled compound (yield 12%). ¹H NMR (400 MHz, DMSO-d₆) δ 10.21 (s, 1H), 8.68 (d, J=2.7 Hz, 1H), 8.31 (d, J=4.8 Hz, 2H), 8.14 (d, J=2.7 Hz, 1H), 7.37 (s, 1H), 6.60 (t, J=4.8 Hz, 1H), 6.52 (s, 1H), 5.35 (s, 1H), 3.84 (s, 1H), 3.76-3.73 (m, 4H), 2.76 (s, 6H), 2.09 (d, J=9.9 Hz, 2H), 1.90-1.65 (m, 6H).

### 195 2-(7-morpholino-5-(((1s,4s)-4-(pyrimidin-2-ylamino)cyclohexyl)oxy)-1,6-naphthyridin-3-yl)isothiazolidine 1,1-dioxide

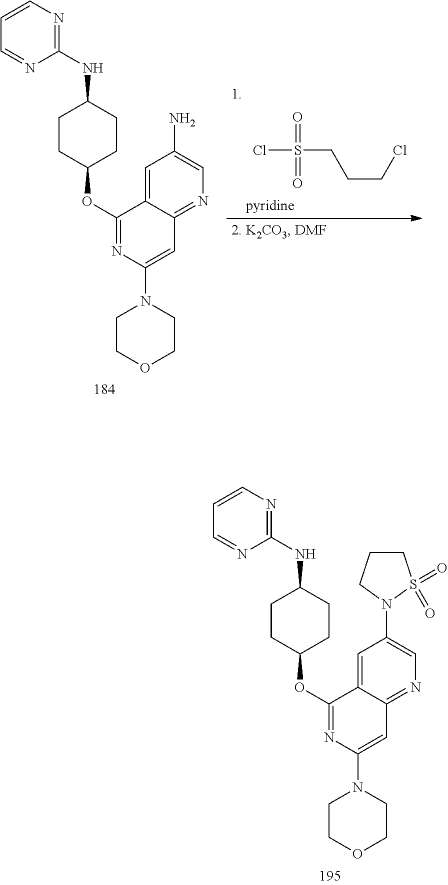 [SMILES: Nc1cnc2cc(N3CCOCC3)nc(O[C@H]3CC[C@@H](Nc4ncccn4)CC3)c2c1 ; O=S(=O)(Cl)CCCCl ; O=S1(=O)CCCN1c1cnc2cc(N3CCOCC3)nc(O[C@H]3CC[C@@H](Nc4ncccn4)CC3)c2c1]

A mixture of 184 (1.0 eq) and 3-chloropropane-1-sulfonyl chloride (1.1 eq) in pyridine (O2M) was stirred at room temperature of 2 h. Additional portions of 3-chloropropane-1-sulfonyl chloride was added over 4 h until completion of the reaction. The reaction mixture was partitioned between EtOAc and sat. NaHCO₃ aq. The organic layer was dried over Na₂SO₄ and concentrated to give 3-chloro-N-(7-morpholino-5-(((1s,4s)-4-(pyrimidin-2-ylamino)cyclohexyl)oxy)-1,6-naphthyridin-3-yl)propane-1-sulfonamide which was used without further purification.

A mixture of 3-chloro-N-(7-morpholino-5-(((1s,4s)-4-(pyrimidin-2-ylamino)cyclohexyl)oxy)-1,6-naphthyridin-3-yl)propane-1-sulfonamide (1.0 eq) and K₂CO₃ (3.0 eq) in DMF (0.08M) was stirred at room temperature for 5 h. Purification via prep HPLC gave the titled compound (yield 20%).

¹H NMR (400 MHz, DMSO) δ 8.84 (d, J=2.7 Hz, 1H), 8.34 (d, J=4.8 Hz, 2H), 8.04 (d, J=2.7 Hz, 1H), 7.50 (s, 1H), 6.63 (t, J=4.8 Hz, 1H), 6.56 (s, 1H), 5.40 (s, 1H), 3.89 (t, J=6.5 Hz, 1H), 3.75 (t, J=4.9 Hz, 4H), 3.63-3.48 (m, 8H), 2.51-2.43 (m, 2H), 2.15-2.06 (m, 2H), 1.85-1.72 (m, 6H).

### 196 1-(7-morpholino-5-(((1s,4s)-4-(pyrimidin-2-ylamino)cyclohexyl)oxy)-1,6-naphthyridin-3-yl)urea

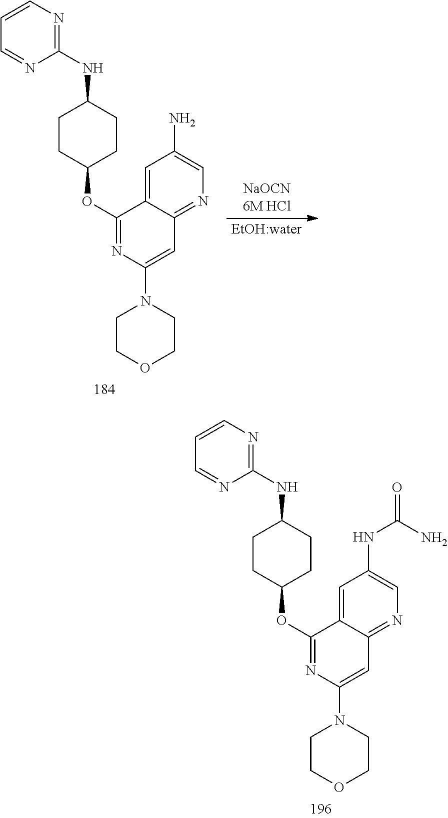 [SMILES: Nc1cnc2cc(N3CCOCC3)nc(O[C@H]3CC[C@@H](Nc4ncccn4)CC3)c2c1 ; NC(=O)Nc1cnc2cc(N3CCOCC3)nc(O[C@H]3CC[C@@H](Nc4ncccn4)CC3)c2c1]

To 184 (1.0 eq) in EtOH:water (3:1, 0.18M) was added NaOCN (1.6 eq) followed by 6M HCl (excess). The reaction mixture was stirred at room temperature for 1 h. An additional three portions of NaOCN (1.6 eq) and 6M HCl (excess) were added over 48 h until the completion of the reaction. Purification via prep HPLC gave the titled compound (yield 76%)¹H NMR (400 MHz, DMSO) δ 9.17 (s, 1H), 9.03 (s, 1H), 8.60 (s, 1H), 8.32 (d, J=4.9 Hz, 2H), 7.39 (d, J=7.1 Hz, 1H), 6.61 (t, J=4.8 Hz, 1H), 6.44 (s, 1H), 6.23 (s, 2H), 5.37 (s, 1H), 3.90-3.85 (m, 1H), 3.75 (m, J=4.9 Hz, 4H), 2.14 (m, J=11.4, 5.8 Hz, 2H), 1.91-1.66 (m, 6H). ¹³C NMR (101 MHz, DMSO) δ 160.72, 158.66, 158.28, 156.63, 156.35, 145.75, 142.59, 132.09, 125.67, 110.53, 110.23, 85.95, 72.04, 66.25, 48.69, 45.63, 28.31, 27.45.

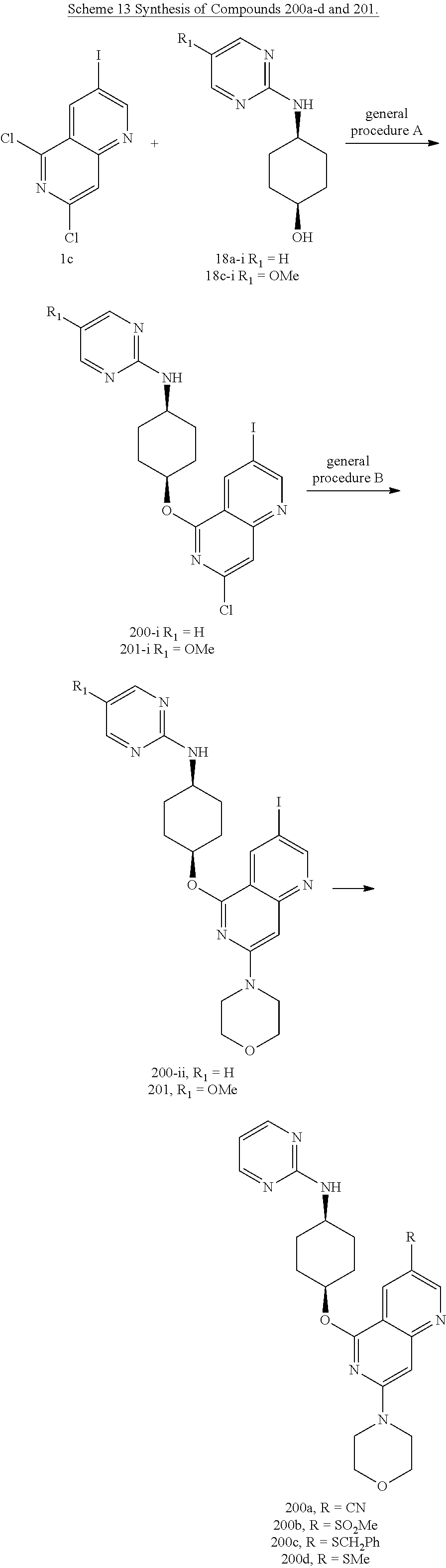

### N-((1s,4s)-4-((3-iodo-7-morpholino-1,6-naphthyridin-5-yl)oxy)cyclohexyl)pyrimidin-2-amine (200-ii)

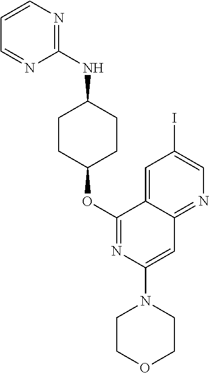 [SMILES: Ic1cnc2cc(N3CCOCC3)nc(O[C@H]3CC[C@@H](Nc4ncccn4)CC3)c2c1]

Prepared according to general procedures A and B in Scheme 13 starting with iodide 1c and alcohol 18a-i to afford the titled compound in 57% yield over 2 steps.

### 200b N-((1s,4s)-4-((3-(methylsulfonyl)-7-morpholino-1,6-naphthyridin-5-yl)oxy)cyclohexyl)pyrimidin-2-amine

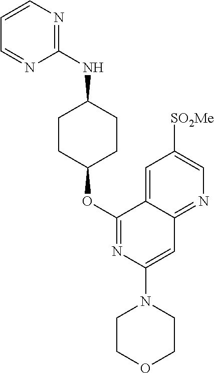 [SMILES: CS(=O)(=O)c1cnc2cc(N3CCOCC3)nc(O[C@H]3CC[C@@H](Nc4ncccn4)CC3)c2c1]

A mixture of compound 200-ii (1.0 eq), sodium methanesulfinate (2.0 eq), CuI (2.0 eq), and KOH (1.1 eq) in DMSO:water (1:0.05, 0.06M) was heated at 120° C. for 18 h. The reaction was quenched with water and the resultant precipitate was filtered. The isolated solids was washed with EtOAc:DCM and the filtrate was concentrated to give the titled compound (yield 31%)¹H NMR (400 MHz, CDCl₃) δ 9.15 (s, 1H), 8.80 (d, J=2.1 Hz, 1H), 8.32 (s, 2H), 6.58 (s, 1H), 5.43 (s, 1H), 5.25 (s, 1H), 4.03 (s, 1H), 3.91-3.84 (m, 4H), 3.72-3.65 (m, 4H), 3.19 (s, 3H), 2.25-2.18 (m, 2H), 2.05-1.99 (m, 2H), 1.99-1.75 (m, 4H). ¹³C NMR (101 MHz, DMSO) δ 162.23, 160.79, 160.08, 158.77, 158.32, 157.09, 152.38, 133.20, 129.44, 106.34, 91.80, 72.12, 66.27, 48.41, 45.39, 44.54, 28.40, 27.67.

### 200c N-((1s,4s)-4-((3-(benzylthio)-7-morpholino-1,6-naphthyridin-5-yl)oxy)cyclohexyl)pyrimidin-2-amine

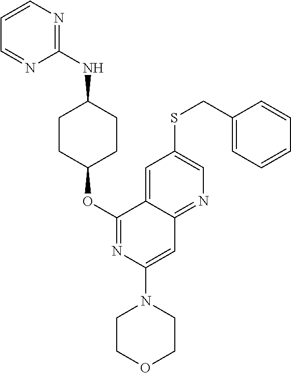 [SMILES: c1ccc(CSc2cnc3cc(N4CCOCC4)nc(O[C@H]4CC[C@@H](Nc5ncccn5)CC4)c3c2)cc1]

A mixture of compound 200-ii (1.0 eq), benzyl mercaptan (1.05 eq), Pd₂dba₃ (0.05 eq0, Xanthphos (0.1 eq), and DIEA (2.2 eq) in toluene (0.4M) was heated at 80° C. for 3 h. Purification via automated flash chromatography (20% to 80% EtOAc in hexanes) gave the titled compound (yield 90%). ¹H NMR (400 MHz, DMSO) δ 8.66 (d, J=2.4 Hz, 1H), 8.27 (d, J=4.7 Hz, 2H), 8.09 (d, J=2.5 Hz, 1H), 7.34-7.12 (m, 6H), 6.55 (t, J=4.7 Hz, 1H), 6.49 (s, 1H), 5.36 (s, 1H), 4.26 (s, 2H), 3.87 (s, 1H), 3.76-3.69 (m, 4H), 3.53-3.46 (m, 4H), 2.07 (s, 2H), 1.82-1.71 (m, 6H). ¹³C NMR (101 MHz, DMSO) δ 162.15, 158.42, 158.30, 157.35, 156.91, 154.06, 137.83, 134.20, 129.50, 128.85, 127.63, 124.51, 110.21, 108.61, 92.15, 70.74, 66.30, 48.64, 45.71, 38.83, 28.59, 27.55.

### 200d N-((1s,4s)-4-((3-(methylthio)-7-morpholino-1,6-naphthyridin-5-yl)oxy)cyclohexyl)pyrimidin-2-amine

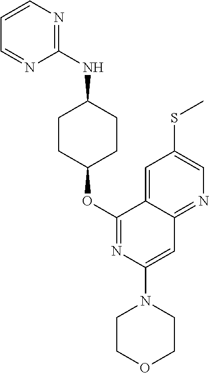 [SMILES: CSc1cnc2cc(N3CCOCC3)nc(O[C@H]3CC[C@@H](Nc4ncccn4)CC3)c2c1]

A mixture of compound 200-ii (1.0 eq), sodium methanethiolate (1.05 eq), Pd₂dba₃ (0.05 eq), Xantphos (0.1 eq), and DIEA (2.2 eq) in toluene (0.4M) was heated at 80° C. for 18 h. Additional sodium methanethiolate (4.5 eq), Pd₂dba₃ (0.05 eq), and xantphos (0.1 eq) was added and the reaction was heated at 80° C. for another 3 h. Purification via prep HPLC gave the titled compound (yield 17%). ¹H NMR (400 MHz, DMSO) δ 8.82 (d, J=2.4 Hz, 1H), 8.33 (d, J=4.9 Hz, 2H), 8.25 (d, J=2.4 Hz, 1H), 7.49 (s, 1H), 6.61 (t, J=4.8 Hz, 1H), 6.51 (s, 1H), 5.42 (s, 1H), 3.91 (s, 1H), 3.78-3.72 (m, 4H), 3.57-3.50 (m, 4H), 2.62 (s, 3H), 2.17-2.04 (m, 2H), 1.82-1.73 (m, 6H).

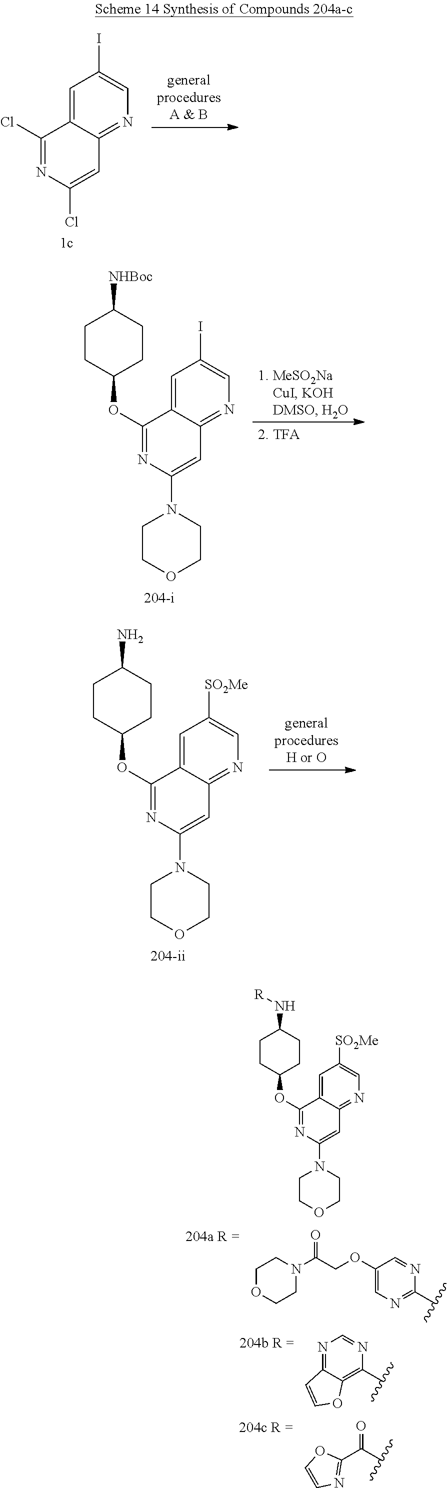

### tert-butyl ((1s,4s)-4-((3-iodo-7-morpholino-1,6-naphthyridin-5-yl)oxy)cyclohexyl)carbamate (204-i)

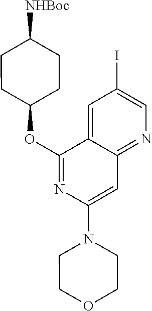 [SMILES: CC(C)(C)OC(=O)N[C@H]1CC[C@@H](Oc2nc(N3CCOCC3)cc3ncc(I)cc23)CC1]

Prepared according to general procedures A and B in Scheme 14 starting with iodide 1c and tert-butyl ((1s,4s)-4-hydroxycyclohexyl)carbamate to afford the titled compound in 82% yield over 2 steps.

### (1s,4s)-4-((3-(methylsulfonyl)-7-morpholino-1,6-naphthyridin-5-yl)oxy)cyclohexan-1-amine (204-ii)

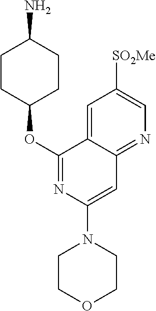 [SMILES: CS(=O)(=O)c1cnc2cc(N3CCOCC3)nc(O[C@H]3CC[C@@H](N)CC3)c2c1]

A mixture of compound 204-i (1.0 eq), sodium methanethiolate (2.0 eq), CuI (2.0 eq), and KOH (1.1 eq) in DMSO:water (1:0.05, 0.05M) was heated at 120° C. for 18 h. The reaction was partitioned between EtOAc and water. The organic layer was dried over Na₂SO₄ and concentrated to give the Boc-protected amino sulfone (yield 62%). Deprotection was accomplished using general procedure J to afford the titled compound (yield 99%) which was used in the subsequent steps without further purification.

### 204a 2-((2-(((1s,4s)-4-((3-(methylsulfonyl)-7-morpholino-1,6-naphthyridin-5-yl)oxy)cyclohexyl)amino)pyrimidin-5-yl)oxy)-1-morpholinoethan-1-one

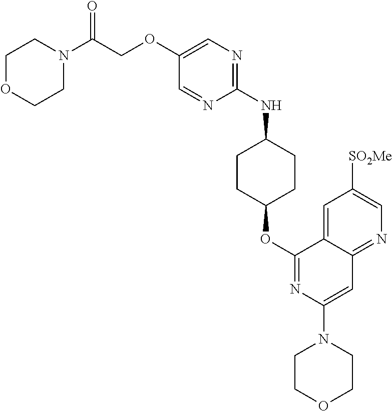 [SMILES: CS(=O)(=O)c1cnc2cc(N3CCOCC3)nc(O[C@H]3CC[C@@H](Nc4ncc(OCC(=O)N5CCOCC5)cn4)CC3)c2c1]

Prepared according to general procedure H starting with compound 204-ii and aryl chloride 61-i to afford the titled compound in 6% yield. ¹H NMR (400 MHz, DMSO) δ 9.11 (d, J=2.4 Hz, 1H), 8.63 (d, J=2.4 Hz, 1H), 8.12 (s, 2H), 6.99 (s, 1H), 6.66 (s, 1H), 5.40 (s, 1H), 4.82 (s, 2H), 3.88-3.69 (m, 5H), 3.68-3.57 (m, 4H), 3.56 (s, 8H), 3.36 (s, 3H), 2.13 (s, 2H), 1.87-1.69 (m, 6H).

### 204b N-((1s,4s)-4-((3-(methylsulfonyl)-7-morpholino-1,6-naphthyridin-5-yl)oxy)cyclohexyl)furo[3,2-d]pyrimidin-4-amine

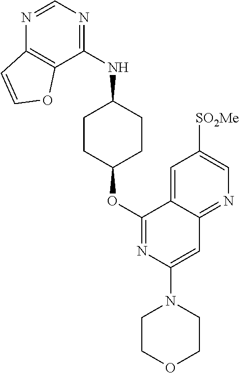 [SMILES: CS(=O)(=O)c1cnc2cc(N3CCOCC3)nc(O[C@H]3CC[C@@H](Nc4ncnc5ccoc45)CC3)c2c1]

Prepared according to general procedure H starting with compound 204-ii and 4-chlorofuro[3,2-d]pyrimidine to afford the titled compound in 6% yield. ¹H NMR (400 MHz, DMSO) δ 9.67 (s, 1H), 9.15 (d, J=2.4 Hz, 1H), 8.75 (s, 1H), 8.66 (d, J=2.4 Hz, 1H), 8.55 (d, J=2.2 Hz, 1H), 7.19 (d, J=2.2 Hz, 1H), 6.69 (s, 1H), 5.46 (s, 1H), 4.38 (s, 1H), 3.78-3.69 (m, 4H), 3.69-3.62 (m, 4H), 3.37 (s, 3H), 2.28-2.21 (m, 2H), 2.05-1.78 (m, 6H).

### 204c N-((1s,4s)-4-((3-(methylsulfonyl)-7-morpholino-1,6-naphthyridin-5-yl)oxy)cyclohexyl)oxazole-2-carboxamide

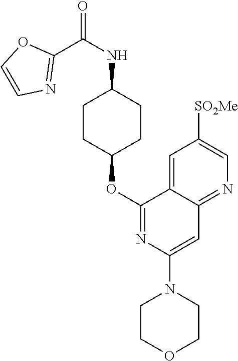 [SMILES: CS(=O)(=O)c1cnc2cc(N3CCOCC3)nc(O[C@H]3CC[C@@H](NC(=O)c4ncco4)CC3)c2c1]

Prepared according to general procedure O starting with compound 204-ii and oxazole-2-carboxylic acid to afford the titled compound in 15% yield. ¹H NMR (400 MHz, DMSO) δ 9.12 (d, J=2.4 Hz, 1H), 8.97 (d, J=8.1 Hz, 1H), 8.67 (d, J=2.4 Hz, 1H), 8.32 (s, 1H), 7.45 (s, 1H), 6.67 (s, 1H), 5.40 (s, 1H), 3.99-3.87 (m, 1H), 3.77-3.70 (m, 4H), 3.68-3.61 (m, 4H), 3.37 (s, 3H), 2.21-2.13 (m, 2H), 2.06-1.60 (m, 6H). ¹³C NMR (101 MHz, DMSO) δ 160.06, 158.76, 156.96, 155.68, 154.57, 152.36, 142.41, 133.31, 129.41, 128.52, 106.35, 91.71, 71.48, 66.28, 47.85, 45.39, 44.55, 28.49, 27.07.

### Example 4: Syntheses of Compounds

### Standard Conditions for Buchwald-Hartwig Reaction for Compounds

N-[5-(4-aminocyclohexoxy)-7-morpholino-1,6-naphthyridin-3-yl]methanesulfonamide hydrochloride (1.0 eq), the appropriate aryl chloride or bromide (1.5 eq), potassium tert-butoxide (3.0 eq), BrettPhos-Pd-G3 (0.1 eq) and BrettPhos (0.1 eq) were added to a microwave vial, which was sealed, then evacuated and back-filled with N₂ 3 times. 1,4-dioxane (2.0 mL) was added and the reaction mixture de-gassed with N₂ for 5 min. The reaction mixture was heated to 100° C. for 2 h, cooled to rt, then filtered through celite washing the pad with EtOAc (3×10 mL). The filtrate was concentrated in vacuo and the residue submitted to flash column chromatography or preparative HPLC to afford the desired product as a yellow solid.

### N-[5-[4-[[5-(4-methylpiperazin-1-yl)-2-pyridyl]amino]cyclohexoxy]-7-morpholino-1,6-naphthyridin-3-yl]methanesulfonamide (LFA2001)

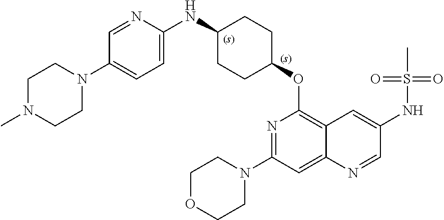 [SMILES: CN1CCN(c2ccc(N[C@H]3CC[C@@H](Oc4nc(N5CCOCC5)cc5ncc(NS(C)(=O)=O)cc45)CC3)nc2)CC1]

Prepared using a method analogous to standard conditions for the Buchwald-Hartwig reaction to afford the titled compound as a yellow solid (0.018 g, 33%).

1H NMR (500 MHz, CHLOROFORM-d), δ: 8.76 (d, J=1.72 Hz, 1H), 8.25 (d, J=1.72 Hz, 1H), 7.78 (d, J=2.86 Hz, 1H), 7.23 (dd, J=2.86, 8.59 Hz, 1H), 6.53 (s, 1H), 6.46 (d, J=8.59 Hz, 1H), 5.46 (m, 1H), 3.84-3.89 (m, 4H), 3.65-3.75 (m, 1H), 3.52-3.57 (m, 4H), 3.07 (s, 3H), 3.01-3.06 (m, 4H), 2.55-2.60 (m, 4H), 2.35 (s, 3H), 2.12-2.19 (m, 2H), 1.96 (m, 2H), 1.68-1.87 (m, 4H). LCMS: MeCN/pH1, RT=0.901 min, 80.6% (UV 254 nm), [M+H]⁺: 597.3

### Standard Conditions for Buchwald-Hartwig Reaction Followed by Deprotection

N-[5-(4-aminocyclohexoxy)-7-morpholino-1,6-naphthyridin-3-yl]methanesulfonamide; hydrochloride (LFA021) (1.0 eq), the appropriate aryl chloride or bromide (1.5 eq), potassium tert-butoxide (3.0 eq), BrettPhos-Pd-G3 (0.1 eq) and BrettPhos (0.1 eq) were added to a microwave vial, which was sealed, then evacuated and back-filled with N₂ 3 times. 1,4-dioxane (1.0 mL) was added, then the reaction mixture de-gassed with N₂ for 5 min. The reaction mixture was heated to 100° C. for 18 h, cooled to rt, then filtered through celite washing the pad with EtOAc (3×10 mL). The combined organics were concentrated in vacuo to afford a brown residue. The residue was taken up in DCM (6.0 mL) and TFA (0.5 mL) added, and the resulting solution allowed to stir at rt for 2 h. The reaction mixture was concentrated to dryness in vacuo and the residue submitted to preparative HPLC purification to afford the desired product as a yellow solid.

### N-[7-morpholino-5-[4-(5,6,7,8-tetrahydro-2,7-naphthyridin-3-ylamino)cyclohexoxy]-1,6-naphthyridin-3-yl]methanesulfonamide (LFA2002)

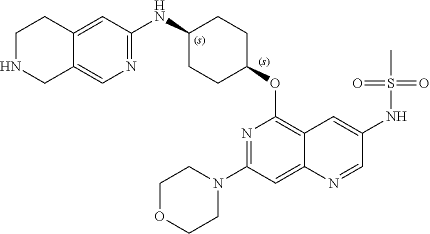 [SMILES: CS(=O)(=O)Nc1cnc2cc(N3CCOCC3)nc(O[C@H]3CC[C@@H](Nc4cc5c(cn4)CNCC5)CC3)c2c1]

Prepared using a method analogous to standard conditions for Buchwald-Hartwig reaction followed by Deprotection to afford the titled compound as a yellow solid (0.019 g, 31%).

1H NMR (500 MHz, CHLOROFORM-d), δ: 8.76 (d, J=2.29 Hz, 1H), 8.23 (d, J=2.29 Hz, 1H), 7.73 (s, 1H), 6.52 (s, 1H), 6.20 (s, 1H), 5.45 (br s, 1H), 3.89 (s, 2H), 3.82-3.86 (m, 4H), 3.61-3.71 (m, 1H), 3.50-3.56 (m, 4H), 3.10 (t, J=6.01 Hz, 2H), 3.06 (s, 3H), 2.72 (t, J=6.01 Hz, 2H), 2.11-2.18 (m, 2H), 1.91-1.97 (m, 2H), 1.69-1.86 (m, 4H). LCMS: MeCN/pH1, RT=0.865 min, 82.0% (UV 254 nm), [M+H]⁺: 554.3

### N-[7-morpholino-5-[4-(5,6,7,8-tetrahydro-1,6-naphthyridin-2-ylamino)cyclohexoxy]-1,6-naphthyridin-3-yl]methanesulfonamide (LFA2003)

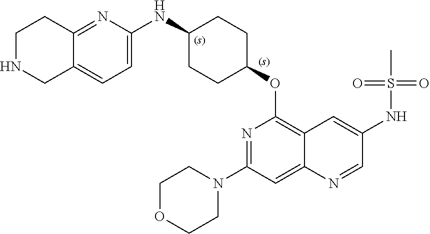 [SMILES: CS(=O)(=O)Nc1cnc2cc(N3CCOCC3)nc(O[C@H]3CC[C@@H](Nc4ccc5c(n4)CCNC5)CC3)c2c1]

Prepared using a method analogous to standard conditions for Buchwald-Hartwig reaction followed by Deprotection to afford the titled compound as a yellow solid (0.017 g, 27%).

1H NMR (500 MHz, CHLOROFORM-d), δ: 8.80 (d, J=2.29 Hz, 1H), 8.16 (d, J=2.29 Hz, 1H), 7.13 (d, J=8.02 Hz, 1H), 6.55 (s, 1H), 6.31 (d, J=8.02 Hz, 1H), 5.38-5.49 (m, 1H), 5.16 (br s, 1H), 3.89 (s, 2H), 3.83-3.88 (m, 5H), 3.66 (br s, 1H), 3.51-3.57 (m, 4H), 3.15-3.22 (m, 2H), 3.06 (s, 3H), 2.72-2.78 (m, 1H), 2.10-2.18 (m, 2H), 1.90-1.98 (m, 2H), 1.71-1.88 (m, 4H). LCMS: MeCN/pH1, RT=0.874 min, 69.6% (UV 254 nm), [M+H]⁺: 554.3

### N-[7-morpholino-5-[4-(5,6,7,8-tetrahydropyrido[3,4-d]pyrimidin-2-ylamino)cyclohexoxy]-1,6-naphthyridin-3-yl]methanesulfonamide (LFA2004)

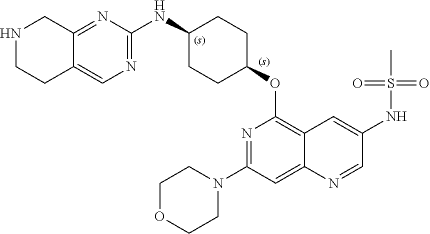 [SMILES: CS(=O)(=O)Nc1cnc2cc(N3CCOCC3)nc(O[C@H]3CC[C@@H](Nc4ncc5c(n4)CNCC5)CC3)c2c1]

Prepared using a method analogous to standard conditions for Buchwald-Hartwig reaction followed by Deprotection to afford the titled compound as a yellow solid (0.004 g, 7%).

1H NMR (500 MHz, CHLOROFORM-d), δ: 8.76 (d, J=2.86 Hz, 1H), 8.15 (d, J=2.86 Hz, 1H), 8.01 (s, 1H), 6.52 (s, 1H), 5.50 (br d, J=8.02 Hz, 1H), 5.43 (br s, 1H), 3.92-4.03 (m, 1H), 3.81-3.88 (m, 6H), 3.50-3.56 (m, 4H), 3.04-3.10 (m, 5H), 2.59 (br t, J=5.73 Hz, 2H), 2.12 (m, 2H), 1.90-1.98 (m, 2H), 1.68-1.88 (m, 4H). LCMS: MeCN/pH1, RT=0.934 min, 62.1% (UV 254 nm), [M+H]⁺: 555.3

N-[7-morpholino-5-[4-(5,6,7,8-tetrahydropyrido[4,3-d]pyrimidin-2-ylamino)cyclohexoxy]-1,6-naphthyridin-3-yl]methanesulfonamide (LFA2005)

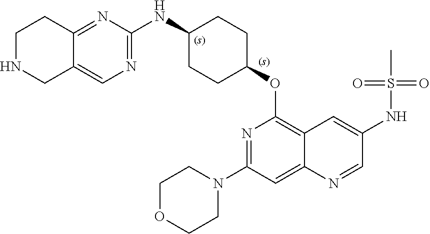 [SMILES: CS(=O)(=O)Nc1cnc2cc(N3CCOCC3)nc(O[C@H]3CC[C@@H](Nc4ncc5c(n4)CCNC5)CC3)c2c1]

Prepared using a method analogous to standard conditions for Buchwald-Hartwig reaction followed by Deprotection to afford the titled compound as a yellow solid (0.021 g, 32%).

1H NMR (500 MHz, CHLOROFORM-d), δ: 8.74 (d, J=2.29 Hz, 1H), 8.18 (d, J=2.29 Hz, 1H), 7.96 (s, 1H), 6.52 (s, 1H), 5.42 (br s, 1H), 5.26 (br d, J=8.02 Hz, 1H), 3.98 (br s, 1H), 3.80-3.92 (m, 6H), 3.49-3.57 (m, 4H), 3.16 (t, J=5.73 Hz, 2H), 3.07 (s, 3H), 2.70 (br t, J=6.01 Hz, 2H), 2.13 (br d, J=9.17 Hz, 2H), 1.95 (br d, J=8.59 Hz, 2H), 1.69-1.89 (m, 4H). LCMS: MeCN/pH1, RT=0.927 min, 70.9% (UV 254 nm), [M+H]⁺: 555.3

### N-[5-[4-[[5-(1-methylpyrrolidin-3-yl)oxy-2-pyridyl]amino]cyclohexoxy]-7-morpholino-1,6-naphthyridin-3-yl]methanesulfonamide (LFA2006)

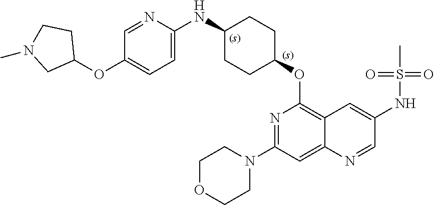 [SMILES: CN1CCC(Oc2ccc(N[C@H]3CC[C@@H](Oc4nc(N5CCOCC5)cc5ncc(NS(C)(=O)=O)cc45)CC3)nc2)C1]

Prepared using a method analogous to standard Buchwald-Hartwig reaction to afford the titled compound as a yellow solid (0.017 g, 30%).

1H NMR (500 MHz, CHLOROFORM-d), δ: 8.76 (d, J=2.86 Hz, 1H), 8.25 (d, J=2.86 Hz, 1H), 7.73 (d, J=2.86 Hz, 1H), 7.10 (dd, J=2.86, 8.88 Hz, 1H), 6.53 (s, 1H), 6.43 (d, J=8.88 Hz, 1H), 5.45 (m, 1H), 4.71 (m, 1H), 3.84-3.90 (m, 4H), 3.67-3.74 (m, 1H), 3.53-3.59 (m, 4H), 3.06 (s, 3H), 2.83-2.89 (m, 2H), 2.74 (m, 1H), 2.39-2.47 (m, 5H), 2.26 (m, 1H), 2.11-2.19 (m, 2H), 1.92-2.04 (m, 3H), 1.68-1.86 (m, 4H). LCMS: MeCN/pH1, RT=0.905 min, 75.5% (UV 254 nm), [M+H]⁺: 598.2

### 2-bromo-5-(1-methylpyrrolidin-3-yl)oxy-pyridine

To a solution of 6-bromopyridin-3-ol (0.200 g, 1.15 mmol) in DMSO (2.0 mL) was added potassium tert-butoxide (1.0 M in THF) (1.49 mL, 1.49 mmol) and the resulting solution stirred for 15 min. (1-Methylpyrrolidin-3-yl) methanesulfonate (0.309 g, 1.38 mmol) in DMSO (2.0 mL) was added and the reaction mixture stirred at 100° C. for 18 h. The reaction mixture was allowed to cool to rt, then diluted in water (20 mL). The aqueous layer was extracted with EtOAc (2×20 mL). The combined organics were dried (hydrophobic frit) then concentrated in vacuo. The residue was purified by flash column chromatography (0-80% MeCN in water pH10) to afford the titled compound as a brown oil (0.030 g, 9%).

1H NMR (500 MHz, CHLOROFORM-d), δ: 7.97 (d, J=2.86 Hz, 1H), 7.33 (d, J=8.59 Hz, 1H), 7.03 (dd, J=2.86, 8.59 Hz, 1H), 4.73-4.82 (m, 1H), 2.81-2.88 (m, 2H), 2.72 (m, 1H), 2.27-2.42 (m, 5H), 1.90-2.01 (m, 1H). LCMS: MeCN/pH1, RT=0.719 min, 88.1% (UV 254 nm), [M+H]⁺: 257.0/259.1

### (1-methylpyrrolidin-3-yl) methanesulfonate

To a solution of 1-methyl-3-pyrrolidinol (0.250 g, 2.47 mmol) in DCM (5.0 mL) was added DIPEA (0.604 mL, 3.46 mmol) and the solution cooled to 0° C. Methanesulfonyl chloride (0.230 mL, 2.97 mmol) was added dropwise and the resulting solution allowed to stir at 0° C. for 2 h. The reaction mixture was diluted with DCM (5.0 mL), and washed with sat. sodium hydrogencarbonate (10 mL). The aqueous was re-extracted with 9:1 DCM:MeOH (3×10 mL). The organics were dried (hydrophobic frit) and concentrated in vacuo to afford the titled compound as a brown oil (0.260 g, 47%).

1H NMR (500 MHz, METHANOL-d4), δ: 5.15-5.22 (m, 1H), 3.07 (s, 3H), 2.92 (m, 1H), 2.82-2.90 (m, 1H), 2.68-2.75 (m, 1H), 2.35 (s, 3H), 2.28-2.34 (m, 1H), 1.97-2.11 (m, 1H), 1.25-1.33 (m, 1H). LCMS: n/a

### Standard Conditions for Buchwald-Hartwig and SNAr Reaction

N-[5-(4-aminocyclohexoxy)-7-morpholino-1,6-naphthyridin-3-yl]methanesulfonamide; hydrochloride (1.0 eq), the appropriate aryl chloride or bromide (1.5 eq), potassium tert-butoxide (3.0 eq), BrettPhos-Pd-G3 (0.1 eq) and BrettPhos (0.1 eq) were added to a microwave vial, which was sealed, evacuated and back-filled with N₂ 3 times. 1,4-dioxane (2.0 mL) was added, then the reaction mixture de-gassed with N₂ for 5 min. The reaction mixture was heated to 100° C. for 18 h. LCMS indicated poor conversion to the desired product. The reaction mixture was allowed to cool to rt, filtered through celite washing the pad with EtOAc (3×10 mL). The filtrate was concentrated in vacuo. The residue was taken up in EtOH (1.0 mL), then triethylamine (5.0 eq) the appropriate aryl chloride or bromide (1.0 eq) were added and the reaction mixture heated to 100° C. for 4 h. The reaction mixture was concentrated in vacuo and submitted to preparative HPLC purification to afford the desired product as a yellow solid.

### N-[5-[4-[(6-methyl-7,8-dihydro-5H-pyrido[4,3-d]pyrimidin-2-yl)amino]cyclohexoxy]-7-morpholino-1,6-naphthyridin-3-yl]methanesulfonamide (LFA2007)

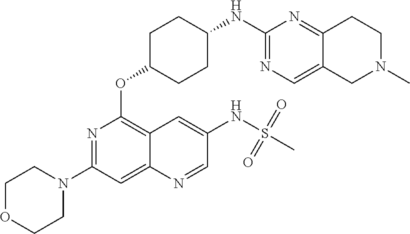

Prepared using 2-chloro-6-methyl-7,8-dihydro-5H-pyrido[4,3-d]pyrimidine and a method analogous to standard conditions for Buchwald-Hartwig and SNAr reaction to afford the titled compound as a yellow solid (0.003 g, 6%).

1H NMR (500 MHz, CHLOROFORM-d), δ: 8.78 (d, J=2.29 Hz, 1H), 8.22 (d, J=2.29 Hz, 1H), 8.02 (s, 1H), 6.53 (s, 1H), 5.89 (br s, 1H), 5.44 (br s, 1H), 3.94-4.04 (m, 1H), 3.83-3.88 (m, 4H), 3.80 (br s, 2H), 3.51-3.57 (m, 4H), 3.12 (br s, 2H), 3.06 (s, 3H), 2.98 (m, 2H), 2.72 (s, 3H), 2.12-2.19 (m, 2H), 1.90-1.97 (m, 2H), 1.71-1.85 (m, 4H). LCMS: MeCN/pH1, RT=0.942 min, 93.9% (UV 254 nm), [M+H]⁺: 569.3

2-chloro-6-methyl-7,8-dihydro-5H-pyrido[4,3-d]pyrimidine

To a solution of tert-butyl 2-chloro-7,8-dihydro-5H-pyrido[4,3-d]pyrimidine-6-carboxylate (0.250 g, 0.927 mmol) in DCM (3.0 mL) was added hydrochloric acid (3.7-4.3 M in 1,4-dioxane) (3.0 mL) and the mixture stirred at rt overnight. The mixture was concentrated to dryness in vacuo, co-evaporating with DCM (3×10 mL) to afford the desired intermediate. The residue was taken up in DCM (3.0 mL), formaldehyde (37 wt % in water) (0.345 mL, 4.63 mmol) was added dropwise and the resulting solution stirred at rt for 1 h. Sodium triacetoxyborohydride (0.982 g, 4.63 mmol) was added portionwise and the reaction mixture stirred at rt for 5 h. The reaction mixture was diluted with DCM (15 mL) and neutralised by addition of sat. NaHCO₃ (aq.) (20 mL). The layers were separated and the aqueous re-extracted with 9:1 DCM:MeOH (2×20 mL) and EtOAc (20 mL). The combined organics were dried (hydrophobic frit) and concentrated in vacuo to afford the titled compound as an off-white solid (0.140 g, 74%). 1H NMR (500 MHz, METHANOL-d4), δ: 8.40 (s, 1H), 3.61 (s, 2H), 2.94-3.00 (m, 2H), 2.79-2.86 (m, 2H), 2.48 (s, 3H). LCMS: MeCN/pH1, RT=0.325 min, 95.5% (UV 254 nm), [M+H]⁺: 184.2

### N-[5-[4-[(6-methyl-7,8-dihydro-5H-1,6-naphthyridin-2-yl)amino]cyclohexoxy]-7-morpholino-1,6-naphthyridin-3-yl]methanesulfonamide (LFA2008)

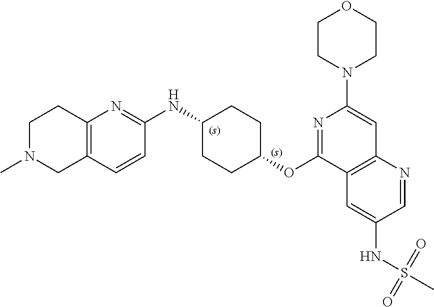

Prepared using 2-chloro-6-methyl-7,8-dihydro-5H-1,6-naphthyridine and a method analogous to standard conditions for Buchwald-Hartwig and SNAr reaction to afford the titled compound as a yellow solid (0.003 g, 60%).

1H NMR (500 MHz, CHLOROFORMV-d), δ: 8.91 (d, J=2.86 Hz, 1H), 8.22 (d, J=2.86 Hz, 1H), 7.39 (d, J=9.17 Hz, 1H), 6.55-6.60 (m, 2H), 5.50 (br s, 1H), 3.83-3.89 (m, 4H), 3.65 (br s, 2H), 3.50-3.61 (m, 5H), 3.03-3.08 (m, 5H), 2.96 (br s, 2H), 2.62 (s, 3H), 2.23 (m, 2H), 1.88-1.97 (m, 4H), 1.75 (in, 2H). LCMS: MeCN/pH1, RT=0.875 min 50.7% (UV 254 nm), [M+H]⁺: 568.2

### 2-chloro-6-methyl-7,8-dihydro-5H-1,6-naphthyridine

Prepared in an analogous method to 2-chloro-6-methyl-7,8-dihydro-5H-pyrido[4,3-d]pyrimidine, to afford the titled compound as an off-white solid (0.180 g, 850%).

1H NMR (500 MHz, METHANOL-d4), δ: 7.52 (d, J=8.02 Hz, 1H), 7.22 (d, J=8.02 Hz, 1H), 3.61 (s, 2H), 2.95-3.01 (m, 2H), 2.80-2.86 (m, 2H), 2.47 (s, 3H). LCMS: MeCN/pH1, RT=0.307 min 85.2% (UV 254 nm), [M+H]⁺: 183.1

### Standard Conditions for Attaching a Hypoxia Activated Trigger

The appropriate methyl-sulfonamide (1.0 eq), 5-(chloromethyl)-1-methyl-2-nitro-imidazole (1.5 eq), potassium carbonate (2.0 eq) and DMF or THF (1 mL) were added to a microwave vial. The mixture was stirred at rt for 18 h, then concentrated to dryness in vacuo. The residue was purified by flash column chromatography or preparative HPLC to afford the desired product as a yellow solid.

### N-[5-[4-[(3-methoxy-2-pyridyl)amino]cyclohexoxy]-7-morpholino-1,6-naphthyridin-3-yl]-N-[(3-methyl-2-nitro-imidazol-4-yl)methyl]methanesulfonamide (LFA2009)

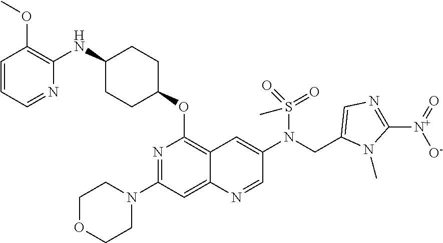 [SMILES: COc1cccnc1N[C@H]1CC[C@@H](Oc2nc(N3CCOCC3)cc3ncc(N(Cc4cnc([N+](=O)[O-])n4C)S(C)(=O)=O)cc23)CC1]

Prepared using a method analogous to standard conditions for attaching a hypoxia activated trigger to afford the titled compound as a yellow solid (0.006 g, 32%).

### N-[5-[4-[(5-fluoro-2-pyridyl)amino]cyclohexoxy]-7-morpholino-1,6-naphthyridin-3-yl]-N-[(3-methyl-2-nitro-imidazol-4-yl)methyl]methanesulfonamide (LFA2010)

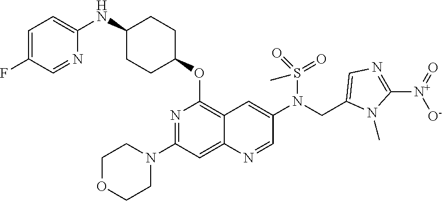 [SMILES: Cn1c(CN(c2cnc3cc(N4CCOCC4)nc(O[C@H]4CC[C@@H](Nc5ccc(F)cn5)CC4)c3c2)S(C)(=O)=O)cnc1[N+](=O)[O-]]

Prepared using a method analogous to standard conditions for attaching a hypoxia activated trigger to afford the titled compound as a yellow solid (0.002 g, 9%).

### N-[5-[4-[(5-fluoro-6-methyl-2-pyridyl)amino]cyclohexoxy]-7-morpholino-1,6-naphthyridin-3-yl]-N-[(3-methyl-2-nitro-imidazol-4-yl)methyl]methanesulfonamide (LFA2011)

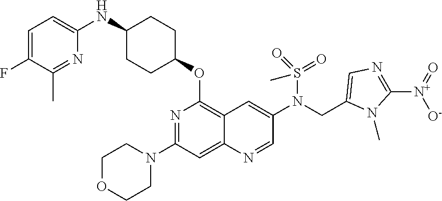 [SMILES: Cc1nc(N[C@H]2CC[C@@H](Oc3nc(N4CCOCC4)cc4ncc(N(Cc5cnc([N+](=O)[O-])n5C)S(C)(=O)=O)cc34)CC2)ccc1F]

Prepared using a method analogous to standard conditions for attaching a hypoxia activated trigger to afford the titled compound as a yellow solid (0.005 g, 53%).

### Summary of Compounds LFA2009 to LFA2011 by Headgroup

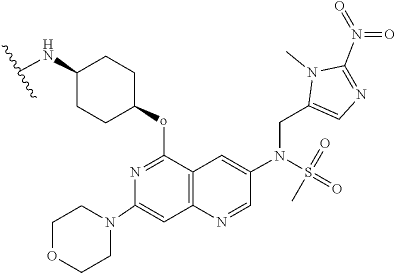

| Compound | | | LCMS RT |
|---|---|---|---|
| no. | Head Group | NMR | (MeCN/pH1) |
| LFA2009 | 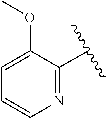 [SMILES: *c1ncccc1OC] | (CHLOROFORM-d), δ: 1.70-1.77 (m, 2H), 1.87-1.95 (m, 2H), 1.97- 2.03 (m, 2H), 2.09-2.18 (m, 2H), 3.05 (s, 3H), 3.54-3.60 (m, 4H), 3.82-3.86 (m, 7H), 4.08-4.17 (m, 4H), 5.02 (s, 3H), 5.34-5.40 (m, 1H), 6.46 (s, 1H), 6.52 (dd, J = 7.45, | 1.170 min, 96.7%, [M + H]+: 668.2 |
| | | 5.16 Hz, 1H), 6.83 (dd, J = 7.45, | |
| | | 1.15 Hz, 1H), 6.87 (s, 1H), 7.71 (dd, | |
| | | J = 5.16, 1.72 Hz, 1H), 8.08 (d, J = | |
| | | 2.29 Hz, 1H), 8.61 (d, J = 2.86 Hz, | |
| | | 1H) | |
| LFA2010 | 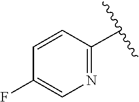 [SMILES: *c1ccc(F)cn1] | (CHLOROFORM-d), δ: 1.53-1.57 (m, 2H), 1.79-1.87 (m, 2H), 1.92- 1.99 (m, 2H), 2.09-2.17 (m, 2H), 3.06 (s, 3H), 3.55-3.60 (m, 4H), 3.81-3.87 (m, 5H), 4.11 (s, 3H), 4.82 (d, J = 8.02 Hz, 1H), 4.99 (s, 2H), 5.36 (br d, J = 2.86 Hz, 1H), 6.45- | 1.190 min, 72.5%, [M + H]+: 656.2 |
| | | 6.49 (m, 2H), 6.91 (s, 1H), 7.19 (m, | |
| | | 1H), 7.94 (d, J = 2.86 Hz, 1H), 7.96 | |
| | | (d, J = 1.72 Hz, 1H), 8.66 (d, J = | |
| | | 2.86 Hz, 1H) | |
| LFA2011 | 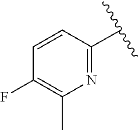 [SMILES: *c1ccc(F)c(C)n1] | (METHANOL-d4), δ: 1.67-1.75 (m, 2H), 1.82-2.02 (m, 4H), 2.12 (m, 2H), 2.36 (d, J = 2.86 Hz, 3H), 3.07 (s, 3H), 3.55-3.62 (m, 4H), 3.66-3.75 (m, 1H), 3.83-3.88 (m, 4H), 4.13 (s, 3H), 4.65 (br d, J = 7.45 Hz, 1H), 5.02 (s, 2H), 5.34-5.40 (m, 1H), 6.27 (dd, J = 8.88, 2.58 Hz, 1H), 6.48 (s, | 1.347 min, 79.0%, [M + H]+: 670.2 |
| | | 1H), 6.89 (s, 1H), 7.14 (t, J = 8.59 Hz, | |
| | | 1H), 8.02 (d, J = 2.29 Hz, 1H), 8.63 | |
| | | (d, J = 2.86 Hz, 1H) | |

For the following compounds, the following general conditions were used

### General Procedure: Buchwald-Hartwig Amination

A vial was charged with N-[5-[4-[(5-bromopyrimidin-2-yl)amino]cyclohexoxy]-7-morpholino-1,6-naphthyridin-3-yl]methanesulfonamide (1.0 eq), amine (1.5 equiv), sodium tert-butoxide (3 equiv.) tris(dibenzylideneacetone)dipalladium(0) (0.2 equiv.) and 2-(di-tert-butylphosphino)biphenyl (0.4 equiv.). The vial was capped and placed under an atmosphere of nitrogen. 1,4-dioxane (1 mL) was added and the mixture sparged with nitrogen for 10 minutes. The mixture was heated at 100 deg c for 2 hours by microwave. The mixture was cooled to room temperature then filtered through celite washing the pad with EtOAc (3×10 mL). The filtrate was concentrated in vacuo and the residue submitted to flash column chromatography or preparative HPLC to afford the desired product as a yellow solid.

### General Procedure: BOC Deprotection

BOC-protected amine (1.0 eq) was dissolved in dichloromethane (3 mL). trifluoroacetic acid (50 eq.) was added. The mixture was stirred at room temperature for 3 hours. The mixture was concentrated to dryness under vacuo and purified by either flash column chromatography or preparative HPLC to afford the desired product as a yellow solid.

### 5-(4-aminocyclohexoxy)-7-morpholino-1,6-naphthyridin-3-ol hydrochloride

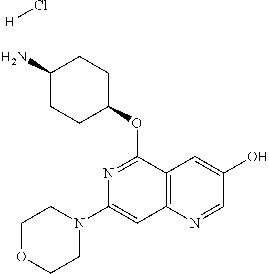

To a solution of tert-butyl n-[4-[(3-hydroxy-7-morpholino-1,6-naphthyridin-5-yl)oxy]cyclohexyl]carbamate (250 mg, 0.5624 mmol, 100 mass %) in dichloromethane (4 mL, 62.40 mmol, 100 mass %) was added hydrochloric acid (3 mol/L) in methanol (25 equiv., 14.06 mmol, 3 mol/L). The mixture was stirred at room temperature for 18 hours. the mixture was concentrated to dryness to afford 5-(4-aminocyclohexoxy)-7-morpholino-1,6-naphthyridin-3-ol hydrochloride (218 mg, 0.4579 mmol, 80 mass %, 81.41% yield).

1H NMR (500 MHz, DMSO-d6) δ 8.61-8.64 (m, 1H), 8.00-8.09 (m, 1H), 7.93-8.00 (m, 3H), 7.74-7.84 (m, 1H), 6.42-6.48 (m, 1H), 5.30-5.38 (m, 1H), 3.67-3.70 (m, 5H), 3.42-3.49 (m, 5H), 3.05-3.08 (m, 1H), 2.03-2.11 (m, 3H), 1.71-1.86 (m, 6H). LCMS pH1/MeCN RT=0.814 minutes, 41.5% (UV 254 nm), no m/z observed.

### 5-[4-[(5-bromopyrimidin-2-yl)amino]cyclohexoxy]-7-morpholino-1,6-naphthyridin-3-ol

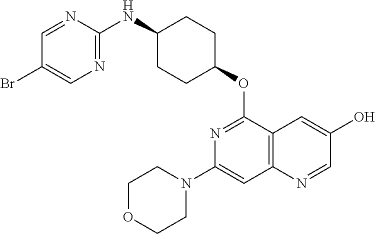 [SMILES: Oc1cnc2cc(N3CCOCC3)nc(O[C@H]3CC[C@@H](Nc4ncc(Br)cn4)CC3)c2c1]

To a solution of 5-(4-aminocyclohexoxy)-7-morpholino-1,6-naphthyridin-3-ol; hydrochloride (218 mg, 0.4579 mmol, 80 mass %) and triethylamine (3 equiv., 1.374 mmol, 100 mass %) in ethanol (5 mL, 85.9 mmol, 100 mass %) was added 5-bromo-2-chloropyrimidine (1.2 equiv., 0.5494 mmol, 98 mass %). The mixture was heated at 100° c. for 18 hours. The mixture was concentrated to dryness and purified by normal phase column chromatography (Sfar 25 g, eluting a gradient from 25% ethyl acetate 75% petroleum ether to 95% ethyl acetate 5% petroleum ether over 10CV) to afford 5-[4-[(5-bromopyrimidin-2-yl)amino]cyclohexoxy]-7-morpholino-1,6-naphthyridin-3-ol (26 mg, 0.04667 mmol, 90 mass %, 10.19% yield) as a yellow solid.

1H NMR (500 MHz, DMSO-d6) δ 9.96-10.02 (m, 1H), 8.49-8.53 (m, 1H), 8.30-8.35 (m, 2H), 7.56-7.60 (m, 1H), 7.46-7.51 (m, 1H), 6.42-6.48 (m, 1H), 5.28-5.33 (m, 1H), 3.74-3.82 (m, 1H), 3.67-3.73 (m, 4H), 3.34-3.39 (m, 4H), 2.02-2.10 (m, 2H), 1.65-1.77 (m, 6H). LCMS pH1/MeCN RT=1.509 minutes, 38.2% (UV 254 nm), [M+H]⁺: 501.1

### N-[5-[4-[(5-bromopyrimidin-2-yl)amino]cyclohexoxy]-7-morpholino-1,6-naphthyridin-3-yl]methanesulfonamide

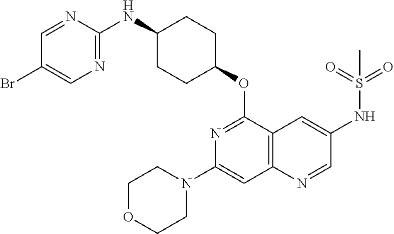 [SMILES: CS(=O)(=O)Nc1cnc2cc(N3CCOCC3)nc(O[C@H]3CC[C@@H](Nc4ncc(Br)cn4)CC3)c2c1]

To a solution of N-[5-(4-aminocyclohexoxy)-7-morpholino-1,6-naphthyridin-3-yl]methanesulfonamide; hydrochloride (250 mg, 0.5459 mmol, 100 mass %) and triethylamine (3 equiv., 1.638 mmol, 100 mass %) in ethanol (3 ml, 51.5 mmol, 100 mass %) was added 5-bromo-2-chloropyrimidine (2.5 equiv., 1.365 mmol, 98 mass %). The mixture was heated at 100° C. for 18 hours. The mixture was concentrated to dryness and redissolved in ethyl acetate (20 mL). The organic layer was washed with saturated ammonium chloride solution (2×20 mL), distilled water (20 mL) and brine (20 mL). The organic layer was dried using anhydrous sodium sulphate, filtered and concentrated to dryness to afford a brown oil. The oil was purified by normal phase column chromatography (Sfar 25 g, eluting a gradient from 0% methanol 100% dichloromethane to 10% methanol 90% dichloromethane over 10 CV) to afford N-[5-[4-[(5-bromopyrimidin-2-yl)amino]cyclohexoxy]-7-morpholino-1,6-naphthyridin-3-yl]methanesulfonamide (201 mg, 0.3127 mmol, 90 mass %, 57.29% Yield) as a yellow solid.

1H NMR (500 MHz, CHLOROFORM-d) δ 8.67-8.71 (m, 1H), 8.26-8.28 (m, 2H), 8.22-8.25 (m, 1H), 6.65-6.72 (m, 1H), 6.49-6.54 (m, 1H), 5.40-5.46 (m, 1H), 3.90-3.98 (m, 1H), 3.82-3.87 (m, 4H), 3.51-3.58 (m, 4H), 3.05-3.08 (m, 3H), 2.12-2.21 (m, 2H), 1.91-2.01 (m, 2H), 1.75-1.89 (m, 4H). LCMS: pH 1/MeCN RT=1.489 minutes, 67.8% (UV 254 nm), [M+H]+: 579.2

### 5-[4-[(5-bromopyrimidin-2-yl)amino]cyclohexoxy]-7-morpholino-1,6-naphthyridin-3-ol (LFA2012)

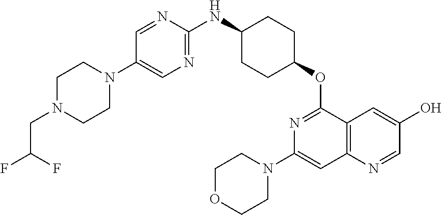 [SMILES: Oc1cnc2cc(N3CCOCC3)nc(O[C@H]3CC[C@@H](Nc4ncc(N5CCN(CC(F)F)CC5)cn4)CC3)c2c1]

Prepared using analogous method to general conditions for the Buchwald-Hartwig animation reaction using 1-(2,2-difluoroethyl)piperazine and 5-[4-[(5-bromopyrimidin-2-yl)amino]cyclohexoxy]-7-morpholino-1,6-naphthyridin-3-ol to afford title compound as a yellow solid (6.6 mg, 0.011 mmol, 95 mass %, 22% yield).

1H NMR (500 MHz, CHLOROFORM-d) δ 8.68-8.72 (m, 1H), 8.11-8.16 (m, 2H), 7.66-7.71 (m, 1H), 6.49-6.53 (m, 1H), 5.79-6.03 (m, 1H), 5.62-5.76 (m, 1H), 5.38-5.44 (m, 1H), 3.80-3.90 (m, 5H), 3.43-3.50 (m, 4H), 3.01-3.09 (m, 4H), 2.74-2.84 (m, 7H), 2.59-2.63 (m, 1H), 2.02-2.11 (m, 2H), 1.84-1.91 (m, 2H), 1.67-1.76 (m, 2H), 1.40-1.51 (m, 2H). LCMS pH1/MeCN RT=1.125 minutes, 92.2% (UV 254 nm), [M+H]+: 571.3

### N-[5-[4-[(5-bromopyrimidin-2-yl)amino]cyclohexoxy]-7-morpholino-1,6-naphthyridin-3-yl]methanesulfonamide (LFA2013)

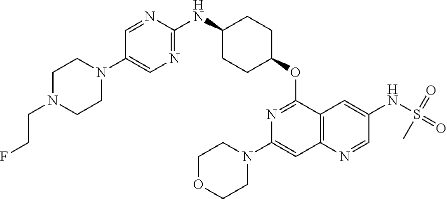 [SMILES: CS(=O)(=O)Nc1cnc2cc(N3CCOCC3)nc(O[C@H]3CC[C@@H](Nc4ncc(N5CCN(CCF)CC5)cn4)CC3)c2c1]

Prepared using analogous method to general conditions for the Buchwald-Hartwig animation reaction using 1-(2-fluoroethyl)piperazine to afford title compound as a yellow solid (16 mg, 0.02413 mmol, 95 mass %, 7.757% Yield).

1H NMR (500 MHz, CHLOROFORM-d) δ 8.69-8.75 (m, 1H), 8.21-8.26 (m, 1H), 8.04-8.11 (m, 2H), 6.48-6.54 (m, 1H), 5.46-5.54 (m, 1H), 5.41-5.46 (m, 1H), 4.48-4.72 (m, 2H), 3.87-3.98 (m, 1H), 3.81-3.88 (m, 4H), 3.49-3.56 (m, 4H), 2.72-2.86 (m, 6H), 2.72-2.86 (m, 6H), 2.12-2.18 (m, 2H), 1.93-1.99 (m, 3H), 1.82-1.88 (m, 4H). LCMS: (pH1/MeCN) RT=1.153 minutes, 85.0% (UV 254 nm), [M+H]+: 630.3

### N-[7-morpholino-5-[4-[(5-piperazin-1-ylpyrimidin-2-yl)amino]cyclohexoxy]-1,6-naphthyridin-3-yl]methanesulfonamide (LFA2014)

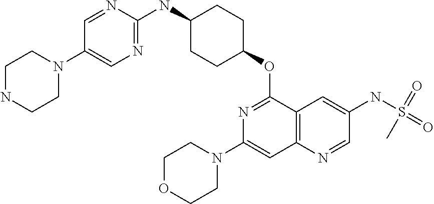 [SMILES: CS(=O)(=O)Nc1cnc2cc(N3CCOCC3)nc(O[C@H]3CC[C@@H](Nc4ncc(N5CCNCC5)cn4)CC3)c2c1]

Prepared using analogous method to general conditions for the Buchwald-Hartwig animation reaction and the Boc deprotection using 1-boc-piperazine to afford title compound as a yellow film (2.1 mg, 0.0034 mmol, 95 mass %, 4.8% Yield)

1H NMR (500 MHz, CHLOROFORM-d) δ 8.72-8.77 (m, 1H), 8.21-8.26 (m, 1H), 8.04-8.11 (m, 2H), 6.49-6.54 (m, 1H), 5.38-5.48 (m, 1H), 5.25-5.34 (m, 1H), 3.83-3.95 (m, 5H), 3.48-3.57 (m, 4H), 3.11-3.18 (m, 4H), 3.03-3.10 (m, 7H), 2.11-2.16 (m, 2H), 1.67-2.01 (m, 8H). LCMS: pH 10/MeCN RT=0.869 minutes, 80.4% (UV 254 nm), [M+H]⁺: 584.3

### N-[5-[4-[[5-(2,3,3a,4,6,6a-hexahydro-1H-pyrrolo[3,4-c]pyrrol-5-yl)pyrimidin-2-yl]amino]cyclohexoxy]-7-morpholino-1,6-naphthyridin-3-yl]methanesulfonamide (LFA2015)

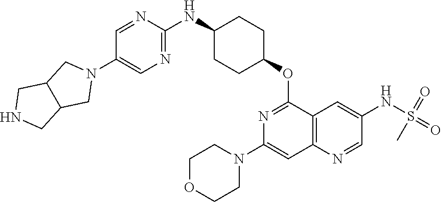 [SMILES: CS(=O)(=O)Nc1cnc2cc(N3CCOCC3)nc(O[C@H]3CC[C@@H](Nc4ncc(N5CC6CNCC6C5)cn4)CC3)c2c1]

Prepared using analogous method to general condition s for the Buchwald-Hartwig animation reaction and the Boc deprotection using tert-butyl hexahydropyrrolo[3,4-c]pyrrole-2(1 h)-carboxylate to afford title compound as a yellow solid (3.8 mg, 0.0059 mmol, 95 mass %, 8.3% yield)

1H NMR (500 MHz, CHLOROFORM-d) δ 8.71-8.76 (m, 1H), 8.18-8.23 (m, 1H), 7.86-7.90 (m, 2H), 6.49-6.53 (m, 1H), 5.38-5.46 (m, 1H), 5.00-5.09 (m, 1H), 3.84-3.93 (m, 5H), 3.50-3.55 (m, 5H), 3.10-3.23 (m, 4H), 2.99-3.08 (m, 7H), 2.85-2.90 (m, 2H), 2.11-2.18 (m, 2H), 1.94-2.00 (m, 2H), 1.72-1.87 (m, 4H). LCMS: (pH 1/MeOH) RT=1.413 minutes, 93.8% (UV 254 nm), [M+H]+: 610.3

### N-[5-[4-[[5-(3,8-diazabicyclo[3.2.1]octan-3-yl)pyrimidin-2-yl]amino]cyclohexoxy]-7-morpholino-1,6-naphthyridin-3-yl]methanesulfonamide (LFA2016)

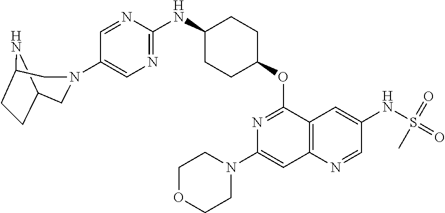 [SMILES: CS(=O)(=O)Nc1cnc2cc(N3CCOCC3)nc(O[C@H]3CC[C@@H](Nc4ncc(N5CC6CCC(C5)N6)cn4)CC3)c2c1]

Prepared using analogous method to general condition s for the Buchwald-Hartwig animation reaction and the Boc deprotection using tert-butyl 3,8-diazabicyclo[3.2.1]octane-8-carboxylate to afford title compound as a yellow solid (3.9 mg, 0.0061 mmol, 95 mass %, 8.5% yield)

1H NMR (500 MHz, CHLOROFORM-d) δ 8.62-8.76 (m, 1H), 8.11-8.22 (m, 1H), 7.90-8.00 (m, 2H), 6.43-6.54 (m, 1H), 5.37-5.47 (m, 1H), 5.08-5.36 (m, 1H), 3.79-3.93 (m, 6H), 3.44-3.63 (m, 6H), 3.07-3.20 (m, 2H), 2.99-3.07 (m, 3H), 2.74-2.87 (m, 4H), 2.06-2.16 (m, 2H), 1.76-1.96 (m, 8H). LCMS (pH1/MeCN) RT=0.956 minutes, 94.0% (UV 254 nm), [M+H]+: 611.3.

### N-[7-morpholino-5-[4-[[5-[4-(2,2,2-trifluoroethyl)piperazin-1-yl]pyrimidin-2-yl]amino]cyclohexoxy]-1,6-naphthyridin-3-yl]methanesulfonamide (LFA2017)

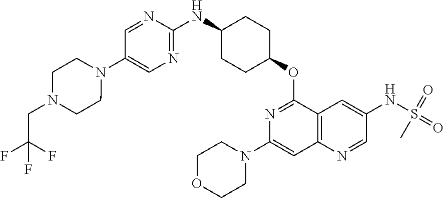 [SMILES: CS(=O)(=O)Nc1cnc2cc(N3CCOCC3)nc(O[C@H]3CC[C@@H](Nc4ncc(N5CCN(CC(F)(F)F)CC5)cn4)CC3)c2c1]

Prepared using analogous method to general condition s for the Buchwald-Hartwig animation reaction using 1-(2,2,2-trifluoroethyl)piperazine to afford title compound as an orange solid (2 mg, 0.002854 mmol, 95 mass %, 6.115% Yield).

1H NMR (500 MHz, CHLOROFORM-d) δ 8.75-8.80 (m, 1H), 8.22-8.27 (m, 1H), 8.05-8.11 (m, 2H), 6.48-6.56 (m, 1H), 5.41-5.48 (m, 1H), 3.90-4.00 (m, 1H), 3.82-3.87 (m, 4H), 3.51-3.58 (m, 4H), 3.06-3.08 (m, 3H), 2.98-3.05 (m, 6H), 2.81-2.86 (m, 4H), 2.16-2.20 (m, 2H), 1.80-1.98 (m, 6H) LCMS: pH10/MeCN RT=1.131 minutes, 93.9% (UV 254 nm), [M+H]+: 666.3

### N-[5-[4-[[5-[4-(2,2-difluoroethyl)piperazin-1-yl]pyrimidin-2-yl]amino]cyclohexoxy]-7-morpholino-1,6-naphthyridin-3-yl]methanesulfonamide (LFA2018)

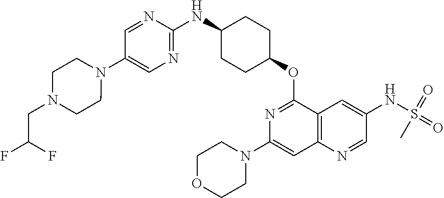 [SMILES: CS(=O)(=O)Nc1cnc2cc(N3CCOCC3)nc(O[C@H]3CC[C@@H](Nc4ncc(N5CCN(CC(F)F)CC5)cn4)CC3)c2c1]

Prepared using analogous method to general condition for the Buchwald-Hartwig animation reaction using 1-(2,2-difluoroethyl)piperazine to afford title compound as an orange solid (6.8 mg, 0.010 mmol, 95 mass %, 16% Yield).

1H NMR (500 MHz, CHLOROFORM-d) δ 8.85-8.91 (m, 1H), 8.43-8.50 (m, 1H), 7.96-8.24 (m, 2H), 6.63-6.70 (m, 1H), 5.76-6.05 (m, 1H), 5.44-5.50 (m, 1H), 3.99-4.07 (m, 2H), 3.80-3.86 (m, 4H), 3.57-3.65 (m, 4H), 3.03-3.08 (m, 8H), 2.76-2.88 (m, 6H), 2.17-2.26 (m, 2H), 1.77-2.01 (m, 6H) LCMS: (pH10/MeCN) RT=1.039 minutes, 97.9% (UV 254 nm), [M+H]+: 648.3

### N-[7-morpholino-5-[4-[[5-[4-(oxetan-3-yl)piperazin-1-yl]pyrimidin-2-yl]amino]cyclohexoxy]-1,6-naphthyridin-3-yl]methanesulfonamide (LFA2019)

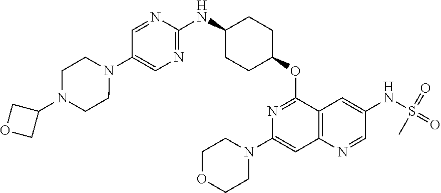 [SMILES: CS(=O)(=O)Nc1cnc2cc(N3CCOCC3)nc(O[C@H]3CC[C@@H](Nc4ncc(N5CCN(C6COC6)CC5)cn4)CC3)c2c1]

Prepared using analogous method to general condition for the Buchwald-Hartwig animation reaction using 1-(oxetan-3-yl)piperazine to afford title compound as a yellow solid (4.3 mg, 0.0064 mmol, 95 mass %, 11% Yield).

1H NMR (500 MHz, CHLOROFORM-d) δ 8.74-8.78 (m, 1H), 8.23-8.28 (m, 1H), 8.06-8.12 (m, 2H), 6.49-6.54 (m, 1H), 5.84-6.14 (m, 1H), 5.41-5.49 (m, 1H), 4.59-4.73 (m, 4H), 3.89-3.99 (m, 1H), 3.82-3.87 (m, 4H), 3.51-3.60 (m, 5H), 3.05-3.09 (m, 7H), 2.47-2.53 (m, 4H), 2.11-2.21 (m, 4H), 1.82-1.99 (m, 4H) LCMS: pH10/MeCN RT=0.904 minutes, 93.3% (UV 254 nm), [M+H]+: 640.3

### N-[7-morpholino-5-[4-[[5-(morpholinomethyl)pyrimidin-2-yl]amino]cyclohexoxy]-1,6-naphthyridin-3-yl]methanesulfonamide (LFA2020)

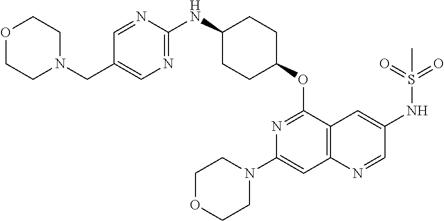 [SMILES: CS(=O)(=O)Nc1cnc2cc(N3CCOCC3)nc(O[C@H]3CC[C@@H](Nc4ncc(CN5CCOCC5)cn4)CC3)c2c1]

4-[(2-chloropyrimidin-5-yl)methyl]morpholine (38.0 mg, 0.18 mmol) and N-[5-(4-aminocyclohexoxy)-7-morpholino-1,6-naphthyridin-3-yl]methanesulfonamide LFA021 (50.0 mg, 0.12 mmol) were transferred to a 2 mL microwave vial and ethanol (0.50 mL) added followed by triethylamine (33.1 uL, 0.24 mmol). The mixture was heated in a microwave at 150° C. for 2 h and then concentrated to dryness and residue partitioned between water (20 mL) and DCM (3×10 mL). Combined extracts dried (Na₂SO₄), filtered and concentrated to dryness under reduced pressure. The residue was purified by basic prep HPLC (3×500 uL injections in DMSO). Relevant fractions were combined and concentrated to afford N-[7-morpholino-5-[4-[[5-(morpholinomethyl)pyrimidin-2-yl]amino]cyclohexoxy]-1,6-naphthyridin-3-yl]methanesulfonamide LFA2020 (11.4 mg, 16% yield) as a yellow solid.

1H NMR (400 MHz, DMSO)—9.86 (s, 1H), 8.69 (d, J=2.6 Hz, 1H), 8.17 (s, 2H), 8.11 (dd, J=2.7, 0.8 Hz, 1H), 7.08 (d, J=7.4 Hz, 1H), 6.53 (d, J=0.9 Hz, 1H), 5.37-5.31 (m, 1H), 3.91-3.80 (m, 1H), 3.77-3.70 (m, 4H), 3.58-3.51 (m, 4H), 3.51-3.44 (m, 4H), 3.26 (s, 2H), 3.02 (s, 3H), 2.35-2.29 (m, 4H), 2.15-2.06 (m, 2H), 1.84-1.68 (m, 6H). LC-MS (long, acidic method) rt 1.56 min 599.2 [M+H]+, 99.5% @254 nm

### 4-[(2-chloropyrimidin-5-yl)methyl]morpholine

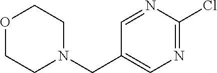 [SMILES: Clc1ncc(CN2CCOCC2)cn1]

Reference: EP3333157, 2018. 5-(bromomethyl)-2-chloro-pyrimidine (200 mg, 0.96 mmol) was dissolved in DMF (2 mL) and stirred at room temperature while potassium carbonate (266.5 mg, 1.93 mmol) was added followed by morpholine (99.8 uL, 1.16 mmol). The suspension was stirred at ambient temperature over the weekend (˜65 h). Worked up by quenching reaction mixture with water (˜20 mL), extracting with DCM (3×15 ml), washing combined extracts with brine (20 mL), drying (hydrophobic frit) and concentrating to dryness under reduced pressure. Crude 4-[(2-chloropyrimidin-5-yl)methyl]morpholine (198 mg, 96% yield) was collected as a cream coloured solid.

1H NMR (400 MHz, CDCl3)—8.59 (s, 2H), 3.74-3.67 (m, 4H), 3.49 (s, 2H), 2.49-2.42 (m, 4H).

LC-MS (long, acidic method) rt 1.04, 100% @254 nm

### N-[7-morpholino-5-[4-[[5-(1,2,3,6-tetrahydropyridin-4-yl)pyrimidin-2-yl]amino]cyclohexoxy]-1,6-naphthyridin-3-yl]methanesulfonamide

### (LFA2021)

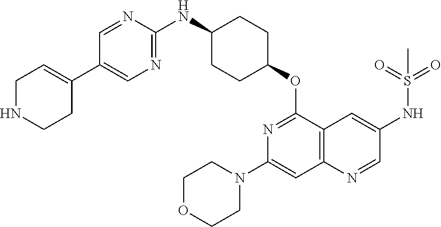 [SMILES: CS(=O)(=O)Nc1cnc2cc(N3CCOCC3)nc(O[C@H]3CC[C@@H](Nc4ncc(C5=CCNCC5)cn4)CC3)c2c1]

Trifluoroacetic acid (0.03 mL, 0.42 mmol) was added to a stirred solution of tert-butyl-4-[2-[[4-[[3-(methanesulfonamido)-7-morpholino-1,6-naphthyridin-5-yl]oxy]cyclohexyl]amino]pyrimidin-5-yl]-3,6-dihydro-2H-pyridine-1-carboxylate (30 mg, 0.04 mmol) in DCM (2 mL). The mixture was stirred at RT for 4 h. A further portion of Trifluoroacetic acid (0.1 mL, 1.31 mmol) was added and the mixture was stirred at RT for 18 h. The mixture was concentrated in vacuo and the residue was re-dissolved in MeOH (1 mL) and loaded directly onto an SCX column (1 g). The column was flushed with MeOH (100 mL) followed by 2 M NH/MeOH (503 mL). The NH₃/MeOH fraction was concentrated in vacuo to give the crude product (30 mg) as a yellow/orange gum. The yellow/orange gum was purified by basic prep. HPLC (method: basic prep.; 5-95% 0.005 M NH₄OH/MeCN in 0.005 M NH₄OH/H₂O) to give the product, N-[7-morpholino-5-[4-[[5-(1,2,3,6-tetrahydropyridin-4-yl)pyrimidin-2-yl]amino]cyclohexoxy]-1,6-naphthyridin-3-yl]methanesulfonamide LFA2021 (15 mg, 62% yield), as a yellow solid.

1H NMR (400 MHz, DMSO-d6)—8.65 (d, J=2.7 Hz, 1H), 8.36 (s, 2H), 8.05 (d, J=2.6 Hz, 1H), 7.17 (d, J=7.5 Hz, 1H), 6.51 (s, 1H), 6.07 (td, J=3.5, 1.8 Hz, 1H), 5.39-5.26 (m, 1H), 3.93-3.82 (m, 1H), 3.74 (dd, J=6.0, 3.9 Hz, 4H), 3.50-3.42 (m, 4H), 3.39-3.36 (m, 2H), 2.96 (s, 3H), 2.94 (t, J=5.7 Hz, 2H), 2.39-2.25 (m, 2H), 2.10 (s, 2H), 1.91-1.64 (m, 6H). LCMS (long, acidic): 1.65 min, 581.2 [M+H]; 99% purity @254 nm.

### tert-butyl 4-[2-[[4-[[3-(methanesulfonamido)-7-morpholino-1,6-naphthyridin-5-yl]oxy]cyclohexyl]amino]pyrimidin-5-yl]-3,6-dihydro-2H-pyridine-1-carboxylate

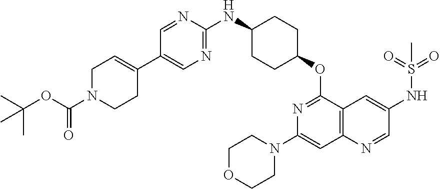 [SMILES: CC(C)(C)OC(=O)N1CC=C(c2cnc(N[C@H]3CC[C@@H](Oc4nc(N5CCOCC5)cc5ncc(NS(C)(=O)=O)cc45)CC3)nc2)CC1]

N-[5-[4-[(5-bromopyrimidin-2-yl)amino]cyclohexoxy]-7-morpholino-1,6-naphthyridin-3-yl]methanesulfonamide (100 mg, 0.17 mmol), tert-butyl4-(4,4,5,5-tetramethyl-1,3,2-dioxaborolan-2-yl)-3,6-dihydro-2H-pyridine-1-carboxylate (64.1 mg, 0.21 mmol), 1,1′-Bis(diphenylphosphino)ferrocenepalladium(II) dichloride (6.3 mg, 0.01 mmol) and potassium carbonate (71.7 mg, 0.52 mmol) were charged to a 2-5 mL microwave vial. The vial was sealed and the mixture was purged with a stream of argon for 5 min. 1,4-Dioxane (1.5 mL) and water (0.3 mL) were added and the mixture was sparged with argon for 5 min. The mixture was heated to 90° C. and stirred for 16 h. The mixture was diluted with EtOAc (2 mL) and filtered through a pad of Celite, eluting with EtOAc (50 mL). The filtrate was concentrated in vacuo to give the crude product as a brown/black gum (172 mg). The crude product was purified by flash column chromatography (10 g silica column, eluting with 0-5% MeOH in DCM) to give the product, tert-butyl4-[2-[[4-[[3-(methanesulfonamido)-7-morpholino-1,6-naphthyridin-5-yl]oxy]cyclohexyl]amino]pyrimidin-5-yl]-3,6-dihydro-2H-pyridine-1-carboxylate (110 mg, 93% yield), as a yellow/orange solid.

1H NMR (400 MHz, Methanol-d4)—8.64 (d, J=2.6 Hz, 1H), 8.33 (s, 2H), 8.32 (dd, J=2.7, 0.8 Hz, 1H), 6.46 (d, J=0.9 Hz, 1H), 5.98 (br. s, 1H), 5.47-5.39 (m, 1H), 4.02 (br. s, 2H), 3.92 (s, 1H), 3.81 (dd, J=5.8, 4.0 Hz, 4H), 3.64-3.57 (m, 2H), 3.56-3.49 (m, 4H), 3.03 (s, 3H), 2.53-2.36 (m, 2H), 2.34-2.07 (m, 2H), 2.00-1.72 (m, 6H), 1.48 (s, 9H). LCMS ( ) (short, acidic): 1.84 min, 681.3 [M+H]; 98% purity @254 nm

### N-[7-morpholino-5-[4-[[5-(2-piperazin-1-ylethoxy)pyrimidin-2-yl]amino]cyclohexoxy]-1,6-naphthyridin-3-yl]methanesulfonamide (LFA2022)

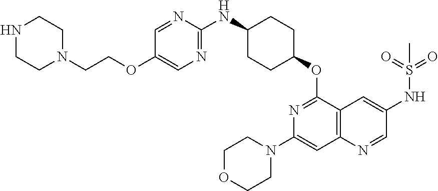 [SMILES: CS(=O)(=O)Nc1cnc2cc(N3CCOCC3)nc(O[C@H]3CC[C@@H](Nc4ncc(OCCN5CCNCC5)cn4)CC3)c2c1]

Hydrochloric acid (3.7-4.3 M in 1,4-dioxane) (0.3 mL, 1.2 mmol) was added to a stirred solution of tert-butyl4-[2-[2-[[4-[[3-(methanesulfonamido)-7-morpholino-1,6-naphthyridin-5-yl]oxy]cyclohexyl]amino]pyrimidin-5-yl]oxyethyl]piperazine-1-carboxylate (87 mg, 0.12 mmol) in MeOH (2 mL). The mixture was stirred at RT for 18 h. The solvent was removed in vacuo and the residue was re-dissolved in MeOH (1.5 mL) and was loaded directly onto an SCX column (1 g). The column was flushed with MeOH (100 mL) followed by 2 M NH/MeOH (503 mL). The NH/MeOH fraction was concentrated in vacuo to give the crude product (61 mg) as a yellow/orange gum/glassy solid. The crude product was purified by basic prep. HPLC (method: basic prep.; 5-95% 0.005 M NHOH/MeCN in 0.005 M NHOH/HO) to give the product, N-[7-morpholino-5-[4-[[5-(2-piperazin-1-ylethoxy)pyrimidin-2-yl]amino]cyclohexoxy]-1,6-naphthyridin-3-yl]methanesulfonamide LFA2022 (41 mg, 55% yield), as a yellow solid.

1H NMR (400 MHz, DMSO-d6)—8.65 (d, J=2.6 Hz, 1H), 8.11 (s, 2H), 8.06 (dd, J=2.6, 0.7 Hz, 1H), 6.70 (d, J=7.4 Hz, 1H), 6.51 (d, J=0.9 Hz, 1H), 5.38-5.28 (m, 1H), 4.03 (t, J=5.8 Hz, 2H), 3.83-3.67 (m, 5H), 3.49-3.40 (m, 4H), 2.97 (s, 3H), 2.76-2.69 (m, 4H), 2.61 (t, J=5.8 Hz, 2H), 2.45-2.36 (m, 4H), 2.16-2.01 (m, 2H), 1.87-1.61 (m, 6H).

LCMS (long, acidic): 1.55 min, 628.3 [M+H]; 97% purity @254 nm

### tert-butyl 4-[2-[2-[[4-[[3-(methanesulfonamido)-7-morpholino-1,6-naphthyridin-5-yl]oxy]cyclohexyl]amino]pyrimidin-5-yl]oxyethyl]piperazine-1-carboxylate

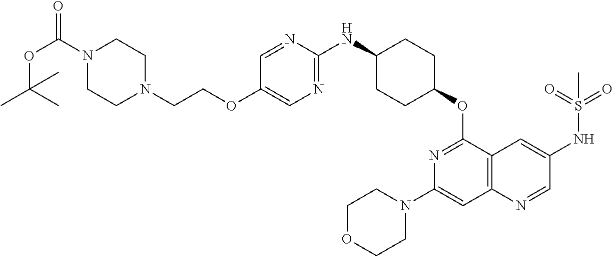 [SMILES: CC(C)(C)OC(=O)N1CCN(CCOc2cnc(N[C@H]3CC[C@@H](Oc4nc(N5CCOCC5)cc5ncc(NS(C)(=O)=O)cc45)CC3)nc2)CC1]

N-[5-(4-aminocyclohexoxy)-7-morpholino-1,6-naphthyridin-3-yl]methanesulfonamide LFA021 (150 mg, 0.36 mmol), tert-butyl 4-[2-(2-chloropyrimidin-5-yl)oxyethyl]piperazine-1-carboxylate (305 mg, 0.89 mmol), potassium tert-butoxide (119.8 mg, 1.07 mmol) and BrettPhos Pd G3 (64.5 mg, 0.07 mmol) were charged to a 20 mL microwave vial. The vial was sealed and purged with a stream of argon for 5 min. 1,4-dioxane (3.6 mL) was added and the mixture was sparged with argon for 5 min. The mixture was heated to 100° C. and stirred for 16 h. After cooling to RT, further portions of tert-butyl 4-[2-(2-chloropyrimidin-5-yl)oxyethyl]piperazine-1-carboxylate (58 mg, 0.17 mmol), potassium tert-butoxide (100 mg, 0.89 mmol) and BrettPhos Pd G3 (64.5 mg, 0.07 mmol) were added. The vial was re-sealed and was sparged with argon for 5 min. The mixture was heated to 100° C. and stirred for 6 h, then at RT over the weekend. The reaction mixture was diluted with EtOAc (2 mL) and filtered through a short pad of Celite, eluting with EtOAc (50 mL). The filtrate was concentrated in vacuo to give a green/black gum (600 mg). The green/black gum was dissolved in MeOH was loaded directly onto an SCX column (2 g). The column was flushed with MeOH (150 mL) followed by 2 M NH₃/MeOH (60 mL). The NH₃/MeOH fraction was concentrated in vacuo to give an orange gum (110 mg). Purified by flash column chromatography (10 g silica column, eluting with 0-8% EtOAc in MeOH) to give the product, tert-butyl 4-[2-[2-[[4-[[3-(methanesulfonamido)-7-morpholino-1,6-naphthyridin-5-yl]oxy]cyclohexyl]amino]pyrimidin-5-yl]oxyethyl]piperazine-1-carboxylate xxxx (87 mg, 34%) yield), as an orange gum/glass. 1H NMR (400 MHz, Methanol-d4)—8.65 (d, J=2.7 Hz, 1H), 8.33 (dd, J=2.7, 0.8 Hz, 1H), 8.10 (s, 2H), 6.48 (d, J=0.9 Hz, 1H), 5.48-5.42 (m, 1H), 4.12 (t, J=5.5 Hz, 2H), 3.89-3.79 (m, 5H), 3.58-3.52 (m, 4H), 3.50-3.39 (m, 8H), 3.03 (s, 3H), 2.79 (t, J=5.7 Hz, 2H), 2.25-2.12 (m, 2H), 1.97-1.74 (m, 6H), 1.45 (s, 9H)

LCMS (short, acidic): 1.39 min, 728.3 [M+H]; 94% purity @254 nm

### tert-butyl4-[2-(2-chloropyrimidin-5-yl)oxyethyl]piperazine-1-carboxylate

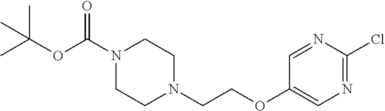 [SMILES: CC(C)(C)OC(=O)N1CCN(CCOc2cnc(Cl)nc2)CC1]

2-chloropyrimidin-5-ol (O2 g, 1.53 mmol) and Triphenylphosphine (0.8 g, 3.06 mmol) were added to a stirred solution of tert-butyl 4-(2-hydroxyethyl)piperazine-1-carboxylate (0.53 g, 2.3 mmol) in THF (8 mL). The mixture was ice-cooled before Di-tert-butylazodicarboxylate (0.53 g, 2.3 mmol) was added in a single portion. The mixture was allowed to warm to RT and stirred for 64 h. The mixture was concentrated in vacuo and the residue was purified by flash column chromatography (25 g silica column, eluting with 50-100% EtOAc in n-heptane) to give an off-white solid (504 mg). 1H NMR was consistent with the product structure at 89% purity (11 wt % Ph₃PO).

The solid was re-dissolved in MeOH (5 mL) and was loaded directly onto an SCX column (5 g). The column was flushed with MeOH (220 mL) followed by 2 M NH₃/MeOH (160 mL). The NH₃/MeOH fraction was concentrated in vacuo to give the product, tert-butyl 4-[2-(2-chloropyrimidin-5-yl)oxyethyl]piperazine-1-carboxylate (0.434 g, 83% yield), as a white solid.

1H NMR (400 MHz, DMSO-d6)—8.55 (s, 2H), 4.26 (t, J=5.6 Hz, 2H), 3.36-3.24 (m, 4H), 2.73 (t, J=5.6 Hz, 2H), 2.46-2.37 (m, 4H), 1.39 (s, 9H).

LCMS (short, acidic): 1.22 min, 287.2/289.2 [M−Bu+H]; 97% purity @254 nm.

### N-[7-morpholino-5-[4-[[5-(2-morpholinoethoxy)pyrimidin-2-yl]amino]cyclohexoxy]-1,6-naphthyridin-3-yl]methanesulfonamide (LFA2023)

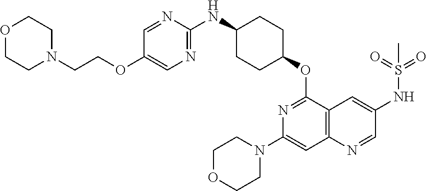 [SMILES: CS(=O)(=O)Nc1cnc2cc(N3CCOCC3)nc(O[C@H]3CC[C@@H](Nc4ncc(OCCN5CCOCC5)cn4)CC3)c2c1]

A 5 mL microwave vial was charged with N-[5-(4-aminocyclohexoxy)-7-morpholino-1,6-naphthyridin-3-yl]methanesulfonamide LFA021 (100.0 mg, 0.24 mmol), 4-[2-(2-chloropyrimidin-5-yl)oxyethyl]morpholine (86.7 mg, 0.36 mmol), potassium tert-butoxide (79.9 mg, 0.71 mmol) and BrettPhos Pd G3 (21.5 mg, 0.02 mmol). The vial was sealed and the mixture was purged with a stream of argon for 5 min. 1,4-dioxane (2.4 mL) was added and the mixture was degassed by sparging with argon for 5 min. The mixtures were heated to 90° C. and stirred for 18 h. After cooling to RT, further portions of 4-[2-(2-chloropyrimidin-5-yl)oxyethyl]morpholine (57.8 mg, 0.24 mmol), potassium tert-butoxide (53.2 mg, 0.47 mmol) and BrettPhos Pd G3 (10.8 mg, 0.01 mmol) were added and the mixture was degassed by sparging with argon for 5 min. The mixture was re-heated to 90° C. and stirred for 18 h. The reaction mixture was diluted with EtOAc (2 mL) and filtered through a short pad of Celite, eluting with EtOAc (50 mL). The filtrate was concentrated in vacuo to give an orange/brown gum (294 mg). The orange/brown gum was re-dissolved in MeOH (2 mL) and a few drops of AcOH were added. The mixture was loaded directly onto an SCX column (2 g). The column was flushed with MeOH (120 mL) followed by 2M NH/MeOH (60 mL). The NH/MeOH fraction was concentrated in vacuo to give the crude product as an orange/brown gum (125 mg). The crude product was purified by flash column chromatography (20 g silica column, eluting with 0-10% 2 M NH/MeOH in DCM) to give impure product as a yellow gum/glassy solid (29 mg). The impure product was further purified by basic prep. HPLC (method: basic prep.; 5-95% 0.005 M NHOH/MeCN in 0.005 M NH₄OH/HO) to give the product, 2N-[7-morpholino-5-[4-[[5-(2-morpholinoethoxy)pyrimidin-2-yl]amino]cyclohexoxy]-1,6-naphthyridin-3-yl]methanesulfonamide LFA2023, 20 mg, 13% yield), as a yellow solid.

1H NMR (400 MHz, DMSO—) was consistent with the product structure at >95% purity.d61H NMR (400 MHz, DMSO-d6)—9.88 (br. s, 1H), 8.70 (d, J=2.7 Hz, 1H), 8.13 (dd, J=2.8, 0.7 Hz, 1H), 8.12 (s, 2H), 6.71 (d, J=7.4 Hz, 1H), 6.53 (d, J=0.9 Hz, 1H), 5.37-5.28 (m, 1H), 4.05 (t, J=5.7 Hz, 2H), 3.85-3.68 (m, 5H), 3.61-3.52 (m, 4H), 3.52-3.43 (m, 4H), 3.03 (s, 3H), 2.63 (t, J=5.7 Hz, 2H), 2.44 (dd, J=5.7, 3.7 Hz, 4H), 2.17-2.03 (m, 2H), 1.89-1.62 (m, 6H).

LCMS (long, acidic): 1.59 min, 629.2 [M+H]; 99% purity @254 nm.

### 4-[2-(2-chloropyrimidin-5-yl)oxyethyl]morpholine

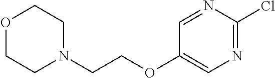 [SMILES: Clc1ncc(OCCN2CCOCC2)cn1]

To an ice-cooled stirred solution of 2-chloropyrimidin-5-ol (0.500 g, 3.83 mmol, 1.00 eq), 2-morpholinoethanol (0.700 mL, 5.74 mmol, 1.50 eq) and triphenylphosphine (2.00 g, 7.66 mmol, 2.00 eq) in tetrahydrofuran (19 mL) was added in one portion di-tert-butylazodicarboxylate (1.32 g, 5.75 mmol, 1.50 eq) and the reaction mixture allowed to stir at room temperature for 16 h. The volatiles were removed in vacuo and the crude reaction mixture was purified by flash column chromatography, eluting with 50-100% ethyl acetate/n-heptane, giving a colourless oil. The product was obtained through trituration from ethyl acetate/n-heptane as a white crystalline solid (901 mg, 97%).

1H NMR (400 MHz, DMSO-d)—8.55 (s, 2H), 4.26 (t, J=5.6 Hz, 2H), 3.59-3.51 (m, 4H), 2.70 (t, J=5.6 Hz, 2H), 2.48-2.42 (m, 4H)

### N-[5-[4-[[5-[2-(dimethylamino)ethoxy]pyrimidin-2-yl]amino]cyclohexoxy]-7-morpholino-1,6-naphthyridin-3-yl]methanesulfonamide (LFA2024)

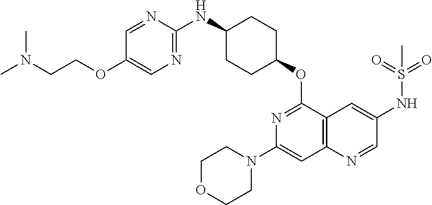 [SMILES: CN(C)CCOc1cnc(N[C@H]2CC[C@@H](Oc3nc(N4CCOCC4)cc4ncc(NS(C)(=O)=O)cc34)CC2)nc1]

A 5 mL microwave vial was charged with N-[5-(4-aminocyclohexoxy)-7-morpholino-1,6-naphthyridin-3-yl]methanesulfonamide LFA021 (75.0 mg, 0.18 mmol), potassium tert-butoxide (59.9 mg, 0.53 mmol) and BrettPhos (9.6 mg, 0.02 mmol) BrettPhos Pd G3 (8.1 mg, 0.01 mmol). The vial was sealed and the mixture was purged with a stream of argon for 5 min. 1,4-dioxane (2.4 mL) was added and the mixture was degassed by sparging with argon for 5 min. The mixture was heated to 90° C. and stirred for 24 h. Further 2-(2-chloropyrimidin-5-yl)oxy-N,N-dimethyl-ethanamine (53.8 mg, 0.27 mmol) and BrettPhos (4.8 mg, 0.01 mmol) were added, the reaction purged with argon for 5 minutes and heated for 4 hours. The reaction mixture was diluted with EtOAc (2 mL) and filtered through a short pad of Celite, eluting with EtOAc and methanol. The filtrate was concentrated in vacuo. The resulting residue was purified by acidic prep HPLC. The fractions were loaded onto a 1 g SCX. The column was washed with methanol and eluted with 2M ammonia in methanol. The ammonia fraction was concentrated at reduced pressure and purified by basic prep HPLC. The relevant fractions were combined and the solvent removed at reduced pressure to afford LFA2024 16.2 mg, 0.028 mmol, 16% yield

1H NMR (400 MHz, DMSO)—9.86 (s, 1H), 8.70 (d, J=2.7 Hz, 1H), 8.13 (dd, J=2.6, 0.8 Hz, 1H), 8.11 (s, 2H), 6.71 (d, J=7.4 Hz, 1H), 6.55-6.52 (m, 1H), 5.34 (s, 1H), 4.02 (t, J=5.7 Hz, 2H), 3.80-3.68 (m, 5H), 3.51-3.44 (m, 4H), 3.03 (s, 3H), 2.60 (t, J=5.6 Hz, 2H), 2.22 (s, 6H), 2.13-2.05 (m, 2H), 1.84-1.68 (m, 6H). LC-MS (acidic long method) RT=1.547 min, m/z 587.23 (M+H)

### 2-(2-chloropyrimidin-5-yl)oxy-N,N-dimethyl-ethanamine

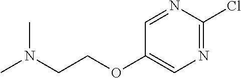

2-chloropyrimidin-5-ol (0.97 g, 7.43 mmol), 2-chloro-N,N-dimethyl-ethanamine; hydrochloride (3.21 g, 22.29 mmol), caesium carbonate (7.26 g, 22.29 mmol) and potassium iodide (1.23 g, 7.43 mmol) was stirred in DMF (15 mL) and MeCN (15 mL) under an argon atmosphere. The reaction was heated to 90° C. for 5 hours and then cooled to room temperature, diluted with DCM (50 mL), washed with water (100 mL) and the aqueous layer extracted with further DCM (2×50 mL). The combined organics was dried (hydrophobic frit) and concentrated at reduced pressure. The resulting residue containing DMF was loaded onto a 10 g SCX, the column washed with methanol and eluted with 2Mammonia in methanol. The ammonia fraction was concentrated at reduced pressure to afford 2-(2-chloropyrimidin-5-yl)oxy-N,N-dimethyl-ethanamine (884 mg, 59% yield) as an orange/brown solid.

1H NMR (400 MHz, CDCl3) δ: 8.32 (s, 2H), 4.14 (t, J=5.5 Hz, 2H), 2.75 (t, J=5.5 Hz, 2H), 2.34 (s, 6H).

### N-[5-[4-[[5-(1-methylpyrrolidin-3-yl)oxypyrimidin-2-yl]amino]cyclohexoxy]-7-morpholino-1,6-naphthyridin-3-yl]methanesulfonamide (LFA2025)

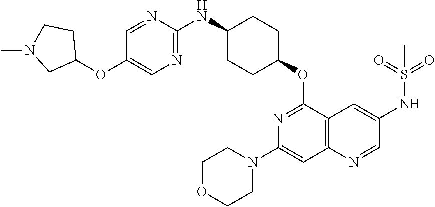 [SMILES: CN1CCC(Oc2cnc(N[C@H]3CC[C@@H](Oc4nc(N5CCOCC5)cc5ncc(NS(C)(=O)=O)cc45)CC3)nc2)C1]

1H NMR (400 MHz, DMSO)—9.86 (s, 1H), 8.70 (d, J=2.6 Hz, 1H), 8.13 (d, J=2.6 Hz, 1H), 8.05 (s, 2H), 6.71 (d, J=7.3 Hz, 1H), 6.53 (s, 1H), 5.38-5.30 (m, 1H), 4.80-4.71 (m, 1H), 3.83-3.68 (m, 5H), 3.51-3.44 (m, 4H), 3.03 (s, 3H), 2.75-2.58 (m, 3H), 2.39-2.31 (m, 1H), 2.25 (s, 3H), 2.24-2.14 (m, 1H), 2.13-2.05 (m, 2H), 1.86-1.65 (m, 7H). LC-MS (long, acidic method) rt 1.62 min, 599.2 [M+H]+, 98.3% purity @ 254 nm N-[5-[4-[[5-(1-methylpyrrolidin-3-yl)oxypyrimidin-2-yl]amino]cyclohexoxy]-7-morpholino-1,6-naphthyridin-3-yl]methanesulfonamide (31.9 mg, 0.0533 mmol, 100% yield)

A 2-5 mL microwave vial was charged with N-[5-(4-aminocyclohexoxy)-7-morpholino-1,6-naphthyridin-3-yl]methanesulfonamide (350 mg, 0.83 mmol), 2-chloro-5-(1-methylpyrrolidin-3-yl)oxy-pyrimidine (266.1 mg, 1.25 mmol), potassium tert-butoxide (279.5 mg, 2.49 mmol) and tBuBrettPhos Pd G3 (71.0 mg, 0.08 mmol). The vial was sealed and the mixture was purged with a stream of argon for 5 min. 1,4-Dioxane (16 mL) was added and the mixture was degassed by sparging with argon for 5 min. The mixture was heated to 100° C. with stirring for 5 h. After cooling to RT the reaction mixture was filtered through Dicalite, eluting with DCM and MeOH. The filtrate was concentrated in vacuo. The resulting residue was purified by flash chromatography (40 g SiO) eluting with a gradient 0-10% of 10% 2M ammonia in methanol in DCM. The appropriate fractions were combined and the solvent removed at reduced pressure. 1H NMR Contains minor impurities but is consistent with the desired product. The appropriate fractions were loaded onto a 2 g SCX. The column was washed with methanol and eluted with 2M ammonia in methanol. The ammonia fraction was concentrated at reduced pressure to afford LFA2025.

1H NMR (400 MHz, DMSO)—9.86 (s, 1H), 8.70 (d, J=2.7 Hz, 1H), 8.13 (d, J=2.7 Hz, 1H), 8.05 (s, 2H), 6.74-6.67 (m, 1H), 6.53 (s, 1H), 5.34 (s, 1H), 4.80-4.71 (m, 1H), 3.81-3.70 (m, 5H), 3.51-3.44 (m, 4H), 3.03 (s, 3H), 2.75-2.56 (m, 4H), 2.38-2.31 (m, 1H), 2.25 (s, 3H), 2.23-2.16 (m, 1H), 2.13-2.05 (m, 2H), 1.84-1.69 (m, 7H). LC-MS (long, acidic method) rt 1.620 min, 599.2 [M+H]

### 2-chloro-5-(1-methylpyrrolidin-3-yl)oxy-pyrimidine

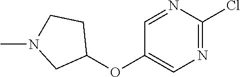

1-methylpyrrolidin-3-ol (0.27 mL, 2.47 mmol) was stirred in THF (3 mL). 2-chloropyrimidin-5-ol (0.65 g, 4.94 mmol), Triphenylphosphine (1.3 g, 4.94 mmol) and Di-tert-butylazodicarboxylate (1138.21 mg, 4.94 mmol) was added and the reaction allowed to stir at room temperature for 2 hours. The reaction mixture was diluted with ethyl acetate. 1N HCl (aq) was added. The layers were separated and the aqueous basified to pH 12 using 2N NaOH (aq). The aqueous was extracted with ethyl acetate (×3) dried (NaSO) and the solvent removed at reduced pressure to afford product 1H NMR (400 MHz, CDCl3)—8.24 (s, 2H), 4.89-4.80 (m, 1H), 2.95-2.85 (m, 2H), 2.78 (dd, J=10.9, 5.6 Hz, 1H), 2.49-2.36 (m, 5H), 2.03-1.95 (m, 1H). Product: 2-chloro-5-(1-methylpyrrolidin-3-yl)oxy-pyrimidine (525 mg, 2.4572 mmol, 99.417% yield)

### N-[5-[4-[[4-[2-(4-methylpiperazin-1-yl)ethoxy]pyrimidin-2-yl]amino]cyclohexoxy]-7-morpholino-1,6-naphthyridin-3-yl]methanesulfonamide (LFA2026)

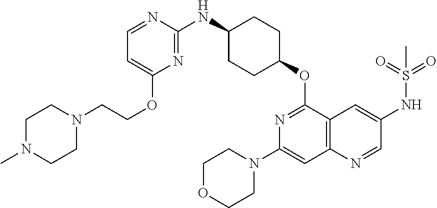 [SMILES: CN1CCN(CCOc2ccnc(N[C@H]3CC[C@@H](Oc4nc(N5CCOCC5)cc5ncc(NS(C)(=O)=O)cc45)CC3)n2)CC1]

NaH (10.8 mg, 0.27 mmol) was transferred to a 5 mL reaction flask, suspended in anhydrous THF (0.5 mL) and stirred with ice bath cooling under an argon atmosphere. 2-(4-methylpiperazin-1-yl)ethanol (37.3 mg, 0.26 mmol) dissolved in THF (0.5 mL) was added dropwise over 10 mins and the mixture stirred with cooling for 30 mins and then at RT for 30 mins. The mixture was cooled back again in an ice bath and a solution of N-[5-[4-[(4-chloropyrimidin-2-yl)amino]cyclohexoxy]-7-morpholino-1,6-naphthyridin-3-yl]methanesulfonamide (55.3 mg, 0.10 mmol) dissolved in THF (1 mL) was added over 10 mins. The mixture was stirred with cooling for an additional 30 mins and then the cooling bath removed. The reaction mixture was stirred at room temperature for 24 h and then quenched by addition of water (˜0.5 mL) and concentrated to dryness. Residue purified by basic prep HPLC (3×400 uL injections in DMSO) and relevant fractions were combined, concentrated in-vacuo and dried in a vacuum oven at 40° C. N-[5-[4-[[4-[2-(4-methylpiperazin-1-yl)ethoxy]pyrimidin-2-yl]amino]cyclohexoxy]-7-morpholino-1,6-naphthyridin-3-yl]methanesulfonamide LFA2026 (9.8 mg, 15% yield) was collected as a yellow solid.

1H NMR (400 MHz, DMSO)—8.68 (d, J=2.6 Hz, 1H), 8.11 (dd, J=2.7, 0.8 Hz, 1H), 7.99 (d, J=5.6 Hz, 1H), 6.99 (s, 1H), 6.53 (d, J=0.9 Hz, 1H), 5.97 (d, J=5.6 Hz, 1H), 5.36-5.30 (m, 1H), 4.38-4.31 (m, 2H), 3.90-3.78 (m, 1H), 3.77-3.71 (m, 4H), 3.50-3.43 (m, 4H), 3.01 (s, 3H), 2.63 (t, J=6.0 Hz, 2H), 2.49-2.20 (m, 8H), 2.13 (s, 3H), 2.13-2.07 (m, 2H), 1.87-1.67 (m, 6H).

LC-MS (long, acidic method) rt 1.40 min 642.3 [M+H]+, 99% @254 nm

### N-[5-[4-[(4-chloropyrimidin-2-yl)amino]cyclohexoxy]-7-morpholino-1,6-naphthyridin-3-yl]methanesulfonamide

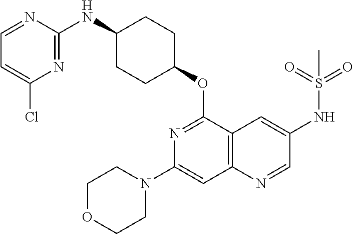 [SMILES: CS(=O)(=O)Nc1cnc2cc(N3CCOCC3)nc(O[C@H]3CC[C@@H](Nc4nccc(Cl)n4)CC3)c2c1]

Reference: WO2010/151747, 2010

N-[5-(4-aminocyclohexoxy)-7-morpholino-1,6-naphthyridin-3-yl]methanesulfonamide LFA021 (250 mg, 0.59 mmol) and 2,4-dichloropyrimidine (97.2 mg, 0.65 mmol) were transferred to a 5 mL microwave vial and ethanol (5 mL) added followed by triethylamine (165 uL, 1.19 mmol). The vial was capped and the mixture was heated in a microwave at 100° C. for 1 h. Mixture concentrated to dryness under reduced pressure and residue partitioned between water (20 mL) and DCM (2×20 mL). Combined organics dried (hydrophobic frit) and concentrated to dryness under reduced pressure. Residue purified by loading onto a 10 g silica column in DCM and eluting with MeOH (0-10% in DCM. Two peaks collected (tight separation). Peak 1 (minor regioisomer—desired product) collected as a orange gum, yield 55 mg.

1H NMR (400 MHz, DMSO)—9.89 (s, 1H), 8.70 (d, J=2.6 Hz, 1H), 8.24 (d, J=5.2 Hz, 1H), 8.13 (dd, J=2.7, 0.8 Hz, 1H), 7.73 (d, J=7.4 Hz, 1H), 6.65 (d, J=5.1 Hz, 1H), 6.54 (d, J=1.0 Hz, 1H), 5.36-5.30 (m, 1H), 3.91-3.78 (m, 1H), 3.77-3.68 (m, 4H), 3.51-3.44 (m, 4H), 3.04 (s, 3H), 2.17-2.01 (m, 2H), 1.87-1.62 (m, 6H).

LC-MS (long, acidic method) rt 2.36 min 534/536 [M+H]+, 76% @254 nm

Peak 2 collected as an orange gum (major isomer—product regioisomer), yield 111 mg.

1H NMR (400 MHz, DMSO)—9.89 (s, 1H), 8.70 (d, J=2.6 Hz, 1H), 8.12 (dd, J=2.7, 0.8 Hz, 1H), 7.96 (d, J=7.6 Hz, 1H), 7.89 (d, 1H), 6.54 (d, J=1.0 Hz, 1H), 6.49 (d, 1H), 5.35-5.30 (m, 1H), 4.04-3.89 (m, 1H), 3.78-3.71 (m, 4H), 3.51-3.44 (m, 4H), 3.03 (s, 3H), 2.13-2.01 (m, 2H), 1.92-1.76 (m, 4H), 1.75-1.62 (m, 2H).

LC-MS (long, acidic method) rt 2.17 min 534/536 [M+H]+, 100% @254 nm

### N-[5-[4-[[5-(2-aminoethoxy)pyrimidin-2-yl]amino]cyclohexoxy]-7-morpholino-1,6-naphthyridin-3-yl]methanesulfonamide (LFA2027)

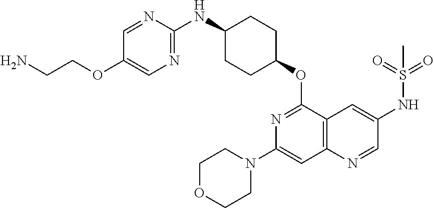 [SMILES: CS(=O)(=O)Nc1cnc2cc(N3CCOCC3)nc(O[C@H]3CC[C@@H](Nc4ncc(OCCN)cn4)CC3)c2c1]

Hydrochloric acid (3.7-4.3 M in 1,4-dioxane) (0.21 mL, 0.85 mmol) was added to a stirred solution of tert-butyl N-[2-[2-[[4-[[3-(methanesulfonamido)-7-morpholino-1,6-naphthyridin-5-yl]oxy]cyclohexyl]amino]pyrimidin-5-yl]oxyethyl]carbamate (56 mg, 0.09 mmol) in MeOH (2 mL). The mixture was stirred at RT for 22 h. The solvent was removed in vacuo and residue was dissolved in MeOH (1.5 mL) and was loaded directly onto an SCX column (1 g). The column was flushed with MeOH (100 mL) followed by 2 M NH₃/MeOH (50 mL). The NH₃/MeOH fraction was concentrated in vacuo to give the crude product (42 mg) as a yellow gum/residue. The crude product was purified basic prep. HPLC (method: basic prep.; 5-95% 0.005 M NHOH/MeCN in 0.005 M NH₄OH/HO) to give the product, N-[5-[4-[[5-(2-aminoethoxy)pyrimidin-2-yl]amino]cyclohexoxy]-7-morpholino-1,6-naphthyridin-3-yl]methanesulfonamide LFA2027 (33 mg, 69% yield), as a yellow solid.

1 h NMR (400 MHz, DMSO-d6)—8.63 (d, J=2.6 Hz, 1H), 8.12 (s, 2H), 8.03 (dd, J=2.6, 0.7 Hz, 1H), 6.73 (d, J=7.4 Hz, 1H), 6.50 (d, J=0.9 Hz, 1H), 5.33 (s, 1H), 3.92 (t, J=5.6 Hz, 2H), 3.82-3.66 (m, 5H), 3.45 (dd, J=6.4, 3.5 Hz, 4H), 2.95 (s, 3H), 2.88 (t, J=5.6 Hz, 2H), 2.17-2.01 (m, 2H), 1.90-1.64 (m, 6H). LCMS (long, acidic): 1.54 min, 559.2 [M+H]; 97% purity @254 nm.

### tert-butyl N-[2-[2-[[4-[[3-(methanesulfonamido)-7-morpholino-1,6-naphthyridin-5-yl]oxy]cyclohexyl]amino]pyrimidin-5-yl]oxyethyl]carbamate

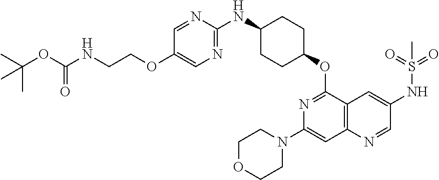 [SMILES: CC(C)(C)OC(=O)NCCOc1cnc(N[C@H]2CC[C@@H](Oc3nc(N4CCOCC4)cc4ncc(NS(C)(=O)=O)cc34)CC2)nc1]

A 2-5 mL microwave vial was charged with N-[5-(4-aminocyclohexoxy)-7-morpholino-1,6-naphthyridin-3-yl]methanesulfonamide (100 mg, 0.24 mmol), potassium tert-butoxide (133.1 mg, 1.19 mmol), BrettPhos Pd G3 (43.01 mg, 0.0500 mmol) and tert-butyl N-[2-(2-chloropyrimidin-5-yl)oxyethyl] carbamate (162 mg, 0.59 mmol). The vial was sealed and the mixture was purged with a stream of argon for 5 min. 1,4-dioxane (2.4 mL) was added and the mixture was degassed by sparging with argon for 5 min. The mixtures were heated to 90° C. and stirred for 18 h. After cooling to RT, further portions of tert-butyl N-[2-(2-chloropyrimidin-5-yl)oxyethyl] carbamate (64.9 mg, 0.24 mmol), potassium tert-butoxide (26.6 mg, 0.24 mmol) and BrettPhos Pd G3 (10.8 mg, 0.01 mmol) were added and the mixture was degassed by sparging with argon for 5 min. The mixture was re-heated to 90-C and stirred for 6 h. The reaction mixture was diluted with EtOAc (2 mL) and filtered through a short pad of Celite, eluting with EtOAc (50 mL). The filtrate was concentrated in vacuo to give an green/black gum (190 mg). The crude product was purified by flash column chromatography (25 g silica column, eluting with 0-10% MeOH in DCM) to give impure product as a yellow/orange gum (96 mg). The impure product was re-purified by flash column chromatography (20 g silica column, eluting with 0-5% MeOH in EtOAc) to give the product, tert-butylN-[2-[2-[[4-[[3-(methanesulfonamido)-7-morpholino-1,6-naphthyridin-5-yl]oxy]cyclohexyl]amino]pyrimidin-5-yl]oxyethyl]carbamate (56 mg 36% yield), as a yellow/orange gum.

1H NMR (400 MHz, Methanol-d4)—8.64 (d, J=2.7 Hz, 1H), 8.32 (dd, J=2.7, 0.8 Hz, 1H), 8.09 (s, 2H), 6.47 (d, J=0.9 Hz, 1H), 5.47-5.41 (m, 1H), 3.99 (t, J=5.5 Hz, 2H), 3.90-3.76 (m, 5H), 3.59-3.51 (m, 4H), 3.38 (t, J=5.5 Hz, 2H), 3.03 (s, 3H), 2.28-2.11 (m, 2H), 1.97-1.73 (m, 6H), 1.44 (s, 9H).

LCMS (short, acidic): 1.69 min, 659.2 [M+H]; 95% purity @254 nm.

### tert-butyl N-[2-(2-chloropyrimidin-5-yl)oxyethyl]carbamate

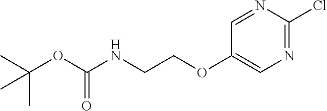 [SMILES: CC(C)(C)OC(=O)NCCOc1cnc(Cl)nc1]

2-chloropyrimidin-5-ol (200 mg, 1.53 mmol) and triphenylphosphine (804 mg, 3.06 mmol) were added to a stirred solution of tert-butyl N-(2-hydroxyethyl)carbamate (0.36 mL, 2.3 mmol) in THF (8 mL). The mixture was ice-cooled before di-tert-butylazodicarboxylate (394 mg, 1.71 mmol) was added in a single portion. The mixture was allowed to warm to RT and stirred for 20 h. The mixture was concentrated in vacuo and the residue was purified by flash column chromatography (25 g silica column, eluting with 0-80% EtOAc in n-heptane) to give an off-white solid (310 mg). 1H NMR was consistent with the product structure at 78% purity (22 wt % pyrimidinol SM present).

The solid was re-purified by flash column chromatography (20 g silica column, eluting with 0-5% MeOH in DCM) to give the product, tert-butyl N-[2-(2-chloropyrimidin-5-yl)oxyethyl] carbamate (221 mg, 0.807 mmol, 53% yield), as a white solid.

1H NMR (400 MHz, DMSO-d6)—8.53 (s, 2H), 7.04 (t, J=5.6 Hz, 1H), 4.15 (t, J=5.6 Hz, 2H), 3.30 (q, J=5.9 Hz, 2H), 1.37 (s, 9H).

LCMS (short, acidic): 1.66 min, 218.1/220.2 [M-Bu+H]; 97% purity @254 nm.

### N-[5-[4-[[5-(2-hydroxyethoxy)pyrimidin-2-yl]amino]cyclohexoxy]-7-morpholino-1,6-naphthyridin-3-yl]methanesulfonamide (LFA2028)

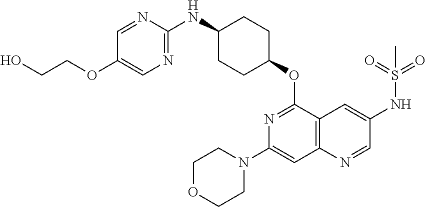 [SMILES: CS(=O)(=O)Nc1cnc2cc(N3CCOCC3)nc(O[C@H]3CC[C@@H](Nc4ncc(OCCO)cn4)CC3)c2c1]

N-[5-[4-[[5-[2-[tert-butyl(dimethyl)silyl]oxyethoxy]pyrimidin-2-yl]amino]cyclohexoxy]-7-morpholino-1,6-naphthyridin-3-yl]methanesulfonamide (42 mg, 0.06 mmol) was stirred in THF (1 mL). Tetrabutylammonium fluoride in THF (0.09 mL, 0.09 mmol) was added and the reaction stirred for 1 hour at room temperature. Further tetrabutylammonium fluoride in THF (0.09 mL, 0.09 mmol) was added and the reaction stirred for 3 hours. The solvent was removed at reduced pressure. The residue was purified by basic prep HPLC. The appropriate fractions were combined and the solvent removed at reduced pressure. Residue found to contain TBAF related impurities. The residue was taken up in DCM and washed with water. The organic was dried (Na₂SO₄) and the solvent removed at reduced pressure. Further purification by flash chromatography (5 g SiO) eluting with 0-5% methanol in DCM followed by loading onto a 1 g SCX. The column was washed with methanol and eluted with 2M ammonia in methanol. The ammonia fraction was concentrated at reduced pressure to afford LFA2028 (17.3 mg, 50% yield)

1H NMR (400 MHz, DMSO)—9.86 (s, 1H), 8.70 (d, J=2.6 Hz, 1H), 8.20-8.01 (m, 3H), 6.71 (d, J=7.4 Hz, 1H), 6.53 (d, J=0.9 Hz, 1H), 5.40-5.29 (m, 1H), 4.85 (t, J=5.5 Hz, 1H), 3.95 (dd, J=5.4, 4.5 Hz, 2H), 3.88-3.72 (m, 5H), 3.66 (td, J=5.4, 4.5 Hz, 2H), 3.48 (t, J=4.9 Hz, 4H), 3.03 (s, 3H), 2.16-2.01 (m, 2H), 1.93-1.57 (m, 6H).

LC-MS (long, acidic method) rt 1.858 min, 560.2 [M+H]+ Product

### N-[5-[4-[[5-[2-[tert-butyl(dimethyl)silyl]oxyethoxy]pyrimidin-2-yl]amino]cyclohexoxy]-7-morpholino-1,6-naphthyridin-3-yl]methanesulfonamide

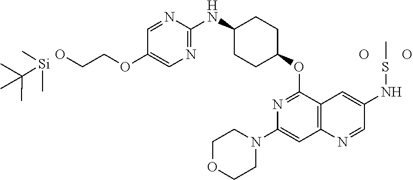

A 20 mL microwave vials was charged with N-[5-(4-aminocyclohexoxy)-7-morpholino-1,6-naphthyridin-3-yl]methanesulfonamide (150.mg, 0.3600 mmol), tert-butyl-[2-(2-chloropyrimidin-5-yl)oxyethoxy]-dimethyl-silane(154.19 mg, 0.5300 mmol), Potassium tert-butoxide (119.79 mg, 1.07 mmol) and tBuBrettPhos Pd G3 (30.41 mg, 0.0400 mmol). The vial was sealed and the mixture was purged with a stream of argon for 5 min. 1,4-Dioxane (7 mL) was added and the mixture was degassed by sparging with argon for 5 min. The mixture was heated to 100-C with stirring for 5 h. After cooling to RT the reaction mixture was filtered through Dicalite, eluting with DCM and MeOH. The filtrate was concentrated in vacuo. The resulting residue was purified by flash chromatography (20 g SiO) eluting with a gradient 0-10% methanol in DCM. The appropriate fractions were combined and the solvent removed at reduced pressure to afford product

1H NMR (400 MHz, DMSO)—9.88 (s, 1H), 8.70 (d, J=2.6 Hz, 1H), 8.13 (dd, J=2.7, 0.8 Hz, 1H), 8.10 (s, 2H), 6.73 (d, J=7.4 Hz, 1H), 6.54 (d, J=0.9 Hz, 1H), 5.36-5.24 (m, 1H), 4.01 (dd, 2H), 3.86 (dd, J=5.4, 3.9 Hz, 2H), 3.78-3.70 (m, 5H), 3.51-3.43 (m, 4H), 3.03 (s, 3H), 2.13-2.05 (m, 2H), 1.83-1.68 (m, 6H), 0.86 (s, 9H), 0.05 (s, 6H). LC-MS (long, acidic method) rt 2.993 min, 674.2 [M+H]+

### tert-butyl-[2-(2-chloropyrimidin-5-yl)oxyethoxy]-dimethyl-silane

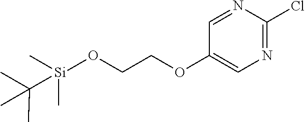 [SMILES: CC(C)(C)[Si](C)(C)OCCOc1cnc(Cl)nc1]

2-[tert-butyl(dimethyl)silyl]oxyethanol (1013 mg, 5.75 mmol) and Triphenylphosphine (2009 mg, 7.66 mmol) were added to a stirred solution of 2-chloropyrimidin-5-ol (500 mg, 3.83 mmol) in THF (19 mL). The mixture was ice-cooled before Di-tert-butylazodicarboxylate (1323.03 mg, 5.75 mmol) was added in a single portion. The mixture was allowed to warm to RT and stirred for 17 h. The mixture was concentrated in vacuo and the residue was purified by flash column chromatography (50 g silica column, eluting with 0-20% EtOAc in n-heptane) to give product as a mobile oil which solidified on standing to give a white solid. 1H NMR (400 MHz, CDCl3)—8.24 (s, 2H), 4.07 (dd, J=5.3, 4.2 Hz, 2H), 3.90 (dd, J=5.3, 4.1 Hz, 2H), 0.81 (s, 9H), 0.00 (s, 6H). LC-MS (long, acidic method) rt 3.068 min; [M+H+] 289.2. Product: tert-butyl-[2-(2-chloropyrimidin-5-yl)oxyethoxy]-dimethyl-silane (1.292 g, 4.4729 mmol, 116.77% yield) Used without further purification in subsequent reactions

### N-[7-morpholino-5-[4-[(5-morpholinopyrimidin-2-yl)amino]cyclohexoxy]-1,6-naphthyridin-3-yl]methanesulfonamide (LFA2029)

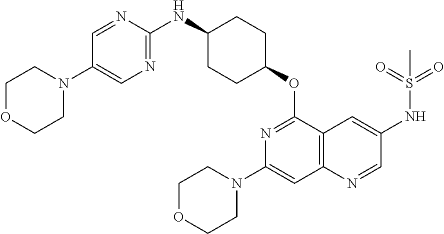 [SMILES: CS(=O)(=O)Nc1cnc2cc(N3CCOCC3)nc(O[C@H]3CC[C@@H](Nc4ncc(N5CCOCC5)cn4)CC3)c2c1]

N-[5-[4-[(5-bromopyrimidin-2-yl)amino]cyclohexoxy]-7-morpholino-1,6-naphthyridin-3-yl]methanesulfonamide (100 mg, 0.17 mmol), sodium tert-butoxide (49.8 mg, 0.52 mmol), JohnPhos (5.2 mg, 0.02 mmol), tris(dibenzylideneacetone)dipalladium(0) (15.8 mg, 0.02 mmol) and morpholine (29.8 uL, 0.35 mmol) in 1,4-Dioxane (3.5 mL) were combined in a microwave vial. The vial was sealed and argon bubbled through the reaction mixture for 5 minutes. The reaction mixture was heated to 100° C. for 17 h and then cooled to room temperature. Further morpholine (0.03 mL, 0.35 mmol), JohnPhos (5.2 mg, 0.02 mmol) and tris(dibenzylideneacetone)dipalladium(0) (15.8 mg, 0.02 mmol) were added, the vial capped and argon bubbled through the mixture for 5 mins. The mixture was heated at 100° C. for an additional 4 h and then cooled to room temperature, diluted with DCM, filtered through celite and washed with DCM and then MeOH. Filtrate and washings were combined and the residue purified by loading onto a 10 g silica cartridge in DCM and eluting with 2M ammonia in MeOH (0-10%) in DCM. Relevant fractions were combined and concentrated to give an orange gum. Further purified by basic prep HPLC (5×200 uL injections in DMSO). Relevant fractions were combined and concentrated to give 46 mg of a yellow solid—LC-MS showed a mixture of product+H-product. Further purified by acidic prep HPLC (4×200 uL injections in DMSO). Relevant fractions were combined to give after concentration in-vacuo N-[7-morpholino-5-[4-[(5-morpholinopyrimidin-2-yl)amino]cyclohexoxy]-1,6-naphthyridin-3-yl]methanesulfonamide LFA2029 (17.7 mg, 18% yield) as a pale orange solid.

1H NMR (400 MHz, DMSO)—9.84 (s, 1H), 8.70 (d, J=2.6 Hz, 1H), 8.13 (dd, J=2.6, 0.8 Hz, 1H), 8.10 (s, 2H), 6.63 (d, J=7.4 Hz, 1H), 6.54 (d, J=0.9 Hz, 1H), 5.37-5.30 (m, 1H), 3.84-3.66 (m, 9H), 3.52-3.43 (m, 4H), 3.04 (s, 3H), 2.98-2.91 (m, 4H), 2.15-2.04 (m, 2H), 1.85-1.65 (m, 6H).

LCMS (long, acidic): 1.96 min, 585.2 [M+H]+ 98.1% purity @ 254 nm

### N-[5-[4-[(5-bromopyrimidin-2-yl)amino]cyclohexoxy]-7-morpholino-1,6-naphthyridin-3-yl]methanesulfonamide

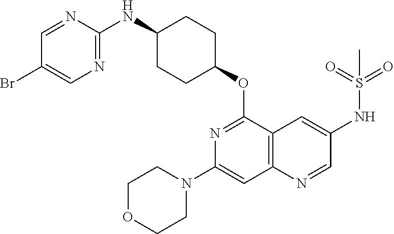 [SMILES: CS(=O)(=O)Nc1cnc2cc(N3CCOCC3)nc(O[C@H]3CC[C@@H](Nc4ncc(Br)cn4)CC3)c2c1]

Triethylamine (0.3 mL, 2.14 mmol) was added to a stirred solution of N-[5-(4-aminocyclohexoxy)-7-morpholino-1,6-naphthyridin-3-yl]methanesulfonamide LFA021 (300 mg, 0.71 mmol) and 5-bromo-2-chloro-pyrimidine (151 mg, 0.78 mmol) in EtOH (5 mL). The reaction was heated to 90° C. for 17 hours. Further 5-bromo-2-chloro-pyrimidine (68.8 mg, 0.36 mmol) was added and the heating continued for 24 hours. The mixture was concentrated in vacuo and the residue was partitioned between DCM and water. The aqueous phase was extracted with DCM (2×25 mL). The combined organic phases were dried (phase-separator) and concentrated in vacuo. The resulting residue was purified by flash chromatography (20 g SiO2) eluting with a gradient 0-5% 2M ammonia in methanol in DCM. The appropriate fractions were combined and the solvent removed at reduced pressure to afford product

1H NMR (400 MHz, CDCl3)—8.72 (d, J=2.7 Hz, 1H), 8.32-8.25 (m, 3H), 6.54 (d, J=0.9 Hz, 1H), 5.47-5.37 (m, 2H), 3.98-3.91 (m, 1H), 3.89-3.82 (m, 4H), 3.59-3.52 (m, 4H), 3.08 (s, 3H), 2.21-2.13 (m, 2H), 2.01-1.93 (m, 2H), 1.90-1.74 (m, 5H).

LCMS (long, acidic): 2.388 min, 578.1/580.1 [M+H]+

### N-[7-morpholino-5-[4-[[5-(2-morpholinoethyl)pyrimidin-2-yl]amino]cyclohexoxy]-1,6-naphthyridin-3-yl]methanesulfonamide (LFA2030)

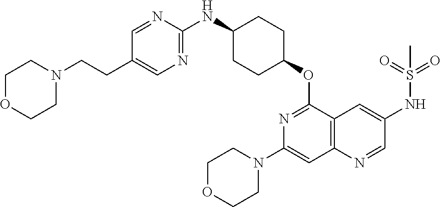 [SMILES: CS(=O)(=O)Nc1cnc2cc(N3CCOCC3)nc(O[C@H]3CC[C@@H](Nc4ncc(CCN5CCOCC5)cn4)CC3)c2c1]

Morpholine (0.03 mL, 0.33 mmol) was added to a stirred solution of N-[7-morpholino-5-[4-[[5-(2-oxoethyl)pyrimidin-2-yl]amino]cyclohexoxy]-1,6-naphthyridin-3-yl]methanesulfonamide (120 mg, 0.22 mmol) in DCM (2 mL). The mixture was stirred at RT for 10 min. before sodium triacetoxyborohydride (93.9 mg, 0.44 mmol) was added. The mixture was stirred at RT for 72 h. The mixture was diluted with DCM (20 mL) and washed with sat. NaHCO (aq.) (20 mL). The aqueous phase was extracted with DCM (2×20 mL). The combined organic phases were dried (phase-separator) and concentrated in vacuo to give the crude product as an orange/brown gum (112 mg). The crude product was purified by flash column chromatography (10 g silica column, eluting with 0-10% MeOH in DCM) to give the a yellow/orange gum (37 mg), still containing some impurities. The yellow/orange gum was further purified by basic prep. HPLC (method: basic prep.; 5-95% 0.005 M NH₄OH/MeCN in 0.005 M NH₄OH/HO) to give a yellow solid (24 mg), still containing some small impurities. The yellow solid was further purified by acidic prep. HPLC (method: acidic prep.; 5-95% 0.1% HCOOH/MeCN in 0.1% HCOOH/H₂O) to give a red solid (42 mg). The red solid was dissolved in MeOH (12 mL) and loaded onto an SCX column (1 g). The column was flushed with MeOH (100 mL) followed by 2 M NH/MeOH (50 mL). The NH₃/MeOH fraction was concentrated in vacuo to give the product, N-[7-morpholino-5-[4-[[5-(2-morpholinoethyl)pyrimidin-2-yl]amino]cyclohexoxy]-1,6-naphthyridin-3-yl]methanesulfonamide LFA2030 (19 mg, 14% yield), as a yellow solid.

1H NMR (400 MHz, Methanol-d4)—8.65 (d, J=2.7 Hz, 1H), 8.33 (dd, J=2.7, 0.8 Hz, 1H), 8.18 (s, 2H), 6.48 (d, J=0.9 Hz, 1H), 5.50-5.43 (m, 1H), 3.97-3.86 (m, 1H), 3.87-3.79 (m, 4H), 3.75-3.64 (m, 4H), 3.60-3.49 (m, 4H), 3.03 (s, 3H), 2.63 (dd, J=7.7, 7.2 Hz, 2H), 2.58-2.45 (m, 6H), 2.28-2.12 (m, 2H), 2.02-1.74 (m, 6H)

LCMS (long, acidic): 1.55 min, 613.3 [M+H]; 99% purity @254 nm

### N-[7-morpholino-5-[4-[[5-(2-oxoethyl)pyrimidin-2-yl]amino]cyclohexoxy]-1,6-naphthyridin-3-yl]methanesulfonamide

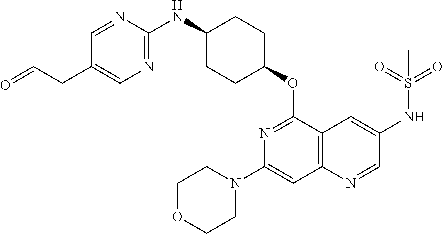 [SMILES: CS(=O)(=O)Nc1cnc2cc(N3CCOCC3)nc(O[C@H]3CC[C@@H](Nc4ncc(CC=O)cn4)CC3)c2c1]

2.5 M Hydrochloric acid (aq.) (1 mL, 2.5 mmol) was added to a stirred solution of N-[5-[4-[[5-[(E)-2-ethoxyvinyl]pyrimidin-2-yl]amino]cyclohexoxy]-7-morpholino-1,6-naphthyridin-3-yl]methanesulfonamide (228.mg, 0.3200 mmol) in THF (3 mL). The mixture was heated to 80° C. and stirred for 1 h. After cooling to RT, the mixture was basified with sat. NaHCO₃ (aq.) (30 mL) and extracted with DCM (3×30 mL). The combined organic phases were dried (phase-separator) and concentrated in vacuo to give the crude product as an orange/brown gum (202 mg). 1H NMR (400 MHz, Chloroform-) was consistent with the product structure at ca. 50% purity LCMS (short, acidic): 1.53 min, 542.2 [M+H]; again, product does not run well on LCMS. 80 mg of the orange/brown gum was purified by flash column chromatography (10 g silica column, eluting with 0-10% MeOH in DCM) to give the product, N-[7-morpholino-5-[4-[[5-(2-oxoethyl)pyrimidin-2-yl]amino]cyclohexoxy]-1,6-naphthyridin-3-yl]methanesulfonamide (36 mg, 0.0665 mmol, 20.759% yield), as a yellow gum/glass. 1H NMR (400 MHz, Chloroform-) was consistent with the product structure at 95% purity. 1d1H NMR (400 MHz, Chloroform-d)—9.76 (t, J=0.8 Hz, 1H), 8.74 (d, J=2.7 Hz, 1H), 8.26 (dd, J=2.6, 0.8 Hz, 1H), 8.14 (s, 2H), 6.52 (d, J=0.9 Hz, 1H), 5.70 (d, J=7.9 Hz, 1H), 5.50-5.41 (m, 1H), 4.05-3.93 (m, 1H), 3.85 (dd, J=5.8, 3.9 Hz, 4H), 3.58-3.50 (m, 6H), 3.09 (s, 3H), 2.24-2.09 (m, 2H), 2.03-1.90 (m, 2H), 1.91-1.68 (m, 4H). LCMS (short, acidic): 1.28-1.72 min, 542.2 [M+H];

### N-[5-[4-[[5-[2-(4-methylpiperazin-1-yl)ethyl]pyrimidin-2-yl]amino]cyclohexoxy]-7-morpholino-1,6-naphthyridin-3-yl]methanesulfonamide (LFA2031)

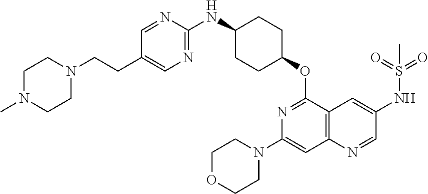 [SMILES: CN1CCN(CCc2cnc(N[C@H]3CC[C@@H](Oc4nc(N5CCOCC5)cc5ncc(NS(C)(=O)=O)cc45)CC3)nc2)CC1]

1-methylpiperazine (11.1 uL, 0.10 mmol) was added to a stirred solution of N-[7-morpholino-5-[4-[[5-(2-oxoethyl)pyrimidin-2-yl]amino]cyclohexoxy]-1,6-naphthyridin-3-yl]methanesulfonamide (36 mg, 0.07 mmol) in DCM (2 mL). The mixture was stirred at RT for 10 min. before sodium triacetoxyborohydride (28.2 mg, 0.13 mmol) was added. The mixture was stirred at RT for 20 h. The mixture was diluted with DCM (20 mL) and washed with sat. NaHCO₃ (aq.) (20 mL). The aqueous phase was extracted with DCM (2×20 mL). The combined organic phases were dried (phase-separator) and concentrated in vacuo to give the crude product as a dark yellow gum (37 mg). The crude product was purified by flash column chromatography (10 g silica column, eluting with 0-10% 2 M NH/MeOH in DCM) to give an orange gum (22 mg), still containing minor impurities. The orange gum was further purified by acidic prep. HPLC (method: acidic prep.; 5-95% 0.1% HCOOH/MeCN in 0.1% HCOOH/H₂O) to give a red solid (24 mg). The red solid was dissolved in MeOH and was loaded directly onto an SCX column (1 g). The column was flushed with MeOH (100 mL) followed by 2 M NH₃/MeOH (50 mL). The NH₃/MeOH fraction was concentrated in vacuo to give the product, N-[5-[4-[[5-[2-(4-methylpiperazin-1-yl)ethyl]pyrimidin-2-yl]amino]cyclohexoxy]-7-morpholino-1,6-naphthyridin-3-yl]methanesulfonamide LFA2031 (13.8 mg, 33% yield), as a yellow solid.

1H NMR (400 MHz, Methanol-d4)—8.64 (d, J=2.7 Hz, 1H), 8.33 (dd, J=2.6, 0.8 Hz, 1H), 8.18 (s, 2H), 6.48 (d, J=1.0 Hz, 1H), 5.50-5.43 (m, 1H), 3.95-3.87 (m, 1H), 3.85-3.79 (m, 4H), 3.60-3.52 (m, 4H), 3.02 (s, 3H), 2.79-2.41 (m, 12H), 2.32 (s, 3H), 2.25-2.15 (m, 2H), 2.01-1.75 (m, 6H).

LCMS (long, acidic): 1.52 min, 626.3 [M+H]; 99% purity @254 nm.

### N-[5-[4-[[5-(4-methylpiperazin-1-yl)pyrimidin-2-yl]amino]cyclohexoxy]-7-morpholino-1,6-naphthyridin-3-yl]methanesulfonamide (LFA2032)

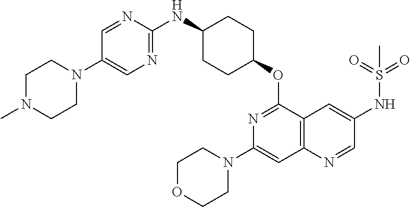 [SMILES: CN1CCN(c2cnc(N[C@H]3CC[C@@H](Oc4nc(N5CCOCC5)cc5ncc(NS(C)(=O)=O)cc45)CC3)nc2)CC1]

N-[5-[4-[(5-bromopyrimidin-2-yl)amino]cyclohexoxy]-7-morpholino-1,6-naphthyridin-3-yl]methanesulfonamide (25 mg, 0.04 mmol), 1-methylpiperazine (19.2 uL, 0.17 mmol), sodium tert-butoxide (24.9 mg, 0.26 mmol), JohnPhos (2.58 mg, 0.010 mmol) and tris(dibenzylideneacetone)dipalladium(0) (7.9 mg, 0.010 mmol), 1,4-dioxane (1 mL) were combined in in a 0.5-2 mL microwave vial. The vial was sealed and argon bubbled through the reaction mixture for 5 minutes. The reaction mixture was heated to 100° C. for 18 hours. The reaction was diluted with ethyl acetate and filtered through a dicalite pad and the pad was washed with ethyl acetate. The combined filtrate was concentrated at reduced pressure. The resulting residue was purified by basic prep HPLC. The appropriate fractions were combined and the solvent removed at reduced pressure to afford product (15.3 mg, 30% yield)

1H NMR (400 MHz, CDCl3)—8.74 (d, J=2.6 Hz, 1H), 8.24 (dd, J=2.7, 0.8 Hz, 1H), 8.09 (s, 2H), 6.52 (s, 1H), 5.44 (s, 1H), 5.30 (d, J=8.0 Hz, 1H), 3.98-3.90 (m, 1H), 3.89-3.82 (m, 4H), 3.54 (dd, J=5.8, 4.0 Hz, 4H), 3.08 (s, 3H), 3.07-3.02 (m, 4H), 2.64-2.55 (m, 4H), 2.37 (s, 3H), 2.19-2.11 (m, 2H), 2.01-1.92 (m, 2H), 1.89-1.69 (m, 5H).

LCMS (long, acidic): 1.554 min, 598.2 [M+H]+

### 5-[4-[[5-(1-methylpyrrolidin-3-yl)oxypyrimidin-2-yl]amino]cyclohexoxy]-7-morpholino-1,6-naphthyridin-3-ol (LFA2033)

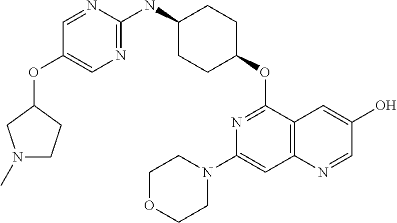 [SMILES: CN1CCC(Oc2cnc(N[C@H]3CC[C@@H](Oc4nc(N5CCOCC5)cc5ncc(O)cc45)CC3)nc2)C1]

A 2-5 mL microwave vials were charged with 5-(4-aminocyclohexoxy)-7-morpholino-1,6-naphthyridin-3-ol (LFA112) (100 mg, 0.29 mmol), 2-chloro-5-(1-methylpyrrolidin-3-yl)oxy-pyrimidine (93.05 mg, 0.4400 mmol), Potassium tert-butoxide (97.74 mg, 0.8700 mmol) and tBuBrettPhos Pd G3 (24.81 mg, 0.0300 mmol). The vial was sealed and the mixture was purged with a stream of argon for 5 min. 1,4-Dioxane (5 mL) was added and the mixture was degassed by sparging with argon for 5 min. The mixture was heated to 100° C. with stirring for 5 h. After cooling to RT the reaction mixture was filtered through a short pad of Celite, eluting with DCM (20 mL) and then MeOH (10 mL). The filtrate was concentrated in vacuo to give a brown gum. Purified by loading onto a 10 g silica column in DCM eluting with 2M ammonia in MeOH (0-10%) in DCM. Relevant fractions were combined to afford 5-[4-[[5-(1-methylpyrrolidin-3-yl)oxypyrimidin-2-yl]amino]cyclohexoxy]-7-morpholino-1,6-naphthyridin-3-ol (91 mg, 60% yield) as a pale orange solid.

1H NMR (400 MHz, DMSO) δ:10.00 (s, 1H), 8.53 (d, J=2.9 Hz, 1H), 8.05 (s, 2H), 7.62 (dd, J=2.9, 0.8 Hz, 1H), 6.74 (d, J=7.6 Hz, 1H), 6.48 (d, J=0.9 Hz, 1H), 5.34 (s, 1H), 4.79-4.70 (m, 1H), 3.81-3.70 (m, 5H), 3.42-3.36 (m, 4H), 2.75-2.54 (m, 3H), 2.38-2.28 (m, 1H), 2.24 (s, 3H), 2.23-2.15 (m, 1H), 2.13-2.02 (m, 2H), 1.85-1.67 (m, 7H).

LC-MS (long, acidic method) rt 1.49 min, 522.2 [M+H]+

30 mg further purified by basic prep HPLC (2×300 UL injections in DMSO) (method: basic prep.; 5-95% 0.005 M NH₄OH/MeCN in 0.005 M NH₄OH/H2O). Relevant fractions were combined and concentrated in-vacuo to afford 5-[4-[[5-(1-methylpyrrolidin-3-yl)oxypyrimidin-2-yl]amino]cyclohexoxy]-7-morpholino-1,6-naphthyridin-3-ol (18.4 mg, 12% yield) as a yellow solid.

1H NMR (400 MHz, DMSO) δ: 10.03 (s, 1H), 8.53 (d, J=2.9 Hz, 1H), 8.05 (s, 2H), 7.62 (dd, J=2.9, 0.8 Hz, 1H), 6.74 (d, J=7.6 Hz, 1H), 6.48 (d, J=0.9 Hz, 1H), 5.36-5.31 (m, 1H), 4.79-4.70 (m, 1H), 3.81-3.70 (m, 5H), 3.42-3.36 (m, 4H), 2.74-2.52 (m, 3H), 2.38-2.28 (m, 1H), 2.24 (s, 3H), 2.23-2.15 (m, 1H), 2.13-2.01 (m, 2H), 1.85-1.67 (m, 7H).

LC-MS (long, acidic method) rt 1.49 min, 522.3 [M+H]+

### 5-[4-[[5-[2-(4-methylpiperazin-1-yl)ethoxy]pyrimidin-2-yl]amino]cyclohexoxy]-7-morpholino-1,6-naphthyridin-3-ol (LFA2034)

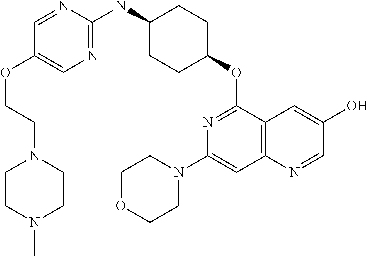 [SMILES: CN1CCN(CCOc2cnc(N[C@H]3CC[C@@H](Oc4nc(N5CCOCC5)cc5ncc(O)cc45)CC3)nc2)CC1]

A 2-5 mL microwave vials were charged with 5-(4-aminocyclohexoxy)-7-morpholino-1,6-naphthyridin-3-ol (100 mg, 0.29 mmol), 2-chloro-5-[2-(4-methylpiperazin-1-yl)ethoxy]pyrimidine (111.8 mg, 0.44 mmol), Potassium tert-butoxide (97.74 mg, 0.87 mmol) and tBuBrettPhos Pd G3 (24.81 mg, 0.030 mmol). The vial was sealed and the mixture was purged with a stream of argon for 5 min. 1,4-Dioxane (5 mL) was added and the mixture was degassed by sparging with argon for 5 min. The mixture was heated to 100° C. with stirring for 5 h. After cooling to RT the reaction mixture was filtered through a short pad of Celite, eluting with DCM (20 mL) and then MeOH (10 mL). The filtrate was concentrated in vacuo to give a brown gum. Purified by loading onto a 10 g silica column in DCM eluting with 2M ammonia in MeOH (0-10%) in DCM. Relevant fractions were combined to afford 5-[4-[[5-[2-(4-methylpiperazin-1-yl)ethoxy]pyrimidin-2-yl]amino]cyclohexoxy]-7-morpholino-1,6-naphthyridin-3-ol (107 mg, 65% yield) as a pale orange solid.

1H NMR (400 MHz, DMSO) δ: 9.99 (s, 1H), 8.53 (d, J=2.9 Hz, 1H), 8.11 (s, 2H), 7.62 (dd, J=3.0, 0.8 Hz, 1H), 6.73 (d, J=7.6 Hz, 1H), 6.48 (d, J=0.8 Hz, 1H), 5.37-5.31 (m, 1H), 4.03 (t, J=5.8 Hz, 2H), 3.81-3.68 (m, 5H), 3.43-3.36 (m, 4H), 2.62 (t, J=5.8 Hz, 2H), 2.47-2.20 (m, 8H), 2.14 (s, 3H), 2.11-2.01 (m, 2H), 1.83-1.68 (m, 6H).

LC-MS (long, acidic method) rt 1.51 min, 565.2 [M+H]+

30 mg was further purified by basic prep HPLC (2×300 UL injections in DMSO) (method: basic prep.; 5-95% 0.005 M NH₄OH/MeCN in 0.005 M NH₄OH/H2O). 30 mg of further relevant fractions were combined and concentrated in-vacuo to afford 5-[4-[[5-[2-(4-methylpiperazin-1-yl)ethoxy]pyrimidin-2-yl]amino]cyclohexoxy]-7-morpholino-1,6-naphthyridin-3-ol (16.4 mg, 10% yield) as a yellow solid.

1H NMR (400 MHz, DMSO) δ: 10.03 (s, 1H), 8.53 (d, J=2.9 Hz, 1H), 8.11 (s, 2H), 7.61 (dd, J=3.0, 0.8 Hz, 1H), 6.73 (d, J=7.7 Hz, 1H), 6.48 (d, J=0.9 Hz, 1H), 5.36-5.31 (m, 1H), 4.03 (t, J=5.8 Hz, 2H), 3.80-3.70 (m, 5H), 3.42-3.36 (m, 4H), 2.62 (t, J=5.8 Hz, 2H), 2.49-2.19 (m, 8H), 2.13 (s, 3H), 2.11-2.02 (m, 2H), 1.83-1.68 (m, 6H).

LC-MS (long, acidic method) rt 1.47 min, 565.3 [M+H]+

### 7-morpholino-5-[4-[[5-(2-morpholinoethoxy)pyrimidin-2-yl]amino]cyclohexoxy]-1,6-naphthyridin-3-ol (LFA2035)

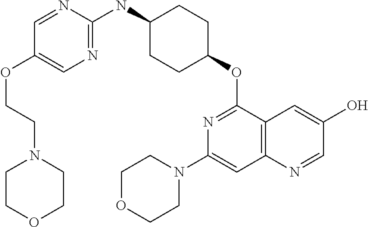 [SMILES: Oc1cnc2cc(N3CCOCC3)nc(O[C@H]3CC[C@@H](Nc4ncc(OCCN5CCOCC5)cn4)CC3)c2c1]

A 2-5 mL microwave vials were charged with 4-[2-(2-chloropyrimidin-5-yl)oxyethyl]morpholine (106.1 mg, 0.44 mmol), 5-(4-aminocyclohexoxy)-7-morpholino-1,6-naphthyridin-3-ol (100 mg, 0.29 mmol), Potassium tert-butoxide (97.7 mg, 0.87 mmol) and tBuBrettPhos Pd G3 (24.8 mg, 0.03 mmol). The vial was sealed and the mixture was purged with a stream of argon for 5 min. 1,4-Dioxane (5 mL) was added and the mixture was degassed by sparging with argon for 5 min. The mixture was heated to 100-C with stirring for 3 h. After cooling to RT the reaction mixture was filtered through a short pad of Celite, eluting with DCM (20 mL) and then MeOH (10 mL). The filtrate was concentrated in vacuo to give a brown gum. Purified by SCX (2×2 g columns) by loading on in MeOH+AcOH, washing with MeOH and eluting product off in 2M ammonia in MeOH to afford a brown gum, yield 212 mg. Further purified by loading onto a 10 g silica column in DCM eluting with MeOH (0-10%) in DCM. Fractions containing the second major eluting product were combined and concentrated in-vacuo to afford 7-morpholino-5-[4-[[5-(2-morpholinoethoxy)pyrimidin-2-yl]amino]cyclohexoxy]-1,6-naphthyridin-3-ol (113 mg, 71% yield) as a yellow gum.

NMR and LC-MS consistent with the crude desired product. COSY experiment shows an interaction of NH and C—H of cyclohexyl (under morpholine signal).

1H NMR (400 MHz, DMSO) δ: 10.00 (s, 1H), 8.53 (d, J=2.9 Hz, 1H), 8.11 (s, 2H), 7.62 (dd, J=2.9, 0.8 Hz, 1H), 6.73 (d, J=7.6 Hz, 1H), 6.48 (d, J=0.8 Hz, 1H), 5.36-5.31 (m, 1H), 4.05 (t, J=5.7 Hz, 2H), 3.82-3.68 (m, 5H), 3.59-3.54 (m, 4H), 3.42-3.36 (m, 4H), 2.63 (t, J=5.7 Hz, 2H), 2.47-2.41 (m, 4H), 2.14-2.02 (m, 2H), 1.83-1.67 (m, 6H). LC-MS (long, acidic method) rt 1.48 min, 552.2 [M+H]+

30 mg further purified by basic prep HPLC (2×300 UL injections in DMSO) (method: basic prep.; 5-95% 0.005 M NH₄OH/MeCN in 0.005 M NH₄OH/H2O). Fractions containing second eluting product were combined and concentrated in-vacuo to afford 7-morpholino-5-[4-[[5-(2-morpholinoethoxy)pyrimidin-2-yl]amino]cyclohexoxy]-1,6-naphthyridin-3-ol (18.6 mg, 12% yield) as a yellow solid.

1H NMR (400 MHz, DMSO) δ: 10.07 (s, 1H), 8.53 (d, J=2.9 Hz, 1H), 8.11 (s, 2H), 7.61 (dd, J=2.9, 0.8 Hz, 1H), 6.74 (d, J=7.7 Hz, 1H), 6.47 (d, J=0.9 Hz, 1H), 5.36-5.31 (m, 1H), 4.05 (t, J=5.7 Hz, 2H), 3.81-3.70 (m, 5H), 3.60-3.53 (m, 4H), 3.42-3.36 (m, 4H), 2.63 (t, J=5.7 Hz, 2H), 2.47-2.41 (m, 4H), 2.15-2.02 (m, 2H), 1.85-1.67 (m, 6H).

LC-MS (long, acidic method) rt 1.52 min, 552.3 [M+H]+

N-[5-[4-[[5-[(3S)-1-methylpyrrolidin-3-yl]oxypyrimidin-2-yl]amino]cyclohexoxy]-7-morpholino-1,6-naphthyridin-3-yl] methanesulfonamide (LFA2036)

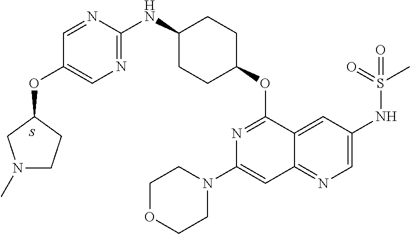

N-[5-(4-aminocyclohexoxy)-7-morpholino-1,6-naphthyridin-3-yl]methanesulfonamide; hydrochloride (0.05 g, 0.109 mmol), 2-chloro-5-[(3S)-1-methylpyrrolidin-3-yl]oxy-pyrimidine (0.023 g, 0.109 mmol), BrettPhos Pd G3 (0.01042 g, 0.01092 mmol), BrettPhos (0.006041 g, 0.01092 mmol), 1,4-dioxane (1.032 g, 1 mL, 11.71 mmol) and potassium tert-butoxide (1 mol/L) in THF (0.3 g, 0.3275 mL, 0.3275 mmol) were combined in a microwave vial, seal and heated at 100° C. for 3 hours. The reaction mixture was cooled, filtered through a single frit column and concentrated. The residue was purified via reverse phase chromatography (MeCN, pH10 aq) to give 14 mg of a yellow solid, which contained minor impurities and was submitted for prep HPLC. Compound returned at pH10 as a yellow solid, N-[5-[4-[[5-[(3S)-1-methylpyrrolidin-3-yl]oxypyrimidin-2-yl]amino]cyclohexoxy]-7-morpholino-1,6-naphthyridin-3-yl]methanesulfonamide (0.014 g, 21%).

1H NMR (500 MHz, CHLOROFORM-d) δ: 8.73 (d, J=2.29 Hz, 1H), 8.27 (d, J=2.29 Hz, 1H), 7.99-8.02 (m, 2H), 6.48-6.57 (m, 1H), 6.02-6.13 (m, 1H), 5.39-5.51 (m, 1H), 4.63-4.71 (m, 1H), 3.91-3.97 (m, 1H), 3.83-3.88 (m, 4H), 3.52-3.59 (m, 4H), 3.03 (s, 3H), 2.98-3.01 (m, 1H), 2.95 (d, J=11.46 Hz, 1H), 2.60-2.64 (m, 1H), 2.44 (s, 3H), 2.33-2.40 (m, 1H), 2.24-2.31 (m, 1H), 2.18 (dd, J=4.01, 9.16 Hz, 2H), 1.93-2.01 (m, 3H), 1.72-1.88 (m, 5H). LCMS (MeCN, pH10): 1.00 min, 599 [M+H]+

### 2-chloro-5-[(3S)-1-methylpyrrolidin-3-yl]oxy-pyrimidine

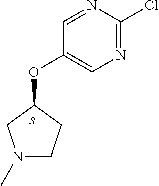

To a solution of 2-chloropyrimidin-5-ol (0.1 g, 0.76611 mmol) and (3R)-1-methylpyrrolidin-3-ol (0.085241 g, 0.84272 mmol) in THF (4.43 g, 5 mL, 61.4 mmol) was added triphenylphosphine (0.30141 g, 0.2694 mL, 1.1492 mmol), followed by dropwise addition of diisopropylazodicarboxylate (0.18590 g, 0.182 mL, 0.91933 mmol). The reaction mixture was left to stir overnight. The reaction mixture was concentrated and purified via reverse phase column chromatography (MeCN, pH10 aq) to give an orange gum, 2-chloro-5-[(3S)-1-methylpyrrolidin-3-yl]oxy-pyrimidine (0.121 g, 74%).

1H NMR (500 MHz, CHLOROFORM-d) 8.22 (s, 2H), 4.81-4.86 (m, 1H), 2.82-2.89 (m, 2H), 2.74-2.79 (m, 1H), 2.42 (dt, J=6.59, 8.45 Hz, 1H), 2.38 (s, 3H), 2.32-2.37 (m, 1H), 1.91-1.99 (m, 1H)

LCMS (MeCN, pH10): 1.00 min, 214.2 [M+H]+

### N-[5-[4-[[5-[(3R)-1-methylpyrrolidin-3-yl] oxypyrimidin-2-yl]amino]cyclohexoxy]-7-morpholino-1,6-naphthyridin-3-yl] methanesulfonamide (LFA2037)

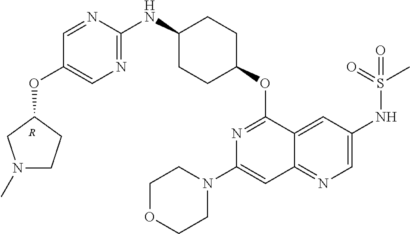

N-[5-(4-aminocyclohexoxy)-7-morpholino-1,6-naphthyridin-3-yl]methanesulfonamide; hydrochloride (0.05 g, 0.1092 mmol), BrettPhos Pd G3 (0.01042 g, 0.01092 mmol), BrettPhos (0.006041 g, 0.01092 mmol), 1,4-dioxane (1.032 g, 1 mL, 11.71 mmol) and potassium tert-butoxide (1 mol/L) in THF (0.3 g, 0.3275 mL, 0.3275 mmol) were combined in a microwave vial, sealed and heated at 100° C. for 3 hours. The reaction mixture was cooled, filtered through a single frit column and concentrated. The residue was purified via reverse phase chromatography (MeCN, pH10 aq) to give 14 mg of a yellow solid, which contained minor impurities and was submitted for prep HPLC. Compound returned at pH10 as yellow solid, N-[5-[4-[[5-[(3R)-1-methylpyrrolidin-3-yl]oxypyrimidin-2-yl]amino]cyclohexoxy]-7-morpholino-1,6-naphthyridin-3-yl]methanesulfonamide (0.0077 g, 12%)

1H NMR (500 MHz, CHLOROFORM-d) δ: 8.72-8.75 (m, 1H), 8.26-8.28 (m, 1H), 8.02 (s, 2H), 6.51-6.55 (m, 1H), 5.84-5.91 (m, 1H), 5.42-5.47 (m, 1H), 4.69-4.74 (m, 1H), 3.89-3.95 (m, 1H), 3.84-3.88 (m, 4H), 3.53-3.57 (m, 4H), 3.04 (s, 3H), 2.96-3.02 (m, 2H), 2.58 (br. s., 1H), 2.52 (s, 3H), 2.26-2.34 (m, 1H), 2.15-2.21 (m, 2H), 1.99-2.05 (m, 1H), 1.92-1.98 (m, 2H), 1.73-1.87 (m, 4H)

LCMS (MeOH, pH1): 1.47 min, 599.3 [M+H]+

### 2-chloro-5-[(3R)-1-methylpyrrolidin-3-yl]oxy-pyrimidine

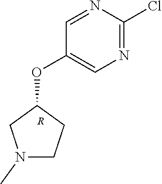

To a solution of 2-chloropyrimidin-5-ol (0.3 g, 2.2983 mmol) and (3S)-1-methylpyrrolidin-3-ol (0.25572 g, 2.5282 mmol) in THF (4.43 g, 5 mL, 61.4 mmol) was added triphenylphosphine (0.90424 g, 0.8081 mL, 3.4475 mmol), followed by dropwise addition of DIISOPROPYL AZODICARBOXYLATE (0.55769 g, 0.547 mL, 2.7580 mmol). The reaction mixture was left to stir overnight. The reaction mixture was concentrated and purified via reverse phase column chromatography (MeCN, pH10 aq) to give an orange gum, 2-chloro-5-[(3R)-1-methylpyrrolidin-3-yl]oxy-pyrimidine (0.362 g, 52%).

¹H NMR (500 MHz, CHLOROFORM-d) δ: 8.23-8.28 (m, 2H), 4.94-5.02 (m, 1H), 4.86-4.92 (m, 1H), 2.87-2.98 (m, 3H), 2.53-2.60 (m, 1H), 2.48 (s, 3H), 2.37-2.44 (m, 1H), 2.00-2.08 (m, 1H)

LCMS (MeCN, pH10): 0.78 min, 214.2 [M+H]+

### N-[5-[4-[[4-methyl-5-(4-methylpiperazin-1-yl)pyrimidin-2-yl]amino]cyclohexoxy]-7-morpholino-1,6-naphthyridin-3-yl]methanesulfonamide (LFA2038)

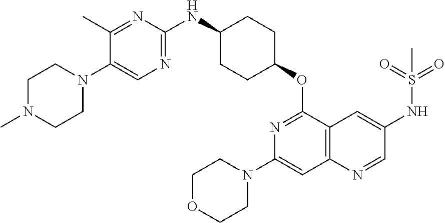 [SMILES: Cc1nc(N[C@H]2CC[C@@H](Oc3nc(N4CCOCC4)cc4ncc(NS(C)(=O)=O)cc34)CC2)ncc1N1CCN(C)CC1]

A microwave vial was charged with N-[5-[4-[(5-chloro-4-methyl-pyrimidin-2-yl)amino]cyclohexoxy]-7-morpholino-1,6-naphthyridin-3-yl]methanesulfonamide (77 mg, 0.1405 mmol, 100 mass %), 1-methylpiperazine (F, 4.3 equiv., 0.6041 mmol, 100 mass %), sodium tert-butoxide (6.5 equiv., 0.9132 mmol, 100 mass %), tris(dibenzylideneacetone)dipalladium(0) (0.25 equiv., 0.03512 mmol, 100 mass %) and 2-(di-tert-butylphosphino)biphenyl (0.25 equiv., 0.03512 mmol, 99 mass %). The vial was capped and placed under an atmosphere of nitrogen. Degassed 1,4-dioxane (1 ml, 11.71 mmol, 100 mass %) was added and the reaction heated thermally at 100° C. overnight. Solvents were evaporated and the crude mixture submitted for purification by preparative HPLC to give N-[5-[4-[[4-methyl-5-(4-methylpiperazin-1-yl)pyrimidin-2-yl]amino]cyclohexoxy]-7-morpholino-1,6-naphthyridin-3-yl]methanesulfonamide (1.5 mg, 0.0023 mmol, 1.7%) as the final product.

1H NMR (500 MHz, CHLOROFORM-d) δ 8.73-8.78 (m, 1H), 8.21-8.28 (m, 1H), 8.02-8.09 (m, 1H), 6.51-6.56 (m, 1H), 5.40-5.45 (m, 1H), 5.15-5.30 (m, 1H), 3.91-4.00 (m, 1H), 3.83-3.89 (m, 4H), 3.52-3.58 (m, 4H), 3.08 (s, 3H), 2.89-2.97 (m, 4H), 2.52-2.74 (m, 4H), 2.38-2.45 (m, 3H), 2.35 (s, 3H), 2.12-2.19 (m, 2H), 1.93-1.98 (m, 2H), 1.79-1.88 (m, 4H).

LCMS: Rt=1.84 min., no mass ion (MeOH, pH10)

### N-[5-[4-[(5-chloro-4-methyl-pyrimidin-2-yl)amino]cyclohexoxy]-7-morpholino-1,6-naphthyridin-3-yl]methanesulfonamide

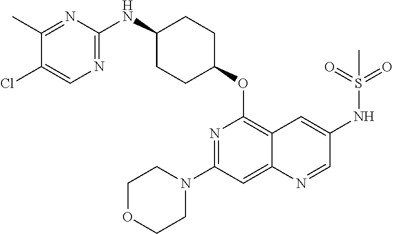 [SMILES: Cc1nc(N[C@H]2CC[C@@H](Oc3nc(N4CCOCC4)cc4ncc(NS(C)(=O)=O)cc34)CC2)ncc1Cl]

N-[5-(4-aminocyclohexoxy)-7-morpholino-1,6-naphthyridin-3 yl]methanesulfonamide; hydrochloride (200 mg, 0.4367 mmol), 2,5-dichloro-4-methyl-pyrimidine (2.5 equiv., 1.092 mmol) and 2,5-dichloro-4-methyl-pyrimidine (2.5 equiv., 1.092 mmol) were combined in ethanol (0.5 mL, 9 mmol, 0.5 mL) in a microwave vial and the reaction heated thermally at 100° C. overnight. Ethanol was evaporated. Purification by column chromatography using Biotage Isolera One (SiO₂, gradient from 1:9 ethyl acetate/petroleum ether to 100% ethyl acetate), followed by 1% methanol/ethyl acetate to 20% methanol/ethyl acetate), gave the product N-[5-[4-[(5-chloro-4-methyl-pyrimidin-2-yl)amino]cyclohexoxy]-7-morpholino-1,6-naphthyridin-3-yl]methanesulfonamide (112 mg, 0.2043 mmol, 46.79%) as a brown solid.

1H NMR (500 MHz, DMSO-d6) δ 9.89-9.93 (m, 1H), 8.69 (d, J=2.29 Hz, 1H), 8.20 (s, 1H), 7.33-7.40 (m, 1H), 6.54 (s, 1H), 5.33-5.36 (m, 1H), 5.31-5.34 (m, 1H), 5.29-5.33 (m, 1H), 3.77-3.84 (m, 1H), 3.72-3.75 (m, 4H), 3.45-3.49 (m, 4H), 3.03 (s, 3H), 2.33 (s, 3H), 2.05-2.15 (m, 2H), 1.69-1.83 (m, 6H).

Rt=1.50 min., m/z at 549 MH+(MeCN, pH1).

### N-[5-[4-[[4-methyl-5-[2-(4-methylpiperazin-1-yl)ethoxy]pyrimidin-2-yl]amino]cyclohexoxy]-7-morpholino-1,6-naphthyridin-3-yl]methanesulfonamide (LFA2039)

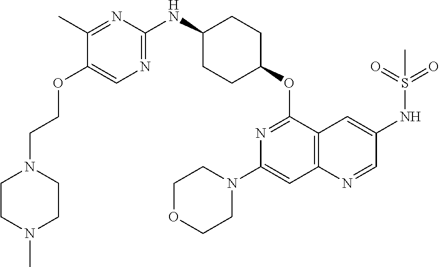 [SMILES: Cc1nc(N[C@H]2CC[C@@H](Oc3nc(N4CCOCC4)cc4ncc(NS(C)(=O)=O)cc34)CC2)ncc1OCCN1CCN(C)CC1]

Prepared using a method analogous to Buchwald-Hartwig reaction used for LFA2001 to afford the titled compound (11 mg, 8%).

1H NMR (500 MHz, CHLOROFORM-d) δ 8.74-8.79 (m, 1H), 8.20-8.26 (m, 1H), 7.89 (s, 1H), 6.49-6.55 (m, 1H), 5.39-5.46 (m, 1H), 5.12-5.26 (m, 1H), 3.98-4.05 (m, 2H), 3.89-3.95 (m, 1H), 3.83-3.88 (m, 4H), 3.51-3.57 (m, 4H), 3.08 (s, 3H), 2.77-2.80 (m, 2H), 2.43-2.76 (m, 8H), 2.35 (br. s., 3H), 2.31 (s, 3H), 2.10-2.17 (m, 2H), 1.95 (dd, J=3.15, 8.88 Hz, 2H), 1.71-1.87 (m, 4H).

LCMS: Rt=0.96 min., m/z at 656 MH+(MeCN, pH10)

### N-[5-[4-[[4-methyl-5-(2-morpholinoethoxy)pyrimidin-2-yl]amino]cyclohexoxy]-7-morpholino-1,6-naphthyridin-3-yl]methanesulfonamide (LFA2040)

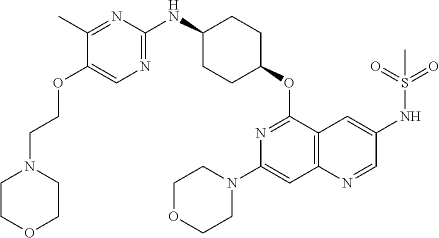 [SMILES: Cc1nc(N[C@H]2CC[C@@H](Oc3nc(N4CCOCC4)cc4ncc(NS(C)(=O)=O)cc34)CC2)ncc1OCCN1CCOCC1]

Prepared using a method analogous to Buchwald-Hartwig reaction used for LFA2001 to afford the titled compound (11 mg, 8%).

1H NMR (500 MHz, DMSO-d6) δ 8.69 (d, J=2.86 Hz, 1H), 8.11 (d, J=2.86 Hz, 1H), 7.98-8.01 (m, 1H), 6.61 (d, J=7.45 Hz, 1H), 6.53 (s, 1H), 5.30-5.34 (m, 1H), 4.01 (t, J=5.73 Hz, 2H), 3.72-3.75 (m, 4H), 3.55-3.58 (m, 4H), 3.45-3.49 (m, 4H), 3.02 (s, 3H), 2.62-2.65 (m, 2H), 2.43-2.48 (m, 4H), 2.21 (s, 3H), 2.03-2.11 (m, 2H), 1.67-1.82 (m, 6H).

LCMS: Rt=1.1 min., m/z at 643 MH+(MeCN, pH1).

### 2-chloro-4-methyl-5-[2-(4-methylpiperazin-1-yl)ethoxy]pyrimidine

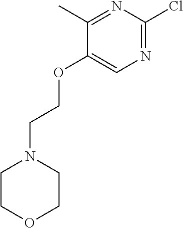 [SMILES: Cc1nc(Cl)ncc1OCCN1CCOCC1]

To an RBF was added 2-chloro-4-methyl-pyrimidin-5-ol (0.1 g, 0.69175 mmol), 2-(4-methylpiperazin-1-yl)ethanol (0.12969 g, 0.89928 mmol), triphenylphosphine (0.23587 g, 0.2108 mL, 0.89928 mmol) and diisopropyl azodicarboxylate (0.18184 g, 0.178 mL, 0.89928 mmol) in THF (17.7 g, 20 mL, 246 mmol) at r.t. Mixture stirred at this temperature overnight. Concentrated and purified by reverse phase column chromatography (Biotage Isolera Four, gradient from 1:9 methanol/water pH10 to 100% methanol) to give the product 2-chloro-4-methyl-5-[2-(4-methylpiperazin-1-yl)ethoxy]pyrimidine (150 mg, 0.55400 mmol, 80%) as a white solid.

1H NMR (500 MHz, CHLOROFORM-d) δ 8.08 (s, 1H), 6.35 (br. s., 2H), 4.18 (t, J=5.73 Hz, 2H), 2.86 (t, J=5.44 Hz, 2H), 2.56-2.70 (m, 4H), 2.47 (s, 3H), 2.30 (s, 3H).

LCMS: Rt=0.5 min, m/z at 271 MH+(MeCN, pH1).

### General Procedure for Alkylation of Sulfonamide or Phenol

To a vial was added Sulfonamide/Phenol (1.0 eq.), 5-(chloromethyl)-1-methyl-2-nitro-imidazole or 5-(1-chloroethyl)-1-methyl-2-nitro-imidazole (1.5 equiv.), potassium carbonate (2 equiv.) and N,N-dimethylformamide (0.5 ml) the mixture was stirred at room temperature for 18 hours. −2 drops of distilled water were added to fully dissolve inorganics and the mixture purified by preparative HPLC to afford the desired product.

### 4-[3-[1-(3-methyl-2-nitro-imidazol-4-yl)ethoxy]-5-(4-pyrazin-2-ylcyclohexoxy)-1,6-naphthyridin-7-yl]morpholine (LFA2041)

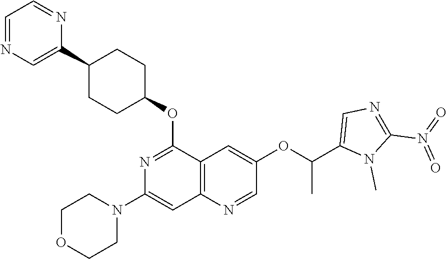 [SMILES: CC(Oc1cnc2cc(N3CCOCC3)nc(O[C@H]3CC[C@@H](c4cnccn4)CC3)c2c1)c1cnc([N+](=O)[O-])n1C]

Prepared using analogous method to general condition for phenol alkylation using 5-(1-chloroethyl)-1-methyl-2-nitro-imidazole and 7-morpholino-5-(4-pyrazin-2-ylcyclohexoxy)-1,6-naphthyridin-3-ol to afford title compound (7.2 mg, 0.012 mmol, 95 mass %, 25% Yield) as a yellow solid.

1H NMR (500 MHz, CHLOROFORM-d) δ 8.59-8.61 (m, 1H), 8.52-8.54 (m, 1H), 8.48-8.51 (m, 1H), 8.42-8.44 (m, 1H), 7.78-7.81 (m, 1H), 7.31-7.35 (m, 1H), 6.51-6.54 (m, 1H), 5.57-5.65 (m, 2H), 4.06-4.11 (m, 3H), 3.84-3.90 (m, 4H), 3.48-3.52 (m, 4H), 2.88-2.97 (m, 1H), 2.26-2.35 (m, 2H), 2.05-2.17 (m, 2H), 1.76-1.90 (m, 7H) LCMS pH1/MeCN RT=1.410 minutes, 95.4% (UV 254 nm), [M+H]+: 561.2

### 4-[3-[(3-methyl-2-nitro-imidazol-4-yl)methoxy]-5-(4-pyrazin-2-ylcyclohexoxy)-1,6-naphthyridin-7-yl]morpholine (LFA2042)

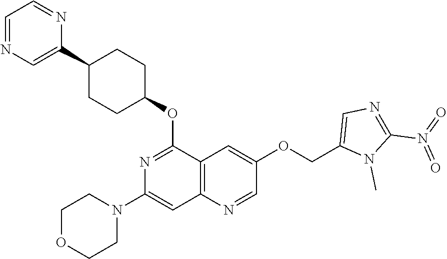 [SMILES: Cn1c(COc2cnc3cc(N4CCOCC4)nc(O[C@H]4CC[C@@H](c5cnccn5)CC4)c3c2)cnc1[N+](=O)[O-]]

Prepared using analogous method to general condition for phenol alkylation using 5-(chloromethyl)-1-methyl-2-nitro-imidazole and 7-morpholino-5-(4-pyrazin-2-ylcyclohexoxy)-1,6-naphthyridin-3-ol to afford title compound (7.8 mg, 0.014 mmol, 95 mass %, 28% Yield) as a yellow solid.

1H NMR (500 MHz, CHLOROFORM-d) δ 8.59-8.63 (m, 1H), 8.52-8.55 (m, 1H), 8.49-8.51 (m, 1H), 8.41-8.44 (m, 1H), 7.81-7.86 (m, 1H), 7.38-7.43 (m, 1H), 6.53-6.57 (m, 1H), 5.57-5.63 (m, 1H), 5.21-5.26 (m, 2H), 4.11-4.15 (m, 3H), 3.84-3.89 (m, 4H), 3.48-3.54 (m, 4H), 2.90-2.99 (m, 1H), 2.30-2.39 (m, 2H), 2.10-2.22 (m, 2H), 1.79-1.94 (m, 4H) LCMS pH1/MeCN RT=1.382 minutes, 91.3% (UV 254 nm), [M+H]+: 547.2

### N-[1-(3-methyl-2-nitro-imidazol-4-yl)ethyl]-N-[7-morpholino-5-(4-pyrazin-2-ylcyclohexoxy)-1,6-naphthyridin-3-yl]methanesulfonamide (LFA2043)

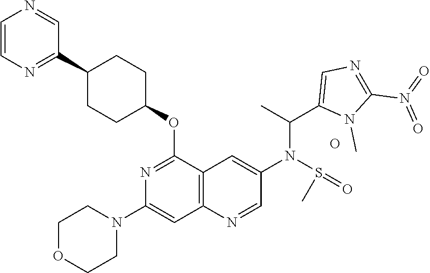

Prepared using analogous method to general condition for sulfonamide alkylation using 5-(1-chloroethyl)-1-methyl-2-nitro-imidazole and N-[7-morpholino-5-(4-pyrazin-2-ylcyclohexoxy)-1,6-naphthyridin-3-yl]methanesulfonamide to afford title compound (5.3 mg, 0.0079 mmol, 95 mass %, 49% Yield) as a yellow solid.

1H NMR (500 MHz, CHLOROFORM-d) δ 8.50-8.52 (m, 1H), 8.48-8.50 (m, 1H), 8.42-8.44 (m, 1H), 8.31-8.33 (m, 1H), 7.90-7.94 (m, 1H), 6.64-6.69 (m, 1H), 6.42-6.46 (m, 1H), 5.77-5.85 (m, 1H), 5.49-5.56 (m, 1H), 4.22-4.27 (m, 3H), 3.81-3.85 (m, 4H), 3.55-3.62 (m, 4H), 3.06-3.11 (m, 3H), 2.86-2.96 (m, 1H), 2.14-2.33 (m, 2H), 1.96-2.09 (m, 2H), 1.82-1.92 (m, 2H), 1.78-1.81 (m, 5H) LCMS pH1/MeCN RT=1.361 minutes, 92.5% (UV 254 nm), [M+H]+: 638.2

### N-[5-[4-[[5-[4-(2-fluoroethyl)piperazin-1-yl]pyrimidin-2-yl]amino]cyclohexoxy]-7-morpholino-1,6-naphthyridin-3-yl]-N-[(3-methyl-2-nitro-imidazol-4-yl)methyl]methanesulfonamide (LFA2044)

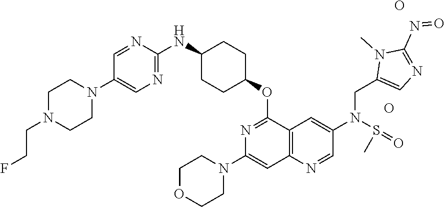

Prepared using analogous method to general condition for sulfonamide alkylation using 5-(chloromethyl)-1-methyl-2-nitro-imidazole and N-[5-[4-[[5-[4-(2-fluoroethyl)piperazin-1-yl]pyrimidin-2-yl]amino]cyclohexoxy]-7-morpholino-1,6-naphthyridin-3-yl]methanesulfonamide to afford title compound (9.6 mg, 0.012 mmol, 95 mass %, 59% Yield) as a yellow solid.

1H NMR (500 MHz, CHLOROFORM-d) δ 8.57-8.61 (m, 1H), 8.07-8.11 (m, 2H), 8.02-8.04 (m, 1H), 6.85-6.87 (m, 1H), 6.44-6.47 (m, 1H), 5.28-5.42 (m, 2H), 4.98-5.03 (m, 2H), 4.50-4.68 (m, 2H), 4.07-4.13 (m, 3H), 3.87-4.00 (m, 1H), 3.77-3.84 (m, 4H), 3.53-3.58 (m, 4H), 3.00-3.08 (m, 7H), 2.69-2.84 (m, 6H), 2.07-2.13 (m, 2H), 1.94-2.00 (m, 2H), 1.82-1.90 (m, 2H), 1.67-1.75 (m, 2H) LCMS pH1/MeCN RT=1.151 minutes, 98.9% (UV 254 nm), [M+H]+: 769.3

### N-[(3-methyl-2-nitro-imidazol-4-yl)methyl]-N-[7-morpholino-5-[4-[[5-[4-(2,2,2-trifluoroethyl)piperazin-1-yl]pyrimidin-2-yl]amino]cyclohexoxy]-1,6-naphthyridin-3-yl]methanesulfonamide (LFA2045)

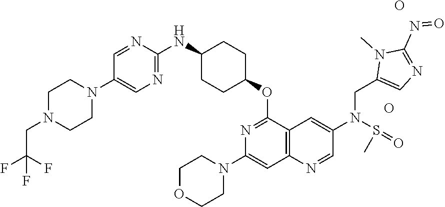

Prepared using analogous method to general condition for sulfonamide alkylation using 5-(chloromethyl)-1-methyl-2-nitro-imidazole and N-[7-morpholino-5-[4-[[5-[4-(2,2,2-trifluoroethyl)piperazin-1-yl]pyrimidin-2-yl]amino]cyclohexoxy]-1,6-naphthyridin-3-yl]methanesulfonamide to afford title compound (8.6 mg, 0.010 mmol, 95 mass %, 52% Yield) as a yellow solid.

1H NMR (500 MHz, CHLOROFORM-d) δ 8.57-8.62 (m, 1H), 8.06-8.10 (m, 2H), 8.00-8.03 (m, 1H), 6.84-6.87 (m, 1H), 6.44-6.48 (m, 1H), 5.32-5.40 (m, 1H), 5.10-5.20 (m, 1H), 4.97-5.02 (m, 2H), 4.07-4.14 (m, 3H), 3.90-3.99 (m, 1H), 3.80-3.85 (m, 4H), 3.52-3.58 (m, 4H), 2.99-3.07 (m, 9H), 2.79-2.87 (m, 4H), 2.06-2.14 (m, 2H), 1.92-2.00 (m, 2H), 1.80-1.91 (m, 2H), 1.69-1.74 (m, 2H) LCMS pH1/MeCN RT=1.491 minutes, 95.4% (UV 254 nm), [M+H]+: 805.2

### N-[1-(3-methyl-2-nitro-imidazol-4-yl)ethyl]-N-[7-morpholino-5-[4-[[5-[4-(2,2,2-trifluoroethyl)piperazin-1-yl]pyrimidin-2-yl]amino]cyclohexoxy]-1,6-naphthyridin-3-yl]methanesulfonamide (LFA2046)

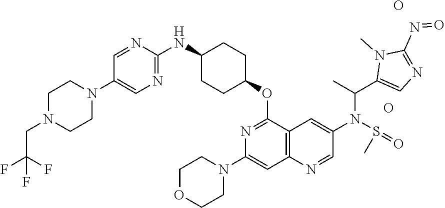

Prepared using analogous method to general condition for sulfonamide alkylation using 5-(1-chloroethyl)-1-methyl-2-nitro-imidazole and N-[7-morpholino-5-[4-[[5-[4-(2,2,2-trifluoroethyl)piperazin-1-yl]pyrimidin-2-yl]amino]cyclohexoxy]-1,6-naphthyridin-3-yl]methanesulfonamide to afford title compound (10.6 mg, 0.0123 mmol, 95 mass %, 63% Yield) as a yellow solid.

1H NMR (500 MHz, CHLOROFORM-d) δ 8.37-8.43 (m, 1H), 8.07-8.10 (m, 2H), 7.60-7.68 (m, 1H), 6.60-6.67 (m, 1H), 6.38-6.46 (m, 1H), 5.75-5.85 (m, 1H), 5.40-5.49 (m, 1H), 5.25-5.34 (m, 1H), 4.24-4.31 (m, 3H), 3.88-3.97 (m, 1H), 3.78-3.86 (m, 4H), 3.50-3.61 (m, 4H), 2.98-3.10 (m, 9H), 2.80-2.86 (m, 4H), 2.02-2.14 (m, 2H), 1.89-1.97 (m, 2H), 1.76-1.88 (m, 2H), 1.71-1.74 (m, 3H), 1.48-1.66 (m, 2H) LCMS pH10/MeCN RT=1.790 minutes, 97.0% (UV 254 nm), [M+H]+: 819.2

### N-[5-[4-[[5-[4-(2,2-difluoroethyl)piperazin-1-yl]pyrimidin-2-yl]amino]cyclohexoxy]-7-morpholino-1,6-naphthyridin-3-yl]-N-[1-(3-methyl-2-nitro-imidazol-4-yl)ethyl]methanesulfonamide (LFA2047)

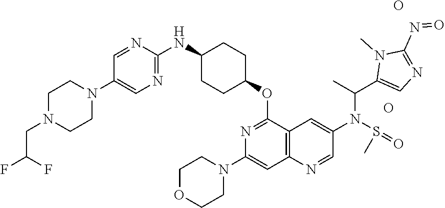

Prepared using analogous method to general condition for sulfonamide alkylation using 5-(1-chloroethyl)-1-methyl-2-nitro-imidazole and N-[5-[4-[[5-[4-(2,2-difluoroethyl)piperazin-1-yl]pyrimidin-2-yl]amino]cyclohexoxy]-7-morpholino-1,6-naphthyridin-3-yl]methanesulfonamide to afford title compound (6.6 mg, 0.0078 mmol, 95 mass %, 56% Yield) as a yellow solid.

1H NMR (500 MHz, CHLOROFORM-d) δ 8.39-8.43 (m, 1H), 8.07-8.10 (m, 2H), 7.64-7.67 (m, 1H), 6.60-6.67 (m, 1H), 6.42-6.45 (m, 1H), 5.76-6.04 (m, 2H), 5.41-5.46 (m, 1H), 5.25-5.34 (m, 1H), 4.26-4.31 (m, 3H), 3.88-3.98 (m, 1H), 3.80-3.85 (m, 4H), 3.51-3.63 (m, 4H), 3.05-3.08 (m, 3H), 3.00-3.04 (m, 4H), 2.77-2.85 (m, 2H), 2.73-2.77 (m, 5H), 2.03-2.15 (m, 2H), 1.89-1.99 (m, 2H), 1.76-1.88 (m, 2H), 1.71-1.75 (m, 3H), 1.43-1.63 (m, 2H) LCMS pH10/MeCN RT=1.648 minutes, 98.9% (UV 254 nm), [M+H]+: 801.3

### N-[5-[4-[[5-[4-(2,2-difluoroethyl)piperazin-1-yl]pyrimidin-2-yl]amino]cyclohexoxy]-7-morpholino-1,6-naphthyridin-3-yl]-N-[(3-methyl-2-nitro-imidazol-4-yl)methyl]methanesulfonamide (LFA2048)

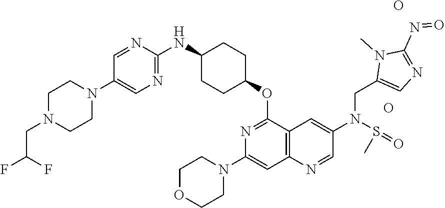

Prepared using analogous method to general condition for sulfonamide alkylation using 5-(chloromethyl)-1-methyl-2-nitro-imidazole and N-[5-[4-[[5-[4-(2,2-difluoroethyl)piperazin-1-yl]pyrimidin-2-yl]amino]cyclohexoxy]-7-morpholino-1,6-naphthyridin-3-yl]methanesulfonamide to afford title compound (4.7 mg, 0.0057 mmol, 95 mass %, 41% Yield) as a yellow solid.

1H NMR (500 MHz, CHLOROFORM-d) δ 8.57-8.62 (m, 1H), 8.06-8.09 (m, 2H), 8.00-8.03 (m, 1H), 6.85-6.87 (m, 1H), 6.44-6.47 (m, 1H), 5.76-6.04 (m, 1H), 5.33-5.40 (m, 1H), 5.12-5.19 (m, 1H), 4.95-5.04 (m, 2H), 4.08-4.15 (m, 3H), 3.89-3.99 (m, 1H), 3.79-3.86 (m, 4H), 3.53-3.58 (m, 4H), 3.00-3.08 (m, 7H), 2.71-2.84 (m, 6H), 2.05-2.14 (m, 2H), 1.82-1.99 (m, 4H), 1.68-1.73 (m, 2H) LCMS pH10/MeCN RT=1.614 minutes, 99.4% (UV 254 nm), [M+H]+: 787.3

### N-[4-[[3-[1-(3-methyl-2-nitro-imidazol-4-yl)ethoxy]-7-morpholino-1,6-naphthyridin-5-yl]oxy]cyclohexyl]-5-(2-morpholinoethoxy)pyrimidin-2-amine (LFA2049)

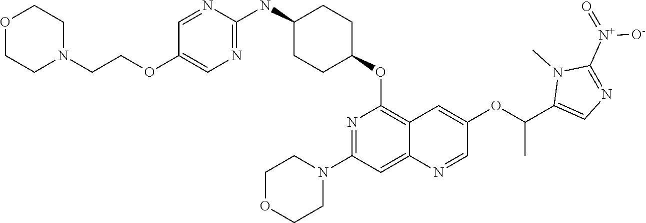 [SMILES: CC(Oc1cnc2cc(N3CCOCC3)nc(O[C@H]3CC[C@@H](Nc4ncc(OCCN5CCOCC5)cn4)CC3)c2c1)c1cnc([N+](=O)[O-])n1C]

7-morpholino-5-[4-[[5-(2-morpholinoethoxy)pyrimidin-2-yl]amino]cyclohexoxy]-1,6-naphthyridin-3-ol (30 mg, 0.05 mmol) and Potassium carbonate (22.55 mg, 0.16 mmol) were transferred to a 10 mL RB flask and stirred at RT under an argon atmosphere. 5-(1-chloroethyl)-1-methyl-2-nitro-imidazole (11.34 mg, 0.06 mmol) LFA009 dissolved in DMF (0.50 mL) was added in one portion and the suspension stirred at room temperature for 17 h (overnight). Suspension filtered through a syringe filter and washed through with DMSO. Combined filtrate and washings purified by basic prep HPLC (2 runs) and relevant fractions combined and concentrated to dryness in-vacuo to afford N-[4-[[3-[1-(3-methyl-2-nitro-imidazol-4-yl)ethoxy]-7-morpholino-1,6-naphthyridin-5-yl]oxy]cyclohexyl]-5-(2-morpholinoethoxy)pyrimidin-2-amine (19.3 mg, 50% yield) as a yellow solid.

1H NMR (400 MHz, DMSO) δ: 8.69 (d, J=3.0 Hz, 1H), 8.12 (s, 2H), 7.76 (dd, J=3.0, 0.8 Hz, 1H), 7.37 (d, J=0.5 Hz, 1H), 6.69 (d, J=7.9 Hz, 1H), 6.51 (d, J=0.8 Hz, 1H), 5.97 (q, J=6.4 Hz, 1H), 5.40-5.36 (m, 1H), 4.05 (t, J=5.7 Hz, 2H), 3.99 (s, 3H), 3.80 (s, 1H), 3.76-3.69 (m, 4H), 3.60-3.53 (m, 4H), 3.46-3.39 (m, 4H), 2.63 (t, J=5.7 Hz, 2H), 2.47-2.41 (m, 4H), 2.12-1.97 (m, 2H), 1.83-1.67 (m, 9H)

LC-MS (long, acidic method) rt 1.78 min, 705.3 [M+H]+

### N-[1-(1-methyl-5-nitro-pyrazol-4-yl)ethyl]-N-[7-morpholino-5-[4-(pyrazolo[1,5-a]pyrimidin-5-ylamino)cyclohexoxy]-1,6-naphthyridin-3-yl]methanesulfonamide (LFA2050)

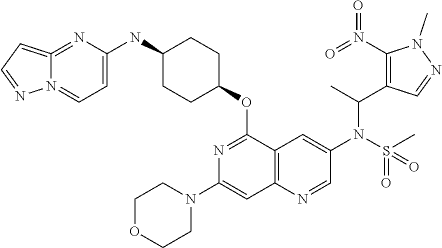

N-[7-morpholino-5-[4-(pyrazolo[1,5-a]pyrimidin-5-ylamino)cyclohexoxy]-1,6-naphthyridin-3-yl]methanesulfonamide (50 mg, 0.09 mmol), 4-(1-chloroethyl)-1-methyl-5-nitro-pyrazole (35.2 mg, 0.1900 mmol) and potassium carbonate (38.49 mg, 0.2800 mmol) were combined in DMF (1 mL) and heated to 100 C for 24 hours. The reaction mixture was allowed to cool to room temperature. The reaction was filtered through a syringe filter, washing the filter with DMSO. The reaction was purified by basic prep HPLC. The appropriate fractions were combined and the solvent removed in the genevac to afford N-[1-(1-methyl-5-nitro-pyrazol-4-yl)ethyl]-N-[7-morpholino-5-[4-(pyrazolo[1,5-a]pyrimidin-5-ylamino)cyclohexoxy]-1,6-naphthyridin-3-yl]methanesulfonamide (29 mg, 45% yield).

1H NMR (400 MHz, DMSO) δ: 8.43 (dd, J=7.6, 0.9 Hz, 1H), 8.32 (d, J=2.5 Hz, 1H), 7.86 (d, J=2.5 Hz, 1H), 7.75 (d, J=2.1 Hz, 1H), 7.38 (d, J=7.2 Hz, 1H), 7.21 (s, 1H), 6.54 (d, J=0.9 Hz, 1H), 6.30 (d, J=7.7 Hz, 1H), 5.96 (dd, J=2.1, 0.9 Hz, 1H), 5.92 (q, J=7.0 Hz, 1H), 5.38-5.34 (m, 1H), 4.11 (s, 3H), 4.05-4.00 (m, 1H), 3.74 (dd, J=5.8, 4.0 Hz, 4H), 3.54 (t, J=4.9 Hz, 4H), 3.11 (s, 3H), 2.06 (s, 2H), 1.94-1.81 (m, 4H), 1.77-1.64 (m, 2H), 1.56 (d, J=7.0 Hz, 3H).

LC-MS (acidic long method) RT=2.308 min, m/z 692.00 [M+H]+

### N-[1-(3-cyclopropyl-2-nitro-imidazol-4-yl)ethyl]-N-[7-morpholino-5-[4-(pyrazolo[1,5-a]pyrimidin-5-ylamino)cyclohexoxy]-1,6-naphthyridin-3-yl]methanesulfonamide (LFA2051)

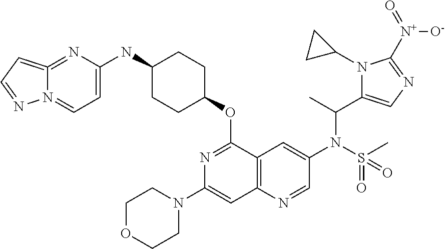

A mixture of N-[7-morpholino-5-[4-(pyrazolo[1,5-a]pyrimidin-5-ylamino)cyclohexoxy]-1,6-naphthyridin-3-yl]methanesulfonamide (39 mg, 0.07 mmol), 5-(1-chloroethyl)-1-cyclopropyl-2-nitro-imidazole (18.74 mg, 0.09 mmol) and Potassium carbonate (25.02 mg, 0.1800 mmol) in DMF (1 mL) was stirred at room temperature for 20 h, then at 50 C for 5 h. The reaction mixture was diluted with EtOAc (20 mL) and washed with brine (3×20 mL). The combined organic phases were dried (Na₂SO₄), filtered and concentrated in vacuo to give the crude product as an orange gum/glass (41 mg). The crude product was purified by flash column chromatography (10 g silica column, eluting with 0-5% MeOH in DCM) to give a yellow gum/glass (32 mg) (product with very minor impurities). The yellow gum/glass was further purified by basic prep. HPLC (method: basic prep.; 5-95% 0.005 M NH₄OH/MeCN in 0.005 M NH₄OH/H2O) to give the product, N-[1-(3-cyclopropyl-2-nitro-imidazol-4-yl)ethyl]-N-[7-morpholino-5-[4-(pyrazolo[1,5-a]pyrimidin-5-ylamino)cyclohexoxy]-1,6-naphthyridin-3-yl]methanesulfonamide (29 mg, 53% yield), as a yellow solid.

1H NMR (400 MHz, DMSO-d6) δ: 8.47-8.38 (m, 2H), 7.75 (d, J=2.1 Hz, 1H), 7.73 (d, J=2.5 Hz, 1H), 7.33 (d, J=7.5 Hz, 1H), 6.72 (d, J=0.6 Hz, 1H), 6.53 (d, J=0.8 Hz, 1H), 6.31 (d, J=7.6 Hz, 1H), 5.97 (dd, J=2.1, 0.9 Hz, 1H), 5.90 (q, J=6.9 Hz, 1H), 5.35-5.15 (m, 1H), 4.13-3.96 (m, 1H), 3.81-3.65 (m, 5H), 3.59-3.49 (m, 4H), 3.24 (s, 3H), 2.13-1.94 (m, 2H), 1.93-1.76 (m, 4H), 1.74-1.58 (m, 2H), 1.63 (d, J=7.0 Hz, 3H), 1.36 (tt, J=7.3, 3.8 Hz, 1H), 1.22 (ddd, J=9.2, 5.4, 3.3 Hz, 2H), 1.05-0.93 (m, 1H).

LCMS (long, acidic): 2.33 min, 718.2 [M+H]+

### N-[5-[4-[(5-cyanopyrimidin-2-yl)amino]cyclohexoxy]-7-morpholino-1,6-naphthyridin-3-yl]-N-[1-(1-methyl-5-nitro-pyrazol-4-yl)ethyl]methanesulfonamide (LFA2052)

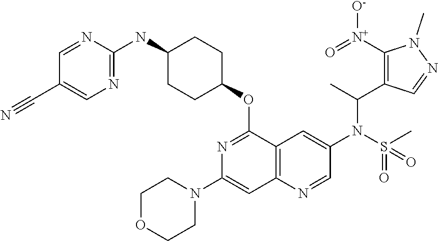 [SMILES: CC(c1cnn(C)c1[N+](=O)[O-])N(c1cnc2cc(N3CCOCC3)nc(O[C@H]3CC[C@@H](Nc4ncc(C#N)cn4)CC3)c2c1)S(C)(=O)=O]

N-[5-[4-[(5-cyanopyrimidin-2-yl)amino]cyclohexoxy]-7-morpholino-1,6-naphthyridin-3-yl]methanesulfonamide (50 mg, 0.10 mmol), 4-(1-chloroethyl)-1-methyl-5-nitro-pyrazole (19.88 mg, 0.10 mmol) LFA009 and potassium carbonate (39.5 mg, 0.2900 mmol) were combined in DMF (1 mL) at room temperature for 18 hours. The reaction mixture was heated to 100° C. for 24 hours. Further 4-(1-chloroethyl)-1-methyl-5-nitro-pyrazole (9.04 mg, 0.0500 mmol) was added and the reaction heated for a further 4 hours. The reaction mixture was purified by basic prep HPLC. The fractions were combined and the solvent removed at reduced pressure to afford a residue, which was purified by acidic prep HPLC. The fractions were combined, diluted with methanol and loaded onto a 500 mg SCX. The column was washed with methanol and eluted with 2M ammonia in methanol. The ammonia fraction was concentrated at reduced pressure to afford N-[5-[4-[(5-cyanopyrimidin-2-yl)amino]cyclohexoxy]-7-morpholino-1,6-naphthyridin-3-yl]-N-[1-(1-methyl-5-nitro-pyrazol-4-yl)ethyl]methanesulfonamide (5.4 mg, 8% yield).

1H NMR (400 MHz, DMSO) δ: 8.73 (d, J=3.1 Hz, 1H), 8.66 (d, J=3.0 Hz, 1H), 8.38-8.28 (m, 2H), 7.85 (s, 1H), 7.24 (s, 1H), 6.54 (s, 1H), 5.93 (q, J=7.0 Hz, 1H), 5.39 (s, 1H), 4.11 (s, 3H), 3.97 (s, 1H), 3.80-3.69 (m, 4H), 3.57-3.50 (m, 4H), 3.11 (s, 3H), 2.09 (s, 2H), 1.85-1.69 (m, 6H), 1.54 (d, J=6.9 Hz, 3H).

LC-MS (acidic long method) RT=2.517 min, m/z 678.2 [M+H]+

### N-[1-(3-methyl-2-nitro-imidazol-4-yl)ethyl]-N-[7-morpholino-5-[4-[[5-(morpholinomethyl)pyrimidin-2-yl]amino]cyclohexoxy]-1,6-naphthyridin-3-yl]methanesulfonamide (LFA2053)

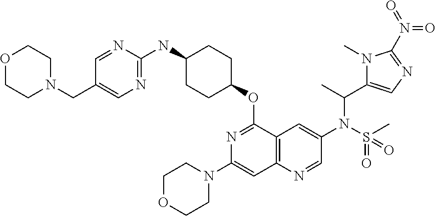 [SMILES: CC(c1cnc([N+](=O)[O-])n1C)N(c1cnc2cc(N3CCOCC3)nc(O[C@H]3CC[C@@H](Nc4ncc(CN5CCOCC5)cn4)CC3)c2c1)S(C)(=O)=O]

N-[7-morpholino-5-[4-[[5-(morpholinomethyl)pyrimidin-2-yl]amino]cyclohexoxy]-1,6-naphthyridin-3-yl]methanesulfonamide (25.mg, 0.0400 mmol), 5-(1-chloroethyl)-1-methyl-2-nitro-imidazole (8.71 mg, 0.0500 mmol) LFA009 and Potassium carbonate (17.31 mg, 0.1300 mmol) were transferred to a 10 mL RB flask, DMF (0.5000 mL) was added and the suspension stirred at room temperature for 20 h. The mixture was filtered through a syringe filter and washed through with DMSO (˜0.5 mL). The combined filtrate and washings was purified by basic prep HPLC (2 injections) and relevant fractions were combined and concentrated in vacuo to afford N-[1-(3-methyl-2-nitro-imidazol-4-yl)ethyl]-N-[7-morpholino-5-[4-[[5-(morpholinomethyl)pyrimidin-2-yl]amino]cyclohexoxy]-1,6-naphthyridin-3-yl]methanesulfonamide (15.4 mg, 49% yield) as a yellow solid.

1H NMR (400 MHz, DMSO) δ: 8.35 (d, J=2.5 Hz, 1H), 8.17 (s, 2H), 7.75-7.70 (m, 1H), 7.07 (d, J=7.9 Hz, 1H), 6.80 (d, J=0.6 Hz, 1H), 6.50 (d, J=0.8 Hz, 1H), 5.77-5.68 (m, 1H), 5.34-5.29 (m, 1H), 4.17 (s, 3H), 3.92-3.81 (m, 1H), 3.76-3.68 (m, 4H), 3.58-3.48 (m, 8H), 3.30 (s, 3H), 3.27 (s, 2H), 2.38-2.27 (m, 4H), 2.16-2.05 (m, 1H), 2.03-1.93 (m, 1H), 1.84-1.57 (m, 9H).

LC-MS (long, acidic method) rt 1.71 min 752.2 [M+H]+

### N-[1-(3-methyl-2-nitro-imidazol-4-yl)ethyl]-N-[7-morpholino-5-[4-[[5-(2-morpholinoethoxy)pyrimidin-2-yl]amino]cyclohexoxy]-1,6-naphthyridin-3-yl]methanesulfonamide (LFA2054)

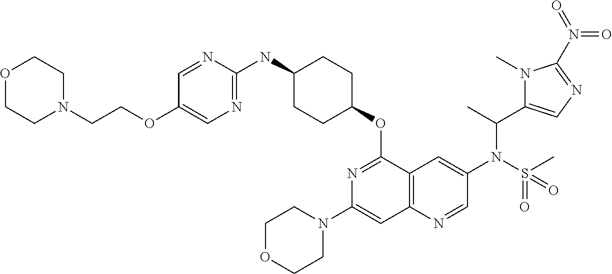 [SMILES: CC(c1cnc([N+](=O)[O-])n1C)N(c1cnc2cc(N3CCOCC3)nc(O[C@H]3CC[C@@H](Nc4ncc(OCCN5CCOCC5)cn4)CC3)c2c1)S(C)(=O)=O]

N-[7-morpholino-5-[4-[[5-(2-morpholinoethoxy)pyrimidin-2-yl]amino]cyclohexoxy]-1,6-naphthyridin-3-yl]methanesulfonamide (60.mg, 0.1000 mmol), 5-(1-chloroethyl)-1-methyl-2-nitro-imidazole (19.9 mg, 0.1000 mmol) LFA009 and potassium carbonate (39.57 mg, 0.2900 mmol) were combined in DMF (1 mL) at room temperature for 72 hours. The reaction mixture was filtered and purified by basic prep HPLC. The fractions were loaded onto a 1 g SCX. The column was washed with methanol and eluted with 2M ammonia in methanol. The ammonia fraction was concentrated at reduced pressure to afford N-[1-(3-methyl-2-nitro-imidazol-4-yl)ethyl]-N-[7-morpholino-5-[4-[[5-(2-morpholinoethoxy)pyrimidin-2-yl]amino]cyclohexoxy]-1,6-naphthyridin-3-yl]methanesulfonamide (45 mg, 60% yield).

1H NMR (400 MHz, CDCl3) δ: 8.42 (d, J=2.6 Hz, 1H), 8.09 (s, 2H), 7.66 (dd, J=2.6, 0.8 Hz, 1H), 6.65 (d, J=0.7 Hz, 1H), 6.45 (d, J=0.9 Hz, 1H), 5.83 (q, J=7.0 Hz, 1H), 5.44 (d, J=8.2 Hz, 1H), 5.32 (s, 1H), 4.29 (s, 3H), 4.09 (s, 2H), 3.95-3.88 (m, 1H), 3.84 (t, J=4.9 Hz, 4H), 3.79-3.70 (m, 3H), 3.64-3.51 (m, 4H), 3.07 (s, 3H), 2.81-2.77 (m, 1H), 2.61-2.54 (m, 2H), 2.16-2.05 (m, 2H), 1.97-1.90 (m, 2H), 1.88-1.76 (m, 2H), 1.73 (d, J=7.1 Hz, 3H), 1.66-1.55 (m, 6H).

LC-MS (acidic long method) RT=1.721 min, m/z 782.2 [M+H]+

### N-[1-(3-cyclopropyl-2-nitro-imidazol-4-yl)ethyl]-N-[7-morpholino-5-[4-(pyrimidin-2-ylamino)cyclohexoxy]-1,6-naphthyridin-3-yl]methanesulfonamide (LFA2055)

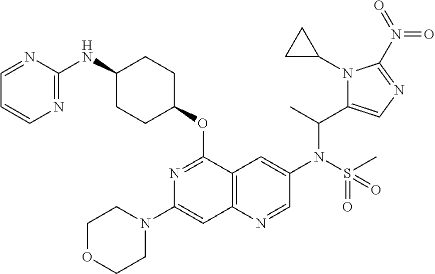 [SMILES: CC(c1cnc([N+](=O)[O-])n1C1CC1)N(c1cnc2cc(N3CCOCC3)nc(O[C@H]3CC[C@@H](Nc4ncccn4)CC3)c2c1)S(C)(=O)=O]

A mixture of N-[7-morpholino-5-[4-(pyrimidin-2-ylamino)cyclohexoxy]-1,6-naphthyridin-3-yl]methanesulfonamide (24.mg, 0.0500 mmol), 5-(1-chloroethyl)-1-cyclopropyl-2-nitro-imidazole (12.43 mg, 0.0600 mmol) and Potassium carbonate (16.6 mg, 0.1200 mmol) in DMF (1 mL) was stirred at room temperature for 20 h, then at 50 C for 4 h. The reaction mixture was diluted with EtOAc (20 mL) and washed with water (20 mL) and brine (2×20 mL). The combined organic phases were dried (Na₂SO₄), filtered and concentrated in vacuo to give the crude product as an orange gum/glass (37 mg). The crude product was purified by flash column chromatography (10 g silica column, eluting with 0-6% MeOH in DCM) to give impure product as yellow gum/glass (23 mg). The product was further purified by basic prep. HPLC (method: basic prep.; 5-95% 0.005 M NH₄OH/MeCN in 0.005 M NH₄OH/H2O) to give the product, N-[1-(3-cyclopropyl-2-nitro-imidazol-4-yl)ethyl]-N-[7-morpholino-5-[4-(pyrimidin-2-ylamino)cyclohexoxy]-1,6-naphthyridin-3-yl]methanesulfonamide (17.5 mg, 51% yield), as a yellow solid.

1H NMR (400 MHz, DMSO-d6) δ: 8.41 (d, J=2.5 Hz, 1H), 8.27 (d, J=4.7 Hz, 2H), 7.75 (s, 1H), 7.00 (d, J=7.8 Hz, 1H), 6.71 (d, J=0.6 Hz, 1H), 6.55 (t, J=4.8 Hz, 1H), 6.52 (d, J=0.8 Hz, 1H), 5.90 (q, J=7.0 Hz, 1H), 5.40-5.22 (m, 1H), 3.93-3.82 (m, 1H), 3.78-3.66 (m, 5H), 3.58-3.47 (m, 4H), 3.24 (s, 3H), 2.20-2.05 (m, 1H), 2.03-1.94 (m, 1H), 1.85-1.69 (m, 4H), 1.66 (d, 1.71-1.59 (m, 2H), J=7.0 Hz, 3H), 1.40-1.30 (m, 1H), 1.29-1.17 (m, 2H), 1.04-0.93 (m, 1H).

LCMS (short, acidic): 1.65 min, 679.2 [M+H]+

### N-[1-(1-methyl-5-nitro-pyrazol-4-yl)ethyl]-N-[7-morpholino-5-[4-(pyrimidin-2-ylamino)cyclohexoxy]-1,6-naphthyridin-3-yl]methanesulfonamide (LFA2056)

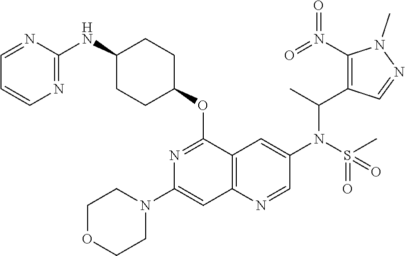 [SMILES: CC(c1cnn(C)c1[N+](=O)[O-])N(c1cnc2cc(N3CCOCC3)nc(O[C@H]3CC[C@@H](Nc4ncccn4)CC3)c2c1)S(C)(=O)=O]

N-[7-morpholino-5-[4-(pyrimidin-2-ylamino)cyclohexoxy]-1,6-naphthyridin-3-yl]methanesulfonamide (25.mg, 0.0500 mmol), 4-(1-chloroethyl)-1-methyl-5-nitro-pyrazole (10.44 mg, 0.0600 mmol) and Potassium carbonate (20.75 mg, 0.1500 mmol) were transferred to a 5 mL RB flask and DMF (0.5000 mL) was added. The mixture was stirred under an argon atmosphere for 18 h and then at 60-C for 3 h and 80-C for 1 h and 100-C for 4 h. The mixture was cooled to room temperature, filtered through a syringe filter, washed through with DMSO (˜300 uL) and purified by acidic prep HPLC (2×400 uL injections). Relevant fractions were combined, loaded onto a SCX column (1 g) in MeOH, washed with MeOH and the product eluted off with 2 M ammonia in methanol. Concentration of relevant fractions under reduced pressure afforded a dark orange solid yield 15 mg. Further purified by loading onto a 5 g silica column in DCM and eluting with MeOH (0-5%) in DCM. Relevant fractions were combined and concentrated to afford N-[1-(1-methyl-5-nitro-pyrazol-4-yl)ethyl]-N-[7-morpholino-5-[4-(pyrimidin-2-ylamino)cyclohexoxy]-1,6-naphthyridin-3-yl]methanesulfonamide (14.2 mg, 43% yield) as an orange solid.

1H NMR (400 MHz, DMSO) δ: 8.39 (d, J=2.6 Hz, 1H), 8.27 (d, J=4.8 Hz, 2H), 7.84 (d, J=2.5 Hz, 1H), 7.27 (s, 1H), 7.05 (d, J=7.8 Hz, 1H), 6.58-6.51 (m, 2H), 5.93 (q, J=7.0 Hz, 1H), 5.42-5.35 (m, 1H), 4.13 (s, 3H), 3.96-3.84 (m, 1H), 3.78-3.69 (m, 4H), 3.58-3.49 (m, 4H), 3.11 (s, 3H), 2.14-2.03 (m, 2H), 1.86-1.60 (m, 6H), 1.54 (d, J=7.0 Hz, 3H).

LCMS (long, acidic): rt 2.26 min, 653.2 [M+H]+

### N-[(1-methyl-5-nitro-pyrazol-4-yl)methyl]-N-[7-morpholino-5-[4-(pyrimidin-2-ylamino)cyclohexoxy]-1,6-naphthyridin-3-yl]methanesulfonamide (LFA2057)

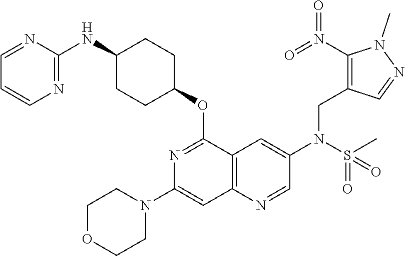 [SMILES: Cn1ncc(CN(c2cnc3cc(N4CCOCC4)nc(O[C@H]4CC[C@@H](Nc5ncccn5)CC4)c3c2)S(C)(=O)=O)c1[N+](=O)[O-]]

N-[7-morpholino-5-[4-(pyrimidin-2-ylamino)cyclohexoxy]-1,6-naphthyridin-3-yl]methanesulfonamide (25.mg, 0.0500 mmol), (1-methyl-5-nitro-pyrazol-4-yl)methyl methane sulfonate (12.95 mg, 0.0600 mmol) and Potassium carbonate (20.75 mg, 0.1500 mmol) were transferred to a 5 mL RB flask and DMF (0.5000 mL) was added. The mixture was stirred under an argon atmosphere for 24 h. The mixture was filtered through a syringe filter, washed through with DMSO (˜300 uL) and purified by acidic prep HPLC (2×400 uL injections). Relevant fractions were combined, loaded onto a SCX column (1 g) in MeOH, washed with MeOH and the product eluted off with 2 M ammonia in methanol. Concentration under reduced pressure and further drying in a vacuum oven at 40 C for 4 h afforded N-[(1-methyl-5-nitro-pyrazol-4-yl) methyl]-N-[7-morpholino-5-[4-(pyrimidin-2-ylamino)cyclohexoxy]-1,6-naphthyridin-3-yl]methanesulfonamide (21 mg, 66% yield) as a pale orange solid.

1H NMR (400 MHz, DMSO) δ: 8.79 (d, J=2.6 Hz, 1H), 8.27 (d, J=4.8 Hz, 2H), 8.21 (dd, J=2.6, 0.8 Hz, 1H), 7.72 (s, 1H), 6.99 (d, J=7.8 Hz, 1H), 6.55 (t, J=4.7 Hz, 1H), 6.53 (d, J=0.9 Hz, 1H), 5.40-5.36 (m, 1H), 5.11 (s, 2H), 4.05 (s, 3H), 3.96-3.85 (m, 1H), 3.77-3.68 (m, 4H), 3.54-3.47 (m, 4H), 3.22 (s, 3H), 2.13-2.01 (m, 2H), 1.83-1.71 (m, 6H).

LCMS (long, acidic): rt 2.23 min, 639.2 [M+H]+

### N-[(3-cyclopropyl-2-nitro-imidazol-4-yl)methyl]-N-[7-morpholino-5-[4-(pyrimidin-2-ylamino)cyclohexoxy]-1,6-naphthyridin-3-yl]methanesulfonamide (LFA2058)

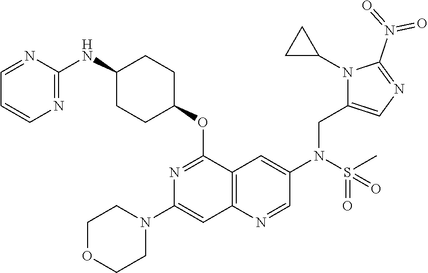 [SMILES: CS(=O)(=O)N(Cc1cnc([N+](=O)[O-])n1C1CC1)c1cnc2cc(N3CCOCC3)nc(O[C@H]3CC[C@@H](Nc4ncccn4)CC3)c2c1]

A mixture of N-[7-morpholino-5-[4-(pyrimidin-2-ylamino)cyclohexoxy]-1,6-naphthyridin-3-yl]methanesulfonamide (25.mg, 0.0500 mmol), 5-(chloromethyl)-1-cyclopropyl-2-nitro-imidazole (12.11 mg, 0.0600 mmol) and Potassium carbonate (17.29 mg, 0.1300 mmol) in DMF (1 mL) was stirred at room temperature for 20 h. The reaction mixture was diluted with EtOAc (20 mL) and washed with water (20 mL) and brine (2×20 mL). The combined organic phases were dried (Na₂SO₄), filtered and concentrated in vacuo to give the crude product as an orange gum/glass (47 mg). The crude product was purified by flash column chromatography (10 g silica column, eluting with 0-6% MeOH in DCM) to give the product as a yellow/orange solid (15 mg, 45%). The solid was triturated with MeOH (2 mL), filtered, rinsed with MeOH (2×1 mL) and dried in a vacuum oven at 40 C for 16 h to give the product, N-[(3-cyclopropyl-2-nitro-imidazol-4-yl)methyl]-N-[7-morpholino-5-[4-(pyrimidin-2-ylamino)cyclohexoxy]-1,6-naphthyridin-3-yl]methanesulfonamide (8 mg, 24% yield), as a bright yellow solid.

1H NMR (400 MHz, DMSO-d6) δ: 8.83 (d, J=2.6 Hz, 1H), 8.27 (d, J=4.8 Hz, 2H), 8.22 (dd, J=2.6, 0.8 Hz, 1H), 7.03 (s, 1H), 7.02 (d, J=8.5 Hz, 1H), 6.55 (t, J=4.7 Hz, 1H), 6.52 (d, J=0.9 Hz, 1H), 5.42-5.35 (m, 1H), 5.20 (s, 2H), 3.95-3.82 (m, 1H), 3.76-3.67 (m, 4H), 3.54-3.47 (m, 4H), 3.47-3.38 (m, 1H), 3.24 (s, 3H), 2.15-2.00 (m, 2H), 1.87-1.69 (m, 6H), 1.18-1.08 (m, 2H), 1.04-0.94 (m, 2H).

LCMS (short, acidic): 1.62 min, 665.2 [M+H]+

### N-[(3-methyl-2-nitro-imidazol-4-yl)methyl]-N-[5-[4-[[5-[2-(4-methylpiperazin-1-yl)ethoxy]pyrimidin-2-yl]amino]cyclohexoxy]-7-morpholino-1,6-naphthyridin-3-yl]methanesulfonamide (LFA2059)

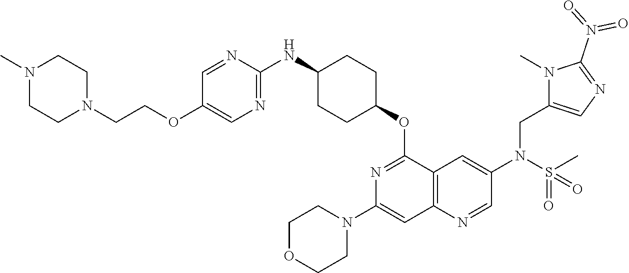 [SMILES: CN1CCN(CCOc2cnc(N[C@H]3CC[C@@H](Oc4nc(N5CCOCC5)cc5ncc(N(Cc6cnc([N+](=O)[O-])n6C)S(C)(=O)=O)cc45)CC3)nc2)CC1]

N-[5-[4-[[5-[2-(4-methylpiperazin-1-yl)ethoxy]pyrimidin-2-yl]amino]cyclohexoxy]-7-morpholino-1,6-naphthyridin-3-yl]methanesulfonamide (47.mg, 0.0700 mmol), 5-(bromomethyl)-1-methyl-2-nitro-imidazole (17.72 mg, 0.0800 mmol) LFA007 and Potassium carbonate (30.36 mg, 0.2200 mmol) were combined in DMF (1 mL) at room temperature and stirred under an argon atmosphere for 18 hours. The reaction was concentrated at reduced pressure. The resulting residue was taken up in DCM and washed with water. The organic was dried (Na₂SO₄) and the solvent removed at reduced pressure. The resulting residue was purified by flash chromatography (5 g SiO) eluting with a gradient 0-10% of 10% 2M ammonia in methanol in DCM. The appropriate fractions were combined and the solvent removed at reduced pressure to afford residue, which was further purified by prep acidic HPLC. The appropriate fractions were loaded onto a 500 mg SCX column. The column was washed with methanol and eluted with 2M ammonia in methanol. The ammonia fraction was concentrated at reduced pressure to afford N-[(3-methyl-2-nitro-imidazol-4-yl)methyl]-N-[5-[4-[[5-[2-(4-methylpiperazin-1-yl)ethoxy]pyrimidin-2-yl]amino]cyclohexoxy]-7-morpholino-1,6-naphthyridin-3-yl]methanesulfonamide (7.4 mg, 13% yield).

1H NMR (400 MHz, DMSO) δ: 8.70 (d, J=2.6 Hz, 1H), 8.18 (dd, J=2.6, 0.8 Hz, 1H), 8.12 (s, 2H), 6.98 (s, 1H), 6.68 (d, J=7.6 Hz, 1H), 6.51 (s, 1H), 5.41-5.30 (m, 2H), 5.15 (s, 2H), 4.03 (t, J=5.8 Hz, 2H), 3.95 (s, 3H), 3.80 (s, 1H), 3.75-3.69 (m, 4H), 3.54-3.47 (m, 4H), 3.25 (s, 3H), 2.62 (t, J=5.8 Hz, 2H), 2.44 (s, 3H), 2.30 (s, 3H), 2.14 (s, 3H), 2.11-2.00 (m, 3H), 1.83-1.69 (m, 6H).

LCMS (long, acidic): rt 1.636 min, 781.2 [M+H]+

### N-[1-(3-methyl-2-nitro-imidazol-4-yl)ethyl]-N-[5-[4-[[5-[2-(4-methylpiperazin-1-yl)ethoxy]pyrimidin-2-yl]amino]cyclohexoxy]-7-morpholino-1,6-naphthyridin-3-yl]methanesulfonamide (LFA2060)

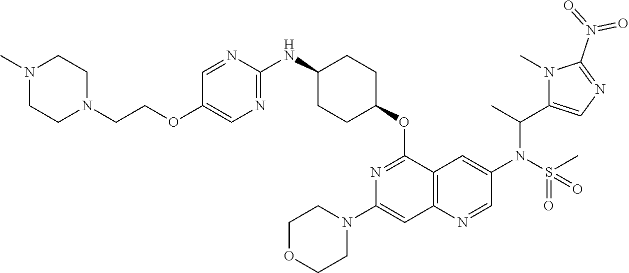 [SMILES: CC(c1cnc([N+](=O)[O-])n1C)N(c1cnc2cc(N3CCOCC3)nc(O[C@H]3CC[C@@H](Nc4ncc(OCCN5CCN(C)CC5)cn4)CC3)c2c1)S(C)(=O)=O]

A mixture of N-[5-[4-[[5-[2-(4-methylpiperazin-1-yl)ethoxy]pyrimidin-2-yl]amino]cyclohexoxy]-7-morpholino-1,6-naphthyridin-3-yl]methanesulfonamide (235.mg, 0.3700 mmol), 5-(1-chloroethyl)-1-methyl-2-nitro-imidazole (76.37 mg, 0.4000 mmol) LFA009 and Potassium carbonate (126.52 mg, 0.9200 mmol) in DMF (3.7 mL) was stirred at RT over the weekend. The mixture was filtered through a syringe filter and washed through with DMSO (1 mL). The combined filtrate and washings were loaded onto a 60 g Biotage SNAP KP-C18-HS cartridge and eluted with 0.1% TFA in MeCN (5-100%) in 0.1% TFA in water (gradient over 20 column volumes, flow rate 40 mL/min). Two fractions were collected. First product fractions (containing a more polar impurity MH+771 were combined and free based on a 10 g SCX cartridge to afford 63.5 mg yellow solid. Further purified by basic prep HPLC (4×300 uL) injections in DMSO) to give a yellow solid yield 35 mg.

Latter fractions from RP Biotage column were combined and free based on a 10 g SCX to give 81.5 mg yellow solid. Further purified by basic prep HPLC (4×250 uL injections in DMSO). Relevant fractions were combined and concentrated to afford a yellow solid yield 51.3 mg

Both solids were combined and dried further in a vacuum oven at 40 C for 20 h to afford N-[1-(3-methyl-2-nitro-imidazol-4-yl)ethyl]-N-[5-[4-[[5-[2-(4-methylpiperazin-1-yl)ethoxy]pyrimidin-2-yl]amino]cyclohexoxy]-7-morpholino-1,6-naphthyridin-3-yl]methanesulfonamide (78.2 mg, 48% yield) as a yellow solid.

1H NMR (400 MHz, DMSO) δ: 8.35 (d, J=2.5 Hz, 1H), 8.11 (s, 2H), 7.72 (s, 1H), 6.80 (d, J=0.6 Hz, 1H), 6.71 (d, J=7.8 Hz, 1H), 6.50 (d, J=0.8 Hz, 1H), 5.72 (q, J=6.9 Hz, 1H), 5.33-5.28 (m, 1H), 4.17 (s, 3H), 4.03 (t, J=5.8 Hz, 2H), 3.81-3.75 (m, 1H), 3.75-3.69 (m, 4H), 3.55-3.48 (m, 4H), 3.30 (s, 3H), 2.62 (t, J=5.8 Hz, 2H), 2.49-2.17 (m, 8H), 2.13 (s, 3H), 2.11-2.02 (m, 1H), 2.01-1.93 (m, 1H), 1.82-1.55 (m, 9H).

LC-MS (long, acidic method) rt 1.65 min, 795.2 [M+H]+

### N-[5-[4-[[5-[2-(dimethylamino)ethoxy]pyrimidin-2-yl]amino]cyclohexoxy]-7-morpholino-1,6-naphthyridin-3-yl]-N-[1-(3-methyl-2-nitro-imidazol-4-yl)ethyl]methanesulfonamide (LFA2061)

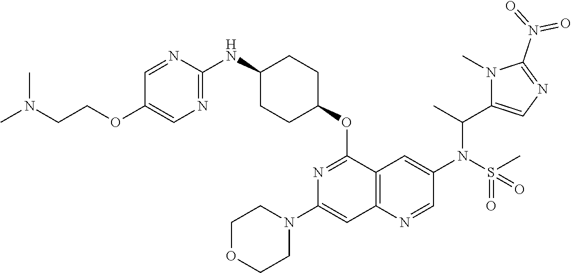 [SMILES: CC(c1cnc([N+](=O)[O-])n1C)N(c1cnc2cc(N3CCOCC3)nc(O[C@H]3CC[C@@H](Nc4ncc(OCCN(C)C)cn4)CC3)c2c1)S(C)(=O)=O]

N-[5-[4-[[5-[2-(dimethylamino)ethoxy]pyrimidin-2-yl]amino]cyclohexoxy]-7-morpholino-1,6-naphthyridin-3-yl]methanesulfonamide (35.mg, 0.0600 mmol) was transferred to a 5 mL microwave vial and Potassium carbonate (24.73 mg, 0.1800 mmol) added followed by a solution of 5-(1-chloroethyl)-1-methyl-2-nitro-imidazole (12.44 mg, 0.0700 mmol) LFA009 in DMF (0.5000 mL). The mixture was stirred under an argon atmosphere at room temperature for 16 h and then filtered through a syringe filter and washed through with DMSO (200 uL). Combined filtrate and washings purified by basic prep HPLC (2×350 uL) and relevant fractions were concentrated to dryness under reduced pressure to afford N-[5-[4-[[5-[2-(dimethylamino)ethoxy]pyrimidin-2-yl]amino]cyclohexoxy]-7-morpholino-1,6-naphthyridin-3-yl]-N-[1-(3-methyl-2-nitro-imidazol-4-yl)ethyl]methanesulfonamide (14.7 mg, 33% yield) as an orange solid.

1H NMR (400 MHz, DMSO) δ: 8.35 (d, J=2.6 Hz, 1H), 8.12 (s, 2H), 7.71 (d, 1H), 6.80 (d, J=0.6 Hz, 1H), 6.70 (d, J=7.8 Hz, 1H), 6.50 (s, 1H), 5.72 (q, J=6.9 Hz, 1H), 5.34-5.27 (m, 1H), 4.17 (s, 3H), 4.01 (t, J=5.8 Hz, 2H), 3.84-3.75 (m, 1H), 3.75-3.69 (m, 4H), 3.55-3.48 (m, 4H), 3.30 (s, 3H), 2.56 (t, J=5.8 Hz, 2H), 2.19 (s, 6H), 2.14-2.04 (m, 1H), 2.02-1.92 (m, 1H), 1.84-1.55 (m, 9H).

LCMS (long, acidic): 1.69 min, 740.3 [M+H]+

### N-[(1-methyl-5-nitro-pyrazol-4-yl)methyl]-N-[5-[4-[[5-(1-methylpyrrolidin-3-yl)oxypyrimidin-2-yl]amino]cyclohexoxy]-7-morpholino-1,6-naphthyridin-3-yl]methanesulfonamide (LFA2062)

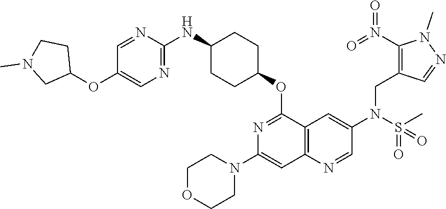 [SMILES: CN1CCC(Oc2cnc(N[C@H]3CC[C@@H](Oc4nc(N5CCOCC5)cc5ncc(N(Cc6cnn(C)c6[N+](=O)[O-])S(C)(=O)=O)cc45)CC3)nc2)C1]

N-[5-[4-[[5-(1-methylpyrrolidin-3-yl)oxypyrimidin-2-yl]amino]cyclohexoxy]-7-morpholino-1,6-naphthyridin-3-yl]methanesulfonamide (44.mg, 0.0700 mmol), 4-(chloromethyl)-1-methyl-5-nitro-pyrazole (14.19 mg, 0.0800 mmol) and potassium carbonate (30.47 mg, 0.2200 mmol) were combined in DMF (0.8000 mL) and stirred at room temperature for 17 h. Mixture filtered through a syringe filter and washed through with DMSO (200 uL) and purified by basic prep HPLC (3×300 uL). Relevant fractions were combined and concentrated to afford an orange gum yield 9.8 mg. Purified further by acidic HPLC (1×300 uL injection in DMSO) and relevant fractions free based by loading onto a 2 g SCX cartridge, washing with MeOH and eluting product off with 2M ammonia in MeOH. Relevant fractions were concentrated to dryness under reduced pressure to give N-[(1-methyl-5-nitro-pyrazol-4-yl)methyl]-N-[5-[4-[[5-(1-methylpyrrolidin-3-yl)oxypyrimidin-2-yl]amino]cyclohexoxy]-7-morpholino-1,6-naphthyridin-3-yl]methanesulfonamide (5 mg, 9%) an orange solid.

1H NMR (400 MHz, DMSO) δ: 8.79 (dd, J=2.6, 1.0 Hz, 1H), 8.20 (d, J=2.6 Hz, 1H), 8.07 (d, J=1.1 Hz, 2H), 7.72 (s, 1H), 6.62 (d, J=7.7 Hz, 1H), 6.53 (s, 1H), 5.42-5.32 (m, 1H), 5.11 (s, 2H), 4.81-4.72 (m, 1H), 4.05 (s, 3H), 3.87-3.76 (m, 1H), 3.76-3.69 (m, 4H), 3.54-3.47 (m, 4H), 3.22 (s, 3H), 2.73-2.55 (m, 3H), 2.36-2.30 (m, 1H), 2.27-2.14 (m, 4H), 2.13-1.99 (m, 2H), 1.85-1.67 (m, 7H).

LCMS (long, acidic): 1.76 min, 738.2 [M+H]+

### N-methyl-N-[4-[[3-[1-(3-methyl-2-nitro-imidazol-4-yl)ethoxy]-7-morpholino-1,6-naphthyridin-5-yl]oxy]cyclohexyl]pyrimidin-2-amine (LFA2063)

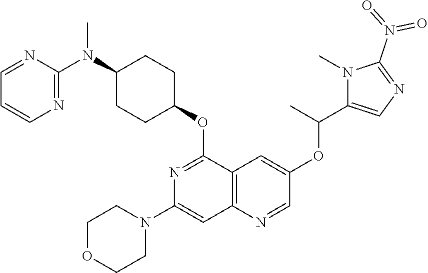 [SMILES: CC(Oc1cnc2cc(N3CCOCC3)nc(O[C@H]3CC[C@@H](N(C)c4ncccn4)CC3)c2c1)c1cnc([N+](=O)[O-])n1C]

A mixture of 5-[4-[methyl(pyrimidin-2-yl)amino]cyclohexoxy]-7-morpholino-1,6-naphthyridin-3-ol (50.mg, 0.1100 mmol), 5-(1-chloroethyl)-1-methyl-2-nitro-imidazole (26.06 mg, 0.1400 mmol) LFA009 and Potassium carbonate (39.58 mg, 0.2900 mmol) in DMF (1.2 mL) was stirred at RT for 17 h. The reaction mixture was diluted with water (10 mL) and the resultant precipitate filtered and washed with water (2×3 mL). The collected solid was dried by drawing air through for 10 min, then washed through with DCM (30 mL). The DCM solution was concentrated in vacuo to give the crude product as an orange gummy solid (59 mg). The gummy solid was purified by basic prep. HPLC (method: basic prep.; 5-95% 0.005 M NH₄OH/MeCN in 0.005 M NH₄OH/H2O) to give the product, N-methyl-N-[4-[[3-[1-(3-methyl-2-nitro-imidazol-4-yl)ethoxy]-7-morpholino-1,6-naphthyridin-5-yl]oxy]cyclohexyl]pyrimidin-2-amine (41 mg, 61% yield), as a yellow solid.

1H NMR (400 MHz, DMSO-d6) δ: 8.68 (d, J=3.0 Hz, 1H), 8.35 (d, J=4.7 Hz, 2H), 7.85 (dd, J=3.0, 0.8 Hz, 1H), 7.37 (s, 1H), 6.58 (t, J=4.7 Hz, 1H), 6.54 (d, J=0.8 Hz, 1H), 6.03 (q, J=6.4 Hz, 1H), 5.47-5.37 (m, 1H), 4.76 (tt, J=12.2, 3.8 Hz, 1H), 3.98 (s, 3H), 3.74 (dd, J=5.7, 4.1 Hz, 4H), 3.50-3.39 (m, 4H), 3.03 (s, 3H), 2.26-2.10 (m, 2H), 2.09-1.94 (m, 2H), 1.89-1.76 (m, 2H), 1.74 (d, J=6.5 Hz, 3H), 1.58-1.48 (m, 2H).

LCMS (long, acidic): 2.45 min, 590.2 [M+H]+

### N-[1-(1-methyl-5-nitro-pyrazol-4-yl)ethyl]-N-[5-[4-[[5-(1-methylpyrrolidin-3-yl)oxypyrimidin-2-yl]amino]cyclohexoxy]-7-morpholino-1,6-naphthyridin-3-yl]methanesulfonamide (LFA2064)

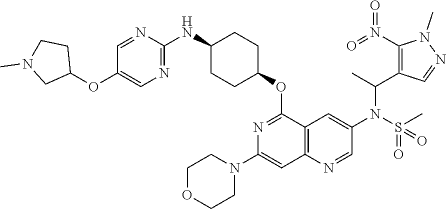 [SMILES: CC(c1cnn(C)c1[N+](=O)[O-])N(c1cnc2cc(N3CCOCC3)nc(O[C@H]3CC[C@@H](Nc4ncc(OC5CCN(C)C5)cn4)CC3)c2c1)S(C)(=O)=O]

A mixture of N-[5-[4-[[5-(1-methylpyrrolidin-3-yl)oxypyrimidin-2-yl]amino]cyclohexoxy]-7-morpholino-1,6-naphthyridin-3-yl]methanesulfonamide (50.mg, 0.0800 mmol), 4-(1-chloroethyl)-1-methyl-5-nitro-pyrazole (19.mg, 0.1000 mmol) and Potassium carbonate (28.86 mg, 0.2100 mmol) in DMF (1 mL) was heated to 100 C and stirred for 18 h. A further portion of 4-(1-chloroethyl)-1-methyl-5-nitro-pyrazole (10.mg, 0.0500 mmol) was added and the mixture was re-heated to 100 C and stirred for 5 h. After cooling to RT, the mixture was diluted with EtOAc (20 mL) and washed with water (20 mL). The aqueous phase was extracted with 10% MeOH/EtOAc (2×20 mL). The combined organic phases were washed with brine (2×40 mL), dried (Na₂SO₄), filtered and concentrated in vacuo to give the crude product as a brown/orange gum (61 mg). The gum was purified by flash column chromatography (10 g silica column, eluting with 0-7% 2 M NH₃/MeOH in DCM) then by basic prep. HPLC (method: basic prep.; 5-95% 0.005 M NH₄OH/MeCN in 0.005 M NH₄OH/H2O) to give the product, N-[1-(1-methyl-5-nitro-pyrazol-4-yl)ethyl]-N-[5-[4-[[5-(1-methylpyrrolidin-3-yl)oxypyrimidin-2-yl]amino]cyclohexoxy]-7-morpholino-1,6-naphthyridin-3-yl]methanesulfonamide (11 mg, 18% yield), as a yellow solid.

1H NMR (400 MHz, DMSO-d6) δ: 8.39 (d, J=2.6 Hz, 1H), 8.07 (s, 2H), 7.83 (d, J=2.5 Hz, 1H), 7.27 (s, 1H), 6.67 (d, J=7.8 Hz, 1H), 6.54 (d, J=0.9 Hz, 1H), 5.93 (q, J=7.0 Hz, 1H), 5.44-5.32 (m, 1H), 4.76 (ddd, J=10.5, 5.8, 2.9 Hz, 1H), 4.13 (s, 3H), 3.90-3.77 (m, 1H), 3.78-3.67 (m, 4H), 3.58-3.49 (m, 4H), 3.11 (s, 3H), 2.75-2.55 (m, 4H), 2.37-2.29 (m, 1H), 2.24 (s, 3H), 2.24-2.15 (m, 1H), 2.14-2.02 (m, 2H), 1.88-1.61 (m, 6H), 1.54 (d, J=7.1 Hz, 3H).

LCMS (long, acidic): 1.78 min, 752.2 [M+H]+

### N-[4-[[3-[(3-methyl-2-nitro-imidazol-4-yl)methoxy]-7-morpholino-1,6-naphthyridin-5-yl]oxy]cyclohexyl]-5-(4-methylpiperazin-1-yl)pyrimidin-2-amine (LFA2065)

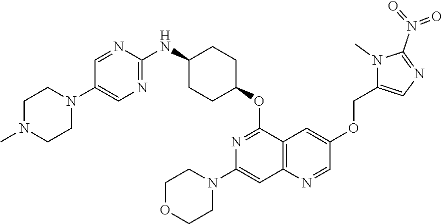 [SMILES: CN1CCN(c2cnc(N[C@H]3CC[C@@H](Oc4nc(N5CCOCC5)cc5ncc(OCc6cnc([N+](=O)[O-])n6C)cc45)CC3)nc2)CC1]

A mixture of 5-[4-[[5-(4-methylpiperazin-1-yl)pyrimidin-2-yl]amino]cyclohexoxy]-7-morpholino-1,6-naphthyridin-3-ol (50.mg, 0.1000 mmol), 5-(chloromethyl)-1-methyl-2-nitro-imidazole (18.55 mg, 0.1100 mmol) and Potassium carbonate (33.18 mg, 0.2400 mmol) in DMF (1 mL) was stirred at RT for 21 h. A further portion of 5-(chloromethyl)-1-methyl-2-nitro-imidazole (8.mg, 0.0500 mmol) was added and the mixture was stirred at RT for 5 h. The reaction mixture was diluted with EtOAc (20 mL) and washed with water (20 mL). The aqueous phase was extracted with 10% MeOH/EtOAc (2×20 mL). The combined organic phases were washed with brine (2×40 mL), dried (Na₂SO₄), filtered and concentrated in vacuo to give the crude product as a brown/orange gum (53 mg). The crude product was purified by flash column chromatography (10 g silica column, eluting with 0-5% 2 M NH₃/MeOH in DCM) to give impure product as a yellow/orange gum (40 mg). The yellow/orange gum was further purified by basic prep. HPLC (method: basic prep.; 5-95% 0.005 M NH₄OH/MeCN in 0.005 M NH₄OH/H2O) to give the product, N-[4-[[3-[(3-methyl-2-nitro-imidazol-4-yl)methoxy]-7-morpholino-1,6-naphthyridin-5-yl]oxy]cyclohexyl]-5-(4-methylpiperazin-1-yl)pyrimidin-2-amine (23 mg, 36% yield), as a yellow solid.

1H NMR (400 MHz, DMSO-d6) δ: 8.69 (d, J=3.0 Hz, 1H), 8.09 (s, 2H), 7.84 (dd, J=3.0, 0.8 Hz, 1H), 7.39 (s, 1H), 6.58-6.51 (m, 2H), 5.44 (s, 2H), 5.43-5.37 (m, 1H), 4.01 (s, 3H), 3.86-3.79 (m, 1H), 3.74 (dd, J=5.9, 3.8 Hz, 4H), 3.44 (dd, J=6.2, 3.6 Hz, 4H), 3.00-2.90 (m, 4H), 2.46-2.40 (m, 4H), 2.20 (s, 3H), 2.16-2.01 (m, 2H), 1.87-1.72 (m, 6H).

LCMS (long, acidic): 1.74 min, 660.3 [M+H]+

### N-[4-[[3-[1-(3-methyl-2-nitro-imidazol-4-yl)ethoxy]-7-morpholino-1,6-naphthyridin-5-yl]oxy]cyclohexyl]-5-(4-methylpiperazin-1-yl)pyrimidin-2-amine (LFA2066)

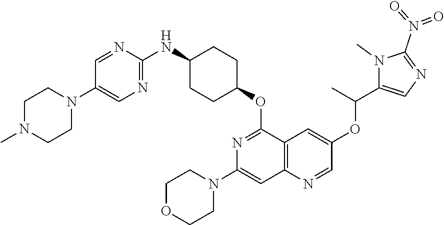 [SMILES: CC(Oc1cnc2cc(N3CCOCC3)nc(O[C@H]3CC[C@@H](Nc4ncc(N5CCN(C)CC5)cn4)CC3)c2c1)c1cnc([N+](=O)[O-])n1C]

A mixture of 5-[4-[[5-(4-methylpiperazin-1-yl)pyrimidin-2-yl]amino]cyclohexoxy]-7-morpholino-1,6-naphthyridin-3-ol (50.mg, 0.1000 mmol), 5-(1-chloroethyl)-1-methyl-2-nitro-imidazole (20.03 mg, 0.1100 mmol) LFA009 and Potassium carbonate (33.18 mg, 0.2400 mmol) in DMF (1 mL) was stirred at RT for 21 h. The reaction mixture was diluted with EtOAc (20 mL) and washed with water (20 mL). The aqueous phase was extracted with 10% MeOH/EtOAc (2×20 mL). The combined organic phases were washed with brine (2×20 mL), dried (Na₂SO₄), filtered and concentrated in vacuo to give the crude product as an orange gum (61 mg). The crude product was purified by flash column chromatography (10 g silica column, eluting with 0-20% 2 M NH₃/MeOH in EtOAc) to give impure product as a yellow gum (47 mg) The yellow gum was further purified by basic prep. HPLC (method: basic prep.; 5-95% 0.005 M NH₄OH/MeCN in 0.005 M NH₄OH/H2O) to give the product, N-[4-[[3-[1-(3-methyl-2-nitro-imidazol-4-yl)ethoxy]-7-morpholino-1,6-naphthyridin-5-yl]oxy]cyclohexyl]-5-(4-methylpiperazin-1-yl)pyrimidin-2-amine (26 mg, 40% yield) as a yellow solid.

1H NMR (400 MHz, DMSO-d6) δ: 8.69 (d, J=3.0 Hz, 1H), 8.09 (s, 2H), 7.76 (dd, J=3.0, 0.7 Hz, 1H), 7.37 (s, 1H), 6.55 (d, J=8.0 Hz, 1H), 6.51 (d, J=0.8 Hz, 1H), 5.97 (q, J=6.4 Hz, 1H), 5.40-5.35 (m, 1H), 3.99 (s, 3H), 3.86-3.77 (m, 1H), 3.73 (dd, J=5.8, 3.9 Hz, 4H), 3.43 (dd, J=6.4, 3.5 Hz, 4H), 3.01-2.89 (m, 4H), 2.45-2.40 (m, 4H), 2.20 (s, 3H), 2.13-1.95 (m, 2H), 1.86-1.64 (m, 6H), 1.75 (d, J=6.5 Hz, 3H).

LCMS (long, acidic): 1.74 min, 674.3 [M+H]+

### N-[1-(1-methyl-5-nitro-pyrazol-4-yl)ethyl]-N-[5-[4-[[5-(4-methylpiperazin-1-yl)pyrimidin-2-yl]amino]cyclohexoxy]-7-morpholino-1,6-naphthyridin-3-yl]methanesulfonamide (LFA2067)

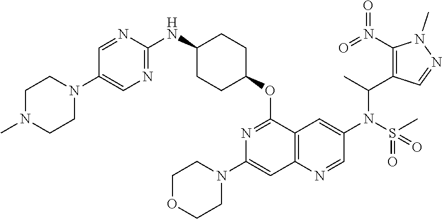 [SMILES: CC(c1cnn(C)c1[N+](=O)[O-])N(c1cnc2cc(N3CCOCC3)nc(O[C@H]3CC[C@@H](Nc4ncc(N5CCN(C)CC5)cn4)CC3)c2c1)S(C)(=O)=O]

A mixture of N-[5-[4-[[5-(4-methylpiperazin-1-yl)pyrimidin-2-yl]amino]cyclohexoxy]-7-morpholino-1,6-naphthyridin-3-yl]methanesulfonamide (30.mg, 0.0500 mmol), 4-(1-chloroethyl)-1-methyl-5-nitro-pyrazole (11.42 mg, 0.0600 mmol) and Potassium carbonate (17.34 mg, 0.1300 mmol) in DMF (1 mL) was heated to 100 C and stirred for 18 h. After cooling to RT, the mixture was diluted with EtOAc (20 mL) and washed with water (20 mL). The aqueous phase was extracted with 10% MeOH/EtOAc (2×20 mL). The combined organic phases were washed with brine (2×40 mL), dried (Na2SO4), filtered and concentrated in vacuo to give the crude product as an orange gum (37 mg). The crude product was purified by flash column chromatography (10 g silica column, eluting with 0-7% 2 M NH3/MeOH in DCM) to give impure product as a yellow gum/glass (13 mg). The impure product was further purified by basic prep. HPLC (method: basic prep.; 5-95% 0.005 M NH₄OH/MeCN in 0.005 M NH₄OH/H30) to give pure product as a yellow gum/glass (12 mg). The gum/glass was suspended in TBME (2 mL), the solvent removed in the Genevac and the sample dried in a vacuum oven at 50 C for 20 h to give the product, N-[1-(1-methyl-5-nitro-pyrazol-4-yl)ethyl]-N-[5-[4-[[5-(4-methylpiperazin-1-yl)pyrimidin-2-yl]amino]cyclohexoxy]-7-morpholino-1,6-naphthyridin-3-yl]methanesulfonamide (11 mg, 29% yield), as a yellow solid.

1H NMR (400 MHz, DMSO-d6) δ: 8.39 (d, J=2.5 Hz, 1H), 8.10 (s, 2H), 7.83 (d, J=2.5 Hz, 1H), 7.27 (s, 1H), 6.58-6.49 (m, 2H), 5.93 (q, J=7.0 Hz, 1H), 5.42-5.33 (m, 1H), 4.13 (s, 3H), 3.88-3.76 (m, 1H), 3.77-3.69 (m, 4H), 3.59-3.47 (m, 4H), 3.11 (s, 3H), 3.00-2.91 (m, 4H), 2.47-2.40 (m, 4H), 2.21 (s, 3H), 2.14-2.02 (m, 2H), 1.87-1.73 (m, 4H), 1.74-1.60 (m, 2H), 1.54 (d, J=7.0 Hz, 3H).

LCMS (long, acidic): 1.78 min, 751.2 [M+H]+

### N-[1-(3-methyl-2-nitro-imidazol-4-yl)ethyl]-N-[5-[4-[[5-(1-methylpyrrolidin-3-yl)oxypyrimidin-2-yl]amino]cyclohexoxy]-7-morpholino-1,6-naphthyridin-3-yl]methanesulfonamide (LFA2068)

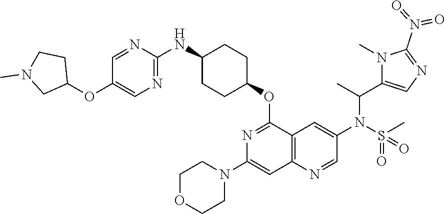 [SMILES: CC(c1cnc([N+](=O)[O-])n1C)N(c1cnc2cc(N3CCOCC3)nc(O[C@H]3CC[C@@H](Nc4ncc(OC5CCN(C)C5)cn4)CC3)c2c1)S(C)(=O)=O]

N-[5-[4-[[5-(1-methylpyrrolidin-3-yl)oxypyrimidin-2-yl]amino]cyclohexoxy]-7-morpholino-1,6-naphthyridin-3-yl]methanesulfonamide (40.mg, 0.0700 mmol), 5-(1-chloroethyl)-1-methyl-2-nitro-imidazole (13.93 mg, 0.0700 mmol) LFA009 and Potassium carbonate (27.7 mg, 0.2000 mmol) were combined in DMF (0.5000 mL) at room temperature and stirred under an argon atmosphere for 22 hours. The reaction mixture was filtered and diluted with DMSO. The sample was purified by basic prep HPLC. The solvent was removed from the appropriate fractions in the Genevac evaporator. The appropriate fractions were combined and the solvent removed at reduced pressure to afford N-[1-(3-methyl-2-nitro-imidazol-4-yl)ethyl]-N-[5-[4-[[5-(1-methylpyrrolidin-3-yl)oxypyrimidin-2-yl]amino]cyclohexoxy]-7-morpholino-1,6-naphthyridin-3-yl]methanesulfonamide (31 mg, 62% yield).

1H NMR (400 MHz, DMSO) δ: 8.35 (d, J=2.5 Hz, 1H), 8.06 (s, 2H), 7.72 (d, J=2.5 Hz, 1H), 6.80 (d, J=0.6 Hz, 1H), 6.70 (d, J=7.8 Hz, 1H), 6.50 (d, J=0.9 Hz, 1H), 5.72 (q, J=6.9 Hz, 1H), 5.31 (s, 1H), 4.79-4.71 (m, 1H), 4.17 (s, 3H), 3.86-3.65 (m, 5H), 3.62-3.43 (m, 4H), 3.29 (s, 3H), 2.75-2.54 (m, 4H), 2.37-2.29 (m, 1H), 2.24 (s, 4H), 2.14-2.04 (m, 1H), 1.97 (d, J=13.6 Hz, 1H), 1.87-1.53 (m, 9H).

LCMS (long, acidic): rt 1.712 min, 752.26 [M+H]+

### N-[4-[[3-[(3-methyl-2-nitro-imidazol-4-yl)methoxy]-7-morpholino-1,6-naphthyridin-5-yl]oxy]cyclohexyl]-5-(2-morpholinoethoxy)pyrimidin-2-amine (LFA2069)

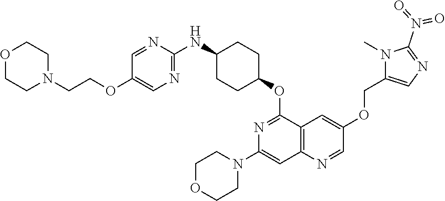 [SMILES: Cn1c(COc2cnc3cc(N4CCOCC4)nc(O[C@H]4CC[C@@H](Nc5ncc(OCCN6CCOCC6)cn5)CC4)c3c2)cnc1[N+](=O)[O-]]

7-morpholino-5-[4-[[5-(2-morpholinoethoxy)pyrimidin-2-yl]amino]cyclohexoxy]-1,6-naphthyridin-3-ol (47.mg, 0.0900 mmol), 5-(bromomethyl)-1-methyl-2-nitro-imidazole (28.12 mg, 0.1300 mmol) LFA007 and Potassium carbonate (35.33 mg, 0.2600 mmol) were transferred to a 10 mL RB flask and DMF (1 mL) added. The mixture was stirred at room temperature for 18 h. Reaction mixture filtered through a syringe filter and filtrate purified by basic prep HPLC (3 injections) and relevant fractions were combined and concentrated in vacuo to give 31 mg of a yellow solid. Further purified by acidic prep HPLC (2×300 uL injections in DMSO) and relevant fractions free based by passing down a 2 g SCX column, washing with MeOH and eluting the product off in 2M ammonia in MeOH. On concentration N-[4-[[3-[(3-methyl-2-nitro-imidazol-4-yl)methoxy]-7-morpholino-1,6-naphthyridin-5-yl]oxy]cyclohexyl]-5-(2-morpholinoethoxy)pyrimidin-2-amine (23.9 mg, 41% yield) was collected as a pale orange solid.

1H NMR (400 MHz, DMSO) δ: 8.69 (d, J=3.0 Hz, 1H), 8.12 (s, 2H), 7.84 (dd, J=3.0, 0.8 Hz, 1H), 7.39 (s, 1H), 6.68 (d, J=7.9 Hz, 1H), 6.54 (d, J=0.9 Hz, 1H), 5.44 (s, 2H), 5.42-5.37 (m, 1H), 4.05 (t, J=5.7 Hz, 2H), 4.01 (s, 3H), 3.88-3.78 (m, 1H), 3.78-3.70 (m, 4H), 3.60-3.53 (m, 4H), 3.49-3.40 (m, 4H), 2.63 (t, J=5.7 Hz, 2H), 2.47-2.41 (m, 4H), 2.14-2.01 (m, 2H), 1.85-1.71 (m, 6H).

LC-MS (long, acidic method) rt 1.74 min, 691.2 [M+H]+

### N-[1-(3-methyl-2-nitro-imidazol-4-yl)ethyl]-N-[5-[4-[[5-(4-methylpiperazin-1-yl)pyrimidin-2-yl]amino]cyclohexoxy]-7-morpholino-1,6-naphthyridin-3-yl]methanesulfonamide (LFA2070)

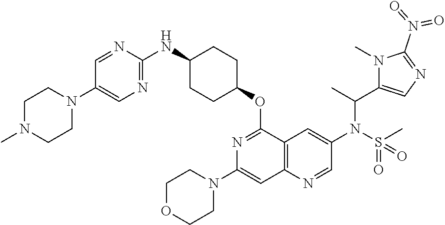 [SMILES: CC(c1cnc([N+](=O)[O-])n1C)N(c1cnc2cc(N3CCOCC3)nc(O[C@H]3CC[C@@H](Nc4ncc(N5CCN(C)CC5)cn4)CC3)c2c1)S(C)(=O)=O]

N-[5-[4-[[5-(4-methylpiperazin-1-yl)pyrimidin-2-yl]amino]cyclohexoxy]-7-morpholino-1,6-naphthyridin-3-yl]methanesulfonamide (40.mg, 0.0700 mmol), 5-(1-chloroethyl)-1-methyl-2-nitro-imidazole (13.96 mg, 0.0700 mmol) LFA009 and Potassium carbonate (27.75 mg, 0.2000 mmol) were combined in DMF (1 mL) at room temperature and stirred under an argon atmosphere for 22 hours. The reaction mixture was filtered and diluted with DMSO. The sample was purified by basic prep HPLC. The solvent was removed from the appropriate fractions in the Genevac evaporator to N-[1-(3-methyl-2-nitro-imidazol-4-yl)ethyl]-N-[5-[4-[[5-(4-methylpiperazin-1-yl)pyrimidin-2-yl]amino]cyclohexoxy]-7-morpholino-1,6-naphthyridin-3-yl]methanesulfonamide (19.1 mg, 38% yield).

1H NMR (400 MHz, MeOD) δ: 8.36 (d, J=2.6 Hz, 1H), 8.11 (s, 2H), 7.83 (d, J=2.4 Hz, 1H), 6.75 (d, J=0.7 Hz, 1H), 6.46 (d, J=0.9 Hz, 1H), 5.91-5.86 (m, 1H), 5.43-5.36 (m, 1H), 4.28 (s, 3H), 3.81 (t, J=4.9 Hz, 5H), 3.64-3.54 (m, 4H), 3.20 (s, 3H), 3.11-3.01 (m, 4H), 2.68-2.59 (m, 4H), 2.35 (s, 3H), 2.18-2.10 (m, 2H), 1.95-1.76 (m, 5H), 1.74 (d, J=6.9 Hz, 3H), 1.70-1.60 (m, 2H).

LCMS (long, acidic): rt 1.587 min, 751.2 [M+H]+

### N-methyl-N-[4-[[3-[(3-methyl-2-nitro-imidazol-4-yl)methoxy]-7-morpholino-1,6-naphthyridin-5-yl]oxy]cyclohexyl]pyrimidin-2-amine (LFA2071)

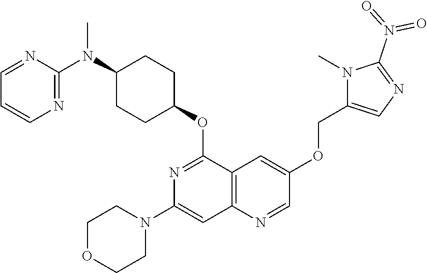 [SMILES: Cn1c(COc2cnc3cc(N4CCOCC4)nc(O[C@H]4CC[C@@H](N(C)c5ncccn5)CC4)c3c2)cnc1[N+](=O)[O-]]

A mixture of 5-[4-[methyl(pyrimidin-2-yl)amino]cyclohexoxy]-7-morpholino-1,6-naphthyridin-3-ol (50.mg, 0.1100 mmol), 5-(chloromethyl)-1-methyl-2-nitro-imidazole (24.13 mg, 0.1400 mmol) and Potassium carbonate (39.58 mg, 0.2900 mmol) in DMF (1 mL) was stirred at RT for 21 h. The reaction mixture was diluted with water (5 mL) and the resultant precipitate filtered and washed with water (2×3 mL). The collected solid was dried by drawing air through for 10 min. then washed through with DCM (30 mL). The DCM solution was concentrated in vacuo to give the crude product as an orange gummy solid (59 mg). The gummy solid was purified by basic prep. HPLC (method: basic prep.; 5-95% 0.005 M NH₄OH/MeCN in 0.005 M NH4OH/H2O) to give a yellow solid (36 mg). The solid was suspended in IPA (2 mL), sonicated and then heated to 45 C while spinning on a rotary evaporator for 10 min. The suspension was filtered, and the collected solid was washed with IPA (2×1 mL) and dried to give the product, N-methyl-N-[4-[[3-[(3-methyl-2-nitro-imidazol-4-yl)methoxy]-7-morpholino-1,6-naphthyridin-5-yl]oxy]cyclohexyl]pyrimidin-2-amine (31 mg, 47% yield), as a yellow solid.

1H NMR (400 MHz, DMSO-d6) δ: 8.70 (d, J=3.0 Hz, 1H), 8.35 (d, J=4.7 Hz, 2H), 7.90 (dd, J=3.0, 0.8 Hz, 1H), 7.38 (s, 1H), 6.60-6.55 (m, 2H), 5.51-5.42 (m, 3H), 4.85-4.69 (m, 1H), 4.01 (s, 3H), 3.79-3.70 (m, 4H), 3.50-3.40 (m, 4H), 3.06 (s, 3H), 2.28-2.17 (m, 2H), 2.13-1.98 (m, 2H), 1.88-1.75 (m, 2H), 1.63-1.47 (m, 2H).

LCMS (long, acidic): 2.38 min, 576.2 [M+H]; 98% purity @254 nm.

### N-[(1-methyl-5-nitro-pyrazol-4-yl)methyl]-N-[5-[4-[[5-(4-methylpiperazin-1-yl)pyrimidin-2-yl]amino]cyclohexoxy]-7-morpholino-1,6-naphthyridin-3-yl]methanesulfonamide (LFA2072)

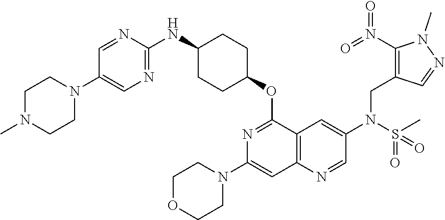 [SMILES: CN1CCN(c2cnc(N[C@H]3CC[C@@H](Oc4nc(N5CCOCC5)cc5ncc(N(Cc6cnn(C)c6[N+](=O)[O-])S(C)(=O)=O)cc45)CC3)nc2)CC1]

N-[5-[4-[[5-(4-methylpiperazin-1-yl)pyrimidin-2-yl]amino]cyclohexoxy]-7-morpholino-1,6-naphthyridin-3-yl]methanesulfonamide (57.5 mg, 0.1000 mmol) was transferred to a 10 mL RB flask and Potassium carbonate (39.89 mg, 0.2900 mmol) added followed by a solution of 4-(chloromethyl)-1-methyl-5-nitro-pyrazole (18.58 mg, 0.1100 mmol) in DMF (1 mL). The mixture was stirred under an argon atmosphere at room temperature for 16 h and then filtered through a syringe filter and washed through with DMSO (200 uL). Combined filtrate and washings purified by basic prep HPLC (3×400 uL) and relevant fractions were concentrated to dryness under reduced pressure to afford 14.4 mg of an orange solid. Further purified by acidic prep HPLC (1×400 uL injection in DMSO). Relevant fractions were free based using a 1 g SCX column by washing with MeOH and eluting the product off in 2M ammonia in MeOH. Relevant fractions were combined and concentrated to afford N-[(1-methyl-5-nitro-pyrazol-4-yl)methyl]-N-[5-[4-[[5-(4-methylpiperazin-1-yl)pyrimidin-2-yl]amino]cyclohexoxy]-7-morpholino-1,6-naphthyridin-3-yl]methanesulfonamide (16.1 mg, 23% yield) as an orange solid.

1H NMR (400 MHz, DMSO) δ: 8.79 (d, J=2.6 Hz, 1H), 8.20 (dd, J=2.6, 0.8 Hz, 1H), 8.10 (s, 2H), 7.72 (s, 1H), 6.52 (s, 1H), 6.49 (d, J=7.8 Hz, 1H), 5.40-5.33 (m, 1H), 5.11 (s, 2H), 4.05 (s, 3H), 3.88-3.77 (m, 1H), 3.76-3.69 (m, 4H), 3.54-3.47 (m, 4H), 3.22 (s, 3H), 3.01-2.93 (m, 4H), 2.49-2.41 (m, 4H), 2.22 (s, 3H), 2.12-2.01 (m, 2H), 1.84-1.68 (m, 6H).

LCMS (long, acidic): 1.74 min, 737.3 [M+H]+, 100% purity @ 254 nm

### N-[5-[4-[[5-[2-(dimethylamino)ethoxy]pyrimidin-2-yl]amino]cyclohexoxy]-7-morpholino-1,6-naphthyridin-3-yl]-N-[1-(1-methyl-5-nitro-pyrazol-4-yl)ethyl]methanesulfonamide (LFA2073)

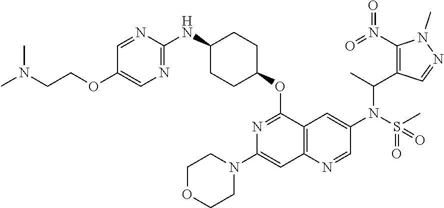 [SMILES: CC(c1cnn(C)c1[N+](=O)[O-])N(c1cnc2cc(N3CCOCC3)nc(O[C@H]3CC[C@@H](Nc4ncc(OCCN(C)C)cn4)CC3)c2c1)S(C)(=O)=O]

N-[5-[4-[[5-[2-(dimethylamino)ethoxy]pyrimidin-2-yl]amino]cyclohexoxy]-7-morpholino-1,6-naphthyridin-3-yl]methanesulfonamide (35.mg, 0.0600 mmol) was transferred to a 5 mL microwave vial and Potassium carbonate (24.73 mg, 0.1800 mmol) added followed by a solution of 4-(1-chloroethyl)-1-methyl-5-nitro-pyrazole (12.44 mg, 0.0700 mmol) in DMF (0.5000 mL). The mixture was heated at 100 C under an argon atmosphere for 15 h and then further 4-(1-chloroethyl)-1-methyl-5-nitro-pyrazole (6.22 mg, 0.0300 mmol) dissolved in DMF (0.1 mL) was added in one portion and the heating continued for 5 h. The mixture was cooled to room temperature and then filtered through a syringe filter and washed through with DMSO (200 uL). Combined filtrate and washings purified by basic prep HPLC (2×350 uL) and relevant fractions were concentrated to dryness under reduced pressure to afford solid. Further purified by acidic prep HPLC (1×400 uL injection in DMSO). Relevant fractions were free based using a 1 g SCX column by washing with MeOH and eluting the product off in 2M ammonia in MeOH. Relevant fractions were combined and concentrated to afford N-[5-[4-[[5-[2-(dimethylamino)ethoxy]pyrimidin-2-yl]amino]cyclohexoxy]-7-morpholino-1,6-naphthyridin-3-yl]-N-[1-(1-methyl-5-nitro-pyrazol-4-yl)ethyl]methanesulfonamide (5.8 mg, 13% yield) as an orange solid.

1H NMR (400 MHz, DMSO) δ: 8.39 (d, J=2.5 Hz, 1H), 8.12 (s, 2H), 7.83 (d, J=2.5 Hz, 1H), 7.27 (s, 1H), 6.67 (d, J=7.8 Hz, 1H), 6.54 (s, 1H), 5.93 (q, J=7.0 Hz, 1H), 5.39-5.35 (m, 1H), 4.13 (s, 3H), 4.01 (t, J=5.8 Hz, 2H), 3.87-3.78 (m, 1H), 3.77-3.70 (m, 4H), 3.56-3.50 (m, 4H), 3.11 (s, 3H), 2.56 (t, J=5.8 Hz, 2H), 2.19 (s, 6H), 2.14-2.01 (m, 2H), 1.86-1.60 (m, 6H), 1.54 (d, J=7.0 Hz, 3H).

LCMS (long, acidic): 1.76 min, 740.3 [M+H]+, 99.3% purity @ 254 nm

### N-[1-(3-methyl-2-nitro-imidazol-4-yl)ethyl]-N-[7-morpholino-5-[4-[[5-(2-morpholinoethyl)pyrimidin-2-yl]amino]cyclohexoxy]-1,6-naphthyridin-3-yl]methanesulfonamide (LFA2074)

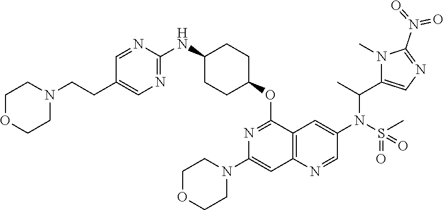 [SMILES: CC(c1cnc([N+](=O)[O-])n1C)N(c1cnc2cc(N3CCOCC3)nc(O[C@H]3CC[C@@H](Nc4ncc(CCN5CCOCC5)cn4)CC3)c2c1)S(C)(=O)=O]

A mixture of N-[7-morpholino-5-[4-[[5-(2-morpholinoethyl)pyrimidin-2-yl]amino]cyclohexoxy]-1,6-naphthyridin-3-yl]methanesulfonamide (61.mg, 0.1000 mmol), 5-(1-chloroethyl)-1-methyl-2-nitro-imidazole (22.65 mg, 0.1200 mmol) and Potassium carbonate (34.4 mg, 0.2500 mmol) in DMF (1 mL) was stirred at RT for 72 h. Water (5 mL) was added and the resultant precipitate was filtered, washed with water (3×2 mL) and dried to give the crude product as a brown solid (25 mg). The brown solid was purified by basic prep. HPLC (method: basic prep.; 5-95% 0.005 M NH₄OH/MeCN in 0.005 M NH₄OH/H2O) to give the product, N-[1-(3-methyl-2-nitro-imidazol-4-yl)ethyl]-N-[7-morpholino-5-[4-[[5-(2-morpholinoethyl)pyrimidin-2-yl]amino]cyclohexoxy]-1,6-naphthyridin-3-yl]methanesulfonamide (11 mg, 14% yield), as a yellow solid.

1H NMR (400 MHz, DMSO-d6) δ: 8.35 (d, J=2.5 Hz, 1H), 8.16 (s, 2H), 7.72 (s, 1H), 6.88 (d, J=7.9 Hz, 1H), 6.80 (d, J=0.6 Hz, 1H), 6.50 (d, J=0.9 Hz, 1H), 5.72 (q, J=6.8 Hz, 1H), 5.34-5.27 (m, 1H), 4.17 (s, 3H), 3.90-3.78 (m, 1H), 3.76-3.68 (m, 4H), 3.60-3.54 (m, 4H), 3.54-3.49 (m, 4H), 3.30 (s, 3H), 2.56-2.51 (m, 2H), 2.46-2.35 (m, 6H), 2.17-2.05 (m, 1H), 2.05-1.90 (m, 1H), 1.85-1.70 (m, 4H), 1.65 (d, J=6.9 Hz, 3H), 1.69-1.55 (m, 2H).

LCMS (long, acidic): 1.70 min, 766.2 [M+H]; 99% purity @254 nm

### N-[5-[4-[[5-[2-(dimethylamino)ethoxy]pyrimidin-2-yl]amino]cyclohexoxy]-7-morpholino-1,6-naphthyridin-3-yl]-N-[(1-methyl-5-nitro-pyrazol-4-yl)methyl]methanesulfonamide (LFA2075)

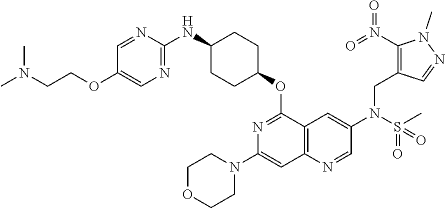 [SMILES: CN(C)CCOc1cnc(N[C@H]2CC[C@@H](Oc3nc(N4CCOCC4)cc4ncc(N(Cc5cnn(C)c5[N+](=O)[O-])S(C)(=O)=O)cc34)CC2)nc1]

N-[5-[4-[[5-[2-(dimethylamino)ethoxy]pyrimidin-2-yl]amino]cyclohexoxy]-7-morpholino-1,6-naphthyridin-3-yl]methanesulfonamide (25.9 mg, 0.0400 mmol) was transferred to a 5 mL microwave vial and Potassium carbonate (18.3 mg, 0.1300 mmol) added followed by a solution of 4-(chloromethyl)-1-methyl-5-nitro-pyrazole (8.53 mg, 0.0500 mmol) in DMF (0.4000 mL). The mixture was stirred under an argon atmosphere at room temperature for 16 h and then filtered through a syringe filter and washed through with DMSO (200 uL). Combined filtrate and washings purified by basic prep HPLC (1×600 uL) and relevant fractions were concentrated to dryness under reduced pressure to afford crude product as a yellow gum. Further purified by acidic prep HPLC (1×500 uL injection in DMSO). Relevant fractions were combined and free based by loading onto a 1 g SCX column, washing with MeOH and eluting the product off with 2M ammonia in MeOH. On concentration N-[5-[4-[[5-[2-(dimethylamino)ethoxy]pyrimidin-2-yl]amino]cyclohexoxy]-7-morpholino-1,6-naphthyridin-3-yl]-N-[(1-methyl-5-nitro-pyrazol-4-yl)methyl]methanesulfonamide (4.2 mg, 13% yield) was collected as a yellow solid.

1H NMR (400 MHz, DMSO) δ: 8.79 (d, J=2.6 Hz, 1H), 8.20 (dd, J=2.7, 0.8 Hz, 1H), 8.12 (s, 2H), 7.72 (s, 1H), 6.60 (d, J=7.7 Hz, 1H), 6.52 (s, 1H), 5.41-5.33 (m, 1H), 5.11 (s, 2H), 4.05 (s, 3H), 4.02 (t, J=5.8 Hz, 2H), 3.87-3.76 (m, 1H), 3.76-3.69 (m, 4H), 3.54-3.47 (m, 4H), 3.22 (s, 3H), 2.59 (t, J=5.8 Hz, 2H), 2.21 (s, 6H), 2.13-2.01 (m, 2H), 1.84-1.66 (m, 6H).

LCMS (long, acidic): 1.75 min, 726.2 [M+H]+, 100% purity @ 254 nm

### N-[(1-methyl-5-nitro-pyrazol-4-yl)methyl]-N-[7-morpholino-5-[4-[[5-(2-morpholinoethoxy)pyrimidin-2-yl]amino]cyclohexoxy]-1,6-naphthyridin-3-yl]methanesulfonamide (LFA2076)

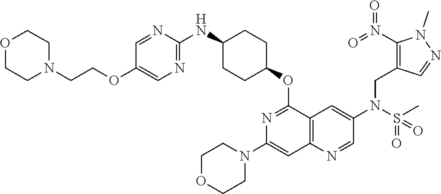 [SMILES: Cn1ncc(CN(c2cnc3cc(N4CCOCC4)nc(O[C@H]4CC[C@@H](Nc5ncc(OCCN6CCOCC6)cn5)CC4)c3c2)S(C)(=O)=O)c1[N+](=O)[O-]]

N-[7-morpholino-5-[4-[[5-(2-morpholinoethoxy)pyrimidin-2-yl]amino]cyclohexoxy]-1,6-naphthyridin-3-yl]methanesulfonamide (40.mg, 0.0600 mmol), 4-(chloromethyl)-1-methyl-5-nitro-pyrazole (12.29 mg, 0.0700 mmol) and potassium carbonate (26.38 mg, 0.1900 mmol) were combined in DMF (1 mL) and stirred at room temperature. The reaction was filtered through a syringe filter and washed with DMSO. The solution was purified by basic prep HPLC. The appropriate fractions were combined and the solvent removed in the Genevac to afford which was further purified by acidic prep HPLC. The appropriate fractions were loaded onto a 2 g SCX. The column was washed with methanol and eluted with 2M ammonia in methanol. The ammonia fraction was concentrated at reduced pressure to afford N-[(1-methyl-5-nitro-pyrazol-4-yl)methyl]-N-[7-morpholino-5-[4-[[5-(2-morpholinoethoxy)pyrimidin-2-yl]amino]cyclohexoxy]-1,6-naphthyridin-3-yl]methanesulfonamide (15.5 mg, 32% yield)

1H NMR (400 MHz, DMSO) δ: 8.79 (d, J=2.6 Hz, 1H), 8.20 (dd, J=2.6, 0.8 Hz, 1H), 8.13 (s, 2H), 7.72 (s, 1H), 6.62 (d, J=7.8 Hz, 1H), 6.53 (d, J=0.8 Hz, 1H), 5.37 (s, 1H), 5.11 (s, 2H), 4.09-4.01 (m, 5H), 3.81 (s, 1H), 3.76-3.68 (m, 4H), 3.60-3.54 (m, 4H), 3.51 (t, J=4.9 Hz, 4H), 3.22 (s, 3H), 2.63 (t, J=5.7 Hz, 2H), 2.45 (d, J=5.1 Hz, 4H), 2.12-2.00 (m, 2H), 1.83-1.69 (m, 6H).

LC-MS (long, acidic method) rt 1.746 min, 768.2 [M+H]+

### N-[1-(1-methyl-5-nitro-pyrazol-4-yl)ethyl]-N-[7-morpholino-5-[4-[[5-(2-morpholinoethoxy)pyrimidin-2-yl]amino]cyclohexoxy]-1,6-naphthyridin-3-yl]methanesulfonamide (LFA2077)

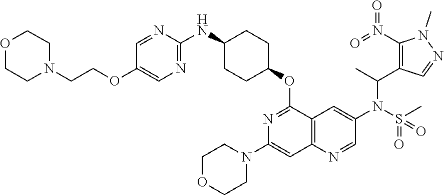 [SMILES: CC(c1cnn(C)c1[N+](=O)[O-])N(c1cnc2cc(N3CCOCC3)nc(O[C@H]3CC[C@@H](Nc4ncc(OCCN5CCOCC5)cn4)CC3)c2c1)S(C)(=O)=O]

N-[7-morpholino-5-[4-[[5-(2-morpholinoethoxy)pyrimidin-2-yl]amino]cyclohexoxy]-1,6-naphthyridin-3-yl]methanesulfonamide (40.mg, 0.0600 mmol), 4-(1-chloroethyl)-1-methyl-5-nitro-pyrazole (13.27 mg, 0.0700 mmol) and potassium carbonate (26.38 mg, 0.1900 mmol) were combined in DMF (1 mL) and heated to 100 C for 17 hours. The reaction was allowed to cool to room temperature, filtered through a syringe filter and washed with DMSO. The solution was purified by basic prep HPLC. The appropriate fractions were combined and the solvent removed in the Genevac to afford product. Further purified by acidic prep HPLC. The appropriate fractions were loaded onto a 2 g SCX. The column was washed with methanol and eluted with 2M ammonia in methanol. The ammonia fraction was concentrated at reduced pressure to afford N-[1-(1-methyl-5-nitro-pyrazol-4-yl)ethyl]-N-[7-morpholino-5-[4-[[5-(2-morpholinoethoxy)pyrimidin-2-yl]amino]cyclohexoxy]-1,6-naphthyridin-3-yl]methanesulfonamide (7.9 mg, 16% yield)

1H NMR (400 MHz, DMSO) δ: 8.39 (d, J=2.6 Hz, 1H), 8.13 (s, 2H), 7.83 (d, J=2.5 Hz, 1H), 7.27 (s, 1H), 6.69 (d, J=7.8 Hz, 1H), 6.54 (d, J=0.9 Hz, 1H), 5.93 (q, J=7.0 Hz, 1H), 5.39-5.35 (m, 1H), 4.13 (s, 3H), 4.06 (t, J=5.7 Hz, 2H), 3.81 (s, 1H), 3.73 (t, J=4.8 Hz, 4H), 3.65-3.45 (m, 8H), 3.11 (s, 3H), 2.64 (s, 2H), 2.45 (s, 4H), 2.11-2.02 (m, 2H), 1.83-1.61 (m, 6H), 1.53 (d, J=7.0 Hz, 3H).

LC-MS (long, acidic method) rt 1.791 min, 782.2 [M+H]+

### 5-[4-[(6-methyl-7,8-dihydro-5H-pyrido[4,3-d]pyrimidin-2-yl)amino]cyclohexoxy]-7-morpholino-1,6-naphthyridin-3-ol (LFA2078)

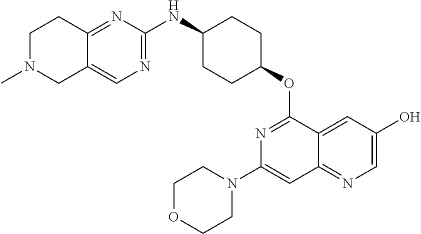 [SMILES: CN1CCc2nc(N[C@H]3CC[C@@H](Oc4nc(N5CCOCC5)cc5ncc(O)cc45)CC3)ncc2C1]

N-[4-[(3-methoxy-7-morpholino-1,6-naphthyridin-5-yl)oxy]cyclohexyl]-6-methyl-7,8-dihydro-5H-pyrido[4,3-d]pyrimidin-2-amine (60.mg, 0.1200 mmol), 5 M Lithium chloride (aq.) (0.47 mL, 2.37 mmol) and NMP (2 mL) were charged to a microwave vial. The vial was sealed and the mixture was stirred in a microwave reactor at 200 C for 2 h. Still SM present, heated in the microwave for a further 5 h at 200 C. After cooling to RT, the mixture was diluted with water (20 mL) and extracted with 5% MeOH/EtOAc (3×20 mL). The combined organic phases were dried (phase-separator) and concentrated in vacuo. The residue was re-dissolved in 5% AcOH/MeOH (2 mL) and loaded directly onto an SCX column (2 g). The column was flushed with MeOH (100 mL) followed by 2 M NH3/MeOH (50 mL). The NH3/MeOH fraction was concentrated in vacuo to give the crude product as an orange/brown gum. The orange/brown gum was purified by flash column chromatography (10 g Biotage silica column, eluting with 0-100% 2 M NH3/MeOH in DCM) to give an orange-brown solid. The residue required further purification by acidic prep HPLC (dissolved in 400 μL DMSO), the relevant fractions were combined and free based by loading onto a 2 g SCX cartridge, washing with MeOH and eluting the product off with 2M ammonia in MeOH. Relevant fractions were combined and concentrated in vacuo to give a yellow solid. Purified further by Basic prep HPLC and the relevant fractions concentrated in vacuo to give a yellow solid, 5-[4-[(6-methyl-7,8-dihydro-5H-pyrido[4,3-d]pyrimidin-2-yl)amino]cyclohexoxy]-7-morpholino-1,6-naphthyridin-3-ol (2.1 mg, 4% yield).

1H NMR (400 MHz, MeOD) δ: 8.47 (d, J=2.9 Hz, 1H), 8.02 (s, 1H), 7.79-7.74 (m, 1H), 6.46 (d, J=0.8 Hz, 1H), 5.44 (s, 1H), 3.87-3.80 (m, 4H), 3.51-3.44 (m, 6H), 2.86-2.76 (m, 4H), 2.47 (s, 3H), 2.20 (d, J=11.3 Hz, 2H), 1.97-1.89 (m, 3H), 1.89-1.75 (m, 4H).

LCMS (long, acidic): 1.403 min, 492 [M+H]+

### 5-[4-[[5-[2-(dimethylamino)ethoxy]pyrimidin-2-yl]amino]cyclohexoxy]-7-morpholino-1,6-naphthyridin-3-ol (LFA2079)

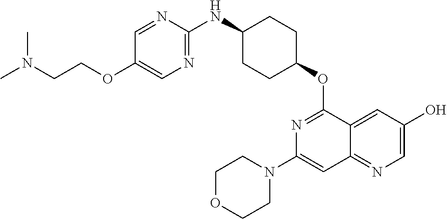 [SMILES: CN(C)CCOc1cnc(N[C@H]2CC[C@@H](Oc3nc(N4CCOCC4)cc4ncc(O)cc34)CC2)nc1]

A mixture of 5-[2-(dimethylamino)ethoxy]-N-[4-[(3-methoxy-7-morpholino-1,6-naphthyridin-5-yl)oxy]cyclohexyl]pyrimidin-2-amine (30.mg, 0.0600 mmol) and Lithium chloride (24.29 mg, 0.5700 mmol) in DMF (1.5 mL) was heated to 120 C and stirred for 18 h. A further portion of Lithium chloride (24.29 mg, 0.5700 mmol) was added and the mixture was stirred in the microwave at 160 C for 2 h, then at 180 C for 2 h. A further portion of Lithium chloride (24.29 mg, 0.5700 mmol) was added and the mixture was stirred in the microwave at 200 C for 2 h. After cooling to RT, the mixture was diluted with EtOAc (20 mL) and washed with water (20 mL). The aqueous phase was extracted with 5% MeOH/EtOAc (2×20 mL) and the combined organic phases were dried (phase-separator) and concentrated in vacuo to give the crude product as an orange gum (34 mg). The orange gum was purified by flash column chromatography (12 g Redisep Gold silica column, eluting with 0.5-20% 2 M NH₃/MeOH in DCM) to give impure product as an orange gum (10 mg) (note 1). The orange gum was further purified by basic prep. HPLC (method: basic prep.; 5-95% 0.005 M NH₄OH/MeCN in 0.005 M NH₄OH/H2O) to give the product, 5-[4-[[5-[2-(dimethylamino)ethoxy]pyrimidin-2-yl]amino]cyclohexoxy]-7-morpholino-1,6-naphthyridin-3-ol (5 mg, 17% yield), as a yellow solid.

1H NMR (400 MHz, DMSO-d6) δ: 8.51 (d, J=2.9 Hz, 1H), 8.11 (s, 2H), 7.57 (d, J=2.8 Hz, 1H), 6.76 (d, J=7.7 Hz, 1H), 6.47 (d, J=0.9 Hz, 1H), 5.40-5.25 (m, 1H), 4.00 (t, J=5.8 Hz, 2H), 3.81-3.68 (m, 5H), 3.40-3.35 (m, 4H)*, 2.56 (t, J=5.8 Hz, 2H), 2.19 (s, 6H), 2.16-2.01 (m, 2H), 1.84-1.66 (m, 6H). OH not directly observed. * partially overlaps with HO peak.2

LCMS (long, acidic): 1.48 min, 510.3 [M+H]+

### 5-[2-(dimethylamino)ethoxy]-N-[4-[(3-methoxy-7-morpholino-1,6-naphthyridin-5-yl)oxy]cyclohexyl]pyrimidin-2-amine

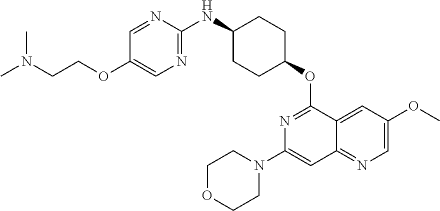

2-(2-chloropyrimidin-5-yl)oxy-N,N-dimethyl-ethanamine (98.73 mg, 0.4900 mmol), tBuBrettPhos-Pd-G3 (41.84 mg, 0.0500 mmol) and Potassium tert-butoxide (109.88 mg, 0.9800 mmol) were charged to a 2-5 mL microwave vial. The vial was sealed and purged with a stream of argon for 5 min. 4-[(3-methoxy-7-morpholino-1,6-naphthyridin-5-yl)oxy]cyclohexanamine (117.mg, 0.3300 mmol) in 1,4-dioxane (3 mL) was added and the mixture was degassed by sparging with a stream of argon for 5 min. The mixture was heated to 100-C and stirred for 18 h. After cooling to RT, the mixture was diluted with EtOAc (1 mL) and filtered through a pad of Celite, eluting with EtOAc (50 mL). The filtrate was concentrated in vacuo to give the crude product as a yellow gum (238 mg). The gum was purified by flash column chromatography (24 g silica Redisep Gold column, eluting with 0-6% 2 M NH/MeOH in DCM) to give the product, 35-[2-(dimethylamino)ethoxy]-N-[4-[(3-methoxy-7-morpholino-1,6-naphthyridin-5-yl)oxy]cyclohexyl]pyrimidin-2-amine (117 mg, 68% yield), as a yellow solid.

1H NMR (400 MHz, Methanol-d4) δ: 8.53 (d, J=3.0 Hz, 1H), 8.11 (s, 2H), 7.77 (dd, J=3.0, 0.8 Hz, 1H), 6.49 (d, J=0.8 Hz, 1H), 5.48-5.40 (m, 1H), 4.08 (dd, J=5.6, 5.1 Hz, 2H), 3.95 (s, 3H), 3.91-3.80 (m, 5H), 3.52-3.46 (m, 4H), 2.73 (t, J=5.4 Hz, 2H), 2.33 (s, 6H), 2.26-2.14 (m, 2H), 2.00-1.74 (m, 6H). NH not directly observed.

LCMS ( ) (short, acidic): 1.29 min, 524.3 [M+H]+

### 2-(2-chloropyrimidin-5-yl)oxy-N,N-dimethyl-ethanamine

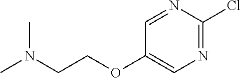

2-chloropyrimidin-5-ol (0.97 g, 7.43 mmol), 2-chloro-N,N-dimethyl-ethanamine; hydrochloride (3.21 g, 22.29 mmol), Cesium carbonate (7.26 g, 22.29 mmol) and Potassium iodide (1.23 g, 7.43 mmol) was stirred in DMF (15 mL) and MeCN (15 mL) under an argon atmosphere. The reaction was heated to 90 C for 5 hours and then cooled to room temperature, diluted with DCM (50 mL), washed with water (100 mL) and the aqueous layer extracted with further DCM (2×50 mL). The combined organics was dried (hydrophobic frit) and concentrated at reduced pressure. The resulting residue containing DMF was loaded onto a 10 g SCX, the column washed with methanol and eluted with 2Mammonia in methanol. The ammonia fraction was concentrated at reduced pressure to afford 2-(2-chloropyrimidin-5-yl)oxy-N,N-dimethyl-ethanamine (884 mg, 59% yield) as an orange/brown solid.

1H NMR (400 MHz, CDCl3) δ: 8.32 (s, 2H), 4.14 (t, J=5.5 Hz, 2H), 2.75 (t, J=5.5 Hz, 2H), 2.34 (s, 6H).

### 4-[(3-methoxy-7-morpholino-1,6-naphthyridin-5-yl)oxy]cyclohexanamine

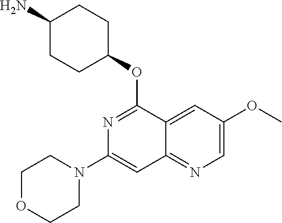 [SMILES: COc1cnc2cc(N3CCOCC3)nc(O[C@H]3CC[C@@H](N)CC3)c2c1]

Hydrogen chloride (3.7-4.3 M in 1,4-dioxane) (0.17 mL, 0.6300 mmol) was added to a stirred solution of tert-butyl N-[4-[(3-methoxy-7-morpholino-1,6-naphthyridin-5-yl)oxy]cyclohexyl]carbamate (29.mg, 0.0600 mmol) in MeOH (2.5 mL). The mixture was stirred at RT for 18 h. The mixture was concentrated in vacuo, and the residue was re-dissolved in MeOH (1 mL) and loaded directly onto an SCX column (0.5 g). The column was flushed with MeOH (80 mL) followed by 2 M NH3/MeOH (50 mL). The NH3/MeOH fraction was concentrated in vacuo to give the product, 4-[(3-methoxy-7-morpholino-1,6-naphthyridin-5-yl)oxy]cyclohexanamine (20 mg, 88% yield), as a yellow glassy solid.

1H NMR (400 MHz, Methanol-d4)—8.52 (d, J=3.0 Hz, 1H), 7.76 (dd, J=3.0, 0.8 Hz, 1H), 6.48 (d, J=0.9 Hz, 1H), 5.53-5.36 (m, 1H), 3.95 (s, 3H), 3.90-3.75 (m, 4H), 3.58-3.42 (m, 4H), 2.91-2.77 (m, 1H), 2.26-2.14 (m, 2H), 1.97-1.53 (m, 6H).

LCMS (short, acidic): 1.18 min, 359.3 [M+H]+

### tert-butylN-[4-[(3-methoxy-7-morpholino-1,6-naphthyridin-5-yl)oxy]cyclohexyl]carbamate

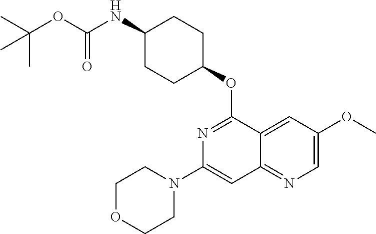 [SMILES: COc1cnc2cc(N3CCOCC3)nc(O[C@H]3CC[C@@H](NC(=O)OC(C)(C)C)CC3)c2c1]

tert-butyl N-[4-[(3-iodo-7-morpholino-1,6-naphthyridin-5-yl)oxy]cyclohexyl]carbamate (LFA019) (250.mg, 0.4500 mmol), Copper(I) iodide (8.59 mg, 0.0500 mmol), 1,10-phenanthroline (16.25 mg, 0.0900 mmol) and Cesium carbonate (293.84 mg, 0.9000 mmol) were charged to a 20 mL microwave vial. The vial was sealed and the mixture was purged with argon for 5 min, Anhydrous MeOH (5 mL) was added and the mixture was degassed by sparging with a stream of argon for 5 min. The mixture was heated to 100-C and stirred for 18 h. The mixture was concentrated in vacuo, and the residue was suspended in EtOAc (5 mL) and filtered through a pad of Celite, eluting with EtOAc (50 mL). The filtrate was concentrated in vacuo to give the crude product as a yellow gum/oil (266 mg). The gummy solid was purified by flash column chromatography (25 g silica column, eluting with 0-60% EtOAc in n-heptane) to give the product, tert-butylN-[4-[(3-methoxy-7-morpholino-1,6-naphthyridin-5-yl)oxy]cyclohexyl]carbamate (162 mg, 78% yield), as a yellow solid.

1H NMR (400 MHz, Methanol-d4) δ: 8.49 (d, J=3.0 Hz, 1H), 7.69 (dd, J=3.0, 0.8 Hz, 1H), 6.70 (d, J=6.9 Hz, 1H), 6.44 (d, J=0.8 Hz, 1H), 5.45-5.35 (m, 1H), 3.92 (s, 3H), 3.86-3.78 (m, 4H), 3.56-3.48 (m, 1H), 3.49-3.41 (m, 4H), 2.24-2.09 (m, 2H), 1.86-1.68 (m, 6H), 1.44 (s, 9H).

LCMS ( ) (short, acidic): 1.82 min, 459.3 [M+H]+

### 5-[4-[[5-(4-methylpiperazin-1-yl) pyrimidin-2-yl]amino]cyclohexoxy]-7-morpholino-1,6-naphthyridin-3-ol (LFA2080)

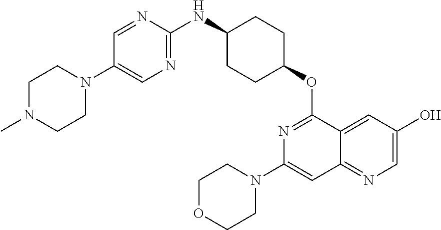 [SMILES: CN1CCN(c2cnc(N[C@H]3CC[C@@H](Oc4nc(N5CCOCC5)cc5ncc(O)cc45)CC3)nc2)CC1]

5-[4-[(5-bromopyrimidin-2-yl)amino]cyclohexoxy]-7-morpholino-1,6-naphthyridin-3-ol (91.mg, 0.1800 mmol), 1-methylpiperazine (0.04 mL, 0.3600 mmol), Tris(dibenzylideneacetone)dipalladium(0) (16.62 mg, 0.0200 mmol), JohnPhos (5.42 mg, 0.0200 mmol) and Sodium tert-butoxide (52.33 mg, 0.5400 mmol) were charged to a 2-5 mL microwave vial. The vial was sealed and the mixture was purged with a stream of argon for 5 min, 1,4-dioxane was added and the mixture was sparged with a stream of argon for 5 min. The mixture was heated to 100-C and stirred for 18 h. Further portions of Tris(dibenzylideneacetone)dipalladium(0) (16.62 mg, 0.0200 mmol), JohnPhos (5.42 mg, 0.0200 mmol) and 1-methylpiperazine (0.04 mL, 0.3600 mmol) were added and the vial re-sealed. The mixture was sparged with a stream of argon for 5 min. The mixture was heated to 100-C and stirred for 20 h. After cooling to RT, the mixture was diluted with EtOAc (2 mL) and filtered through a pad of Celite, eluting with EtOAc (50 mL). The filtrate was concentrated in vacuo to give the crude product as an orange/brown gum (151 mg). The orange/brown gum was purified by flash column chromatography (10 g silica column, eluting with 0-10% 2 M NH3/MeOH in DCM) to give impure product as an orange/yellow gummy solid (33 mg).3The impure product was further purified by basic prep. HPLC (method: basic prep.; 5-95% 0.005 M NH₄OH/MeCN in 0.005 M NH₄OH/H2O) to give the product, 5-[4-[[5-(4-methylpiperazin-1-yl)pyrimidin-2-yl]amino]cyclohexoxy]-7-morpholino-1,6-naphthyridin-3-ol (21 mg, 22% yield), as a yellow solid.

1H NMR (400 MHz, DMSO-d6) δ: 9.99 (s, 1H), 8.53 (d, J=2.9 Hz, 1H), 8.09 (s, 2H), 7.62 (dd, J=2.9, 0.8 Hz, 1H), 6.60 (d, J=7.6 Hz, 1H), 6.48 (d, J=0.8 Hz, 1H), 5.38-5.27 (m, 1H), 3.86-3.64 (m, 5H), 3.39 (dd, J=5.9, 3.9 Hz, 4H), 3.03-2.90 (m, 4H), 2.47-2.40 (m, 4H), 2.21 (s, 3H), 2.12-2.00 (m, 2H), 1.90-1.57 (m, 6H).

LCMS (long, acidic): 1.47 min, 521.3 [M+H]+

### 5-[4-[(5-bromopyrimidin-2-yl)amino]cyclohexoxy]-7-morpholino-1,6-naphthyridin-3-ol

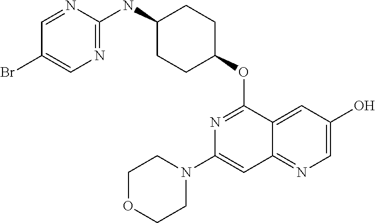

Triethylamine (0.36 mL, 2.58 mmol) was added to a stirred solution of 5-(4-aminocyclohexoxy)-7-morpholino-1,6-naphthyridin-3-ol (296.mg, 0.8600 mmol) and 5-bromo-2-chloro-pyrimidine (249.36 mg, 1.29 mmol) in EtOH (7 mL). The mixture was heated to 80-C and stirred for 18 h. The mixture was concentrated in vacuo and the residue was purified by flash column chromatography (25 g silica column, eluting with 0-5% MeOH in DCM) to give the product, 5-[4-[(5-bromopyrimidin-2-yl)amino]cyclohexoxy]-7-morpholino-1,6-naphthyridin-3-ol (110 mg, 25% yield) as a yellow/orange solid.

1H NMR (400 MHz, DMSO-d6) δ:10.02 (br. s, 1H), 8.54 (d, J=2.9 Hz, 1H), 8.37 (s, 2H), 7.62 (dd, J=2.9, 0.8 Hz, 1H), 7.50 (d, J=7.7 Hz, 1H), 6.49 (d, J=0.9 Hz, 1H), 5.39-5.29 (m, 1H), 3.86-3.78 (m, 1H), 3.74 (dd, J=5.9, 3.8 Hz, 4H), 3.40 (dd, J=5.6, 4.0 Hz, 4H), 2.15-2.05 (m, 2H), 1.84-1.67 (m, 6H).

LCMS (short, acidic): 1.59 min, 501.1/503.1 [M+H]+

### 4,6-dimethyl-N-[4-[[3-[(3-methyl-2-nitro-imidazol-4-yl)methoxy]-7-morpholino-1,6-naphthyridin-5-yl]oxy]cyclohexyl]pyrimidin-2-amine (LFA2081)

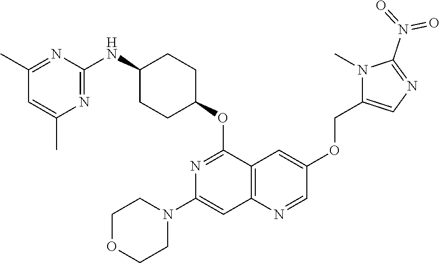 [SMILES: Cc1cc(C)nc(N[C@H]2CC[C@@H](Oc3nc(N4CCOCC4)cc4ncc(OCc5cnc([N+](=O)[O-])n5C)cc34)CC2)n1]

5-[4-[(4,6-dimethylpyrimidin-2-yl)amino]cyclohexoxy]-7-morpholino-1,6-naphthyridin-3-ol (50.mg, 0.1100 mmol), 5-(chloromethyl)-1-methyl-2-nitro-imidazole (23.38 mg, 0.1300 mmol) and Potassium carbonate (53.69 mg, 0.3900 mmol) were transferred to a 10 mL RB flask and stirred at ambient temperature under an argon atmosphere. DMF (1 mL) was added and the mixture stirred at room temperature for 16 h. Sample filtered through a syringe filter, washed through with DMSO and combined filtrate and washings purified by basic prep HPLC (4×300 uL) injections. Relevant fractions were combined and concentrated to dryness in vacuo to afford a yellow solid yield, which was triturated with IPA (2 mL) and left standing over the weekend and resultant solid filtered off and dried under vacuum to afford a yellow solid. Combined with above solid and further dried in a vacuum oven at 40 C for 3 h to afford 4,6-dimethyl-N-[4-[[3-[(3-methyl-2-nitro-imidazol-4-yl)methoxy]-7-morpholino-1,6-naphthyridin-5-yl]oxy]cyclohexyl]pyrimidin-2-amine (23 mg, 35% yield) as a yellow solid.

1H NMR (400 MHz, DMSO) δ: 8.69 (d, J=3.0 Hz, 1H), 7.84 (d, J=3.0 Hz, 1H), 7.40 (s, 1H), 6.82 (d, J=8.1 Hz, 1H), 6.54 (s, 1H), 6.32 (s, 1H), 5.45 (s, 2H), 5.43-5.38 (m, 1H), 4.01 (s, 3H), 3.97-3.87 (m, 1H), 3.80-3.69 (m, 4H), 3.48-3.41 (m, 4H), 2.18 (s, 6H), 2.12-2.00 (m, 2H), 1.87-1.68 (m, 6H).

LC-MS (long, acidic method) rt 2.06 min, 590.2 [M+H]+

### 4,6-dimethyl-N-[4-[[3-[1-(3-methyl-2-nitro-imidazol-4-yl)ethoxy]-7-morpholino-1,6-naphthyridin-5-yl]oxy]cyclohexyl]pyrimidin-2-amine (LFA2082)

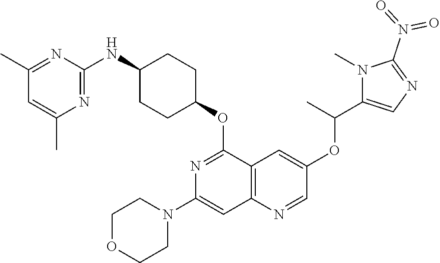 [SMILES: Cc1cc(C)nc(N[C@H]2CC[C@@H](Oc3nc(N4CCOCC4)cc4ncc(OC(C)c5cnc([N+](=O)[O-])n5C)cc34)CC2)n1]

5-[4-[(4,6-dimethylpyrimidin-2-yl)amino]cyclohexoxy]-7-morpholino-1,6-naphthyridin-3-ol (50.mg, 0.1100 mmol), 5-(1-chloroethyl)-1-methyl-2-nitro-imidazole (25.25 mg, 0.1300 mmol) and Potassium carbonate (53.69 mg, 0.3900 mmol) were transferred to a 10 mL RB flask and stirred at ambient temperature under an argon atmosphere. DMF (1 mL) was added and the mixture stirred at room temperature for 16 h. Sample filtered through a syringe filter, washed through with DMSO and combined filtrate and washings purified by basic prep HPLC (4×300 uL) injections to give a yellow solid. Triturated with IPA (1 mL) and left standing over the weekend and resultant solid filtered off and dried under vacuum to afford a yellow solid. Which was further dried in a vacuum oven at 40-C for 3 h to afford 4,6-dimethyl-N-[4-[[3-[1-(3-methyl-2-nitro-imidazol-4-yl)ethoxy]-7-morpholino-1,6-naphthyridin-5-yl]oxy]cyclohexyl]pyrimidin-2-amine (13.5 mg, 20% yield) as a yellow solid.

1H NMR (400 MHz, DMSO) δ: 8.69 (d, J=3.0 Hz, 1H), 7.77 (d, J=3.0 Hz, 1H), 7.38 (s, 1H), 6.83 (d, J=8.0 Hz, 1H), 6.52 (s, 1H), 6.32 (s, 1H), 5.97 (q, J=6.3 Hz, 1H), 5.42-5.34 (m, 1H), 3.99 (s, 3H), 3.96-3.85 (m, 1H), 3.79-3.68 (m, 4H), 3.49-3.39 (m, 4H), 2.19 (s, 6H), 2.12-1.93 (m, 2H), 1.88-1.66 (m, 9H).

LC-MS (long, acidic method) 2.08 min, 604.3 [M+H]+

### N-[4-[[3-[(3-methyl-2-nitro-imidazol-4-yl)methoxy]-7-morpholino-1,6-naphthyridin-5-yl]oxy]cyclohexyl]pyridin-2-amine (LFA2083)

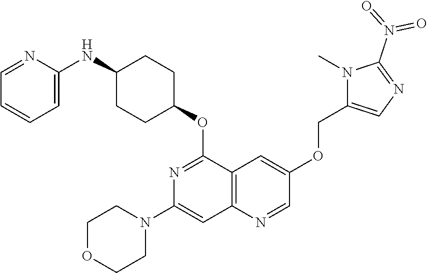 [SMILES: Cn1c(COc2cnc3cc(N4CCOCC4)nc(O[C@H]4CC[C@@H](Nc5ccccn5)CC4)c3c2)cnc1[N+](=O)[O-]]

A mixture of 7-morpholino-5-[4-(2-pyridylamino)cyclohexoxy]-1,6-naphthyridin-3-ol (48.mg, 0.1100 mmol), 5-(chloromethyl)-1-methyl-2-nitro-imidazole (23.99 mg, 0.1400 mmol) and Potassium carbonate (39.35 mg, 0.2800 mmol) in DMF (1 mL) was stirred at RT for 20 h. Water (10 mL) was added and the resultant precipitate was filtered, washed with water (2×4 mL) and dried by drawing air through the filter cake for 30 min. The collected solid was dissolved in MeOH (20 mL) and the solution was concentrated in vacuo to give the crude product as a brown/orange gum. The crude product was purified by flash column chromatography (10 g silica column, eluting with 0-5% 2 M NH3/MeOH in DCM) to give the product as an orange gum. The orange gum was further purified by basic prep. HPLC (method: basic prep.; 5-95% 0.005 M NH₄OH/MeCN in 0.005 M NH₄OH/H2O), then by basic prep. HPLC using a focused gradient (method: basic prep.; 10-39-49-95% 0.005 M NH₄OH/MeCN in 0.005 M NH₄OH/H2O) to give the product, N-[4-[[3-[(3-methyl-2-nitro-imidazol-4-yl)methoxy]-7-morpholino-1,6-naphthyridin-5-yl]oxy]cyclohexyl]pyridin-2-amine (21 mg, 33% yield), as a yellow solid.

1H NMR (400 MHz, DMSO-d6) δ: 8.69 (d, J=3.0 Hz, 1H), 7.95 (dd, J=5.2, 1.9 Hz, 1H), 7.83 (d, J=3.0 Hz, 1H), 7.40 (s, 1H), 7.33 (ddd, J=8.7, 6.9, 2.0 Hz, 1H), 6.54 (s, 1H), 6.50 (d, J=8.4 Hz, 1H), 6.48-6.32 (m, 2H), 5.44 (s, 2H), 5.42-5.34 (m, 1H), 4.00 (s, 3H), 3.95-3.85 (m, 1H), 3.82-3.65 (m, 4H), 3.52-3.40 (m, 4H), 2.17-2.00 (m, 2H), 1.91-1.63 (m, 6H). LCMS (long, acidic): 1.75 min, 561.2 [M+H]+

### N-[4-[[3-[1-(3-methyl-2-nitro-imidazol-4-yl)ethoxy]-7-morpholino-1,6-naphthyridin-5-yl]oxy]cyclohexyl]pyridin-2-amine (LFA2084)

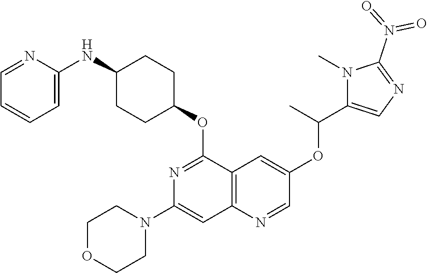 [SMILES: CC(Oc1cnc2cc(N3CCOCC3)nc(O[C@H]3CC[C@@H](Nc4ccccn4)CC3)c2c1)c1cnc([N+](=O)[O-])n1C]

A mixture of 7-morpholino-5-[4-(2-pyridylamino)cyclohexoxy]-1,6-naphthyridin-3-ol (42.mg, 0.1000 mmol), 5-(1-chloroethyl)-1-methyl-2-nitro-imidazole (22.67 mg, 0.1200 mmol) and Potassium carbonate (34.43 mg, 0.2500 mmol) in DMF (1 mL) was stirred at RT for 20 h. Water (5 mL) was added and the resultant precipitate was filtered, washed with water (2×4 mL) and dried by drawing air through the filter cake for 30 min. The collected solid was dissolved in MeOH (20 mL) and the solution was concentrated in vacuo to give the crude product as a yellow gum (52 mg). The yellow gum was purified by flash column chromatography (25 g silica column, eluting with 50-100% EtOAc in DCM) to give the product as a yellow gum (47 mg). The yellow gum was further purified by basic prep. HPLC (method: basic prep.; 5-95% 0.005 M NH₄OH/MeCN in 0.005 M NH₄OH/H2O) to give the product as a yellow gum. The gum was suspended in TBME (1 mL) and concentrated in vacuo to give the product, N-[4-[[3-[1-(3-methyl-2-nitro-imidazol-4-yl)ethoxy]-7-morpholino-1,6-naphthyridin-5-yl]oxy]cyclohexyl]pyridin-2-amine (33 mg, 58% yield), as a yellow solid.

1H NMR (400 MHz, DMSO-d6) δ: 8.70 (d, J=3.0 Hz, 1H), 7.95 (ddd, J=5.1, 1.9, 0.8 Hz, 1H), 7.75 (dd, J=3.0, 0.7 Hz, 1H), 7.38 (s, 1H), 7.37-7.31 (m, 1H), 6.54-6.48 (m, 2H), 6.43 (ddd, J=7.0, 5.0, 1.0 Hz, 1H), 6.37 (d, J=7.7 Hz, 1H), 5.97 (q, J=6.4 Hz, 1H), 5.40-5.32 (m, 1H), 3.99 (s, 3H), 3.95-3.82 (m, 1H), 3.73 (dd, J=5.9, 3.8 Hz, 4H), 3.43 (dd, J=6.3, 3.5 Hz, 4H), 2.13-1.98 (m, 2H), 1.88-1.76 (m, 4H), 1.74 (d, J=6.5 Hz, 3H), 1.71-1.56 (m, 2H).

LCMS (long, acidic): 1.78 min, 575.2 [M+H]+

### 5-[(dimethylamino)methyl]-N-[4-[[3-[1-(3-methyl-2-nitro-imidazol-4-yl)ethoxy]-7-morpholino-1,6-naphthyridin-5-yl]oxy]cyclohexyl]pyrimidin-2-amine (LFA2085)

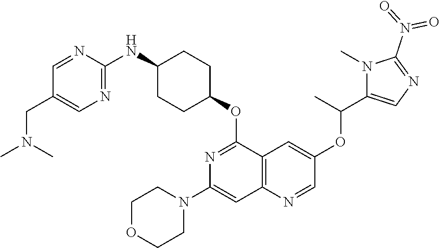 [SMILES: CC(Oc1cnc2cc(N3CCOCC3)nc(O[C@H]3CC[C@@H](Nc4ncc(CN(C)C)cn4)CC3)c2c1)c1cnc([N+](=O)[O-])n1C]

5-[4-[[5-[(dimethylamino)methyl]pyrimidin-2-yl]amino]cyclohexoxy]-7-morpholino-1,6-naphthyridin-3-ol (25.mg, 0.0500 mmol) and Potassium carbonate (14.41 mg, 0.1000 mmol) were transferred to a 10 mL RB flask and stirred at ambient temperature under an argon atmosphere. 5-(1-chloroethyl)-1-methyl-2-nitro-imidazole (10.87 mg, 0.0600 mmol) dissolved in DMF (0.5000 mL) was added and the mixture stirred at room temperature for 18 h. Sample filtered through a syringe filter, washed through with DMSO and combined filtrate and washings purified by basic prep HPLC (2×400 uL) injections. Relevant fractions were combined and concentrated to dryness in vacuo to afford 5-[(dimethylamino)methyl]-N-[4-[[3-[1-(3-methyl-2-nitro-imidazol-4-yl)ethoxy]-7-morpholino-1,6-naphthyridin-5-yl]oxy]cyclohexyl]pyrimidin-2-amine (14.1 mg, 43% yield) as a yellow solid.

1H NMR (400 MHz, DMSO) δ: 8.70 (d, J=3.0 Hz, 1H), 8.16 (s, 2H), 7.76 (dd, J=3.0, 0.8 Hz, 1H), 7.37 (s, 1H), 7.04 (d, J=7.9 Hz, 1H), 6.52 (d, J=0.8 Hz, 1H), 5.97 (q, J=6.5 Hz, 1H), 5.41-5.36 (m, 1H), 3.99 (s, 3H), 3.95-3.82 (m, 1H), 3.77-3.70 (m, 4H), 3.46-3.39 (m, 4H), 3.19 (s, 2H), 2.11 (s, 6H), 2.09-1.98 (m, 2H), 1.84-1.69 (m, 9H).

LC-MS (long, acidic method) rt 1.63 min rt 1.67 min, 633.3 [M+H]+

### 5-[(dimethylamino)methyl]-N-[4-[[3-[(3-methyl-2-nitro-imidazol-4-yl)methoxy]-7-morpholino-1,6-naphthyridin-5-yl]oxy]cyclohexyl]pyrimidin-2-amine (LFA2086)

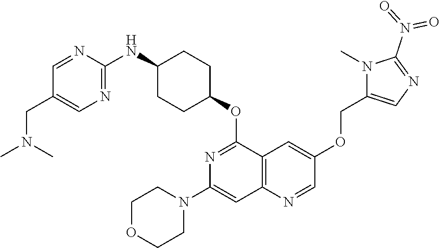 [SMILES: CN(C)Cc1cnc(N[C@H]2CC[C@@H](Oc3nc(N4CCOCC4)cc4ncc(OCc5cnc([N+](=O)[O-])n5C)cc34)CC2)nc1]

5-[4-[[5-[(dimethylamino)methyl]pyrimidin-2-yl]amino]cyclohexoxy]-7-morpholino-1,6-naphthyridin-3-ol (24.1 mg, 0.0500 mmol), 5-(chloromethyl)-1-methyl-2-nitro-imidazole (9.71 mg, 0.0600 mmol) and Potassium carbonate (13.89 mg, 0.1000 mmol) were transferred to a 10 mL RB flask and stirred at ambient temperature under an argon atmosphere. DMF (0.5000 mL) was added and the mixture stirred at room temperature for 6 h. Sample filtered through a syringe filter, washed through with DMSO and combined filtrate and washings purified by basic prep HPLC (3×300 uL) injections. No purification achieved and relevant fractions were combined and concentrated to dryness in vacuo. The residue was further purified by acidic prep HPLC (2×200 uL injections in DMSO). Free based by passing down 2 g SCX cartridges, washing with MeOH and eluting the product off with 2M ammonia in MeOH. Further purified by eluting onto a 5 g silica cartridge in DCM and eluting with 2M ammonia in MeOH (0-10%) in DCM. Relevant fractions were combined and concentrated to dryness in vacuo to afford 5-[(dimethylamino)methyl]-N-[4-[[3-[(3-methyl-2-nitro-imidazol-4-yl)methoxy]-7-morpholino-1,6-naphthyridin-5-yl]oxy]cyclohexyl]pyrimidin-2-amine (5.5 mg, 18% yield) as an orange solid.

1H NMR (400 MHz, DMSO) δ: 8.70 (d, J=3.0 Hz, 1H), 8.16 (s, 2H), 7.85 (d, J=3.0 Hz, 1H), 7.41 (s, 1H), 7.04 (d, J=7.8 Hz, 1H), 6.55 (s, 1H), 5.45 (s, 2H), 5.44-5.40 (m, 1H), 4.02 (s, 3H), 3.96-3.85 (m, 1H), 3.79-3.72 (m, 4H), 3.48-3.42 (m, 4H), 3.19 (s, 2H), 2.14-2.05 (m, 8H), 1.86-1.73 (m, 6H).

LC-MS (long, acidic method) rt 1.61 min and rt 1.65 min, 619.3 [M+H]+

### 5-[2-(dimethylamino)ethoxy]-N-[4-[[3-[1-(3-methyl-2-nitro-imidazol-4-yl)ethoxy]-7-morpholino-1,6-naphthyridin-5-yl]oxy]cyclohexyl]pyrimidin-2-amine (LFA2087)

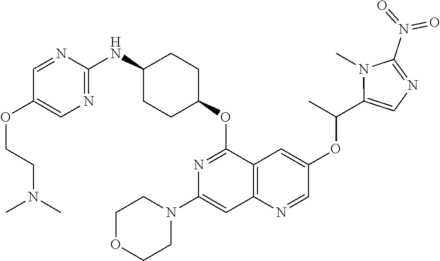 [SMILES: CC(Oc1cnc2cc(N3CCOCC3)nc(O[C@H]3CC[C@@H](Nc4ncc(OCCN(C)C)cn4)CC3)c2c1)c1cnc([N+](=O)[O-])n1C]

A mixture of 5-[4-[[5-[2-(dimethylamino)ethoxy]pyrimidin-2-yl]amino]cyclohexoxy]-7-morpholino-1,6-naphthyridin-3-ol (28.mg, 0.0500 mmol), 5-(1-chloroethyl)-1-methyl-2-nitro-imidazole (12.5 mg, 0.0700 mmol) and Potassium carbonate (18.98 mg, 0.1400 mmol) in DMF (1 mL) was stirred at RT for 18 h. A further portion of 5-(1-chloroethyl)-1-methyl-2-nitro-imidazole (4.mg, 0.0200 mmol) was added and the mixture was stirred at RT for a further 24 h. The mixture was diluted with water (10 mL) and brine (10 mL) and was extracted with EtOAc (3×20 mL). The combined organic phases were dried (Na2SO4), filtered and concentrated in vacuo to give the crude product as a brown gum. The brown gum was purified by flash column chromatography (12 g Redisep Gold silica column, eluting with 0-8% 2 M NH3/MeOH in DCM) to give the product as a yellow gum. The yellow gum was further purified by basic prep. HPLC (method: basic prep.; 5-95% 0.005 M NH₄OH/MeCN in 0.005 M NH₄OH/H2O) to give the product, 5-[2-(dimethylamino)ethoxy]-N-[4-[[3-[1-(3-methyl-2-nitro-imidazol-4-yl)ethoxy]-7-morpholino-1,6-naphthyridin-5-yl]oxy]cyclohexyl]pyrimidin-2-amine (13 mg, 36% yield), as a yellow solid.

1H NMR (400 MHz, DMSO-d6) δ: 8.69 (d, J=3.0 Hz, 1H), 8.12 (s, 2H), 7.76 (d, J=3.0 Hz, 1H), 7.37 (s, 1H), 6.69 (d, J=7.9 Hz, 1H), 6.51 (d, J=0.7 Hz, 1H), 5.97 (q, J=6.4 Hz, 1H), 5.41-5.35 (m, 1H), 4.01 (t, J=5.8 Hz, 2H), 3.99 (s, 3H), 3.84-3.76 (m, 1H), 3.76-3.70 (m, 4H), 3.48-3.39 (m, 4H), 2.56 (t, J=5.8 Hz, 2H), 2.19 (s, 6H), 2.13-1.97 (m, 2H), 1.87-1.67 (m, 9H).

LCMS (long, acidic): 1.70 min, 663.3 [M+H]+

### 5-[2-(dimethylamino)ethoxy]-N-[4-[[3-[(3-methyl-2-nitro-imidazol-4-yl)methoxy]-7-morpholino-1,6-naphthyridin-5-yl]oxy]cyclohexyl]pyrimidin-2-amine (LFA2088)

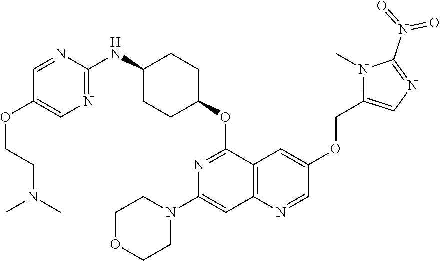 [SMILES: CN(C)CCOc1cnc(N[C@H]2CC[C@@H](Oc3nc(N4CCOCC4)cc4ncc(OCc5cnc([N+](=O)[O-])n5C)cc34)CC2)nc1]

A mixture of 5-[4-[[5-[2-(dimethylamino)ethoxy]pyrimidin-2-yl]amino]cyclohexoxy]-7-morpholino-1,6-naphthyridin-3-ol (38.mg, 0.0700 mmol), 5-(chloromethyl)-1-methyl-2-nitro-imidazole (14.4 mg, 0.0800 mmol) and Potassium carbonate (25.77 mg, 0.1900 mmol) in DMF (1 mL) was stirred at RT for 67 h. The mixture was diluted with EtOAc (20 mL) and washed with 1:1 water/brine (20 mL). The aqueous phase was extracted with EtOAc (2×20 mL) and the combined organic phases were dried (Na2SO4), filtered and concentrated in vacuo to give the crude product as an orange/brown gum. This was purified by flash column chromatography (12 g Redisep Gold silica column, eluting with 0-5% 2 M NH3/MeOH in DCM). Purified further by basic prep. HPLC (method: basic prep.; 5-95% 0.005 M NH₄OH/MeCN in 0.005 M NH₄OH/H2O) and the product-containing fractions combined and dried to give the product, 5-[2-(dimethylamino)ethoxy]-N-[4-[[3-[(3-methyl-2-nitro-imidazol-4-yl)methoxy]-7-morpholino-1,6-naphthyridin-5-yl]oxy]cyclohexyl]pyrimidin-2-amine (6 mg, 12% yield), as a yellow solid.

1H NMR (400 MHz, DMSO-d6) δ: 8.69 (d, J=3.0 Hz, 1H), 8.11 (s, 2H), 7.84 (d, J=3.0 Hz, 1H), 7.39 (s, 1H), 6.69 (d, J=7.9 Hz, 1H), 6.54 (s, 1H), 5.44 (s, 2H), 5.43-5.39 (m, 1H), 4.01 (s, 3H), 4.00 (t, J=5.8 Hz, 2H), 3.87-3.78 (m, 1H), 3.76-3.68 (m, 4H), 3.48-3.40 (m, 4H), 2.56 (t, J=5.8 Hz, 2H), 2.19 (s, 6H), 2.14-2.03 (m, 2H), 1.88-1.71 (m, 6H).

LCMS (long, acidic): 1.68 min, 649.3 [M+H]+

### N-[4-[[3-[1-(1-methyl-5-nitro-pyrazol-4-yl)ethoxy]-7-morpholino-1,6-naphthyridin-5-yl]oxy]cyclohexyl]-5-[2-(4-methylpiperazin-1-yl)ethoxy]pyrimidin-2-amine (LFA2089)

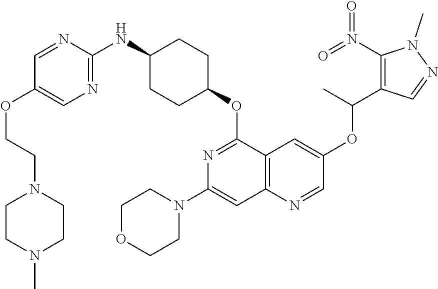 [SMILES: CC(Oc1cnc2cc(N3CCOCC3)nc(O[C@H]3CC[C@@H](Nc4ncc(OCCN5CCN(C)CC5)cn4)CC3)c2c1)c1cnn(C)c1[N+](=O)[O-]]

5-[4-[[5-[2-(4-methylpiperazin-1-yl)ethoxy]pyrimidin-2-yl]amino]cyclohexoxy]-7-morpholino-1,6-naphthyridin-3-ol (39.mg, 0.0700 mmol), 4-(1-chloroethyl)-1-methyl-5-nitro-pyrazole (15.71 mg, 0.0800 mmol) and Potassium carbonate (23.86 mg, 0.1700 mmol) in DMF (1 mL) were stirred at 100 C for 18 h, then allowed to stand at RT for 4 d. The reaction mixture was diluted with water (10 mL) and brine (10 mL) and extracted with 5% MeOH/EtOAc (3×20 mL). The combined organic phases were dried (phase-separator) and concentrated in vacuo to give the crude product as a brown gum. The crude product was purified by flash column chromatography (12 g Redisep Gold silica column, eluting with 0-10% 2 M NH₃/MeOH in DCM) to give a yellow gum. The yellow gum was purified by basic prep. HPLC (method: basic prep.; 5-95% 0.005 M NH₄OH/MeCN in 0.005 M NH₄OH/H2O) to give the product, N-[4-[[3-[1-(1-methyl-5-nitro-pyrazol-4-yl)ethoxy]-7-morpholino-1,6-naphthyridin-5-yl]oxy]cyclohexyl]-5-[2-(4-methylpiperazin-1-yl)ethoxy]pyrimidin-2-amine (4 mg, 8% yield), as a yellow solid.

1H NMR (400 MHz, DMSO-d6) δ: 8.63 (d, J=3.0 Hz, 1H), 8.12 (s, 2H), 7.83 (s, 1H), 7.62 (d, J=3.0 Hz, 1H), 6.55-6.42 (m, 2H), 5.97 (q, J=6.3 Hz, 1H), 5.37-5.25 (m, 1H), 4.11 (s, 3H), 4.04 (t, J=5.8 Hz, 2H), 3.84-3.75 (m, 1H), 3.77-3.68 (m, 4H), 3.45-3.37 (m, 4H), 2.62 (t, J=5.8 Hz, 2H), 2.56-2.41 (m, 8H), 2.32-2.22 (m, 2H), 2.14 (s, 3H), 2.04 (d, J=14.3 Hz, 2H), 1.85-1.58 (m, 4H), 1.70 (d, J=6.3 Hz, 3H).

LCMS (long, acidic): 1.78 min, 718.3 [M+H]+

### 5-methyl-N-[4-[[3-[1-(3-methyl-2-nitro-imidazol-4-yl)ethoxy]-7-morpholino-1,6-naphthyridin-5-yl]oxy]cyclohexyl]pyridin-2-amine (LFA2105)

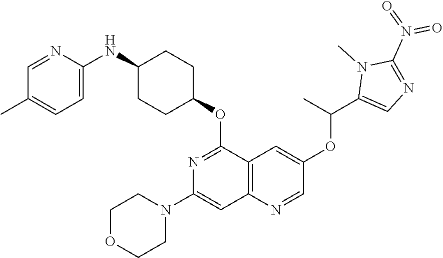 [SMILES: Cc1ccc(N[C@H]2CC[C@@H](Oc3nc(N4CCOCC4)cc4ncc(OC(C)c5cnc([N+](=O)[O-])n5C)cc34)CC2)nc1]

A mixture of 5-[4-[(5-methyl-2-pyridyl)amino]cyclohexoxy]-7-morpholino-1,6-naphthyridin-3-ol (58.mg, 0.1300 mmol), 5-(1-chloroethyl)-1-methyl-2-nitro-imidazole (27.77 mg, 0.1500 mmol) and Potassium carbonate (46.01 mg, 0.3300 mmol) in DMF (1 mL) was stirred at room temperature for 16 hours. The reaction mixture was filtered and purified by basic prep HPLC. The appropriate fractions were combined and the solvent removed at reduced pressure to afford 5-methyl-N-[4-[[3-[1-(3-methyl-2-nitro-imidazol-4-yl)ethoxy]-7-morpholino-1,6-naphthyridin-5-yl]oxy]cyclohexyl]pyridin-2-amine (27.8 mg, 35% yield).

1H NMR (400 MHz, DMSO) δ: 8.69 (d, J=3.0 Hz, 1H), 7.79 (d, J=2.5 Hz, 1H), 7.74 (d, J=3.0 Hz, 1H), 7.37 (s, 1H), 7.20 (dd, J=8.4, 2.4 Hz, 1H), 6.52 (s, 1H), 6.45 (d, J=8.5 Hz, 1H), 6.13 (d, J=7.8 Hz, 1H), 5.97 (q, J=6.4 Hz, 1H), 5.37-5.33 (m, 1H), 3.99 (s, 3H), 3.83 (d, J=9.0 Hz, 1H), 3.73 (t, J=4.8 Hz, 4H), 3.43 (t, J=4.9 Hz, 4H), 2.12-1.97 (m, 5H), 1.79 (d, J=11.4 Hz, 4H), 1.76-1.72 (m, 3H), 1.71-1.59 (m, 2H).

LCMS (long, acidic): 1.751 min, 589.2 [M+H]+

### N-[1-(3-methyl-2-nitro-imidazol-4-yl)ethyl]-5-[4-[[5-[2-(4-methylpiperazin-1-yl)ethoxy]pyrimidin-2-yl]amino]cyclohexoxy]-7-morpholino-1,6-naphthyridine-3-sulfonamide (LFA2090)

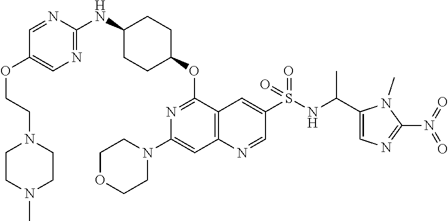 [SMILES: CC(NS(=O)(=O)c1cnc2cc(N3CCOCC3)nc(O[C@H]3CC[C@@H](Nc4ncc(OCCN5CCN(C)CC5)cn4)CC3)c2c1)c1cnc([N+](=O)[O-])n1C]

tert-butyl N-[1-(3-methyl-2-nitro-imidazol-4-yl)ethyl]-N-[[5-[4-[[5-[2-(4-methylpiperazin-1-yl)ethoxy]pyrimidin-2-yl]amino]cyclohexoxy]-7-morpholino-1,6-naphthyridin-3-yl]sulfonyl]carbamate (23.6 mg, 0.0200 mmol) was dissolved in DCM (700 uL) and stirred at RT while Trifluoroacetic acid (350.uL, 4.57 mmol) was added in one portion. The red solution was stirred at RT for 2 h and then concentrated to dryness under reduced pressure. The residue was loaded onto a 1 g SCX cartridge in MeOH, washed with MeOH and the product eluted off with 2M ammonia in MeOH. Relevant fractions were combined to give a yellow gum. Further purified by acidic prep HPLC (1×400 uL in DMSO) and relevant fractions were combined and free based by loading onto a 0.5 g SCX cartridge, washing with MeOH and eluting the product off with 2M ammonia in MeOH. Relevant fractions were combined and concentrated in vacuo to give a yellow solid. Further purified by basic prep HPLC (1×500 uL injection in DMSO). Relevant fractions were combined and concentrated in vacuo to give N-[1-(3-methyl-2-nitro-imidazol-4-yl)ethyl]-5-[4-[[5-[2-(4-methylpiperazin-1-yl)ethoxy]pyrimidin-2-yl]amino]cyclohexoxy]-7-morpholino-1,6-naphthyridine-3-sulfonamide (6.8 mg, 36% yield) as a yellow solid.

1H NMR (400 MHz, DMSO) δ:8.90 (d, J=2.5 Hz, 1H), 8.57 (s, 1H), 8.37 (dd, J=2.4, 0.8 Hz, 1H), 8.11 (s, 2H), 7.03 (s, 1H), 6.78 (d, J=7.6 Hz, 1H), 6.56 (s, 1H), 5.39-5.32 (m, 1H), 4.69 (q, J=7.0 Hz, 1H), 4.03 (t, J=5.8 Hz, 2H), 3.83-3.69 (m, 8H), 3.64-3.57 (m, 4H), 2.62 (t, J=5.8 Hz, 2H), 2.48-2.20 (m, 8H), 2.19-2.08 (m, 5H), 1.87-1.67 (m, 6H), 1.36 (d, J=6.9 Hz, 3H).

LC-MS (long, acidic method) rt 1.67 min, 781.3 [M+H]+

tert-butyl N-[1-(3-methyl-2-nitro-imidazol-4-yl)ethyl]-N-[[5-[4-[[5-[2-(4-methylpiperazin-1-yl)ethoxy]pyrimidin-2-yl]amino]cyclohexoxy]-7-morpholino-1,6-naphthyridin-3-yl]sulfonyl]carbamate

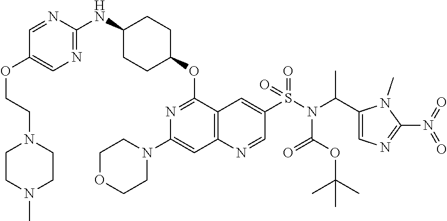 [SMILES: CC(c1cnc([N+](=O)[O-])n1C)N(C(=O)OC(C)(C)C)S(=O)(=O)c1cnc2cc(N3CCOCC3)nc(O[C@H]3CC[C@@H](Nc4ncc(OCCN5CCN(C)CC5)cn4)CC3)c2c1]

tert-butyl N-[[5-[4-[[5-[2-(4-methylpiperazin-1-yl)ethoxy]pyrimidin-2-yl]amino]cyclohexoxy]-7-morpholino-1,6-naphthyridin-3-yl]sulfonyl]carbamate (30.mg, 0.0400 mmol), 5-(1-chloroethyl)-1-methyl-2-nitro-imidazole (8.6 mg, 0.0500 mmol), Potassium carbonate (12.53 mg, 0.0900 mmol) and sodium iodide (6.18 mg, 0.0400 mmol) were transferred to a 10 mL RB flask and stirred at RT under an argon atmosphere. DMF (0.4000 mL) was added and the mixture stirred at RT for 7.5 h. Worked up by partitioning between water (20 mL) and EtOAc (3×20 mL). Combined extracts washed with brine (20 mL), dried over Na₂SO₄, filtered and concentrated in vacuo to give a yellow gum. Purified by loading onto a 5 g silica cartridge in DCM and eluting with 2M ammonia in MeOH (0-10%) in DCM. Relevant fractions were combined to give tert-butylN-[1-(3-methyl-2-nitro-imidazol-4-yl)ethyl]-N-[[5-[4-[[5-[2-(4-methylpiperazin-1-yl)ethoxy]pyrimidin-2-yl]amino]cyclohexoxy]-7-morpholino-1,6-naphthyridin-3-yl]sulfonyl]carbamate (23.6 mg, 58% yield) as a yellow solid.

1H NMR (400 MHz, CDCl3) δ: 8.94 (d, J=2.5 Hz, 1H), 8.54 (d, J=2.6 Hz, 1H), 8.03 (s, 2H), 7.26 (s, 1H), 7.23 (s, 2H), 6.49 (d, J=3.9 Hz, 1H), 5.77 (q, J=6.8 Hz, 1H), 5.37-5.32 (m, 1H), 5.00 (d, J=7.7 Hz, 1H), 4.02 (t, J=5.6 Hz, 2H), 3.89 (s, 3H), 3.84-3.77 (m, 4H), 3.68-3.58 (m, 4H), 2.74 (t, J=5.6 Hz, 2H), 2.69-2.31 (m, 8H), 2.26 (s, 3H), 2.18-2.05 (m, 2H), 1.99-1.90 (m, 5H), 1.90-1.80 (m, 2H), 1.74-1.69 (m, 2H), 1.34 (s, 9H).

LC-MS (long, acidic method) rt 1.94 min, 881.2 [M+H]+

### tert-butyl N-[[5-[4-[[5-[2-(4-methylpiperazin-1-yl)ethoxy]pyrimidin-2-yl]amino]cyclohexoxy]-7-morpholino-1,6-naphthyridin-3-yl]sulfonyl]carbamate

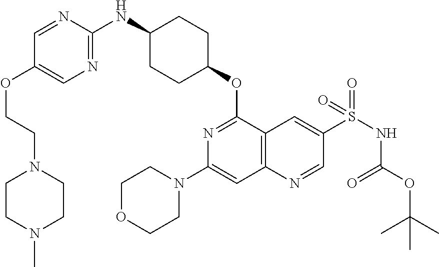 [SMILES: CN1CCN(CCOc2cnc(N[C@H]3CC[C@@H](Oc4nc(N5CCOCC5)cc5ncc(S(=O)(=O)NC(=O)OC(C)(C)C)cc45)CC3)nc2)CC1]

5-[4-[[5-[2-(4-methylpiperazin-1-yl)ethoxy]pyrimidin-2-yl]amino]cyclohexoxy]-7-morpholino-1,6-naphthyridine-3-sulfonamide (101.mg, 0.1600 mmol) was suspended in DCM (0.6 mL) and stirred at ambient temperature while Triethylamine (24.67 uL, 0.1800 mmol) and 4-dimethylaminopyridine (1.97 mg, 0.0200 mmol) was added in one portion. Di-tert-butyl dicarbonate (40.38 mg, 0.1900 mmol) dissolved in DCM (0.4 mL) was added dropwise over 10 min and the mixture stirred at RT for a further 2 h during which time dissolution had taken place affording a yellow solution. The mixture was partitioned between sat NaHCO₃ (10 mL) and DCM (3×10 mL) and the combined extracts dried (hydrophobic frit) and concentrated to dryness to give a yellow gum. Purified by loading onto a 5 g silica cartridge in DCM and eluting with 2M ammonia in MeOH (0-20%) in DCM. Relevant fractions were combined and concentrated to afford tert-butylN-[[5-[4-[[5-[2-(4-methylpiperazin-1-yl)ethoxy]pyrimidin-2-yl]amino]cyclohexoxy]-7-morpholino-1,6-naphthyridin-3-yl]sulfonyl]carbamate (99.3 mg, 85% yield) as a yellow gum.

1H NMR (400 MHz, DMSO) δ: 8.98 (d, J=2.3 Hz, 1H), 8.52 (dd, J=2.3, 0.8 Hz, 1H), 8.12 (s, 2H), 6.89 (d, J=7.6 Hz, 1H), 6.58 (d, J=0.9 Hz, 1H), 5.41-5.34 (m, 1H), 4.06 (t, J=5.5 Hz, 2H), 3.81-3.69 (m, 6H), 3.58-3.54 (m, 4H), 3.03-2.79 (m, 4H), 2.74 (t, J=5.5 Hz, 2H), 2.56 (s, 3H), 2.20-2.10 (m, 2H), 1.83-1.74 (m, 6H), 1.21 (s, 9H).

LC-MS (long, acidic method) rt 1.76 min, 728 [M+H]+, 32.7% @ 254 nm and rt 1.79 min, 728 [M+H], 60.8% @ 254 nm

### N-[(3-methyl-2-nitro-imidazol-4-yl)methyl]-7-morpholino-5-[4-[[5-(trifluoromethyl)pyrimidin-2-yl]amino]cyclohexoxy]-1,6-naphthyridine-3-sulfonamide (LFA2091)

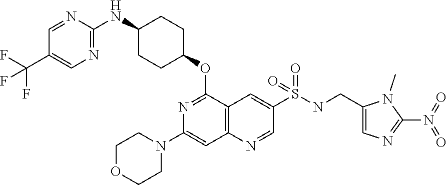 [SMILES: Cn1c(CNS(=O)(=O)c2cnc3cc(N4CCOCC4)nc(O[C@H]4CC[C@@H](Nc5ncc(C(F)(F)F)cn5)CC4)c3c2)cnc1[N+](=O)[O-]]

tert-butyl N-[(3-methyl-2-nitro-imidazol-4-yl)methyl]-N-[[7-morpholino-5-[4-[[5-(trifluoromethyl)pyrimidin-2-yl]amino]cyclohexoxy]-1,6-naphthyridin-3-yl]sulfonyl]carbamate (55.mg, 0.0700 mmol) was dissolved in DCM (2 mL) and stirred at RT while Trifluoroacetic acid (1.mL, 13.07 mmol) was added in one portion. The red solution was stirred at RT for 6 h. then concentrated to dryness under reduced pressure. The residue was loaded onto a 2 g SCX cartridge in MeOH, washed with MeOH and the product eluted off with 2 M ammonia in MeOH. Relevant fractions were combined to give a pale orange solid. Purified by basic prep HLC (3×300 uL injections in DMSO). Relevant fractions were combined and concentrated to afford a yellow solid. Further purified by acidic prep HPLC (2×300 uL in DMSO) and relevant fractions were combined and free based by loading onto a 2 g SCX cartridge, washing with MeOH and eluting the product off with 2M ammonia in MeOH. Relevant fractions were combined and concentrated in vacuo to give a yellow solid. Further dried in a vacuum oven at 40 C overnight to afford N-[(3-methyl-2-nitro-imidazol-4-yl)methyl]-7-morpholino-5-[4-[[5-(trifluoromethyl)pyrimidin-2-yl]amino]cyclohexoxy]-1,6-naphthyridine-3-sulfonamide (17.5 mg, 36% yield) as a yellow solid.

1H NMR (400 MHz, DMSO) δ: 8.96 (d, J=2.4 Hz, 1H), 8.65 (s, 1H), 8.60 (s, 1H), 8.44 (dd, J=2.5, 0.8 Hz, 1H), 8.37-8.10 (m, 2H), 6.99 (s, 1H), 6.60 (d, J=0.9 Hz, 1H), 5.41-5.35 (m, 1H), 4.26 (s, 2H), 4.02-3.89 (m, 1H), 3.76 (s, 3H), 3.75-3.71 (m, 4H), 3.66-3.56 (m, 4H), 2.24-2.09 (m, 2H), 1.88-1.72 (m, 6H).

LC-MS (long, acidic method) rt 2.67 min 693.2 [M+H]+

### tert-butyl N-[1-(3-methyl-2-nitro-imidazol-4-yl)ethyl]-N-[[7-morpholino-5-[4-[[5-(trifluoromethyl)pyrimidin-2-yl]amino]cyclohexoxy]-1,6-naphthyridin-3-yl]sulfonyl]carbamate

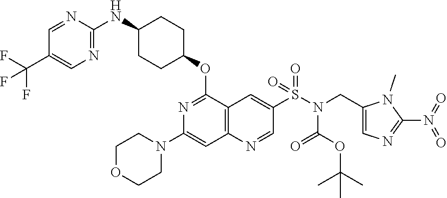 [SMILES: Cn1c(CN(C(=O)OC(C)(C)C)S(=O)(=O)c2cnc3cc(N4CCOCC4)nc(O[C@H]4CC[C@@H](Nc5ncc(C(F)(F)F)cn5)CC4)c3c2)cnc1[N+](=O)[O-]]

tert-butyl N-[[7-morpholino-5-[4-[[5-(trifluoromethyl)pyrimidin-2-yl]amino]cyclohexoxy]-1,6-naphthyridin-3-yl]sulfonyl]carbamate (40.mg, 0.0600 mmol), 5-(1-chloroethyl)-1-methyl-2-nitro-imidazole (12.76 mg, 0.0700 mmol), Potassium carbonate (18.61 mg, 0.1300 mmol) and sodium iodide (9.17 mg, 0.0600 mmol) were transferred to a 10 mL RB flask and stirred at RT under an argon atmosphere. DMF (0.5000 mL) was added and the mixture stirred at RT for 24 h. Worked up by partitioning between water (20 mL) and EtOAc (3×20 mL). Combined extracts washed with brine (20 mL), dried over Na2SO4, filtered and concentrated in vacuo to give a yellow gum. Purified by loading onto a 10 g silica cartridge in DCM and eluting with MeOH (0-5%) in DCM. Relevant fractions were combined to give tert-butylN-[1-(3-methyl-2-nitro-imidazol-4-yl)ethyl]-N-[[7-morpholino-5-[4-[[5-(trifluoromethyl)pyrimidin-2-yl]amino]cyclohexoxy]-1,6-naphthyridin-3-yl]sulfonyl]carbamate (43.6 mg, 88% yield) as a yellow solid.

1H NMR (400 MHz, DMSO) δ: 8.79 (d, J=2.5 Hz, 1H), 8.64 (s, 1H), 8.60 (s, 1H), 8.44 (d, J=2.5 Hz, 1H), 8.17 (d, J=7.5 Hz, 1H), 7.34 (d, J=0.8 Hz, 1H), 6.64 (s, 1H), 5.84 (q, J=6.8 Hz, 1H), 5.40-5.34 (m, 1H), 3.99-3.88 (m, 1H), 3.76-3.70 (m, 4H), 3.70-3.63 (m, 7H), 2.23-2.06 (m, 2H), 1.88 (d, 3H), 1.85-1.69 (m, 6H), 1.34 (s, 9H).

LC-MS (long, acidic method) rt 3.12 min 807.2 [M+H]+

### tert-butyl N-[[7-morpholino-5-[4-[[5-(trifluoromethyl)pyrimidin-2-yl]amino]cyclohexoxy]-1,6-naphthyridin-3-yl]sulfonyl]carbamate

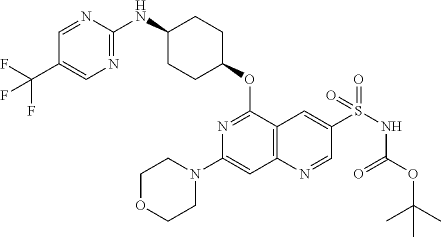 [SMILES: CC(C)(C)OC(=O)NS(=O)(=O)c1cnc2cc(N3CCOCC3)nc(O[C@H]3CC[C@@H](Nc4ncc(C(F)(F)F)cn4)CC3)c2c1]

7-morpholino-5-[4-[[5-(trifluoromethyl)pyrimidin-2-yl]amino]cyclohexoxy]-1,6-naphthyridine-3-sulfonamide (175.5 mg, 0.3200 mmol) was suspended in DCM (1.2 mL) and stirred at ambient temperature while Triethylamine (48.61 uL, 0.3500 mmol) and 4-dimethylaminopyridine (3.87 mg, 0.0300 mmol) was added in one portion. Di-tert-butyl dicarbonate (79.57 mg, 0.3600 mmol) dissolved in DCM (0.8 mL) was added dropwise over 5 min and the mixture stirred at RT for a further 17 h (overnight) during which time dissolution had taken place affording a yellow solution. The mixture was partitioned between sat NaHCO₃ (20 mL) and DCM (3×20 mL) and the combined extracts dried (hydrophobic frit) and concentrated to dryness to give a yellow gum. Purified by loading onto a 10 g silica cartridge in DCM and eluting wit MeOH (0-10%) in DCM. Relevant fractions were combined and concentrated to afford tert-butylN-[[7-morpholino-5-[4-[[5-(trifluoromethyl)pyrimidin-2-yl]amino]cyclohexoxy]-1,6-naphthyridin-3-yl]sulfonyl]carbamate (214.7 mg, 104% yield) as a yellow gum.

1H NMR (400 MHz, DMSO) δ: 11.76 (s, 1H), 8.97 (d, J=2.4 Hz, 1H), 8.62 (d, J=2.4 Hz, 2H), 8.58 (s, 1H), 8.17 (d, J=7.5 Hz, 1H), 6.63 (d, J=0.9 Hz, 1H), 5.41-5.35 (m, 1H), 4.00-3.87 (m, 1H), 3.74-3.68 (m, 4H), 3.67-3.58 (m, 4H), 2.19-2.07 (m, 2H), 1.87-1.71 (m, 6H), 1.27 (s, 9H).

### N-[1-(3-methyl-2-nitro-imidazol-4-yl)ethyl]-7-morpholino-5-[4-[[5-(trifluoromethyl)pyrimidin-2-yl]amino]cyclohexoxy]-1,6-naphthyridine-3-sulfonamide (LFA2092)

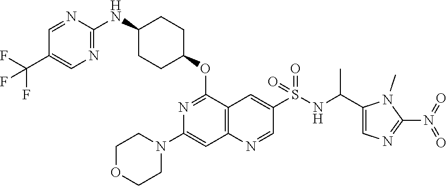 [SMILES: CC(NS(=O)(=O)c1cnc2cc(N3CCOCC3)nc(O[C@H]3CC[C@@H](Nc4ncc(C(F)(F)F)cn4)CC3)c2c1)c1cnc([N+](=O)[O-])n1C]

tert-butyl N-[1-(3-methyl-2-nitro-imidazol-4-yl)ethyl]-N-[[7-morpholino-5-[4-[[5-(trifluoromethyl)pyrimidin-2-yl]amino]cyclohexoxy]-1,6-naphthyridin-3-yl]sulfonyl]carbamate (42.2 mg, 0.0500 mmol) was dissolved in DCM (1.5 mL) and stirred at RT while Trifluoroacetic acid (0.75 mL, 9.85 mmol) was added in one portion. The red solution was stirred at RT for 6 h. Reaction mixture concentrated to dryness under reduced pressure and the residue loaded onto a 2 g SCX cartridge in MeOH, washed with MeOH and the product eluted off with 2 M ammonia in MeOH. Relevant fractions were combined to give a yellow solid. Further purified by acidic prep HPLC (2×300 uL in DMSO) and relevant fractions were combined and free based by loading onto a 2 g SCX cartridge, washing with MeOH and eluting the product off with 2M ammonia in MeOH. Relevant fractions were combined an concentrated in vacuo to give N-[1-(3-methyl-2-nitro-imidazol-4-yl)ethyl]-7-morpholino-5-[4-[[5-(trifluoromethyl)pyrimidin-2-yl]amino]cyclohexoxy]-1,6-naphthyridine-3-sulfonamidea yellow solid. Further dried in a vacuum oven at 40 C overnight to afford (19.2 mg, 52% yield) as a yellow solid.

1H NMR (400 MHz, DMSO) δ: 8.91 (d, J=2.4 Hz, 1H), 8.65 (s, 1H), 8.60 (s, 1H), 8.44 (s, 1H), 8.37 (dd, J=2.5, 0.8 Hz, 1H), 8.18 (d, J=7.6 Hz, 1H), 7.04 (s, 1H), 6.57 (d, J=0.9 Hz, 1H), 5.40-5.34 (m, 1H), 4.70 (q, J=6.9 Hz, 1H), 4.02-3.90 (m, 1H), 3.76 (s, 3H), 3.75-3.70 (m, 4H), 3.64-3.57 (m, 4H), 2.23-2.10 (m, 2H), 1.89-1.73 (m, 6H), 1.37 (d, J=6.9 Hz, 3H).

LC-MS (long, acidic method) rt 2.72 min 707.2 [M+H]+

### tert-butyl N-[1-(3-methyl-2-nitro-imidazol-4-yl)ethyl]-N-[[7-morpholino-5-[4-[[5-(trifluoromethyl)pyrimidin-2-yl]amino]cyclohexoxy]-1,6-naphthyridin-3-yl]sulfonyl]carbamate

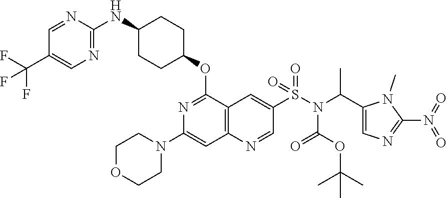 [SMILES: CC(c1cnc([N+](=O)[O-])n1C)N(C(=O)OC(C)(C)C)S(=O)(=O)c1cnc2cc(N3CCOCC3)nc(O[C@H]3CC[C@@H](Nc4ncc(C(F)(F)F)cn4)CC3)c2c1]

tert-butyl N-[[7-morpholino-5-[4-[[5-(trifluoromethyl)pyrimidin-2-yl]amino]cyclohexoxy]-1,6-naphthyridin-3-yl]sulfonyl]carbamate (40.mg, 0.0600 mmol), 5-(1-chloroethyl)-1-methyl-2-nitro-imidazole (12.76 mg, 0.0700 mmol), Potassium carbonate (18.61 mg, 0.1300 mmol) and sodium iodide (9.17 mg, 0.0600 mmol) were transferred to a 10 mL RB flask and stirred at RT under an argon atmosphere. DMF (0.5000 mL) was added and the mixture stirred at RT for 24 h. Worked up by partitioning between water (20 mL) and EtOAc (3×20 mL). Combined extracts washed with brine (20 mL), dried over Na2SO4, filtered and concentrated in vacuo to give a yellow gum. Purified by loading onto a 10 g silica cartridge in DCM and eluting with MeOH (0-5%) in DCM. Relevant fractions were combined to give tert-butylN-[1-(3-methyl-2-nitro-imidazol-4-yl)ethyl]-N-[[7-morpholino-5-[4-[[5-(trifluoromethyl)pyrimidin-2-yl]amino]cyclohexoxy]-1,6-naphthyridin-3-yl]sulfonyl]carbamate (43.6 mg, 88% yield) as a yellow solid.

1H NMR (400 MHz, DMSO) δ: 8.79 (d, J=2.5 Hz, 1H), 8.64 (s, 1H), 8.60 (s, 1H), 8.44 (d, J=2.5 Hz, 1H), 8.17 (d, J=7.5 Hz, 1H), 7.34 (d, J=0.8 Hz, 1H), 6.64 (s, 1H), 5.84 (q, J=6.8 Hz, 1H), 5.40-5.34 (m, 1H), 3.99-3.88 (m, 1H), 3.76-3.70 (m, 4H), 3.70-3.63 (m, 7H), 2.23-2.06 (m, 2H), 1.88 (d, 3H), 1.85-1.69 (m, 6H), 1.34 (s, 9H).

LC-MS (long, acidic method) rt 3.12 min 807.2 [M+H]+

### 6-methyl-2-[[4-[[3-[(3-methyl-2-nitro-imidazol-4-yl)methoxy]-7-morpholino-1,6-naphthyridin-5-yl]oxy]cyclohexyl]amino]pyrimidine-4-carbonitrile (LFA2093)

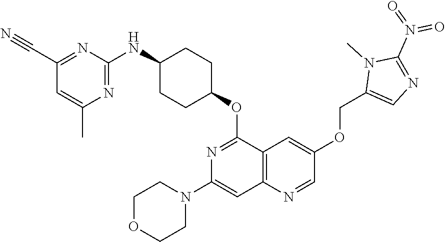 [SMILES: Cc1cc(C#N)nc(N[C@H]2CC[C@@H](Oc3nc(N4CCOCC4)cc4ncc(OCc5cnc([N+](=O)[O-])n5C)cc34)CC2)n1]

4-[[3-[(3-methyl-2-nitro-imidazol-4-yl)methoxy]-7-morpholino-1,6-naphthyridin-5-yl]oxy]cyclohexanamine (48.mg, 0.1000 mmol) and 2-chloro-6-methyl-pyrimidine-4-carbonitrile (22.87 mg, 0.1500 mmol) were transferred to a 10 mL microwave vial, suspended in Ethanol (1 mL) and Triethylamine (27.67 uL, 0.2000 mmol) added. The mixture was stirred at room temperature overnight (16 h) and then heated at 50 C for 4.5 h. Once cooled the mixture was concentrated to dryness under reduced pressure and the residue partitioned between water (20 mL) and DCM (2×20 mL). The combined organics were dried (hydrophobic frit) and concentrated to dryness under reduced pressure. The residue was purified by loading onto a 5 g silica column in DCM and eluting with MeOH (0-5%) in DCM. Relevant fractions were combined and concentrated to dryness in-vacuo to afford crude product as an orange solid. Further purified by basic prep HPLC (2×300 uL injections in DMSO) and the relevant fractions were combined and concentrated in-vacuo to afford 6-methyl-2-[[4-[[3-[(3-methyl-2-nitro-imidazol-4-yl)methoxy]-7-morpholino-1,6-naphthyridin-5-yl]oxy]cyclohexyl]amino]pyrimidine-4-carbonitrile (, 14.3 mg, 24% yield) as a yellow solid.

1H NMR (400 MHz, DMSO) δ: 8.69 (d, J=3.0 Hz, 1H), 7.83 (dd, J=3.0, 0.8 Hz, 1H), 7.76 (d, J=7.9 Hz, 1H), 7.40 (s, 1H), 7.01 (s, 1H), 6.55 (d, J=0.8 Hz, 1H), 5.44 (s, 2H), 5.43-5.38 (m, 1H), 4.01 (s, 3H), 3.99-3.80 (m, 1H), 3.79-3.70 (m, 4H), 3.48-3.41 (m, 4H), 2.31 (s, 3H), 2.13-2.03 (m, 2H), 1.86-1.73 (m, 6H).

LCMS (long, acidic): 2.71 min, 601.3 [M+H]+

### 4-[[3-[(3-methyl-2-nitro-imidazol-4-yl)methoxy]-7-morpholino-1,6-naphthyridin-5-yl]oxy]cyclohexanamine

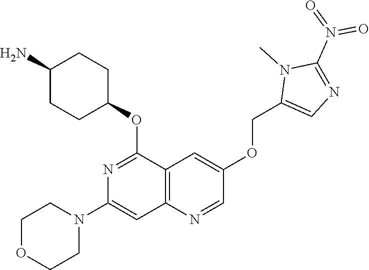 [SMILES: Cn1c(COc2cnc3cc(N4CCOCC4)nc(O[C@H]4CC[C@@H](N)CC4)c3c2)cnc1[N+](=O)[O-]]

tert-butyl N-[4-[[3-[(3-methyl-2-nitro-imidazol-4-yl)methoxy]-7-morpholino-1,6-naphthyridin-5-yl]oxy]cyclohexyl]carbamate (115.mg, 0.2000 mmol) was dissolved in DCM (1 mL) and stirred at room temperature while TFA (452.65 uL, 5.91 mmol) was added. The red solution was stirred at room temperature for 16 h and then the mixture concentrated to dryness under reduced pressure. The residue was dissolved in MeOH, loaded onto a 5 g SCX column and washed with MeOH. The product was eluted off the column in 2M ammonia in MeOH and relevant fractions were combined and concentrated in vacuo to afford 4-[[3-[(3-methyl-2-nitro-imidazol-4-yl)methoxy]-7-morpholino-1,6-naphthyridin-5-yl]oxy]cyclohexanamine (96 mg, 100% yield).

1H NMR (400 MHz, CDCl3)—8.61 (d, J=3.1 Hz, 1H), 7.75 (dd, J=3.1, 0.8 Hz, 1H), 7.31 (s, 1H), 6.54 (d, J=0.9 Hz, 1H), 5.44-5.38 (m, 1H), 5.20 (s, 2H), 4.12 (s, 3H), 3.90-3.83 (m, 4H), 3.53-3.46 (m, 4H), 2.99-2.88 (m, 1H), 2.23-2.15 (m, 2H), 1.80-1.56 (m, 8H).

LC-MS (long, acidic method) rt 1.607 min, 484.2 [M+H]+

### tert-butyl N-[4-[[3-[(3-methyl-2-nitro-imidazol-4-yl)methoxy]-7-morpholino-1,6-naphthyridin-5-yl]oxy]cyclohexyl]carbamate

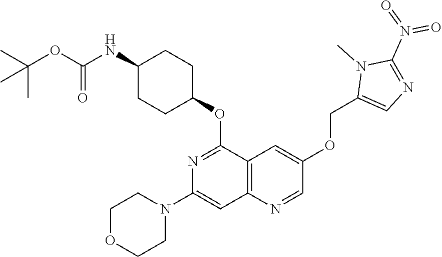 [SMILES: Cn1c(COc2cnc3cc(N4CCOCC4)nc(O[C@H]4CC[C@@H](NC(=O)OC(C)(C)C)CC4)c3c2)cnc1[N+](=O)[O-]]

tert-butyl N-[4-[(3-hydroxy-7-morpholino-1,6-naphthyridin-5-yl)oxy]cyclohexyl]carbamate (100.mg, 0.2200 mmol), 5-(chloromethyl)-1-methyl-2-nitro-imidazole (47.4 mg, 0.2700 mmol) and Potassium carbonate (77.73 mg, 0.5600 mmol) were combined in DMF (1 mL) and stirred at room temperature for 16 hours. The reaction was partitioned between water and EtOAc (×3) and combined extracts washed with brine, dried (Na2SO4), filtered and concentrated to dryness under reduced pressure. The resulting residue was purified by flash chromatography (10 g SiO2) eluting with a gradient 0-5% methanol in DCM. The appropriate fractions were combined and the solvent was removed at reduced pressure to afford tert-butyl N-[4-[[3-[(3-methyl-2-nitro-imidazol-4-yl)methoxy]-7-morpholino-1,6-naphthyridin-5-yl]oxy]cyclohexyl]carbamate (115 mg, 88% yield).

1H NMR (400 MHz, CDCl3) - 8.62 (d, J=3.1 Hz, 1H), 7.71 (dd, J=3.1, 0.8 Hz, 1H), 7.31 (s, 1H), 6.54 (d, J=0.9 Hz, 1H), 5.48-5.34 (m, 1H), 5.21 (s, 2H), 4.86 (s, 1H), 4.12 (s, 3H), 3.89-3.82 (m, 4H), 3.63-3.59 (m, 1H), 3.57-3.46 (m, 4H), 2.16-2.07 (m, 2H), 1.92-1.74 (m, 4H), 1.73-1.64 (m, 2H), 1.46 (s, 9H).

LC-MS (long, acidic method) rt 2.617 min, 584.2 [M+H]+

### tert-butyl N-[4-[(3-hydroxy-7-morpholino-1,6-naphthyridin-5-yl)oxy]cyclohexyl]carbamate

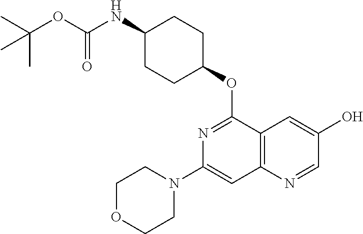 [SMILES: CC(C)(C)OC(=O)N[C@H]1CC[C@@H](Oc2nc(N3CCOCC3)cc3ncc(O)cc23)CC1]

Potassium hydroxide (7.91 M aq.) (4.1 mL, 32.47 mmol) was added to a mixture of tert-butyl N-[4-[(3-iodo-7-morpholino-1,6-naphthyridin-5-yl)oxy]cyclohexyl]carbamate (3000.mg, 5.41 mmol), tBuBrettPhos-Pd-G3 (231.17 mg, 0.2700 mmol) and tBuBrettPhos (131.13 mg, 0.2700 mmol) in 1,4-Dioxane (36.074 mL). The mixture was sparged with argon for 5 min. The mixture was heated to 90-C and stirred for 5 h. Acetic acid (3.1 mL, 54.11 mmol) was added and the mixture was stirred at room temperature for 10 min. The mixture was filtered through a pad of Celite, eluting with EtOAc. The filtrate was concentrated in vacuo to give the crude product as a yellow/orange solid. The crude product was purified by flash column chromatography (120 g silica column, eluting with 0-5% MeOH in DCM) to afford tert-butyl N-[4-[(3-hydroxy-7-morpholino-1,6-naphthyridin-5-yl)oxy]cyclohexyl]carbamate (2.203 g, 92% yield).

1H NMR (400 MHz, CDCl3)—8.71 (d, J=3.0 Hz, 1H), 7.81 (s, 1H), 6.56 (s, 1H), 5.42 (s, 1H), 4.68 (s, 1H), 3.90-3.83 (m, 4H), 3.58 (s, 1H), 3.51 (d, J=3.5 Hz, 4H), 2.16-2.09 (m, 2H), 2.00-1.81 (m, 6H), 1.48 (s, 9H).

LCMS (long, acidic): 2.130 min, 445.2 [M+H]+

### 6-methyl-2-[[4-[[3-[1-(3-methyl-2-nitro-imidazol-4-yl)ethoxy]-7-morpholino-1,6-naphthyridin-5-yl]oxy]cyclohexyl]amino]pyrimidine-4-carbonitrile (LFA2094)

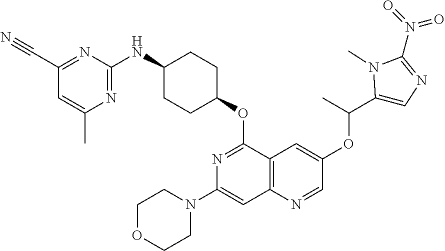 [SMILES: Cc1cc(C#N)nc(N[C@H]2CC[C@@H](Oc3nc(N4CCOCC4)cc4ncc(OC(C)c5cnc([N+](=O)[O-])n5C)cc34)CC2)n1]

Made in an analogous manner to LFA2093, using 5-(1-chloroethyl)-1-methyl-2-nitro-imidazole 4-[[3-[1-(3-methyl-2-nitro-imidazol-4-yl)ethoxy]-7-morpholino-1,6-naphthyridin-5-yl]oxy]cyclohexanamine (43.mg, 0.0900 mmol) and 2-chloro-6-methyl-pyrimidine-4-carbonitrile (19.91 mg, 0.1300 mmol) were transferred to a 10 mL microwave vial, suspended in Ethanol (1 mL) and Triethylamine (24.09 uL, 0.1700 mmol) added. The mixture was heated at 50 C for 2 h and at 75 C for 2 h. Once cooled the mixture was concentrated to dryness under reduced pressure and the residue partitioned between water (20 mL) and DCM (2×20 mL). The combined organics were dried (hydrophobic frit) and concentrated to dryness under reduced pressure. The residue was purified by loading onto a 5 g silica column in DCM and eluting with MeOH (0-3%) in DCM. Relevant fractions were combined and concentrated to dryness in-vacuo to afford crude product as an orange solid. Further purified by basic prep HPLC (3×300 uL injections in DMSO) and the relevant fractions were combined and concentrated in-vacuo to give 6-methyl-2-[[4-[[3-[1-(3-methyl-2-nitro-imidazol-4-yl)ethoxy]-7-morpholino-1,6-naphthyridin-5-yl]oxy]cyclohexyl]amino]pyrimidine-4-carbonitrile (20.9 mg, 38% yield)

1H NMR (400 MHz, DMSO) δ: 8.70 (d, J=3.0 Hz, 1H), 7.75 (d, J=3.3 Hz, 2H), 7.38 (s, 1H), 7.01 (s, 1H), 6.52 (d, J=0.9 Hz, 1H), 5.95 (q, J=6.4 Hz, 1H), 5.40-5.36 (m, 1H), 3.99 (s, 3H), 3.86 (s, 1H), 3.73 (t, J=4.8 Hz, 4H), 3.43 (t, J=4.9 Hz, 4H), 2.32 (s, 3H), 2.10-2.01 (m, 2H), 1.83-1.71 (m, 9H).

LC-MS (long, acidic method) rt 2.737 min, 615.2 [M+H]+

### 3-[[4-[[3-[(3-methyl-2-nitro-imidazol-4-yl)methoxy]-7-morpholino-1,6-naphthyridin-5-yl]oxy]cyclohexyl]amino]pyrazine-2-carbonitrile (LFA2095)

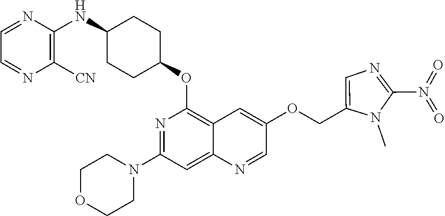 [SMILES: Cn1c(COc2cnc3cc(N4CCOCC4)nc(O[C@H]4CC[C@@H](Nc5nccnc5C#N)CC4)c3c2)cnc1[N+](=O)[O-]]

Prepared using standard conditions for alkylation of a phenol to give product (13 mg, 20%).

1H NMR (500 MHz, DMSO-d6) δ 8.72 (d, J=3.44 Hz, 1H), 8.36 (d, J=2.29 Hz, 1H), 7.91 (d, J=2.29 Hz, 1H), 7.86 (d, J=2.86 Hz, 1H), 7.37-7.40 (m, 2H), 6.55 (s, 1H), 5.46 (s, 2H), 5.40 (br. s., 1H), 4.05-4.13 (m, 1H), 4.00 (s, 3H), 3.72-3.76 (m, 4H), 3.42-3.46 (m, 4H), 2.09-2.16 (m, 2H), 1.90-1.96 (m, 2H), 1.76-1.82 (m, 4H).

LCMS: Rt=2.29 min., m/z at 587 MH+(MeOH, pH10).

### N-[4-[[3-[1-(3-methyl-2-nitro-imidazol-4-yl)ethoxy]-7-morpholino-1,6-naphthyridin-5-yl]oxy]cyclohexyl]-6-(trifluoromethyl)pyrazin-2-amine (LFA2096)

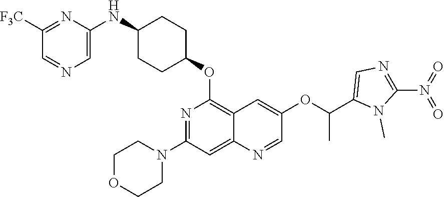 [SMILES: CC(Oc1cnc2cc(N3CCOCC3)nc(O[C@H]3CC[C@@H](Nc4cncc(C(F)(F)F)n4)CC3)c2c1)c1cnc([N+](=O)[O-])n1C]

Prepared using standard conditions for alkylation of a phenol to give product (10 mg, 28%).

1H NMR (500 MHz, DMSO-d6) δ 8.67-8.72 (m, 1H), 8.21-8.27 (m, 1H), 8.02-8.09 (m, 1H), 7.70-7.75 (m, 2H), 7.35-7.39 (m, 1H), 6.51-6.54 (m, 1H), 5.92-5.99 (m, 1H), 5.29-5.35 (m, 1H), 3.96-4.01 (m, 3H), 3.84-3.92 (m, 1H), 3.70-3.76 (m, 4H), 3.40-3.47 (m, 4H), 2.02-2.11 (m, 2H), 1.83-1.91 (m, 3H), 1.72-1.76 (m, 3H).

LCMS: Rt=1.80 min., m/z at 644 MH+(MeCN, pH1).

### N-[4-[[3-[(3-methyl-2-nitro-imidazol-4-yl)methoxy]-7-morpholino-1,6-naphthyridin-5-yl]oxy]cyclohexyl]-6-(trifluoromethyl)pyrazin-2-amine (LFA2097)

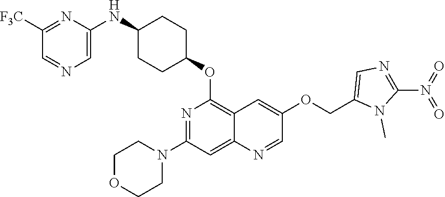 [SMILES: Cn1c(COc2cnc3cc(N4CCOCC4)nc(O[C@H]4CC[C@@H](Nc5cncc(C(F)(F)F)n5)CC4)c3c2)cnc1[N+](=O)[O-]]

Prepared using standard conditions for alkylation of a phenol to give product (7 mg, 12%).

1H NMR (500 MHz, DMSO-d6) δ 8.70 (d, J=2.86 Hz, 1H), 8.25 (s, 1H), 8.07 (s, 1H), 7.82 (d, J=2.86 Hz, 1H), 7.74 (d, J=6.87 Hz, 1H), 7.40 (s, 1H), 6.56 (s, 1H), 5.43 (s, 2H), 5.33-5.38 (m, 1H), 4.00 (s, 3H), 3.85-3.92 (m, 1H), 3.73-3.77 (m, 4H), 3.41-3.48 (m, 4H), 2.06-2.13 (m, 2H), 1.78-1.92 (m, 6H).

LCMS: Rt=1.71 min., m/z at 630 MH+(MeCN, pH1).

### N-[5-[4-[(4,6-dimethylpyrimidin-2-yl)amino]cyclohexoxy]-7-morpholino-1,6-naphthyridin-3-yl]-N-[(3-methyl-2-nitro-imidazol-4-yl)methyl]methanesulfonamide (LFA2098)

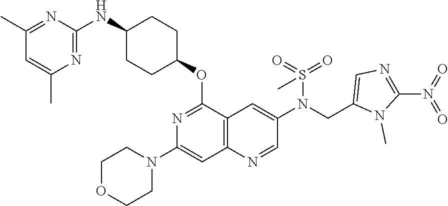 [SMILES: Cc1cc(C)nc(N[C@H]2CC[C@@H](Oc3nc(N4CCOCC4)cc4ncc(N(Cc5cnc([N+](=O)[O-])n5C)S(C)(=O)=O)cc34)CC2)n1]

Prepared using standard conditions for alkylation of a phenol to give product (13 mg, 1%).

1H NMR (500 MHz, METHANOL-d4) δ 8.65 (d, J=2.29 Hz, 1H), 8.32-8.34 (m, 1H), 6.92-6.94 (m, 1H), 6.47 (s, 1H), 6.40-6.42 (m, 1H), 5.43-5.47 (m, 1H), 5.19 (s, 2H), 4.09-4.12 (m, 3H), 4.00-4.07 (m, 1H), 3.79-3.83 (m, 4H), 3.58-3.61 (m, 4H), 3.17 (s, 3H), 2.28 (s, 6H), 2.14-2.20 (m, 2H), 1.88-1.97 (m, 4H), 1.78-1.85 (m, 2H).

LCMS. Rt=1.22 min., m/z at 667 MH+(MeCN, pH1).

### N-[1-(3-methyl-2-nitro-imidazol-4-yl)ethyl]-N-[7-morpholino-5-[4-[[6-(trifluoromethyl)pyrazin-2-yl]amino]cyclohexoxy]-1,6-naphthyridin-3-yl]methanesulfonamide (LFA2099)

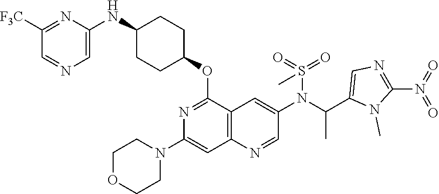 [SMILES: CC(c1cnc([N+](=O)[O-])n1C)N(c1cnc2cc(N3CCOCC3)nc(O[C@H]3CC[C@@H](Nc4cncc(C(F)(F)F)n4)CC3)c2c1)S(C)(=O)=O]

Prepared using standard conditions for alkylation of a phenol to give product

1H NMR (500 MHz, DMSO-d6) δ 8.39 (br. s., 1H), 8.25 (s, 1H), 8.07 (s, 1H), 7.77 (d, J=6.87 Hz, 1H), 7.68-7.72 (m, 1H), 6.82 (s, 1H), 6.53 (s, 1H), 5.70-5.76 (m, 1H), 5.27-5.31 (m, 1H), 4.16 (d, J=1.72 Hz, 3H), 3.86-3.91 (m, 1H), 3.71-3.75 (m, 4H), 3.73 (d, J=4.01 Hz, 4H), 3.51-3.55 (m, 4H), 3.53 (br. s., 4H), 3.30 (s, 3H), 2.03-2.09 (m, 2H), 1.91-2.00 (m, 2H), 1.78-1.89 (m, 6H), 1.63 (d, J=6.87 Hz, 4H).

LCMS: Rt=1.77 min., no mass ion (MeCN, pH1)

### N-[5-[4-[(3-cyanopyrazin-2-yl)amino]cyclohexoxy]-7-morpholino-1,6-naphthyridin-3-yl]-N-[1-(3-methyl-2-nitro-imidazol-4-yl)ethyl]methanesulfonamide (LFA2100)

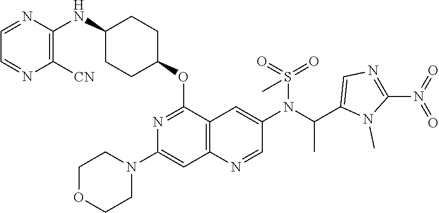 [SMILES: CC(c1cnc([N+](=O)[O-])n1C)N(c1cnc2cc(N3CCOCC3)nc(O[C@H]3CC[C@@H](Nc4nccnc4C#N)CC3)c2c1)S(C)(=O)=O]

Prepared using standard conditions for alkylation of a phenol to give product (5 mg)

1H NMR (500 MHz, DMSO-d6) δ 8.55-8.63 (m, 1H), 8.31-8.38 (m, 1H), 7.85-7.93 (m, 1H), 7.65-7.73 (m, 1H), 7.51-7.61 (m, 1H), 6.92 (s, 1H), 6.55 (s, 1H), 5.68-5.78 (m, 1H), 5.30-5.37 (m, 1H), 4.16-4.21 (m, 3H), 4.07-4.15 (m, 1H), 3.69-3.76 (m, 4H), 3.49-3.58 (m, 4H), 3.31 (s, 4H), 2.04-2.12 (m, 3H), 1.85-1.94 (m, 2H), 1.68-1.79 (m, 4H), 1.53-1.59 (m, 3H). LCMS: Rt=1.54 min, m/z at 678 MH+(MeCN, pH1).

### N-[(3-methyl-2-nitro-imidazol-4-yl)methyl]-N-[7-morpholino-5-[4-[[6-(trifluoromethyl)pyrazin-2-yl]amino]cyclohexoxy]-1,6-naphthyridin-3-yl]methanesulfonamide (LFA2101)

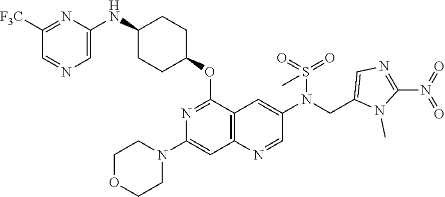 [SMILES: Cn1c(CN(c2cnc3cc(N4CCOCC4)nc(O[C@H]4CC[C@@H](Nc5cncc(C(F)(F)F)n5)CC4)c3c2)S(C)(=O)=O)cnc1[N+](=O)[O-]]

Prepared using standard conditions for alkylation of a phenol to give product (4 mg)

1H NMR (500 MHz, DMSO-d6) δ 8.74-8.78 (m, 1H), 8.23-8.27 (m, 1H), 8.13-8.17 (m, 1H), 8.04-8.09 (m, 1H), 7.74-7.80 (m, 1H), 6.94-7.01 (m, 1H), 6.51-6.54 (m, 1H), 5.29-5.36 (m, 1H), 5.10-5.16 (m, 2H), 3.92-3.97 (m, 3H), 3.84-3.91 (m, 1H), 3.69-3.76 (m, 4H), 3.47-3.54 (m, 4H), 3.21-3.26 (m, 3H), 1.99-2.08 (m, 2H), 1.83-1.91 (m, 4H), 1.71-1.79 (m, 2H).

LCMS: Rt=1.68 min., m/z at 707 MH+(MeCN, pH1).

### N-[5-[4-[(3-cyanopyrazin-2-yl)amino]cyclohexoxy]-7-morpholino-1,6-naphthyridin-3-yl]-N-[(3-methyl-2-nitro-imidazol-4-yl)methyl]methanesulfonamide (LFA2102)

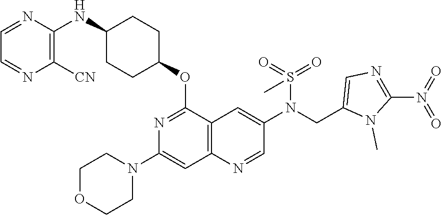 [SMILES: Cn1c(CN(c2cnc3cc(N4CCOCC4)nc(O[C@H]4CC[C@@H](Nc5nccnc5C#N)CC4)c3c2)S(C)(=O)=O)cnc1[N+](=O)[O-]]

Prepared using standard conditions for alkylation of a sulfonamide to give product (26 mg, 27%).

1H NMR (500 MHz, DMSO-d6) δ 8.76-8.80 (m, 1H), 8.35-8.38 (m, 1H), 8.11-8.14 (m, 1H), 7.90-7.93 (m, 1H), 7.35-7.40 (m, 1H), 6.97-7.00 (m, 1H), 6.49-6.54 (m, 1H), 5.36-5.41 (m, 1H), 5.11-5.15 (m, 2H), 4.04-4.12 (m, 1H), 3.95-3.98 (m, 3H), 3.70-3.74 (m, 4H), 3.49-3.52 (m, 4H), 3.28-3.31 (m, 3H), 2.06-2.13 (m, 2H), 1.74-1.88 (m, 6H). LCMS: Rt=1.5 min, m/z at 664 MH+(MeCN, pH1).

### N-[5-[4-[(5-cyanopyrazin-2-yl)amino]cyclohexoxy]-7-morpholino-1,6-naphthyridin-3-yl]-N-[(3-methyl-2-nitro-imidazol-4-yl)methyl]methanesulfonamide (LFA2103)

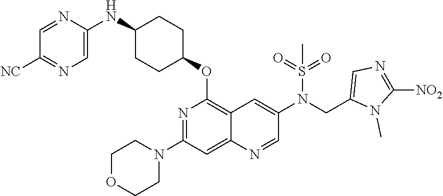 [SMILES: Cn1c(CN(c2cnc3cc(N4CCOCC4)nc(O[C@H]4CC[C@@H](Nc5cnc(C#N)cn5)CC4)c3c2)S(C)(=O)=O)cnc1[N+](=O)[O-]]

Prepared using standard conditions for alkylation of a sulfonamide to give product (1 mg, 3%).

1H NMR (500 MHz, CHLOROFORM-d) δ 8.81-8.84 (m, 1H), 8.28-8.31 (m, 1H), 8.06-8.10 (m, 1H), 7.84-7.88 (m, 1H), 7.01-7.04 (m, 1H), 6.94-7.00 (m, 1H), 6.50-6.54 (m, 1H), 5.33-5.41 (m, 1H), 4.96 (s, 2H), 4.08-4.16 (m, 4H), 3.82-3.88 (m, 4H), 3.58-3.65 (m, 4H), 3.11 (s, 3H), 2.20-2.27 (m, 2H), 1.90-1.96 (m, 2H), 1.79 (br. s., 2H), 1.44-1.53 (m, 2H).

LCMS: Rt=2.12 min., m/z at 664 MH+(MeOH, pH10).

### N-[5-[4-[[5-[2-(4-methylpiperazin-1-yl)ethoxy]pyrimidin-2-yl]amino]cyclohexoxy]-7-morpholino-1,6-naphthyridin-3-yl]methanesulfonamide (LFA2104)

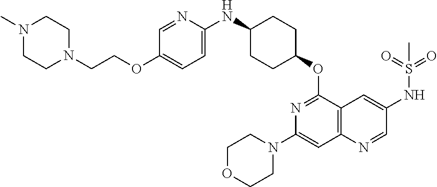 [SMILES: CN1CCN(CCOc2ccc(N[C@H]3CC[C@@H](Oc4nc(N5CCOCC5)cc5ncc(NS(C)(=O)=O)cc45)CC3)nc2)CC1]

A 20 mL microwave vial was charged with N-[5-(4-aminocyclohexoxy)-7-morpholino-1,6-naphthyridin-3-yl]methanesulfonamide (75.mg, 0.1800 mmol), 1-[2-[(6-chloro-3-pyridyl)oxy]ethyl]-4-methyl-piperazine (68.26 mg, 0.2700 mmol), Potassium tert-butoxide (59.9 mg, 0.5300 mmol) and tBuBrettPhos Pd G3 (15.2 mg, 0.0200 mmol). The vial was sealed and the mixture was purged with a stream of argon for 5 min. 1,4-Dioxane (3.5 mL) was added and the mixture was degassed by sparging with argon for 5 min. The mixture was heated to 100-C with stirring for 5 h. After cooling to RT the reaction mixture was filtered through Dicalite, eluting with DCM and MeOH. The filtrate was concentrated in vacuo. The resulting residue was purified by basic prep HPLC. The appropriate fractions were combined and the solvent removed at reduced pressure to afford N-[5-[4-[[5-[2-(4-methylpiperazin-1-yl)ethoxy]-2-pyridyl]amino]cyclohexoxy]-7-morpholino-1,6-naphthyridin-3-yl]methanesulfonamide (25.4 mg, 22.3% yield)methanol.

1H NMR (400 MHz, DMSO)—9.86 (s, 1H), 8.70 (d, J=2.7 Hz, 1H), 8.13 (d, J=2.7 Hz, 1H), 8.05 (s, 2H), 6.74-6.67 (m, 1H), 6.53 (s, 1H), 5.34 (s, 1H), 4.80-4.71 (m, 1H), 3.81-3.70 (m, 5H), 3.51-3.44 (m, 4H), 3.03 (s, 3H), 2.75-2.56 (m, 4H), 2.38-2.31 (m, 1H), 2.25 (s, 3H), 2.23-2.16 (m, 1H), 2.13-2.05 (m, 2H), 1.84-1.69 (m, 7H).

LC-MS (long, acidic method) rt 1.620 min, 599.2 [M+H]

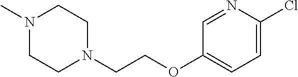 [SMILES: CN1CCN(CCOc2ccc(Cl)nc2)CC1]

2-(4-methylpiperazin-1-yl)ethanol (333.97 mg, 2.32 mmol) and Triphenylphosphine (809.91 mg, 3.09 mmol) were added to a stirred solution of 6-chloropyridin-3-ol (200.mg, 1.54 mmol) in THF (7.7196 mL) The mixture was ice-cooled before Di-tert-butylazodicarboxylate (533.26 mg, 2.32 mmol) was added in a single portion. The mixture was allowed to warm to RT and stirred for 19 h. The solvent was removed at reduced pressure. The resulting residue was purified by flash chromatography (20 g SiO2) eluting with a gradient 0-5% methanol in DCM. The appropriate fractions were combined and the solvent removed at reduced pressure to afford product as a pale yellow oil. NMR shows tBu Mitsunobu by-products. Mixture was taken up in methanol (2 mL) and Hydrogen chloride in dioxane (1.16 mL, 4.63 mmol) added. The reaction was allowed to stir overnight at room temperature. The reaction mixture was onto a 2 g SCX. The column was washed with methanol and eluted with 2M ammonia in methanol. The ammonia fraction was concentrated at reduced pressure to afford compound.

1H NMR (400 MHz, CDCl3)—8.06 (dd, J=2.7, 1.0 Hz, 1H), 7.26-7.16 (m, 2H), 4.12 (t, J=5.7 Hz, 2H), 2.82 (t, J=5.7 Hz, 2H), 2.70-2.45 (m, 8H), 2.31 (s, 3H). Product: 1-[2-[(6-chloro-3-pyridyl)oxy]ethyl]-4-methyl-piperazine (133 mg, 33.7% yield)

### Example 5: IC50 Data

### Study 1

### DNA-PK Assay for Biochemical Enzymatic Inhibition

A biochemical assay was performed to identify IC₅₀ values against DNA-PK activity in a two-step reaction with a kinase reaction followed by ADP Glo™ Kinase assay Kit (Promega V9102).

Three-fold serial dilutions of compounds in DMSO were prepared in kinase assay buffer (50 mM HEPES pH 7.5, 20 mM MgCl₂, 100 mM KCl, 50 μM DTT, 10 μg/ml calf thymus DNA, 0.01% Tween 20). Kinase assay buffer was used to prepare compound treatments to 0.02% DMSO (dimethyl sulphoxide) content. DMSO controls were prepared to produce a maximum and minimum luminescence signal for normalization. Substrate (Anaspec, AS-60210-5) and ATP (Promega V9102) and ATP mix was prepared by dilution in kinase assay buffer for 434.4 μM and 64.4 μM final assay concentrations, respectively. Compounds and control were added to 25 ng DNA-PK enzyme (Invitrogen, PR9107A) diluted in kinase buffer in assay plate (Corning 267459) and incubated for 15 minutes at room temperature prior to addition of substrate and ATP mix.

Throughout the assay, each time a reagent was added to the microplate, it was centrifuged for 1 minute at 1300 rcf. All reagents were added, and incubations carried out at room temperature. The reaction was then incubated for 60 minutes. ADP-ATP standards at 0, 4, 10, 40, 80, and 100% ADP (prepared as specified in the ADP Glo™ Kinase assay kit) were also added to the microplate at a volume equal to the total kinase reaction. The ADP Glo™ Kinase Assay kit was then used to quantify DNA-PK activity: following the 60-minute incubation, a 1:1 volume of ADP Glo reagent was added to all wells, then following a further 45-minute incubation, a 1:1 volume of Kinase Detection Reagent was added to all wells. After this two-step assay, plates were incubated in a Synergy-Neo2 plate reader for 30 minutes with gentle shaking, followed by endpoint luminescent read Luminescence data was normalized by subtracting the background signal and expressing all background-adjusted values as a percentage of the average maximum signal. The data was then expressed as percent inhibition by subtracting from 100% (i.e. maximum luminescent signal will be 0% inhibition) and the resulting data plotted against ten concentration points on a logarithmic scale, using GraphPad Prism. Non-linear regression analysis was performed on each concentration response curve and the log [inhibitor] vs response—variable slope (four parameters) equation was selected to generate IC50 values. Results of the DNA-PK Assay for Biochemical Enzymatic Inhibition by compounds are provided in the table.

### Study 2

### Cellular Assay for Inhibition of DNA-PK Phosphorylation at Serine 2056

FaDu cells were purchased from ATCC and maintained in MEM alpha medium (Life Technologies, cat. no. 12561) supplemented with 10% fetal bovine serum, and 1% GlutaMAX at 37° C. supplemented with 5% CO2. 50,000 cells were seeded to each well of a 96-well cell culture plate in 50 μL of culture medium and incubated for 16-24 hours. Three-fold serially diluted compounds were prepared in DMSO. Culture media was used to prepare compound treatments to 0.3% DMSO (dimethyl sulphoxide) content. Compound treatments were added to the cells for 1 hour prior incubation, followed by the addition of neocarzinostatin in fresh culture medium to 1 μg/mL final concentration for an additional 2 hours incubation to induce DNA DSBs (double-strand breaks). DMSO controls were incubated with fresh culture medium alone for the additional two hours incubation. The cells were then lysed and frozen at −20° C. Cell lysates were thawed on wet ice and protein concentrations quantified by BCA Protein Assay (Thermo #23225). Lysis buffer was used to normalize lysates by protein content and all lysates were then investigated by sandwich ELISA by means of DNA-PK-specific antibodies (Abcam ab44815: total DNA-PK; ab124918: phosphoserine-2056 DNA-PK) in sandwich ELISA format on the MSD S600MM platform (MesoScale Discovery). % inhibition was calculated for all data points: =1−(DATA_PT−AVERAGE(DMSO controls))/(AVERAGE(NCS controls)−AVERAGE(DMSO controls)). Logarithm of the molar concentration of compound was plotted against percent inhibition to calculate IC50 values, with curve fit settings for data fit to log(inhibitor) vs. normalized response—Variable slope using curve fit software (GraphPad Prism). IC50 values of the compounds are provided in the table below.

TABLE 1
IC50 Values of Biochemical Assay and
Cellular Assay for DNA-PK Inhibition

| | Biochemical Assay | Cellular Assay |
|---|---|---|
| Legend for Table 1 | IC₅₀ (nM) | IC₅₀ (μM) |
| IC₅₀ < 0.5 nM | A | |
| 0.5 nM ≤ IC₅₀ < 1 nM | B | |
| 1 nM ≤ IC₅₀ < 10 nM | C | |
| 10 nM ≤ IC₅₀ < 150 nM | D | |
| IC₅₀ < 0.1 μM | | A |
| 0.1 μM ≤ IC₅₀ < 1 μM | | B |
| 1 μM ≤ IC₅₀ < 10 μM | | C |
| 10 μM ≤ IC₅₀ < 30 μM | | D |

| | Biochemical Assay | Cellular Assay |
|---|---|---|
| Compound | IC50 (nM) | IC50 (μM) |
| 5 | C | D |
| 6 | D | B |
| 12 | D | |
| 13 | D | |
| 15 | C | B |
| 16 | C | C |
| 54 | B | A |
| 55 | A | B |
| 56 | A | B |
| 57 | D | D |
| 58 | A | B |
| 59 | A | D |
| 60 | A | A |
| 61 | B | A |
| 62 | A | A |
| 63 | A | A |
| 64 | A | B |
| 65 | A | A |
| 66 | B | A |
| 67 | A | A |
| 68 | A | B |
| 69 | A | A |
| 70 | A | A |
| 71 | A | A |
| 72 | C | A |
| 73 | B | B |
| 74 | B | B |
| 75 | A | A |
| 76 | A | B |
| 77 | A | B |
| 78 | B | B |
| 79 | B | B |
| 86 | A | A |
| 93 | D | C |
| 94 | D | B |
| 95 | B | A |
| 96 | A | A |
| 97 | A | A |
| 98 | A | A |
| 99 | A | A |
| 100 | A | A |
| 101 | A | A |
| 102 | A | A |
| 105 | C | B |
| 106 | D | |
| 107 | C | B |
| 117 | B | A |
| 125 | B | B |
| 126 | C | C |
| 129 | D | |
| 141 | A | A |
| 146 | C | C |
| 150 | B | B |
| 151 | C | B |
| 152 | B | B |
| 153 | C | B |
| 154b | D | |
| 154c | C | B |
| 154e | C | B |
| 156 | D | |
| 157 | C | C |
| 158 | D | B |
| 159 | D | |
| 163 | D | C |
| 164 | C | C |
| 165 | A | D |
| 166 | C | D |
| 167 | C | B |
| 168 | D | |
| 169 | D | C |
| 170 | D | |
| 171 | C | C |
| 172 | A | C |
| 173 | C | D |
| 174 | C | D |
| 175 | B | D |
| 178 | A | B |
| 179 | C | C |
| 180 | A | D |
| 181 | A | D |
| 182 | A | B |
| 183 | A | C |
| 186 | B | A |
| 190 | D | |
| 191 | C | B |
| 192 | A | B |
| 193 | B | B |
| 194 | A | B |
| 195 | D | C |
| 196 | C | B |
| 200b | A | B |
| 200c | D | D |
| 200d | C | B |
| 204a | C | B |
| 204b | A | A |
| 204c | C | A |

### Example 6: IC50 Data

Analysis performed in the same manner as IC50 study described above.

| Legend for Tables 2 and 3 | Biochemical Assay | Cellular Assay |
|---|---|---|
| below | IC₅₀ (nM) | IC₅₀ (μM) |
| IC₅₀ < 0.5 nM | A | |
| 0.5 nM ≤ IC₅₀ < 1 nM | B | |
| 1 nM ≤ IC₅₀ < 10 nM | C | |
| 10 nM ≤ IC₅₀ < 150 nM | D | |
| IC₅₀ < 0.1 μM | | A |
| 0.1 μM ≤ IC₅₀ < 1 μM | | B |
| 1 μM ≤ IC₅₀ < 10 μM | | C |
| 10 μM ≤ IC₅₀ < 30 μM | | D |

TABLE 2
IC50 Values of Biochemical Assay and
Cellular Assay for DNA-PK Inhibition

| | | Cellular |
|---|---|---|
| | Biochemical | Assay |
| | Assay IC50 | IC50 |
| Compound | (nM) | (μM) |
| LFA012 | A | B |
| LFA013 | D | C |
| LFA016 | D | D |
| LFA108 | D | D |
| LFA111 | B | B |
| LFA113 | A | A |
| LFA114 | D | D |
| LFA116 | D | D |
| LFA118 | D | D |

TABLE 3
IC50 Values of Cellular Assay for DNA-PK Inhibition

| | Cellular Assay |
|---|---|
| Compound | IC50 (μM) |
| LFA2001 | A |
| LFA2002 | B |
| LFA2003 | B |
| LFA2004 | B |
| LFA2005 | B |
| LFA2006 | A |
| LFA2007 | A |
| LFA2008 | A |
| LFA2009 | D |
| LFA2010 | B |
| LFA2011 | D |
| LFA2012 | A |
| LFA2013 | A |
| LFA2014 | B |
| LFA2015 | B |
| LFA2016 | B |
| LFA2017 | A |
| LFA2018 | A |
| LFA2019 | A |
| LFA2020 | A |
| LFA2021 | B |
| LFA2022 | A |
| LFA2023 | A |
| LFA2024 | B |
| LFA2025 | B |
| LFA2026 | B |
| LFA2027 | A |
| LFA2028 | A |
| LFA2029 | A |
| LFA2030 | A |
| LFA2031 | A |
| LFA2032 | A |
| LFA2033 | A |
| LFA2034 | A |
| LFA2035 | A |
| LFA2036 | A |
| LFA2037 | A |
| LFA2038 | A |
| LFA2039 | A |
| LFA2040 | A |
| LFA2041 | D |
| LFA2042 | D |
| LFA2043 | C |
| LFA2044 | C |
| LFA2045 | D |
| LFA2046 | D |
| LFA2047 | D |
| LFA2048 | D |
| LFA2049 | B |
| LFA2050 | C |
| LFA2051 | D |
| LFA2052 | C |
| LFA2053 | D |
| LFA2054 | D |
| LFA2055 | C |
| LFA2056 | C |
| LFA2057 | C |
| LFA2058 | C |
| LFA2059 | C |
| LFA2060 | D |
| LFA2061 | D |
| LFA2062 | C |
| LFA2063 | D |
| LFA2064 | C |
| LFA2065 | D |
| LFA2066 | D |
| LFA2067 | C |
| LFA2068 | C |
| LFA2069 | D |
| LFA2070 | D |
| LFA2071 | D |
| LFA2072 | C |
| LFA2073 | C |
| LFA2074 | C |
| LFA2075 | C |
| LFA2076 | C |
| LFA2077 | C |
| LFA2078 | A |
| LFA2079 | A |
| LFA2080 | A |
| LFA2081 | D |
| LFA2082 | D |
| LFA2083 | D |
| LFA2084 | D |
| LFA2085 | C |
| LFA2086 | D |
| LFA2087 | C |
| LFA2088 | C |
| LFA2089 | C |
| LFA2090 | D |
| LFA2091 | D |
| LFA2092 | D |
| LFA2093 | D |
| LFA2094 | D |
| LFA2095 | D |
| LFA2096 | D |
| LFA2097 | D |
| LFA2098 | B |
| LFA2099 | C |
| LFA2100 | D |
| LFA2101 | D |
| LFA2102 | D |
| LFA2103 | C |
| LFA2104 | B |
| LFA2105 | D |

### Example 7: EC50 Studies

Spheroid Data Set Summary Using pDNA-PK Inhibition & Cell Survival Endpoints for Effector Compounds.

Multicellular tumour spheroids determination of compound inhibition of pDNA-PK induction following radiation. Tumour spheroids were grown to approximately 0.5 mm in diameter and irradiated with 20 Gy in combination with inhibitor test compounds to determine their potency. Inhibitor concentration for 50% reduction in pDNA-PK induction (IC50) was determined via immunohistochemical staining in spheroid cryosections taken 2 hours following radiation.

Spheroid, Cell Line: HCT115, Immunohistochemical endpoint, pDNA-PK inhibition, EC50 in μm.

Spheroid+radiation pDNA-PK. Radiation: 20 Gy. Drug: serial dilution 5 or 10 μM down to 3.5 nM (11 wells). Spheroids: 3-6 spheroids per well (300-500 μm diameter). 20% 02 and 5% CO₂. 96 well plate format.

Protocol: pre-incubate 1 hours with drug, irradiate with 20 Gy, post-incubate 2 hours with drug, freeze and cryosection ×3 replicates.

Endpoint: fluorescent immunostin pDNA-PK ser2056 and image on microscope.

Analysis: determine EC50 concentration that decreases pDNA-PK induction by 50% after radiation.

EC50 ranges: A is 0.01 to 0.5 μM, B is 0.5 to 1.0 μM, C is 1.0 μM to 10 μM, and D is 10 μM to 100 μM.

| Compound | EC50 |
|---|---|
| 5 | C |
| 15 | C |
| 16 | D |
| 54 | A |
| 56 | B |
| 58 | B |
| 59 | D |
| 60 | A |
| 61 | A |
| 62 | B |
| 63 | C |
| 64 | A |
| 65 | C |
| 66 | C |
| 67 | B |
| 68 | B |
| 69 | C |
| 70 | C |
| 71 | A |
| 72 | B |
| 73 | C |
| 74 | C |
| 75 | B |
| 77 | B |
| 78 | C |
| 79 | C |
| 86 | A |
| 93 | D |
| 94 | C |
| 95 | A |
| 96 | B |
| 97 | A |
| 98 | A |
| 99 | A |
| 100 | A |
| 101 | A |
| 102 | A |
| 117 | B |
| 125 | C |
| 126 | D |
| 141 | A |
| 146 | C |
| 150 | B |
| 151 | C |
| 152 | C |
| 153 | D |
| 154c | C |
| 154e | C |
| 164 | C |
| 165 | D |
| 166 | D |
| 167 | D |
| 169 | D |
| 171 | C |
| 172 | C |
| 173 | D |
| 174 | D |
| 175 | D |
| 178 | B |
| 179 | D |
| 180 | D |
| 181 | D |
| 182 | A |
| 183 | C |
| 186 | C |
| 190 | D |
| 191 | D |
| 192 | A |
| 193 | C |
| 194 | C |
| 195 | D |
| 196 | C |
| 200b | C |
| 200c | D |
| 200d | C |
| 204a | C |
| 204b | A |
| 204c | C |

### Example 8: EC50 Studies

Spheroid Data Set Summary Using pDNA-PK Inhibition & Cell Survival Endpoints for Effector Compounds

Multicellular tumour spheroids determination of compound inhibition of pDNA-PK induction following radiation. Tumour spheroids were grown to approximately 0.5 mm in diameter and irradiated with 20 Gy in combination with inhibitor test compounds to determine their potency. Inhibitor concentration for 50% reduction in pDNA-PK induction (IC50) was determined via immunohistochemical staining in spheroid cryosections taken 2 hours following radiation. For cell survival endpoint 8 Gy radiation was used and 3 hours following radiation spheroids were dissociated, counted and plated for colony forming endpoint.

Spheroid+radiation pDNA-PK. Radiation: 20 Gy. Drug: serial dilution 5 or 10 μM down to 3.5 nM (11 wells). Spheroids: 3-6 spheroids per well (300-500 μm diameter). 20% 02 and 5% CO₂. 96 well plate format. Endpoint: fluorescent immunostain pDNA-PK ser2056 and image on microscope. Analysis: determine EC50 concentrations that decreases pDNA-PK induction by 50% after irradiation.

Spheroid+radiation colony forming cell survival assay (SF). Radiation 8 Gy. Drug: serial dilution 5 μM down to 10 nM (12 wells×2 repeat). Spheroids: 5-10 spheroids per well (300 to 500 μm diameter). HCT116. 5% 02 and 5% CO₂. 96 well pate format for assay. Protocol: pre incubate 1 hour with drug, irradiate 8 Gy, post incubate 3 hrs with drug, dissociate spheroids and count cells and plate for colony forming assay. Endpoint: wait 4-7 days and count colonies. Analysis: determine EC50 concentration that increase cell kill by 50% over radiation alone.

EC50 ranges: A is 0.01 to 0.5 μM, B is 0.5 to 1.0 μM, C is 1.0 μM to 10 μM, and D is 10 μM to 100 μM.

| | Cell line: HCT116 | Cell line: FaDU | Cell line: HCT116 |
|---|---|---|---|
| | Immunohistochemical | Immunohistochemical | Colony forming |
| | endpoint | endpoint | endpoint |
| | pDNA-PK inhibition | pDNA-PK inhibition | Surviving Fraction |
| Compound | EC50 (μM) | EC50 (μM) | EC50 (μM) |
| 5 | C | | |
| 15 | C | | |
| 16 | D | | |
| 54 | A | A | A |
| 64 | A | A | A |
| 150 | B | | |
| 151 | C | | |
| 152 | C | B | |
| 153 | D | | |
| 154c | C | | |
| 154e | C | B | |
| 164 | C | | |
| 165 | D | | D |
| 166 | D | | |
| 167 | D | | |
| 169 | D | | |
| 171 | C | | |
| 172 | C | C | |
| 173 | D | | |
| 174 | D | | |
| 175 | D | | D |
| 178 | B | C | |
| 179 | C | | |
| 183 | C | C | |

### Example 9: Prodrug Activation & EC50 Studies

### Study 1

### Prodrug Activation in Hypoxic Stirred Tumour Cell Suspensions

Prodrug compounds were assayed to determine their stability and rate of activation in stirred cell suspensions under 0.2 vs 5% oxygen. Compounds were diluted 1:1000 from DMSO stocks to 10 μM final concentration in tumour cell suspensions (1% by cell volume) in MEM media containing 10% FBS, 10 mM glucose & 3.7 g/L sodium bicarbonate. Cell lines assayed included A253, A549, Detroit562, FaDu, HCT116, HEP G2, HT29, SCC25, SCC9, SiHa & TC32. Compounds and cells were then incubated for 4 hours under constant mixing at 37° C. under 5% CO2 and either 0.2 or 5% O2. Following the incubation period cellular activation of the compounds was assayed via MeOH extraction and HPLC quantification. Data presented in the table below shows prodrug activation under hypoxic (O2% O2) versus stability under oxic (5% O2) conditions for compounds tested in the FaDu cell line.

Fraction of converted prodrug ranges: A is 0.15 to 1, B is 0.05 to 0.15, C is 0.02 to 0.05, and D is 0 to 0.02.

Fraction of prodrug converted
to active form
Cell line:
FaDu

| | Hypoxic | Oxic |
|---|---|---|
| Compound | 0.2% O2 | 5% O2 |
| LFA114 | A | B |
| LFA013 | D | D |
| LFA118 | A | C |
| LFA116 | A | C |
| LFA016 | C | D |
| LFA123 | A | B |
| LFA121 | A | B |
| LFA135 | D | D |
| LFA2060 | A | D |
| LFA2059 | B | D |
| LFA2058 | B | C |
| LFA2057 | A | B |
| LFA2056 | C | C |
| LFA2055 | A | D |
| LFA2054 | B | D |
| LFA2053 | B | D |
| LFA2052 | B | C |
| LFA2051 | A | D |
| LFA2050 | B | B |
| LFA2049 | A | B |
| LFA2070 | B | D |
| LFA2069 | A | C |
| LFA2068 | B | D |
| LFA2067 | B | B |
| LFA2066 | A | B |
| LFA2065 | A | C |
| LFA2064 | B | C |
| LFA2063 | A | B |
| LFA2062 | B | B |
| LFA2061 | B | D |
| LFA2071 | A | B |
| LFA2072 | B | B |
| LFA2073 | B | C |
| LFA2074 | B | C |
| LFA2075 | B | B |
| LFA2076 | B | B |
| LFA2077 | B | B |
| LFA2103 | B | D |
| LFA2048 | B | D |
| LFA2047 | B | D |
| LFA2046 | D | D |
| LFA2045 | D | D |
| LFA2009 | B | C |
| LFA2010 | B | C |
| LFA2011 | C | D |
| LFA2044 | C | D |
| LFA2043 | B | D |
| LFA2041 | A | B |
| LFA2042 | A | C |
| LFA2081 | B | B |
| LFA2082 | A | C |
| LFA2093 | B | B |
| LFA2094 | B | B |
| LFA2083 | A | C |
| LFA2084 | A | C |
| LFA2102 | C | D |
| LFA2101 | C | D |
| LFA2085 | B | D |
| LFA2086 | D | D |
| LFA2087 | A | D |
| LFA2091 | D | D |
| LFA2092 | D | D |
| LFA2100 | C | D |
| LFA2099 | B | D |
| LFA2098 | B | D |
| LFA2088 | B | D |
| LFA2089 | B | D |
| LFA2090 | D | D |
| LFA2105 | A | D |
| LFA2097 | C | D |

### Study 2

### Effector Radiation Sensitization in Multicellular Tumour Spheroids

Multicellular tumour spheroids were used to determine potency of test compounds in a 3D tissue environment using inhibition of pDNA-PK induction following radiation or cell survival as the endpoint. For the pDNA-PK endpoint HCT116 or FaDU tumour spheroids were grown to approximately 0.5 mm in diameter and irradiated with 20 Gy in combination with inhibitor test compounds to determine their potency. Experiments were carried out in 96 well plates containing 0.7 mls of MEM media containing 10% FBS and 10 mM glucose under 20% O2 & 5% CO2 at 37° C. Following dilution of compounds 1:1000 from DMSO stocks, compounds were serially diluted from 10 μM down to 3.5 nM and 5-10 spheroids added. 96 well plates were constantly stirred to avoid spheroids settling during the course of the experiment. The inhibitor concentration for 50% reduction in pDNA-PK induction (EC50) was determined via immunohistochemical staining in spheroid cryosections taken 2 hours following radiation using anti-pDNA-PK (ser 2056) and fixed immediately in formalin following sectioning. For cell survival endpoint 8 Gy radiation was used and, 3 hours following radiation, spheroids were dissociated, counted and plated for colony forming endpoint. Data presented in the table compound potency as EC50 [μM].

EC50 ranges: A is 0.01 to 0.5 μM, B is 0.5 to 1.0 μM, C is 1.0 μM to 10 μM, and D is 10 μM to 100 μM.

| | | | Spheroid |
|---|---|---|---|
| | | | Cell line: |
| | Spheroid | Spheroid | HCT116 |
| | Cell line: HCT116 | Cell line: FaDU | Colony forming |
| | Immunohistochemical | Immunohistochemical | endpoint |
| | endpoint | endpoint | Surviving |
| | pDNA-PK inhibition | pDNA-PK inhibition | Fraction |
| Compound | EC50 μM | EC50 μM | EC50 μM |
| 5 | C | | |
| 15 | C | | |
| 16 | D | | |
| 54 | A | A | A |
| 56 | B | | |
| 58 | B | | |
| 59 | D | | |
| 60 | A | | |
| 61 | A | | |
| 62 | B | | |
| 63 | C | | |
| 64 | A | A | A |
| 65 | C | | |
| 66 | C | | |
| 67 | A | | |
| 68 | B | | |
| 69 | C | | |
| 70 | C | | |
| 71 | A | | |
| 72 | B | | |
| 73 | C | | |
| 74 | C | | |
| 75 | B | | |
| 76 | C | | |
| 77 | B | | |
| 78 | C | | |
| 79 | C | | |
| 86 | A | | |
| 93 | D | | |
| 94 | C | | |
| 95 | A | | |
| 96 | B | | |
| 97 | A | | |
| 98 | A | | |
| 99 | A | | |
| 100 | A | | |
| 101 | A | | |
| 102 | A | | |
| 117 | B | | |
| 125 | C | | |
| 126 | D | | |
| 146 | C | | |
| 150 | B | | |
| 151 | C | | |
| 152 | C | B | |
| 153 | D | | |
| 154c | C | | |
| 154e | C | B | |
| 164 | C | | |
| 165 | D | | D |
| 166 | D | | |
| 167 | D | | |
| 169 | D | | |
| 171 | C | | |
| 172 | C | C | |
| 173 | D | | |
| 174 | D | | |
| 175 | D | | D |
| 178 | B | C | |
| 179 | D | | |
| 180 | D | | |
| 181 | D | | |
| 182 | A | | |
| 183 | C | C | |
| 186 | C | | |
| 190 | D | | |
| 191 | D | | |
| 192 | A | | |
| 193 | C | | |
| 194 | C | | |
| 195 | D | | |
| 196 | C | | |
| 200b | C | | |
| 200c | D | | |
| 200d | C | | |
| 204a | C | | |
| 204b | A | | |
| 204c | C | | |

### Study 3

### Prodrug Radiation Sensitization in Multicellular Tumour Spheroids

Prodrug compounds were assayed to determine their potency in 3D multicellular spheroids in media maintained under 20% vs 5% oxygen. Multicellular tumour spheroids were grown to approximately 0.5 mm in diameter and irradiated with 20 Gy in combination with inhibitor test compounds to determine their potency using HCT 116 and FaDu cell lines. Experiments were carried out in 96 well plates containing 0.7 mls of MEM media containing 10% FBS and 10 mM glucose under 5% CO2 at 37° C. Following dilution of compounds 1:1000 from DMSO stocks, compounds were serially diluted from 10 μM down to 3.5 nM and 5-10 spheroids added. 96 well plates were constantly stirred to avoid spheroids settling during the course of the experiment. Endpoints were pDNA-PK (ser 2056) or pH2AX (ser 139) status in spheroid cryosections taken 1 or 2 hours following radiation and fixed immediately in formalin. Using tumour cell lines deficient in ATM improved the assay signal to noise and gH2AX readout. Using this assay prodrug activity could be compared in the more oxygenated outer spheroid layers versus less oxygenated hypoxic central cell layers. Data presented in the table below shows prodrug compound EC50 [μM] compared to that of its effector in HCT116 ATM−/− multicellular tumour spheroids for inhibition of pH2AX induction following radiation.

EC50 ranges: A is 0.01 to 0.5 μM, B is 0.5 to 1 μM, C is 1 to 10 μM, and D is 10 to 100 μM.

| | Prodrug | Prodrug | Prodrug | Effector |
|---|---|---|---|---|
| | 20% O2 | 5% O2 | 5% O2 | 5% O2 |
| | Outer | Outer | Inner | Inner |
| | layers | layers | layers | layers |
| Compound | EC50 μM | EC50 μM | EC50 μM | EC50 μM |
| LFA118 | C | B | A | A |
| LFA116 | C | C | A | A |
| LFA114 | C | B | A | A |
| LFA013 | C | C | A | A |
| LFA016 | D | C | C | C |
| LFA123 | C | C | C | D |
| LFA108 | C | B | A | A |
| LFA121 | C | C | B | C |
| LFA135 | C | C | C | A |
| LFA2060 | D | C | B | B |
| LFA2059 | C | C | C | B |
| LFA2058 | C | A | A | A |
| LFA2057 | C | A | A | A |
| LFA2056 | C | A | A | A |
| LFA2055 | C | A | A | A |
| LFA2054 | D | B | A | A |
| LFA2053 | C | B | A | A |
| LFA2052 | C | B | A | A |
| LFA2051 | C | C | A | A |
| LFA2050 | C | C | A | A |
| LFA2049 | C | B | A | A |
| LFA2070 | D | C | A | A |
| LFA2069 | C | C | A | A |
| LFA2068 | D | C | B | B |
| LFA2067 | C | B | A | A |
| LFA2066 | C | C | A | A |
| LFA2065 | C | C | A | A |
| LFA2064 | C | C | A | B |
| LFA2063 | C | C | A | A |
| LFA2062 | C | B | A | B |
| LFA2061 | D | C | B | A |
| LFA2071 | C | C | A | A |
| LFA2072 | C | A | A | A |
| LFA2073 | C | C | A | A |
| LFA2074 | C | B | A | A |
| LFA2075 | C | B | A | A |
| LFA2076 | A | A | A | |
| LFA2077 | A | B | A | |
| LFA2103 | D | C | A | A |
| LFA2048 | B | C | B | A |
| LFA2047 | C | C | A | A |
| LFA2046 | C | C | B | |
| LFA2045 | C | C | C | |
| LFA2009 | C | C | C | A |
| LFA2010 | C | B | A | A |
| LFA2011 | C | C | C | A |
| LFA2044 | C | C | A | A |
| LFA2043 | C | B | A | A |
| LFA2041 | C | C | A | A |
| LFA2042 | C | C | A | A |
| LFA2081 | C | C | B | A |
| LFA2082 | C | C | A | A |
| LFA2093 | C | C | C | A |
| LFA2094 | D | C | B | A |
| LFA2083 | C | C | A | A |
| LFA2084 | D | C | A | A |
| LFA2102 | C | C | A | A |
| LFA2101 | D | D | D | D |
| LFA2085 | C | C | C | C |
| LFA2086 | C | C | C | C |
| LFA2087 | C | C | C | B |
| LFA2091 | D | D | D | C |
| LFA2092 | C | D | D | C |
| LFA2100 | D | C | A | A |
| LFA2099 | D | D | D | D |
| LFA2098 | C | C | C | B |
| LFA2088 | C | C | C | B |
| LFA2089 | C | C | C | C |
| LFA2090 | D | D | D | C |
| LFA2105 | D | C | C | A |
| LFA2097 | D | D | D | D |

### Example 10: Microsome Analysis

### Test 1: Microsomal Stability Assay

Stability in microsomes was determined using a 96-well format at Eurofins Discovery Services according to the following procedure. Mouse liver microsomes were obtained from a pool of 250 or more male CD-1 mice. Human liver microsomes were obtained from a pool of 50 or more donors of mixed gender. Final microsomal concentration was 0.1 mg/mL and test compound concentration was 0.1 uM with a maximum of 0.01% DMSO. The test compound was pre-incubated with pooled liver microsomes in phosphate buffer (pH 7.4) for 5 min in a 37° C. shaking water bath. The reaction was initiated by adding NADPH-generating system and incubated for 0, 15, 30, 45, and 60 min. The reaction was stopped by transferring the incubation mixture to acetonitrile/methanol. Samples were then mixed and centrifuged. Supernatants were used for HPLC-MS/MS analysis. The HPLC system consisted of a binary LC pump with autosampler, a C-18 column, and a gradient. Peak areas corresponding to the test compound were recorded. The compound remaining was calculated by comparing the peak area at each time point to time zero. The half-life was calculated from the slope of the initial linear range of the logarithmic curve of compound remaining (%) vs. time, assuming first order kinetics. In addition, the intrinsic clearance (Clint) was calculated from the half-life. Values for % compound remaining and Clint of test compounds are provided in the table below.

### Test 2: Heptacyte Stability Assay

Stability in hepatocytes was determined using a 96-well format at Eurofins Discovery Services according to the following procedure. Cryopreserved mouse hepatocytes were obtained from a pool of 10 or more male CD-1 mice. Cryopreserved human hepatocytes were obtained from a pool of 10 or more donors of mixed gender. Final hepatocyte density was 0.7 million viable cells per mL and test compound concentration was 1 uM with a maximum of 0.01% DMSO. Cryopreserved hepatocytes were thawed, washed, and resuspended in Krebs-Heinslet buffer (pH 7.3). The reaction was initiated by adding the test compound into cell suspension and incubated for 0, 30, 1, 1.5, and 2 h, respectively, at 37° C./5% C₀₂. The reaction was stopped by adding acetonitrile into the incubation mixture. Samples were then mixed, transferred completely to another 96-well plate, and centrifuged. Supernatants were used for HPLC-MS/MS analysis. The HPLC system consisted of a binary LC pump with autosampler, a C-18 column, and a gradient. Peak areas corresponding to the test compound were recorded. The compound remaining was calculated by comparing the peak area at each time point to time zero. The half-life was calculated from the slope of the initial linear range of the logarithmic curve of compound remaining (%) vs. time, assuming first order kinetics. In addition, the intrinsic clearance (Clint) was calculated from the half-life. Values for % compound remaining and Clint of test compounds are provided in the table below.

| | Microsomal | Hepatocyte |
|---|---|---|
| | Stability | Stability |
| Clᵢₙₜ < 116 uL/min/mg | A | |
| Clᵢₙₜ > 116 uL/min/mg | B | |
| Clᵢₙₜ < 8.2 uL/min/10⁶ cells | | C |
| Clᵢₙₜ > 8.2 uL/min/10⁶ cells | | D |
| % remaining = 0-25% | E | E |
| % remaining = 25-50% | F | F |
| % remaining = 50-75% | G | G |
| % remaining = 75-100% | H | H |

| | | | | | Hepatocyte | | | |
|---|---|---|---|---|---|---|---|---|
| | Microsomal | | | | Stability | | | |
| | Stability | | | | | % | | % |
| | | % | | % | | rem. | | rem. |
| | Mouse | rem. | Human | rem. | Mouse | 120 | Human | 120 |
| Compound | Clint | 60 min | Clint | 60 min | Clint | min | Clint | min |
| 5 | B | E | | | | | | |
| 12 | B | E | | | | | | |
| 15 | B | E | | | | | | |
| 54 | A | G | B | E | C | G | C | F |
| 55 | B | E | B | F | | | | |
| 56 | B | E | B | E | | | | |
| 58 | B | E | | | | | | |
| 60 | B | F | | | | | | |
| 61 | A | H | B | F | C | G | C | G |
| 62 | A | F | A | G | C | H | D | F |
| 63 | B | F | A | H | C | H | D | F |
| 64 | A | G | A | H | C | G | | |
| 65 | A | G | B | G | C | G | C | F |
| 66 | A | G | B | F | C | F | C | G |
| 67 | A | G | B | E | | | | |
| 68 | A | F | | | C | G | D | F |
| 69 | B | F | A | G | C | H | C | G |
| 70 | B | F | B | F | | | | |
| 71 | A | G | A | F | C | G | C | G |
| 72 | B | E | | | | | | |
| 75 | A | G | B | F | C | G | C | H |
| 76 | | | | | D | F | C | G |
| 86 | A | G | A | G | C | H | C | H |
| 95 | | | | | D | F | D | F |
| 96 | | | | | C | G | D | F |
| 97 | A | F | B | E | C | H | C | G |
| 98 | | | | | C | G | D | F |
| 99 | | | | | C | G | D | F |
| 100 | | | | | D | E | D | E |
| 101 | | | | | D | F | D | F |
| 102 | | | | | C | H | C | G |
| 117 | A | F | B | E | C | G | C | G |
| 125 | A | H | A | H | | | | |
| 141 | A | G | A | G | C | G | C | G |
| 146 | B | F | | | | | | |
| 150 | B | E | | | | | | |
| 154e | B | E | | | | | | |
| 156 | B | E | | | | | | |
| 157 | B | E | | | | | | |
| 158 | B | E | | | | | | |
| 165 | A | H | | | C | H | | |
| 166 | B | E | | | | | | |
| 167 | B | E | | | | | | |
| 171 | A | G | | | | | | |
| 172 | A | G | | | D | F | | |
| 175 | A | H | | | | | | |
| 182 | A | G | A | G | C | H | C | H |
| 183 | A | H | | | | | | |
| 186 | A | G | B | F | | | | |
| 192 | | | | | C | H | C | H |
| 193 | | | | | D | F | C | G |
| 200b | | | | | C | G | C | G |
| 204a | | | | | C | H | C | H |
| 204b | | | | | C | G | C | G |
| 204c | | | | | C | G | C | H |

### Example 11: Microsome Analysis

Analysis performed in the same manner as the microsome analysis described above.

| | Microsomal | Hepatocyte |
|---|---|---|
| | Stability | Stability |
| Clᵢₙₜ < 116 uL/min/mg | A | |
| Clᵢₙₜ > 116 uL/min/mg | B | |
| Clᵢₙₜ < 8.2 uL/min/10⁶ cells | | C |
| Clᵢₙₜ > 8.2 uL/min/10⁶cells | | D |
| % remaining = 0-25% | E | E |
| % remaining = 25-50% | F | F |
| % remaining = 50-75% | G | G |
| % remaining = 75-100% | H | H |

| | | | | | Hepatocyte | | | |
|---|---|---|---|---|---|---|---|---|
| | Microsomal | | | | Stability | | | |
| | Stability | | | | | % | | % |
| | | % | | % | | rem. | | rem. |
| | Mouse | rem. | Human | rem. | Mouse | 120 | Human | 120 |
| Compound | Clint | 60 min | Clint | 60 min | Clint | min | Clint | min |

| | | | | |
|---|---|---|---|---|
| LFA012 | A | H | A | H |
| LFA016 | A | G | A | F |
| LFA111 | A | F | A | G |
| LFA113 | A | F | A | H |
| LFA114 | B | E | A | E |

### Example 12: Traffic Light Reporter (TLR) Assay

### Traffic Light Reporter (TLR) Assay for HDR (Homology-Directed Repair) Efficiency

The HEK293-EGIP (Enhanced Green Fluorescent Inhibited Protein) stable cell line is purchased from System Biosciences (SBI). The HEK293-EGIP cell line harbors a disrupted GFP coding sequence with a stop codon and a 53-bp genomic fragment from the AAVS 1 locus. Cells are maintained in DMEM (Life Technologies, cat. no. 103 13-039) supplemented with 10% fetal bovine serum, and 1% GlutaMAX at 37° C. supplemented with 500 CO2.

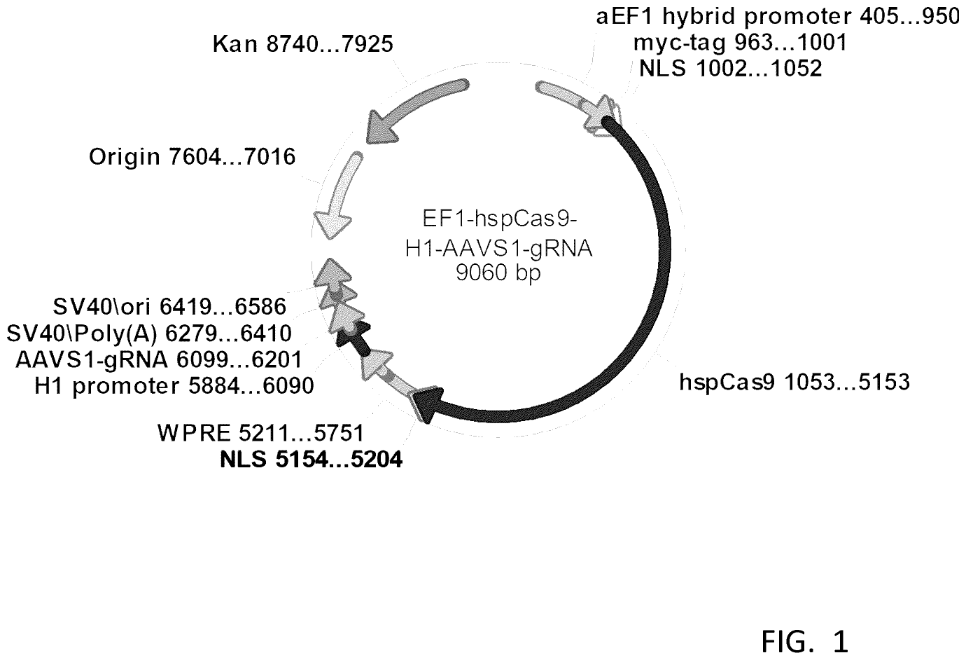

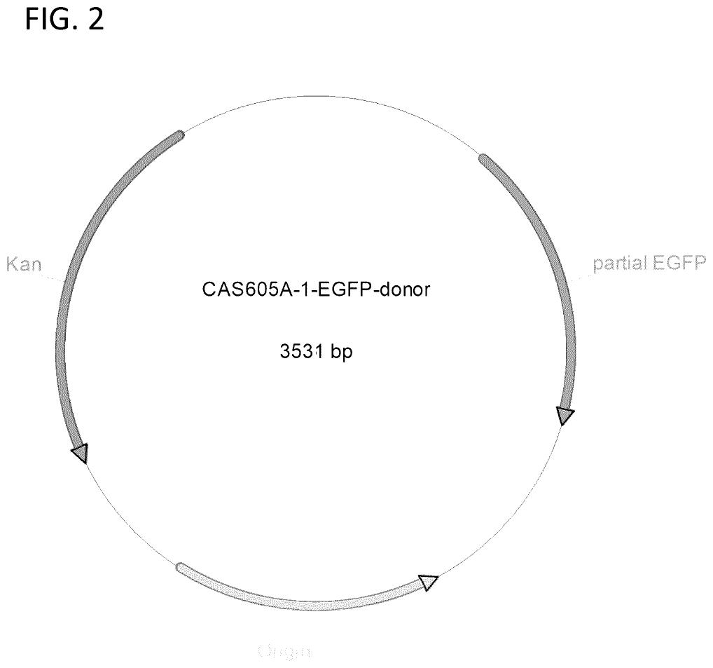

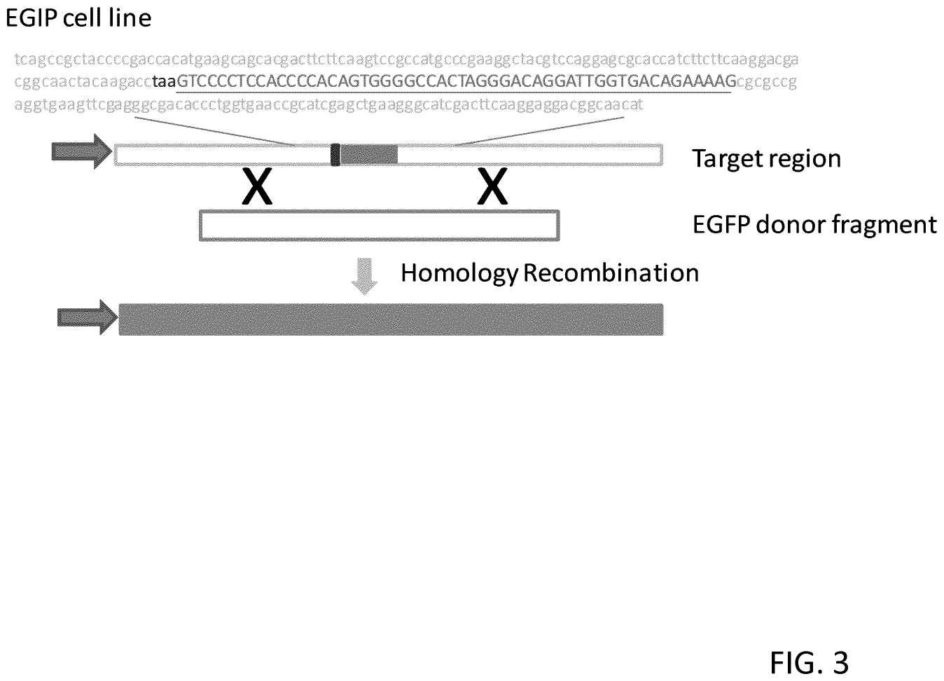

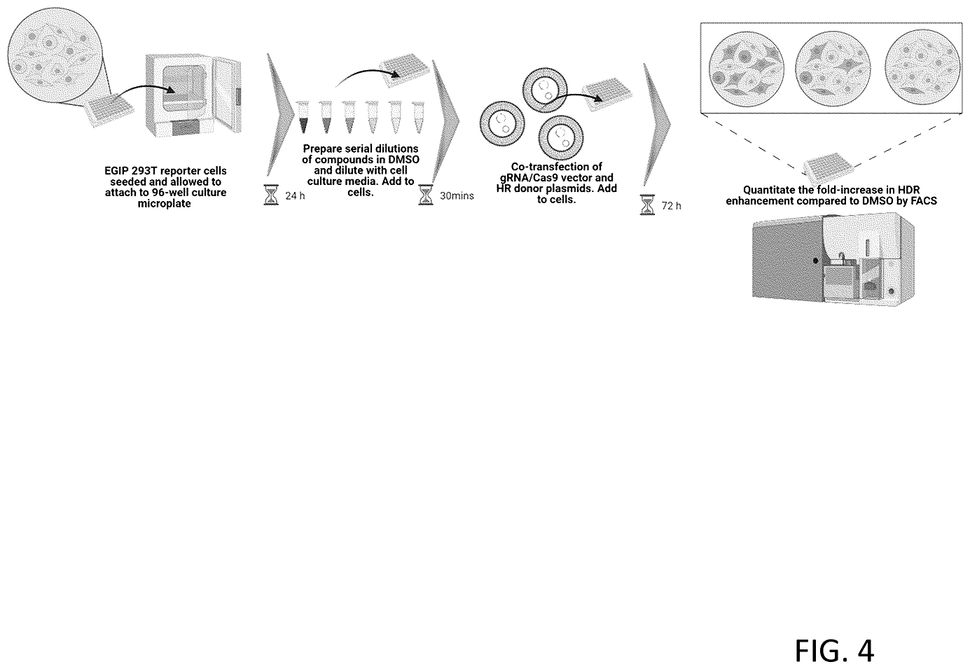

The HEK293-EGIP stable cells are incubated with 0.32% DMSO or serially diluted compounds for 30 minutes, followed by transfection with the two-in-one gRNA/CRISPR-Cas9 dual plasmid vector shown in FIG. 1, and plasmid repair donor shown in FIG. 2 (both plasmids from System Biosciences). Transfection is carried out using Lipofectamine 3000 (Invitrogen) following manufacturer's protocol. Transfected cells are incubated for 3 days followed by flow cytometry analysis to evaluate the amount increase in HDR of CRISPR-genome edited HEK-EGIP cells in comparison to the DMSO vehicle gRNA-Cas9 and donor template condition. In the TLR system, the HEK293-EGIP stable cell line expressing the “broken” green fluorescent protein eGFP, relies on HDR-mediated repair to generate functional eGFP in the presence of DNA donor template (see FIGS. 3 and 4). As shown in the experimental workflow in FIG. 4, functional GFP positive cells appear through HDR pathway where the 56 nt insertion is replaced with the correct DNA sequence in which the 56 nt insertion is absent. Forty-eight hours post-transfection through lipofection, GFP positive cells will usually emerge. Flow cytometry analysis is conducted at 72 hours. To assess potential toxicity of dual expression gRNA-Cas9 with donor template and culture of the cells with DMSO or in the presence of compounds, cells are incubated with SYTOX™ Red Dead Cell Stain (Invitrogen S34859) as per manufacturer's recommended protocol prior to flow cytometry analysis. HDR efficiency is determined as the fold-increase in enhancement of the DNA repair process using the CRISPR-Cas9 system in the presence of a donor repair template with compound relative to DMSO, as indicated by percentage of viable GFP positive cells by Sytox Red low signal and GFP high signal in flow cytometry analysis.

FIG. 1 shows the design of the two-in-one gRNA/CRISPR-Cas9 dual plasmid vector.

FIG. 2 shows the design of donor template plasmid vector.

FIG. 3 shows the cell line, and the targeted polynucleotide region, used in the traffic light reporter assay for monitoring HDR efficiency.

FIG. 4 shows the experiment workflow used in the traffic light reporter assay for monitoring HDR efficiency.

### Example 13: Crispr Inactivation of DNA-PK to Demonstrate Direct Activity of DNA-PK Inhibitors on HDR (Homology Directed Repair)

This assay is established to demonstrate that any increased HDR activity is directly due to DNA-PK inhibition. Genome editing positive control EGIP 293T cell lines (System Biosciences, Cat #: CAS606A-1) expressing eGFP with a premature stop codon in the AAVS1 locus are used to do the CRISPR. Ribonucleoprotein (RNP) CRISPR Cas9 gene editing is used to mutate the Lysine (K) 3752 for an Arginine (R), which has been previously shown to be critical for ATP-binding within the kinase site of DNA-PK (Kurimasa et al., Mol. Cell. Biol., 1999). Briefly, RNP is made by incubating the guide RNA with the Cas9 Nuclease at room temperature for 15 min. Cells are trypsinized for 5 min at 37° C., 1-2×10⁶ cells are centrifuged at 300×g for 5 min, washed with PBS, re-centrifuged and resuspended in Nucleofector solution (Lonza, SF Cell line X kit, Cat #: V4XC-2012). Transfection mix—containing the RNP complex, HDR donor oligo, Alt-R Cas9 Electroporation enhancer (IDT, Cat #: 1075916) and cell suspension—is electroporated using the Lonza 4D-Nucleofector X-Unit, program CM-130. Cells are immediately plated with Alt-R HDR enhancer V2 (IDT, Cat #: 10007910). Cells are sorted using a BD FACS Aria Fusion at McGill Goodman Cancer Institute, Flow Cytometry Core Facility. Single-cell colonies are then tested for K3752>R mutation using qPCR probes and sequencing. Once a single-cell colony expressing the R3752 mutation is found, cells are used in the cellbased assay and FACS analysis described below.

CRISPR edited EGIP cells with inactive DNA-PK are plated in 96-well plate at 8,000 cells/wells and incubated at 37° C. with 5% CO₂ for 48 h. Cells are co-transfected with hspCas9 plasmid containing a guide RNA targeting the AAVS1 locus (System Biosciences, Cat #: CAS601A-1), as well as a plasmid containing the corrective eGFP homologous recombinant donor sequence. This allows homology directed repair (HDR) pathway to remove the premature stop codon from eGFP, thus restoring the fluorescence. Following an 18 h incubation period post-transfection, cells are then incubated with a compound or vehicle for 24 h. Next, medium is changed and incubation is continued for 72 h before cells are harvested and FACS analysis is performed.

A BD LSRFortessa X-20 cell analyzer is used to determine the proportion of cells that underwent HDR repair of eGFP. Control cells (un-transfected) are used to set the FSC and SSC value, and Heat killed cells (SYTOX Red, ThermoFisher, Cat #: S34859) and GFP positive cells are used as controls. All samples are run for a total of 20,000 events at a flow rate of 0.5 ul/s. The efficacy of each compound is determined by the HDR rate indicated by eGFP positive cells, while percentage of cell death is monitored through SYTOX Red positive cells.

### Example 14: Pharmacokinetic Studies

### In Vivo Procedures

Pharmacokinetic analysis of compounds was conducted following bolus intravenous (IV) or bolus oral (PO) gavage in mice. Female mice (C57BL/6) in the 17-26 g range were weighed, and then administered an individually prescribed dose volume based on body weight of formulated compound via the intravenous (IV; tail vein) or oral gavage (PO) route (IV 5 mL/kg; PO 10 mL/kg). At selected timepoints post administration, blood was collected into vials with anticoagulant, processed for plasma by centrifugation, and then kept frozen (20° C.) until analysis.

Pharmacokinetic studies were conducted to estimate plasma concentrations obtained after oral gavage (PO) and intravenous (IV) single administration of compounds administered to male Sprague-Dawley rats. Six (6) rats were administered for each compound, route and dose, and plasma was collected at 12 timepoints over a 24 hr period for IV, and 12 timepoints over a 48 hr period for PO. Note that plasma was collected from only 3 animals per timepoint (n=3 for timepoints up to 10 hours, and n=3 for timepoints from 16 h to end of study). Dosing volumes were 5 mL/kg for IV and 10 mL/kg for PO gavage. IV compounds for administration were in solutions, whereas PO compounds were in solution or suspension.

Pharmacokinetic studies were conducted to estimate plasma concentrations obtained after oral gavage (PO) and intravenous (IV) single administration of compounds administered to separate groups of male Beagle dogs. Three (3) dogs were administered a compound via the intravenous (IV) route at 3 mg/kg and 2.5 mL/kg, and plasma was collected at 12 timepoints over a 24-hour period. Following a 7 days washout period, the same dogs were administered the same compound via PO at 10 mg/kg and 5 mL/kg, and plasma was collected at 12 timepoints over a 48-hour period.

### Description of the Bioanalytical Method

The in-vivo study plasma samples were analyzed using Ultra Performance Liquid Chromatography (UPLC) with tandem mass spectrometry (MS/MS) detection in multiple reaction monitoring (MRM) mode. A generic bioanalytical method involving plasma sample extraction and cleanup by protein precipitation with acetonitrile was used followed by UPLC-MS/MS analysis.

A brief example of a generic bioanalytical UPLC-MS/MS method used for analysis of compounds in the in-vivo study plasma samples is as follows. Plasma samples were spiked with internal standard prior to extraction and cleanup by protein precipitation with ice cold acetonitrile containing 0.1% (v/v) ammonium hydroxide at a ratio of 3:1 (solvent:plasma) and centrifuged at 18213 rcf for 15 minutes at 4° C. to pellet precipitates. Aliquots of supernatants were transferred to a 96-well plate and evaporated to dryness at 40° C. under a stream of nitrogen gas. Samples were reconstituted in 50:50 acetonitrile/10 mM ammonium acetate buffer pH 8, mixed thoroughly and analyzed using Ultra Performance Liquid Chromatography (UPLC) with tandem mass spectrometry (MS/MS) detection in multiple reaction monitoring (MRM) mode. Matrix-matched calibration standards and quality control (QC) samples were prepared in naïve C57Bl/6 mouse plasma. The UPLC column used was Waters Acquity UPLC BEH C18 1.7 um, 2.1 mm×50 mm (with guard column). The UPLC mobile phase consisted of: Mobile phase A: 10 mM ammonium acetate in Water pH 8, and Mobile phase B: Acetonitrile. Separation was accomplished with a linear gradient of 5% mobile phase B to 95% mobile phase B in 2 min, and a flow rate of 0.35 mL/min. The MS/MS data acquisition was accomplished using 3 MRM Transitions in ESI+ mode. Parent ion mass 508.37 m/z-to-Daughter ions (m/z) 117.05, 232.13 and 270.22. The quantitation of concentrations in test samples was achieved by matrix-matched calibration with normalization using an internal standard. Data acquisition and analysis were performed using Waters MassLynx software with TargetLynx application manager.

### PK Analysis

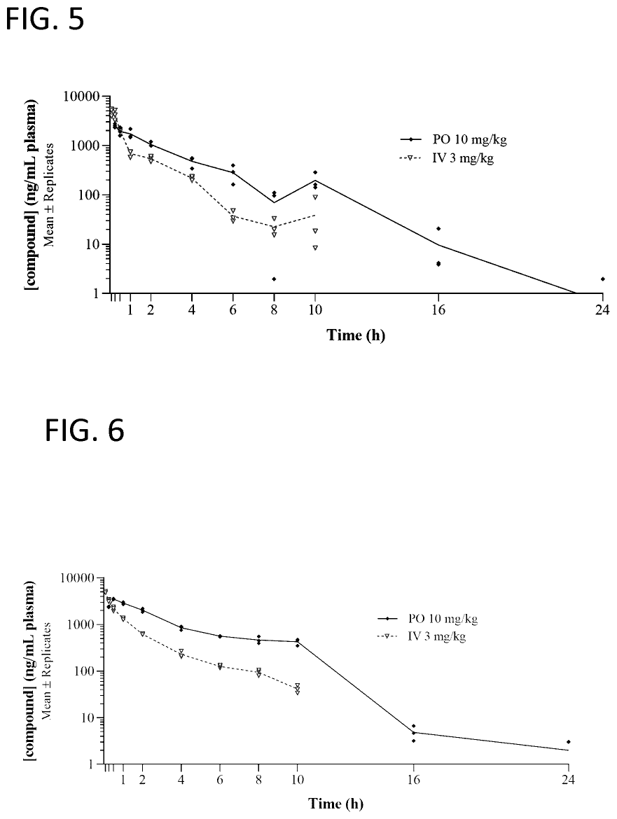

Estimation of PK parameters was performed using independent noncompartmental model method by PHOENIX WinNonlin software. Pharmacokinetic parameters resulting from this analysis for the compounds are provided in the tables below. Plasma concentrations for studies conducted in mice were plotted across post injection time as shown in FIGS. 5, 6, 7, 8, 9 and 10.

The results are described and shown below.

TABLE A
Pharmacokinetic parameters resulting from non-compartmental
analysis (NCA) of Compound 97 administered to Mice

| Compound | | Compound 97 | |
|---|---|---|---|
| Sample Type | | Mouse Plasma | Mouse Plasma |
| Route of Admin. | | IV | PO |
| Vehicle | | 5% w/v HPbCD, 0.7% w/v | 20% w/v HPbCD, |
| | | sodium chloride, acetate buffer | acetate buffer |
| | | (10 mM, pH 4) | (10 mM, pH 4) |
| Dose | (mg/kg) | 3 | 10 |
| Cₘₐₓ (5 min) | (ng/mL) | 4861 | 2524 |
| T_{1/2} | (h) | 2.21 | 2.07 |
| AUCₗₐₛₜ | (h*ng/mL) | 4341.59 | 6771.92 |
| AUC_{INF}_{_{—}}_{obs} | (h*ng/mL) | 4375.08 | 6773.87 |
| AUC_{_{—}}_{% Extrap}_{_{—}}_{obs} | (%) | 0.77 | 0.03 |
| Vz_obs | (mL/kg) | 2187.41 | 4415.23 |
| Cl_obs | (mL/h/kg) | 685.7 | 1476.26 |
| Rsq | | 0.98 | 0.98 |
| F | % | — | 47% |

FIG. 5 shows Plasma concentrations across time following administration of compound 97 as a single bolus via IV and PO gavage to mice. N═3/timepoint. Timepoints include 5 min (IV only), 15 min, 30 min, 1 h, 2 h, 4 h, 6 h, 8 h, 10 h, 16 h (PO only), 24 h (PG only).

TABLE B
Pharmacokinetic parameters resulting from non-compartmental
analysis (NCA) of Compound 61 administered to Mice

| Compound | | Compound 61 | |
|---|---|---|---|
| Sample Type | | Mouse Plasma | Mouse Plasma |
| Route of Admin. | | IV | PO |
| Vehicle | | 5% w/v HPbCD, 0.7% w/v | 20% w/v HPbCD, |
| | | sodium chloride, acetate buffer | acetate buffer |
| | | (10 mM, pH 4) | (10 mM, pH 4) |
| Dose | (mg/kg) | 3 | 10 |
| Cₘₐₓ (5 min) | (ng/mL) | 4896.2 | 3505.1 |
| T_{1/2} | (h) | 2.2 | 2.05 |
| AUCₗₐₛₜ | (h*ng/mL) | 5209.51 | 12558.78 |
| AUC_{INF}_{_{—}}_{obs} | (h*ng/mL) | 5340.47 | 12564.73 |
| AUC_{_{—}}_{% Extrap}_{_{—}}_{obs} | (%) | 2.45 | 0.05 |
| Vz_obs | (mL/kg) | 1784.9 | 2356.41 |
| Cl_obs | (mL/h/kg) | 561.75 | 795.88 |
| Rsq | | 0.97 | 0.93 |
| F | % | — | 72% |

FIG. 6 shows Plasma concentration across time following administration of compound 61 as a single bolus via IV and PG gavage to mice. n=3/timepoint. Timepoints include 5 min (IV only), 15 min, 30 min, 1 h, 2 h, 4 h, 6 h, 8 h, 10 h, 16 h (PO only), 24 h (PO only).

TABLE C
Pharmacokinetic parameters resulting from non-compartmental
analysis (NCA) of Compound 61 administered to Rat 61

| Compound | | Compound 61 | | | |
|---|---|---|---|---|---|
| Sample Type | | Rat Plasma | Rat Plasma | Rat Plasma | Rat Plasma |
| Route of Admin. | | IV | PO | PO | PO |
| Vehicle | | 8% w/v HPbCD, | 0.5% w/v HPMC 0.2% w/v Tween80, acetate buffer | | |
| | | 0.7% w/v sodium | (10 mM, pH 4) | | |
| | | chloride, acetate | | | |
| | | buffer (10 mM, | | | |
| | | pH 4) | | | |
| Dose | (mg/kg) | 3 | 3 | 10 | 30 |
| Cₘₐₓ (5 min) | (ng/mL) | 1602.20 | 87.45 | 234.97 | 655.25 |
| T_{1/2} | (h) | 1.49 | 5.68 | 2.64 | 13.74 |
| AUCₗₐₛₜ | (h*ng/mL) | 912.15 | 241.46 | 1071.23 | 5762.81 |
| AUC_{INF}_{_{—}}_{obs} | (h*ng/mL) | 913.06 | 329.86 | 1222.74 | 15790.22 |
| AUC_{_{—}}_{% Extrap}_{_{—}}_{obs} | (%) | 0.10 | 26.80 | 12.39 | 63.5 |
| Vz_obs | (mL/kg) | 7056.79 | 74544.30 | 31192.00 | 37654.53 |
| Cl_obs | (mL/h/kg) | 3285.66 | 9094.86 | 8178.38 | 1899.91 |
| Rsq | | 1.00 | 0.90 | 0.95 | 0.76 |
| F | % | — | 26% | 35% | 63% |

TABLE D
Pharmacokinetic parameters resulting from non-compartmental
analysis (NCA) of Compound 61 administered to Dog

| Compound | | Compound 61 | |
|---|---|---|---|
| Sample Type | | Dog Plasma | Dog Plasma |
| Route of Admin. | | IV | PO |
| Vehicle | | 15% w/v HPbCD, 0.5% w/v | 0.5% w/v HPMC 0.2% w/v |
| | | NaCl, acetate buffer | Tween80, acetate buffer |
| | | (10 mM, pH 5.5) | (10 mM, pH 4) |
| Dose | (mg/kg) | 3 | 10 |
| Cₘₐₓ (5 min) | (ng/mL) | 3700.00 | 1073.89 |
| T_{1/2} | (h) | 3.64 | 4.39 |
| AUCₗₐₛₜ | (h*ng/mL) | 3475.64 | 5297.04 |
| AUC_{INF}_{_{—}}_{obs} | (h*ng/mL) | 3501.20 | 5343.12 |
| AUC_{_{—}}_{% Extrap}_{_{—}}_{obs} | (%) | 0.79 | 0.78 |
| Vz_obs | (mL/kg) | 4480.58 | 11528.97 |
| Cl_obs | (mL/h/kg) | 860.29 | 2148.05 |
| Rsq | | 0.98 | 1.00 |
| F | % | — | 46% |

TABLE E
Pharmacokinetic parameters resulting from non-compartmental
analysis (NCA) of Compound 86 administered to Mice

| Compound | | Compound 86 | |
|---|---|---|---|
| Sample Type | | Mouse Plasma | Mouse Plasma |
| Route of Admin. | | IV | PO |
| Vehicle | | 5% w/v HPbCD, 0.7% w/v | 20% w/v HPbCD, |
| | | sodium chloride, acetate buffer | acetate buffer |
| | | (10 mM, pH 4) | (10 mM, pH 4) |
| Dose | (mg/kg) | 3 | 10 |
| Cₘₐₓ (5 min) | (ng/mL) | 6454.1 | 588.63 |
| T_{1/2} | (h) | 1.77 | 2.92 |
| AUCₗₐₛₜ | (h*ng/mL) | 4099.2 | 2619.3 |
| AUC_{INF}_{_{—}}_{obs} | (h*ng/mL) | 4158.9 | 2631.8 |
| AUC_{_{—}}_{% Extrap}_{_{—}}_{obs} | (%) | 1.44 | 0.47 |
| Vz_obs | (mL/kg) | 1842.6 | 16019 |
| Cl_obs | (mL/h/kg) | 721.34 | 3799.7 |
| Rsq | | 0.9 | 0.95 |
| F | % | — | 19% |

FIG. 7 shows Plasma concentration across time following administration of compound 86 as a single bolus via IV and PO gavage to mice. n=3/timepoint. Timepoints include 5 min (IV only), 15 min, 30 min, 1 h, 2 h, 4 h, 6 h, 8 h, 10 h, 16 h (PO only), 24 h (PO only).

TABLE F
Pharmacokinetic parameters resulting from non-compartmental
analysis (NCA) of Compound 86 administered to Rat

| Compound | | Compound 86 | | | |
|---|---|---|---|---|---|
| Sample Type | | Rat Plasma | Rat Plasma | Rat Plasma | Rat Plasma |
| Route of Admin. | | IV | PO | PO | PO |
| Vehicle | | 8% w/v HPbCD, | 0.5% w/v HPMC 0.2% w/v Tween80, acetate buffer | | |
| | | 0.7% w/v sodium | (10 mM, pH 4) | | |
| | | chloride, acetate | | | |
| | | buffer (10 mM, | | | |
| | | pH 4) | | | |
| Dose | (mg/kg) | 3 | 3 | 10 | 30 |
| Cₘₐₓ (5 min) | (ng/mL) | 886.14 | 28.20 | 203.18 | 1598.17 |
| T_{1/2} | (h) | 0.67 | 2.59 | 2.39 | 1.26 |
| AUCₗₐₛₜ | (h*ng/mL) | 370.80 | 71.60 | 1293.88 | 14070.61 |
| AUC_{INF}_{_{—}}_{obs} | (h*ng/mL) | 374.19 | 78.39 | 1488.60 | 14071.24 |
| AUC_{_{—}}_{% Extrap}_{_{—}}_{obs} | (%) | 0.91 | 8.66 | 13.08 | 0.0 |
| Vz_obs | (mL/kg) | 7725.22 | 142794.45 | 23132.91 | 3877.55 |
| Cl_obs | (mL/h/kg) | 8017.38 | 38270.65 | 6717.70 | 2132.01 |
| Rsq | | 0.95 | 1.00 | 0.97 | 0.87 |
| F | % | — | 19% | 105% | 379% |

TABLE G
Pharmacokinetic parameters resulting from non-compartmental
analysis (NCA) of Compound 86 administered to Dog

| Compound | | Compound 86 | |
|---|---|---|---|
| Sample Type | | Dog Plasma | Dog Plasma |
| Route of Admin. | | IV | PO |
| Vehicle | | 15% w/v HPbCD, 0.5% w/v | 0.5% w/v HPMC 0.2% w/v |
| | | NaCl, acetate buffer | Tween80, acetate buffer |
| | | (10 mM, pH 5.5) | (10 mM, pH 4) |
| Dose | (mg/kg) | 3 | 10 |
| Cₘₐₓ (5 min) | (ng/mL) | 1732.17 | 1539.70 |
| T_{1/2} | (h) | 13.11 | 2.62 |
| AUCₗₐₛₜ | (h*ng/mL) | 1734.21 | 6093.61 |
| AUC_{INF}_{_{—}}_{obs} | (h*ng/mL) | 1847.89 | 6127.20 |
| AUC_{_{—}}_{% Extrap}_{_{—}}_{obs} | (%) | 5.80 | 0.51 |
| Vz_obs | (mL/kg) | 28995.60 | 6548.71 |
| Cl_obs | (mL/h/kg) | 1697.24 | 1718.38 |
| Rsq | | 1.00 | 0.94 |
| F | % | — | 105% |

TABLE H
Pharmacokinetic parameters resulting from non-compartmental
analysis (NCA) of Compound 75 administered to Mice

| Compound | | Compound 75 | |
|---|---|---|---|
| Sample Type | | Mouse Plasma | Mouse Plasma |
| Route of Admin. | | IV | PC |
| Vehicle | | 5% w/v HPbCD, 0.7% w/v | 20% w/v HPbCD, |
| | | sodium chloride, acetate buffer | acetate buffer |
| | | (10 mM, pH 4) | (10 mM, pH 4) |
| Dose | (mg/kg) | 3 | 10 |
| Cₘₐₓ (5 min) | (ng/mL) | 6018.87 | 5588.83 |
| T_{1/2} | (h) | 1.67 | 2.17 |
| AUCₗₐₛₜ | (h*ng/mL) | 7959.12 | 18233 |
| AUC_{INF}_{_{—}}_{obs} | (h*ng/mL) | 8174.2 | 18246.08 |
| AUC_{_{—}}_{% Extrap}_{_{—}}_{obs} | (%) | 2.63 | 0.07 |
| Vz_obs | (mL/kg) | 885.15 | 1715.97 |
| Cl_obs | (mL/h/kg) | 367.01 | 548.06 |
| Rsq | | 0.94 | 1 |
| F | % | — | 69% |

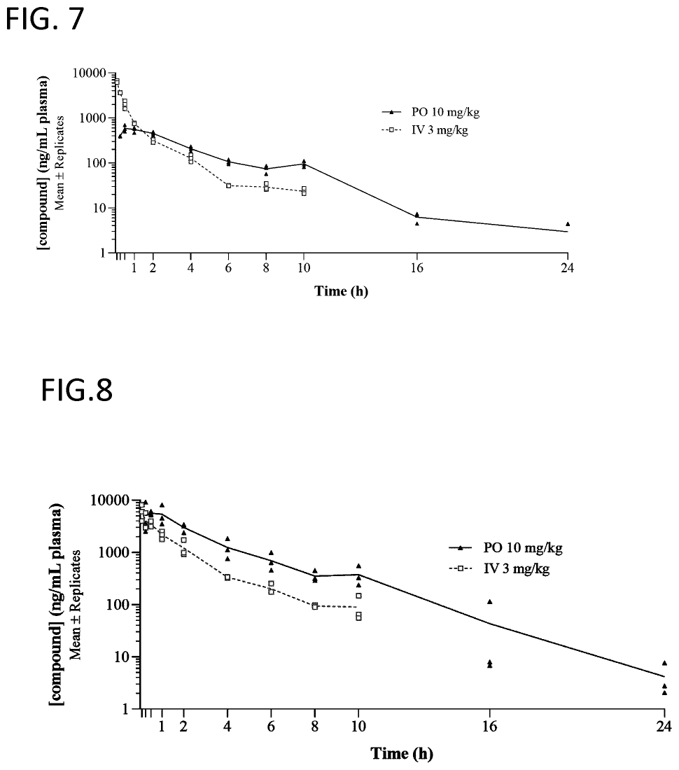

FIG. 8 shows Plasma concentration across time following administration of compound 75 as a single bolus via IV and PO gavage to mice. n=3/timepoint. Timepoints include 5 min IV only), 15 min, 30 min, 1 h, 2 h, 4 h, 6 h, 8 h, 10 h, 16 h (PO only), 24 h (PO only).

TABLE I
Pharmacokinetic parameters resulting from non-compartmental
analysis (NCA) of Compound 69 administered to Mice

| Compound | | Compound 69 | |
|---|---|---|---|
| Sample Type | | Mouse Plasma | Mouse Plasma |
| Route of Admin. | | IV | PO |
| Vehicle | | 5% w/v HPbCD, 0.7% w/v | 20% w/v HPbCD, |
| | | sodium chloride, acetate buffer | acetate buffer |
| | | (10 mM, pH 4) | (10 mM, pH 4) |
| Dose | (mg/kg) | 3 | 10 |
| Cₘₐₓ (5 min) | (ng/mL) | 611.13 | 548.91 |
| T_{1/2} | (h) | 4.63 | 5.4 |
| AUCₗₐₛₜ | (h*ng/mL) | 1627.65 | 5675.65 |
| AUC_{INF}_{_{—}}_{obs} | (h*ng/mL) | 2269.49 | 6074.4 |
| AUC_{_{—}}_{% Extrap}_{_{—}}_{obs} | (%) | 28.28 | 6.56 |
| Vz_obs | (mL/kg) | 8824.88 | 12835.77 |
| Cl_obs | (mL/h/kg) | 1321.88 | 1646.25 |
| Rsq | | 0.87 | 0.9 |
| F | % | — | 105% |

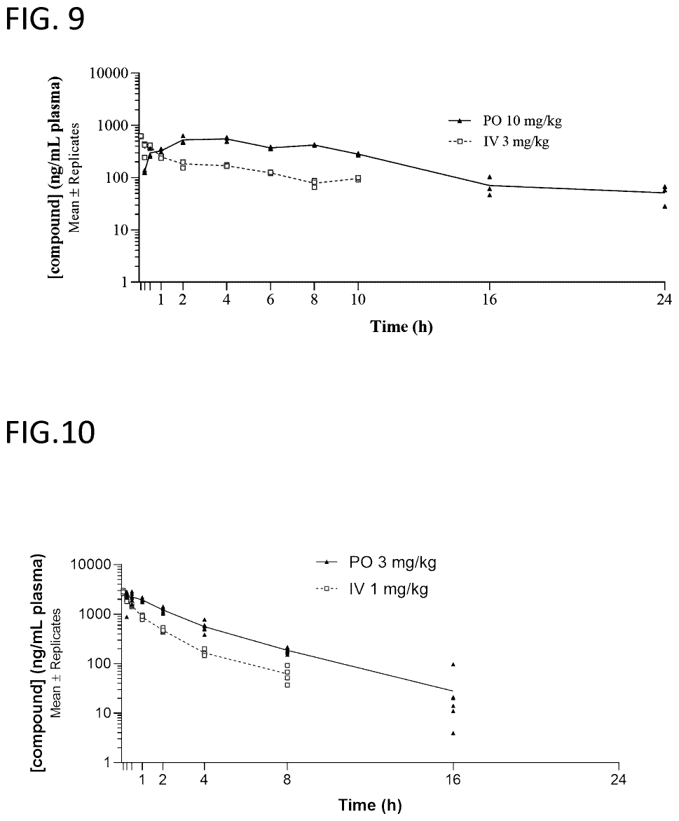

FIG. 9 shows Plasma concentration across time following administration of compound 69 as a single bolus via IV and PO gavage to mice. n=3/timepoint. Timepoints include 5 min (IV only), 15 min, 30 min, 1 h, 2 h, 4 h, 6 h, 8 h, 10 h, 16 h (PO only), 24 h (PO only).

TABLE J
Pharmacokinetic parameters resulting from non-compartmental
analysis (NCA) of Compound 69 administered to Rat

| Compound | | Compound 69 | | | |
|---|---|---|---|---|---|
| Sample Type | | Rat Plasma | Rat Plasma | Rat Plasma | Rat Plasma |
| Route of Admin. | | IV | PO | PO | PO |
| Vehicle | | 8% w/v HPbCD, | 0.5% w/v HPMC 0.2% w/v Tween80, acetate buffer | | |
| | | 0.7% w/v sodium | (10 mM, pH 4) | | |
| | | chloride, acetate | | | |
| | | buffer (10 mM, | | | |
| | | pH 4) | | | |
| Dose | (mg/kg) | 3 | 3 | 10 | 30 |
| Cₘₐₓ (5 min) | (ng/mL) | 183.04 | 27.18 | 140.29 | 482.33 |
| T_{1/2} | (h) | 4.82 | 2.81 | 3.23 | 6.47 |
| AUCₗₐₛₜ | (h*ng/mL) | 487.50 | 304.55 | 1550.36 | 5867.04 |
| AUC_{INF}_{_{—}}_{obs} | (h*ng/mL) | 500.56 | 307.85 | 1568.60 | 5888.67 |
| AUC_{_{—}}_{% Extrap}_{_{—}}_{obs} | (%) | 2.61 | 1.07 | 1.16 | 0.37 |
| Vz_obs | (mL/kg) | 41648.41 | 39567.80 | 29721.18 | 47527.84 |
| Cl_obs | (mL/h/kg) | 5993.23 | 9745.00 | 6375.12 | 5094.53 |
| Rsq | | 0.99 | 0.95 | 0.93 | 1.00 |
| F | % | — | 62% | 95% | 120% |

TABLE K
Pharmacokinetic parameters resulting from non-compartmental
analysis (NCA) of Compound 69 administered to Dog

| Compound | | Compound 69 | |
|---|---|---|---|
| Sample Type | | Dog Plasma | Dog Plasma |
| Route of Admin. | | IV | PO |
| Vehicle | | 15% w/v HPbCD, 0.5% w/v | 0.5% w/v HPMC 0.2% w/v |
| | | NaCl, acetate buffer | Tween80, acetate buffer |
| | | (10 mM, pH 5.5) | (10 mM, pH 4) |
| Dose | (mg/kg) | 3 | 10 |
| Cₘₐₓ (5 min) | (ng/mL) | 397.23 | 291.34 |
| T_{1/2} | (h) | 13.28 | 13.76 |
| AUCₗₐₛₜ | (h*ng/mL) | 1184.05 | 5635.35 |
| AUC_{INF}_{_{—}}_{obs} | (h*ng/mL) | 1555.26 | 6217.47 |
| AUC_{_{—}}_{% Extrap}_{_{—}}_{obs} | (%) | 23.94 | 9.05 |
| Vz_obs | (mL/kg) | 37411.97 | 32693.79 |
| Cl_obs | (mL/h/kg) | 1947.37 | 1659.86 |
| Rsq | | 0.98 | 0.99 |
| F | % | — | 143% |

TABLE L
Pharmacokinetic parameters resulting from non-compartmental
analysis (NCA) of Compound 54 administered to Mice

| Compound | | Compound 54 | |
|---|---|---|---|
| Sample Type | | Mouse Plasma | Mouse Plasma |
| Route of Admin. | | IV | PC |
| Vehicle | | 3.2% (w/v) HPBCD in 135 mM | 5% (w/v) HPBCD in 100 mM |
| | | Saline and 10 mM acetate | Saline and 10 mM acetate |
| | | buffer pH 5.0 | buffer pH 5.0 |
| Dose | (mg/kg) | 1 | 3 |
| Cₘₐₓ (5 min) | (ng/mL) | 2824 (±92) | 2221 (±191) |
| C₀ | (ng/mL) | 3314 | — |
| T_{1/2} | (h) | 1.69 | 2.33 |
| AUCₗₐₛₜ | (h*ng/mL) | 3704 (±95) | 7653 (±253) |
| AUC_{INF}_{_{—}}_{obs} | (h*ng/mL) | 3707 | 7658 |
| AUC_{_{—}}_{% Extrap}_{_{—}}_{obs} | (%) | 0.10 | 0.07 |
| Vz_obs | (mL/kg) | 646 | 1205^{a} |
| Cl_obs | (mL/h/kg) | 264 | 358^{b} |
| Rsq | | 0.990 | 0.993 |
| F | % | — | 74% |

FIG. 10 shows Plasma concentration across time following administration of compound 54 as a single bolus via IV and PO gavage to mice. n=4/timepoint for IV, n=6/timepoint PO. Timepoints include 5 min (IV only), 15 min, 30 min, 1 h, 2 h, 4 h, 8 h, 16 h, 24 h (PO only).

### Example 15: Activity in In Vivo Biological Assays

### Assay 1: Clonogenic Survival

FaDu tumour xenograft-bearing Rag2M mice were treated with single administrations of test compounds at indicated doses±radiation as indicated, at time 0.

Mice were irradiated at 1 h post compound administration (unless otherwise indicated), with 300 KV X-rays and a 10 mA current from a Precision XRAD 300 machine (USA) with a beam-hardening filter (2 mm Al+0.25 mm Cu+0.75 mm Sn) and an adjustable site to specimen distance (SSD) at a dose of 8-10 Gy, delivered at a rate of ˜1.05 Gy/min.

Mice were terminated at times after irradiation as indicated, and tumour tissues excised. A portion of tumour approximately 300-500 mg was briefly minced using sterile scissors and placed in a pre-weighed gentleMACS C tube (Miltenyi Biotec cat #130-093-237) and weighed with combined tumour+tube mass recorded. A 5 mL aliquot of enzyme cocktail comprising of trypsin/DNase/collagenase/EDTA-Na₂ in PBS (1 mg/ml trypsin+0.26 mg/ml DNase+0.21 mg/ml collagenase+2 mM EDTA-Na2) was added to the tube, which is then run on the gentleMACS Dissociator disaggregation system. Tubes were then incubated at 37° C. and continually rotated for 20 min before again running on the gentleMACS Dissociator program. Samples were then diluted with 5 ml MEM medium containing 5% FBS and filtered (Falcon cell strainer 100 μm, cat #352360). After centrifugation (1100 rpm for 6 minutes at 4° C.) supernatant was aspirated and the cells resuspended in 4 ml cold growth medium (MEM+10% FBS)+50 ul DNase (5 mg/ml DNase solution). Cell numbers were counted from the same cell solution using a hemacytometer. Appropriate dilutions (control: 10,000 cells/ml; 8Gy: 400,000 cells/ml; drug+radiation treated; 500,000 cells/ml) were made and cells then plated in triplicate: control: 100 μl and 300 μl (1000 and 3000 cells/6 cm tissue culture plate); 8Gy: 30 μl, 105 μl and 350 μl (12000, 42000 cells/6 cm plates and 140000 cells/10 cm plate); drug+8Gy: 70 μl, 300p, 1.2 ml (35000 and 150000 cells/6 cm plates and 600000 cells/10 cm plates—only duplicate). Plates were incubated at 37° C. (in a 5% CO2, 5% O2 and balance N2 environment) for 14 days. For a final and accurate automated live-cell counting a solution of Hoechst33342 (0.01 mg/ml)+Propidium Iodide (0.004 mg/ml) in 0.9% saline was mixed with an appropriate cell number dilution of the samples in 96 well plates (50 ul staining solution+50 ul cell suspension of 10000 cells/ml) and images are taken of the wells and cell numbers are calculated by a computer program.

After 14 days plates were removed from the incubator, rinsed with PBS and stained with cold 2 g/L Malachite green oxalate salt solution (Sigma-Aldrich, cat #M6880) for 30 minutes at room temperature. Plates were rinsed with distilled water and dried. Pictures were obtained of the plates and colonies consisting of 50 or more cells were counted using a Stuart Scientific colony counter.

All plates with colonies were analyzed except when colony numbers were so high that the edges of individual colonies could not be determined accurately. Plating efficiency (PE) of each tumour sample was calculated first: PE is a ratio of the number of colonies to the number of cells seeded (sum of all the colonies countable in all the plates of the same sample divided by the sum of cells plated in the same plates). The PE of control tumours was averaged for the calculation of surviving fraction (SF): SF is based on the number of colonies formed in drug and/or radiation treated cells relative to that of untreated control (PE of treated tumor sample divided by PE of control). All treated data points were compared separately to the average of the controls. Finally a Clonogenic Survival Enhancement Ratio (CS-ER) was calculated: the SF of irradiated control groups were divided by the SF of treatment groups to determine treatment group-specific CS-ER values, and these were assigned scores based on defined response ranges: values>6=A, 4-6=B, <4=C.

| | | in vivo | |
|---|---|---|---|
| | | clonogenic | CS-ER |
| | Compound | survival | Range |
| Structure | # | dose | Score |
| 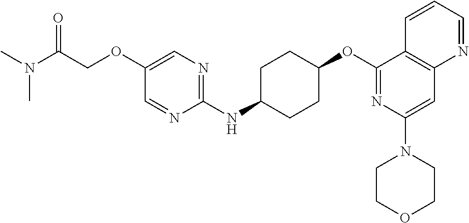 [SMILES: CN(C)C(=O)COc1cnc(N[C@H]2CC[C@@H](Oc3nc(N4CCOCC4)cc4ncccc34)CC2)nc1] | 54 | 100 mpk + 10 Gy, 0.5 h 100 mpk + 10 Gy, 4 h 100 mpk + 10 Gy, 8 h 100 mpk + 10 Gy, 24 h | B A B A |
| 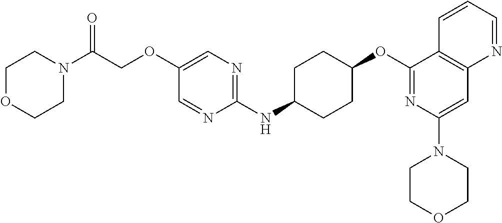 [SMILES: O=C(COc1cnc(N[C@H]2CC[C@@H](Oc3nc(N4CCOCC4)cc4ncccc34)CC2)nc1)N1CCOCC1] | 61 | 30 mpk + 8 Gy, 4 h 30 mpk + 8 Gy, 2 h | B C |
| 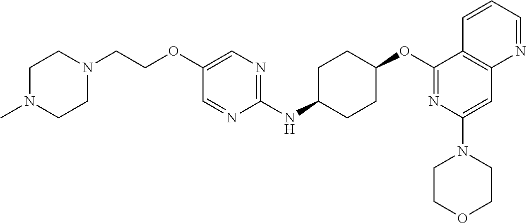 [SMILES: CN1CCN(CCOc2cnc(N[C@H]3CC[C@@H](Oc4nc(N5CCOCC5)cc5ncccc45)CC3)nc2)CC1] | 69 | 30 mpk + 8 Gy, 4 h | C |
| 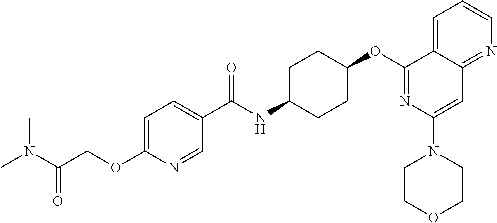 [SMILES: CN(C)C(=O)COc1ccc(C(=O)N[C@H]2CC[C@@H](Oc3nc(N4CCOCC4)cc4ncccc34)CC2)cn1] | 117 | 30 mpk + 8 Gy, 4 h 30 mpk + 8 Gy, 2 h | B C |
| 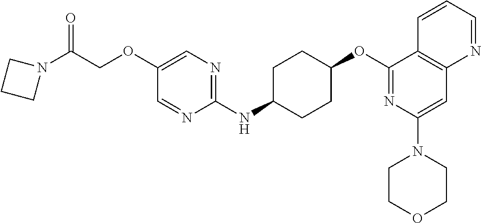 [SMILES: O=C(COc1cnc(N[C@H]2CC[C@@H](Oc3nc(N4CCOCC4)cc4ncccc34)CC2)nc1)N1CCC1] | 75 | 30 mpk + 8 Gy, 4 h 30 mpk + 8 Gy, 2 h | B B |
| 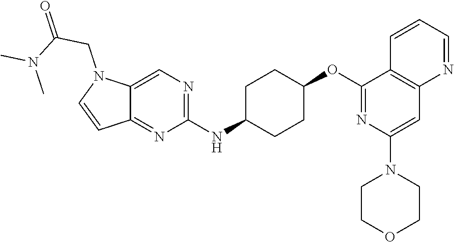 [SMILES: CN(C)C(=O)Cn1ccc2nc(N[C@H]3CC[C@@H](Oc4nc(N5CCOCC5)cc5ncccc45)CC3)ncc21] | 86 | 30 mpk + 8 Gy, 4 h 30 mpk + 8 Gy, 2 h | B B |
| 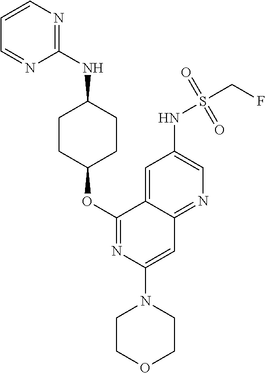 [SMILES: O=S(=O)(CF)Nc1cnc2cc(N3CCOCC3)nc(O[C@H]3CC[C@@H](Nc4ncccn4)CC3)c2c1] | 192 | 30 mpk + 8 Gy, 4 h 30 mpk + 8 Gy, 2 h | B A |
| 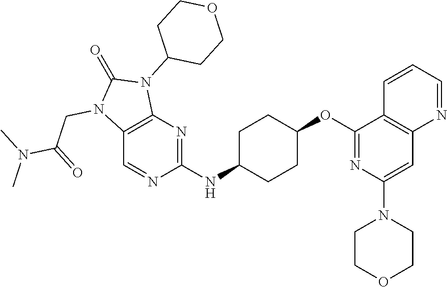 [SMILES: CN(C)C(=O)Cn1c(=O)n(C2CCOCC2)c2nc(N[C@H]3CC[C@@H](Oc4nc(N5CCOCC5)cc5ncccc45)CC3)ncc21] | 97 | 30 mpk + 8 Gy, 4 h 30 mpk + 8 Gy, 2 h | B C |

### Assay 2: Tumour Growth Delay

FaDu tumour xenograft-bearing animals were monitored for tumour growth and once average tumour size reached approximately 100-250 mm3, animals were assigned to treatment cohorts using stratified randomization. Animals were weighted and treated with doses of test compounds at 10, 30 or 100 mg/kg PO from 1, 3 or 10 mg/mL formulations at time 0.

One hour following the first injection, animals received irradiation to the tumour site with 300 KV X-rays and a 10 mA current from a Precision XRAD 300 machine (USA) with a beam-hardening filter (2 mm Al+0.25 mm Cu+0.75 mm Sn) and an adjustable site to specimen distance (SSD) at a dose of 5-10 Gy, as indicated, delivered at a rate of ˜1.05 Gy/min. For BID dosing regimens, at 8 h following the first injection, animals were again administered with test compound at the same dose as their previous administration. Animals were weighed and had tumours measured using calipers three times weekly until the endpoint of maximum tumour volume, 1000 mm3, was reached and animals were euthanized. Tumour volumes were calculated according to the equation L×W²/2 with the length (mm) being the longer axis of the tumour; in the case that no tumour was palpable, tumour volumes were recorded as 0 mm³.

Data were calculated as tumour growth normalized to day 0 measurements, with time to tumour doubling determined. Average time to tumour doubling for each group was used to determine a Tumour Growth Delay Enhancement Ratio (TGD-ER): the time to doubling for treatment groups were divided by the time to doubling for irradiation-alone groups to determine group-specific TGD-ER values, and these were assigned scores based on defined response ranges: values>2.5=A, 1-2.5=B, <1=C.

| | | in vivo | TGD-ER |
|---|---|---|---|
| | | growth | Range |
| Structure | Compound # | delay dose | Score |
| 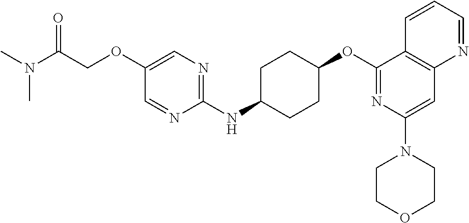 [SMILES: CN(C)C(=O)COc1cnc(N[C@H]2CC[C@@H](Oc3nc(N4CCOCC4)cc4ncccc34)CC2)nc1] | 54 | 10 mpk + 10 Gy 30 mpk + 10 Gy 100 mpk + 10 Gy | B A B |
| 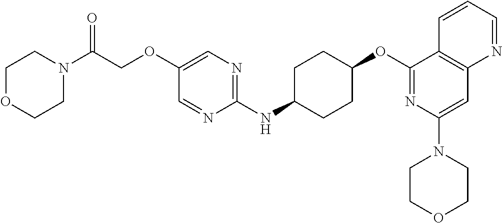 [SMILES: O=C(COc1cnc(N[C@H]2CC[C@@H](Oc3nc(N4CCOCC4)cc4ncccc34)CC2)nc1)N1CCOCC1] | 61 | (30 mpk) Q5D (30 mpk + 2 Gy) Q5D | C A |
| 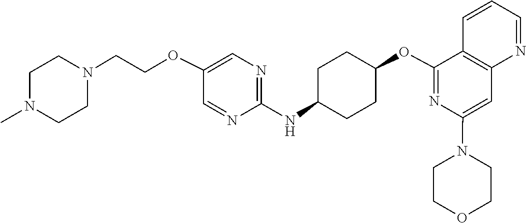 [SMILES: CN1CCN(CCOc2cnc(N[C@H]3CC[C@@H](Oc4nc(N5CCOCC5)cc5ncccc45)CC3)nc2)CC1] | 69 | 30 mpk BID + 10 Gy 30 mpk + @4 h 10 Gy 100 mpk + @4 h 10 Gy (30 mpk) Q5D (30 mpk + 2 Gy) Q5D | B C B C A |
| 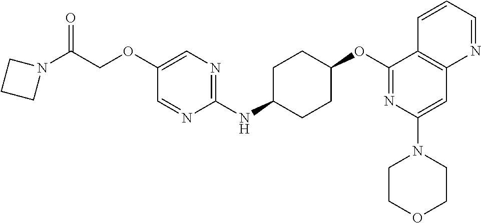 [SMILES: O=C(COc1cnc(N[C@H]2CC[C@@H](Oc3nc(N4CCOCC4)cc4ncccc34)CC2)nc1)N1CCC1] | 75 | 30 mpk BID + 10 Gy | B |
| 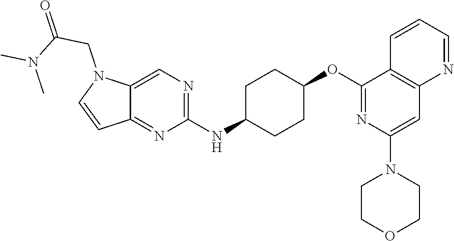 [SMILES: CN(C)C(=O)Cn1ccc2nc(N[C@H]3CC[C@@H](Oc4nc(N5CCOCC5)cc5ncccc45)CC3)ncc21] | 86 | (30 mpk) Q5D (30 mpk + 2 Gy) Q5D 30 mpk BID + 10 Gy | B A B |
| 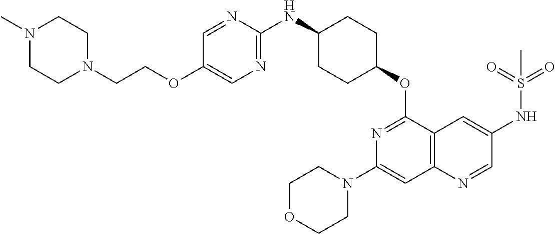 [SMILES: CN1CCN(CCOc2cnc(N[C@H]3CC[C@@H](Oc4nc(N5CCOCC5)cc5ncc(NS(C)(=O)=O)cc45)CC3)nc2)CC1] | LFA031 | 30 mpk BID + 10 Gy | B |
| 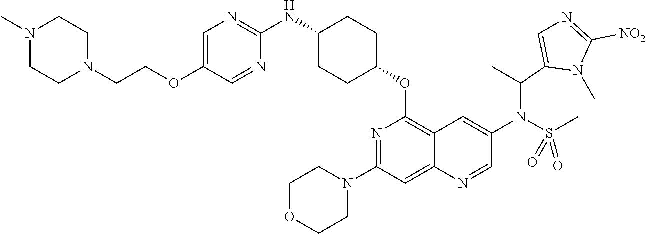 [SMILES: CC(c1cnc([N+](=O)[O-])n1C)N(c1cnc2cc(N3CCOCC3)nc(O[C@H]3CC[C@@H](Nc4ncc(OCCN5CCN(C)CC5)cn4)CC3)c2c1)S(C)(=O)=O] | LFA033 | 50 mpk BID + 10 Gy | C |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A compound of formula (II): 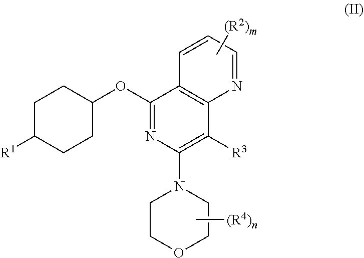
wherein:
R¹ is selected from 5- to 10-membered heteroaryl, NR⁶R⁷, C(O)R⁷, C(O)NR⁶R⁷, N═C(NR⁶R⁷)₂, wherein each heteroaryl is optionally substituted with from 1 to 5 R⁸ substituents;
each R² is independently selected from C₂-C₆-alkynyl, halo, OR⁵, NR⁶R⁷, COOR⁶, C(O)NR⁶R⁷, C₃-C₈-cycloalkyl, 3- to 8-membered heterocycloalkyl, 5- to 10-membered aryl, 5- to 10-membered heteroaryl, S—C₁-C₆-alkyl, S(O)₂—C₁-C₆-alkyl, and S(O)₂—NR¹⁰R¹⁰, wherein each alkyl, alkynyl, cycloalkyl, heterocycloalkyl, aryl, heteroaryl is optionally substituted with from 1 to 5 R⁹ substituents;
R³ is selected from H, halo, C₁-C₆-alkyl and C₁-C₆-haloalkyl;
each R⁴ is independently selected from halo, C₁-C₆-alkyl and C₁-C₆-haloalkyl, wherein two R⁴ groups are optionally linked to form a 5- to 7-membered heterocycloalkyl;
each R⁵ is independently selected from H and C₁-C₆-alkyl, wherein each alkyl is optionally substituted with from 1 to 5 R⁹ substituents;
each R⁶ is independently selected from H, C₁-C₆-alkyl and COOR⁵;
each R⁷ is independently selected from H, OR⁵, cyano, C₁-C₆-alkyl, C₃-C₈-cycloalkyl, 3- to 10-membered heterocycloalkyl, 5- to 10-membered aryl, 5- to 10-membered heteroaryl, C(O)—C₁-C₆-alkyl, C(O)—(C₃-C₈-cycloalkyl), C(O)-(3- to 8-membered heterocycloalkyl), C(O)-(5- to 10-membered aryl), C(O)-(5- to 10-membered heteroaryl), C(O)—O—C₁-C₆-alkyl, S(O)₂—C₁-C₆-alkyl, S(O)₂—(C₃-C₈-cycloalkyl), S(O)₂-(3- to 8-membered heterocycloalkyl), S(O)₂-(5- to 10-membered aryl), S(O)₂-(5- to 10-membered heteroaryl), C(O)NR¹⁰R¹⁰, C(═NH)NH₂, and S(O)₂—NR¹⁰R¹⁰, wherein each alkyl, cycloalkyl, heterocycloalkyl, aryl and heteroaryl is optionally substituted with from 1 to 5 R¹¹ substituents;
each R⁸ is independently selected from C₁-C₆-alkyl, C₁-C₆-haloalkyl, and halo;
each R⁹ is independently selected from OH, oxo, C₁-C₆-alkyl, 3- to 8-membered heterocycloalkyl, and 5- to 10-membered aryl, wherein each alkyl, heterocycloalkyl and aryl is optionally substituted with from 1 to 5 R¹¹ substituents;
each R¹⁰ is independently selected from H, C₁-C₆-alkyl, C₁-C₆-alkoxy, 5- to 10-membered aryl, and S(O)₂—C₁-C₆-alkyl, wherein each alkyl and aryl is optionally substituted with from 1 to 5 R¹¹ substituents;
each R¹¹ is independently selected from OH, oxo, halo, cyano, C₁-C₆-alkyl, C₁-C₆-haloalkyl, C₁-C₆-alkoxy, C₃-C₈-cycloalkyl, 3- to 8-membered heterocycloalkyl, 5- to 10-membered aryl, 5- to 10-membered heteroaryl, NR⁶R⁷, C(O)NR⁶R⁷, C(O)OR⁷, and S(O)₂NR⁶R⁷, wherein each alkyl, haloalkyl, alkoxy, cycloalkyl, heterocycloalkyl, aryl and heteroaryl is optionally substituted with 1 to 5 R¹² substituents;
each R¹² is independently selected from halo, OH, oxo, cyano, C₁-C₆-alkyl, C₁-C₆-haloalkyl, C₁-C₆-alkoxy, 3- to 8-membered heterocycloalkyl, 5- to 10-membered heteroaryl, nitro, and NR¹⁰R¹⁰, wherein each alkyl, haloalkyl, alkoxy, heterocycloalkyl and heteroaryl is optionally substituted with 1 to 5 R¹³ substituents;
each R¹³ is independently selected from OH, halo, C₁-C₆-alkyl, and C₁-C₆-haloalkyl;
m is 0 or an integer selected from 1, 2 and 3; and
n is 0 or an integer selected from 1, 2, 3 and 4,
or a prodrug or a pharmaceutically acceptable salt thereof.

2. The compound of claim 1, wherein m is 0.

3. The compound of claim 1, wherein n is 0.

4. The compound of claim 1, wherein R¹ is C(O)NR⁶R⁷.

5. The compound of claim 1, wherein R¹ is N═C(NR⁶R⁷)₂.

6. The compound of claim 1, wherein R² is C₂-C₆-alkynyl.

7. The compound of claim 1, wherein R² is COOR⁶.

8. The compound of claim 1, wherein R² is C(O)NR⁶R⁷.

9. The compound of claim 1, wherein R² is 5- to 10-membered aryl or 5- to 10-membered heteroaryl.

10. The compound of claim 1, wherein R² is 3- to 8-membered heterocycloalkyl.

11. The compound of claim 1, wherein R² is S—C₁-C₆-alkyl or S(O)₂—C₁-C₆-alkyl.

12. The compound of claim 1, wherein R³ is H.

13. The compound of claim 1, wherein R⁴ is halo.

14. The compound of claim 1, wherein n is 2, and two R⁴ groups are linked to form a 5- to 7-membered heterocycloalkyl.

15. The compound of claim 1, wherein the compound is of formula (IIa): 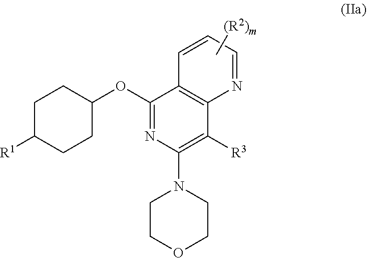
wherein:
R¹ is NR⁶R⁷;
R⁶ is H;
R⁷ is selected from C(O)—C₁-C₆-alkyl, C(O)—(C₃-C₈-cycloalkyl), C(O)-(3- to 8-membered heterocycloalkyl), C(O)-(5- to 10-membered aryl), C(O)-(5- to 10-membered heteroaryl), C(O)NR¹⁰R¹⁰, and C(═NH)NH₂, wherein each alkyl, cycloalkyl, heterocycloalkyl, aryl and heteroaryl is optionally substituted with from 1 to 5 R¹¹ substituents.

16. The compound of claim 1, wherein the compound is of formula (IIb): 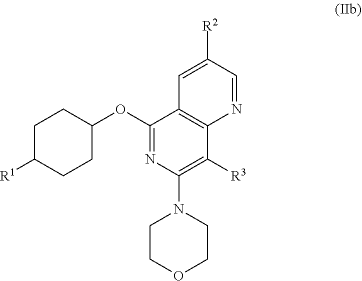
wherein:
R² is NR⁶R⁷;
R⁶ is H;
R⁷ is selected from C(O)NR¹⁰R¹⁰, S(O)₂—C₁-C₆-alkyl, S(O)₂—(C₃-C₈-cycloalkyl), S(O)₂-(3- to 8-membered heterocycloalkyl), S(O)₂-(5- to 10-membered aryl), S(O)₂-(5- to 10-membered heteroaryl), and S(O)₂—NR¹⁰R¹⁰, wherein each alkyl, cycloalkyl, heterocycloalkyl, aryl and heteroaryl is optionally substituted with from 1 to 5 R¹¹ substituents.

17. The compound of claim 1, wherein the compound is of formula (IIc): 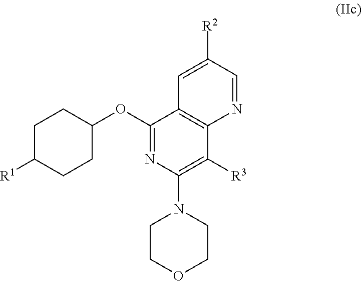
wherein:
R² is OR⁵;
R⁵ is C₁-C₆-alkyl substituted with from 1 to 5 R⁹ substituents.

18. The compound of claim 1, wherein the compound is of formula (IId): 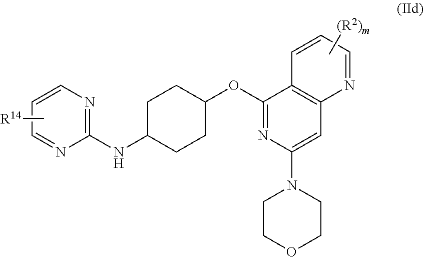
wherein:
R¹⁴ is C₁-C₆-alkoxy, wherein the alkoxy is substituted with 1 to 2 R¹⁵ substituents,
wherein when the alkoxy of R¹⁴ is substituted with one R¹⁵ substituent, R¹⁵ is selected from 3- to 8-membered heterocycloalkyl, 5- to 10-membered heteroaryl, and NR¹⁷R¹⁷, wherein each heterocycloalkyl and heteroaryl is optionally substituted with 1 to 2 R¹⁶ substituents;
wherein when the alkoxy of R¹⁴ is substituted with two R¹⁵ substituents, each R¹⁵ is independently selected from OH, oxo, 3- to 8-membered heterocycloalkyl, and NR¹⁷R¹⁷, wherein heterocycloalkyl is optionally substituted with 1 to 2 R¹⁶ substituents;
each R¹⁶ is independently selected from OH, halo, and C₁-C₆-alkyl;
each R¹⁷ is independently selected from H, C₁-C₆-alkyl, C₁-C₆-alkoxy, 5- to 10-membered aryl, and S(O)₂—C₁-C₆-alkyl, wherein each alkyl and aryl is optionally substituted with from 1 to 2 R¹⁸ substituents;
each R¹⁸ is independently selected from OH, oxo, halo, cyano, C₁-C₆-alkyl, C₁-C₆-haloalkyl, C₁-C₆-alkoxy, C₃-C₈-cycloalkyl, 3- to 8-membered heterocycloalkyl, 5- to 10-membered aryl, 5- to 10-membered heteroaryl, NR¹⁹R²⁰, C(O)NR¹⁹R²⁰, C(O)OR²⁰, and S(O)₂NR¹⁹R²⁰; and
each of R¹⁹ and R²⁰ is independently selected from H and C₁-C₆-alkyl.

19. The compound of claim 1, wherein:
R¹ is NR⁶R⁷;
R⁶ is H;
R⁷ is 5- to 10-membered heteroaryl, wherein heteroaryl is substituted with C₁-C₆-alkyl, wherein alkyl is substituted with oxo and NR²¹R²², wherein each of R²¹ and R²² is independently selected from H and C₁-C₆-alkyl.

20. The compound of claim 1, wherein:
R¹ is NR⁶R⁷;
R⁶ is H;
R⁷ is 5- to 10-membered heteroaryl, wherein heteroaryl is substituted with from 2 to 3 R¹¹ substituents;
wherein one R¹¹ substituent is oxo, and each additional R¹¹ substituent is independently selected from OH, halo, cyano, C₁-C₆-alkyl, C₁-C₆-haloalkyl, C₁-C₆-alkoxy, C₃-C₈-cycloalkyl, 3- to 8-membered heterocycloalkyl, 5- to 10-membered aryl, 5- to 10-membered heteroaryl, NR²³R²⁴, C(O)NR²³R²⁴, C(O)OR²⁴, and S(O)₂NR²³R²⁴, wherein each alkyl, haloalkyl, alkoxy, cycloalkyl, heterocycloalkyl, aryl and heteroaryl is optionally substituted with 1 to 5 R¹² substituents;
each of R²³ and R²⁴ is independently selected from H and C₁-C₆-alkyl.

21. The compound of claim 1, wherein the compound is selected from:

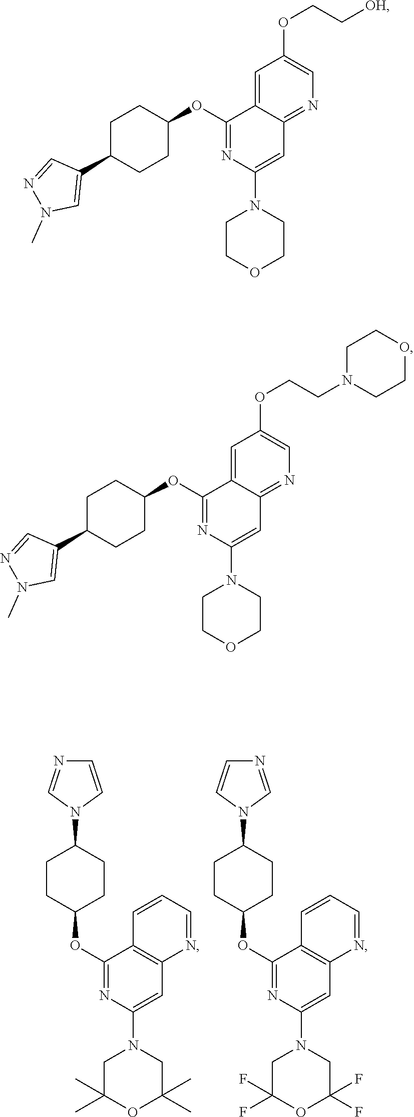

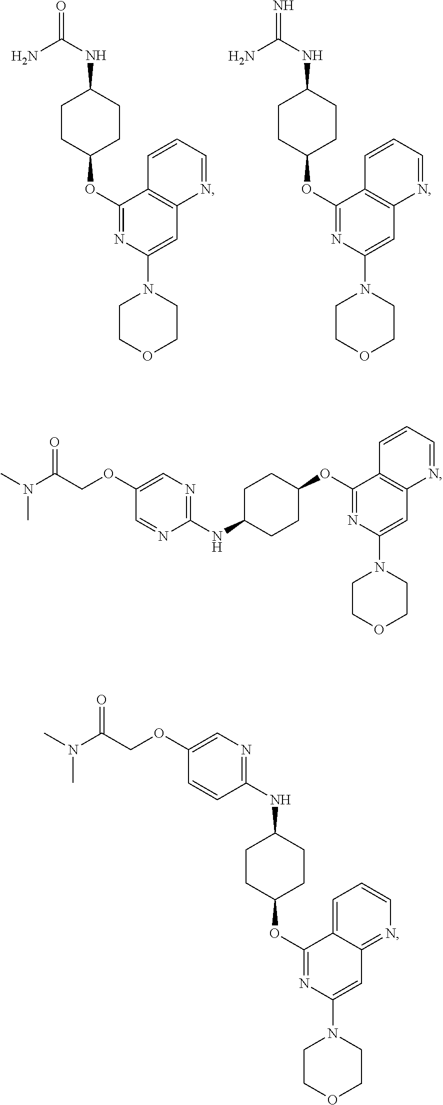 [SMILES: NC(=O)N[C@H]1CC[C@@H](Oc2nc(N3CCOCC3)cc3ncccc23)CC1 ; N=C(N)N[C@H]1CC[C@@H](Oc2nc(N3CCOCC3)cc3ncccc23)CC1 ; CN(C)C(=O)COc1cnc(N[C@H]2CC[C@@H](Oc3nc(N4CCOCC4)cc4ncccc34)CC2)nc1 ; CN(C)C(=O)COc1ccc(N[C@H]2CC[C@@H](Oc3nc(N4CCOCC4)cc4ncccc34)CC2)nc1]

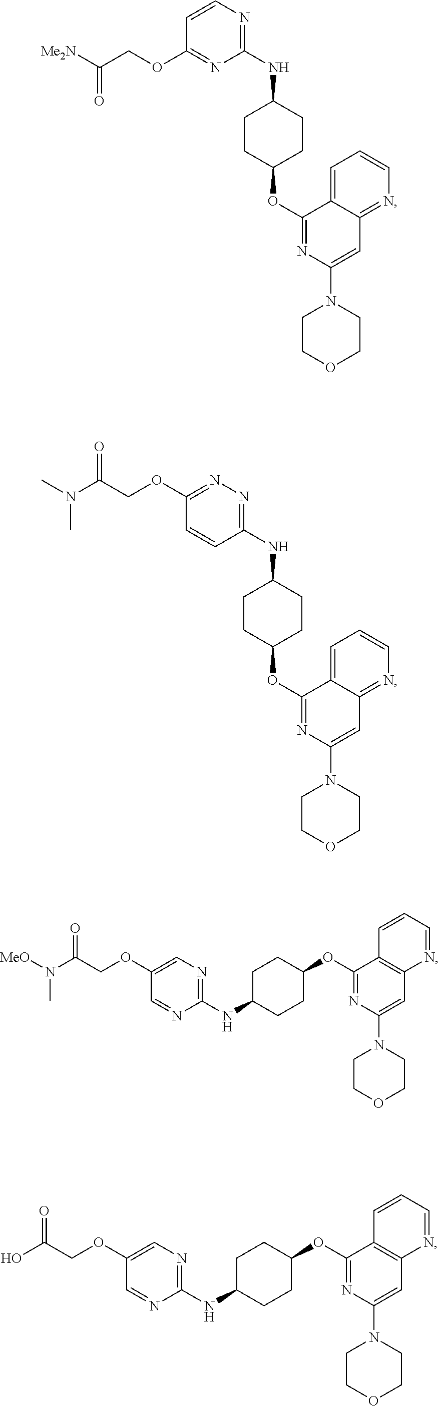 [SMILES: CN(C)C(=O)COc1ccnc(N[C@H]2CC[C@@H](Oc3nc(N4CCOCC4)cc4ncccc34)CC2)n1 ; CN(C)C(=O)COc1ccc(N[C@H]2CC[C@@H](Oc3nc(N4CCOCC4)cc4ncccc34)CC2)nn1 ; CON(C)C(=O)COc1cnc(N[C@H]2CC[C@@H](Oc3nc(N4CCOCC4)cc4ncccc34)CC2)nc1 ; O=C(O)COc1cnc(N[C@H]2CC[C@@H](Oc3nc(N4CCOCC4)cc4ncccc34)CC2)nc1]

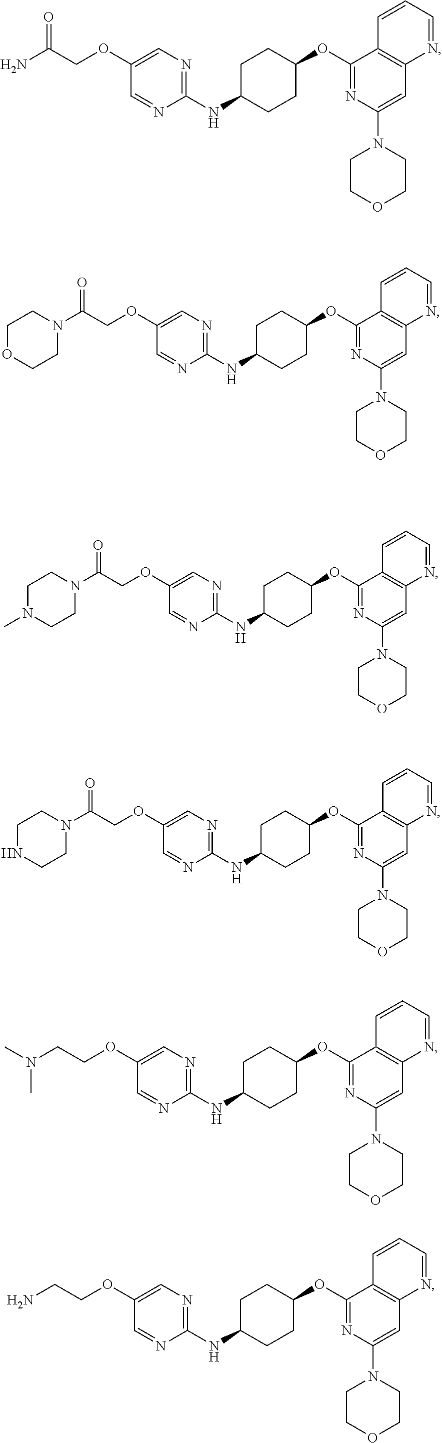 [SMILES: NC(=O)COc1cnc(N[C@H]2CC[C@@H](Oc3nc(N4CCOCC4)cc4ncccc34)CC2)nc1 ; O=C(COc1cnc(N[C@H]2CC[C@@H](Oc3nc(N4CCOCC4)cc4ncccc34)CC2)nc1)N1CCOCC1 ; CN1CCN(C(=O)COc2cnc(N[C@H]3CC[C@@H](Oc4nc(N5CCOCC5)cc5ncccc45)CC3)nc2)CC1 ; O=C(COc1cnc(N[C@H]2CC[C@@H](Oc3nc(N4CCOCC4)cc4ncccc34)CC2)nc1)N1CCNCC1 ; CN(C)CCOc1cnc(N[C@H]2CC[C@@H](Oc3nc(N4CCOCC4)cc4ncccc34)CC2)nc1 ; NCCOc1cnc(N[C@H]2CC[C@@H](Oc3nc(N4CCOCC4)cc4ncccc34)CC2)nc1]

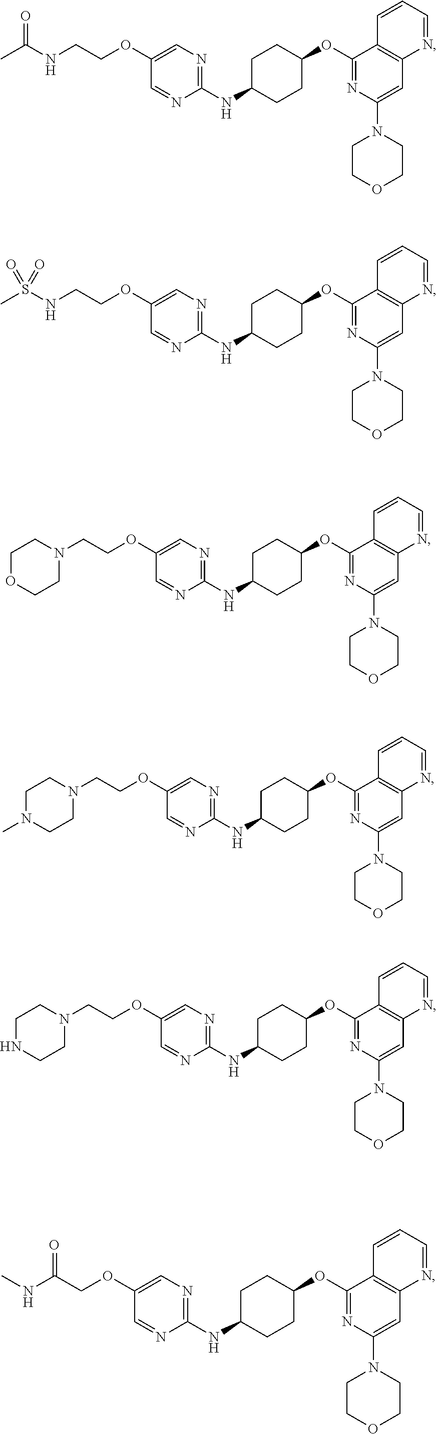 [SMILES: CC(=O)NCCOc1cnc(N[C@H]2CC[C@@H](Oc3nc(N4CCOCC4)cc4ncccc34)CC2)nc1 ; CS(=O)(=O)NCCOc1cnc(N[C@H]2CC[C@@H](Oc3nc(N4CCOCC4)cc4ncccc34)CC2)nc1 ; c1cnc2cc(N3CCOCC3)nc(O[C@H]3CC[C@@H](Nc4ncc(OCCN5CCOCC5)cn4)CC3)c2c1 ; CN1CCN(CCOc2cnc(N[C@H]3CC[C@@H](Oc4nc(N5CCOCC5)cc5ncccc45)CC3)nc2)CC1 ; c1cnc2cc(N3CCOCC3)nc(O[C@H]3CC[C@@H](Nc4ncc(OCCN5CCNCC5)cn4)CC3)c2c1 ; CNC(=O)COc1cnc(N[C@H]2CC[C@@H](Oc3nc(N4CCOCC4)cc4ncccc34)CC2)nc1]

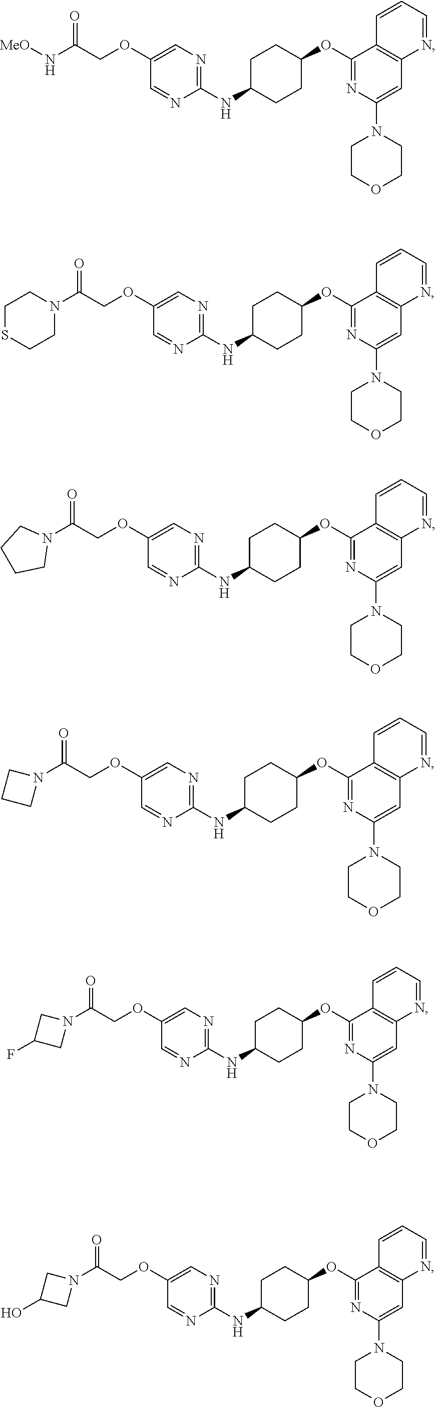 [SMILES: CONC(=O)COc1cnc(N[C@H]2CC[C@@H](Oc3nc(N4CCOCC4)cc4ncccc34)CC2)nc1 ; O=C(COc1cnc(N[C@H]2CC[C@@H](Oc3nc(N4CCOCC4)cc4ncccc34)CC2)nc1)N1CCSCC1 ; O=C(COc1cnc(N[C@H]2CC[C@@H](Oc3nc(N4CCOCC4)cc4ncccc34)CC2)nc1)N1CCCC1 ; O=C(COc1cnc(N[C@H]2CC[C@@H](Oc3nc(N4CCOCC4)cc4ncccc34)CC2)nc1)N1CCC1 ; O=C(COc1cnc(N[C@H]2CC[C@@H](Oc3nc(N4CCOCC4)cc4ncccc34)CC2)nc1)N1CC(F)C1 ; O=C(COc1cnc(N[C@H]2CC[C@@H](Oc3nc(N4CCOCC4)cc4ncccc34)CC2)nc1)N1CC(O)C1]

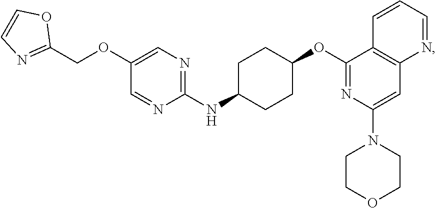 [SMILES: c1cnc2cc(N3CCOCC3)nc(O[C@H]3CC[C@@H](Nc4ncc(OCc5ncco5)cn4)CC3)c2c1]

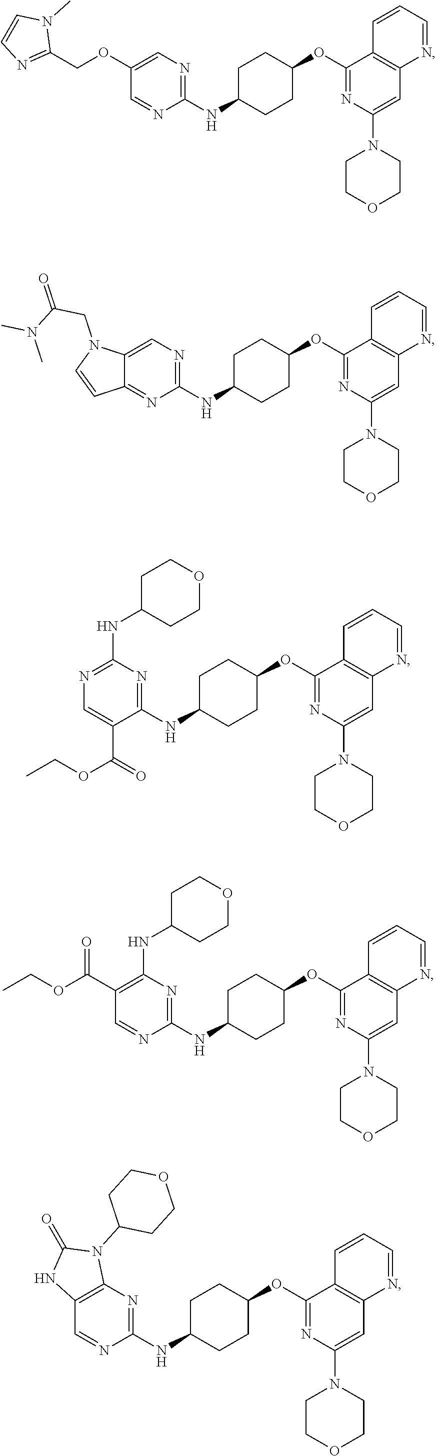 [SMILES: Cn1ccnc1COc1cnc(N[C@H]2CC[C@@H](Oc3nc(N4CCOCC4)cc4ncccc34)CC2)nc1 ; CN(C)C(=O)Cn1ccc2nc(N[C@H]3CC[C@@H](Oc4nc(N5CCOCC5)cc5ncccc45)CC3)ncc21 ; CCOC(=O)c1cnc(NC2CCOCC2)nc1N[C@H]1CC[C@@H](Oc2nc(N3CCOCC3)cc3ncccc23)CC1 ; CCOC(=O)c1cnc(N[C@H]2CC[C@@H](Oc3nc(N4CCOCC4)cc4ncccc34)CC2)nc1NC1CCOCC1 ; O=c1[nH]c2cnc(N[C@H]3CC[C@@H](Oc4nc(N5CCOCC5)cc5ncccc45)CC3)nc2n1C1CCOCC1]

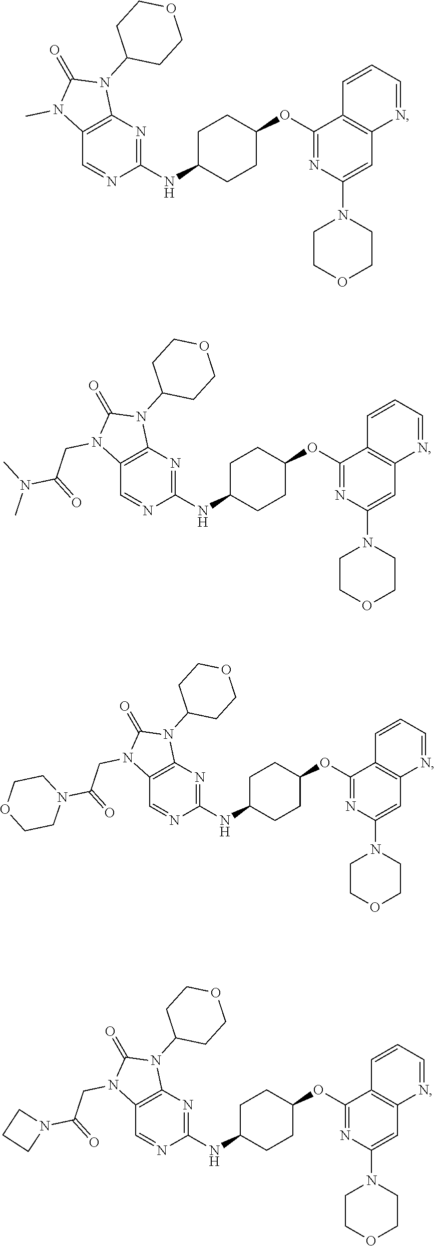 [SMILES: Cn1c(=O)n(C2CCOCC2)c2nc(N[C@H]3CC[C@@H](Oc4nc(N5CCOCC5)cc5ncccc45)CC3)ncc21 ; CN(C)C(=O)Cn1c(=O)n(C2CCOCC2)c2nc(N[C@H]3CC[C@@H](Oc4nc(N5CCOCC5)cc5ncccc45)CC3)ncc21 ; O=C(Cn1c(=O)n(C2CCOCC2)c2nc(N[C@H]3CC[C@@H](Oc4nc(N5CCOCC5)cc5ncccc45)CC3)ncc21)N1CCOCC1 ; O=C(Cn1c(=O)n(C2CCOCC2)c2nc(N[C@H]3CC[C@@H](Oc4nc(N5CCOCC5)cc5ncccc45)CC3)ncc21)N1CCC1]

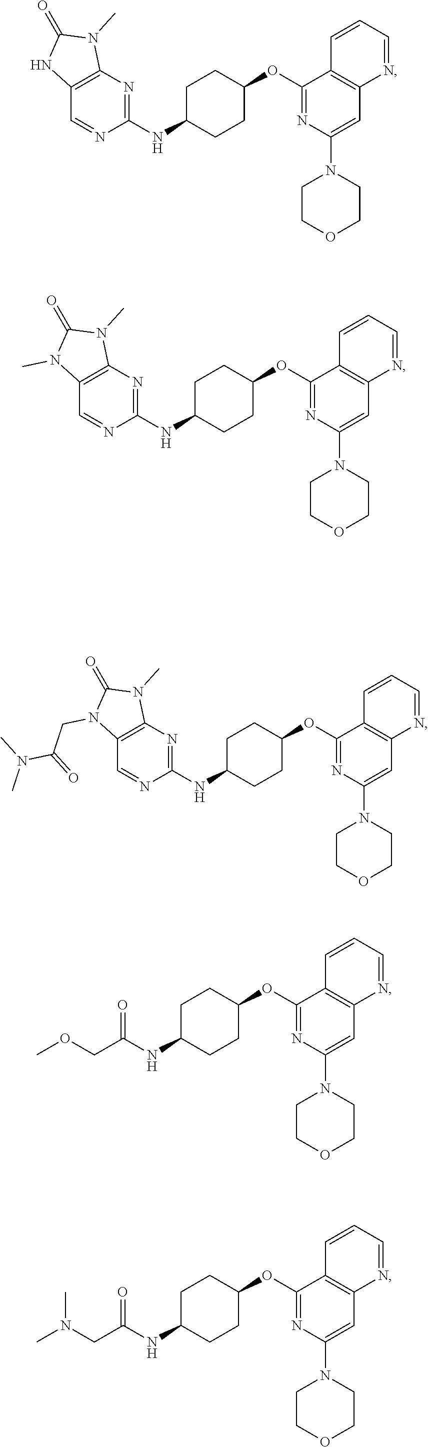 [SMILES: Cn1c(=O)[nH]c2cnc(N[C@H]3CC[C@@H](Oc4nc(N5CCOCC5)cc5ncccc45)CC3)nc21 ; Cn1c(=O)n(C)c2nc(N[C@H]3CC[C@@H](Oc4nc(N5CCOCC5)cc5ncccc45)CC3)ncc21 ; CN(C)C(=O)Cn1c(=O)n(C)c2nc(N[C@H]3CC[C@@H](Oc4nc(N5CCOCC5)cc5ncccc45)CC3)ncc21 ; COCC(=O)N[C@H]1CC[C@@H](Oc2nc(N3CCOCC3)cc3ncccc23)CC1 ; CN(C)CC(=O)N[C@H]1CC[C@@H](Oc2nc(N3CCOCC3)cc3ncccc23)CC1]

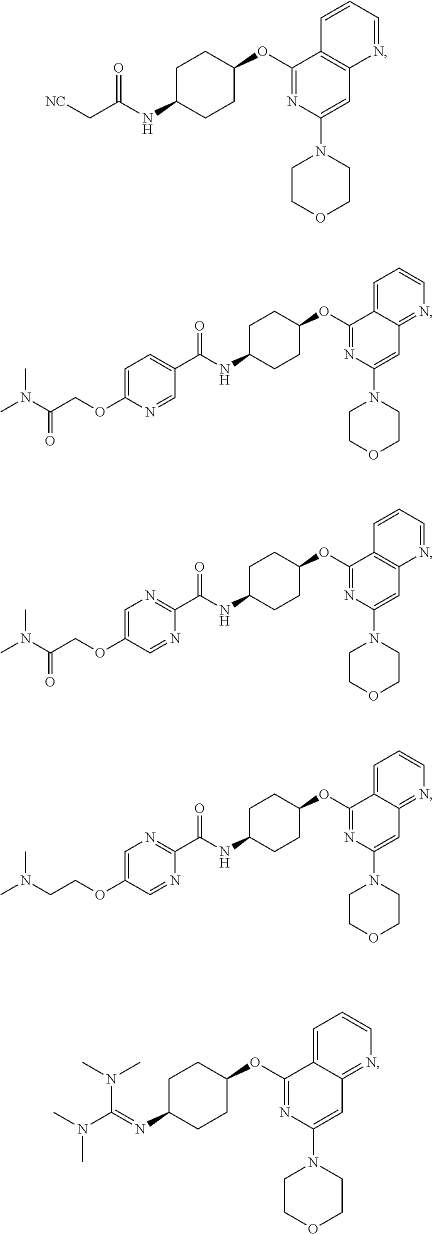 [SMILES: N#CCC(=O)N[C@H]1CC[C@@H](Oc2nc(N3CCOCC3)cc3ncccc23)CC1 ; CN(C)C(=O)COc1ccc(C(=O)N[C@H]2CC[C@@H](Oc3nc(N4CCOCC4)cc4ncccc34)CC2)cn1 ; CN(C)C(=O)COc1cnc(C(=O)N[C@H]2CC[C@@H](Oc3nc(N4CCOCC4)cc4ncccc34)CC2)nc1 ; CN(C)CCOc1cnc(C(=O)N[C@H]2CC[C@@H](Oc3nc(N4CCOCC4)cc4ncccc34)CC2)nc1 ; CN(C)C(=N[C@H]1CC[C@@H](Oc2nc(N3CCOCC3)cc3ncccc23)CC1)N(C)C]

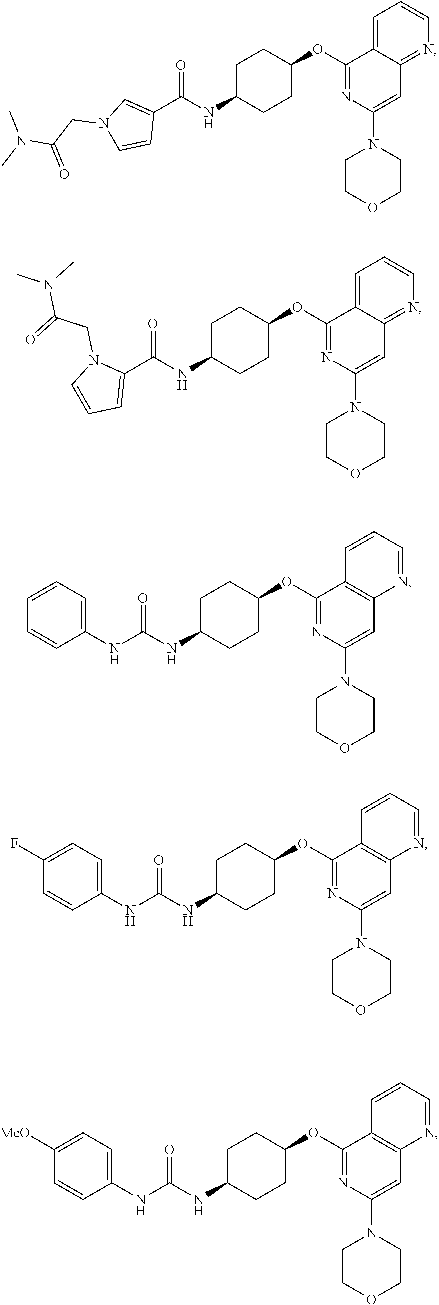 [SMILES: CN(C)C(=O)Cn1ccc(C(=O)N[C@H]2CC[C@@H](Oc3nc(N4CCOCC4)cc4ncccc34)CC2)c1 ; CN(C)C(=O)Cn1cccc1C(=O)N[C@H]1CC[C@@H](Oc2nc(N3CCOCC3)cc3ncccc23)CC1 ; O=C(Nc1ccccc1)N[C@H]1CC[C@@H](Oc2nc(N3CCOCC3)cc3ncccc23)CC1 ; O=C(Nc1ccc(F)cc1)N[C@H]1CC[C@@H](Oc2nc(N3CCOCC3)cc3ncccc23)CC1 ; COc1ccc(NC(=O)N[C@H]2CC[C@@H](Oc3nc(N4CCOCC4)cc4ncccc34)CC2)cc1]

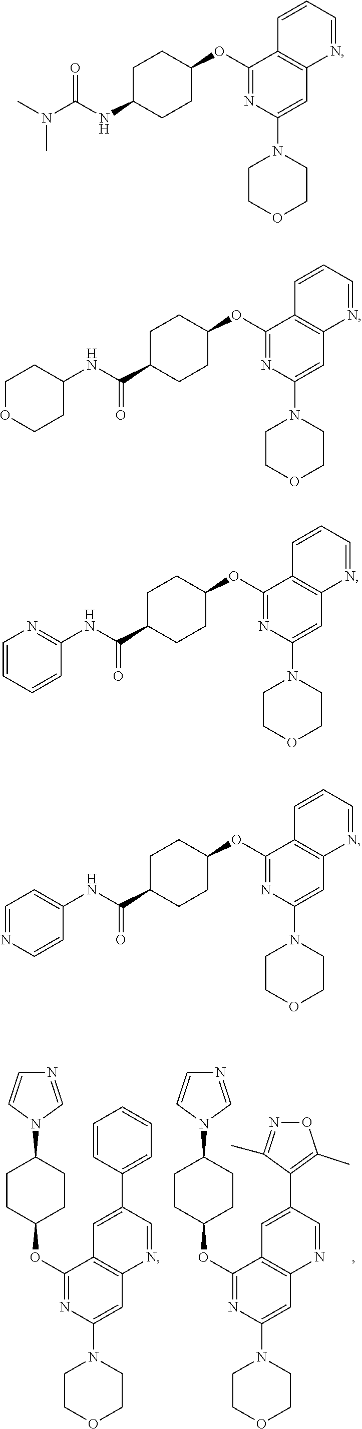

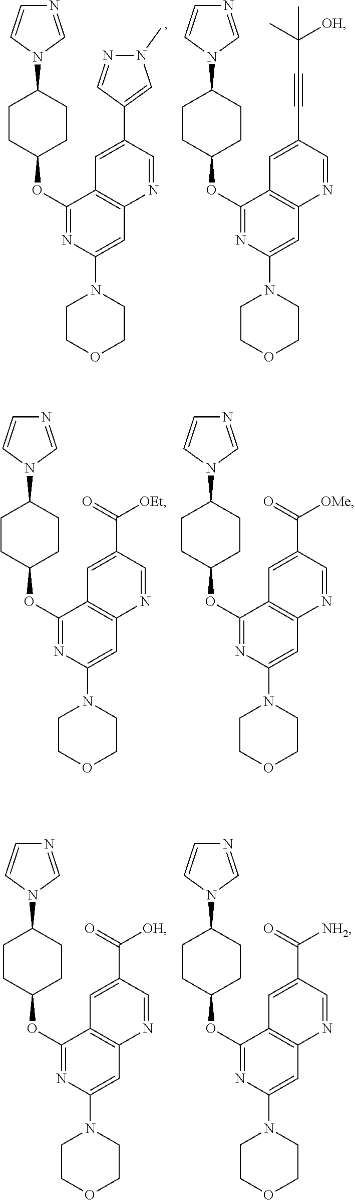

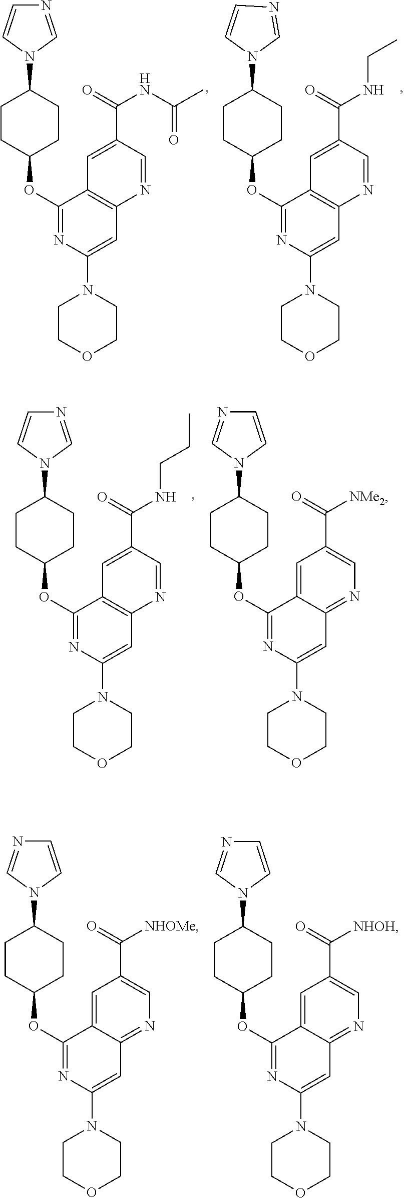

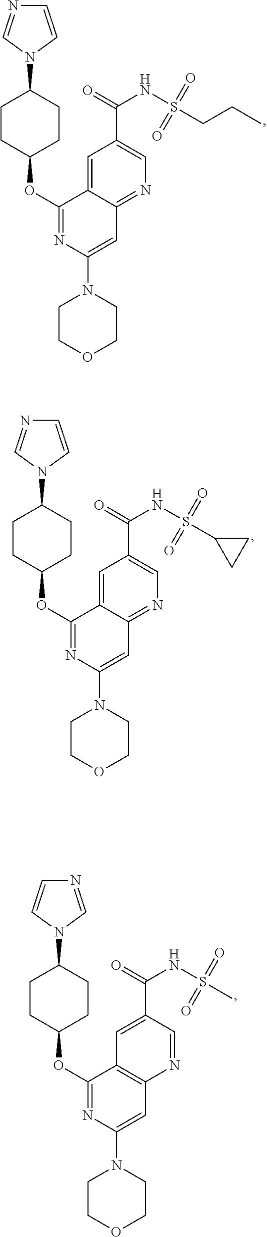 [SMILES: CCCS(=O)(=O)NC(=O)c1cnc2cc(N3CCOCC3)nc(O[C@H]3CC[C@@H](n4ccnc4)CC3)c2c1 ; O=C(NS(=O)(=O)C1CC1)c1cnc2cc(N3CCOCC3)nc(O[C@H]3CC[C@@H](n4ccnc4)CC3)c2c1 ; CS(=O)(=O)NC(=O)c1cnc2cc(N3CCOCC3)nc(O[C@H]3CC[C@@H](n4ccnc4)CC3)c2c1]

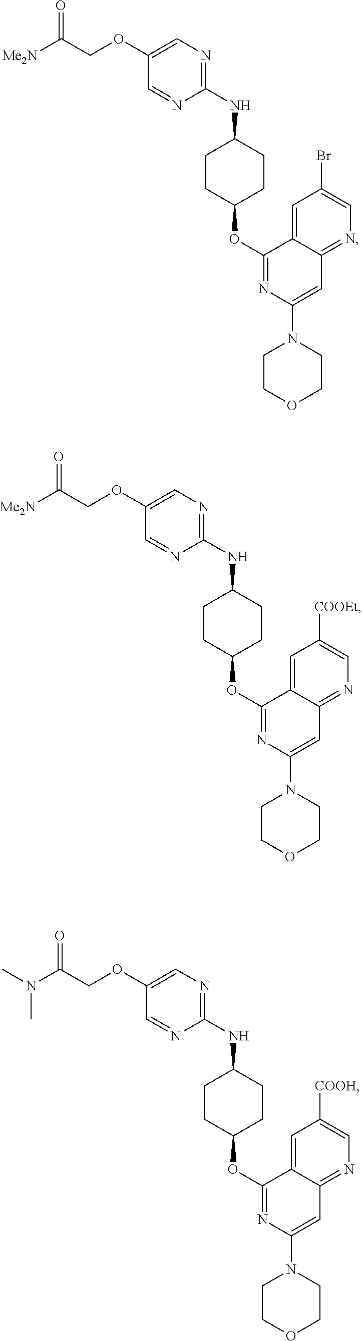 [SMILES: CN(C)C(=O)COc1cnc(N[C@H]2CC[C@@H](Oc3nc(N4CCOCC4)cc4ncc(Br)cc34)CC2)nc1 ; CCOC(=O)c1cnc2cc(N3CCOCC3)nc(O[C@H]3CC[C@@H](Nc4ncc(OCC(=O)N(C)C)cn4)CC3)c2c1 ; CN(C)C(=O)COc1cnc(N[C@H]2CC[C@@H](Oc3nc(N4CCOCC4)cc4ncc(C(=O)O)cc34)CC2)nc1]

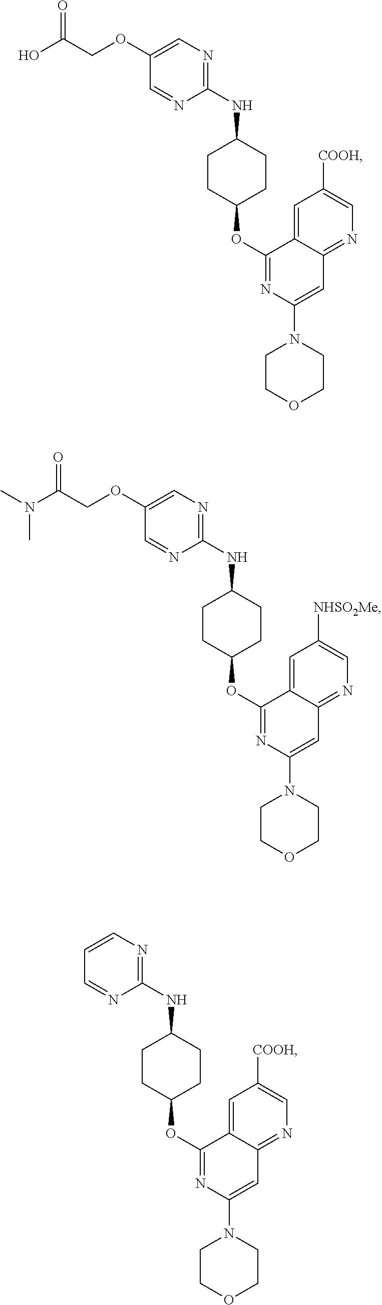

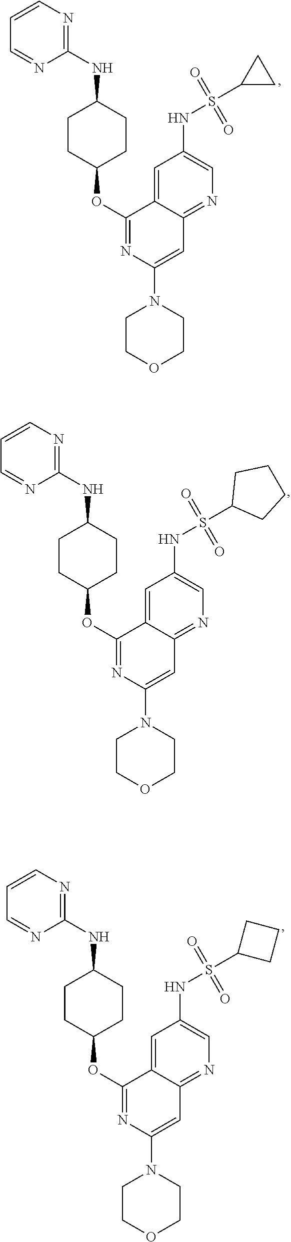 [SMILES: O=S(=O)(Nc1cnc2cc(N3CCOCC3)nc(O[C@H]3CC[C@@H](Nc4ncccn4)CC3)c2c1)C1CC1 ; O=S(=O)(Nc1cnc2cc(N3CCOCC3)nc(O[C@H]3CC[C@@H](Nc4ncccn4)CC3)c2c1)C1CCCC1 ; O=S(=O)(Nc1cnc2cc(N3CCOCC3)nc(O[C@H]3CC[C@@H](Nc4ncccn4)CC3)c2c1)C1CCC1]

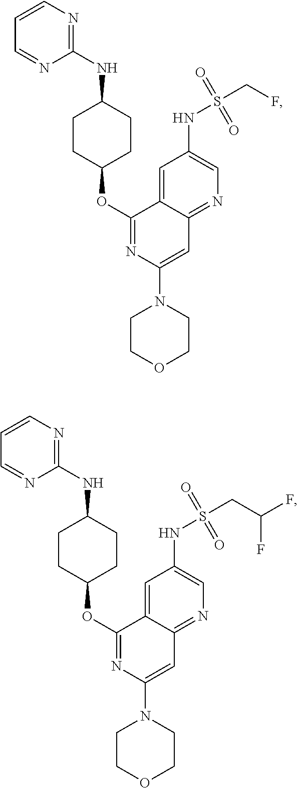 [SMILES: O=S(=O)(CF)Nc1cnc2cc(N3CCOCC3)nc(O[C@H]3CC[C@@H](Nc4ncccn4)CC3)c2c1 ; O=S(=O)(CC(F)F)Nc1cnc2cc(N3CCOCC3)nc(O[C@H]3CC[C@@H](Nc4ncccn4)CC3)c2c1]

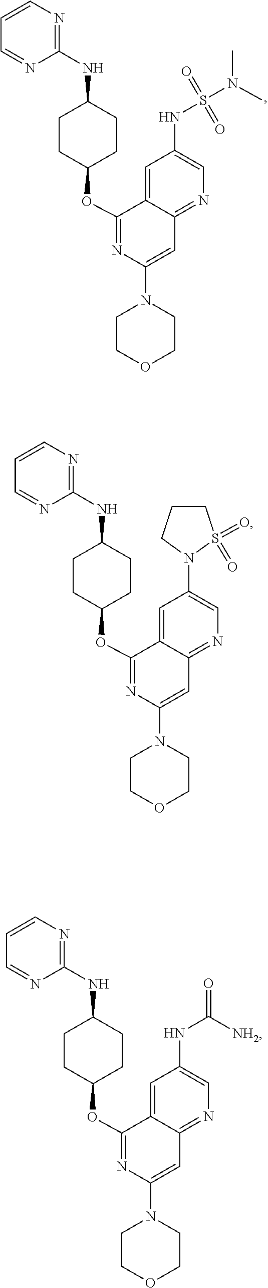 [SMILES: CN(C)S(=O)(=O)Nc1cnc2cc(N3CCOCC3)nc(O[C@H]3CC[C@@H](Nc4ncccn4)CC3)c2c1 ; O=S1(=O)CCCN1c1cnc2cc(N3CCOCC3)nc(O[C@H]3CC[C@@H](Nc4ncccn4)CC3)c2c1 ; NC(=O)Nc1cnc2cc(N3CCOCC3)nc(O[C@H]3CC[C@@H](Nc4ncccn4)CC3)c2c1]

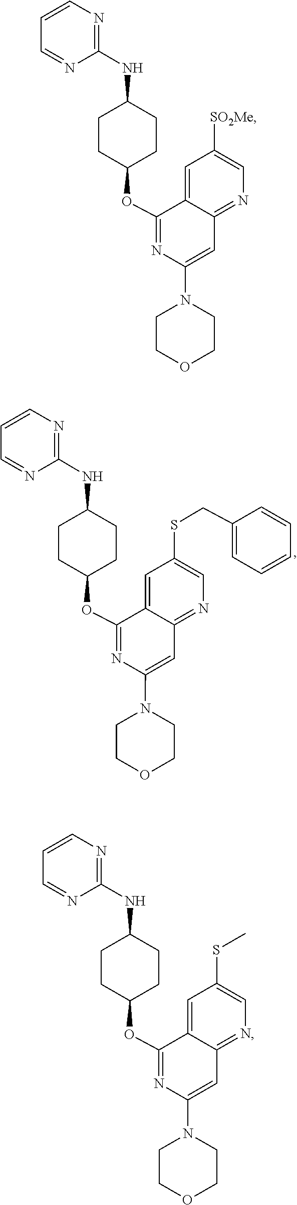 [SMILES: CS(=O)(=O)c1cnc2cc(N3CCOCC3)nc(O[C@H]3CC[C@@H](Nc4ncccn4)CC3)c2c1 ; c1ccc(CSc2cnc3cc(N4CCOCC4)nc(O[C@H]4CC[C@@H](Nc5ncccn5)CC4)c3c2)cc1 ; CSc1cnc2cc(N3CCOCC3)nc(O[C@H]3CC[C@@H](Nc4ncccn4)CC3)c2c1]

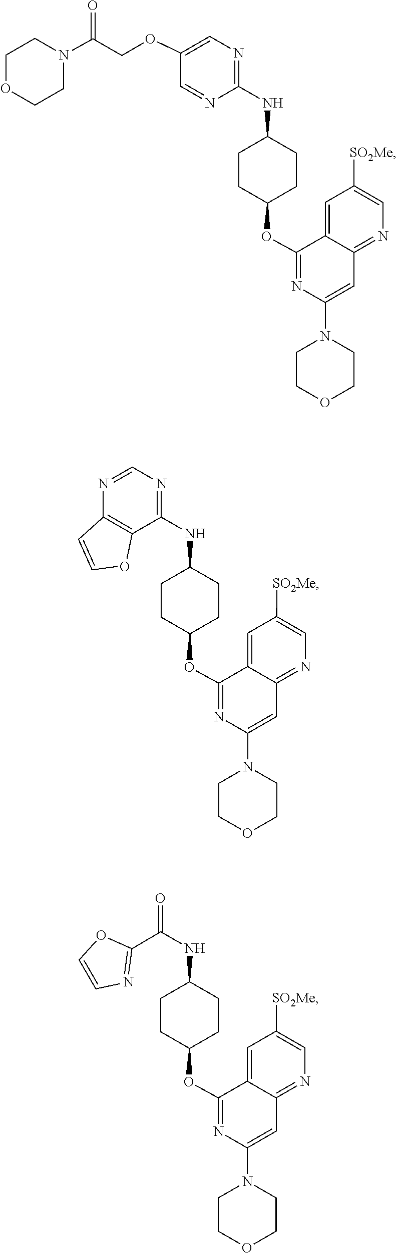 [SMILES: CS(=O)(=O)c1cnc2cc(N3CCOCC3)nc(O[C@H]3CC[C@@H](Nc4ncc(OCC(=O)N5CCOCC5)cn4)CC3)c2c1 ; CS(=O)(=O)c1cnc2cc(N3CCOCC3)nc(O[C@H]3CC[C@@H](Nc4ncnc5ccoc45)CC3)c2c1 ; CS(=O)(=O)c1cnc2cc(N3CCOCC3)nc(O[C@H]3CC[C@@H](NC(=O)c4ncco4)CC3)c2c1]

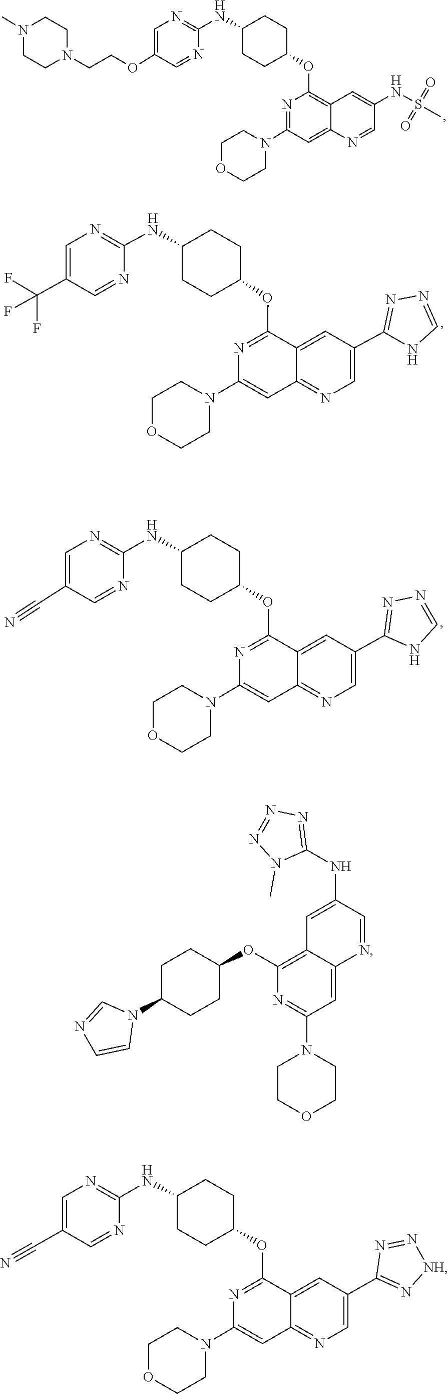

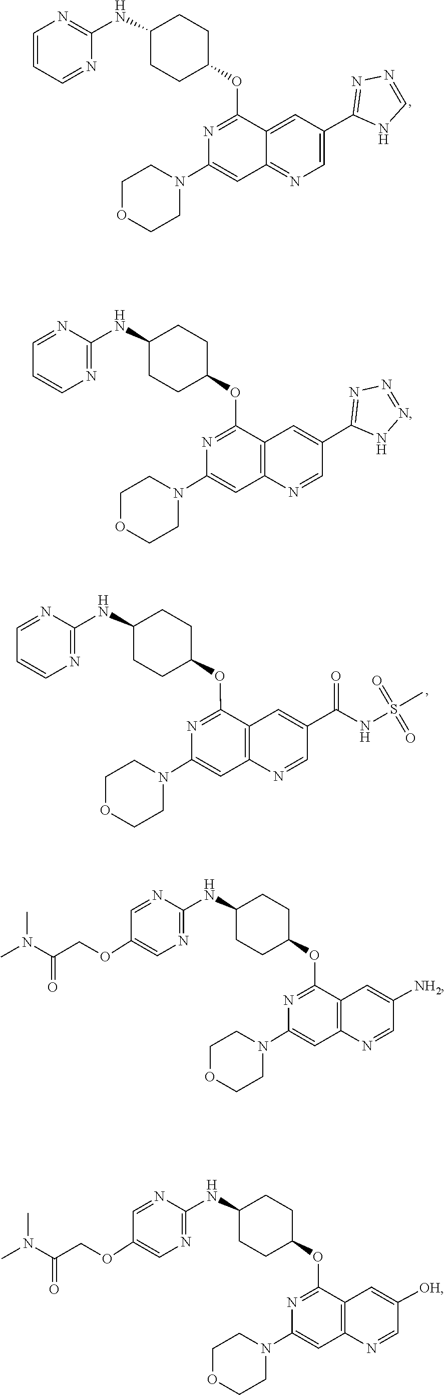

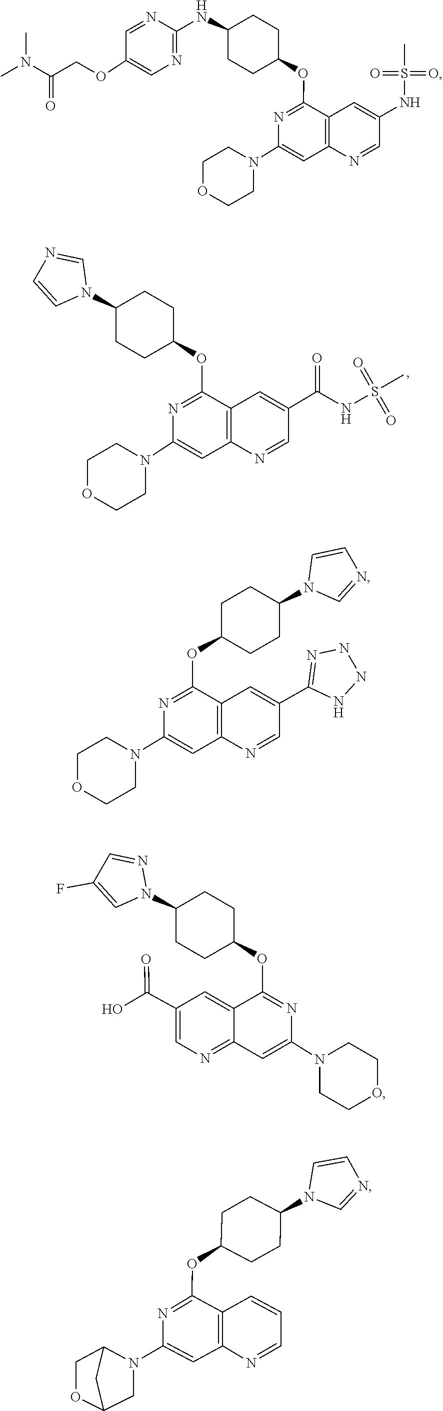

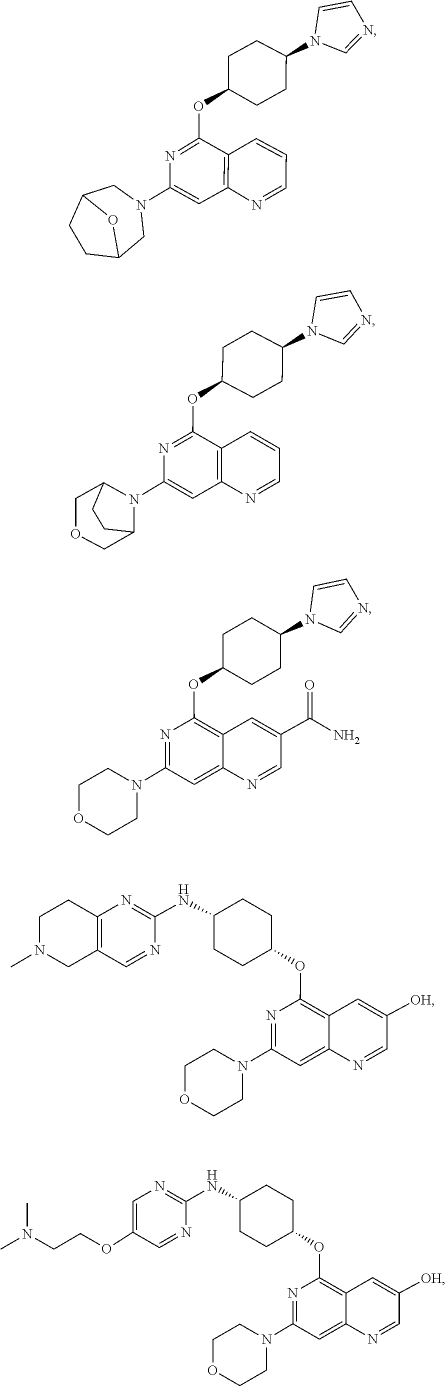 [SMILES: c1cnc2cc(N3CC4CCC(C3)O4)nc(O[C@H]3CC[C@@H](n4ccnc4)CC3)c2c1 ; c1cnc2cc(N3C4CCC3COC4)nc(O[C@H]3CC[C@@H](n4ccnc4)CC3)c2c1 ; NC(=O)c1cnc2cc(N3CCOCC3)nc(O[C@H]3CC[C@@H](n4ccnc4)CC3)c2c1 ; CN1CCc2nc(N[C@H]3CC[C@@H](Oc4nc(N5CCOCC5)cc5ncc(O)cc45)CC3)ncc2C1 ; CN(C)CCOc1cnc(N[C@H]2CC[C@@H](Oc3nc(N4CCOCC4)cc4ncc(O)cc34)CC2)nc1]

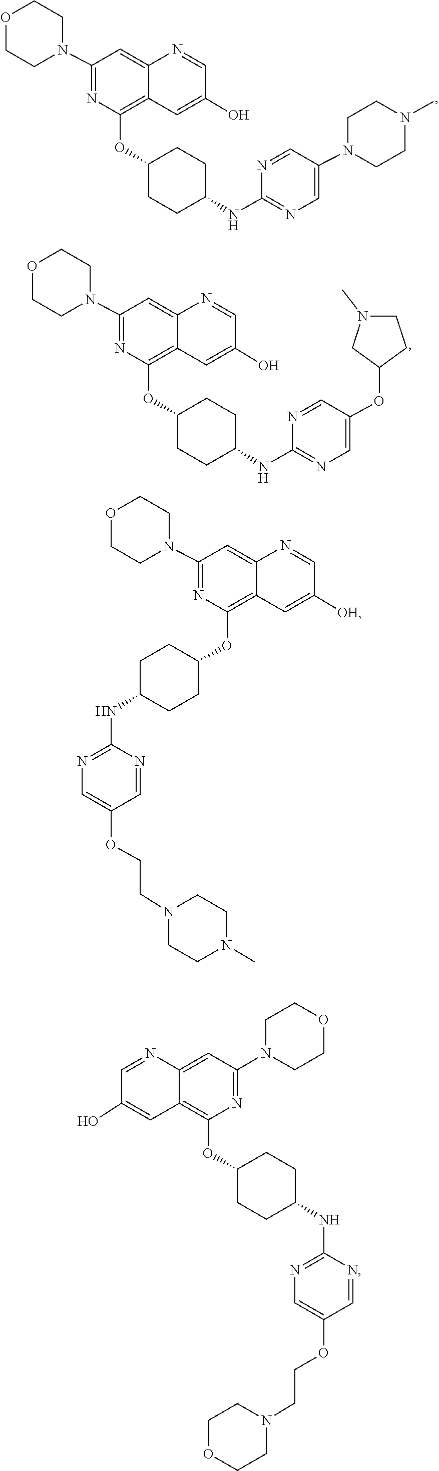 [SMILES: CN1CCN(c2cnc(N[C@H]3CC[C@@H](Oc4nc(N5CCOCC5)cc5ncc(O)cc45)CC3)nc2)CC1 ; CN1CCC(Oc2cnc(N[C@H]3CC[C@@H](Oc4nc(N5CCOCC5)cc5ncc(O)cc45)CC3)nc2)C1 ; CN1CCN(CCOc2cnc(N[C@H]3CC[C@@H](Oc4nc(N5CCOCC5)cc5ncc(O)cc45)CC3)nc2)CC1 ; Oc1cnc2cc(N3CCOCC3)nc(O[C@H]3CC[C@@H](Nc4ncc(OCCN5CCOCC5)cn4)CC3)c2c1]

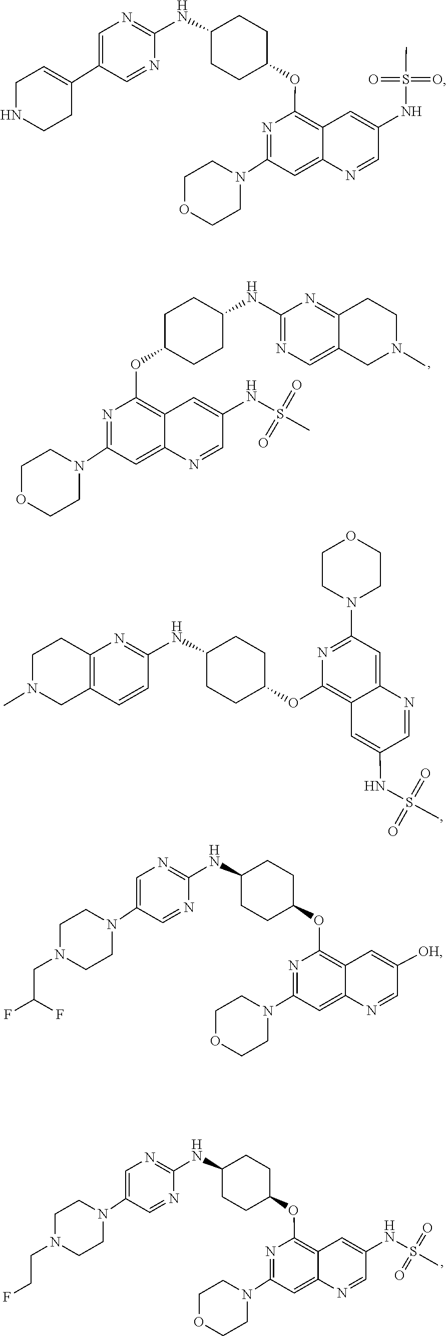

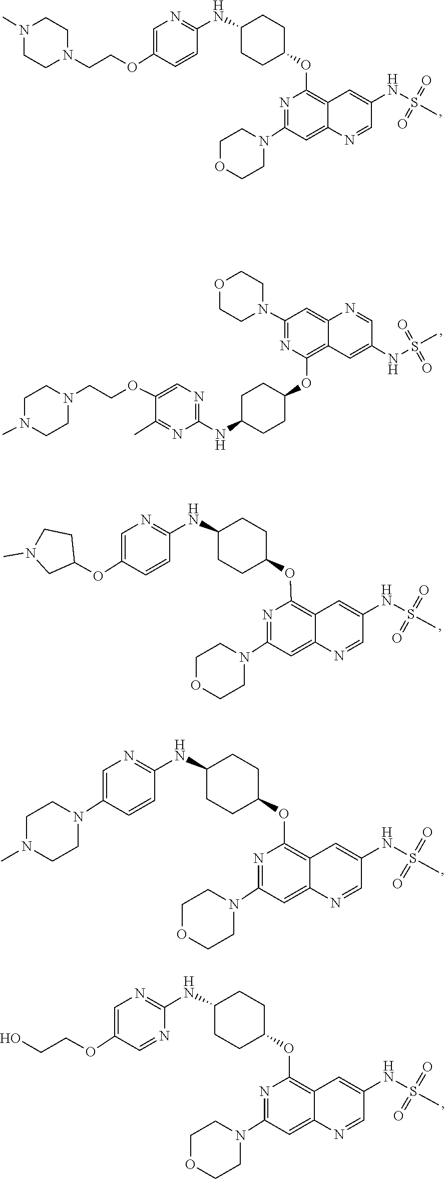

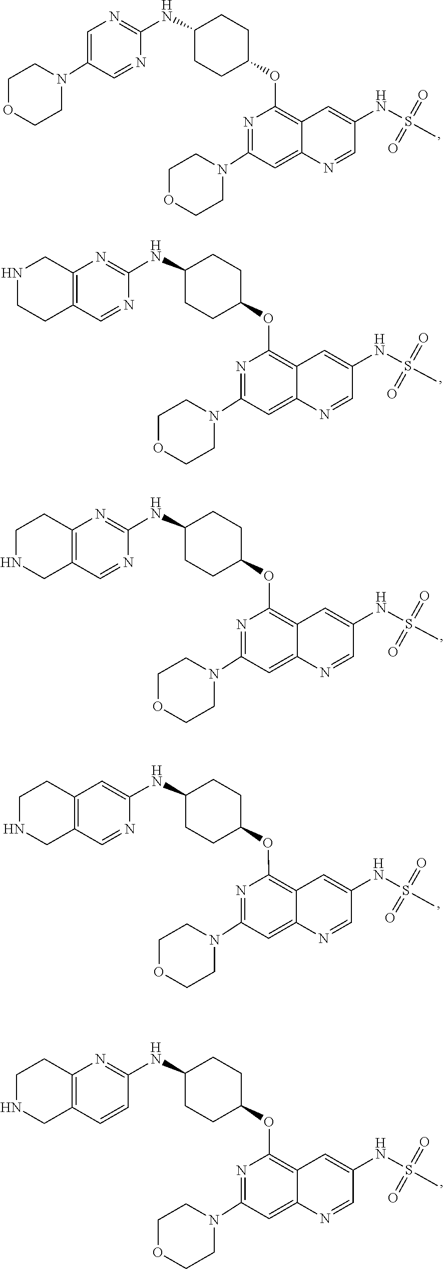

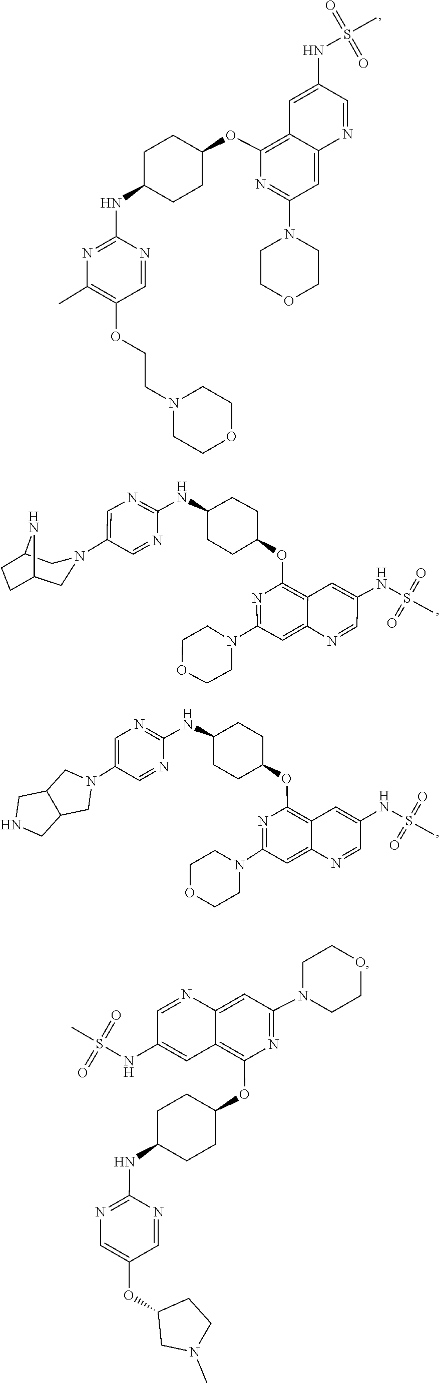

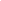

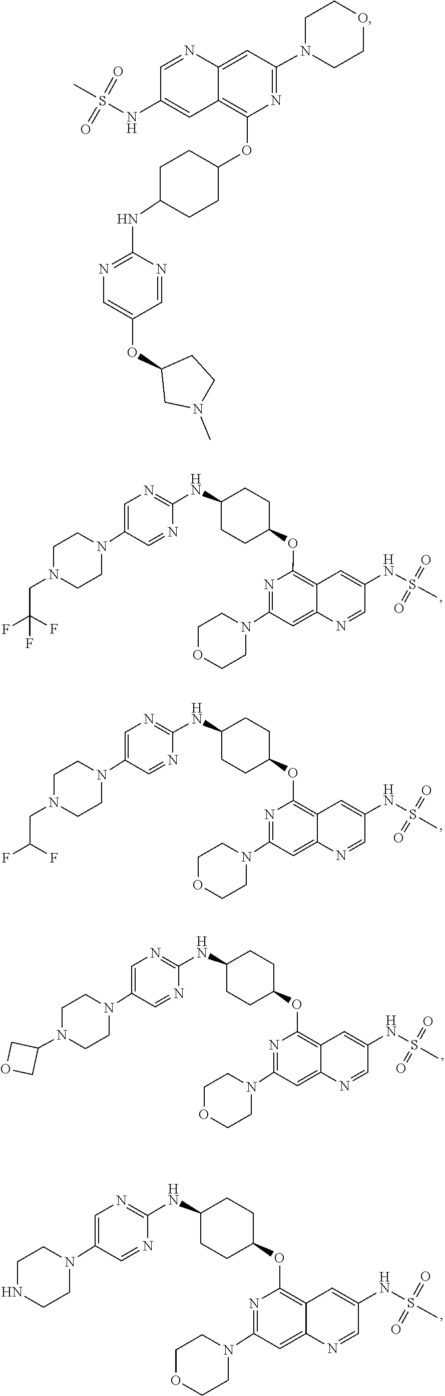 [SMILES: CN1CC[C@H](Oc2cnc(NC3CCC(Oc4nc(N5CCOCC5)cc5ncc(NS(C)(=O)=O)cc45)CC3)nc2)C1 ; CS(=O)(=O)Nc1cnc2cc(N3CCOCC3)nc(O[C@H]3CC[C@@H](Nc4ncc(N5CCN(CC(F)(F)F)CC5)cn4)CC3)c2c1 ; CS(=O)(=O)Nc1cnc2cc(N3CCOCC3)nc(O[C@H]3CC[C@@H](Nc4ncc(N5CCN(CC(F)F)CC5)cn4)CC3)c2c1 ; CS(=O)(=O)Nc1cnc2cc(N3CCOCC3)nc(O[C@H]3CC[C@@H](Nc4ncc(N5CCN(C6COC6)CC5)cn4)CC3)c2c1 ; CS(=O)(=O)Nc1cnc2cc(N3CCOCC3)nc(O[C@H]3CC[C@@H](Nc4ncc(N5CCNCC5)cn4)CC3)c2c1]

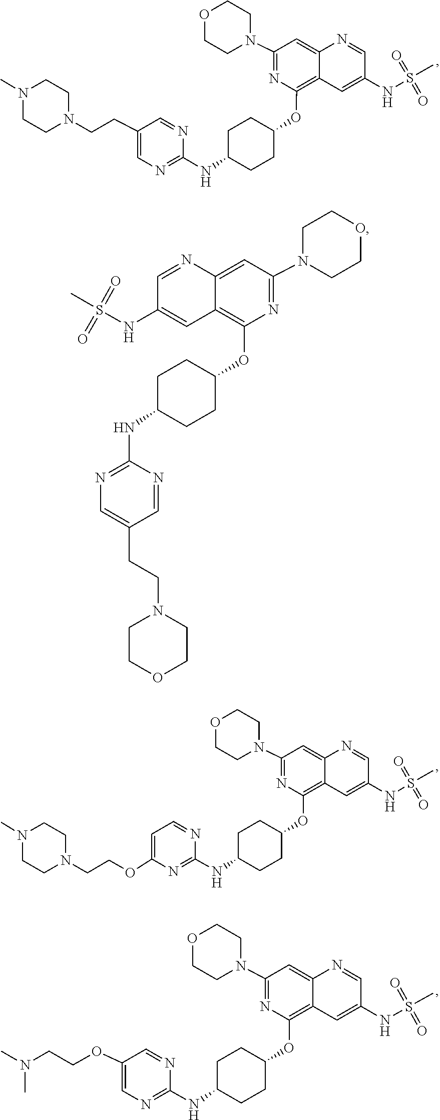

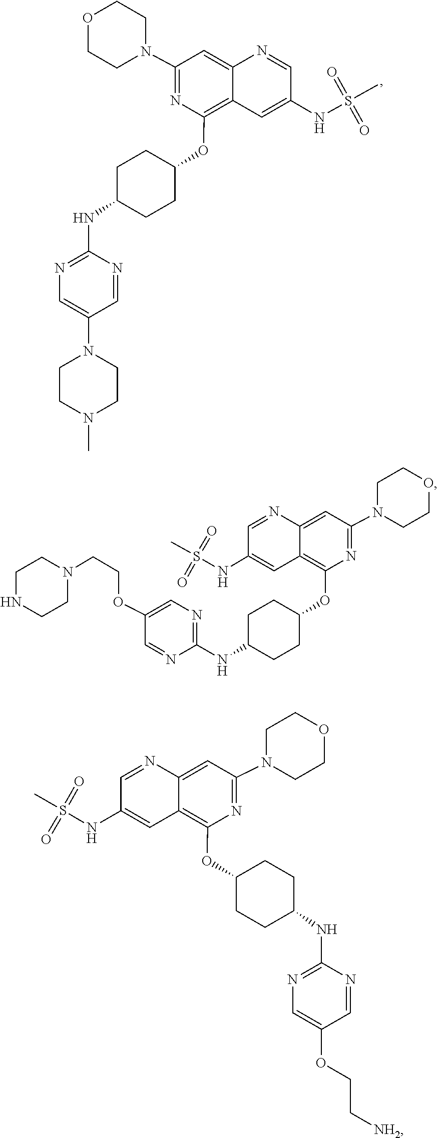

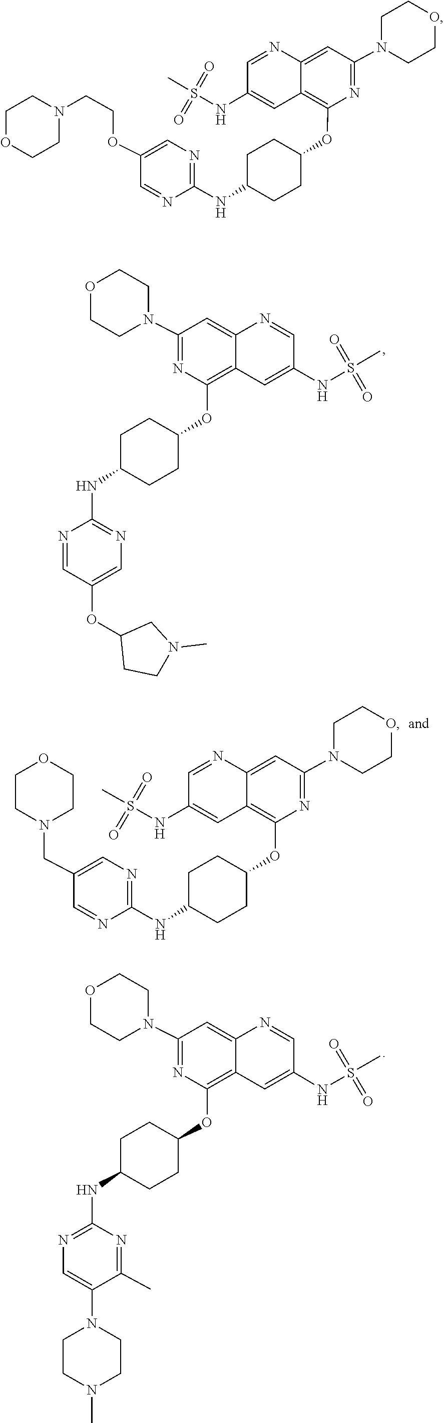

22. The compound of claim 1, wherein the compound is selected from:

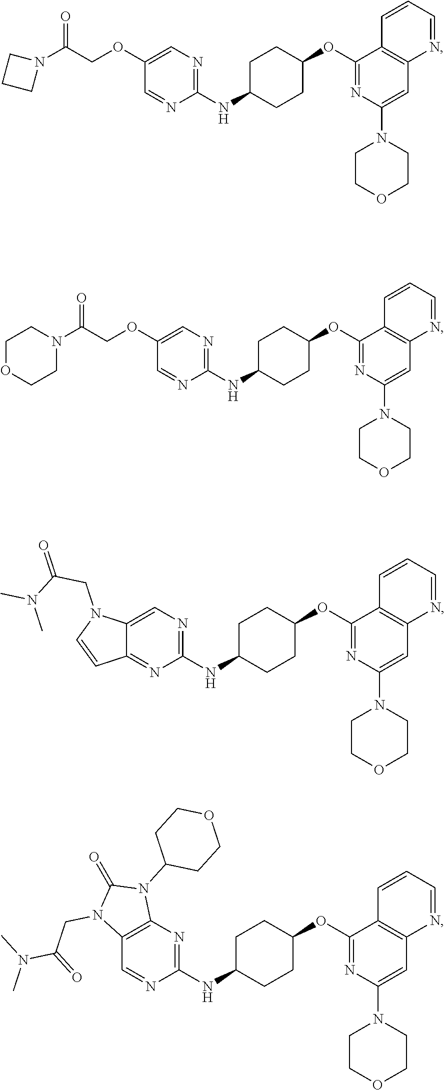 [SMILES: O=C(COc1cnc(N[C@H]2CC[C@@H](Oc3nc(N4CCOCC4)cc4ncccc34)CC2)nc1)N1CCC1 ; O=C(COc1cnc(N[C@H]2CC[C@@H](Oc3nc(N4CCOCC4)cc4ncccc34)CC2)nc1)N1CCOCC1 ; CN(C)C(=O)Cn1ccc2nc(N[C@H]3CC[C@@H](Oc4nc(N5CCOCC5)cc5ncccc45)CC3)ncc21 ; CN(C)C(=O)Cn1c(=O)n(C2CCOCC2)c2nc(N[C@H]3CC[C@@H](Oc4nc(N5CCOCC5)cc5ncccc45)CC3)ncc21]

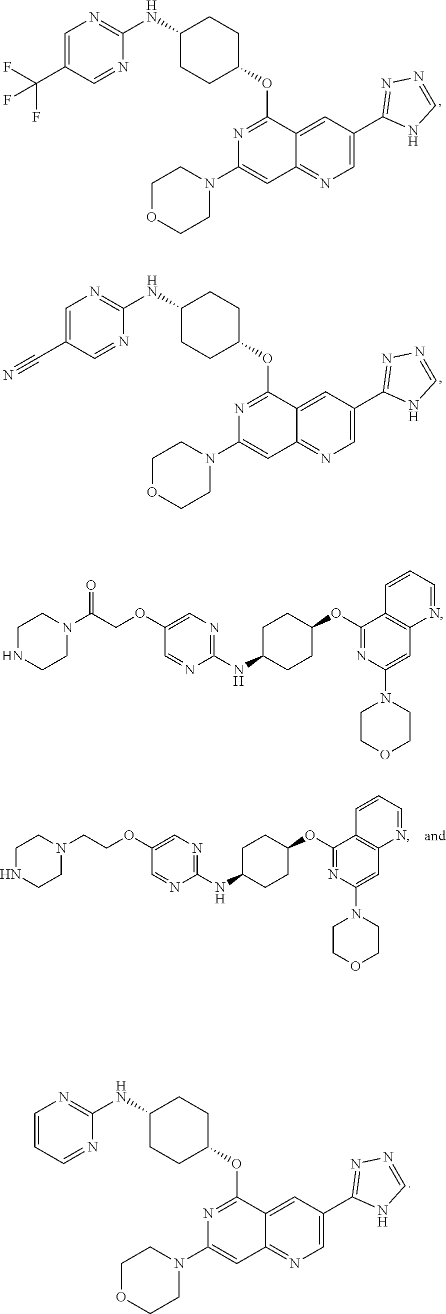

23. The compound of claim 1, wherein the compound is selected from:

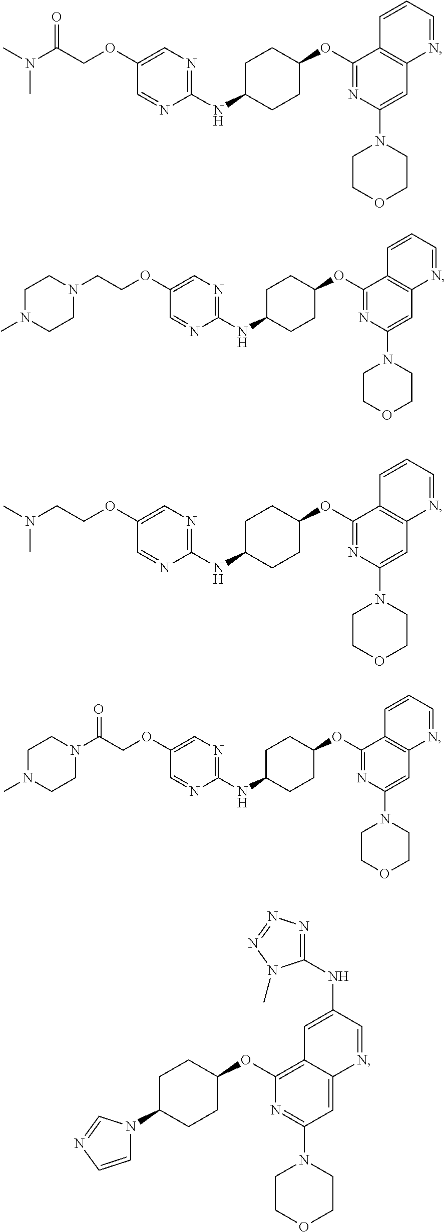 [SMILES: CN(C)C(=O)COc1cnc(N[C@H]2CC[C@@H](Oc3nc(N4CCOCC4)cc4ncccc34)CC2)nc1 ; CN1CCN(CCOc2cnc(N[C@H]3CC[C@@H](Oc4nc(N5CCOCC5)cc5ncccc45)CC3)nc2)CC1 ; CN(C)CCOc1cnc(N[C@H]2CC[C@@H](Oc3nc(N4CCOCC4)cc4ncccc34)CC2)nc1 ; CN1CCN(C(=O)COc2cnc(N[C@H]3CC[C@@H](Oc4nc(N5CCOCC5)cc5ncccc45)CC3)nc2)CC1 ; Cn1nnnc1Nc1cnc2cc(N3CCOCC3)nc(O[C@H]3CC[C@@H](n4ccnc4)CC3)c2c1]

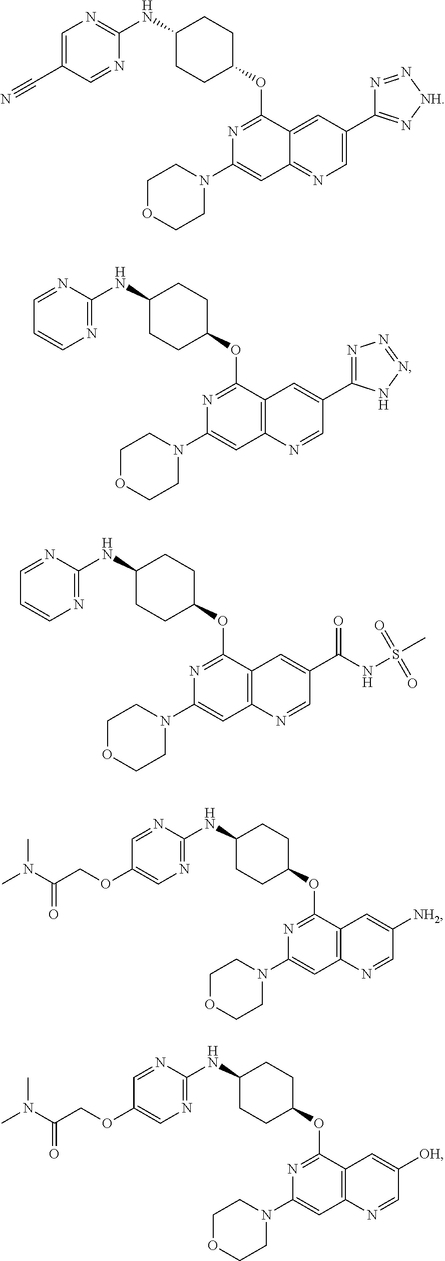

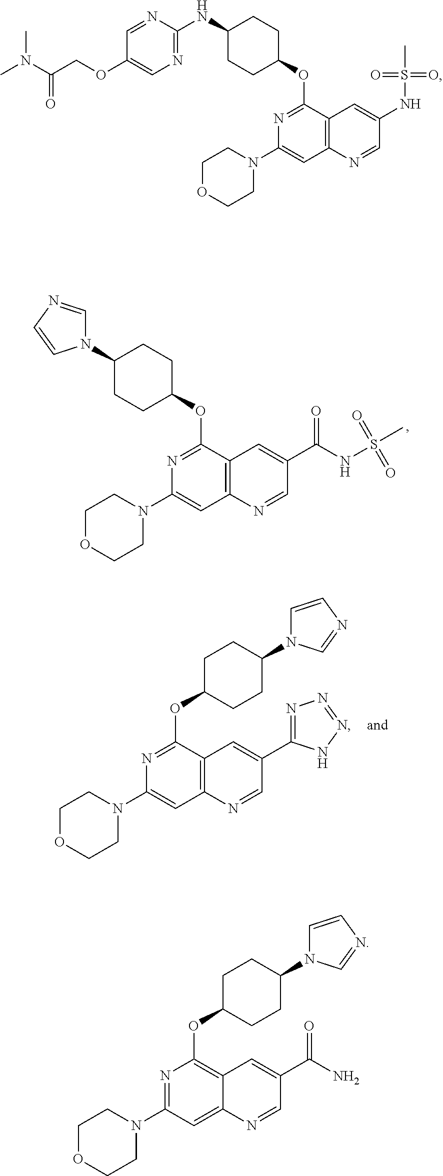 [SMILES: CN(C)C(=O)COc1cnc(N[C@H]2CC[C@@H](Oc3nc(N4CCOCC4)cc4ncc(NS(C)(=O)=O)cc34)CC2)nc1 ; CS(=O)(=O)NC(=O)c1cnc2cc(N3CCOCC3)nc(O[C@H]3CC[C@@H](n4ccnc4)CC3)c2c1 ; c1cn([C@H]2CC[C@@H](Oc3nc(N4CCOCC4)cc4ncc(-c5nnn[nH]5)cc34)CC2)cn1 ; NC(=O)c1cnc2cc(N3CCOCC3)nc(O[C@H]3CC[C@@H](n4ccnc4)CC3)c2c1]

24. The compound of claim 1, wherein the compound is a prodrug of a compound of formula (II) or a pharmaceutically acceptable salt thereof.

25.-28. (canceled)

29. A pharmaceutical composition comprising:
a compound of claim 1; and
a pharmaceutically-acceptable excipient.

30. A method of inhibiting DNA-PK activity comprising:
contacting DNA-PK with an effective amount of a compound of claim 1.

31. A method comprising:
administering to a subject an effective amount of a compound of claim 1.

32. A method of treating cancer comprising:
administering to a subject a therapeutically effective amount of a compound of claim 1.

33. The method of claim 32, wherein the method further comprises treating the subject with radiotherapy and/or a DNA damaging chemotherapeutic agent.

34. A method of repairing a DNA break in one or more target genomic regions via a homology directed repair (HDR) pathway, the method comprising:
administering to one or more cells that comprise one or more target genomic regions, a genome editing system, and a compound of claim 1,
wherein the genome editing system interacts with a nucleic acid of the one or more target genomic regions, resulting in a DNA break, and wherein the DNA break is repaired at least in part via a HDR pathway.

35. The method of claim 34, wherein the efficacy of the repair of the DNA break at the one or more target genomic regions via a HDR pathway is increased as compared to a cell in the absence of the compound.

36. A method of modifying expression of one or more genes or proteins, the method comprising:
administering to one or more cells that comprise one or more target genomic regions, a genome editing system, and a compound of claim 1,
wherein the genome editing system interacts with a nucleic acid of the one or more target genomic regions of a target gene, resulting in editing the one or more target genomic regions, and wherein the edit modifies expression of a downstream gene and/or protein associated with the target gene.

37. The method of claim 36, wherein the efficacy editing the one or more target genomic regions is increased as compared to a cell in the absence of the compound.

38. The method of claim 34, wherein the genome editing system is selected from a meganuclease based system, a zinc finger nuclease (ZFN) based system, a Transcription Activator-Like Effector-based Nuclease (TALEN) system, a CRISPR-based system, and a NgAgo-based system.

39. The method of claim 38, wherein the genome editing system is a CRISPR-based system.

40. The method of claim 39, wherein the CRISPR-based system is a CRISPR-Cas system or a CRISPR-Cpf system.
